(19) United States
(12) Patent Application Publication
(10) Pub. No.: US 2025/0042916 A1
(43) Pub. Date: Feb. 6, 2025

(54) COMPOUNDS, COMPOSITIONS, AND METHODS

(71) Applicant: ACONCAGUA BIO, INC., South San Francisco, CA (US)

(72) Inventors: Hui LEI, Shanghai (CN); Cui LI, Shanghai (CN); Chunliang LU, Shanghai (CN); Ding XUE, Shanghai (CN); Haizhen ZHANG, Shanghai (CN); Zhuming ZHANG, Shanghai (CN); Jian LIU, New York, NY (US)

(21) Appl. No.: 18/795,041

(22) Filed: Aug. 5, 2024

Related U.S. Application Data
(63) Continuation of application No. PCT/US2024/037786, filed on Jul. 12, 2024.

(30) Foreign Application Priority Data
Jul. 13, 2023 (WO) PCT/CN2023/107247
Feb. 8, 2024 (WO) PCT/CN2024/076848
Jun. 6, 2024 (WO) PCT/CN2024/097845

(51) Int. Cl. C07D 495/04; A61K 31/4439; A61K 31/444; A61K 31/4545; A61K 31/519; A61K 45/06; C07D 409/14; C07D 413/04; C07D 413/14; C07D 417/14; C07D 491/048

(52) U.S. Cl. CPC C07D 495/04; A61K 31/4439; A61K 31/444; A61K 31/4545; A61K 31/519; A61K 45/06; C07D 409/14; C07D 413/04; C07D 413/14; C07D 417/14; C07D 491/048

(57) ABSTRACT

The present disclosure provides compounds for modulating calcitonin receptor and/or amylin receptor activity, as well as pharmaceutical compositions comprising the compounds disclosed herein. Also provided are methods for treating a calcitonin receptor and/or amylin receptor associated disease or disorder.

### CROSS REFERENCE TO RELATED APPLICATIONS

This application claims priority to International Patent Application PCT/CN2023/107247 filed Jul. 13, 2023; International Patent Application PCT/CN2024/076848, filed Feb. 8, 2024; and International Patent Application PCT/CN2024/097845, filed Jun. 6, 2024, each of which is incorporated by reference in their entirety.

### FIELD

The present disclosure provides compounds for modulating calcitonin receptor and/or amylin receptor activity, as well as pharmaceutical compositions comprising the compounds disclosed herein. Also provided are methods for treating calcitonin receptor and/or amylin receptor associated diseases, disorders, and conditions.

### BACKGROUND

Calcitonin and amylin are hormones that interact with receptors within the same family to exert their effects on the human organism. Calcitonin, derived from thyroid C cells, is known for its inhibitory effect on osteoclasts. Calcitonin of mammalian origin promotes insulin sensitivity, while the more potent calcitonin extracted from salmon additionally inhibits gastric emptying, promotes gallbladder relaxation, increases energy expenditure and induces satiety as well as weight loss. Studies have also indicated that oral salmon calcitonin (sCT) exerts an insulin-sensitizing effect to improve glucose metabolism in obesity and type 2 diabetes. European Journal of Pharmacology, 2024, 737(7): 91-96.

Amylin receptors (AMYRs) are G protein-coupled receptors (GPCRs), which respond to the peptide hormones amylin and calcitonin. Amylin receptors are heterodimers comprising the calcitonin receptor, which is a G protein-coupled receptor, and one of three receptor-modifying proteins. Amylin, formed primarily in pancreatic islet β cells, is cosecreted with insulin in response to caloric intake. Patients with type 1 diabetes have lower baseline amylin serum concentrations, and amylin response to caloric intake is absent. Patients with type 2 diabetes requiring insulin also have a diminished amylin response to caloric intake, potentially related to the degree of β-cell impairment. Key physiologic functions of amylin in maintaining glucose homeostasis include suppressing glucagon release in response to caloric intake, delaying the rate of gastric emptying, and stimulating the satiety center in the brain to limit caloric intake.

The synthetic amylin analogue pramlintide is an approved treatment for diabetes mellitus as an adjunctive therapy to mealtime insulin which promotes better glycemic control and small but significant weight loss. AM833 (cagrilintide), an investigational novel long-acting acylated amylin analogue, acts as a non-selective amylin receptor agonist. This amylin receptor agonist can serve as an attractive novel treatment for obesity, resulting in reduction of food intake and significant weight loss in a dose-dependent manner. J Obes Metab Syndr. 2021; 30(4): 320-325.

Accordingly, modulators of the amylin and/or calcitonin receptor could be useful in treating various metabolic disorders, as well as inducing weight loss.

### SUMMARY

The present disclosure provides small molecule calcitonin and/or amylin receptor modulators (e.g., amylin-receptor agonists), as well as pharmaceutical compositions comprising the compounds disclosed herein. Also provided are methods for treating calcitonin receptor and/or amylin receptor associated diseases or disorders. It has been shown that calcitonin receptor activation is important for blood glucose regulation in diabetes; this is in addition to the known metabolic beneficial role of amylin receptor activation. Journal of Pharmacology and Experimental Therapeutics, 2020, 374 (1) 74-83.

This disclosure also provides pharmaceutical compositions comprising one or more compounds as disclosed herein, or a stereoisomer or mixture of stereoisomers thereof, or pharmaceutically acceptable salt thereof, and a pharmaceutically acceptable excipient.

Also provided herein are pharmaceutical compositions comprising one or more compounds as disclosed herein, or a stereoisomer or mixture of stereoisomers thereof, or pharmaceutically acceptable salt thereof, and a pharmaceutically acceptable excipient.

Also provided herein are methods for treating or preventing a calcitonin receptor and/or an amylin receptor associated disease or disorder in a subject in need thereof, the method comprising administering to a subject in need thereof a therapeutically effective amount of a compound disclosed herein (e.g., a compound of Formula I or subformula thereof, or a pharmaceutically acceptable salt, stereoisomer, mixture of stereoisomers, or solvate thereof), or a pharmaceutical composition thereof. In some embodiments, the method further comprises administering to the subject, a therapeutically effective amount of one or more additional therapy or therapeutic agent to the patient, such as, but not limited to, an antidiabetic agent, an anti-obesity agent, a weight loss agent, a GLP-1 receptor agonist, an anti-emetic agent, an agent to treat non-alcoholic steatohepatitis (NASH), gastric electrical stimulation, dietary monitoring, physical activity, or a combination thereof.

In some embodiments, the calcitonin receptor and/or amylin receptor associated disease or disorder is a bone disorder, a metabolic disorder, pain, a neurodegenerative disease or disorder, a cardiovascular disease, or other disease or disorder.

In some embodiments, the calcitonin receptor and/or amylin receptor associated disease or disorder is a bone disorder, including, but not limited to, osteoporosis, Paget's disease, hypercalcemia, Sudeck's atrophy, polystatic fibrous dysplasia, intersemocostoclavicular ossification, osteogenesis imperfecta, osteopenia, periodontal disease or defect, osteolytic bone disease, metastatic bone disorder, or bone loss resulting from a malignancy, autoimmune arthritides, a breakage or fracture, or immobility or disuse.

In some embodiments, the calcitonin receptor and/or amylin receptor associated disease or disorder is pain, including, but not limited to, osteopathic pain, phantom limb pain, general pain, hyperalgesia, or pain associated with diabetic neuropathy.

In some embodiments, the calcitonin receptor and/or amylin receptor associated disease or disorder is a neurodegenerative disease or disorder, including, but not limited to, Alzheimer's disease.

In some embodiments, the calcitonin receptor and/or amylin receptor associated disease or disorder is a metabolic disorder, including, but not limited to, non-alcoholic fatty liver disease (NAFLD), non-alcoholic steatohepatitis (NASH), insulin dependent diabetes, non-insulin dependent diabetes, impaired glucose tolerance, obesity, syndrome X, or other diabetic complication.

In some embodiments, the calcitonin receptor and/or amylin receptor associated disease or disorder is include primary or secondary hyperthyroidism, endocrine disorder, conditions associated with inhibiting gastric secretion, gastrointestinal disorders, renal osteodystrophy, or male infertility.

### BRIEF DESCRIPTION OF THE FIGURES

FIG. 1 illustrates the thermodynamic pathway used for calculating relative binding free energy. The relative binding free energy is calculated using two distinct transformations. First, the free energy of transforming ligand 1 to ligand 2 is determined in solvent; second, the free energy of transforming ligand 1 to ligand 2 is determined when bound to the target. The difference between these two values can be related to the binding free energy difference of the ligands 1 and 2.

FIG. 2 illustrates the thermodynamic factors involved in ligand binding to target. The left side indicates contributions due to ligand conformation and desolvation; the right side indicates the contributions due to target conformation and desolvation.

FIG. 3 illustrates the association between the in vitro Amylin Receptor cAMP Assay I and the in silico Computational Assay for representative compounds described herein and other compounds with known activity in the respective assays as described.

FIG. 4 illustrates the association between the in vitro Calcitonin receptor (CTR) cAMP Assay and the in silico Computational Assay for representative compounds described herein and other compounds with known activity in the respective assays as described.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line or a dashed line drawn through a line in a structure indicates a specified point of attachment of a group. Unless chemically or structurally required, no directionality or stereochemistry is indicated or implied by the order in which a chemical group is written or named.

The prefix “Cᵤ₋ᵥ” indicates that the following group has from u to v carbon atoms. For example, “C₁₋₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount±10%. In other embodiments, the term “about” includes the indicated amount±5%. In certain other embodiments, the term “about” includes the indicated amount±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁₋₂₀ alkyl), 1 to 12 carbon atoms (i.e., C₁₋₁₂ alkyl), 1 to 8 carbon atoms (i.e., C₁₋₈ alkyl), 1 to 6 carbon atoms (i.e., C₁₋₆ alkyl), or 1 to 4 carbon atoms (i.e., C₁₋₄ alkyl). Examples of alkyl groups include, e.g., methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂), and tert-butyl (i.e., —C(CH₃)₃), and “propyl” includes n-propyl (i.e., —(CH₂)₂CH₃), and isopropyl (i.e., —CH(CH₃)₂).

“Alkenyl” refers to an alkyl group containing at least one (e.g., 1-3, or 1) carbon-carbon double bond and having from 2 to 20 carbon atoms (i.e., C₂₋₂₀ alkenyl), 2 to 12 carbon atoms (i.e., C₂₋₁₂ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include, e.g., ethenyl, propenyl, butadienyl (including 1,2-butadienyl, and 1,3-butadienyl).

“Alkynyl” refers to an alkyl group containing at least one (e.g., 1-3, or 1) carbon-carbon triple bond and having from 2 to 20 carbon atoms (i.e., C₂₋₂₀ alkynyl), 2 to 12 carbon atoms (i.e., C₂₋₁₂ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond.

Certain commonly used alternative chemical names may be used. For example, a divalent group such as a divalent “alkyl” group, a divalent “aryl” group, etc., may also be referred to as an “alkylene” group or an “alkylenyl” group, an “arylene” group or an “arylenyl” group, respectively.

“Alkoxy” refers to the group “alkyl-O—”. Examples of alkoxy groups include, e.g., methoxy, ethoxy, n-propoxy, iso-propoxy, n-butoxy, tert-butoxy, sec-butoxy, n-pentoxy, n-hexoxy, and 1,2-dimethylbutoxy.

“Alkoxyalkyl” refers to an alkyl group as defined above, wherein a hydrogen atom is replaced by an alkoxy group as defined herein.

“Haloalkyl” refers to an unbranched or branched alkyl group as defined above, wherein one or more (e.g., 1 to 6 or 1 to 3) hydrogen atoms are replaced by an independently selected halo group. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Haloalkoxy” refers to an alkoxy group as defined above, wherein one or more (e.g., 1 to 6, or 1 to 3) hydrogen atoms are replaced by an independently selected halo group.

“Haloalkoxyalkyl” refers to an alkyl group as defined above, wherein a hydrogen atom is replaced by a haloalkoxy group as defined herein.

“Hydroxyalkyl” refers to an alkyl group as defined above, wherein one or more (e.g., 1 to 6, or 1 to 3) hydrogen atoms are replaced by a hydroxy group.

“Cyanoalkyl” refers to an alkyl group as defined above, wherein one, or one or more (e.g., 1 to 6, or 1 to 3) hydrogen atoms are replaced by cyano.

“Alkylthio” refers to the group “alkyl-S—”.

“Acyl” refers to a group —C(O)R, wherein R is hydrogen, alkyl, cycloalkyl, heterocyclyl, aryl, heteroalkyl, or heteroaryl; each of which may be optionally substituted, as defined herein. Examples of acyl include formyl, acetyl, cyclohexylcarbonyl, cyclohexylmethyl-carbonyl, and benzoyl.

“Amido” refers to both a “C-amido” group which refers to the group —C(O)NR^{y}R^{z} and an “N-amido” group which refers to the group —NR^{y}C(O)R^{z}, wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein, or R^{y} and R^{z} are taken together to form a cycloalkyl or heterocyclyl; each of which may be optionally substituted, as defined herein.

“Amino” refers to the group —NR^{y}R^{z} wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein.

“Amidino” refers to —C(NR^{y})(NR^{z}₂), wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include, e.g., phenyl, naphthyl, fluorenyl, and anthryl. Aryl, however, does not encompass or overlap in any way with heteroaryl defined below. If one or more aryl groups are fused with a heteroaryl, the resulting ring system is heteroaryl regardless of point of attachment. If one or more aryl groups are fused with a heterocyclyl, the resulting ring system is heterocyclyl regardless of point of attachment. If one or more aryl groups are fused with a cycloalkyl, the resulting ring system is cycloalkyl regardless of point of attachment.

“Carbamoyl” refers to both an “O-carbamoyl” group which refers to the group —O—C(O)NR^{y}R^{z} and an “N-carbamoyl” group which refers to the group —NR^{y}C(O)OR^{z}, wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein.

“Carboxyl ester” or “ester” refer to both —OC(O)R^{x} and —C(O)OR^{x}, wherein R^{x} is alkyl, alkenyl, alkynyl, cycloalkyl, heterocyclyl, aryl, heteroalkyl, or heteroaryl; each of which may be optionally substituted, as defined herein.

“Cycloalkyl” refers to a saturated or partially unsaturated cyclic alkyl group having a single ring or multiple rings including fused, bridged, and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₃₋₂₀ cycloalkyl), 3 to 14 ring carbon atoms (i.e., C₃₋₁₂ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Monocyclic groups include, for example, cyclopropyl, cyclobutyl, cyclopentyl, cyclohexyl, cycloheptyl, and cyclooctyl. Polycyclic groups include, for example, bicyclo[2.2.1]heptanyl, bicyclo[2.2.2]octanyl, adamantyl, norbornyl, decalinyl, 7,7-dimethyl-bicyclo[2.2.1]heptanyl, and the like. Further, the term cycloalkyl is intended to encompass any non-aromatic ring which may be fused to an aryl ring, regardless of the attachment to the remainder of the molecule (e.g., 2,3-dihydro-1H-indenyl). Still further, cycloalkyl also includes “spirocycloalkyl” when there are two positions for substitution on the same carbon atom, for example spiro[2.5]octanyl, spiro[4.5]decanyl, or spiro[5.5]undecanyl.

“Cycloalkylalkyl” refers to an alkyl group as defined above, wherein a hydrogen atom is replaced by a cycloalkyl group as defined herein.

“Imino” refers to a group —C(NR^{y})R^{z}, wherein R^{y} and R^{z} are each independently hydrogen, alkyl, alkenyl, alkynyl, cycloalkyl, heterocyclyl, aryl, heteroalkyl, or heteroaryl; each of which may be optionally substituted, as defined herein.

“Imido” refers to a group —C(O)NR^{y}C(O)R^{z} or —N(C(O)R^{y})C(O)R^{z}, wherein R^{y} and R^{z} are each independently hydrogen, alkyl, alkenyl, alkynyl, cycloalkyl, heterocyclyl, aryl, heteroalkyl, or heteroaryl; each of which may be optionally substituted, as defined herein, or R^{y} and R^{z} are taken together to form a heterocyclyl which may be optionally substituted, as defined herein.

“Halogen” or “halo” refers to atoms occupying group VIIA of the periodic table, such as fluoro, chloro, bromo, or iodo.

“Heteroalkyl” refers to an alkyl group in which one or more of the carbon atoms (and any associated hydrogen atoms) are each independently replaced with the same or different heteroatomic group. The term “heteroalkyl” includes unbranched or branched saturated chain having carbon and heteroatoms. By way of example, 1, 2 or 3 carbon atoms may be independently replaced with the same or different heteroatomic group. Heteroatomic groups include, but are not limited to, —NR—, —O—, —S—, —S(O)—, —S(O)₂—, and the like, where R is H, alkyl, aryl, cycloalkyl, heteroalkyl, heteroaryl or heterocyclyl, each of which may be optionally substituted. Examples of heteroalkyl groups include —OCH₃, —CH₂OCH₃, —SCH₃, —CH₂SCH₃, —NRCH₃, and —CH₂NRCH₃, where R is hydrogen, alkyl, aryl, arylalkyl, heteroalkyl, or heteroaryl, each of which may be optionally substituted. As used herein, heteroalkyl include 1 to 10 carbon atoms, 1 to 8 carbon atoms, or 1 to 4 carbon atoms; and 1 to 3 heteroatoms, 1 to 2 heteroatoms, or 1 heteroatom.

“Heteroalkylene” refers to a divalent heteroalkyl group. “Heteroalkylene” groups must have at least one carbon and at least one heteroatomic group within the chain. The term “heteroalkylene” includes unbranched or branched saturated chain having carbon and heteroatoms. By way of example, 1, 2, or 3 carbon atoms may be independently replaced with the same or different heteroatomic group. Heteroatomic groups include, but are not limited to, —NR^{y}—, —O—, —S—, —S(O)—, —S(O)₂—, and the like, wherein R^{y} is hydrogen, alkyl, alkenyl, alkynyl, cycloalkyl, heterocyclyl, aryl, heteroalkyl or heteroaryl; each of which may be optionally substituted, as defined herein. Examples of heteroalkylene groups include, e.g., —CH₂OCH₂—, —CH(CH₃)OCH₂—, —CH₂CH₂OCH₂—, —OCH₂—, —CH(CH₃)O—, —CH₂CH₂O—, —CH₂CH₂OCH₂CH₂OCH₂—, —CH₂CH₂OCH₂CH₂O—, —CH₂SCH₂—, —CH(CH₃)SCH₂—, —CH₂CH₂SCH₂—, —CH₂CH₂SCH₂CH₂SCH₂—, —SCH₂—, —CH(CH₃)S—, —CH₂CH₂S—, —CH₂CH₂SCH₂CH₂S—, —CH₂S(O)₂CH₂—, —CH(CH₃)S(O)₂CH₂—, —CH₂CH₂S(O)₂CH₂—, —CH₂CH₂S(O)₂CH₂CH₂OCH₂—, —CH₂NR^{y}CH₂—, —CH(CH₃)NR^{y}CH₂—, —CH₂CH₂NR^{y}CH₂—, —CH₂CH₂NR^{y}CH₂CH₂NR^{y}CH₂—, etc., where R^{y} is hydrogen, alkyl, alkenyl, alkynyl, cycloalkyl, heterocyclyl, aryl, heteroalkyl, or heteroaryl; each of which may be optionally substituted, as defined herein). As used herein, heteroalkylene includes 1 to 10 carbon atoms, 1 to 8 carbon atoms, or 1 to 4 carbon atoms; and 1 to 3 heteroatoms, 1 to 2 heteroatoms, or 1 heteroatom. As used herein, the term “heteroalkylene” does not include groups such as amides or other functional groups having an oxo present on one or more carbon atoms.

“Heteroaryl” refers to an aromatic group having a single ring or multiple fused rings, with one or more ring heteroatoms independently selected from nitrogen, oxygen, and sulfur. As used herein, heteroaryl includes 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen, and sulfur. In certain instances, heteroaryl includes 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen, and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl, benzotriazolyl, benzo[4,6]imidazo[1,2-a]pyridyl, carbazolyl, cinnolinyl, dibenzofuranyl, dibenzothienyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, thienyl, triazolyl, tetrazolyl, and triazinyl. Examples of the fused-heteroaryl rings include, but are not limited to, benzo[d]thiazolyl, quinolinyl, isoquinolinyl, benzo[b]thienyl, indazolyl, benzo[d]imidazolyl, pyrazolo[1,5-a]pyridinyl, and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heterocyclyl” refers to a saturated or partially unsaturated cyclic alkyl group, with one or more ring heteroatoms independently selected from nitrogen, oxygen, and sulfur. The term “heterocyclyl” includes heterocycloalkenyl groups (i.e., the heterocyclyl group having at least one double bond), bridged-heterocyclyl groups, fused-heterocyclyl groups, and spiro-heterocyclyl groups. A heterocyclyl may be a single ring or multiple rings wherein the multiple rings may be fused, bridged, or spiro, and may comprise one or more (e.g., 1 to 3) oxo (═O) (e.g., —C(O)—, —S(O)—, —S(O)₂—, or —P(O)—) or N-oxide (—O⁻) moieties. Any non-aromatic ring or fused ring system containing at least one heteroatom and one non-aromatic ring is considered a heterocyclyl, regardless of the attachment to the remainder of the molecule. For example, fused ring systems such as 6,7-dihydro-5H-cyclopenta[b]pyridinyl, decahydroquinazolinyl, 1,2,3,4-tetrahydroquinazolinyl, and 5,6,7,8-tetrahydroquinazolinyl are heterocyclyl, regardless of the attachment (i.e., can be bound through a carbon atom or a heteroatom). Further, the term heterocyclyl is intended to encompass any non-aromatic ring containing at least one heteroatom, which ring may be fused to a cycloalkyl, an aryl, or heteroaryl ring, regardless of the attachment to the remainder of the molecule. As used herein, heterocyclyl has 2 to 20 ring carbon atoms (i.e., C₂₋₂₀ heterocyclyl), 2 to 12 ring carbon atoms (i.e., C₂₋₁₂ heterocyclyl), 2 to 10 ring carbon atoms (i.e., C₂₋₁₀ heterocyclyl), 2 to 8 ring carbon atoms (i.e., C₂₋₈ heterocyclyl), 3 to 12 ring carbon atoms (i.e., C₃₋₁₂ heterocyclyl), 3 to 8 ring carbon atoms (i.e., C₃₋₈ heterocyclyl), or 3 to 6 ring carbon atoms (i.e., C₃₋₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur, or oxygen. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiomorpholinyl, thiamorpholinyl, 1-oxo-thiomorpholinyl, and 1,1-dioxo-thiomorpholinyl. The term “heterocyclyl” also includes “spiroheterocyclyl” when there are two positions for substitution on the same carbon atom. Examples of the spiro-heterocyclyl rings include, e.g., bicyclic and tricyclic ring systems, such as oxabicyclo[2.2.2]octanyl, 2-oxa-7-azaspiro[3.5]nonanyl, 2-oxa-6-azaspiro[3.4]octanyl, and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl, and isoindolinyl, where the heterocyclyl can be bound via either ring of the fused system.

“Sulfonyl” refers to the group —S(O)₂R^{y}, where R^{y} is hydrogen, alkyl, alkenyl, alkynyl, cycloalkyl, heterocyclyl, aryl, heteroalkyl, or heteroaryl; each of which may be optionally substituted, as defined herein. Examples of sulfonyl are methylsulfonyl, ethylsulfonyl, phenylsulfonyl, and toluenesulfonyl.

“Sulfinyl” refers to the group —S(O)R^{y}, where R^{y} is hydrogen, alkyl, alkenyl, alkynyl, cycloalkyl, heterocyclyl, aryl, heteroalkyl, or heteroaryl; each of which may be optionally substituted, as defined herein.

The terms “optional” or “optionally” means that the subsequently described event or circumstance may or may not occur, and that the description includes instances where said event or circumstance occurs and instances in which it does not. Also, the term “optionally substituted” refers to any one or more (e.g., 1 to 5, or 1 to 3) hydrogen atoms on the designated atom or group may or may not be replaced by a moiety other than hydrogen.

As used herein, the term “compound,” is meant to include any or all stereoisomers, geometric isomers, tautomers, and isotopically enriched analogs (e.g., deuterated analogs) of the structures depicted. Compounds herein identified by name or structure as one particular tautomeric form are intended to include other tautomeric forms unless otherwise specified.

Some of the compounds exist as tautomers. Tautomers are in equilibrium with one another. For example, amide containing compounds may exist in equilibrium with imidic acid tautomers. Regardless of which tautomer is shown, and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

Any compound or structure given herein, is also intended to represent unlabeled forms as well as isotopically labeled forms of the compounds. These forms of compounds may also be referred to as “isotopically enriched analogs.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Various isotopically labeled compounds of the present disclosure, for example those into which radioactive isotopes such as ³H and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The term “isotopically enriched analogs” includes “deuterated analogs” of compounds described herein in which one or more hydrogens is/are replaced by deuterium, such as a hydrogen on a carbon atom. Such compounds exhibit increased resistance to metabolism and are thus useful for increasing the half-life of any compound when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism, and excretion (ADME). Substitution with heavier isotopes such as deuterium may afford certain therapeutic advantages resulting from greater metabolic stability, for example increased in vivo half-life, reduced dosage requirements, and/or an improvement in therapeutic index. An ¹⁸F, ³H, ¹¹C labeled compound may be useful for PET or SPECT or other imaging studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Accordingly, in the compounds of this disclosure any atom specifically designated as a deuterium (D) is meant to represent deuterium.

In many cases, the compounds of this disclosure are capable of forming acid and/or base salts by virtue of the presence of amino and/or carboxyl groups or groups similar thereto.

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include, e.g., hydrochloric acid, hydrobromic acid, sulfuric acid, nitric acid, phosphoric acid and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, aluminum, ammonium, calcium, and magnesium salts. Salts derived from organic bases include, but are not limited to, salts of NH₃, or primary, secondary, tertiary amines, such as salts derived from a N-containing heterocycle, a N-containing heteroaryl, or derived from an amine of formula N(R^{N})₃ (e.g., HN⁺(R^{N})₃ or (alkyl)N⁺(R^{N})₃) where each R^{N} is independently hydrogen, alkyl, alkenyl, alkynyl, haloalkyl, cycloalkyl, heterocyclyl, aryl, or heteroaryl, wherein each is optionally substituted, such as by one or more (e.g., 1-5 or 1-3) substituents (e.g., halo, cyano, hydroxy, amino, alkyl, alkenyl, alkynyl, haloalkyl, alkoxy, or haloalkoxy). Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

The term “substituted” means that any one or more hydrogen atoms on the designated atom or group is replaced with one or more substituents other than hydrogen, provided that the designated atom's normal valence is not exceeded. The one or more substituents include, but are not limited to, acyl, alkenyl, alkoxy, alkoxyalkyl, alkyl, alkylthio, alkynyl, amidino, amido, amino, aryl, azido, carbamoyl, carboxyl, carboxyl ester, cyano, cyanoalkyl, cycloalkyl, cycloalkylalkyl, guanidino, halo, haloalkoxy, haloalkoxyalkyl, haloalkyl, heteroalkyl, heteroaryl, heterocyclyl, hydrazino, hydroxy, hydroxyalkyl, imido, imino, nitro, oxo, sulfinyl, sulfonic acid, sulfonyl, thiocyanate, thiol, thione, or combinations thereof.

Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl) substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein. Unless specified otherwise, where a group is described as optionally substituted, any substituents of the group are themselves unsubstituted. For example, in some embodiments, the term “substituted alkyl” refers to an alkyl group having one or more substituents including hydroxy, halo, alkoxy, cycloalkyl, heterocyclyl, aryl, and heteroaryl. In other embodiments, the one or more substituents may be further substituted with halo, alkyl, haloalkyl, hydroxy, alkoxy, cycloalkyl, heterocyclyl, aryl, or heteroaryl, each of which is substituted. In other embodiments, the substituents may be further substituted with halo, alkyl, haloalkyl, alkoxy, hydroxy, cycloalkyl, heterocyclyl, aryl, or heteroaryl, each of which is unsubstituted.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

A “solvate” is formed by the interaction of a solvent and a compound. Solvates of salts of the compounds described herein are also provided. Hydrates of the compounds described herein are also provided.

The term “pharmaceutically acceptable” as used herein indicates that the compound, or salt or composition thereof is compatible chemically and/or toxicologically with the other ingredients comprising a formulation and/or the subject being treated therewith.

The term “administration” or “administering” refers to a method of giving a dosage of a compound or pharmaceutical composition to a vertebrate or invertebrate, including a mammal, a bird, a fish, or an amphibian. The method of administration can vary depending on various factors, e.g., the components of the pharmaceutical composition, the site of the disease, and the severity of the disease.

The terms “effective amount” or “effective dosage” or “pharmaceutically effective amount” or “therapeutically effective amount,” as used herein, refer to a sufficient amount of a chemical entity (e.g., a compound of Formula I, or a pharmaceutically acceptable salt, stereoisomer, mixture of stereoisomers, or solvate thereof) being administered which will relieve to some extent one or more of the symptoms of the disease or condition being treated, and can include curing the disease. “Curing” means that the symptoms of active disease are eliminated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study. In some embodiments, a “therapeutically effective amount” of a compound as provided herein refers to an amount of the compound that is effective as a monotherapy or combination therapy.

The term “excipient” or “pharmaceutically acceptable excipient” means a pharmaceutically-acceptable material, composition, or vehicle, such as a liquid or solid filler, diluent, carrier, solvent, or encapsulating material. In some embodiments, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutical composition” refers to a mixture of a compound of Formula I, or a pharmaceutically acceptable salt, stereoisomer, mixture of stereoisomers, or solvate thereof as provided herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to, rectal, oral, intravenous, aerosol, parenteral, ophthalmic, pulmonary, and topical administration.

The term “calcitonin receptor and/or amylin receptor associated disease or disorder” as used herein is meant to include, without limitation, those diseases, disorders, or conditions in which activation of at least one calcitonin receptor (CTR) and/or amylin receptor (AMY) by calcitonin and/or amylin contributes to the symptomology or progression of the disease or disorder. These diseases or disorders may arise from one or more of a genetic, iatrogenic, immunological, infectious, metabolic, oncological, toxic, surgical, and/or traumatic etiology.

The terms “treat,” “treating,” and “treatment,” in the context of treating a disease, disorder, or condition, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof.

The term “preventing”, as used herein, is the prevention of the onset, recurrence or spread, in whole or in part, of the disease or condition as described herein, or a symptom thereof.

The terms “subject,” “patient,” or “individual,” as used herein, are used interchangeably and refers to any animal, including mammals such as mice, rats, other rodents, rabbits, dogs, cats, swine, cattle, sheep, horses, primates, and humans. In some embodiments, the term refers to a subject, particularly a mammalian subject, for whom diagnosis, prognosis, or therapy is desired or needed. In some embodiments, the subject is a human. In some embodiments, the subject has experienced and/or exhibited at least one symptom of the disease, disorder, or condition to be treated and/or prevented.

The terms “treatment regimen” and “dosing regimen” are used interchangeably to refer to the dose and timing of administration of each therapeutic agent in a combination.

The term “pharmaceutical combination,” as used herein, refers to a pharmaceutical treatment resulting from the mixing or combining of more than one active ingredient and includes both fixed and non-fixed combinations of the active ingredients.

The term “combination therapy” as used herein refers to a dosing regimen of two different therapeutically active agents (i.e., the components or combination partners of the combination), wherein the therapeutically active agents are administered together or separately in a manner prescribed by a medical care taker or according to a regulatory agency as defined herein.

The term “modulate,” “modulating,” or “modulation,” as used herein, refers to a regulation or an adjustment (e.g., increase or decrease) and can include, for example agonism, partial agonism or antagonism.

### Compounds

Provided herein are compounds that are amylin modulators.

In some embodiments, provided is a compound of Formula I:

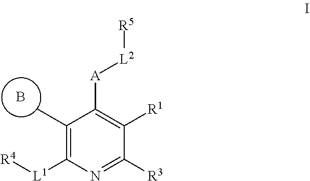

or a pharmaceutically acceptable salt, stereoisomer, mixture of stereoisomers, or solvate thereof, wherein:
A is C₁₋₆ alkylene, C₂₋₆ alkenylene, C₂₋₆ alkynylene, C₃₋₁₀ cycloalkylene, heterocyclylene, arylene, or heteroarylene; wherein the C₁₋₆ alkylene, C₂₋₆ alkenylene, C₂₋₆ alkynylene, C₃₋₁₀ cycloalkylene, heterocyclylene, arylene, or heteroaryl of A is independently optionally substituted with one to five Z^{A};
Ring B is a 5- or 6-membered heteroaryl optionally substituted with one to three R^{B};
each R^{B} is independently selected from halo, hydroxy, —NH₂, cyano, C₁₋₃ alkyl, C₁₋₃ haloalkyl, C₁₋₃ alkoxy, and C₁₋₃ haloalkoxy; wherein each C₁₋₃ alkyl of R^{B} is independently optionally substituted with —NH₂, —NHC₁₋₃ alkyl, —N(C₁₋₃ alkyl)₂, hydroxy, or C₁₋₃ alkoxy;
L¹ is C₁₋₃ alkylene, C₂₋₃ alkenylene, C₂₋₃ alkynylene, C₁₋₃ heteroalkylene, C₃₋₆ cycloalkylene, or 4-6 membered heterocyclylene; wherein the C₁₋₃ alkylene, C₂₋₃ alkenylene, C₂₋₃ alkynylene, C₁₋₃ heteroalkylene, C₃₋₆ cycloalkylene, or 4-6 membered heterocyclylene of L¹ is independently optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
L² is a bond, —O—, —S—, —NR^{2a}—, —C(O)—, —C(O)O—, —OC(O)—, —OC(O)O—, —C(O)NR^{2a}—, —NR^{2a}C(O)—, —OC(O)NR^{2a}—, —NR^{2a}C(O)O—, —NR^{2a}C(O)NR^{2b}—, —S(O)—, —S(O)₂—, —S(O)NR^{2a}—, —S(O)₂NR^{2a}—, —NR^{2a}S(O)—, —NR^{2a}S(O)₂—, —NR^{2a}S(O)NR^{2b}—, —NR^{2a}S(O)₂NR^{2b}—, C₁₋₆ alkylene, C₂₋₆ alkenylene, C₂₋₆ alkynylene, C₁₋₆ heteroalkylene, C₃₋₆ cycloalkylene, 4-6 membered heterocyclylene, or 5 membered heteroarylene; wherein the C₁₋₃ alkylene, C₂₋₃ alkenylene, C₂₋₃ alkynylene, C₁₋₃ heteroalkylene, C₃₋₆ cycloalkylene, 4-6 membered heterocyclylene, or 5 membered heteroarylene of L² is independently optionally substituted with one to five substituents independently selected from halo, oxo, hydroxy, cyano, —NH₂, —NHC₁₋₃ alkyl, —N(C₁₋₃ alkyl)₂, C₁₋₃ alkyl, C₁₋₃ haloalkyl, C₁₋₃ alkoxy, and C₁₋₃ haloalkoxy;
R¹ is cyano, —C(O)NR^{1a}R^{1b}, —C(S)NR^{1a}R^{1b}, —S(O)₂R², —S(O)(NR⁶)R², or —P(O)R⁷R²;
R² is —NR^{1a}R^{1b}, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl of R² is optionally substituted with one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, or C₁₋₃ haloalkoxy;
or R² and R⁶, or R² and R⁷, together with the atom to which they are attached, form a heterocyclyl optionally substituted with one to five Z²;
each R^{1a} and R^{1b} is independently hydrogen, C₁₋₃ alkyl, C₂₋₃ alkenyl, C₂₋₃ alkynyl, C₁₋₃ haloalkyl, C₃₋₆ cycloalkyl, or 4-6 membered heterocyclyl; wherein each C₁₋₃ alkyl, C₂₋₃ alkenyl, C₂₋₃ alkynyl, C₁₋₃ haloalkyl, C₃₋₆ cycloalkyl, or 4-6 membered heterocyclyl of R^{1a} and R^{1b} is independently optionally substituted with one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, or C₁₋₃ haloalkoxy;
or R^{1a} and R^{1b} are taken together with the atoms to which they are attached to form 4-6 membered heterocyclyl independently optionally substituted by one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, or C₁₋₃ haloalkoxy;
each R^{2a} and R^{2b} is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{2a} and R^{2b} is independently optionally substituted with one to five Z^{2a};
or R^{2a} and R^{2b} are taken together with the atoms to which they are attached to form heterocyclyl independently optionally substituted by one to five Z^{2a};
R³ is hydrogen, —NR^{3b}R^{3c}, C₁₋₆ alkyl, C₂₋₆ alkenyl, C₂₋₆ alkynyl, C₁₋₆ haloalkyl, C₁₋₆ alkoxy, C₁₋₆ haloalkoxy, C₁₋₆ heteroalkyl, C₁₋₆ haloalkyl, C₁₋₆ haloalkoxy, C₃₋₁₀ cycloalkyl, heterocyclyl, aryl, or heteroaryl; wherein the C₁₋₆ alkyl, C₂₋₆ alkenyl, C₂₋₆ alkynyl, C₁₋₆ haloalkyl, C₁₋₆ alkoxy, C₁₋₆ heteroalkyl, C₁₋₆ haloalkyl, C₁₋₆ haloalkoxy, C₃₋₁₀ cycloalkyl, heterocyclyl, aryl, or heteroaryl of R³ is independently optionally substituted with one to five Z³;
R^{3b} and R^{3c} are each independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{3b} and R^{3c} is independently optionally substituted with one to five substituents independently selected from halo, hydroxy, cyano, C₁₋₃ alkyl, C₁₋₃ haloalkyl, C₁₋₃ alkoxy, and C₁₋₃ haloalkoxy;
or R^{3b} and R^{3c} are taken together with the nitrogen atom to which they are attached to form a heterocyclyl optionally substituted with one to five Z^{3b};
R⁴ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl of R⁴ is independently optionally substituted with one to five Z⁴;
R⁵ is hydrogen, halo, hydroxy, amino, cyano, C₁₋₆ alkyl, C₁₋₆ alkoxy, C₁₋₆ haloalkyl, C₃₋₁₀ cycloalkyl, heterocyclyl, aryl, or heteroaryl; wherein the C₁₋₆ alkyl, C₁₋₆ alkoxy, C₁₋₆ haloalkyl, C₃₋₁₀ cycloalkyl, heterocyclyl, aryl, or heteroaryl of R⁵ is independently optionally substituted with one to five Z⁵;
R⁶ is hydrogen, C₁₋₃ alkyl, C₁₋₃ haloalkyl, C₃₋₆ cycloalkyl, or 4 to 6-membered heterocyclyl; wherein the C₁₋₃ alkyl, C₁₋₃ haloalkyl, C₃₋₆ cycloalkyl, or 4 to 6-membered heterocyclyl is optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
R⁷ is hydroxy, C₁₋₃ alkoxy, C₁₋₃ haloalkoxy, C₁₋₃ alkyl, or C₁₋₃ haloalkyl;
each Z^{A}, Z², Z^{2a}, Z³, Z^{3b}, Z⁴, and Z⁵; is independently halo, cyano, nitro, oxo, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-H, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{A}, Z², Z^{2a}, Z³, Z^{3b}, Z⁴, and Z⁵ are each independently optionally substituted with one to five Z^{1a};
each L is independently —O—, —S—, —NR²⁰—, —C(O)—, —C(O)O—, —OC(O)—, —OC(O)O—, —C(O)NR²⁰—, —NR²⁰C(O)—, —OC(O)NR²⁰—, —NR²⁰C(O)O—, —NR²⁰C(O)NR²¹—, —S(O)—, —S(O)₂—, —S(O)NR²⁰—, —S(O)₂NR²⁰—, —NR²⁰S(O)—, —NR²⁰S(O)₂—, —NR²⁰S(O)NR²¹—, or —NR²⁰S(O)₂NR²¹—;
each R²⁰ and R²¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R²⁰ and R²¹ is independently optionally substituted with one to five Z^{1a}; or an R²⁰ and R²¹ are taken together with the atoms to which they are attached to form heterocyclyl independently optionally substituted by one to five Z^{1a}; and
each Z^{1a} is independently halo, hydroxy, cyano, nitro, oxo, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{1a} is independently optionally substituted with one to five substituents independently selected from C₁₋₆ alkyl, oxo, halo, hydroxy, and cyano;
provided that when A is C₁₋₆ alkylene; then R⁵ is C₃₋₁₀ cycloalkyl, heterocyclyl, aryl, or heteroaryl; wherein the C₃₋₁₀ cycloalkyl, heterocyclyl, aryl, or heteroaryl is independently optionally substituted.

In some embodiments, provided is a compound of Formula I:

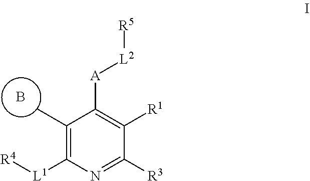

or a pharmaceutically acceptable salt, stereoisomer, mixture of stereoisomers, or solvate thereof, wherein:
A is C₁₋₆ alkylene, C₂₋₆ alkenylene, C₂₋₆ alkynylene, C₃₋₁₀ cycloalkylene, heterocyclylene, arylene, or heteroarylene; wherein the C₁₋₆ alkylene, C₂₋₆ alkenylene, C₂₋₆ alkynylene, C₃₋₁₀ cycloalkylene, heterocyclylene, arylene, or heteroarylene of A is independently optionally substituted with one to five Z^{A};
Ring B is a 5- or 6-membered heteroaryl optionally substituted with one to three R^{B};
each R^{B} is independently selected from halo, hydroxy, cyano, C₁₋₃ alkyl, C₁₋₃ haloalkyl, C₁₋₃ alkoxy, and C₁₋₃ haloalkoxy; wherein each C₁₋₃ alkyl of R^{B} is independently optionally substituted with —NH₂, —NHC₁₋₃ alkyl, —N(C₁₋₃ alkyl)₂, hydroxy, or C₁₋₃ alkoxy;
L¹ is C₁₋₃ alkylene, C₂₋₃ alkenylene, C₂₋₃ alkynylene, C₁₋₃ heteroalkylene, C₃₋₆ cycloalkylene, or 4-6 membered heterocyclylene; wherein the C₁₋₃ alkylene, C₂₋₃ alkenylene, C₂₋₃ alkynylene, C₁₋₃ heteroalkylene, C₃₋₆ cycloalkylene, or 4-6 membered heterocyclylene of L¹ is independently optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
L² is a bond, —O—, —S—, —NR^{2a}—, —C(O)—, —C(O)O—, —OC(O)—, —OC(O)O—, —C(O)NR^{2a}—, —NR^{2a}C(O)—, —OC(O)NR^{2a}—, —NR^{2a}C(O)O—, —NR^{2a}C(O)NR^{2b}—, —S(O)—, —S(O)₂—, —S(O)NR^{2a}—, —S(O)₂NR^{2a}—, —NR^{2a}S(O)—, —NR^{2a}S(O)₂—, —NR^{2a}S(O)NR^{2b}—, —NR^{2a}S(O)₂NR^{2b}—, C₁₋₆ alkylene, C₂₋₆ alkenylene, C₂₋₆ alkynylene, C₁₋₆ heteroalkylene, C₃₋₆ cycloalkylene, 4-6 membered heterocyclylene, or 5 membered heteroarylene; wherein the C₁₋₃ alkylene, C₂₋₃ alkenylene, C₂₋₃ alkynylene, C₁₋₃ heteroalkylene, C₃₋₆ cycloalkylene, 4-6 membered heterocyclylene, or 5 membered heteroarylene of L² is independently optionally substituted with one to five substituents independently selected from halo, oxo, hydroxy, cyano, —NH₂, —NHC₁₋₃ alkyl, —N(C₁₋₃ alkyl)₂, C₁₋₃ alkyl, C₁₋₃ haloalkyl, C₁₋₃ alkoxy, and C₁₋₃ haloalkoxy;
R¹ is cyano, —C(O)NR^{1a}R^{1b}, —C(S)NR^{1a}R^{1b}, —S(O)₂R², —S(O)(NR⁶)R², or —P(O)R⁷R²;
R² is —NR^{1a}R^{1b}, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl of R² is optionally substituted with one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, or C₁₋₃ haloalkoxy;
or R² and R⁶, or R² and R⁷, together with the atom to which they are attached, form a heterocyclyl optionally substituted with one to five Z²;
each R^{1a} and R^{1b} is independently hydrogen, C₁₋₃ alkyl, C₂₋₃ alkenyl, C₂₋₃ alkynyl, C₁₋₃ haloalkyl, C₃₋₆ cycloalkyl, or 4-6 membered heterocyclyl; wherein each C₁₋₃ alkyl, C₂₋₃ alkenyl, C₂₋₃ alkynyl, C₁₋₃ haloalkyl, C₃₋₆ cycloalkyl, or 4-6 membered heterocyclyl of R^{1a} and R^{1b} is independently optionally substituted with one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, or C₁₋₃ haloalkoxy;
or R^{1a} and R^{1b} are taken together with the atoms to which they are attached to form 4-6 membered heterocyclyl independently optionally substituted by one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, or C₁₋₃ haloalkoxy;
each R^{2a} and R^{2b} is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{2a} and R^{2b} is independently optionally substituted with one to five Z^{2a};
or R^{2a} and R^{2b} are taken together with the atoms to which they are attached to form heterocyclyl independently optionally substituted by one to five Z^{2a};
R³ is hydrogen, —NR^{3b}R^{3c}, C₁₋₆ alkyl, C₂₋₆ alkenyl, C₂₋₆ alkynyl, C₁₋₆ haloalkyl, C₁₋₆ alkoxy, C₁₋₆ haloalkoxy, C₁₋₆ heteroalkyl, C₁₋₆ haloalkyl, C₁₋₆ haloalkoxy, C₃₋₁₀ cycloalkyl, heterocyclyl, aryl, or heteroaryl; wherein the C₁₋₆ alkyl, C₂₋₆ alkenyl, C₂₋₆ alkynyl, C₁₋₆ haloalkyl, C₁₋₆ alkoxy, C₁₋₆ heteroalkyl, C₁₋₆ haloalkyl, C₁₋₆ haloalkoxy, C₃₋₁₀ cycloalkyl, heterocyclyl, aryl, or heteroaryl of R³ is independently optionally substituted with one to five Z³;
R^{3b} and R^{3c} are each independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{3b} and R^{3c} is independently optionally substituted with one to five substituents independently selected from halo, hydroxy, cyano, C₁₋₃ alkyl, C₁₋₃ haloalkyl, C₁₋₃ alkoxy, and C₁₋₃ haloalkoxy;
or R^{3b} and R^{3c} are taken together with the nitrogen atom to which they are attached to form a heterocyclyl optionally substituted with one to five Z^{3b};
R⁴ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl of R⁴ is independently optionally substituted with one to five Z⁴;
R⁵ is hydrogen, halo, hydroxy, amino, cyano, C₁₋₆ alkyl, C₁₋₆ alkoxy, C₁₋₆ haloalkyl, C₃₋₁₀ cycloalkyl, heterocyclyl, aryl, or heteroaryl; wherein the C₁₋₆ alkyl, C₁₋₆ alkoxy, C₁₋₆ haloalkyl, C₃₋₁₀ cycloalkyl, heterocyclyl, aryl, or heteroaryl of R⁵ is independently optionally substituted with one to five Z⁵;
R⁶ is hydrogen, C₁₋₃ alkyl, C₁₋₃ haloalkyl, C₃₋₆ cycloalkyl, or 4 to 6-membered heterocyclyl; wherein the C₁₋₃ alkyl, C₁₋₃ haloalkyl, C₃₋₆ cycloalkyl, or 4 to 6-membered heterocyclyl is optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
R⁷ is hydroxy, C₁₋₃ alkoxy, C₁₋₃ haloalkoxy, C₁₋₃ alkyl, or C₁₋₃ haloalkyl;
each Z^{A}, Z², Z^{2a}, Z³, Z^{3b}, Z⁴, and Z⁵; is independently halo, cyano, nitro, oxo, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-H, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{A}, Z², Z^{2a}, Z³, Z^{3b}, Z⁴, and Z⁵ are each independently optionally substituted with one to five Z^{1a};
each L is independently —O—, —S—, —NR²⁰—, —C(O)—, —C(O)O—, —OC(O)—, —OC(O)O—, —C(O)NR²⁰—, —NR²⁰C(O)—, —OC(O)NR²⁰—, —NR²⁰C(O)O—, —NR²⁰C(O)NR²¹—, —S(O)—, —S(O)₂—, —S(O)NR²⁰—, —S(O)₂NR²⁰—, —NR²⁰S(O)—, —NR²⁰S(O)₂—, —NR²⁰S(O)NR²¹—, or —NR²⁰S(O)₂NR²¹—;
each R²⁰ and R²¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R²⁰ and R²¹ is independently optionally substituted with one to five Z^{1a}; or an R²⁰ and R²¹ are taken together with the atoms to which they are attached to form heterocyclyl independently optionally substituted by one to five Z^{1a}; and
each Z^{1a} is independently halo, hydroxy, cyano, nitro, oxo, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{1a} is independently optionally substituted with one to five substituents independently selected from C₁₋₆ alkyl, oxo, halo, hydroxy, and cyano;
provided that when A is C₁₋₆ alkylene; then R⁵ is C₃₋₁₀ cycloalkyl, heterocyclyl, aryl, or heteroaryl; wherein the C₃₋₁₀ cycloalkyl, heterocyclyl, aryl, or heteroaryl is independently optionally substituted.

In some embodiments, R¹ is —C(O)NR^{1a}R^{1b}, —S(O)₂R², —S(O)(NR⁶)R², or —P(O)R⁷R².

In some embodiments, R² is —NR^{1a}R^{1b} or C₁₋₆ alkyl.

In some embodiments, R¹ is —C(O)NH₂, —S(O)₂NH₂, —S(O)₂CH₃,

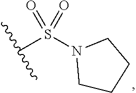 [SMILES: *S(=O)(=O)N1CCCC1]

—S(O)(NH)CH₃, or —P(O)(CH₃)CH₃.

In some embodiments, provided is a compound of Formula I:

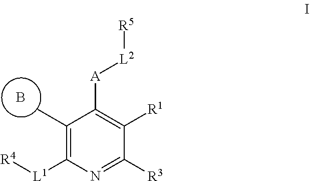

or a pharmaceutically acceptable salt, stereoisomer, mixture of stereoisomers, or solvate thereof, wherein:
A is 5- to 9-membered heteroarylene optionally substituted with one to five Z^{A};
Ring B is a 5- or 6-membered heteroaryl optionally substituted with one to three R^{B};
each R^{B} is independently selected from halo, hydroxy, cyano, C₁₋₃ alkyl, C₁₋₃ haloalkyl, C₁₋₃ alkoxy, and C₁₋₃ haloalkoxy; wherein each C₁₋₃ alkyl of R^{B} is independently optionally substituted with —NH₂, —NHC₁₋₃ alkyl, —N(C₁₋₃ alkyl)₂, hydroxy, or C₁₋₃ alkoxy;
L¹ is C₁₋₃ alkylene, C₂₋₃ alkenylene, C₂₋₃ alkynylene, C₁₋₃ heteroalkylene, C₃₋₆ cycloalkylene, or 4-6 membered heterocyclylene; wherein the C₁₋₃ alkylene, C₂₋₃ alkenylene, C₂₋₃ alkynylene, C₁₋₃ heteroalkylene, C₃₋₆ cycloalkylene, or 4-6 membered heterocyclylene of L¹ is independently optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
L² is a bond, —O—, —S—, —NR^{2a}—, —C(O)—, —C(O)O—, —OC(O)—, —OC(O)O—, —C(O)NR^{2a}—, —NR^{2a}C(O)—, —OC(O)NR^{2a}—, —NR^{2a}C(O)O—, —NR^{2a}C(O)NR^{2b}—, —S(O)—, —S(O)₂—, —S(O)NR^{2a}—, —S(O)₂NR^{2a}—, —NR^{2a}S(O)—, —NR^{2a}S(O)₂—, —NR^{2a}S(O)NR^{2b}—, —NR^{2a}S(O)₂NR^{2b}—, C₁₋₆ alkylene, C₂₋₆ alkenylene, C₂₋₆ alkynylene, C₁₋₆ heteroalkylene, C₃₋₆ cycloalkylene, 4-6 membered heterocyclylene, or 5 membered heteroarylene; wherein the C₁₋₃ alkylene, C₂₋₃ alkenylene, C₂₋₃ alkynylene, C₁₋₃ heteroalkylene, C₃₋₆ cycloalkylene, 4-6 membered heterocyclylene, or 5 membered heteroarylene of L² is independently optionally substituted with one to five substituents independently selected from halo, oxo, hydroxy, cyano, —NH₂, —NHC₁₋₃ alkyl, —N(C₁₋₃ alkyl)₂, C₁₋₃ alkyl, C₁₋₃ haloalkyl, C₁₋₃ alkoxy, and C₁₋₃ haloalkoxy;
R¹ is —C(O)NH₂;
each R^{2a} and R^{2b} is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{2a} and R^{2b} is independently optionally substituted with one to five Z^{2a};
or R^{2a} and R^{2b} are taken together with the atoms to which they are attached to form heterocyclyl independently optionally substituted by one to five Z^{2a};
R³ is C₁₋₆ alkyl optionally substituted with one to five Z³;
R⁴ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl of R⁴ is independently optionally substituted with one to five Z⁴;
R⁵ is C₃₋₁₀ cycloalkyl, heterocyclyl, aryl, or heteroaryl; wherein the C₃₋₁₀ cycloalkyl, heterocyclyl, aryl, or heteroaryl of R⁵ is independently optionally substituted with one to five Z⁵;
each Z^{A}, Z², Z^{2a}, Z³, Z^{3b}, Z⁴, and Z⁵; is independently halo, cyano, nitro, oxo, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-H, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{A}, Z², Z^{2a}, Z³, Z^{3b}, Z⁴, and Z⁵ are each independently optionally substituted with one to five Z^{1a};
each L is independently —O—, —S—, —NR²⁰—, —C(O)—, —C(O)O—, —OC(O)—, —OC(O)O—, —C(O)NR²⁰—, —NR²⁰C(O)—, —OC(O)NR²⁰—, —NR²⁰C(O)O—, —NR²⁰C(O)NR²¹—, —S(O)—, —S(O)₂—, —S(O)NR²⁰—, —S(O)₂NR²⁰—, —NR²⁰S(O)—, —NR²⁰S(O)₂—, —NR²⁰S(O)NR²¹—, or —NR²⁰S(O)₂NR²¹—;
each R²⁰ and R²¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R²⁰ and R²¹ is independently optionally substituted with one to five Z^{1a}; or an R²⁰ and R²¹ are taken together with the atoms to which they are attached to form heterocyclyl independently optionally substituted by one to five Z^{1a}; and each Z^{1a} is independently halo, hydroxy, cyano, nitro, oxo, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{1a} is independently optionally substituted with one to five substituents independently selected from C₁₋₆ alkyl, oxo, halo, hydroxy, and cyano.

In some embodiments, provided is a compound of Formula IA:

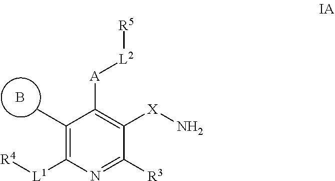

or a pharmaceutically acceptable salt, stereoisomer, mixture of stereoisomers, or solvate thereof, wherein A, L¹, L², R³, R⁴, R⁵ and Ring B are each independently as defined herein; and X is —C(O)— or —S(O)₂—;
provided that when A is C₁₋₆ alkylene; then R⁵ is C₃₋₁₀ cycloalkyl, heterocyclyl, aryl, or heteroaryl; wherein the C₃₋₁₀ cycloalkyl, heterocyclyl, aryl, or heteroaryl is independently optionally substituted.

In some embodiments of Formula IA, A is C₁₋₆ alkylene, C₂₋₆ alkenylene, C₂₋₆ alkynylene, C₃₋₁₀ cycloalkylene, heterocyclylene, arylene, or heteroarylene; wherein the C₁₋₆ alkylene, C₂₋₆ alkenylene, C₂₋₆ alkynylene, C₃₋₁₀ cycloalkylene, heterocyclylene, arylene, or heteroarylene of A is independently optionally substituted with one to five Z^{A};
Ring B is a 5- or 6-membered heteroaryl optionally substituted with one to three R^{B};
each R^{B} is independently selected from halo, hydroxy, cyano, C₁₋₃ alkyl, C₁₋₃ haloalkyl, C₁₋₃ alkoxy, and C₁₋₃ haloalkoxy; wherein each C₁₋₃ alkyl of R^{B} is independently optionally substituted with —NH₂, —NHC₁₋₃ alkyl, —N(C₁₋₃ alkyl)₂, hydroxy, or C₁₋₃ alkoxy;
L¹ is C₁₋₃ alkylene, C₂₋₃ alkenylene, C₂₋₃ alkynylene, C₁₋₃ heteroalkylene, C₃₋₆ cycloalkylene, or 4-6 membered heterocyclylene; wherein the C₁₋₃ alkylene, C₂₋₃ alkenylene, C₂₋₃ alkynylene, C₁₋₃ heteroalkylene, C₃₋₆ cycloalkylene, or 4-6 membered heterocyclylene of L¹ is independently optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
L² is a bond, —O—, —S—, —NR^{2a}—, —C(O)—, —C(O)O—, —OC(O)—, —OC(O)O—, —C(O)NR^{2a}—, —NR^{2a}C(O)—, —OC(O)NR^{2a}—, —NR^{2a}C(O)O—, —NR^{2a}C(O)NR^{2b}—, —S(O)—, —S(O)₂—, —S(O)NR^{2a}—, —S(O)₂NR^{2a}—, —NR^{2a}S(O)—, —NR^{2a}S(O)₂—, —NR^{2a}S(O)NR^{2b}—, —NR^{2a}S(O)₂NR^{2b}—, C₁₋₆ alkylene, C₂₋₆ alkenylene, C₂₋₆ alkynylene, C₁₋₆ heteroalkylene, C₃₋₆ cycloalkylene, 4-6 membered heterocyclylene, or 5 membered heteroarylene; wherein the C₁₋₃ alkylene, C₂₋₃ alkenylene, C₂₋₃ alkynylene, C₁₋₃ heteroalkylene, C₃₋₆ cycloalkylene, 4-6 membered heterocyclylene, or 5 membered heteroarylene of L² is independently optionally substituted with one to five substituents independently selected from halo, oxo, hydroxy, cyano, —NH₂, —NHC₁₋₃ alkyl, —N(C₁₋₃ alkyl)₂, C₁₋₃ alkyl, C₁₋₃ haloalkyl, C₁₋₃ alkoxy, and C₁₋₃ haloalkoxy;
each R^{2a} and R^{2b} is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{2a} and R^{2b} is independently optionally substituted with one to five Z^{2a};
or R^{2a} and R^{2b} are taken together with the atoms to which they are attached to form heterocyclyl independently optionally substituted by one to five Z^{2a};
R³ is hydrogen, —NR^{3b}R^{3c}, C₁₋₆ alkyl, C₂₋₆ alkenyl, C₂₋₆ alkynyl, C₁₋₆ haloalkyl, C₁₋₆ alkoxy, C₁₋₆ haloalkoxy, C₁₋₆ heteroalkyl, C₁₋₆ haloalkyl, C₁₋₆ haloalkoxy, C₃₋₁₀ cycloalkyl, heterocyclyl, aryl, or heteroaryl; wherein the C₁₋₆ alkyl, C₂₋₆ alkenyl, C₂₋₆ alkynyl, C₁₋₆ haloalkyl, C₁₋₆ alkoxy, C₁₋₆ heteroalkyl, C₁₋₆ haloalkyl, C₁₋₆ haloalkoxy, C₃₋₁₀ cycloalkyl, heterocyclyl, aryl, or heteroaryl of R³ is independently optionally substituted with one to five Z³;
R^{3b} and R^{3c} are each independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{3b} and R^{3c} is independently optionally substituted with one to five substituents independently selected from halo, hydroxy, cyano, C₁₋₃ alkyl, C₁₋₃ haloalkyl, C₁₋₃ alkoxy, and C₁₋₃ haloalkoxy;
or R^{3b} and R^{3c} are taken together with the nitrogen atom to which they are attached to form a heterocyclyl optionally substituted with one to five Z^{3b};
R⁴ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl of R⁴ is independently optionally substituted with one to five Z⁴;
R⁵ is hydrogen, halo, hydroxy, amino, cyano, C₁₋₆ alkyl, C₁₋₆ alkoxy, C₁₋₆ haloalkyl, C₃₋₁₀ cycloalkyl, heterocyclyl, aryl, or heteroaryl; wherein the C₁₋₆ alkyl, C₁₋₆ alkoxy, C₁₋₆ haloalkyl, C₃₋₁₀ cycloalkyl, heterocyclyl, aryl, or heteroaryl of R⁵ is independently optionally substituted with one to five Z⁵;
each Z^{A}, Z^{2a}, Z³, Z^{3b}, Z⁴, and Z⁵; is independently halo, cyano, nitro, oxo, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-H, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{A}, Z^{2a}, Z³, Z^{3b}, Z⁴, and Z⁵ are each independently optionally substituted with one to five Z¹a;
each L is independently —O—, —S—, —NR²⁰—, —C(O)—, —C(O)O—, —OC(O)—, —OC(O)O—, —C(O)NR²⁰—, —NR²⁰C(O)—, —OC(O)NR²⁰—, —NR²⁰C(O)O—, —NR²⁰C(O)NR²¹—, —S(O)—, —S(O)₂—, —S(O)NR²⁰—, —S(O)₂NR²⁰—, —NR²⁰S(O)—, —NR²⁰S(O)₂—, —NR²⁰S(O)NR²¹—, or —NR²⁰S(O)₂NR²¹—;
each R²⁰ and R²¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R²⁰ and R²¹ is independently optionally substituted with one to five Z^{1a}; or an R²⁰ and R²¹ are taken together with the atoms to which they are attached to form heterocyclyl independently optionally substituted by one to five Z^{1a}; and
each Z^{1a} is independently halo, hydroxy, cyano, nitro, oxo, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{1a} is independently optionally substituted with one to five substituents independently selected from C₁₋₆ alkyl, oxo, halo, hydroxy, and cyano;
provided that when A is C₁₋₆ alkylene; then R⁵ is C₃₋₁₀ cycloalkyl, heterocyclyl, aryl, or heteroaryl; wherein the C₃₋₁₀ cycloalkyl, heterocyclyl, aryl, or heteroaryl is independently optionally substituted.

In some embodiments, A is C₁₋₆ alkylene, C₃₋₁₀ cycloalkylene, heterocyclylene, arylene, or heteroarylene; wherein the C₁₋₆ alkylene, C₃₋₁₀ cycloalkylene, heterocyclylene, arylene, or heteroarylene of A is independently optionally substituted with one to five Z^{A}.

In some embodiments, A is C₁₋₆ alkylene, C₃₋₁₀ cycloalkylene, 3 to 10-membered heterocyclylene, C₆₋₁₀ arylene, or 5 to 10-membered heteroarylene; wherein each is independently optionally substituted with one to five Z^{A}.

In some embodiments, A is methylene, ethylene, n-propylene,

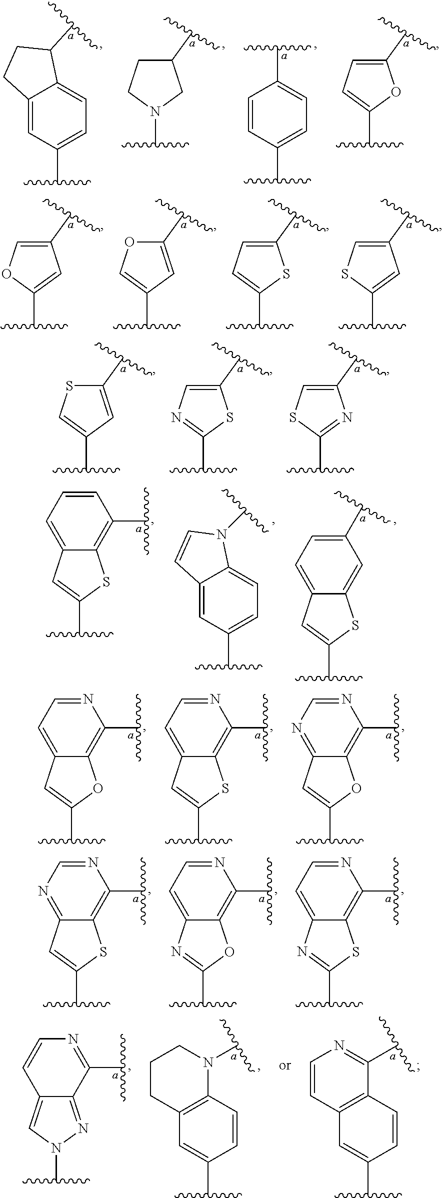

wherein bond a is bonded to L².

In some embodiments, A is methylene, ethylene, n-propylene,

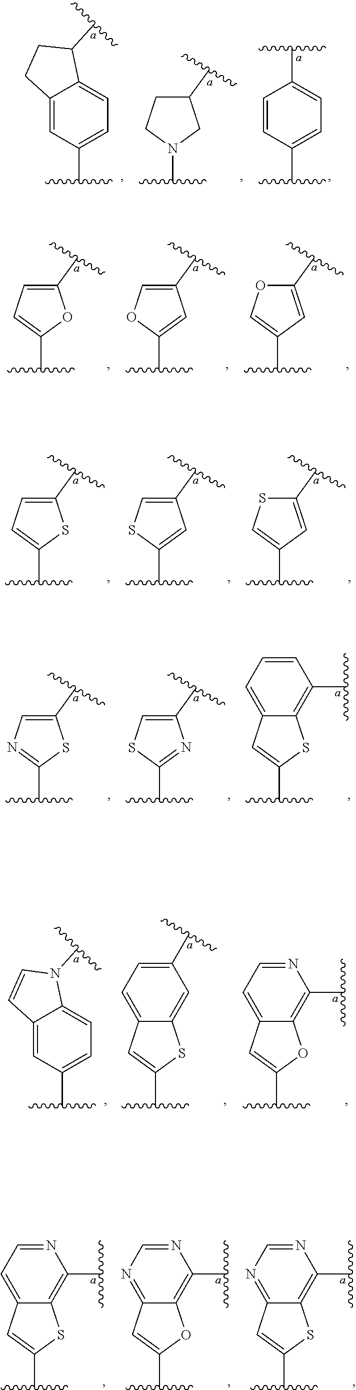

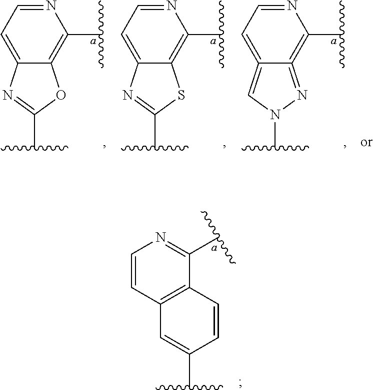

wherein bond a is bonded to L².

In some embodiments, A is

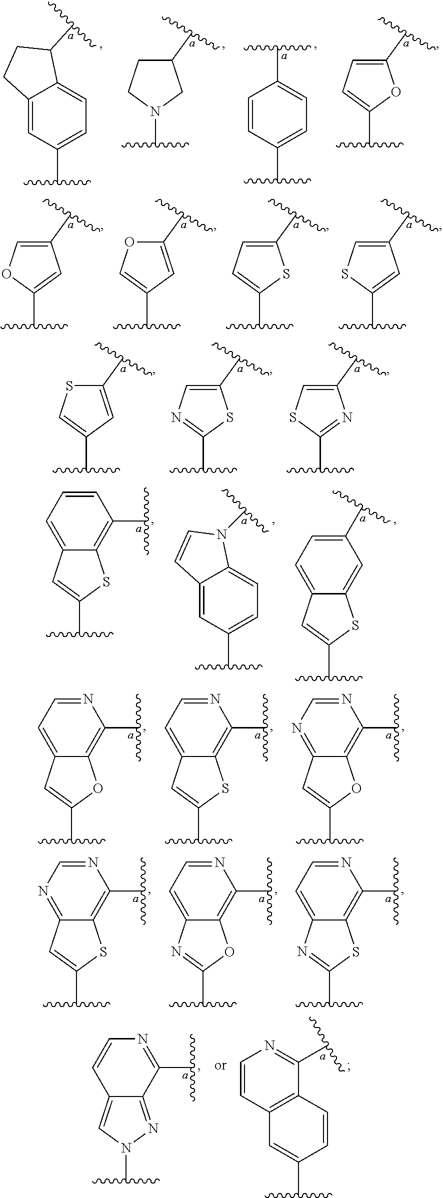

wherein bond a is bonded to L².

In some embodiments, provided is a compound of Formula IB:

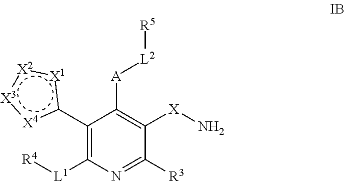

or a pharmaceutically acceptable salt, stereoisomer, mixture of stereoisomers, or solvate thereof, wherein A, L¹, L², R³, R⁴, and R⁵, are each independently as defined herein;
X is —C(O)— or —S(O)₂—; and
each of X¹, X², X³, and X⁴ are independently selected from O, S, N, NR^{B}, and CR^{B}; provided that at least one of X¹, X², X³, and X⁴ is N; and the ring encompassing X¹, X², X³, and X⁴ is aromatic; and
provided that when A is C₁₋₆ alkylene; then R⁵ is C₃₋₁₀ cycloalkyl, heterocyclyl, aryl, or heteroaryl; wherein the C₃₋₁₀ cycloalkyl, heterocyclyl, aryl, or heteroaryl is independently optionally substituted.

In some embodiments of Formula IB, A is C₁₋₆ alkylene, C₂₋₆ alkenylene, C₂₋₆ alkynylene, C₃₋₁₀ cycloalkylene, heterocyclylene, arylene, or heteroarylene; wherein the C₁₋₆ alkylene, C₂₋₆ alkenylene, C₂₋₆ alkynylene, C₃₋₁₀ cycloalkylene, heterocyclylene, arylene, or heteroarylene of A is independently optionally substituted with one to five Z^{A};
each R^{B} is independently selected from halo, hydroxy, cyano, C₁₋₃ alkyl, C₁₋₃ haloalkyl, C₁₋₃ alkoxy, and C₁₋₃ haloalkoxy; wherein each C₁₋₃ alkyl of R^{B} is independently optionally substituted with —NH₂, —NHC₁₋₃ alkyl, —N(C₁₋₃ alkyl)₂, hydroxy, or C₁₋₃ alkoxy;
L¹ is C₁₋₃ alkylene, C₂₋₃ alkenylene, C₂₋₃ alkynylene, C₁₋₃ heteroalkylene, C₃₋₆ cycloalkylene, or 4-6 membered heterocyclylene; wherein the C₁₋₃ alkylene, C₂₋₃ alkenylene, C₂₋₃ alkynylene, C₁₋₃ heteroalkylene, C₃₋₆ cycloalkylene, or 4-6 membered heterocyclylene of L¹ is independently optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
L² is a bond, —O—, —S—, —NR^{2a}—, —C(O)—, —C(O)O—, —OC(O)—, —OC(O)O—, —C(O)NR^{2a}—, —NR^{2a}C(O)—, —OC(O)NR^{2a}—, —NR^{2a}C(O)O—, —NR^{2a}C(O)NR^{2b}—, —S(O)—, —S(O)₂—, —S(O)NR^{2a}—, —S(O)₂NR^{2a}—, —NR^{2a}S(O)—, —NR^{2a}S(O)₂—, —NR^{2a}S(O)NR^{2b}—, —NR^{2a}S(O)₂NR^{2b}—, C₁₋₆ alkylene, C₂₋₆ alkenylene, C₂₋₆ alkynylene, C₁₋₆ heteroalkylene, C₃₋₆ cycloalkylene, 4-6 membered heterocyclylene, or 5 membered heteroarylene; wherein the C₁₋₃ alkylene, C₂₋₃ alkenylene, C₂₋₃ alkynylene, C₁₋₃ heteroalkylene, C₃₋₆ cycloalkylene, 4-6 membered heterocyclylene, or 5 membered heteroarylene of L² is independently optionally substituted with one to five substituents independently selected from halo, oxo, hydroxy, cyano, —NH₂, —NHC₁₋₃ alkyl, —N(C₁₋₃ alkyl)₂, C₁₋₃ alkyl, C₁₋₃ haloalkyl, C₁₋₃ alkoxy, and C₁₋₃ haloalkoxy;
each R^{2a} and R^{2b} is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{2a} and R^{2b} is independently optionally substituted with one to five Z^{2a};
or R^{2a} and R^{2b} are taken together with the atoms to which they are attached to form heterocyclyl independently optionally substituted by one to five Z^{2a};
R³ is hydrogen, —NR^{3b}R^{3c}, C₁₋₆ alkyl, C₂₋₆ alkenyl, C₂₋₆ alkynyl, C₁₋₆ haloalkyl, C₁₋₆ alkoxy, C₁₋₆ haloalkoxy, C₁₋₆ heteroalkyl, C₁₋₆ haloalkyl, C₁₋₆ haloalkoxy, C₃₋₁₀ cycloalkyl, heterocyclyl, aryl, or heteroaryl; wherein the C₁₋₆ alkyl, C₂₋₆ alkenyl, C₂₋₆ alkynyl, C₁₋₆ haloalkyl, C₁₋₆ alkoxy, C₁₋₆ heteroalkyl, C₁₋₆ haloalkyl, C₁₋₆ haloalkoxy, C₃₋₁₀ cycloalkyl, heterocyclyl, aryl, or heteroaryl of R³ is independently optionally substituted with one to five Z³;
R^{3b} and R^{3c} are each independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{3b} and R^{3c} is independently optionally substituted with one to five substituents independently selected from halo, hydroxy, cyano, C₁₋₃ alkyl, C₁₋₃ haloalkyl, C₁₋₃ alkoxy, and C₁₋₃ haloalkoxy;
or R^{3b} and R^{3c} are taken together with the nitrogen atom to which they are attached to form a heterocyclyl optionally substituted with one to five Z^{3b};
R⁴ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl of R⁴ is independently optionally substituted with one to five Z⁴;
R⁵ is hydrogen, halo, hydroxy, amino, cyano, C₁₋₆ alkyl, C₁₋₆ alkoxy, C₁₋₆ haloalkyl, C₃₋₁₀ cycloalkyl, heterocyclyl, aryl, or heteroaryl; wherein the C₁₋₆ alkyl, C₁₋₆ alkoxy, C₁₋₆ haloalkyl, C₃₋₁₀ cycloalkyl, heterocyclyl, aryl, or heteroaryl of R⁵ is independently optionally substituted with one to five Z⁵;
each Z^{A}, Z^{2a}, Z³, Z^{3b}, Z⁴, and Z⁵; is independently halo, cyano, nitro, oxo, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-H, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{A}, Z^{2a}, Z³, Z^{3b}, Z⁴, and Z⁵ are each independently optionally substituted with one to five Z¹a;
each L is independently —O—, —S—, —NR²⁰—, —C(O)—, —C(O)O—, —OC(O)—, —OC(O)O—, —C(O)NR²⁰—, —NR²⁰C(O)—, —OC(O)NR²⁰—, —NR²⁰C(O)O—, —NR²⁰C(O)NR²¹—, —S(O)—, —S(O)₂—, —S(O)NR²⁰—, —S(O)₂NR²⁰—, —NR²⁰S(O)—, —NR²⁰S(O)₂—, —NR²⁰S(O)NR²¹—, or —NR²⁰S(O)₂NR²¹—;
each R²⁰ and R²¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R²⁰ and R²¹ is independently optionally substituted with one to five Z^{1a}; or an R²⁰ and R²¹ are taken together with the atoms to which they are attached to form heterocyclyl independently optionally substituted by one to five Z^{1a}; and
each Z^{1a} is independently halo, hydroxy, cyano, nitro, oxo, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{1a} is independently optionally substituted with one to five substituents independently selected from C₁₋₆ alkyl, oxo, halo, hydroxy, and cyano;
provided that when A is C₁₋₆ alkylene; then R⁵ is C₃₋₁₀ cycloalkyl, heterocyclyl, aryl, or heteroaryl; wherein the C₃₋₁₀ cycloalkyl, heterocyclyl, aryl, or heteroaryl is independently optionally substituted.

In some embodiments, A is arylene or heteroarylene; wherein the arylene or heteroarylene is independently optionally substituted with one to five Z^{A}.

In some embodiments, A is arylene optionally substituted with one to five Z^{A}.

In some embodiments, A is heteroarylene optionally substituted with one to five Z^{A}.

In some embodiments, A is heterocyclylene optionally substituted with one to five Z^{A}.

In some embodiments, A is cycloalkylene optionally substituted with one to five Z^{A}.

In some embodiments, X is —C(O)—.

In some embodiments, X is —S(O)₂—.

In some embodiments, Ring B is a nitrogen-containing 5- or 6-membered heteroaryl optionally substituted with one to three R^{B}. The term “nitrogen-containing” is intended to refer to a heteroaryl which contains at least one ring nitrogen. The nitrogen-containing ring can contain one or more additional heteroatoms, such as oxygen, sulfur, or nitrogen).

In some embodiments, Ring B or the moiety

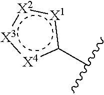

is a nitrogen-containing 5-membered heteroaryl optionally substituted with one to three R^{B}.

In some embodiments, Ring B or the moiety

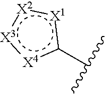

is pyrazolyl, isoxazolyl, oxadiazolyl, or thiadiazolyl; wherein the pyrazolyl, isoxazolyl, oxadiazolyl, or thiadiazolyl optionally substituted with one or two R^{B}.

In some embodiments, Ring B or the moiety

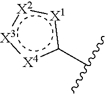

is:

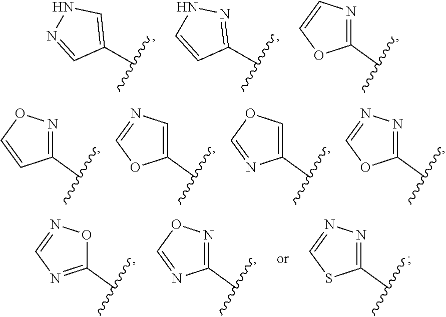

wherein each is optionally substituted with one or two R^{B}.

In some embodiments, each R^{B} is independently C₁₋₃ alkyl.

In some embodiments, Ring B is a 6-membered heteroaryl optionally substituted with one to three R^{B}.

In some embodiments, Ring B is pyridyl optionally substituted with one to three R^{B}.

In some embodiments, Ring B is pyridyl optionally substituted with C₁₋₃ alkyl.

In some embodiments, Ring B is pyridyl, pyrazolyl, isoxazolyl, oxadiazolyl, or thiadiazolyl; wherein the pyridyl, pyrazolyl, isoxazolyl, oxadiazolyl, or thiadiazolyl optionally substituted with one or two R^{B}.

In some embodiments, Ring B is:

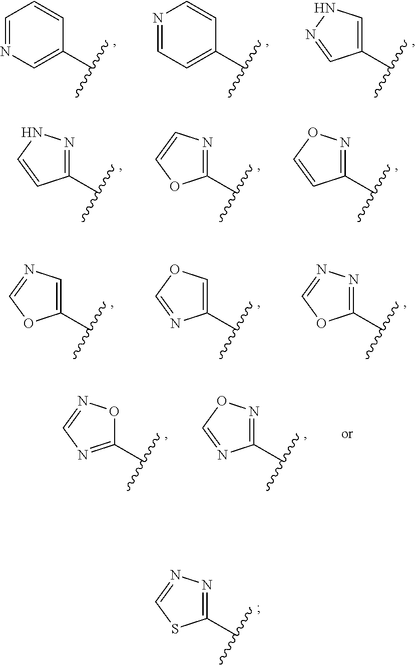

wherein each is optionally substituted with one or two R^{B}.

In some embodiments, L¹ is C₃₋₆cycloalkylene, C₁₋₃ alkylene or C₁₋₃ heteroalkylene; wherein each C₃₋₆ cycloalkylene, C₁₋₃ alkylene or C₁₋₃ heteroalkylene is independently optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy.

In some embodiments, L¹ is C₁₋₃ alkylene optionally substituted with one to three halo, oxo, hydroxy, C₁₋₃ alkyl, or C₁₋₃ alkoxy.

In some embodiments, L¹ is C₁₋₃ alkylene or C₁₋₃ heteroalkylene.

In some embodiments, R⁴ is C₁₋₆ alkyl, C₃₋₁₀ cycloalkyl, aryl, heterocyclyl, or heteroaryl; wherein the C₁₋₆ alkyl, C₃₋₁₀ cycloalkyl, aryl, heterocyclyl, or heteroaryl is independently optionally substituted with one to five Z⁴.

In some embodiments, R⁴ is C₁₋₆ alkyl, C₃₋₁₀ cycloalkyl, or aryl, or heteroaryl; wherein the C₁₋₆ alkyl, C₃₋₁₀ cycloalkyl, aryl, or heteroaryl is independently optionally substituted with one to five Z⁴.

In some embodiments, R⁴ is aryl, heterocyclyl, or heteroaryl; wherein the aryl, heterocyclyl, or heteroaryl is optionally substituted with one to five Z⁴.

In some embodiments, R⁴ is C₁₋₃ alkyl, C₃₋₉ cycloalkyl, 4-9 membered heterocyclyl, or phenyl; wherein the C₁₋₃ alkyl, C₃₋₆ cycloalkyl, or phenyl is independently optionally substituted with one to three halo.

In some embodiments, R⁴ is C₁₋₃ alkyl, C₃₋₆ cycloalkyl, or phenyl; wherein the C₁₋₃ alkyl, C₃₋₆ cycloalkyl, or phenyl is independently optionally substituted with one to three halo.

In some embodiments, R⁴ is C₁₋₃ alkyl, C₃₋₆ cycloalkyl, or phenyl; wherein the C₁₋₃ alkyl, C₃₋₆ cycloalkyl, or phenyl is independently optionally substituted with halo.

In some embodiments, R³ is —NR^{3b}R^{3c}, C₁₋₆ alkyl, C₂₋₆ alkenyl, C₂₋₆ alkynyl, C₁₋₆ haloalkyl, C₁₋₆ alkoxy, C₁₋₆ haloalkoxy, C₁₋₆ heteroalkyl, C₁₋₆ haloalkyl, C₁₋₆ haloalkoxy, C₃₋₁₀ cycloalkyl, heterocyclyl, aryl, or heteroaryl; wherein the C₁₋₆ alkyl, C₂₋₆ alkenyl, C₂₋₆ alkynyl, C₁₋₆ haloalkyl, C₁₋₆ alkoxy, C₁₋₆ heteroalkyl, C₁₋₆ haloalkyl, C₁₋₆ haloalkoxy, C₃₋₁₀ cycloalkyl, heterocyclyl, aryl, or heteroaryl of R³ is independently optionally substituted with one to five Z³.

In some embodiments, R³ is C₁₋₆ alkyl or C₁₋₆ haloalkyl.

In some embodiments, R³ is hydrogen, —NR^{3b}R^{3c}, C₁₋₆ alkyl, or C₁₋₆ alkoxy.

In some embodiments, L² is a bond, —NR^{2a}—, —C(O)NR^{2a}—, —NR^{2a}C(O)—, C₁₋₆ alkylene, C₂₋₆ alkenylene, C₂₋₆ alkynylene, C₁₋₆ heteroalkylene, 4-6 membered heterocyclylene, or 5 membered heteroarylene; wherein the C₁₋₆ alkylene, C₂₋₆ alkenylene, C₂₋₆ alkynylene, C₁₋₆ heteroalkylene, 4-6 membered heterocyclylene, or 5 membered heteroarylene is independently optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy.

In some embodiments, L² is a bond, —NR^{2a}—, —C(O)NR^{2a}—, —NR^{2a}C(O)—, C₁₋₆ alkylene, C₁₋₆ heteroalkylene, 4-6 membered heterocyclylene, or 5 membered heteroarylene; wherein the C₁₋₆ alkylene, C₁₋₆ heteroalkylene, 4-6 membered heterocyclylene, or 5 membered heteroarylene is independently optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy.

In some embodiments, L² is a bond, —C(O)—, —NH—, —NHCH₂—, —CH₂NH—, —OCH₂—, —CH₂O—, —C(O)NH—, —NHC(O)—, —C(O)NHCH₂—, —NHCH₂C(O)—, —OC(O)NHCH₂—, —CH₂NH(CO)O—, —CH₂CH₂—, or 1,2,3-triazoldiyl.

In some embodiments, R^{2a} is hydrogen.

In some embodiments, R⁵ is hydrogen, halo, amino, cyano, C₁₋₆ alkyl, C₁₋₆ alkoxy, C₃₋₁₀ cycloalkyl, heterocyclyl, aryl, or heteroaryl; wherein the C₁₋₆ alkyl, C₁₋₆ alkoxy, C₃₋₁₀ cycloalkyl, heterocyclyl, aryl, or heteroaryl is independently optionally substituted with one to five Z⁵.

In some embodiments, R⁵ is C₁₋₆ alkyl, aryl, or heteroaryl; wherein the aryl or heteroaryl is optionally substituted with one to five Z⁵.

In some embodiments, R⁵ is C₁₋₆ alkyl optionally substituted with C₃₋₁₀ cycloalkyl, heterocyclyl, aryl, or heteroaryl; wherein the C₃₋₁₀ cycloalkyl, heterocyclyl, aryl, or heteroaryl is optionally substituted with one to five Z^{1a}.

In some embodiments, R⁵ is aryl or heteroaryl; wherein the aryl or heteroaryl is optionally substituted with one to five Z⁵.

In some embodiments, R⁵ is hydrogen, halo, amino, cyano, C₁₋₆ alkyl, C₁₋₆ alkoxy, C₃₋₁₀ cycloalkyl, heterocyclyl, or aryl; wherein the C₁₋₆ alkyl, C₁₋₆ alkoxy, C₃₋₁₀ cycloalkyl, heterocyclyl, or aryl is independently optionally substituted with one to five Z⁵.

In some embodiments, R⁶ is C₁₋₃ alkyl or cyclopropyl. In some embodiments, R⁶ is C₁₋₃ alkyl.

In some embodiments, R⁷ is C₁₋₃ alkyl or cyclopropyl. In some embodiments, R⁷ is C₁₋₃ alkyl.

In some embodiments, provided is a compound of Formula IB:

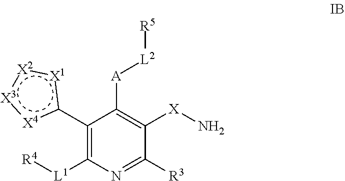

or a pharmaceutically acceptable salt, stereoisomer, mixture of stereoisomers, or solvate thereof, wherein A, L¹, and L² are each independently as defined herein;
X is —C(O)— or —S(O)₂—; and
each of X¹, X², X³, and X⁴ are independently selected from O, S, N, NR^{B}, and CR^{B}; provided that at least one of X¹, X², X³, and X⁴ is N; and the ring encompassing X¹, X², X³, and X⁴ is aromatic;
R³ is C₁₋₆ alkyl or C₁₋₆ haloalkyl;
R⁴ is aryl or heteroaryl; wherein each is optionally substituted with one to five Z⁴; and
R⁵ is aryl or heteroaryl; wherein each is optionally substituted with one to five Z⁵; and
provided that when A is C₁₋₆ alkylene; then R⁵ is C₃₋₁₀ cycloalkyl, heterocyclyl, aryl, or heteroaryl; wherein the C₃₋₁₀ cycloalkyl, heterocyclyl, aryl, or heteroaryl is independently optionally substituted.

In some embodiments of Formula IB, A is C₁₋₆ alkylene, C₂₋₆ alkenylene, C₂₋₆ alkynylene, C₃₋₁₀ cycloalkylene, heterocyclylene, arylene, or heteroarylene; wherein the C₁₋₆ alkylene, C₂₋₆ alkenylene, C₂₋₆ alkynylene, C₃₋₁₀ cycloalkylene, heterocyclylene, arylene, or heteroarylene of A is independently optionally substituted with one to five Z^{A};
each R^{B} is independently selected from halo, hydroxy, cyano, C₁₋₃ alkyl, C₁₋₃ haloalkyl, C₁₋₃ alkoxy, and C₁₋₃ haloalkoxy; wherein each C₁₋₃ alkyl of R^{B} is independently optionally substituted with —NH₂, —NHC₁₋₃ alkyl, —N(C₁₋₃ alkyl)₂, hydroxy, or C₁₋₃ alkoxy;
L¹ is C₁₋₃ alkylene, C₂₋₃ alkenylene, C₂₋₃ alkynylene, C₁₋₃ heteroalkylene, C₃₋₆ cycloalkylene, or 4-6 membered heterocyclylene; wherein the C₁₋₃ alkylene, C₂₋₃ alkenylene, C₂₋₃ alkynylene, C₁₋₃ heteroalkylene, C₃₋₆ cycloalkylene, or 4-6 membered heterocyclylene of L¹ is independently optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
L² is a bond, —O—, —S—, —NR^{2a}—, —C(O)—, —C(O)O—, —OC(O)—, —OC(O)O—, —C(O)NR^{2a}—, —NR^{2a}C(O)—, —OC(O)NR^{2a}—, —NR^{2a}C(O)O—, —NR^{2a}C(O)NR^{2b}—, —S(O)—, —S(O)₂—, —S(O)NR^{2a}—, —S(O)₂NR^{2a}—, —NR^{2a}S(O)—, —NR^{2a}S(O)₂—, —NR^{2a}S(O)NR^{2b}—, —NR^{2a}S(O)₂NR^{2b}—, C₁₋₆ alkylene, C₂₋₆ alkenylene, C₂₋₆ alkynylene, C₁₋₆ heteroalkylene, C₃₋₆ cycloalkylene, 4-6 membered heterocyclylene, or 5 membered heteroarylene; wherein the C₁₋₃ alkylene, C₂₋₃ alkenylene, C₂₋₃ alkynylene, C₁₋₃ heteroalkylene, C₃₋₆ cycloalkylene, 4-6 membered heterocyclylene, or 5 membered heteroarylene of L² is independently optionally substituted with one to five substituents independently selected from halo, oxo, hydroxy, cyano, —NH₂, —NHC₁₋₃ alkyl, —N(C₁₋₃ alkyl)₂, C₁₋₃ alkyl, C₁₋₃ haloalkyl, C₁₋₃ alkoxy, and C₁₋₃ haloalkoxy;
each R^{2a} and R^{2b} is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{2a} and R^{2b} is independently optionally substituted with one to five Z^{2a};
or R^{2a} and R^{2b} are taken together with the atoms to which they are attached to form heterocyclyl independently optionally substituted by one to five Z^{2a};
R³ is C₁₋₆ alkyl or C₁₋₆ haloalkyl;
R⁴ is aryl or heteroaryl; wherein each is optionally substituted with one to five Z⁵;
R⁵ is aryl or heteroaryl; wherein each is optionally substituted with one to five Z⁴;
each Z^{A}, Z^{2a}, Z⁴, and Z⁵; is independently halo, cyano, nitro, oxo, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-H, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{A}, Z^{2a}, Z⁴, and Z⁵ are each independently optionally substituted with one to five Z^{1a};
each L is independently —O—, —S—, —NR²⁰—, —C(O)—, —C(O)O—, —OC(O)—, —OC(O)O—, —C(O)NR²⁰—, —NR²⁰C(O)—, —OC(O)NR²⁰—, —NR²⁰C(O)O—, —NR²⁰C(O)NR²¹—, —S(O)—, —S(O)₂—, —S(O)NR²⁰—, —S(O)₂NR²⁰—, —NR²⁰S(O)—, —NR²⁰S(O)₂—, —NR²⁰S(O)NR²¹—, or —NR²⁰S(O)₂NR²¹—;
each R²⁰ and R²¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R²⁰ and R²¹ is independently optionally substituted with one to five Z^{1a}; or an R²⁰ and R²¹ are taken together with the atoms to which they are attached to form heterocyclyl independently optionally substituted by one to five Z^{1a}; and
each Z^{1a} is independently halo, hydroxy, cyano, nitro, oxo, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{1a} is independently optionally substituted with one to five substituents independently selected from C₁₋₆ alkyl, oxo, halo, hydroxy, and cyano;
provided that when A is C₁₋₆ alkylene; then R⁵ is C₃₋₁₀ cycloalkyl, heterocyclyl, aryl, or heteroaryl; wherein the C₃₋₁₀ cycloalkyl, heterocyclyl, aryl, or heteroaryl is independently optionally substituted.

In some embodiments, provided is a compound of Formula IC:

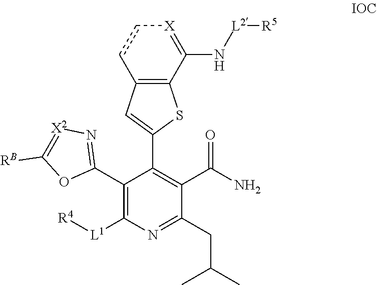

or a pharmaceutically acceptable salt, stereoisomer, mixture of stereoisomers, or solvate thereof, wherein A, L¹, and L² are each independently as defined herein;
X is O, and the dashed bonds are absent; or
X is N, and the dashed bonds are present;
X² is N or CH;
L^{2′} is a bond or —CH₂—;
R³ is C₁₋₆ alkyl or C₁₋₆ haloalkyl;
L¹ is C₁₋₃ alkylene or C₁₋₃ heteroalkylene; wherein the C₁₋₃ alkylene or C₁₋₃ heteroalkylene of L¹ is independently optionally substituted with halo, hydroxy, or C₁₋₃ alkyl;
R⁴ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl of R⁴ is independently optionally substituted with one or two halo, C₁₋₆ alkyl, or C₁₋₆ alkoxy;
R⁴ is aryl or heteroaryl; wherein each is optionally substituted with one to five Z⁴; and
R⁵ is C₃₋₁₀ cycloalkyl, heterocyclyl, aryl, or heteroaryl; wherein the C₃₋₁₀ cycloalkyl, heterocyclyl, aryl, or heteroaryl of R⁵ is independently optionally substituted with one to five halo, hydroxy, or C₁₋₆ alkoxy.

In some embodiments, provided is compound selected from Table 1, or a pharmaceutically acceptable salt, stereoisomer, mixture of stereoisomers, or solvate thereof:

TABLE 1

| Compound No. | Structure |
|---|---|
| 101 | 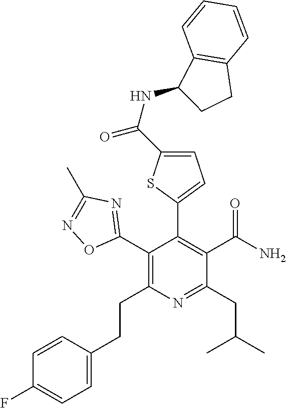 [SMILES: Cc1noc(-c2c(CCc3ccc(F)cc3)nc(CC(C)C)c(C(N)=O)c2-c2ccc(C(=O)N[C@@H]3CCc4ccccc43)s2)n1] |
| 102 | 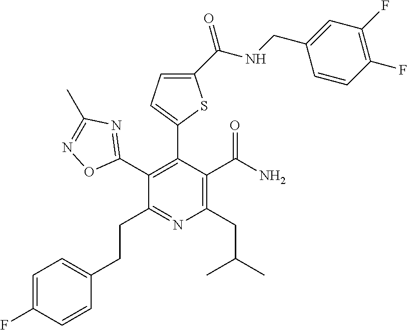 [SMILES: Cc1noc(-c2c(CCc3ccc(F)cc3)nc(CC(C)C)c(C(N)=O)c2-c2ccc(C(=O)NCc3ccc(F)c(F)c3)s2)n1] |
| 103 | 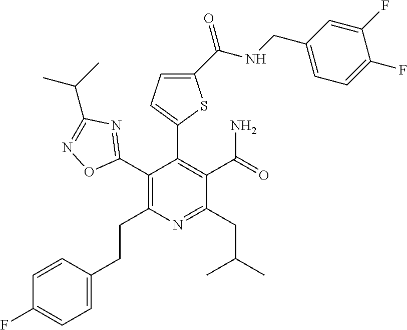 [SMILES: CC(C)Cc1nc(CCc2ccc(F)cc2)c(-c2nc(C(C)C)no2)c(-c2ccc(C(=O)NCc3ccc(F)c(F)c3)s2)c1C(N)=O] |
| 104 | 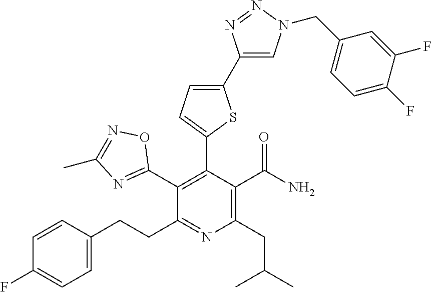 [SMILES: Cc1noc(-c2c(CCc3ccc(F)cc3)nc(CC(C)C)c(C(N)=O)c2-c2ccc(-c3cn(Cc4ccc(F)c(F)c4)nn3)s2)n1] |
| 105 | 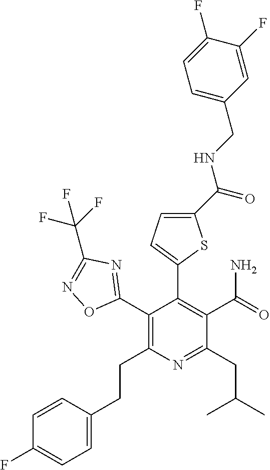 [SMILES: CC(C)Cc1nc(CCc2ccc(F)cc2)c(-c2nc(C(F)(F)F)no2)c(-c2ccc(C(=O)NCc3ccc(F)c(F)c3)s2)c1C(N)=O] |
| 106 | 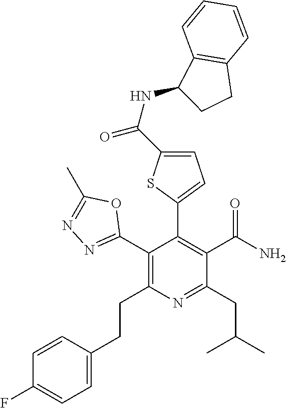 [SMILES: Cc1nnc(-c2c(CCc3ccc(F)cc3)nc(CC(C)C)c(C(N)=O)c2-c2ccc(C(=O)N[C@@H]3CCc4ccccc43)s2)o1] |
| 107 | 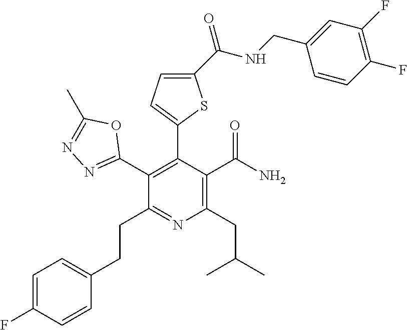 [SMILES: Cc1nnc(-c2c(CCc3ccc(F)cc3)nc(CC(C)C)c(C(N)=O)c2-c2ccc(C(=O)NCc3ccc(F)c(F)c3)s2)o1] |
| 108 | 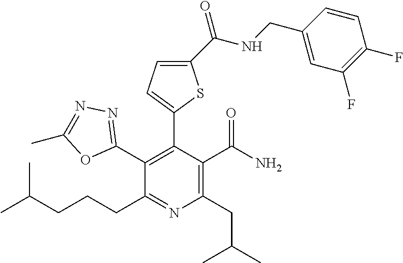 [SMILES: Cc1nnc(-c2c(CCCC(C)C)nc(CC(C)C)c(C(N)=O)c2-c2ccc(C(=O)NCc3ccc(F)c(F)c3)s2)o1] |
| 109 | 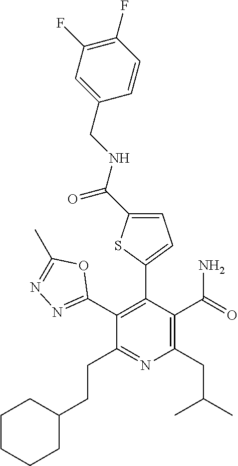 [SMILES: Cc1nnc(-c2c(CCC3CCCCC3)nc(CC(C)C)c(C(N)=O)c2-c2ccc(C(=O)NCc3ccc(F)c(F)c3)s2)o1] |
| 110 | 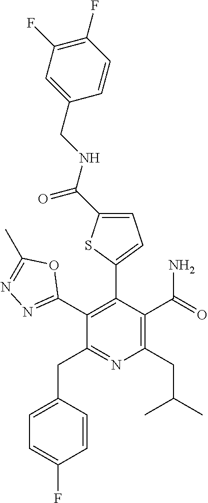 [SMILES: Cc1nnc(-c2c(Cc3ccc(F)cc3)nc(CC(C)C)c(C(N)=O)c2-c2ccc(C(=O)NCc3ccc(F)c(F)c3)s2)o1] |
| 111 | 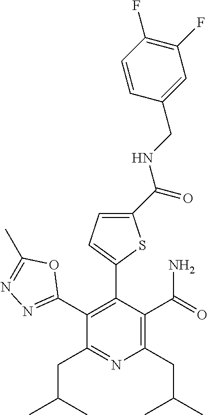 [SMILES: Cc1nnc(-c2c(CC(C)C)nc(CC(C)C)c(C(N)=O)c2-c2ccc(C(=O)NCc3ccc(F)c(F)c3)s2)o1] |
| 112 | 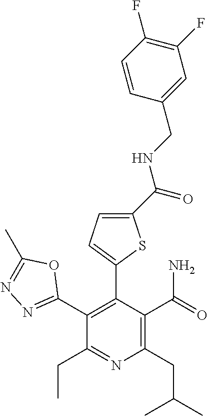 [SMILES: CCc1nc(CC(C)C)c(C(N)=O)c(-c2ccc(C(=O)NCc3ccc(F)c(F)c3)s2)c1-c1nnc(C)o1] |
| 113 | 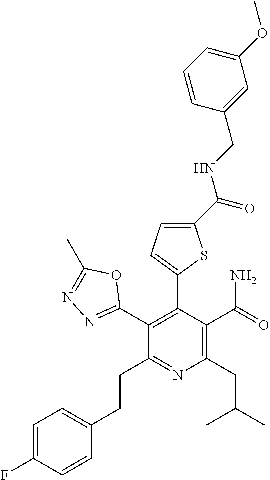 [SMILES: COc1cccc(CNC(=O)c2ccc(-c3c(C(N)=O)c(CC(C)C)nc(CCc4ccc(F)cc4)c3-c3nnc(C)o3)s2)c1] |
| 114 | 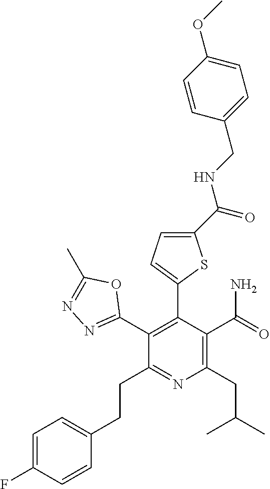 [SMILES: COc1ccc(CNC(=O)c2ccc(-c3c(C(N)=O)c(CC(C)C)nc(CCc4ccc(F)cc4)c3-c3nnc(C)o3)s2)cc1] |
| 115 | 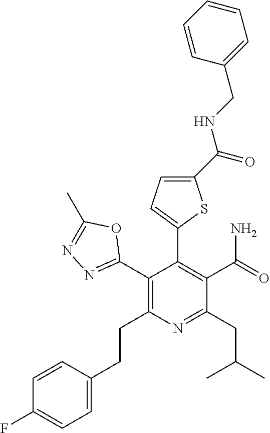 [SMILES: Cc1nnc(-c2c(CCc3ccc(F)cc3)nc(CC(C)C)c(C(N)=O)c2-c2ccc(C(=O)NCc3ccccc3)s2)o1] |
| 116 | 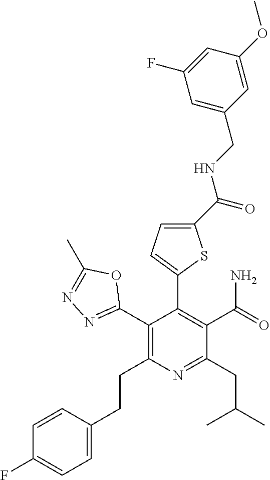 [SMILES: COc1cc(F)cc(CNC(=O)c2ccc(-c3c(C(N)=O)c(CC(C)C)nc(CCc4ccc(F)cc4)c3-c3nnc(C)o3)s2)c1] |
| 117 | 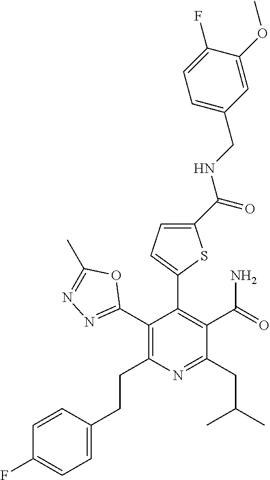 [SMILES: COc1cc(CNC(=O)c2ccc(-c3c(C(N)=O)c(CC(C)C)nc(CCc4ccc(F)cc4)c3-c3nnc(C)o3)s2)ccc1F] |
| 118 | 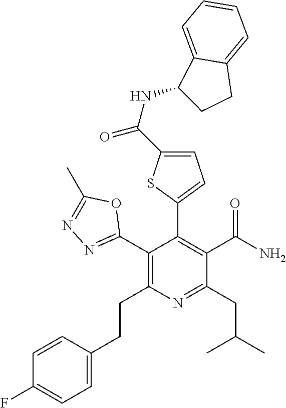 [SMILES: Cc1nnc(-c2c(CCc3ccc(F)cc3)nc(CC(C)C)c(C(N)=O)c2-c2ccc(C(=O)N[C@H]3CCc4ccccc43)s2)o1] |
| 119 | 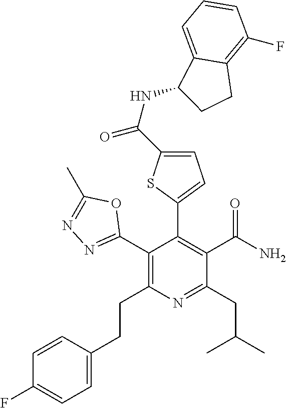 [SMILES: Cc1nnc(-c2c(CCc3ccc(F)cc3)nc(CC(C)C)c(C(N)=O)c2-c2ccc(C(=O)N[C@H]3CCc4c(F)cccc43)s2)o1] |
| 120 | 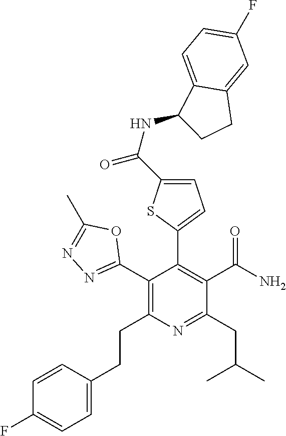 [SMILES: Cc1nnc(-c2c(CCc3ccc(F)cc3)nc(CC(C)C)c(C(N)=O)c2-c2ccc(C(=O)N[C@@H]3CCc4cc(F)ccc43)s2)o1] |
| 121 | 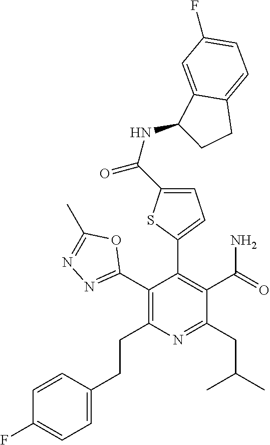 [SMILES: Cc1nnc(-c2c(CCc3ccc(F)cc3)nc(CC(C)C)c(C(N)=O)c2-c2ccc(C(=O)N[C@@H]3CCc4ccc(F)cc43)s2)o1] |
| 122 | 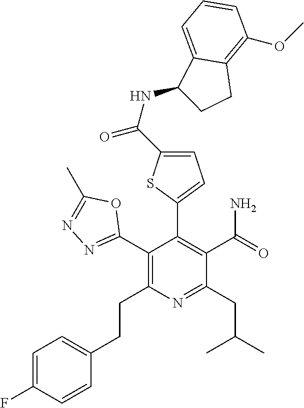 [SMILES: COc1cccc2c1CC[C@H]2NC(=O)c1ccc(-c2c(C(N)=O)c(CC(C)C)nc(CCc3ccc(F)cc3)c2-c2nnc(C)o2)s1] |
| 123 | 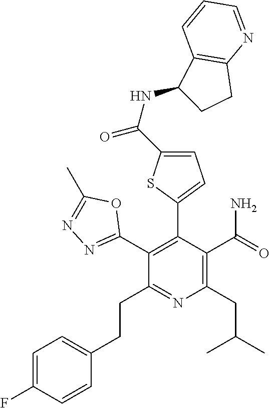 [SMILES: Cc1nnc(-c2c(CCc3ccc(F)cc3)nc(CC(C)C)c(C(N)=O)c2-c2ccc(C(=O)N[C@@H]3CCc4ncccc43)s2)o1] |
| 124 | 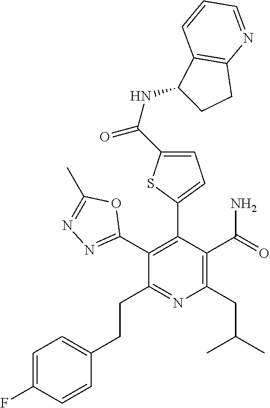 [SMILES: Cc1nnc(-c2c(CCc3ccc(F)cc3)nc(CC(C)C)c(C(N)=O)c2-c2ccc(C(=O)N[C@H]3CCc4ncccc43)s2)o1] |
| 125 | 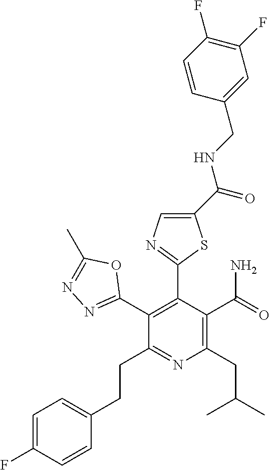 [SMILES: Cc1nnc(-c2c(CCc3ccc(F)cc3)nc(CC(C)C)c(C(N)=O)c2-c2ncc(C(=O)NCc3ccc(F)c(F)c3)s2)o1] |
| 126 | 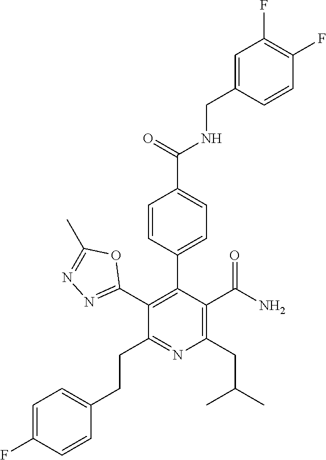 [SMILES: Cc1nnc(-c2c(CCc3ccc(F)cc3)nc(CC(C)C)c(C(N)=O)c2-c2ccc(C(=O)NCc3ccc(F)c(F)c3)cc2)o1] |
| 127 | 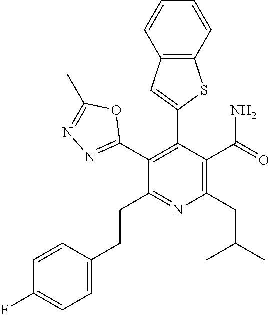 [SMILES: Cc1nnc(-c2c(CCc3ccc(F)cc3)nc(CC(C)C)c(C(N)=O)c2-c2cc3ccccc3s2)o1] |
| 128 | 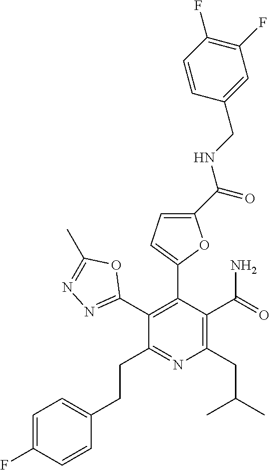 [SMILES: Cc1nnc(-c2c(CCc3ccc(F)cc3)nc(CC(C)C)c(C(N)=O)c2-c2ccc(C(=O)NCc3ccc(F)c(F)c3)o2)o1] |
| 129 | 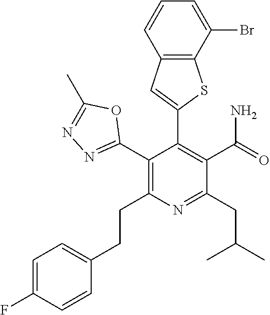 [SMILES: Cc1nnc(-c2c(CCc3ccc(F)cc3)nc(CC(C)C)c(C(N)=O)c2-c2cc3cccc(Br)c3s2)o1] |
| 130 | 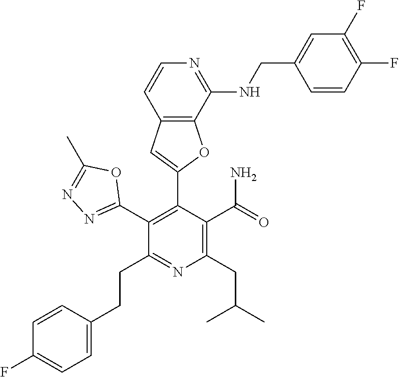 [SMILES: Cc1nnc(-c2c(CCc3ccc(F)cc3)nc(CC(C)C)c(C(N)=O)c2-c2cc3ccnc(NCc4ccc(F)c(F)c4)c3o2)o1] |
| 131 | 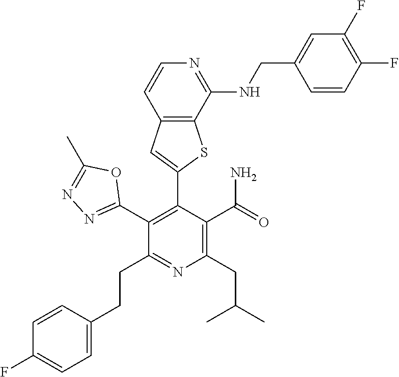 [SMILES: Cc1nnc(-c2c(CCc3ccc(F)cc3)nc(CC(C)C)c(C(N)=O)c2-c2cc3ccnc(NCc4ccc(F)c(F)c4)c3s2)o1] |
| 132 | 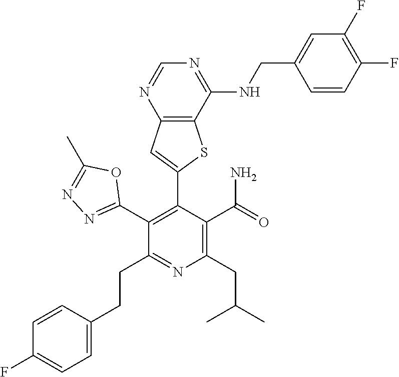 [SMILES: Cc1nnc(-c2c(CCc3ccc(F)cc3)nc(CC(C)C)c(C(N)=O)c2-c2cc3ncnc(NCc4ccc(F)c(F)c4)c3s2)o1] |
| 133 | 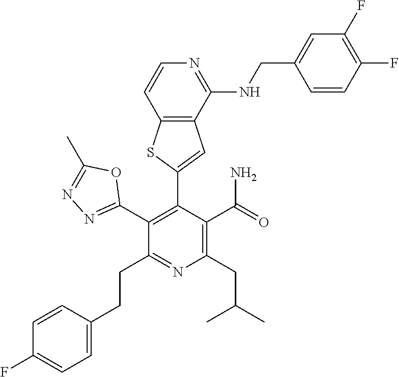 [SMILES: Cc1nnc(-c2c(CCc3ccc(F)cc3)nc(CC(C)C)c(C(N)=O)c2-c2cc3c(NCc4ccc(F)c(F)c4)nccc3s2)o1] |
| 134 | 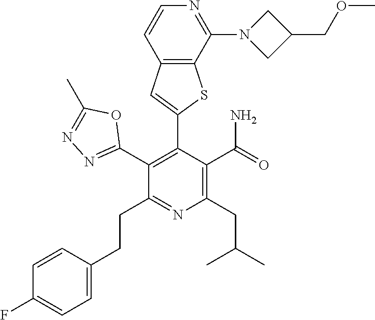 [SMILES: COCC1CN(c2nccc3cc(-c4c(C(N)=O)c(CC(C)C)nc(CCc5ccc(F)cc5)c4-c4nnc(C)o4)sc23)C1] |
| 135 | 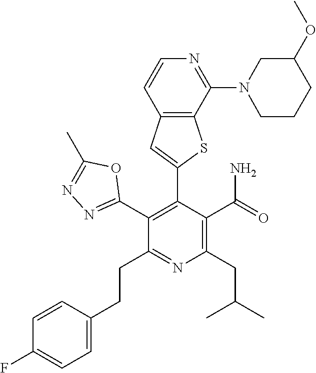 [SMILES: COC1CCCN(c2nccc3cc(-c4c(C(N)=O)c(CC(C)C)nc(CCc5ccc(F)cc5)c4-c4nnc(C)o4)sc23)C1] |
| 136 | 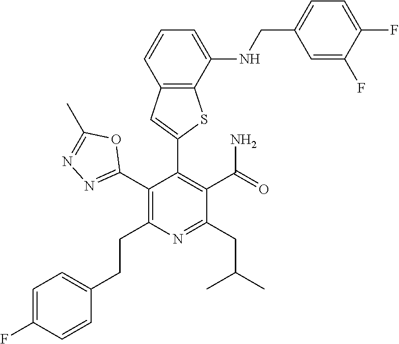 [SMILES: Cc1nnc(-c2c(CCc3ccc(F)cc3)nc(CC(C)C)c(C(N)=O)c2-c2cc3cccc(NCc4ccc(F)c(F)c4)c3s2)o1] |
| 137 | 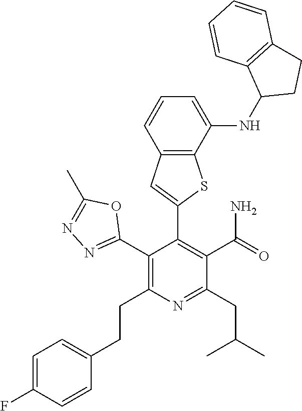 [SMILES: Cc1nnc(-c2c(CCc3ccc(F)cc3)nc(CC(C)C)c(C(N)=O)c2-c2cc3cccc(NC4CCc5ccccc54)c3s2)o1] |
| 138 | 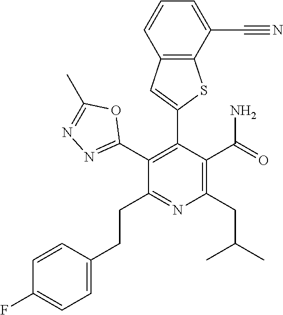 [SMILES: Cc1nnc(-c2c(CCc3ccc(F)cc3)nc(CC(C)C)c(C(N)=O)c2-c2cc3cccc(C#N)c3s2)o1] |
| 139 | 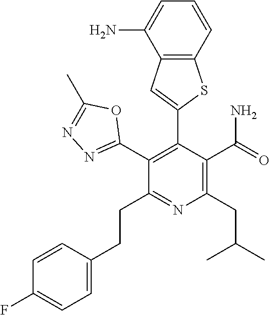 [SMILES: Cc1nnc(-c2c(CCc3ccc(F)cc3)nc(CC(C)C)c(C(N)=O)c2-c2cc3c(N)cccc3s2)o1] |
| 140 | 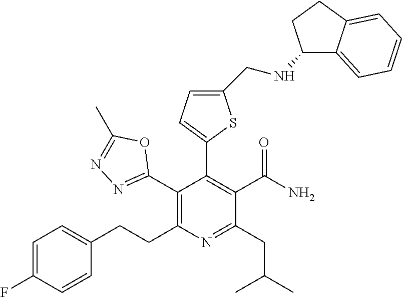 [SMILES: Cc1nnc(-c2c(CCc3ccc(F)cc3)nc(CC(C)C)c(C(N)=O)c2-c2ccc(CN[C@@H]3CCc4ccccc43)s2)o1] |
| 141 | 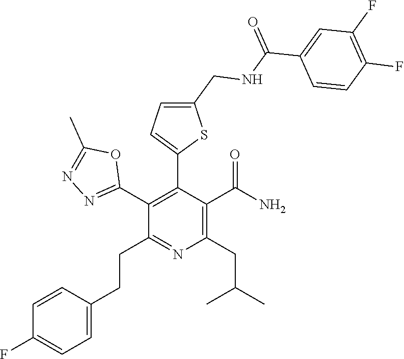 [SMILES: Cc1nnc(-c2c(CCc3ccc(F)cc3)nc(CC(C)C)c(C(N)=O)c2-c2ccc(CNC(=O)c3ccc(F)c(F)c3)s2)o1] |
| 142 | 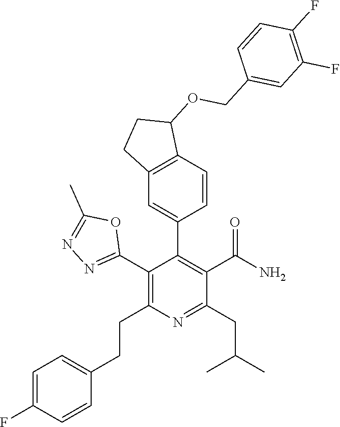 [SMILES: Cc1nnc(-c2c(CCc3ccc(F)cc3)nc(CC(C)C)c(C(N)=O)c2-c2ccc3c(c2)CCC3OCc2ccc(F)c(F)c2)o1] |
| 143 | 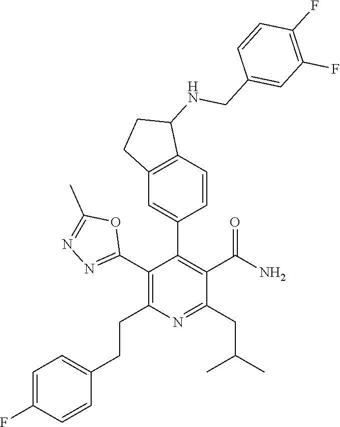 [SMILES: Cc1nnc(-c2c(CCc3ccc(F)cc3)nc(CC(C)C)c(C(N)=O)c2-c2ccc3c(c2)CCC3NCc2ccc(F)c(F)c2)o1] |
| 144 | 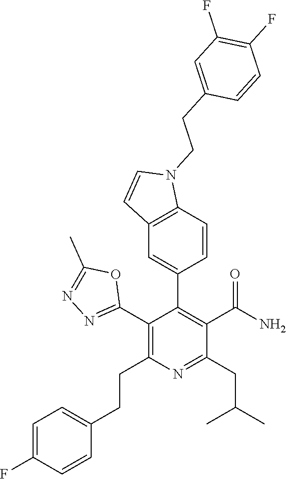 [SMILES: Cc1nnc(-c2c(CCc3ccc(F)cc3)nc(CC(C)C)c(C(N)=O)c2-c2ccc3c(ccn3CCc3ccc(F)c(F)c3)c2)o1] |
| 145 | 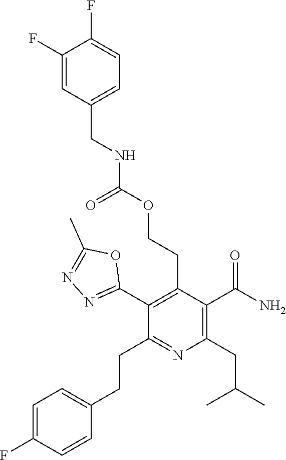 [SMILES: Cc1nnc(-c2c(CCc3ccc(F)cc3)nc(CC(C)C)c(C(N)=O)c2CCOC(=O)NCc2ccc(F)c(F)c2)o1] |
| 146 | 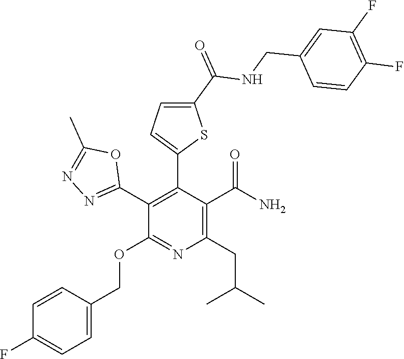 [SMILES: Cc1nnc(-c2c(OCc3ccc(F)cc3)nc(CC(C)C)c(C(N)=O)c2-c2ccc(C(=O)NCc3ccc(F)c(F)c3)s2)o1] |
| 147 | 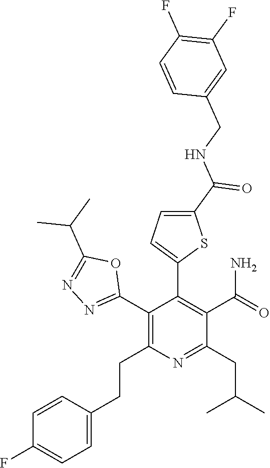 [SMILES: CC(C)Cc1nc(CCc2ccc(F)cc2)c(-c2nnc(C(C)C)o2)c(-c2ccc(C(=O)NCc3ccc(F)c(F)c3)s2)c1C(N)=O] |
| 148 | 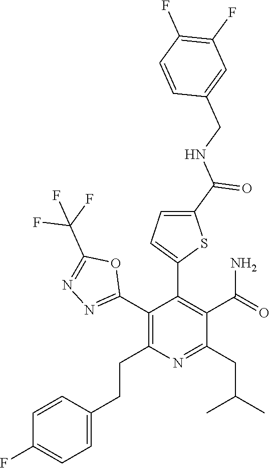 [SMILES: CC(C)Cc1nc(CCc2ccc(F)cc2)c(-c2nnc(C(F)(F)F)o2)c(-c2ccc(C(=O)NCc3ccc(F)c(F)c3)s2)c1C(N)=O] |
| 149 | 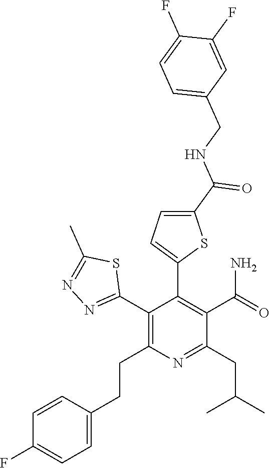 [SMILES: Cc1nnc(-c2c(CCc3ccc(F)cc3)nc(CC(C)C)c(C(N)=O)c2-c2ccc(C(=O)NCc3ccc(F)c(F)c3)s2)s1] |
| 150 | 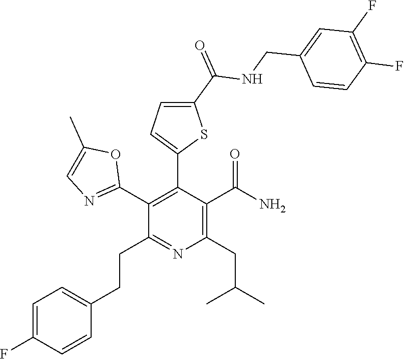 [SMILES: Cc1cnc(-c2c(CCc3ccc(F)cc3)nc(CC(C)C)c(C(N)=O)c2-c2ccc(C(=O)NCc3ccc(F)c(F)c3)s2)o1] |
| 151 | 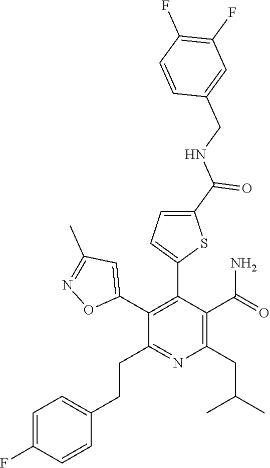 [SMILES: Cc1cc(-c2c(CCc3ccc(F)cc3)nc(CC(C)C)c(C(N)=O)c2-c2ccc(C(=O)NCc3ccc(F)c(F)c3)s2)on1] |
| 152 | 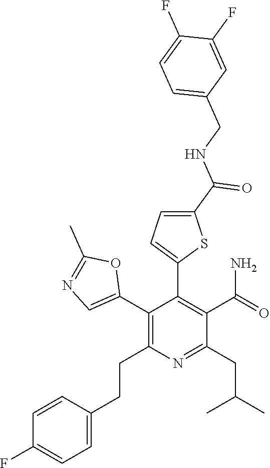 [SMILES: Cc1ncc(-c2c(CCc3ccc(F)cc3)nc(CC(C)C)c(C(N)=O)c2-c2ccc(C(=O)NCc3ccc(F)c(F)c3)s2)o1] |
| 153 | 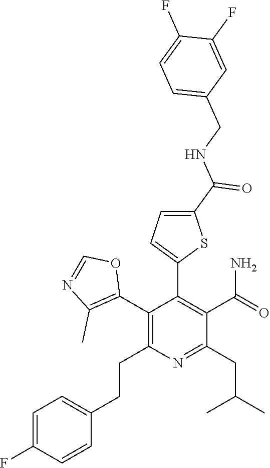 [SMILES: Cc1ncoc1-c1c(CCc2ccc(F)cc2)nc(CC(C)C)c(C(N)=O)c1-c1ccc(C(=O)NCc2ccc(F)c(F)c2)s1] |
| 154 | 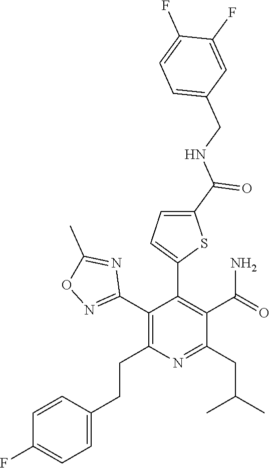 [SMILES: Cc1nc(-c2c(CCc3ccc(F)cc3)nc(CC(C)C)c(C(N)=O)c2-c2ccc(C(=O)NCc3ccc(F)c(F)c3)s2)no1] |
| 155 | 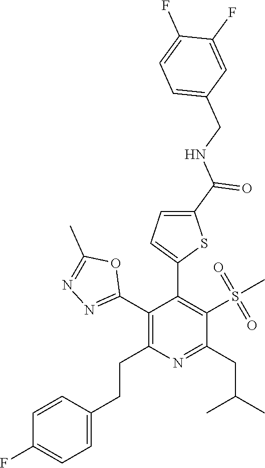 [SMILES: Cc1nnc(-c2c(CCc3ccc(F)cc3)nc(CC(C)C)c(S(C)(=O)=O)c2-c2ccc(C(=O)NCc3ccc(F)c(F)c3)s2)o1] |
| 156 | 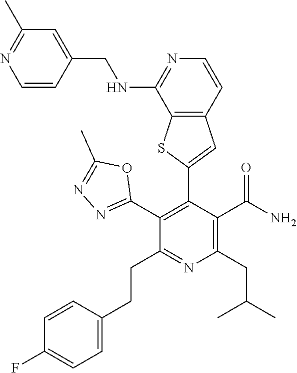 [SMILES: Cc1cc(CNc2nccc3cc(-c4c(C(N)=O)c(CC(C)C)nc(CCc5ccc(F)cc5)c4-c4nnc(C)o4)sc23)ccn1] |
| 157 | 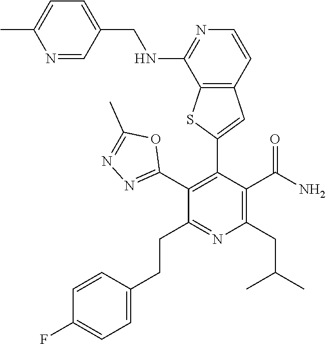 [SMILES: Cc1ccc(CNc2nccc3cc(-c4c(C(N)=O)c(CC(C)C)nc(CCc5ccc(F)cc5)c4-c4nnc(C)o4)sc23)cn1] |
| 158 | 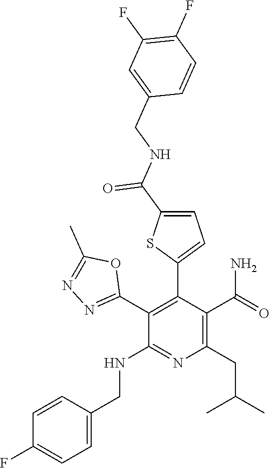 [SMILES: Cc1nnc(-c2c(NCc3ccc(F)cc3)nc(CC(C)C)c(C(N)=O)c2-c2ccc(C(=O)NCc3ccc(F)c(F)c3)s2)o1] |
| 159 | 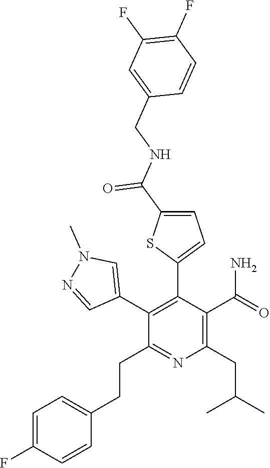 [SMILES: CC(C)Cc1nc(CCc2ccc(F)cc2)c(-c2cnn(C)c2)c(-c2ccc(C(=O)NCc3ccc(F)c(F)c3)s2)c1C(N)=O] |
| 160 | 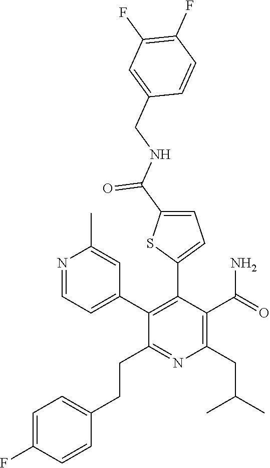 [SMILES: Cc1cc(-c2c(CCc3ccc(F)cc3)nc(CC(C)C)c(C(N)=O)c2-c2ccc(C(=O)NCc3ccc(F)c(F)c3)s2)ccn1] |
| 161 | 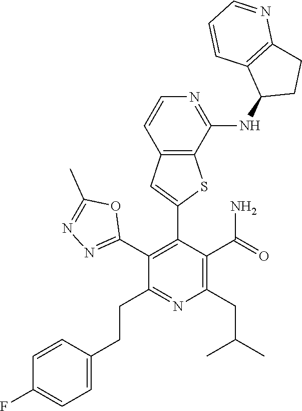 [SMILES: Cc1nnc(-c2c(CCc3ccc(F)cc3)nc(CC(C)C)c(C(N)=O)c2-c2cc3ccnc(N[C@@H]4CCc5ncccc54)c3s2)o1] |
| 162 | 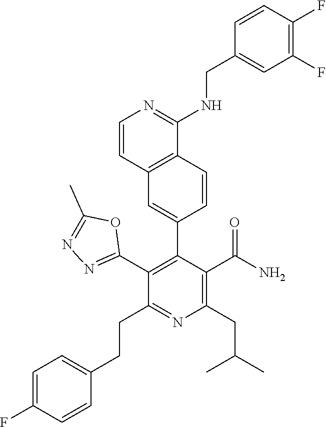 [SMILES: Cc1nnc(-c2c(CCc3ccc(F)cc3)nc(CC(C)C)c(C(N)=O)c2-c2ccc3c(NCc4ccc(F)c(F)c4)nccc3c2)o1] |
| 163 | 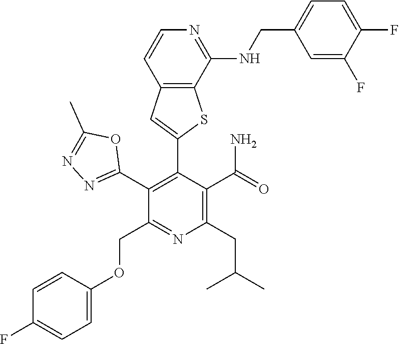 [SMILES: Cc1nnc(-c2c(COc3ccc(F)cc3)nc(CC(C)C)c(C(N)=O)c2-c2cc3ccnc(NCc4ccc(F)c(F)c4)c3s2)o1] |
| 164 | 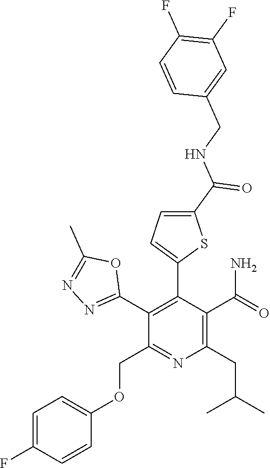 [SMILES: Cc1nnc(-c2c(COc3ccc(F)cc3)nc(CC(C)C)c(C(N)=O)c2-c2ccc(C(=O)NCc3ccc(F)c(F)c3)s2)o1] |
| 165 | 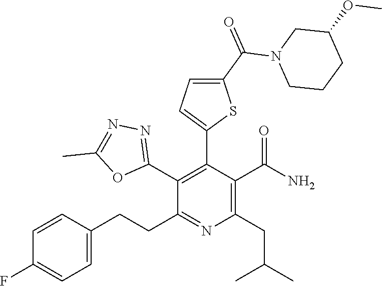 [SMILES: CO[C@@H]1CCCN(C(=O)c2ccc(-c3c(C(N)=O)c(CC(C)C)nc(CCc4ccc(F)cc4)c3-c3nnc(C)o3)s2)C1] |
| 166 | 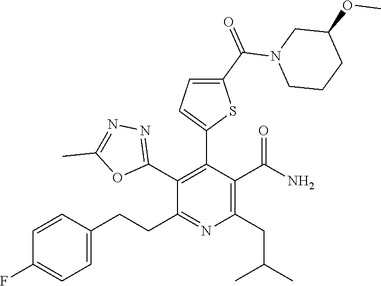 [SMILES: CO[C@H]1CCCN(C(=O)c2ccc(-c3c(C(N)=O)c(CC(C)C)nc(CCc4ccc(F)cc4)c3-c3nnc(C)o3)s2)C1] |
| 167 | 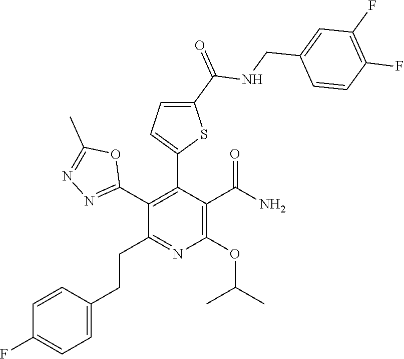 [SMILES: Cc1nnc(-c2c(CCc3ccc(F)cc3)nc(OC(C)C)c(C(N)=O)c2-c2ccc(C(=O)NCc3ccc(F)c(F)c3)s2)o1] |
| 168 | 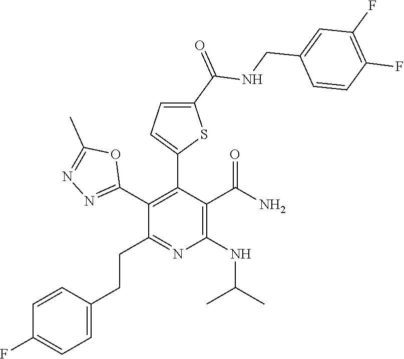 [SMILES: Cc1nnc(-c2c(CCc3ccc(F)cc3)nc(NC(C)C)c(C(N)=O)c2-c2ccc(C(=O)NCc3ccc(F)c(F)c3)s2)o1] |
| 169 | 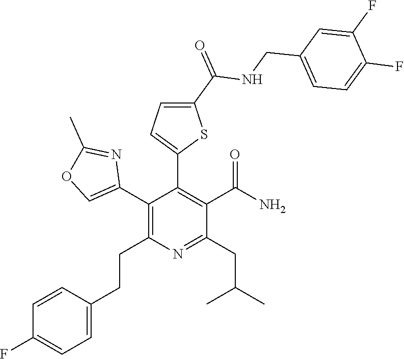 [SMILES: Cc1nc(-c2c(CCc3ccc(F)cc3)nc(CC(C)C)c(C(N)=O)c2-c2ccc(C(=O)NCc3ccc(F)c(F)c3)s2)co1] |
| 170 | 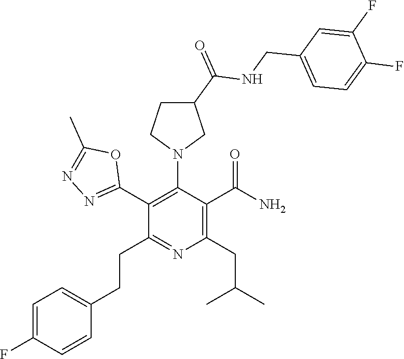 [SMILES: Cc1nnc(-c2c(CCc3ccc(F)cc3)nc(CC(C)C)c(C(N)=O)c2N2CCC(C(=O)NCc3ccc(F)c(F)c3)C2)o1] |
| 171 | 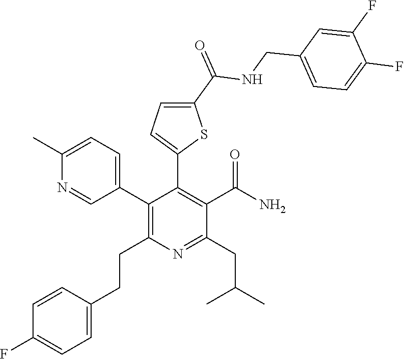 [SMILES: Cc1ccc(-c2c(CCc3ccc(F)cc3)nc(CC(C)C)c(C(N)=O)c2-c2ccc(C(=O)NCc3ccc(F)c(F)c3)s2)cn1] |
| 172 | 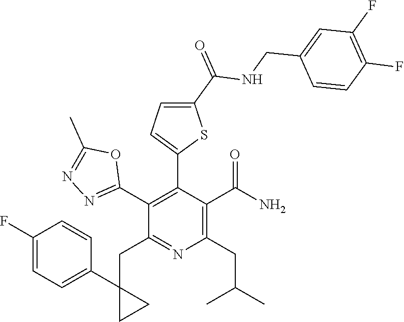 [SMILES: Cc1nnc(-c2c(CC3(c4ccc(F)cc4)CC3)nc(CC(C)C)c(C(N)=O)c2-c2ccc(C(=O)NCc3ccc(F)c(F)c3)s2)o1] |
| 173 | 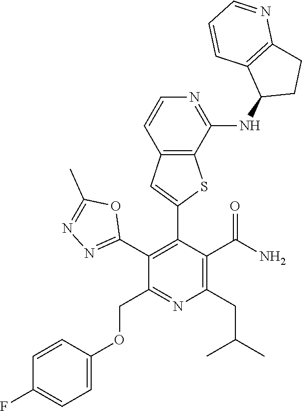 [SMILES: Cc1nnc(-c2c(COc3ccc(F)cc3)nc(CC(C)C)c(C(N)=O)c2-c2cc3ccnc(N[C@@H]4CCc5ncccc54)c3s2)o1] |
| 174 | 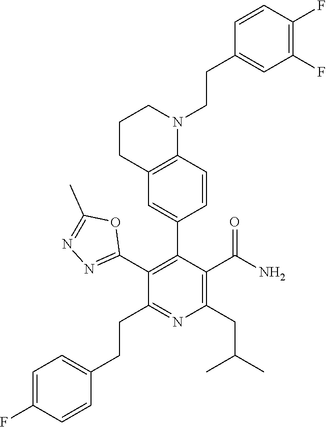 [SMILES: Cc1nnc(-c2c(CCc3ccc(F)cc3)nc(CC(C)C)c(C(N)=O)c2-c2ccc3c(c2)CCCN3CCc2ccc(F)c(F)c2)o1] |
| 175 | 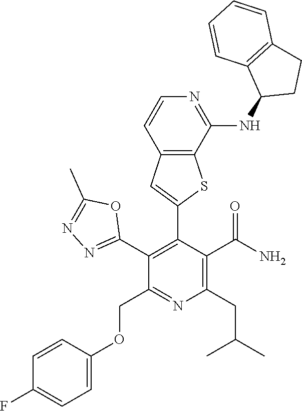 [SMILES: Cc1nnc(-c2c(COc3ccc(F)cc3)nc(CC(C)C)c(C(N)=O)c2-c2cc3ccnc(N[C@@H]4CCc5ccccc54)c3s2)o1] |
| 176 | 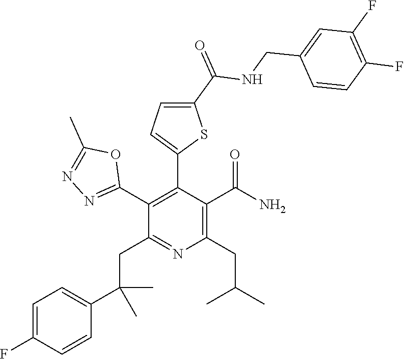 [SMILES: Cc1nnc(-c2c(CC(C)(C)c3ccc(F)cc3)nc(CC(C)C)c(C(N)=O)c2-c2ccc(C(=O)NCc3ccc(F)c(F)c3)s2)o1] |
| 177 | 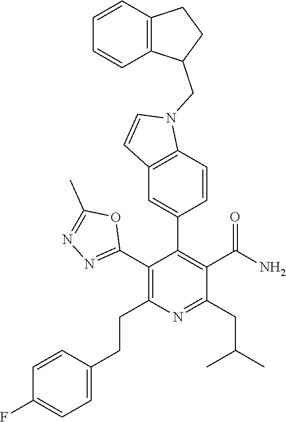 [SMILES: Cc1nnc(-c2c(CCc3ccc(F)cc3)nc(CC(C)C)c(C(N)=O)c2-c2ccc3c(ccn3CC3CCc4ccccc43)c2)o1] |
| 178 | 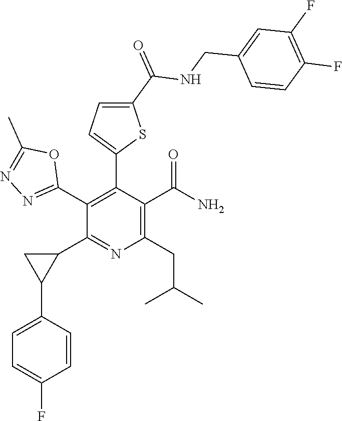 [SMILES: Cc1nnc(-c2c(C3CC3c3ccc(F)cc3)nc(CC(C)C)c(C(N)=O)c2-c2ccc(C(=O)NCc3ccc(F)c(F)c3)s2)o1] |
| 179 | 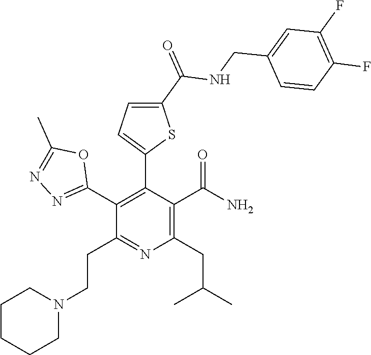 [SMILES: Cc1nnc(-c2c(CCN3CCCCC3)nc(CC(C)C)c(C(N)=O)c2-c2ccc(C(=O)NCc3ccc(F)c(F)c3)s2)o1] |
| 180 | 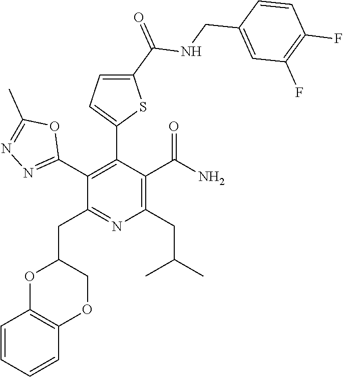 [SMILES: Cc1nnc(-c2c(CC3COc4ccccc4O3)nc(CC(C)C)c(C(N)=O)c2-c2ccc(C(=O)NCc3ccc(F)c(F)c3)s2)o1] |
| 181 | 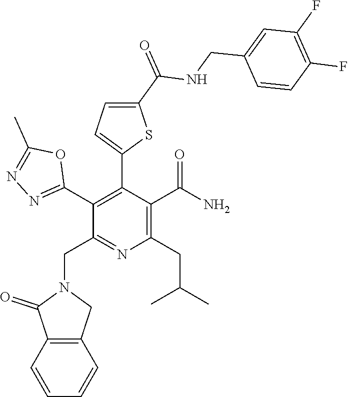 [SMILES: Cc1nnc(-c2c(CN3Cc4ccccc4C3=O)nc(CC(C)C)c(C(N)=O)c2-c2ccc(C(=O)NCc3ccc(F)c(F)c3)s2)o1] |
| 182 | 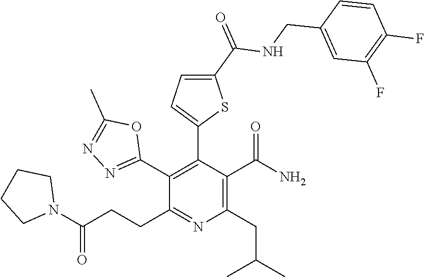 [SMILES: Cc1nnc(-c2c(CCC(=O)N3CCCC3)nc(CC(C)C)c(C(N)=O)c2-c2ccc(C(=O)NCc3ccc(F)c(F)c3)s2)o1] |
| 183 | 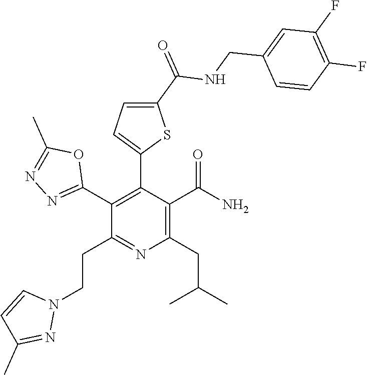 [SMILES: Cc1ccn(CCc2nc(CC(C)C)c(C(N)=O)c(-c3ccc(C(=O)NCc4ccc(F)c(F)c4)s3)c2-c2nnc(C)o2)n1] |
| 184 | 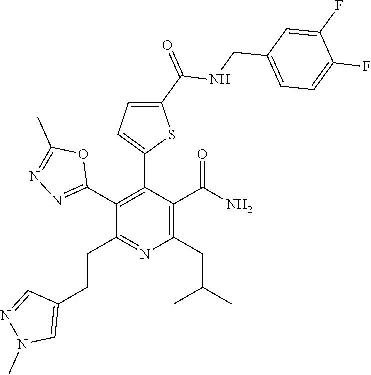 [SMILES: Cc1nnc(-c2c(CCc3cnn(C)c3)nc(CC(C)C)c(C(N)=O)c2-c2ccc(C(=O)NCc3ccc(F)c(F)c3)s2)o1] |
| 185 | 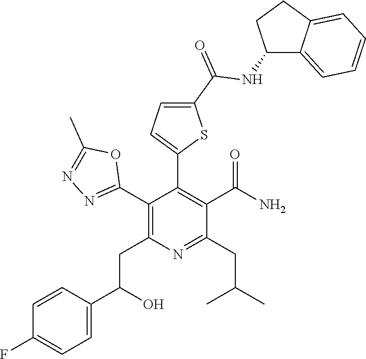 [SMILES: Cc1nnc(-c2c(CC(O)c3ccc(F)cc3)nc(CC(C)C)c(C(N)=O)c2-c2ccc(C(=O)N[C@@H]3CCc4ccccc43)s2)o1] |
| 186 | 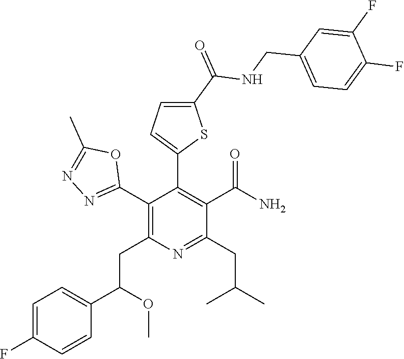 [SMILES: COC(Cc1nc(CC(C)C)c(C(N)=O)c(-c2ccc(C(=O)NCc3ccc(F)c(F)c3)s2)c1-c1nnc(C)o1)c1ccc(F)cc1] |
| 187 | 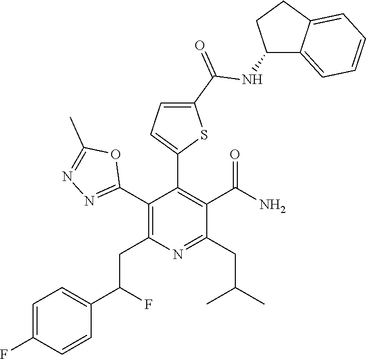 [SMILES: Cc1nnc(-c2c(CC(F)c3ccc(F)cc3)nc(CC(C)C)c(C(N)=O)c2-c2ccc(C(=O)N[C@@H]3CCc4ccccc43)s2)o1] |
| 188 | 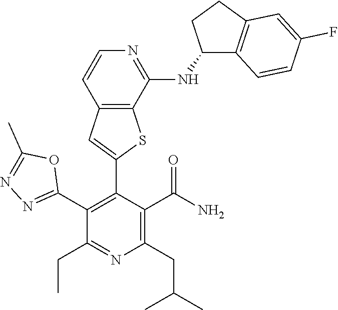 [SMILES: CCc1nc(CC(C)C)c(C(N)=O)c(-c2cc3ccnc(N[C@@H]4CCc5cc(F)ccc54)c3s2)c1-c1nnc(C)o1] |
| 189 | 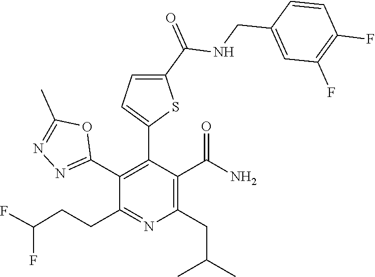 [SMILES: Cc1nnc(-c2c(CCC(F)F)nc(CC(C)C)c(C(N)=O)c2-c2ccc(C(=O)NCc3ccc(F)c(F)c3)s2)o1] |
| 190 | 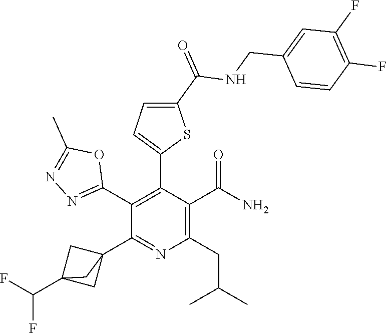 [SMILES: Cc1nnc(-c2c(C34CC(C(F)F)(C3)C4)nc(CC(C)C)c(C(N)=O)c2-c2ccc(C(=O)NCc3ccc(F)c(F)c3)s2)o1] |
| 191 | 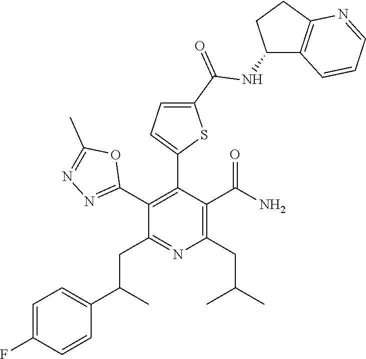 [SMILES: Cc1nnc(-c2c(CC(C)c3ccc(F)cc3)nc(CC(C)C)c(C(N)=O)c2-c2ccc(C(=O)N[C@@H]3CCc4ncccc43)s2)o1] |
| 192 | 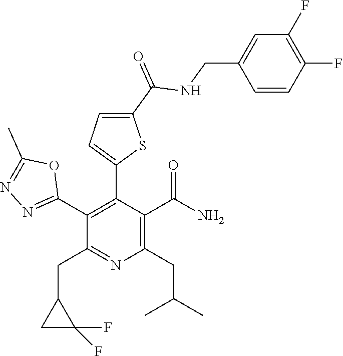 [SMILES: Cc1nnc(-c2c(CC3CC3(F)F)nc(CC(C)C)c(C(N)=O)c2-c2ccc(C(=O)NCc3ccc(F)c(F)c3)s2)o1] |
| 193 | 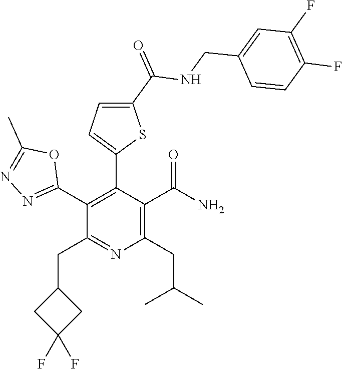 [SMILES: Cc1nnc(-c2c(CC3CC(F)(F)C3)nc(CC(C)C)c(C(N)=O)c2-c2ccc(C(=O)NCc3ccc(F)c(F)c3)s2)o1] |
| 194 | 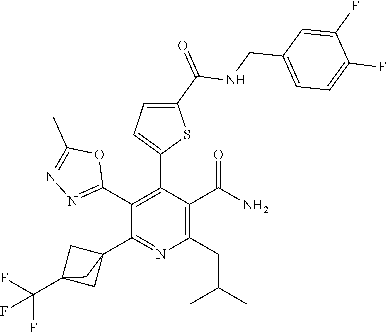 [SMILES: Cc1nnc(-c2c(C34CC(C(F)(F)F)(C3)C4)nc(CC(C)C)c(C(N)=O)c2-c2ccc(C(=O)NCc3ccc(F)c(F)c3)s2)o1] |
| 195 | 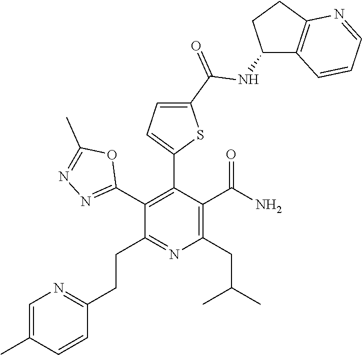 [SMILES: Cc1ccc(CCc2nc(CC(C)C)c(C(N)=O)c(-c3ccc(C(=O)N[C@@H]4CCc5ncccc54)s3)c2-c2nnc(C)o2)nc1] |
| 196 | 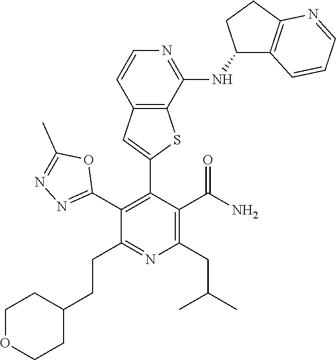 [SMILES: Cc1nnc(-c2c(CCC3CCOCC3)nc(CC(C)C)c(C(N)=O)c2-c2cc3ccnc(N[C@@H]4CCc5ncccc54)c3s2)o1] |
| 197 | 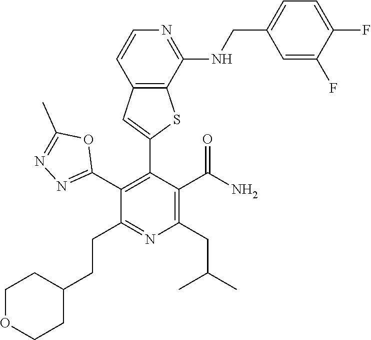 [SMILES: Cc1nnc(-c2c(CCC3CCOCC3)nc(CC(C)C)c(C(N)=O)c2-c2cc3ccnc(NCc4ccc(F)c(F)c4)c3s2)o1] |
| 198 | 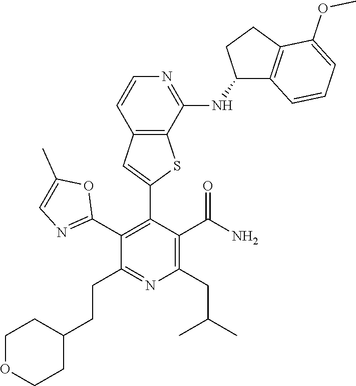 [SMILES: COc1cccc2c1CC[C@H]2Nc1nccc2cc(-c3c(C(N)=O)c(CC(C)C)nc(CCC4CCOCC4)c3-c3ncc(C)o3)sc12] |
| 199 | 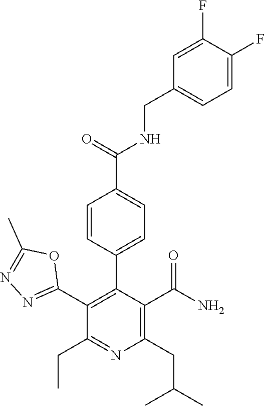 [SMILES: CCc1nc(CC(C)C)c(C(N)=O)c(-c2ccc(C(=O)NCc3ccc(F)c(F)c3)cc2)c1-c1nnc(C)o1] |
| 200 | 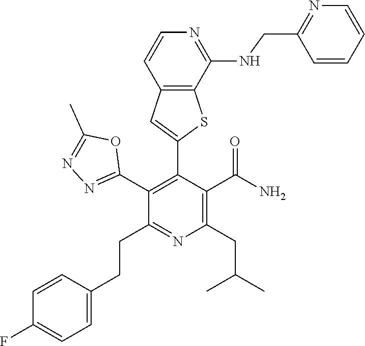 [SMILES: Cc1nnc(-c2c(CCc3ccc(F)cc3)nc(CC(C)C)c(C(N)=O)c2-c2cc3ccnc(NCc4ccccn4)c3s2)o1] |
| 201 | 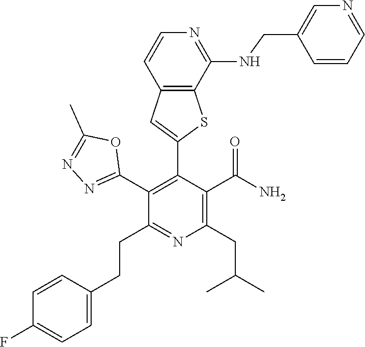 [SMILES: Cc1nnc(-c2c(CCc3ccc(F)cc3)nc(CC(C)C)c(C(N)=O)c2-c2cc3ccnc(NCc4cccnc4)c3s2)o1] |
| 202 | 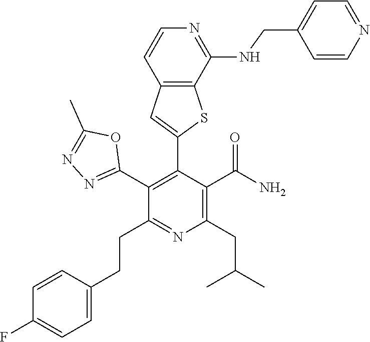 [SMILES: Cc1nnc(-c2c(CCc3ccc(F)cc3)nc(CC(C)C)c(C(N)=O)c2-c2cc3ccnc(NCc4ccncc4)c3s2)o1] |
| 203 | 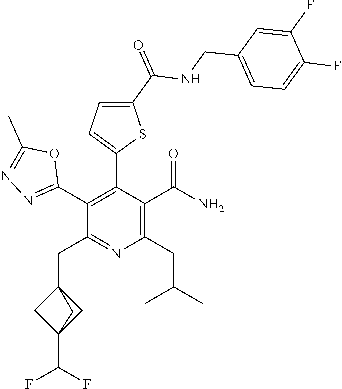 [SMILES: Cc1nnc(-c2c(CC34CC(C(F)F)(C3)C4)nc(CC(C)C)c(C(N)=O)c2-c2ccc(C(=O)NCc3ccc(F)c(F)c3)s2)o1] |
| 204 | 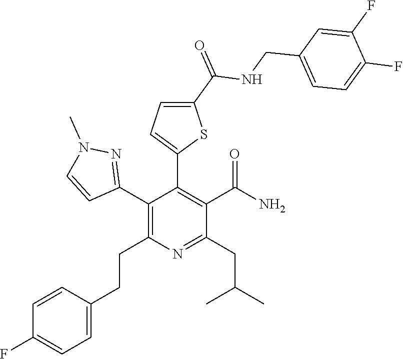 [SMILES: CC(C)Cc1nc(CCc2ccc(F)cc2)c(-c2ccn(C)n2)c(-c2ccc(C(=O)NCc3ccc(F)c(F)c3)s2)c1C(N)=O] |
| 205 | 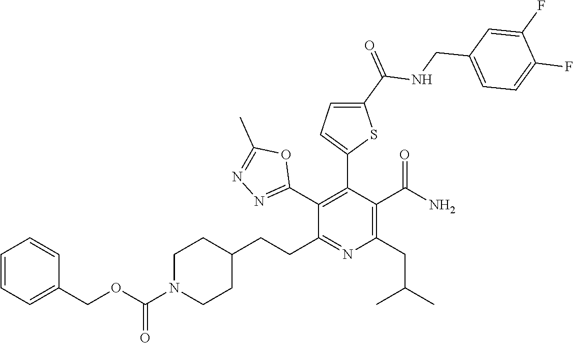 [SMILES: Cc1nnc(-c2c(CCC3CCN(C(=O)OCc4ccccc4)CC3)nc(CC(C)C)c(C(N)=O)c2-c2ccc(C(=O)NCc3ccc(F)c(F)c3)s2)o1] |
| 206 | 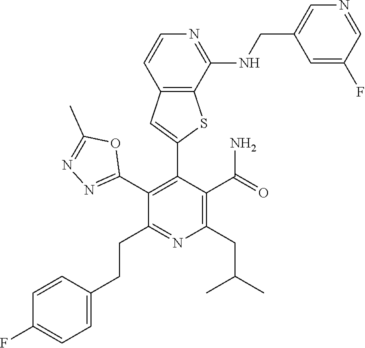 [SMILES: Cc1nnc(-c2c(CCc3ccc(F)cc3)nc(CC(C)C)c(C(N)=O)c2-c2cc3ccnc(NCc4cncc(F)c4)c3s2)o1] |
| 207 | 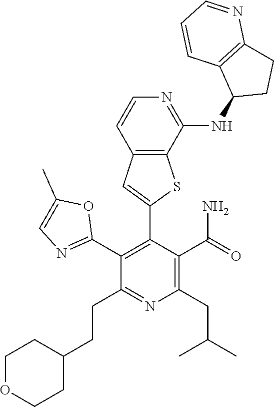 [SMILES: Cc1cnc(-c2c(CCC3CCOCC3)nc(CC(C)C)c(C(N)=O)c2-c2cc3ccnc(N[C@@H]4CCc5ncccc54)c3s2)o1] |
| 208 | 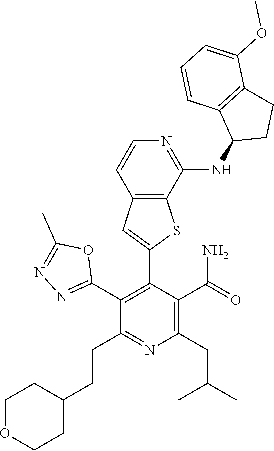 [SMILES: COc1cccc2c1CC[C@H]2Nc1nccc2cc(-c3c(C(N)=O)c(CC(C)C)nc(CCC4CCOCC4)c3-c3nnc(C)o3)sc12] |
| 209 | 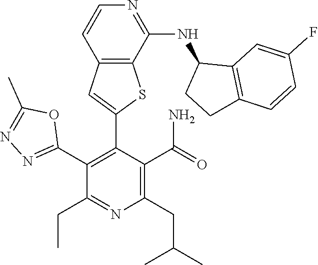 [SMILES: CCc1nc(CC(C)C)c(C(N)=O)c(-c2cc3ccnc(N[C@@H]4CCc5ccc(F)cc54)c3s2)c1-c1nnc(C)o1] |
| 210 | 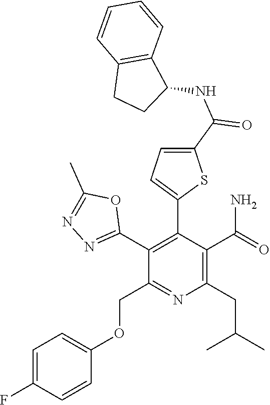 [SMILES: Cc1nnc(-c2c(COc3ccc(F)cc3)nc(CC(C)C)c(C(N)=O)c2-c2ccc(C(=O)N[C@@H]3CCc4ccccc43)s2)o1] |
| 211 | 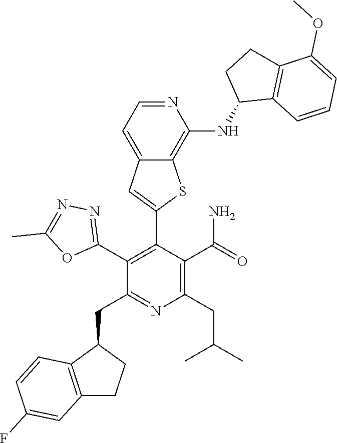 [SMILES: COc1cccc2c1CC[C@H]2Nc1nccc2cc(-c3c(C(N)=O)c(CC(C)C)nc(C[C@H]4CCc5cc(F)ccc54)c3-c3nnc(C)o3)sc12] |
| 212 | 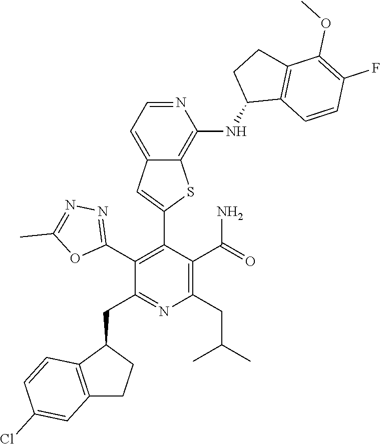 [SMILES: COc1c(F)ccc2c1CC[C@H]2Nc1nccc2cc(-c3c(C(N)=O)c(CC(C)C)nc(C[C@H]4CCc5cc(Cl)ccc54)c3-c3nnc(C)o3)sc12] |
| 213 | 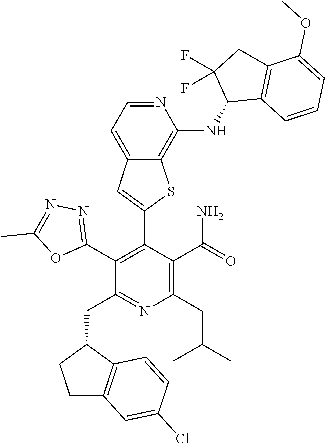 [SMILES: COc1cccc2c1CC(F)(F)[C@H]2Nc1nccc2cc(-c3c(C(N)=O)c(CC(C)C)nc(C[C@H]4CCc5cc(Cl)ccc54)c3-c3nnc(C)o3)sc12] |
| 214 | 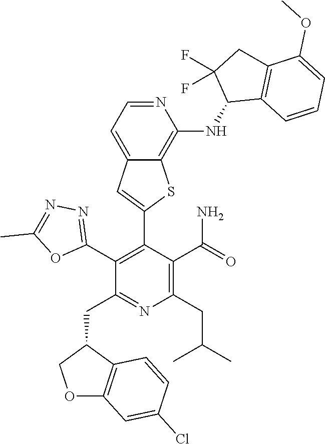 [SMILES: COc1cccc2c1CC(F)(F)[C@H]2Nc1nccc2cc(-c3c(C(N)=O)c(CC(C)C)nc(C[C@H]4COc5cc(Cl)ccc54)c3-c3nnc(C)o3)sc12] |
| 215 | 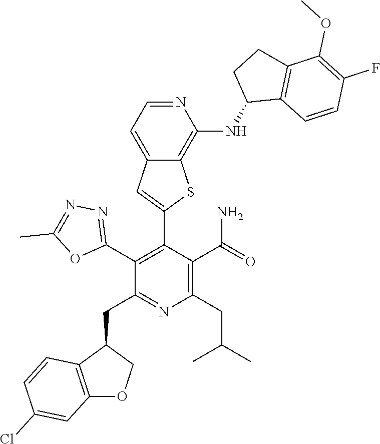 [SMILES: COc1c(F)ccc2c1CC[C@H]2Nc1nccc2cc(-c3c(C(N)=O)c(CC(C)C)nc(C[C@H]4COc5cc(Cl)ccc54)c3-c3nnc(C)o3)sc12] |

The compounds of Formula I provided herein encompass stereochemical forms of the compounds, for example, optical isomers, such as enantiomers, diastereomers, as well as mixtures thereof, e.g., mixtures of enantiomers and/or diastereomers, including racemic mixtures, as well as equal or non-equal mixtures of individual enantiomers and/or diastereomers. All stereochemical forms are contemplated in this disclosure. Unless otherwise indicated, when a disclosed compound is named or depicted by a structure without specifying the stereochemistry and has one or more chiral centers, it is understood to represent all possible stereoisomers of the compound. Representative stereochemical forms are provided throughout the specification, including but not limited to those delineated in Table 2. In some embodiments, provided is compound selected from Table 2, or a pharmaceutically acceptable salt, stereoisomer, mixture of stereoisomers, or solvate thereof:

TABLE 2
Structure

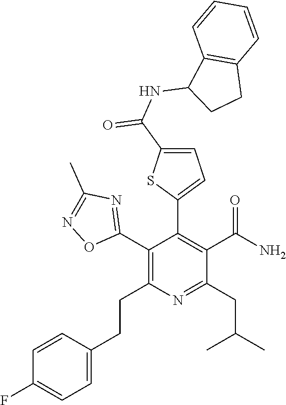 [SMILES: Cc1noc(-c2c(CCc3ccc(F)cc3)nc(CC(C)C)c(C(N)=O)c2-c2ccc(C(=O)NC3CCc4ccccc43)s2)n1]

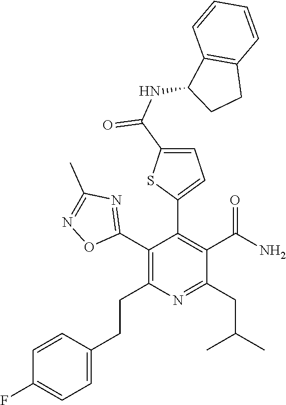 [SMILES: Cc1noc(-c2c(CCc3ccc(F)cc3)nc(CC(C)C)c(C(N)=O)c2-c2ccc(C(=O)N[C@H]3CCc4ccccc43)s2)n1]

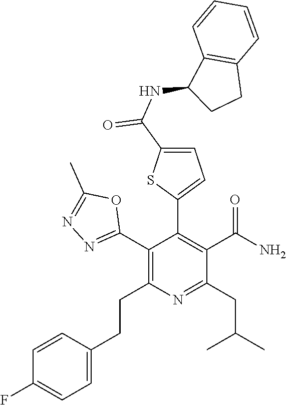 [SMILES: Cc1nnc(-c2c(CCc3ccc(F)cc3)nc(CC(C)C)c(C(N)=O)c2-c2ccc(C(=O)N[C@@H]3CCc4ccccc43)s2)o1]

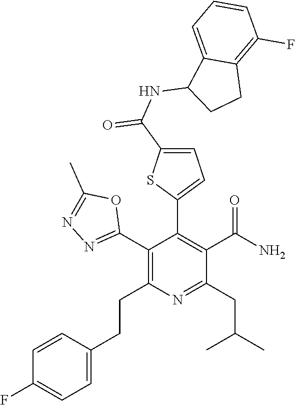 [SMILES: Cc1nnc(-c2c(CCc3ccc(F)cc3)nc(CC(C)C)c(C(N)=O)c2-c2ccc(C(=O)NC3CCc4c(F)cccc43)s2)o1]

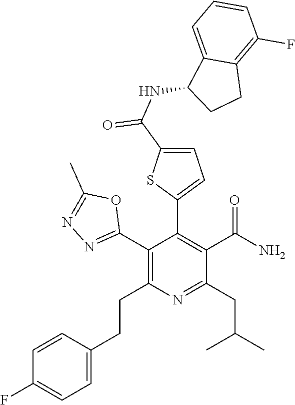 [SMILES: Cc1nnc(-c2c(CCc3ccc(F)cc3)nc(CC(C)C)c(C(N)=O)c2-c2ccc(C(=O)N[C@H]3CCc4c(F)cccc43)s2)o1]

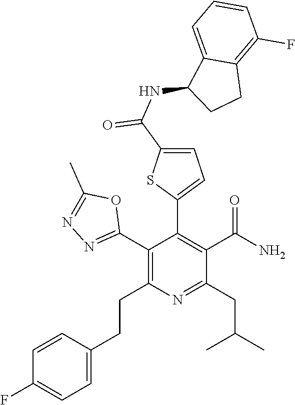 [SMILES: Cc1nnc(-c2c(CCc3ccc(F)cc3)nc(CC(C)C)c(C(N)=O)c2-c2ccc(C(=O)N[C@@H]3CCc4c(F)cccc43)s2)o1]

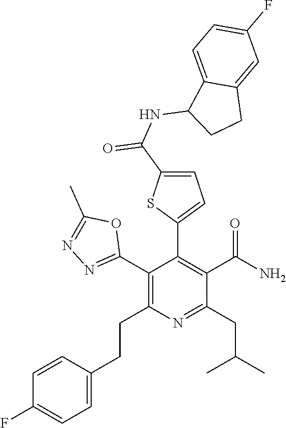 [SMILES: Cc1nnc(-c2c(CCc3ccc(F)cc3)nc(CC(C)C)c(C(N)=O)c2-c2ccc(C(=O)NC3CCc4cc(F)ccc43)s2)o1]

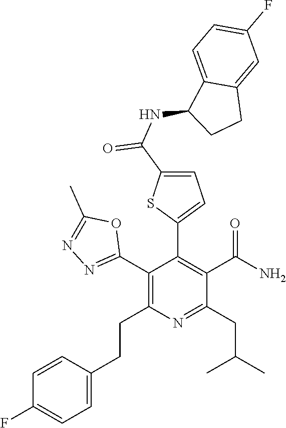 [SMILES: Cc1nnc(-c2c(CCc3ccc(F)cc3)nc(CC(C)C)c(C(N)=O)c2-c2ccc(C(=O)N[C@@H]3CCc4cc(F)ccc43)s2)o1]

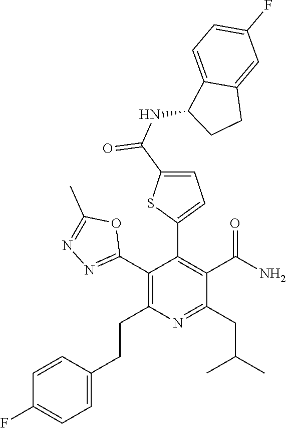 [SMILES: Cc1nnc(-c2c(CCc3ccc(F)cc3)nc(CC(C)C)c(C(N)=O)c2-c2ccc(C(=O)N[C@H]3CCc4cc(F)ccc43)s2)o1]

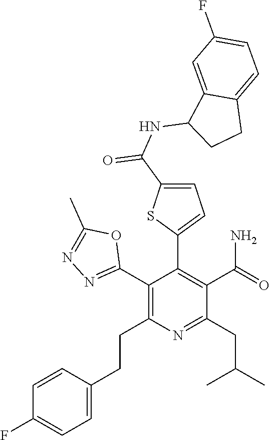 [SMILES: Cc1nnc(-c2c(CCc3ccc(F)cc3)nc(CC(C)C)c(C(N)=O)c2-c2ccc(C(=O)NC3CCc4ccc(F)cc43)s2)o1]

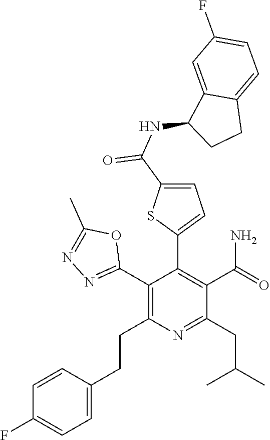 [SMILES: Cc1nnc(-c2c(CCc3ccc(F)cc3)nc(CC(C)C)c(C(N)=O)c2-c2ccc(C(=O)N[C@@H]3CCc4ccc(F)cc43)s2)o1]

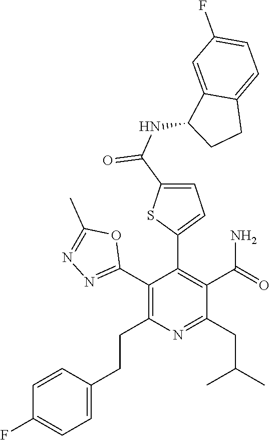 [SMILES: Cc1nnc(-c2c(CCc3ccc(F)cc3)nc(CC(C)C)c(C(N)=O)c2-c2ccc(C(=O)N[C@H]3CCc4ccc(F)cc43)s2)o1]

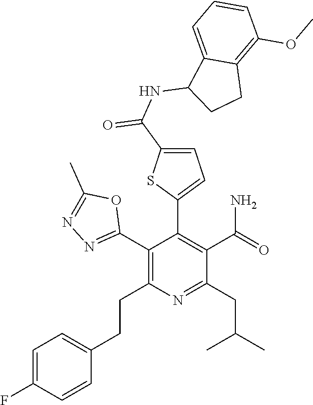 [SMILES: COc1cccc2c1CCC2NC(=O)c1ccc(-c2c(C(N)=O)c(CC(C)C)nc(CCc3ccc(F)cc3)c2-c2nnc(C)o2)s1]

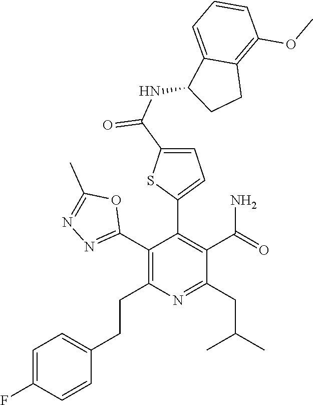 [SMILES: COc1cccc2c1CC[C@@H]2NC(=O)c1ccc(-c2c(C(N)=O)c(CC(C)C)nc(CCc3ccc(F)cc3)c2-c2nnc(C)o2)s1]

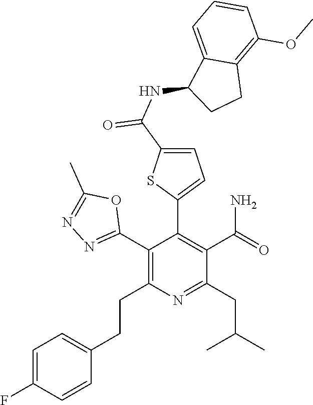 [SMILES: COc1cccc2c1CC[C@H]2NC(=O)c1ccc(-c2c(C(N)=O)c(CC(C)C)nc(CCc3ccc(F)cc3)c2-c2nnc(C)o2)s1]

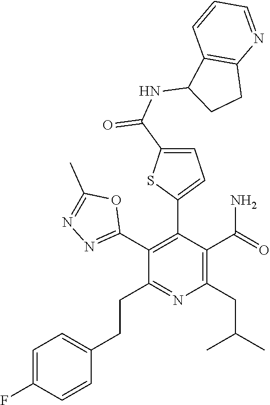 [SMILES: Cc1nnc(-c2c(CCc3ccc(F)cc3)nc(CC(C)C)c(C(N)=O)c2-c2ccc(C(=O)NC3CCc4ncccc43)s2)o1]

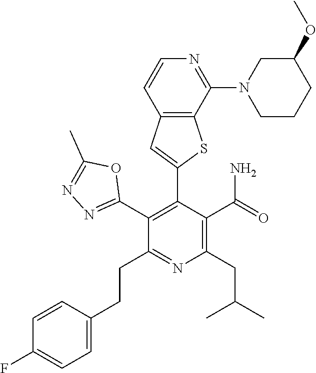 [SMILES: CO[C@H]1CCCN(c2nccc3cc(-c4c(C(N)=O)c(CC(C)C)nc(CCc5ccc(F)cc5)c4-c4nnc(C)o4)sc23)C1]

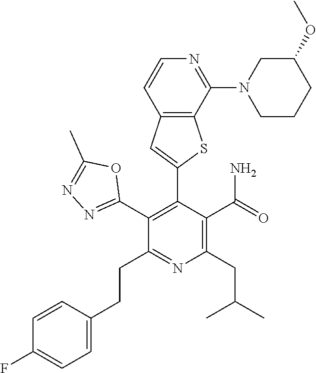 [SMILES: CO[C@@H]1CCCN(c2nccc3cc(-c4c(C(N)=O)c(CC(C)C)nc(CCc5ccc(F)cc5)c4-c4nnc(C)o4)sc23)C1]

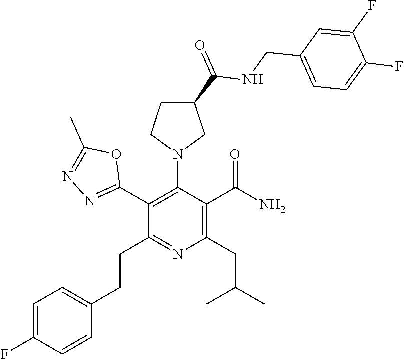 [SMILES: Cc1nnc(-c2c(CCc3ccc(F)cc3)nc(CC(C)C)c(C(N)=O)c2N2CC[C@@H](C(=O)NCc3ccc(F)c(F)c3)C2)o1]

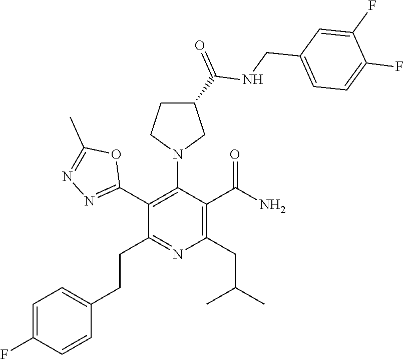 [SMILES: Cc1nnc(-c2c(CCc3ccc(F)cc3)nc(CC(C)C)c(C(N)=O)c2N2CC[C@H](C(=O)NCc3ccc(F)c(F)c3)C2)o1]

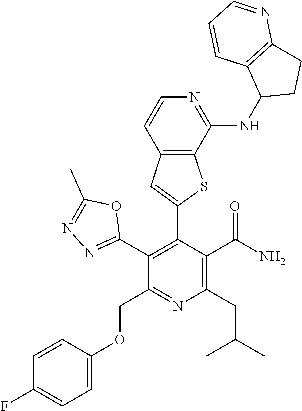 [SMILES: Cc1nnc(-c2c(COc3ccc(F)cc3)nc(CC(C)C)c(C(N)=O)c2-c2cc3ccnc(NC4CCc5ncccc54)c3s2)o1]

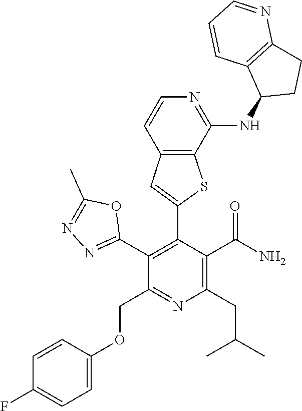 [SMILES: Cc1nnc(-c2c(COc3ccc(F)cc3)nc(CC(C)C)c(C(N)=O)c2-c2cc3ccnc(N[C@@H]4CCc5ncccc54)c3s2)o1]

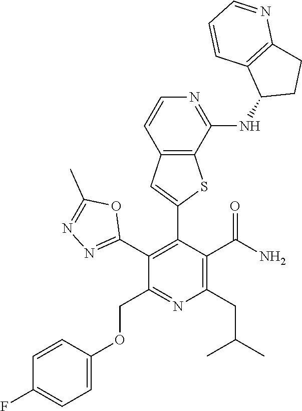 [SMILES: Cc1nnc(-c2c(COc3ccc(F)cc3)nc(CC(C)C)c(C(N)=O)c2-c2cc3ccnc(N[C@H]4CCc5ncccc54)c3s2)o1]

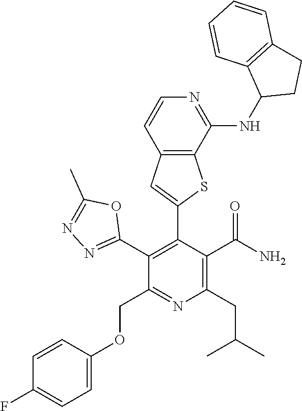 [SMILES: Cc1nnc(-c2c(COc3ccc(F)cc3)nc(CC(C)C)c(C(N)=O)c2-c2cc3ccnc(NC4CCc5ccccc54)c3s2)o1]

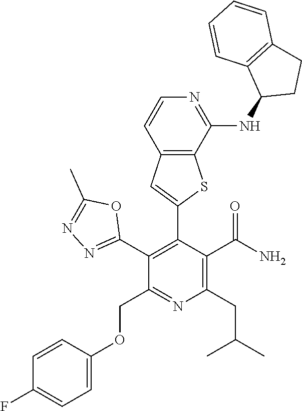 [SMILES: Cc1nnc(-c2c(COc3ccc(F)cc3)nc(CC(C)C)c(C(N)=O)c2-c2cc3ccnc(N[C@@H]4CCc5ccccc54)c3s2)o1]

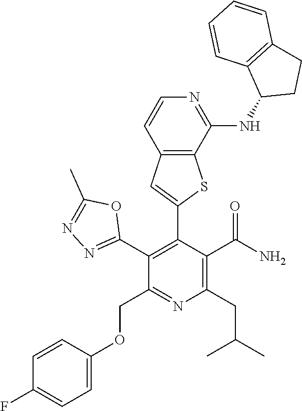 [SMILES: Cc1nnc(-c2c(COc3ccc(F)cc3)nc(CC(C)C)c(C(N)=O)c2-c2cc3ccnc(N[C@H]4CCc5ccccc54)c3s2)o1]

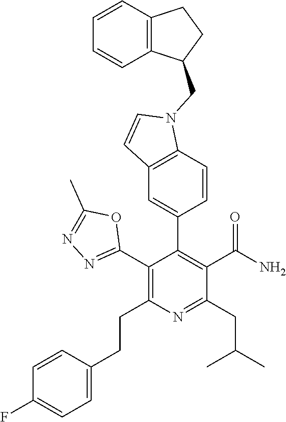 [SMILES: Cc1nnc(-c2c(CCc3ccc(F)cc3)nc(CC(C)C)c(C(N)=O)c2-c2ccc3c(ccn3C[C@@H]3CCc4ccccc43)c2)o1]

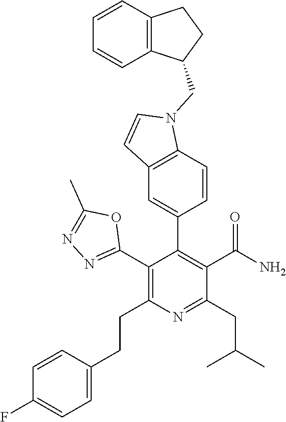 [SMILES: Cc1nnc(-c2c(CCc3ccc(F)cc3)nc(CC(C)C)c(C(N)=O)c2-c2ccc3c(ccn3C[C@H]3CCc4ccccc43)c2)o1]

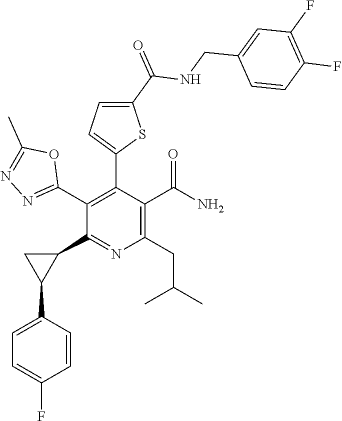 [SMILES: Cc1nnc(-c2c([C@@H]3C[C@@H]3c3ccc(F)cc3)nc(CC(C)C)c(C(N)=O)c2-c2ccc(C(=O)NCc3ccc(F)c(F)c3)s2)o1]

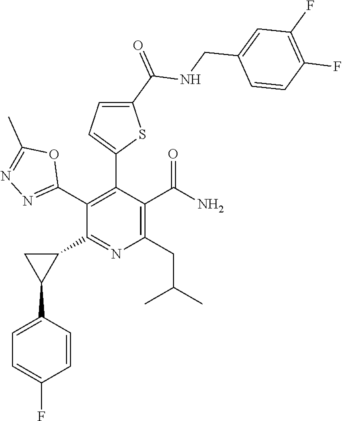 [SMILES: Cc1nnc(-c2c([C@H]3C[C@@H]3c3ccc(F)cc3)nc(CC(C)C)c(C(N)=O)c2-c2ccc(C(=O)NCc3ccc(F)c(F)c3)s2)o1]

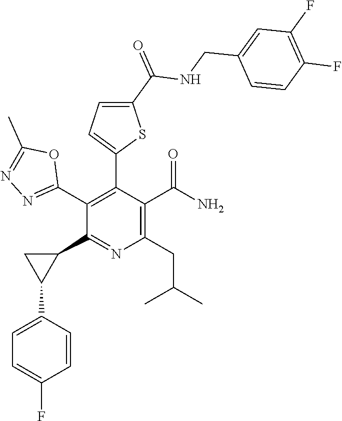 [SMILES: Cc1nnc(-c2c([C@@H]3C[C@H]3c3ccc(F)cc3)nc(CC(C)C)c(C(N)=O)c2-c2ccc(C(=O)NCc3ccc(F)c(F)c3)s2)o1]

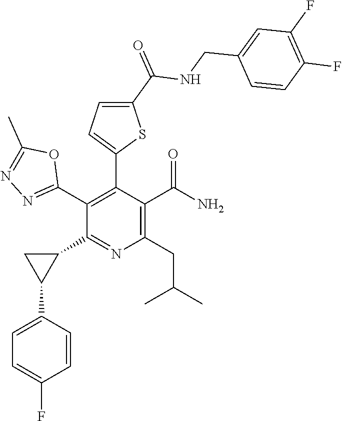 [SMILES: Cc1nnc(-c2c([C@H]3C[C@H]3c3ccc(F)cc3)nc(CC(C)C)c(C(N)=O)c2-c2ccc(C(=O)NCc3ccc(F)c(F)c3)s2)o1]

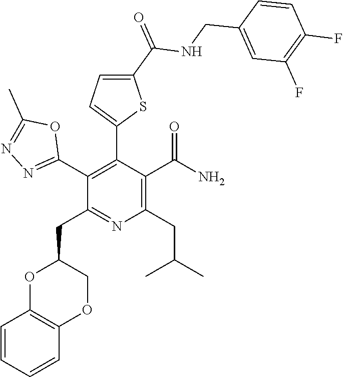 [SMILES: Cc1nnc(-c2c(C[C@H]3COc4ccccc4O3)nc(CC(C)C)c(C(N)=O)c2-c2ccc(C(=O)NCc3ccc(F)c(F)c3)s2)o1]

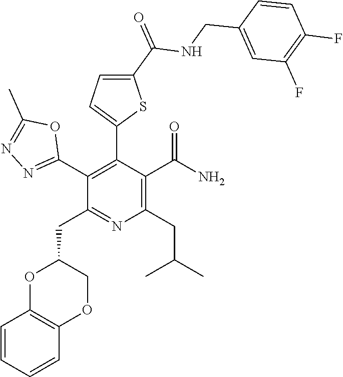 [SMILES: Cc1nnc(-c2c(C[C@@H]3COc4ccccc4O3)nc(CC(C)C)c(C(N)=O)c2-c2ccc(C(=O)NCc3ccc(F)c(F)c3)s2)o1]

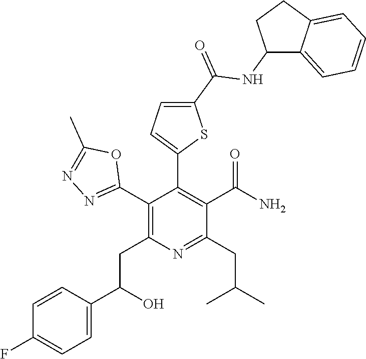 [SMILES: Cc1nnc(-c2c(CC(O)c3ccc(F)cc3)nc(CC(C)C)c(C(N)=O)c2-c2ccc(C(=O)NC3CCc4ccccc43)s2)o1]

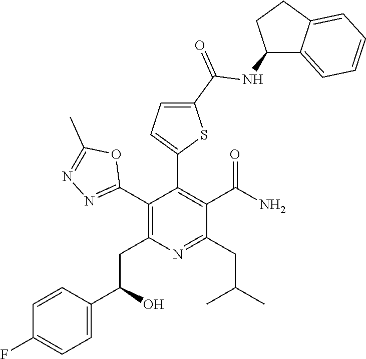 [SMILES: Cc1nnc(-c2c(C[C@@H](O)c3ccc(F)cc3)nc(CC(C)C)c(C(N)=O)c2-c2ccc(C(=O)N[C@H]3CCc4ccccc43)s2)o1]

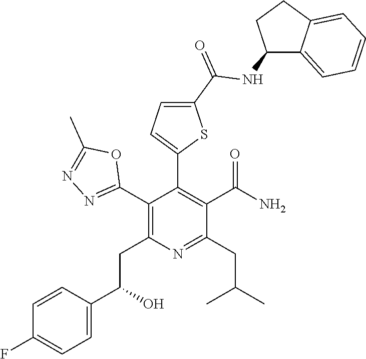 [SMILES: Cc1nnc(-c2c(C[C@H](O)c3ccc(F)cc3)nc(CC(C)C)c(C(N)=O)c2-c2ccc(C(=O)N[C@H]3CCc4ccccc43)s2)o1]

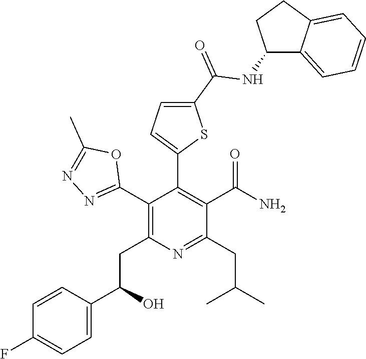 [SMILES: Cc1nnc(-c2c(C[C@@H](O)c3ccc(F)cc3)nc(CC(C)C)c(C(N)=O)c2-c2ccc(C(=O)N[C@@H]3CCc4ccccc43)s2)o1]

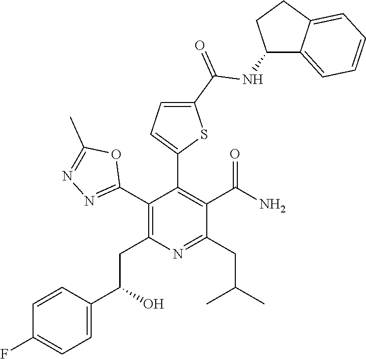 [SMILES: Cc1nnc(-c2c(C[C@H](O)c3ccc(F)cc3)nc(CC(C)C)c(C(N)=O)c2-c2ccc(C(=O)N[C@@H]3CCc4ccccc43)s2)o1]

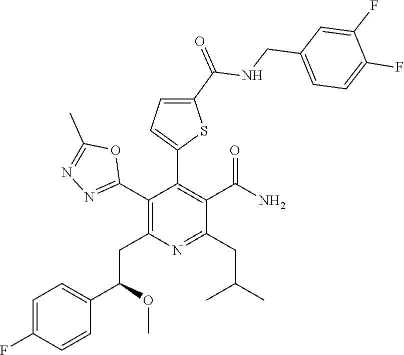 [SMILES: CO[C@H](Cc1nc(CC(C)C)c(C(N)=O)c(-c2ccc(C(=O)NCc3ccc(F)c(F)c3)s2)c1-c1nnc(C)o1)c1ccc(F)cc1]

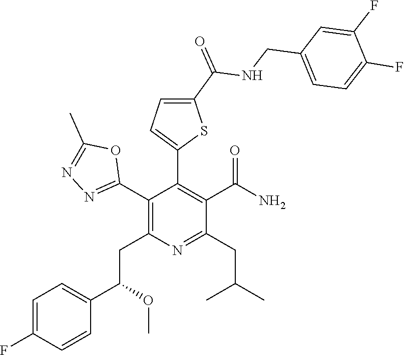 [SMILES: CO[C@@H](Cc1nc(CC(C)C)c(C(N)=O)c(-c2ccc(C(=O)NCc3ccc(F)c(F)c3)s2)c1-c1nnc(C)o1)c1ccc(F)cc1]

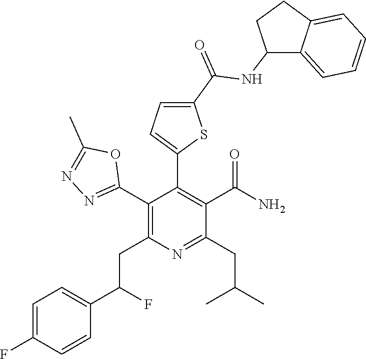 [SMILES: Cc1nnc(-c2c(CC(F)c3ccc(F)cc3)nc(CC(C)C)c(C(N)=O)c2-c2ccc(C(=O)NC3CCc4ccccc43)s2)o1]

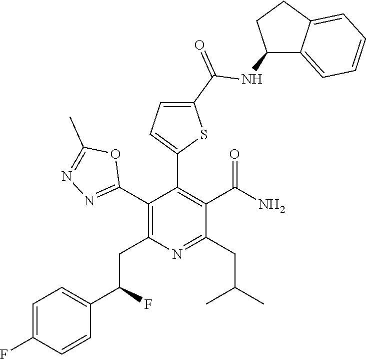 [SMILES: Cc1nnc(-c2c(C[C@@H](F)c3ccc(F)cc3)nc(CC(C)C)c(C(N)=O)c2-c2ccc(C(=O)N[C@H]3CCc4ccccc43)s2)o1]

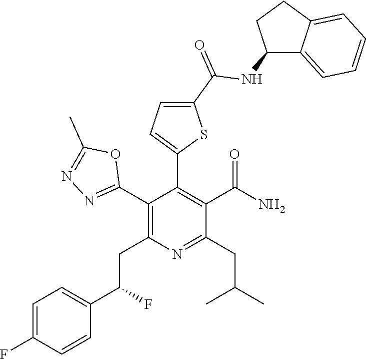 [SMILES: Cc1nnc(-c2c(C[C@H](F)c3ccc(F)cc3)nc(CC(C)C)c(C(N)=O)c2-c2ccc(C(=O)N[C@H]3CCc4ccccc43)s2)o1]

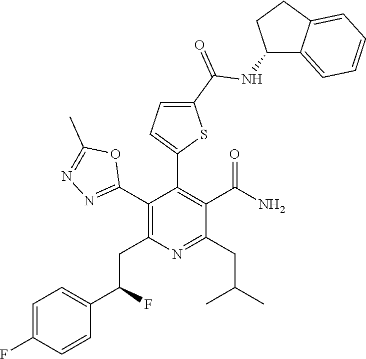 [SMILES: Cc1nnc(-c2c(C[C@@H](F)c3ccc(F)cc3)nc(CC(C)C)c(C(N)=O)c2-c2ccc(C(=O)N[C@@H]3CCc4ccccc43)s2)o1]

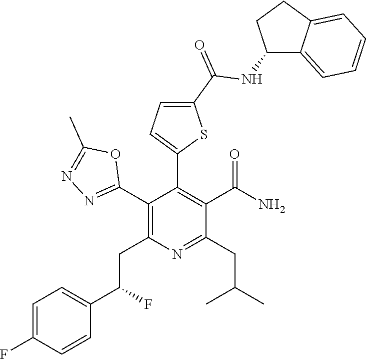 [SMILES: Cc1nnc(-c2c(C[C@H](F)c3ccc(F)cc3)nc(CC(C)C)c(C(N)=O)c2-c2ccc(C(=O)N[C@@H]3CCc4ccccc43)s2)o1]

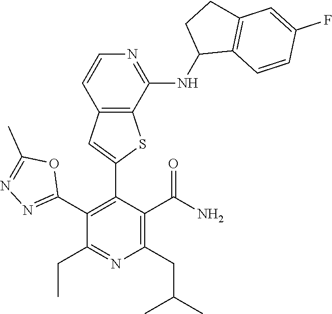 [SMILES: CCc1nc(CC(C)C)c(C(N)=O)c(-c2cc3ccnc(NC4CCc5cc(F)ccc54)c3s2)c1-c1nnc(C)o1]

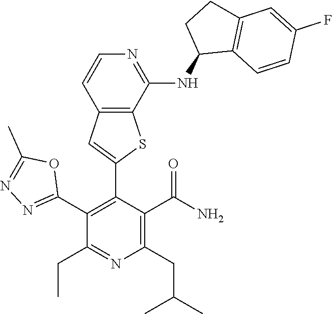 [SMILES: CCc1nc(CC(C)C)c(C(N)=O)c(-c2cc3ccnc(N[C@H]4CCc5cc(F)ccc54)c3s2)c1-c1nnc(C)o1]

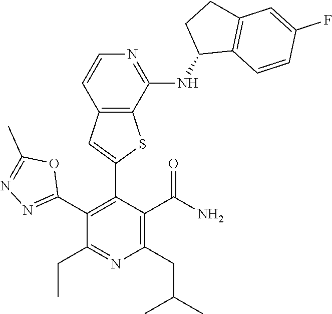 [SMILES: CCc1nc(CC(C)C)c(C(N)=O)c(-c2cc3ccnc(N[C@@H]4CCc5cc(F)ccc54)c3s2)c1-c1nnc(C)o1]

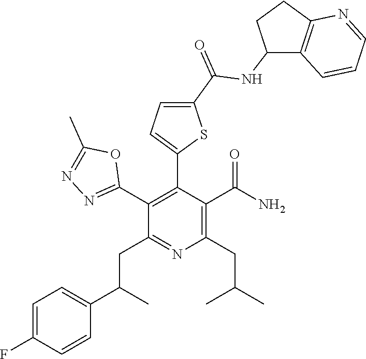 [SMILES: Cc1nnc(-c2c(CC(C)c3ccc(F)cc3)nc(CC(C)C)c(C(N)=O)c2-c2ccc(C(=O)NC3CCc4ncccc43)s2)o1]

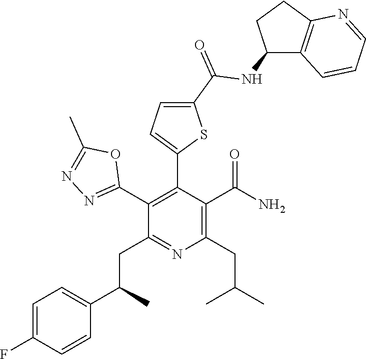 [SMILES: Cc1nnc(-c2c(C[C@@H](C)c3ccc(F)cc3)nc(CC(C)C)c(C(N)=O)c2-c2ccc(C(=O)N[C@H]3CCc4ncccc43)s2)o1]

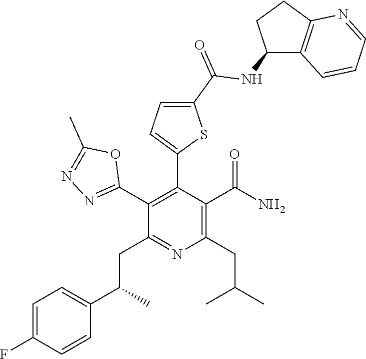 [SMILES: Cc1nnc(-c2c(C[C@H](C)c3ccc(F)cc3)nc(CC(C)C)c(C(N)=O)c2-c2ccc(C(=O)N[C@H]3CCc4ncccc43)s2)o1]

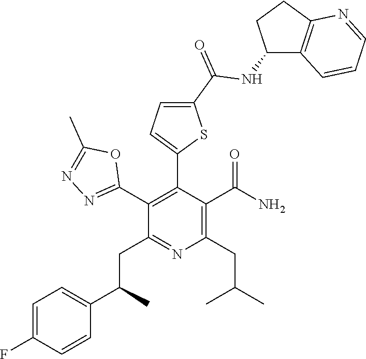 [SMILES: Cc1nnc(-c2c(C[C@@H](C)c3ccc(F)cc3)nc(CC(C)C)c(C(N)=O)c2-c2ccc(C(=O)N[C@@H]3CCc4ncccc43)s2)o1]

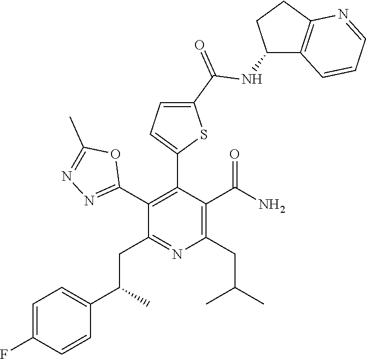 [SMILES: Cc1nnc(-c2c(C[C@H](C)c3ccc(F)cc3)nc(CC(C)C)c(C(N)=O)c2-c2ccc(C(=O)N[C@@H]3CCc4ncccc43)s2)o1]

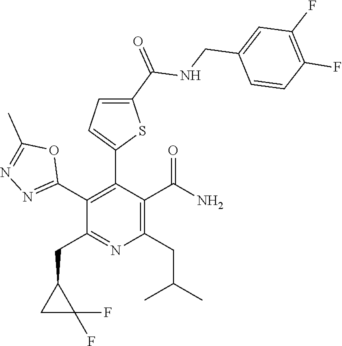 [SMILES: Cc1nnc(-c2c(C[C@@H]3CC3(F)F)nc(CC(C)C)c(C(N)=O)c2-c2ccc(C(=O)NCc3ccc(F)c(F)c3)s2)o1]

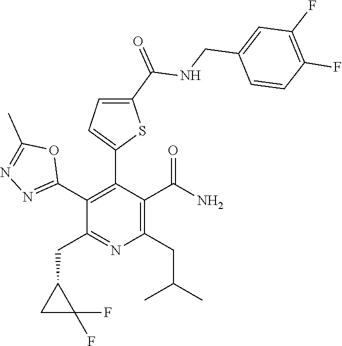 [SMILES: Cc1nnc(-c2c(C[C@H]3CC3(F)F)nc(CC(C)C)c(C(N)=O)c2-c2ccc(C(=O)NCc3ccc(F)c(F)c3)s2)o1]

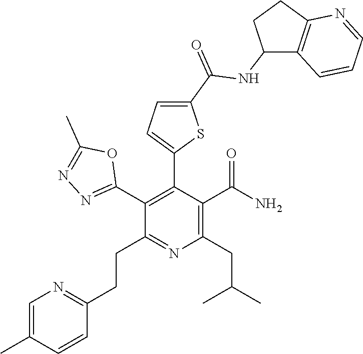 [SMILES: Cc1ccc(CCc2nc(CC(C)C)c(C(N)=O)c(-c3ccc(C(=O)NC4CCc5ncccc54)s3)c2-c2nnc(C)o2)nc1]

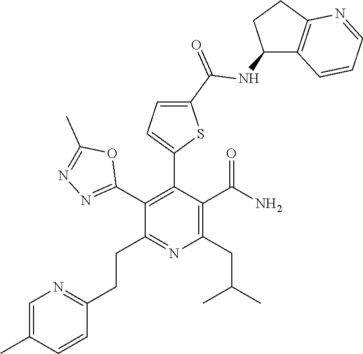 [SMILES: Cc1ccc(CCc2nc(CC(C)C)c(C(N)=O)c(-c3ccc(C(=O)N[C@H]4CCc5ncccc54)s3)c2-c2nnc(C)o2)nc1]

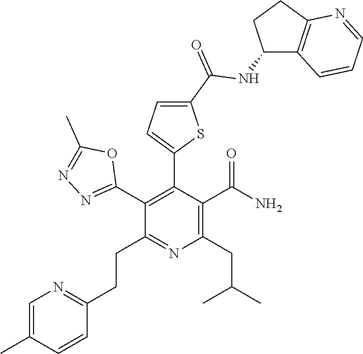 [SMILES: Cc1ccc(CCc2nc(CC(C)C)c(C(N)=O)c(-c3ccc(C(=O)N[C@@H]4CCc5ncccc54)s3)c2-c2nnc(C)o2)nc1]

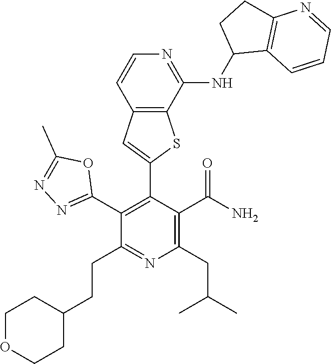 [SMILES: Cc1nnc(-c2c(CCC3CCOCC3)nc(CC(C)C)c(C(N)=O)c2-c2cc3ccnc(NC4CCc5ncccc54)c3s2)o1]

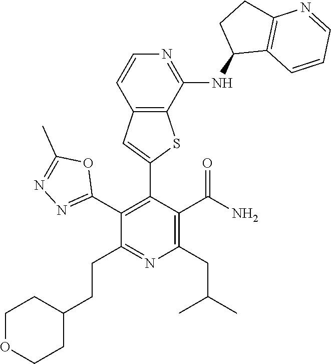 [SMILES: Cc1nnc(-c2c(CCC3CCOCC3)nc(CC(C)C)c(C(N)=O)c2-c2cc3ccnc(N[C@H]4CCc5ncccc54)c3s2)o1]

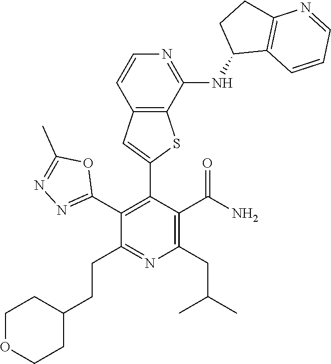 [SMILES: Cc1nnc(-c2c(CCC3CCOCC3)nc(CC(C)C)c(C(N)=O)c2-c2cc3ccnc(N[C@@H]4CCc5ncccc54)c3s2)o1]

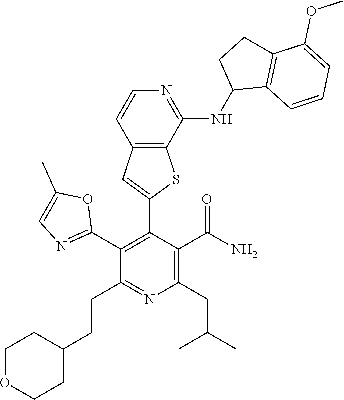 [SMILES: COc1cccc2c1CCC2Nc1nccc2cc(-c3c(C(N)=O)c(CC(C)C)nc(CCC4CCOCC4)c3-c3ncc(C)o3)sc12]

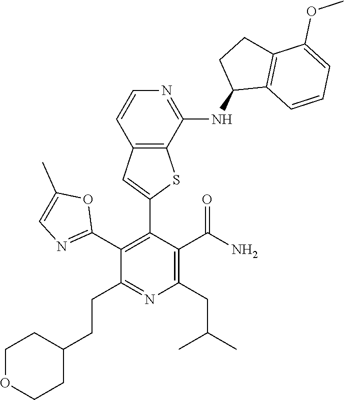 [SMILES: COc1cccc2c1CC[C@@H]2Nc1nccc2cc(-c3c(C(N)=O)c(CC(C)C)nc(CCC4CCOCC4)c3-c3ncc(C)o3)sc12]

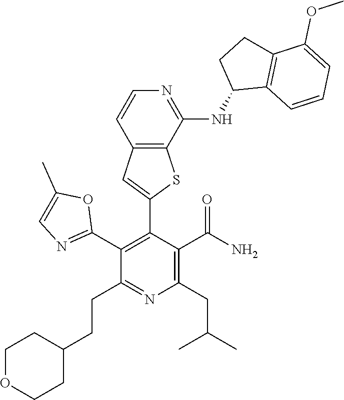 [SMILES: COc1cccc2c1CC[C@H]2Nc1nccc2cc(-c3c(C(N)=O)c(CC(C)C)nc(CCC4CCOCC4)c3-c3ncc(C)o3)sc12]

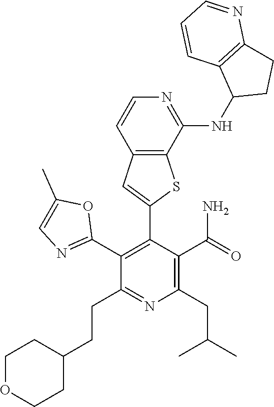 [SMILES: Cc1cnc(-c2c(CCC3CCOCC3)nc(CC(C)C)c(C(N)=O)c2-c2cc3ccnc(NC4CCc5ncccc54)c3s2)o1]

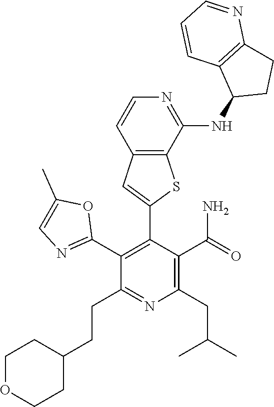 [SMILES: Cc1cnc(-c2c(CCC3CCOCC3)nc(CC(C)C)c(C(N)=O)c2-c2cc3ccnc(N[C@@H]4CCc5ncccc54)c3s2)o1]

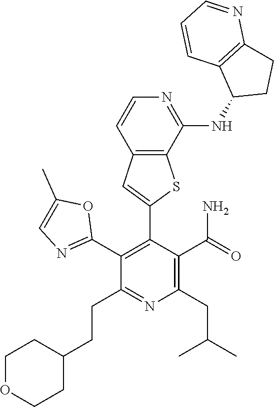 [SMILES: Cc1cnc(-c2c(CCC3CCOCC3)nc(CC(C)C)c(C(N)=O)c2-c2cc3ccnc(N[C@H]4CCc5ncccc54)c3s2)o1]

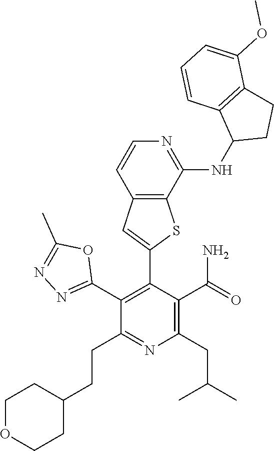 [SMILES: COc1cccc2c1CCC2Nc1nccc2cc(-c3c(C(N)=O)c(CC(C)C)nc(CCC4CCOCC4)c3-c3nnc(C)o3)sc12]

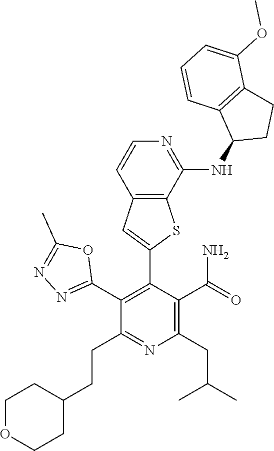 [SMILES: COc1cccc2c1CC[C@H]2Nc1nccc2cc(-c3c(C(N)=O)c(CC(C)C)nc(CCC4CCOCC4)c3-c3nnc(C)o3)sc12]

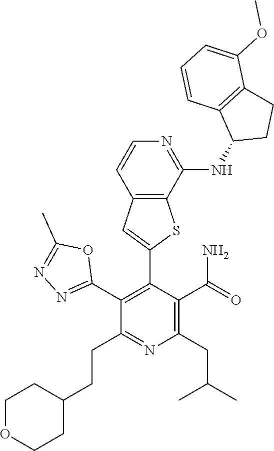 [SMILES: COc1cccc2c1CC[C@@H]2Nc1nccc2cc(-c3c(C(N)=O)c(CC(C)C)nc(CCC4CCOCC4)c3-c3nnc(C)o3)sc12]

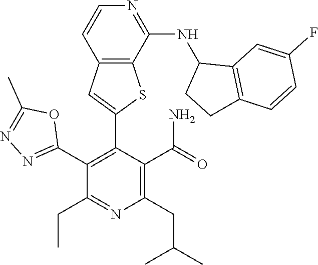 [SMILES: CCc1nc(CC(C)C)c(C(N)=O)c(-c2cc3ccnc(NC4CCc5ccc(F)cc54)c3s2)c1-c1nnc(C)o1]

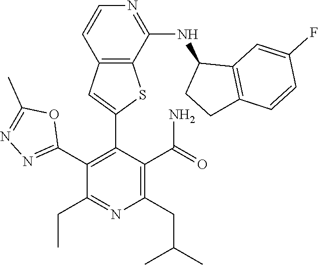 [SMILES: CCc1nc(CC(C)C)c(C(N)=O)c(-c2cc3ccnc(N[C@@H]4CCc5ccc(F)cc54)c3s2)c1-c1nnc(C)o1]

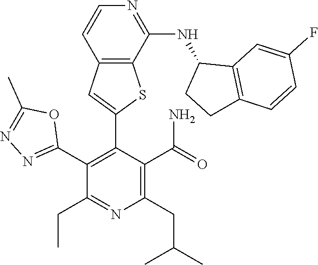 [SMILES: CCc1nc(CC(C)C)c(C(N)=O)c(-c2cc3ccnc(N[C@H]4CCc5ccc(F)cc54)c3s2)c1-c1nnc(C)o1]

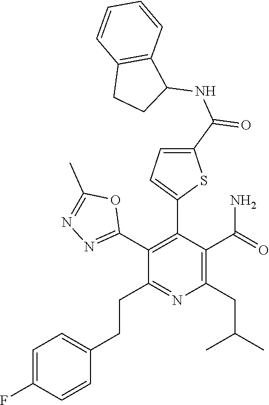 [SMILES: Cc1nnc(-c2c(CCc3ccc(F)cc3)nc(CC(C)C)c(C(N)=O)c2-c2ccc(C(=O)NC3CCc4ccccc43)s2)o1]

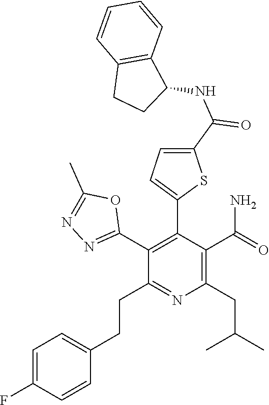 [SMILES: Cc1nnc(-c2c(CCc3ccc(F)cc3)nc(CC(C)C)c(C(N)=O)c2-c2ccc(C(=O)N[C@@H]3CCc4ccccc43)s2)o1]

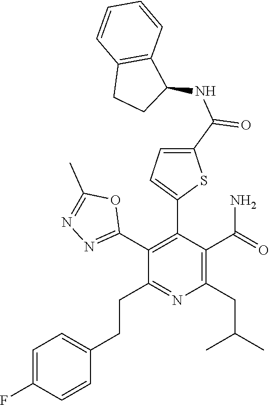 [SMILES: Cc1nnc(-c2c(CCc3ccc(F)cc3)nc(CC(C)C)c(C(N)=O)c2-c2ccc(C(=O)N[C@H]3CCc4ccccc43)s2)o1]

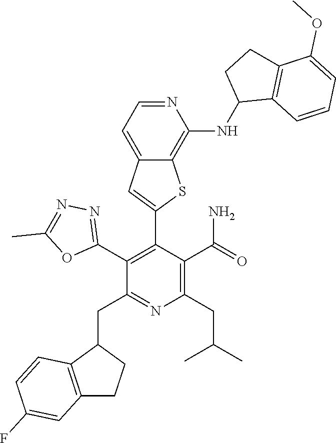 [SMILES: COc1cccc2c1CCC2Nc1nccc2cc(-c3c(C(N)=O)c(CC(C)C)nc(CC4CCc5cc(F)ccc54)c3-c3nnc(C)o3)sc12]

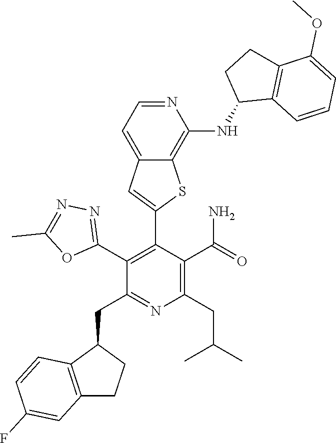 [SMILES: COc1cccc2c1CC[C@H]2Nc1nccc2cc(-c3c(C(N)=O)c(CC(C)C)nc(C[C@H]4CCc5cc(F)ccc54)c3-c3nnc(C)o3)sc12]

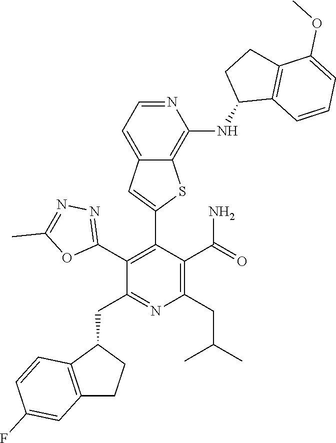 [SMILES: COc1cccc2c1CC[C@H]2Nc1nccc2cc(-c3c(C(N)=O)c(CC(C)C)nc(C[C@@H]4CCc5cc(F)ccc54)c3-c3nnc(C)o3)sc12]

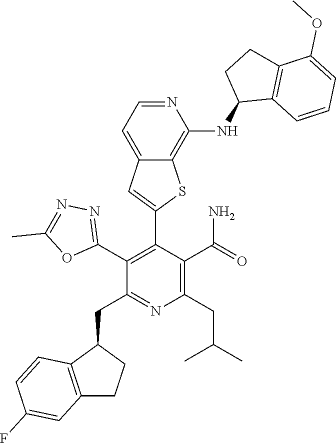 [SMILES: COc1cccc2c1CC[C@@H]2Nc1nccc2cc(-c3c(C(N)=O)c(CC(C)C)nc(C[C@H]4CCc5cc(F)ccc54)c3-c3nnc(C)o3)sc12]

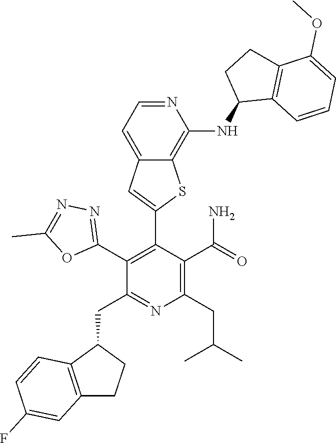 [SMILES: COc1cccc2c1CC[C@@H]2Nc1nccc2cc(-c3c(C(N)=O)c(CC(C)C)nc(C[C@@H]4CCc5cc(F)ccc54)c3-c3nnc(C)o3)sc12]

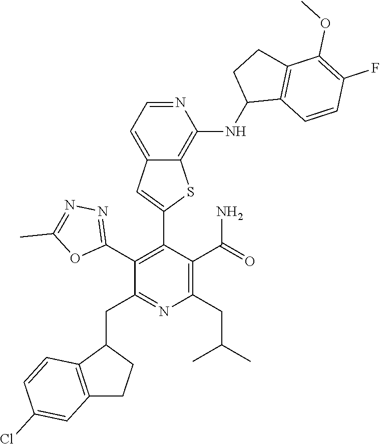 [SMILES: COc1c(F)ccc2c1CCC2Nc1nccc2cc(-c3c(C(N)=O)c(CC(C)C)nc(CC4CCc5cc(Cl)ccc54)c3-c3nnc(C)o3)sc12]

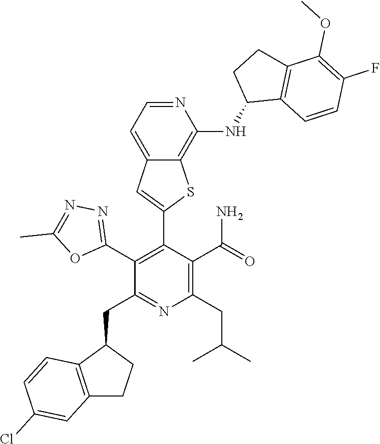 [SMILES: COc1c(F)ccc2c1CC[C@H]2Nc1nccc2cc(-c3c(C(N)=O)c(CC(C)C)nc(C[C@H]4CCc5cc(Cl)ccc54)c3-c3nnc(C)o3)sc12]

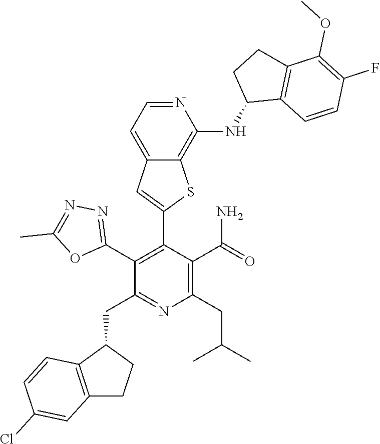 [SMILES: COc1c(F)ccc2c1CC[C@H]2Nc1nccc2cc(-c3c(C(N)=O)c(CC(C)C)nc(C[C@@H]4CCc5cc(Cl)ccc54)c3-c3nnc(C)o3)sc12]

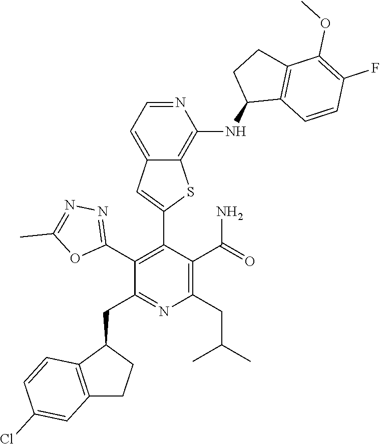 [SMILES: COc1c(F)ccc2c1CC[C@@H]2Nc1nccc2cc(-c3c(C(N)=O)c(CC(C)C)nc(C[C@H]4CCc5cc(Cl)ccc54)c3-c3nnc(C)o3)sc12]

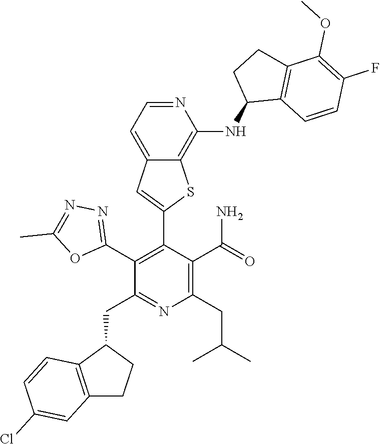 [SMILES: COc1c(F)ccc2c1CC[C@@H]2Nc1nccc2cc(-c3c(C(N)=O)c(CC(C)C)nc(C[C@@H]4CCc5cc(Cl)ccc54)c3-c3nnc(C)o3)sc12]

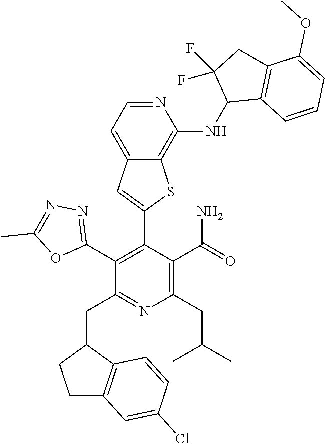 [SMILES: COc1cccc2c1CC(F)(F)C2Nc1nccc2cc(-c3c(C(N)=O)c(CC(C)C)nc(CC4CCc5cc(Cl)ccc54)c3-c3nnc(C)o3)sc12]

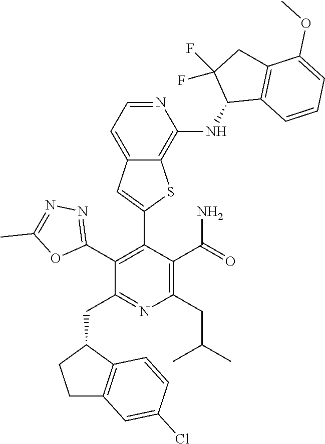 [SMILES: COc1cccc2c1CC(F)(F)[C@H]2Nc1nccc2cc(-c3c(C(N)=O)c(CC(C)C)nc(C[C@H]4CCc5cc(Cl)ccc54)c3-c3nnc(C)o3)sc12]

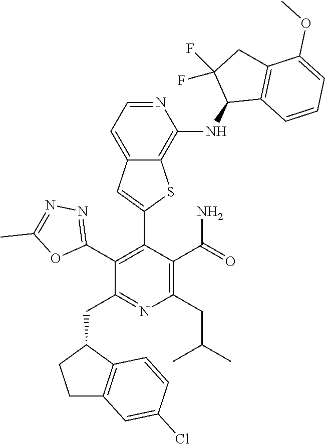 [SMILES: COc1cccc2c1CC(F)(F)[C@@H]2Nc1nccc2cc(-c3c(C(N)=O)c(CC(C)C)nc(C[C@H]4CCc5cc(Cl)ccc54)c3-c3nnc(C)o3)sc12]

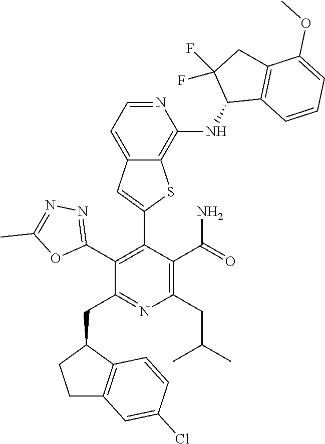 [SMILES: COc1cccc2c1CC(F)(F)[C@H]2Nc1nccc2cc(-c3c(C(N)=O)c(CC(C)C)nc(C[C@@H]4CCc5cc(Cl)ccc54)c3-c3nnc(C)o3)sc12]

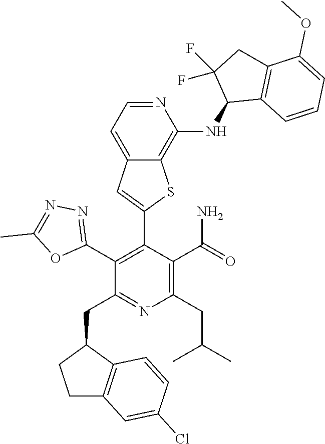 [SMILES: COc1cccc2c1CC(F)(F)[C@@H]2Nc1nccc2cc(-c3c(C(N)=O)c(CC(C)C)nc(C[C@@H]4CCc5cc(Cl)ccc54)c3-c3nnc(C)o3)sc12]

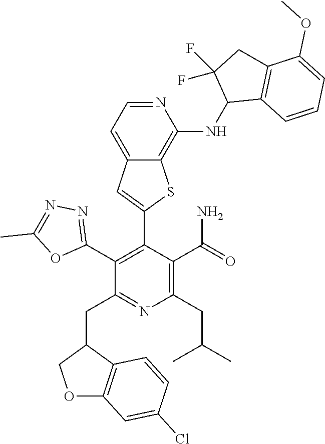 [SMILES: COc1cccc2c1CC(F)(F)C2Nc1nccc2cc(-c3c(C(N)=O)c(CC(C)C)nc(CC4COc5cc(Cl)ccc54)c3-c3nnc(C)o3)sc12]

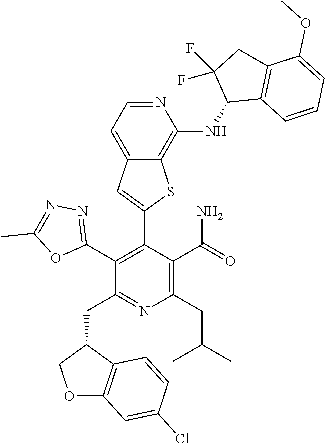 [SMILES: COc1cccc2c1CC(F)(F)[C@H]2Nc1nccc2cc(-c3c(C(N)=O)c(CC(C)C)nc(C[C@H]4COc5cc(Cl)ccc54)c3-c3nnc(C)o3)sc12]

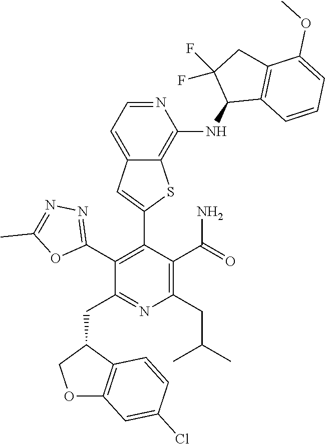 [SMILES: COc1cccc2c1CC(F)(F)[C@@H]2Nc1nccc2cc(-c3c(C(N)=O)c(CC(C)C)nc(C[C@H]4COc5cc(Cl)ccc54)c3-c3nnc(C)o3)sc12]

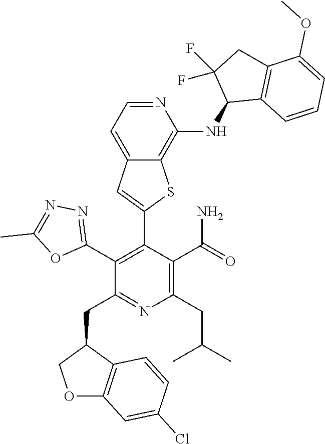 [SMILES: COc1cccc2c1CC(F)(F)[C@@H]2Nc1nccc2cc(-c3c(C(N)=O)c(CC(C)C)nc(C[C@@H]4COc5cc(Cl)ccc54)c3-c3nnc(C)o3)sc12]

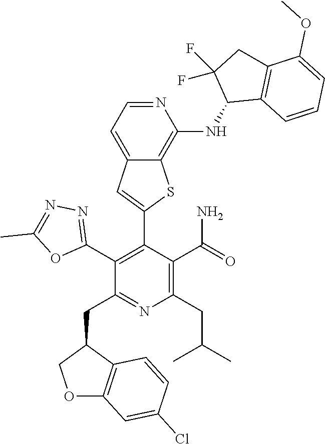 [SMILES: COc1cccc2c1CC(F)(F)[C@H]2Nc1nccc2cc(-c3c(C(N)=O)c(CC(C)C)nc(C[C@@H]4COc5cc(Cl)ccc54)c3-c3nnc(C)o3)sc12]

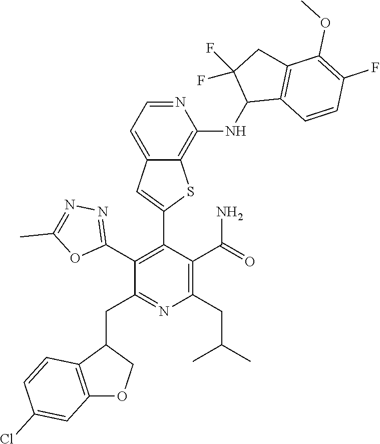 [SMILES: COc1c(F)ccc2c1CC(F)(F)C2Nc1nccc2cc(-c3c(C(N)=O)c(CC(C)C)nc(CC4COc5cc(Cl)ccc54)c3-c3nnc(C)o3)sc12]

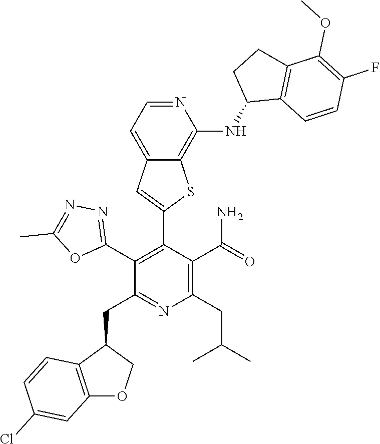 [SMILES: COc1c(F)ccc2c1CC[C@H]2Nc1nccc2cc(-c3c(C(N)=O)c(CC(C)C)nc(C[C@H]4COc5cc(Cl)ccc54)c3-c3nnc(C)o3)sc12]

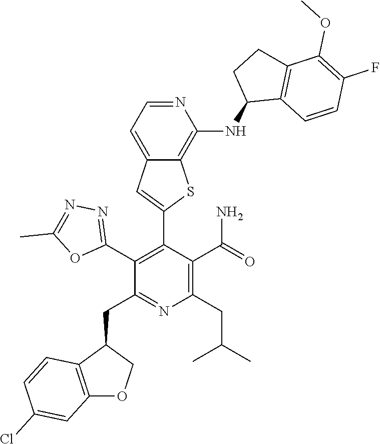 [SMILES: COc1c(F)ccc2c1CC[C@@H]2Nc1nccc2cc(-c3c(C(N)=O)c(CC(C)C)nc(C[C@H]4COc5cc(Cl)ccc54)c3-c3nnc(C)o3)sc12]

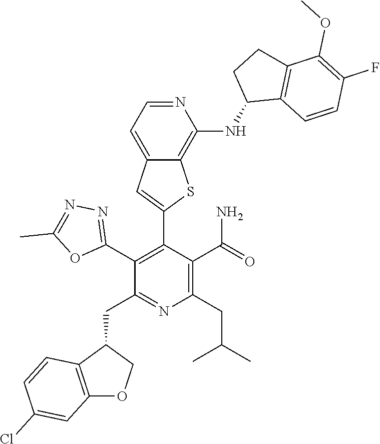 [SMILES: COc1c(F)ccc2c1CC[C@H]2Nc1nccc2cc(-c3c(C(N)=O)c(CC(C)C)nc(C[C@@H]4COc5cc(Cl)ccc54)c3-c3nnc(C)o3)sc12]

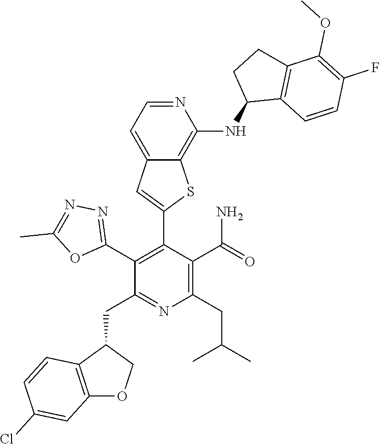 [SMILES: COc1c(F)ccc2c1CC[C@@H]2Nc1nccc2cc(-c3c(C(N)=O)c(CC(C)C)nc(C[C@@H]4COc5cc(Cl)ccc54)c3-c3nnc(C)o3)sc12]

The compounds of Formula I and subformulas thereof include pharmaceutically acceptable salts thereof. In addition, the compounds of Formula I and subformulas thereof also include other salts of such compounds which are not necessarily pharmaceutically acceptable salts, and which may be useful as intermediates for preparing and/or purifying compounds of Formula I and subformulas thereof and/or for separating enantiomers of compounds of Formula I and subformulas thereof.

It will further be appreciated that the compounds of Formula I and subformulas or their salts may be isolated in the form of solvates, and accordingly that any such solvate is included within the scope of the present disclosure. For example, compounds of Formula I and subformulas thereof and salts of each thereof can exist in unsolvated as well as solvated forms with pharmaceutically acceptable solvents such as water, ethanol, and the like.

### Pharmaceutical Compositions and Administration

When employed as pharmaceuticals, compounds as described herein (e.g., one or more compounds as disclosed herein, or a stereoisomer or mixture of stereoisomers thereof) can be administered in the form of a pharmaceutical compositions. These compositions can be prepared in a manner well known in the pharmaceutical art, and can be administered by a variety of routes, depending upon whether local or systemic treatment is desired and upon the area to be treated. Administration can be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Oral administration can include a dosage form formulated for once-daily or twice-daily (BID) administration. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or can be, for example, by a continuous perfusion pump. Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like may be necessary or desirable.

Also provided herein are pharmaceutical compositions which contain, as the active ingredient, one or more compounds as disclosed herein, or a stereoisomer or mixture of stereoisomers thereof, in combination with one or more pharmaceutically acceptable excipients (carriers). For example, a pharmaceutical composition prepared using one or more compounds as disclosed herein, or a stereoisomer or mixture of stereoisomers thereof.

In one embodiment, provided is a pharmaceutical composition comprising a compound, or a stereoisomer or mixture of stereoisomers thereof, or pharmaceutically acceptable salt thereof, as disclosed herein, and a pharmaceutically acceptable excipient. In one embodiment, provided is a pharmaceutical composition comprising a compound, or a stereoisomer or mixture of stereoisomers thereof, or pharmaceutically acceptable salt thereof, as disclosed herein, and a pharmaceutically acceptable excipient, wherein a compound, or a stereoisomer or mixture of stereoisomers thereof, or pharmaceutically acceptable salt thereof, is present in the pharmaceutical composition in an amount greater than about 0.1%, greater than about 1%, greater than about 5%, greater than about 10%, greater than about 15%, greater than about 20%, greater than about 25%, greater than about 35%, or greater than about 40%, or greater than about 45%, or greater than about 50%, or greater than about 55%, or greater than about 60%, or greater than about 65%, or greater than about 70%, or greater than about 75%, or greater than about 80%, or greater than about 85%, or greater than about 90%, or greater than about 95% purity, or about 40%, or about 45%, or about 50%, or about 55%, or about 60%, or about 65%, or about 70%, or about 75%, or about 80%, or about 85%, or about 90%, or about 95%, by weight.

In some embodiments, the composition is suitable for topical administration. In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In some embodiments, the composition is formulated for oral administration. In some embodiments, the composition is a solid oral formulation. In some embodiments, the composition is formulated as a tablet or capsule.

Further provided herein are pharmaceutical compositions containing one or more compounds as disclosed herein, or a stereoisomer or mixture of stereoisomers thereof with a pharmaceutically acceptable excipient. Pharmaceutical compositions containing one or more compounds as disclosed herein, or a stereoisomer or mixture of stereoisomers thereof as the active ingredient can be prepared by intimately mixing one or more compounds as disclosed herein, or a stereoisomer or mixture of stereoisomers thereof with a pharmaceutical carrier according to conventional pharmaceutical compounding techniques. The carrier can take a wide variety of forms depending upon the desired route of administration (e.g., oral, parenteral). In some embodiments, the composition is a solid oral composition.

Suitable pharmaceutically acceptable carriers are well known in the art. Descriptions of some of these pharmaceutically acceptable carriers can be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

In some embodiments, the compound or pharmaceutical composition can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22nd Edition (Pharmaceutical Press, London, UK. 2012).

In some embodiments, the compounds and pharmaceutical compositions described herein or a pharmaceutical composition thereof can be administered to patient in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal (e.g., intranasal), nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In some embodiments, a route of administration is parenteral (e.g., intratumoral).

In some embodiments, one or more compounds as disclosed herein, or a stereoisomer or mixture of stereoisomers thereof as described herein or pharmaceutical compositions thereof can be formulated for parenteral administration, e.g., formulated for injection via the intraarterial, intrasternal, intracranial, intravenous, intramuscular, sub-cutaneous, or intraperitoneal routes. For example, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure. In some embodiments, devices are used for parenteral administration. For example, such devices may include needle injectors, microneedle injectors, needle-free injectors, and infusion techniques.

In some embodiments, the pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In some embodiments, the form must be sterile and must be fluid to the extent that it may be easily injected. In some embodiments, the form should be stable under the conditions of manufacture and storage and must be preserved against the contaminating action of microorganisms, such as bacteria and fungi.

In some embodiments, the carrier also can be a solvent or dispersion medium containing, for example, water, ethanol, polyol (for example, glycerol, propylene glycol, and liquid polyethylene glycol, and the like), suitable mixtures thereof, and vegetable oils. In some embodiments, the proper fluidity can be maintained, for example, by the use of a coating, such as lecithin, by the maintenance of the required particle size in the case of dispersion, and by the use of surfactants. In some embodiments, the prevention of the action of microorganisms can be brought about by various antibacterial and antifungal agents, for example, parabens, chlorobutanol, phenol, sorbic acid, thimerosal, and the like. In some embodiments, isotonic agents, for example, sugars or sodium chloride are included. In some embodiments, prolonged absorption of the injectable compositions can be brought about by the use in the compositions of agents delaying absorption, for example, aluminum monostearate and gelatin.

In some embodiments, sterile injectable solutions are prepared by incorporating one or more compounds as disclosed herein, or a stereoisomer or mixture of stereoisomers thereof in the required amount in the appropriate solvent with various of the other ingredients enumerated above, as required, followed by filtered sterilization. In some embodiments, dispersions are prepared by incorporating the various sterilized active ingredients into a sterile vehicle which contains the basic dispersion medium and the required other ingredients from those enumerated above. In some embodiments, sterile powders are used for the preparation of sterile injectable solutions. In some embodiments, the methods of preparation are vacuum-drying and freeze-drying techniques, which yield a powder of the active ingredient, plus any additional desired ingredient from a previously sterile-filtered solution thereof.

In some embodiments, pharmacologically acceptable excipients usable in a rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol, Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In some embodiments, suppositories can be prepared by mixing one or more compounds as disclosed herein, or a stereoisomer or mixture of stereoisomers thereof, or pharmaceutical compositions as described herein with suitable non-irritating excipients or carriers such as cocoa butter, polyethylene glycol or a suppository wax which are solid at ambient temperature but liquid at body temperature and therefore melt in the rectum and release the active compound. In some embodiments, compositions for rectal administration are in the form of an enema.

In some embodiments, one or more compounds as disclosed herein, or a stereoisomer or mixture of stereoisomers thereof, as described herein or a pharmaceutical composition thereof is formulated for local delivery to the digestive or GI tract by way of oral administration (e.g., solid or liquid dosage forms).

In some embodiments, solid dosage forms for oral administration include capsules, tablets, pills, powders, and granules. In some embodiments, one or more compounds as disclosed herein, or a stereoisomer or mixture of stereoisomers thereof, is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For example, in the case of capsules, tablets and pills, the dosage form may also comprise buffering agents. In some embodiments, solid compositions of a similar type may also be employed as fillers in soft and hard-filled gelatin capsules using such excipients as lactose or milk sugar as well as high molecular weight polyethylene glycols and the like.

In some embodiments, the pharmaceutical compositions will take the form of a unit dosage form such as a pill or tablet and thus the composition may contain, along with one or more compounds as disclosed herein, or a stereoisomer or mixture of stereoisomers thereof as provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In some embodiments, another solid dosage form, a powder, marume, solution or suspension (e.g., in propylene carbonate, vegetable oils, PEG's, poloxamer 124 or triglycerides) is encapsulated in a capsule (gelatin or cellulose base capsule). In some embodiments, unit dosage forms in which one or more compounds and pharmaceutical compositions as provided herein or additional active agents are physically separated are also contemplated; e.g., capsules with granules (or tablets in a capsule) of each drug; two-layer tablets; two-compartment gel caps, etc. In some embodiments, enteric coated or delayed release oral dosage forms are also contemplated.

In some embodiments, other physiologically acceptable compounds may include wetting agents, emulsifying agents, dispersing agents or preservatives that are particularly useful for preventing the growth or action of microorganisms. For example, various preservatives are well known and include, for example, phenol and ascorbic acid.

In some embodiments, the excipients are sterile and generally free of undesirable matter. For example, these compositions can be sterilized by conventional, well-known sterilization techniques. In some embodiments, for various oral dosage form excipients such as tablets and capsules, sterility is not required. For example, the United States Pharmacopeia/National Formulary (USP/NF) standard can be sufficient.

In some embodiments, one or more compounds as disclosed herein, or a stereoisomer or mixture of stereoisomers thereof as described herein or a pharmaceutical composition thereof is formulated for ocular administration. In some embodiments, ocular compositions can include, without limitation, one or more of any of the following: viscogens (e.g., carboxymethylcellulose, glycerin, polyvinylpyrrolidone, polyethylene glycol); stabilizers (e.g., Pluronic (triblock copolymers), cyclodextrins); preservatives (e.g., benzalkonium chloride, EDTA, SofZia (boric acid, propylene glycol, sorbitol, and zinc chloride; Alcon Laboratories, Inc.), Purite (stabilized oxychloro complex; Allergan, Inc.)).

In some embodiments, one or more compounds as disclosed herein, or a stereoisomer or mixture of stereoisomers thereof as described herein or a pharmaceutical composition thereof is formulated for topical administration to the skin or mucosa (e.g., dermally or transdermally). In some embodiments, topical compositions can include ointments and creams. In some embodiments, ointments are semisolid preparations that are typically based on petrolatum or other petroleum derivatives. In some embodiments, creams containing the selected active agent are typically viscous liquid or semisolid emulsions, often either oil-in-water or water-in-oil. For example, cream bases are typically water-washable, and contain an oil phase, an emulsifier and an aqueous phase. For example, the oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. In some embodiments, the emulsifier in a cream formulation is generally a nonionic, anionic, cationic or amphoteric surfactant. In some embodiments, as with other carriers or vehicles, an ointment base should be inert, stable, nonirritating and non-sensitizing.

In any of the foregoing embodiments, pharmaceutical compositions as described herein can include one or more one or more of the following: lipids, interbilayer crosslinked multilamellar vesicles, biodegradable poly(D,L-lactic-co-glycolic acid) (PLGA)-based or poly anhydride-based nanoparticles or microparticles, and nanoporous particle-supported lipid bilayers.

The amount of the compound in a pharmaceutical composition or formulation can vary within the full range employed by those skilled in the art. Typically, the formulation will contain, on a weight percent (wt %) basis, from about 0.01-99.99 wt % of a compound of this disclosure based on the total formulation, with the balance being one or more suitable pharmaceutical excipients. In one embodiment, the compound is present at a level of about 1-80 wt %. Representative pharmaceutical formulations are described below.

Formulation Example 1—Tablet formulation

The following ingredients are mixed intimately and pressed into single scored tablets.

| | Quantity per |
|---|---|
| Ingredient | tablet, mg |
| compound of this disclosure | 400 |
| cornstarch | 50 |
| croscarmellose sodium | 25 |
| lactose | 120 |
| magnesium stearate | 5 |

### Formulation Example 2—Capsule Formulation

The following ingredients are mixed intimately and loaded into a hard-shell gelatin capsule

| | Quantity per |
|---|---|
| Ingredient | capsule, mg |
| compound of this disclosure | 200 |
| lactose, spray-dried | 148 |
| magnesium stearate | 2 |

### Formulation Example 3—Suspension Formulation

The following ingredients are mixed to form a suspension for oral administration.

| Ingredient | Amount | |
|---|---|---|
| compound of this disclosure | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| coloring | 0.5 | mg |

| | |
|---|---|
| distilled water | q.s. to 100 mL |

### Formulation Example 4—Injectable Formulation

The following ingredients are mixed to form an injectable formulation.

| Ingredient | Amount |
|---|---|
| compound of this disclosure | 0.2 mg-20 mg |
| sodium acetate buffer solution, 0.4M | 2.0 mL |
| HCl (1N) or NaOH (1N) | q.s. to suitable pH |
| water (distilled, sterile) | q.s. to 20 mL |

### Formulation Example 5—Suppository Formulation

A suppository of total weight 2.5 g is prepared by mixing the compound of this disclosure with Witepsol® H-15 (triglycerides of saturated vegetable fatty acid; Riches-Nelson, Inc., New York), and has the following composition:

| Ingredient | Amount |
|---|---|
| compound of this disclosure | 500 mg |
| Witepsol ® H-15 | balance |

In some embodiments, the dosage for one or more compounds as disclosed herein, or a stereoisomer or mixture of stereoisomers thereof, is determined based on a multiple factors including, but not limited to, type, age, weight, sex, medical condition of the patient, severity of the medical condition of the patient, route of administration, and activity of the compound or pharmaceutically acceptable s salt, stereoisomer, mixture of stereoisomers, or solvate thereof. In some embodiments, proper dosage for a particular situation can be determined by one skilled in the medical arts. In some embodiments, the total daily dosage may be divided and administered in portions throughout the day or by means providing continuous delivery.

In some embodiments, one or more compounds as disclosed herein, or a stereoisomer or mixture of stereoisomers thereof, is administered at a dose from about 0.01 to about 1000 mg. For example, from about 0.1 to about 30 mg, about 10 to about 80 mg, about 0.5 to about 15 mg, about 50 mg to about 200 mg, about 100 mg to about 300 mg, about 200 to about 400 mg, about 300 mg to about 500 mg, about 400 mg to about 600 mg, about 500 mg to about 800 mg, about 600 mg to about 900 mg, or about 700 mg to about 1000 mg. In some embodiments, the dose is a therapeutically effective amount.

In some embodiments, one or more compounds as disclosed herein, or a stereoisomer or mixture of stereoisomers thereof as described herein is administered at a dosage of from about 0.0002 mg/Kg to about 100 mg/Kg (e.g., from about 0.0002 mg/Kg to about 50 mg/Kg; from about 0.0002 mg/Kg to about 25 mg/Kg; from about 0.0002 mg/Kg to about 10 mg/Kg; from about 0.0002 mg/Kg to about 5 mg/Kg; from about 0.0002 mg/Kg to about 1 mg/Kg; from about 0.0002 mg/Kg to about 0.5 mg/Kg; from about 0.0002 mg/Kg to about 0.1 mg/Kg; from about 0.001 mg/Kg to about 50 mg/Kg; from about 0.001 mg/Kg to about 25 mg/Kg; from about 0.001 mg/Kg to about 10 mg/Kg; from about 0.001 mg/Kg to about 5 mg/Kg; from about 0.001 mg/Kg to about 1 mg/Kg; from about 0.001 mg/Kg to about 0.5 mg/Kg; from about 0.001 mg/Kg to about 0.1 mg/Kg; from about 0.01 mg/Kg to about 50 mg/Kg; from about 0.01 mg/Kg to about 25 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 50 mg/Kg; from about 0.1 mg/Kg to about 25 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg). In some embodiments, one or more compounds as disclosed herein, or a stereoisomer or mixture of stereoisomers thereof as described herein is administered as a dosage of about 100 mg/Kg.

In some embodiments, the foregoing dosages of one or more compounds as disclosed herein, or a stereoisomer or mixture of stereoisomers thereof, can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of one or more compounds as disclosed herein, or a stereoisomer or mixture of stereoisomers thereof as described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, one or more compounds as disclosed herein, or a stereoisomer or mixture of stereoisomers thereof is administered to a patient for a period of time followed by a separate period of time where administration of one or more compounds as disclosed herein, or a stereoisomer or mixture of stereoisomers thereof is stopped. In some embodiments, one or more compounds as disclosed herein, or a stereoisomer or mixture of stereoisomers thereof is administered for a first period and a second period following the first period, with administration stopped during the second period, followed by a third period where administration of one or more compounds as disclosed herein, or a stereoisomer or mixture of stereoisomers thereof is started and then a fourth period following the third period where administration is stopped. For example, the period of administration of one or more compounds as disclosed herein, or a stereoisomer or mixture of stereoisomers thereof followed by a period where administration is stopped is repeated for a determined or undetermined period of time. In some embodiments, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In some embodiments, one or more compounds as disclosed herein, or a stereoisomer or mixture of stereoisomers thereof, is orally administered to the patient one or more times per day (e.g., one time per day, two times per day, three times per day, four times per day per day or a single daily dose).

In some embodiments, one or more compounds as disclosed herein, or a stereoisomer or mixture of stereoisomers thereof, is administered by parenteral administration to the patient one or more times per day (e.g., 1 to 4 times, one time per day, two times per day, three times per day, four times per day or a single daily dose).

In some embodiments, one or more compounds as disclosed herein, or a stereoisomer or mixture of stereoisomers thereof, is administered by parenteral administration to the patient weekly.

### Methods of Treatment

In some embodiments, this disclosure provides methods for treating a subject (e.g., a human) having a disease, disorder, or condition in which inhibition of one or more calcitonin receptor and/or amylin receptor is beneficial for the treatment of the underlying pathology and/or symptoms and/or progression of the disease, disorder, or condition. In some embodiments, the methods provided herein can include treating one or more conditions associated, co-morbid or sequela with any one or more of the conditions provided herein.

Provided herein is a method for treating a calcitonin receptor and/or an amylin receptor associated disease or disorder, the method comprising administering to a subject in need thereof an effective amount of a compound disclosed herein (e.g., a compound of Formula I, or any subformula thereof or a pharmaceutically acceptable salt, stereoisomer, mixture of stereoisomers, or solvate thereof), or a pharmaceutical composition as disclosed herein. Also provided herein are methods for treating or preventing a calcitonin receptor and/or an amylin receptor associated disease or disorder in a subject in need thereof, the method comprising administering to a subject in need thereof a therapeutically effective amount of a compound disclosed herein (e.g., a compound of Formula I or any subformula thereof, or a pharmaceutically acceptable salt, stereoisomer, mixture of stereoisomers, or solvate thereof), or a pharmaceutical composition thereof.

In some embodiments, the calcitonin receptor and/or amylin receptor associated disease or disorder is a bone disorder, a metabolic disorder, pain, a neurodegenerative disease or disorder, a cardiovascular disease, or other disease or disorder as described herein.

In some embodiments, the disease or disorder includes, but is not limited to type 1 diabetes mellitus, type 2 diabetes mellitus, early onset type 2 diabetes mellitus, idiopathic type 1 diabetes mellitus (Type 1b), youth-onset atypical diabetes (YOAD), maturity onset diabetes of the young (MODY), latent autoimmune diabetes in adults (LADA), obesity, weight gain from use of other agents, gout, excessive sugar craving, hypertriglyceridemia, dyslipidemia, malnutrition-related diabetes, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn's disease, short bowel syndrome, Parkinson's, Alzheimer's disease, impaired cognition, schizophrenia, and Polycystic Ovary Syndrome (PCOS).

In some embodiments, the disease or disorder includes, but is not limited to type 2 diabetes mellitus, early onset type 2 diabetes mellitus, obesity, weight gain from use of other agents, gout, excessive sugar craving, hypertriglyceridemia, dyslipidemia, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), short bowel syndrome, Parkinson's disease, Polycystic Ovary Syndrome (PCOS), or any combination thereof.

In some embodiments, the disease or disorder includes, but is not limited to type 2 diabetes mellitus, early onset type 2 diabetes mellitus, obesity, weight gain from use of other agents, gout, excessive sugar craving, hypertriglyceridemia, dyslipidemia, gestational diabetes, adipocyte dysfunction, visceral adipose deposition, myocardial infarction, peripheral arterial disease, stroke, transient ischemic attacks, hyperglycemia, post-prandial lipemia, metabolic acidosis, ketosis, hyperinsulinemia, impaired glucose metabolism, insulin resistance, hepatic insulin resistance, chronic renal failure, syndrome X, angina pectoris, diabetic nephropathy, impaired glucose tolerance, diabetic neuropathy, diabetic retinopathy, skin and connective tissue disorders, foot ulcerations, or any combination thereof.

In some embodiments, the compounds and pharmaceutical compositions and methods for treating a patient described herein induce one or more of blood glucose reduction (e.g., reduce blood glucose levels), reduce blood hemoglobin A1c (HbA1c) levels, promote insulin synthesis, stimulate insulin secretion, increase the mass of β-cells, modulate gastric acid secretion, modulate gastric emptying, decrease the body mass index (BMI), and/or decrease glucagon production (e.g., level). In certain embodiments, the compounds and pharmaceutical compositions and methods for treating a patient described herein stabilize serum glucose and serum insulin levels (e.g., serum glucose and serum insulin concentrations). Also provided herein are methods for modulating glucose or insulin levels in a patient in need of such modulating, the method comprising administering to the patient an effective amount of one or more compounds as disclosed herein, or a stereoisomer or mixture of stereoisomers thereof, or a pharmaceutical composition as disclosed herein.

In some embodiments, provided herein is a method for reducing the risk (e.g., by about at least 20%, at least 30%, at least 40%, at least 50%, at least 60%, at least 70%, or at least 80%) of major adverse cardiovascular events (MACE) in a patient in need thereof, the method comprising administering to the patient an effective amount of one or more compounds as disclosed herein, or a stereoisomer or mixture of stereoisomers thereof, or a pharmaceutical composition as disclosed herein. In certain of these embodiments, the patient is an adult that has been diagnosed with type 2 diabetes (T2D). In certain embodiments, the patient is an adult that has been diagnosed with a heart disease. In certain embodiments, the patient is an adult that has been diagnosed with type 2 diabetes (T2D) and a heart disease. In certain embodiments, the patient is an adult that has type 2 diabetes (T2D). In certain embodiments, the patient is an adult that has a heart disease. In certain embodiments, the patient has type 2 diabetes (T2D) and a heart disease.

In some embodiments, the calcitonin receptor and/or amylin receptor associated disease or disorder is a bone disorder, including, but not limited to, osteoporosis, Paget's disease, hypercalcemia, Sudeck's atrophy, polystatic fibrous displasia, intersemocostoclavicular ossification, osteogenesis imperfecta, osteopenia, periodontal disease or defect, osteolytic bone disease, metastatic bone disorder, or bone loss resulting from a malignancy, autoimmune arthritides, a breakage or fracture, or immobility or disuse.

In some embodiments, the calcitonin receptor and/or amylin receptor associated disease or disorder is pain, including, but not limited to, osteopathic pain, phantom limb pain, general pain, hyperalgesia, or pain associated with diabetic neuropathy.

In some embodiments, the calcitonin receptor and/or amylin receptor associated disease or disorder is a neurodegenerative disease or disorder, including, but not limited to, Alzheimer's disease.

In some embodiments, the calcitonin receptor and/or amylin receptor associated disease or disorder is a metabolic disorder, including, but not limited to, non-alcoholic fatty liver disease (NAFLD), non-alcoholic steatohepatitis (NASH), insulin dependent diabetes, non-insulin dependent diabetes, impaired glucose tolerance, obesity, syndrome X, or other diabetic complication.

In some embodiments, the calcitonin receptor and/or amylin receptor associated disease or disorder is include primary or secondary hyperthyroidism, endocrine disorder, conditions associated with inhibiting gastric secretion, gastrointestinal disorders, renal osteodystrophy, or male infertility.

In some embodiments, a compound disclosed herein (e.g., a compound of Formula I, or a pharmaceutically acceptable salt, stereoisomer, mixture of stereoisomers, or solvate thereof), or a pharmaceutical composition as provided herein is useful to alleviate insulin suppression in pancreatic tissue.

In some embodiments, a compound disclosed herein (e.g., a compound of Formula I, or a pharmaceutically acceptable salt, stereoisomer, mixture of stereoisomers, or solvate thereof), or a pharmaceutical composition as provided herein is useful to treat alleviate insulin resistance.

In some embodiments, a compound disclosed herein (e.g., a compound of Formula I, or a pharmaceutically acceptable salt, stereoisomer, mixture of stereoisomers, or solvate thereof), or a pharmaceutical composition as provided herein is useful to treat impaired glucose tolerance.

In some embodiments, a compound disclosed herein (e.g., a compound of Formula I, or a pharmaceutically acceptable salt, stereoisomer, mixture of stereoisomers, or solvate thereof), or a pharmaceutical composition as provided herein is useful to treat obesity and symptoms thereof.

In some embodiments, provided is a method for reducing body fat or body fat gain, comprising administering to a subject in need thereof a therapeutically effective amount of a compound disclosed herein (e.g., a compound of Formula I, or a pharmaceutically acceptable salt, stereoisomer, mixture of stereoisomers, or solvate thereof), or a pharmaceutical composition as provided herein.

In some embodiments, provided is a method of altering a body composition of a subject in need of treatment, wherein body fat is reduced and lean body mass is maintained or increased, comprising administering to the subject a therapeutically effective amount of a compound disclosed herein (e.g., a compound of Formula I, or a pharmaceutically acceptable salt, stereoisomer, mixture of stereoisomers, or solvate thereof), or a pharmaceutical composition as provided herein.

In some embodiments, provided is a method for reducing body weight in a subject in need of, comprising administering to the subject a therapeutically effective amount of a compound disclosed herein (e.g., a compound of Formula I, or a pharmaceutically acceptable salt, stereoisomer, mixture of stereoisomers, or solvate thereof), or a pharmaceutical composition as provided herein.

In some embodiments, provided is a method for reducing caloric intake in a subject in need of reduction thereof, comprising administering to the subject a therapeutically effective amount of a compound disclosed herein (e.g., a compound of Formula I, or a pharmaceutically acceptable salt, stereoisomer, mixture of stereoisomers, or solvate thereof), or a pharmaceutical composition as provided herein.

In some embodiments, provided is a method for reducing body fat or body fat gain in a subject in need of treatment while maintaining or increasing lean body mass, comprising administering to the subject a therapeutically effective amount of a compound disclosed herein (e.g., a compound of Formula I, or a pharmaceutically acceptable salt, stereoisomer, mixture of stereoisomers, or solvate thereof), or a pharmaceutical composition as provided herein.

In some embodiments, a compound disclosed herein (e.g., a compound of Formula I, or a pharmaceutically acceptable salt, stereoisomer, mixture of stereoisomers, or solvate thereof), or a pharmaceutical composition as provided herein is useful to treat hypertension.

In some embodiments, a compound disclosed herein (e.g., a compound of Formula I, or a pharmaceutically acceptable salt, stereoisomer, mixture of stereoisomers, or solvate thereof), or a pharmaceutical composition as provided herein is useful to treat essential hypertension.

In some embodiments, a compound disclosed herein (e.g., a compound of Formula I, or a pharmaceutically acceptable salt, stereoisomer, mixture of stereoisomers, or solvate thereof), or a pharmaceutical composition as provided herein is useful to treat a subject suffering from hypertension and hyperamylinemia.

In some embodiments, provided is a method for treating hyperinsulinemia, comprising administering to a subject in need thereof a therapeutically effective amount of a compound disclosed herein (e.g., a compound of Formula I, or a pharmaceutically acceptable salt, stereoisomer, mixture of stereoisomers, or solvate thereof), or a pharmaceutical composition as provided herein.

In some embodiments, provided is a method for treating a hypertensive, insulin-resistant subject suffering from coronary artery disease and having hyperamylinemia or hyperinsulinemia, comprising administering to a subject in need thereof a therapeutically effective amount of a compound disclosed herein (e.g., a compound of Formula I, or a pharmaceutically acceptable salt, stereoisomer, mixture of stereoisomers, or solvate thereof), or a pharmaceutical composition as provided herein.

In some embodiments, provided is a method for decreasing basal and submaximally stimulated rates of glycogen synthesis in a subject, comprising administering to a subject in need thereof a therapeutically effective amount of a compound disclosed herein (e.g., a compound of Formula I, or a pharmaceutically acceptable salt, stereoisomer, mixture of stereoisomers, or solvate thereof), or a pharmaceutical composition as provided herein.

In some embodiments, provided is a method for decreasing the rate of incorporation of glucose into glycogen in muscle tissue of a subject, comprising administering to a subject in need thereof a therapeutically effective amount of a compound disclosed herein (e.g., a compound of Formula I, or a pharmaceutically acceptable salt, stereoisomer, mixture of stereoisomers, or solvate thereof), or a pharmaceutical composition as provided herein.

In some embodiments, provided is a method for treating obesity and hypertension, and the lipid disorders and atherosclerosis associated therewith, in a subject, comprising administering to a subject in need thereof a therapeutically effective amount of a compound disclosed herein (e.g., a compound of Formula I, or a pharmaceutically acceptable salt, stereoisomer, mixture of stereoisomers, or solvate thereof), or a pharmaceutical composition as provided herein.

In some embodiments, a compound disclosed herein (e.g., a compound of Formula I, or a pharmaceutically acceptable salt, stereoisomer, mixture of stereoisomers, or solvate thereof), or a pharmaceutical composition as provided herein is useful to modulate renin activity in a subject in need thereof.

In some embodiments, provided is a method for treating or preventing the development of cardiac failure, in a subject, comprising administering to a subject in need thereof a therapeutically effective amount of a compound disclosed herein (e.g., a compound of Formula I, or a pharmaceutically acceptable salt, stereoisomer, mixture of stereoisomers, or solvate thereof), or a pharmaceutical composition as provided herein.

In some embodiments, a compound disclosed herein (e.g., a compound of Formula I, or a pharmaceutically acceptable salt, stereoisomer, mixture of stereoisomers, or solvate thereof), or a pharmaceutical composition as provided herein is useful to beneficially regulate gastrointestinal motility in a subject in need thereof. In some embodiments, the beneficial regulation of gastrointestinal motility comprises delaying gastric emptying.

In some embodiments, a compound disclosed herein (e.g., a compound of Formula I, or a pharmaceutically acceptable salt, stereoisomer, mixture of stereoisomers, or solvate thereof), or a pharmaceutical composition as provided herein is useful to treat postprandial hyperglycemia in a subject in need thereof.

### Obesity

In some embodiments, the condition, disease or disorder is obesity and conditions, diseases or disorders that are associated with or related to obesity. Non-limiting examples of obesity and obesity related conditions include symptomatic obesity, simple obesity, childhood obesity, morbid obesity, and abdominal obesity (central obesity characterized by abdominal adiposity). Non-limiting examples of symptomatic obesity include endocrine obesity (e.g., Cushing syndrome, hypothyroidism, insulinoma, obese type II diabetes, pseudohypoparathyroidism, hypogonadism), hypothalamic obesity, hereditary obesity (e.g., Prader-Willi syndrome, Laurence-Moon-Biedl syndrome), and drug-induced obesity (e.g., steroid, phenothiazine, insulin, sulfonylurea agent, or β-blocker-induced obesity).

In some embodiments, the condition, disease or disorder is associated with obesity. Examples of such conditions, diseases or disorders include, without limitation, glucose tolerance disorders, diabetes (e.g., type 2 diabetes, obese diabetes), lipid metabolism abnormality, hyperlipidemia, hypertension, cardiac failure, hyperuricemia, gout, fatty liver (including non-alcoholic steatohepatitis (NASH)), coronary heart disease (e.g., myocardial infarction, angina pectoris), cerebral infarction (e.g., brain thrombosis, transient cerebral ischemic attack), bone or articular disease (e.g., knee osteoarthritis, hip osteoarthritis, spondylitis deformans, lumbago), sleep apnea syndrome, obesity hypoventilation syndrome (Pickwickian syndrome), menstrual disorder (e.g., abnormal menstrual cycle, abnormality of menstrual flow and cycle, amenorrhea, abnormal catamenial symptom), visceral obesity syndrome, and metabolic syndrome. In some embodiments, the chemical compound and pharmaceutical compositions described herein can be used to treat patients exhibiting symptoms of both obesity and insulin deficiency.

### Diabetes

In some embodiments, the condition, disease or disorder is diabetes. Non-limiting examples of diabetes include type 1 diabetes mellitus, type 2 diabetes mellitus (e.g., diet-treated type 2-diabetes, sulfonylurea-treated type 2-diabetes, a far-advanced stage type 2-diabetes, long-term insulin-treated type 2-diabetes), diabetes mellitus (e.g., non-insulin-dependent diabetes mellitus, insulin-dependent diabetes mellitus), gestational diabetes, obese diabetes, autoimmune diabetes, and borderline type diabetes. In some embodiments, the condition, disease or disorder is type 2 diabetes mellitus (e.g., diet-treated type 2-diabetes, sulfonylurea-treated type 2-diabetes, a far-advanced stage type 2-diabetes, long-term insulin-treated type 2-diabetes).

Provided herein is a method of treating a diabetes mellitus in a patient, the method comprising (a) determining that the patient has type 2 diabetes mellitus, and (b) administering to the patient a therapeutically effective amount of one or more compounds as disclosed herein, or a stereoisomer or mixture of stereoisomers thereof, or a pharmaceutical composition as disclosed herein.

Provided herein is a method for treating type 2 diabetes mellitus in a patient, the method comprising administering to a patient identified or diagnosed as having type 2 diabetes mellitus a therapeutically effective amount of one or more compounds as disclosed herein, or a stereoisomer or mixture of stereoisomers thereof, or a pharmaceutical composition as disclosed herein.

Also provided herein is a method of treating type 2 diabetes mellitus in a patient in need thereof, the method comprising administering to the patient a therapeutically effective amount of one or more compounds as disclosed herein, or a stereoisomer or mixture of stereoisomers thereof, or a pharmaceutical composition as disclosed herein.

In some embodiments, the compounds and pharmaceutical compositions and methods for treating a patient with a condition, disease, or disorder (e.g., type 2 diabetes mellitus) described herein reduce fasting plasma glucose levels. In some embodiments, the compounds and pharmaceutical compositions and methods for treating a patient with a condition, disease, or disorder (e.g., type 2 diabetes mellitus) described herein reduce non-fasting plasma glucose levels. In some embodiments, the compounds and pharmaceutical compositions and methods for treating a patient with a condition, disease, or disorder (e.g., type 2 diabetes mellitus) described herein reduce HbA1c levels. In some embodiments, the compounds and pharmaceutical compositions and methods for treating a patient with a condition, disease, or disorder (e.g., type 2 diabetes mellitus) described herein reduce glucagon levels. In some embodiments, the compounds and pharmaceutical compositions and methods for treating a patient with a condition, disease, or disorder (e.g., type 2 diabetes mellitus) described herein increase insulin levels. In some embodiments, the compounds and pharmaceutical compositions and methods for treating a patient with a condition, disease, or disorder (e.g., type 2 diabetes mellitus) described herein reduce BMI.

In some embodiments, a reduction in fasting plasma glucose levels of about 5% to about 95% indicates treatment of type 2 diabetes mellitus. In some embodiments, a reduction in fasting plasma glucose levels of about 15% to about 80% indicates treatment of type 2 diabetes mellitus. In some embodiments, a reduction in fasting plasma glucose levels of about 25% to about 60% indicates treatment of type 2 diabetes mellitus. In some embodiments, a reduction in fasting plasma glucose levels to about or below 126 mg/dL, about or below 110 mg/dL, or about or below 90 mg/dL indicates treatment of the type 2 diabetes mellitus.

In some embodiments, a reduction in non-fasting plasma glucose levels of about 5% to about 95% indicates treatment of type 2 diabetes mellitus. In some embodiments, a reduction in non-fasting plasma glucose levels of about 15% to about 80% indicates treatment of type 2 diabetes mellitus. In some embodiments, a reduction in non-fasting plasma glucose levels of about 25% to about 60% indicates treatment of type 2 diabetes mellitus. In some embodiments, a reduction in non-fasting plasma glucose levels to about or below 200 mg/dL, about or below 150 mg/dL, or about or below 130 mg/dL indicates treatment of type 2 diabetes mellitus.

In some embodiments, a reduction in HbA1c levels of about 5% to about 95% indicates treatment of type 2 diabetes mellitus. In some embodiments, a reduction in HbA1c levels of about 15% to about 80% indicates treatment of type 2 diabetes mellitus. In some embodiments, a reduction in HbA1c levels of about 25% to about 60% indicates treatment of type 2 diabetes mellitus. In some embodiments, reduction in HbA1c levels to about or below 6.5%, about or below 6.0%, or about or below 5.0% indicates treatment of type 2 diabetes mellitus.

In some embodiments, a reduction in glucagon levels of about 5% to about 95% indicates treatment of type 2 diabetes mellitus. In some embodiments, a reduction in glucagon levels of about 15% to about 80% indicates treatment of type 2 diabetes mellitus. In some embodiments, a reduction in glucagon levels of about 25% to about 60% indicates treatment of type 2 diabetes mellitus. In some embodiments, an increase in insulin levels of about 5% to about 95% indicates treatment of type 2 diabetes mellitus. In some embodiments, an increase in insulin levels of about 15% to about 80% indicates treatment of type 2 diabetes mellitus. In some embodiments, an increase in insulin levels of about 25% to about 60% indicates treatment of type 2 diabetes mellitus.

In some embodiments, a reduction in BMI of about 5% to about 95% indicates treatment of type 2 diabetes mellitus. In some embodiments, a reduction in BMI of about 15% to about 80% indicates treatment of the type 2 diabetes mellitus. In some embodiments, a reduction in BMI of about 25% to about 60% indicates treatment of type 2 diabetes mellitus. In some embodiments, a reduction in BMI of about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% indicates treatment of type 2 diabetes mellitus. In some embodiments, a reduction in BMI to about or below 40, about or below 30, or about or below 20 indicates treatment of type 2 diabetes mellitus.

In some embodiments, the condition, disease or disorder is associated with diabetes (e.g., a complication of diabetes). Non-limiting examples of disorders associated with diabetes include obesity, obesity-related disorders, metabolic syndrome, neuropathy, nephropathy (e.g., diabetic nephropathy), retinopathy, diabetic cardiomyopathy, cataract, macroangiopathy, osteopenia, hyperosmolar diabetic coma, infectious disease (e.g., respiratory infection, urinary tract infection, gastrointestinal infection, dermal soft tissue infections, inferior limb infection), diabetic gangrene, xerostomia, hypacusis, cerebrovascular disorder, diabetic cachexia, delayed wound healing, diabetic dyslipidemia peripheral blood circulation disorder, cardiovascular risk factors. (e.g., coronary artery disease, peripheral artery disease, cerebrovascular disease, hypertension, and risk factors related to unmanaged cholesterol and/or lipid levels, and/or inflammation), NASH, bone fracture, and cognitive dysfunction

Other non-limiting examples of disorders related to diabetes include pre-diabetes, hyperlipidemia (e.g., hypertriglyceridemia, hypercholesterolemia, high LDL-cholesterolemia, low HDL-cholesterolemia, postprandial hyperlipemia), metabolic syndrome (e.g., metabolic disorder where activation of GLP-1R is beneficial, metabolic syndrome X), hypertension, impaired glucose tolerance (IGT), insulin resistance, and sarcopenia.

In some embodiments, the condition, disease or disorder is diabetes and obesity (diabesity). In some embodiments, the compounds described herein are also useful in improving the therapeutic effectiveness of metformin.

### Disorders of Metabolically Important Tissues

In some embodiments, the condition, disease or disorder is a disorder of a metabolically important tissue. Non-limiting examples of metabolically important tissues include liver, fat, pancreas, kidney, and gut.

In some embodiments, the condition, disease or disorder is a fatty liver disease. Fatty liver diseases include, but are not limited to, non-alcoholic fatty acid liver disease (NAFLD), steatohepatitis, non-alcoholic steatohepatitis (NASH), fatty liver disease resulting from hepatitis, fatty liver disease resulting from obesity, fatty liver disease resulting from diabetes, fatty liver disease resulting from insulin resistance, fatty liver disease resulting from hypertriglyceridemia, Abetalipoproteinemia, glycogen storage diseases, Weber-Christian disease, Wolman's disease, acute fatty liver of pregnancy, and lipodystrophy.

Non-alcoholic fatty liver disease (NAFLD) represents a spectrum of disease occurring in the absence of alcohol abuse and is typically characterized by the presence of steatosis (fat in the liver). NAFLD is believed to be linked to a variety of conditions, e.g., metabolic syndrome (including obesity, diabetes and hypertriglyceridemia) and insulin resistance. It can cause liver disease in adults and children and may ultimately lead to cirrhosis (Skelly et al., J Hepatol 2001; 35: 195-9; Chitturi et al., Hepatology 2002; 35(2):373-9). The severity of NAFLD ranges from the relatively benign isolated predominantly macrovesicular steatosis (i.e., nonalcoholic fatty liver or NAFL) to non-alcoholic steatohepatitis (NASH) (Angulo et al., J Gastroenterol Hepatol 2002; 17 Suppl: S186-90). In some embodiments, the patient is a pediatric patient. The term “pediatric patient” as used herein refers to a patient under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W. B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994. In some embodiments, a pediatric patient is from birth through the first 28 days of life, from 29 days of age to less than two years of age, from two years of age to less than 12 years of age, or 12 years of age through 21 years of age (up to, but not including, the twenty-second birthday). In some embodiments, a pediatric patient is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age. In some embodiments, the patient is an adult patient.

Other non-limiting examples of disorders in metabolically important tissues include joint disorders (e.g., osteoarthritis, secondary osteoarthritis), steatosis (e.g. in the liver); gall stones; gallbladder disorders; gastroesophageal reflux; sleep apnea; hepatitis; fatty liver; bone disorder characterized by altered bone metabolism, such as osteoporosis, including post-menopausal osteoporosis, poor bone strength, osteopenia, Paget's disease, osteolytic metastasis in cancer patients, osteodistrophy in liver disease and the altered bone metabolism caused by renal failure or hemodialysis, bone fracture, bone surgery, aging, pregnancy, protection against bone fractures, and malnutrition polycystic ovary syndrome; renal disease (e.g., chronic renal failure, glomerulonephritis, glomerulosclerosis, nephrotic syndrome, hypertensive nephrosclerosis, end-stage renal disease); muscular dystrophy, angina pectoris, acute or chronic diarrhea, testicular dysfunction, respiratory dysfunction, frailty, sexual dysfunction (e.g., erectile dysfunction), and geriatric syndrome. In some embodiments, the compounds and pharmaceutical compositions described herein can be used for treating surgical trauma by improving recovery after surgery and/or by preventing the catabolic reaction caused by surgical trauma.

### Cardiovascular and Vascular Diseases

In some embodiments, the disease or disorder is a cardiovascular disease. Non-limiting examples of cardiovascular disease include congestive heart failure, atherosclerosis, arteriosclerosis, coronary heart disease, coronary artery disease, congestive heart failure, coronary heart disease, hypertension, cardiac failure, cerebrovascular disorder (e.g., cerebral infarction), vascular dysfunction, myocardial infarction, elevated blood pressure (e.g., 130/85 mm Hg or higher), and prothrombotic state (exemplified by high fibrinogen or plasminogen activator inhibitor in the blood).

In some embodiments, the disease or disorder is related to a vascular disease. Non-limiting examples of vascular diseases include peripheral vascular disease, macrovascular complications (e.g., stroke), vascular dysfunction, peripheral artery disease, abdominal aortic aneurysm, carotid artery disease, cerebrovascular disorder (e.g., cerebral infarction), pulmonary embolism, chronic venous insufficiency, critical limb ischemia, retinopathy, nephropathy, and neuropathy.

### Neurological Diseases

In some embodiments, the disease or disorder is a neurological disorder (e.g., neurodegenerative disorder) or a psychiatric disorder. Non-limiting examples of neurological disorders include brain insulin resistance, mild cognitive impairment (MCI), Alzheimer's disease (AD), Parkinson's disease (PD), anxiety, dementia (e.g., senile dementia), traumatic brain injury, Huntington's chores, tardive dyskinesia, hyperkinesia, mania, Morbus Parkinson, steel-Richard syndrome, Down's syndrome, myasthenia gravis, nerve trauma, brain trauma, vascular amyloidosis, cerebral hemorrhage I with amyloidosis, brain inflammation, Friedrich's ataxia, acute confusion disorder, amyotrophic lateral sclerosis (ALS), glaucoma, and apoptosis-mediated degenerative diseases of the central nervous system (e.g., Creutzfeld-Jakob Disease, bovine spongiform encephalopathy (mad cow disease), and chronic wasting syndrome). See, e.g., US2006/0275288A1.

Non-limiting examples of psychiatric disorders include drug dependence/addiction (narcotics and amphetamines and attention deficit/hyperactivity disorder (ADHD). The compounds and pharmaceutical compositions described herein can be useful in improving behavioral response to addictive drugs, decreasing drug dependence, prevention drug abuse relapse, and relieving anxiety caused by the absence of a given addictive substance. See, e.g., US2012/0021979A1.

In some embodiments, the compounds and pharmaceutical compositions described herein are useful in improving learning and memory by enhancing neuronal plasticity and facilitation of cellular differentiation, and also in preserving dopamine neurons and motor function in Morbus Parkinson.

### Insulin-Related Conditions and Disorders

In some embodiments, the disease or disorder is impaired fasting glucose (IFG), impaired fasting glycemia (IFG), hyperglycemia, insulin resistance (impaired glucose homeostasis), hyperinsulinemia, elevated blood levels of fatty acids or glycerol, a hypoglycemic condition, insulin resistant syndrome, paresthesia caused by hyperinsulinemia, hyperlipidemia, hypercholesteremia, impaired wound healing, leptin resistance, glucose intolerance, increased fasting glucose, dyslipidemia (e.g., hyperlipidemia, atherogenic dyslipidemia characterized by high triglycerides and low HDL cholesterol), glucagonoma, hyperprolactinemia, hypoglycemia (e.g., nighttime hypoglycemia), and concomitant comatose endpoint associated with insulin.

In some embodiments, the compounds and pharmaceutical compositions described herein can reduce or slow down the progression of borderline type, impaired fasting glucose or impaired fasting glycemia into diabetes.

### Autoimmune Disorders

In some embodiments, the disease or disorder is an autoimmune disorder. Non-limiting examples of autoimmune disorders include multiple sclerosis, experimental autoimmune encephalomyelitis, autoimmune disorder is associated with immune rejection, graft versus host disease, uveitis, optic neuropathies, optic neuritis, transverse myelitis, inflammatory bowel disease, rheumatoid arthritis, ankylosing spondylitis, systemic lupus erythematosus, myasthenia gravis, and Graves' disease. See, e.g., US20120148586A1.

### Stomach and Intestine-Related Disorders

In some embodiments, the disease or disorder is a stomach or intestine related disorder. Non-limiting examples of these disorders include ulcers of any etiology (e.g. peptic ulcers, Zollinger-Ellison syndrome, drug-induced ulcers, ulcers related to infections or other pathogens), digestion disorders, malabsorption, short bowel syndrome, cul-de-sac syndrome, inflammatory bowel diseases (Crohn's disease and ulcerative colitis), celiac sprue, hypogammaglobulinemic sprue, chemotherapy and/or radiation therapy-induced mucositis and diarrhea, gastrointestinal inflammation, short bowel syndrome, colitis ulcerosa, gastric mucosal injury (e.g., gastric mucosal injury caused by aspirin), small intestinal mucosal injury, and cachexia (e.g., cancerous cachexia, tuberculous cachexia, cachexia associated with blood disease, cachexia associated with endocrine disease, cachexia associated with infectious disease, and cachexia caused by acquired immunodeficiency syndrome).

### Body Weight

In some embodiments, the compounds and pharmaceutical compositions described herein can be used to reduce body weight (e.g., excess body weight), prevent body weight gain, induce weight loss, decrease body fat, or reduce food intake in a patient (e.g., a patient in need thereof). In some embodiments, the weight increase in a patient may be attributed to excessive ingestion of food or unbalanced diets, or may be weight increase derived from a concomitant drug (e.g., insulin sensitizers having a PPARγ agonist-like action, such as troglitazone, rosiglitazone, englitazone, ciglitazone, pioglitazone and the like). In some embodiments, the weight increase may be weight increase before reaching obesity, or may be weight increase in an obese patient. In some embodiments, the weight increase may also be medication-induced weight gain or weight gain subsequent to cessation of smoking.

In some embodiments, the disease or disorder is an eating disorder, such as hyperphagia, binge eating, bulimia, or compulsive eating.

### Inflammatory Diseases

In some embodiments, the disease or disorder is an inflammatory disorder. Non-limiting examples of inflammatory disorders include chronic rheumatoid arthritis, spondylitis deformans, arthritis deformans, lumbago, gout, post-operational or post-traumatic inflammation, bloating, neuralgia, laryngopharyngitis, cystitis, pneumonia, pancreatitis, enteritis, inflammatory bowel disease (including inflammatory large bowel disease), inflammation in metabolically important tissues including liver, fat, pancreas, kidney and gut, and a proinflammatory state (e.g., elevated levels of proinflammatory cytokines or markers of inflammation-like C-reactive protein in the blood).

### Cancer

In some embodiments, the disease or disorder is cancer. Suitable examples of cancer include breast cancer (e.g., invasive ductal breast cancer, noninvasive ductal breast cancer, inflammatory breast cancer), prostate cancer (e.g., hormone-dependent prostate cancer, hormone-independent prostate cancer), pancreatic cancer (e.g., ductal pancreatic cancer), gastric cancer (e.g., papillary adenocarcinoma, mucous adenocarcinoma, adenosquamous carcinoma), lung cancer (e.g., non-small cell lung cancer, small-cell lung cancer, malignant mesothelioma), colon cancer (e.g., gastrointestinal stromal tumor), rectal cancer (e.g., gastrointestinal stromal tumor), colorectal cancer (e.g., familial colorectal cancer, hereditary non-polyposis colorectal cancer, gastrointestinal stromal tumor), small intestinal cancer (e.g., non-Hodgkin's lymphoma, gastrointestinal stromal tumor), esophageal cancer, duodenal cancer, tongue cancer, pharyngeal cancer (e.g., nasopharyngeal cancer, oropharynx cancer, hypopharyngeal cancer), salivary gland cancer, brain tumor (e.g., pineal astrocytoma, pilocytic astrocytoma, diffuse astrocytoma, anaplastic astrocytoma), neurilemmoma, liver cancer (e.g., primary liver cancer, extrahepatic bile duct cancer), renal cancer (e.g., renal cell cancer, transitional cell cancer of the renal pelvis and ureter), bile duct cancer, endometrial cancer, uterine cervical cancer, ovarian cancer (e.g., epithelial ovarian cancer, extragonadal germ cell tumor, ovarian germ cell tumor, ovarian tumor of low malignant potential), bladder cancer, urethral cancer, skin cancer (e.g., intraocular (ocular) melanoma, Merkel cell carcinoma), hemangioma, malignant lymphoma, malignant melanoma, thyroid cancer (e.g., medullary thyroid cancer), parathyroid cancer, nasal cavity cancer, sinus cancer, bone tumor (e.g., osteosarcoma, Ewing tumor, uterine sarcoma, soft tissue sarcoma), angiofibroma, sarcoma of the retina, penis cancer, testicular tumor, pediatric solid tumor (e.g., Wilms' tumor, childhood kidney tumor), Kaposi's sarcoma, Kaposi's sarcoma caused by AIDS, tumor of maxillary sinus, fibrous histiocytoma, leiomyosarcoma, rhabdomyosarcoma, and leukemia (e.g., acute myeloid leukemia, acute lymphoblastic leukemia).

Hypothalamic-pituitary disorders

In some embodiments, the disease or disorder is related to the hypothalamic-pituitary-gonadal axis. For example, the condition, disease or disorder is related to the hypothalamus-pituitary-ovary axis. In another example, the condition, disease or disorder is related to the hypothalamus-pituitary-testis axis. Hypothalamic-pituitary-gonadal axis diseases include, but are not limited to, hypogonadism, polycystic ovary syndrome, hypothyroidism, hypopituitarism, sexual dysfunction, and Cushing's disease.

In some embodiments, the disease or disorder associated with diabetes is related to the hypothalamic-pituitary-gonadal axis.

### Pulmonary Disease

In some embodiments, the disease or disorder is related to a pulmonary disease. Pulmonary diseases include, but are not limited to, asthma, idiopathic pulmonary fibrosis, pulmonary hypertension, obstructive sleep apnoea-hypopnoea syndrome, and chronic obstructive pulmonary disease (COPD) (e.g., emphysema, chronic bronchitis, and refractory (non-reversible) asthma).

In some embodiments, the disease or disorder associated with diabetes is a pulmonary disease.

### Combination Therapy

In some embodiments, this disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the compounds described herein.

In some embodiments, the methods described herein include administering a compound described herein in combination with one or more of a diet therapy (e.g., dietary monitoring, diet therapy for diabetes), an exercise therapy (e.g., physical activity), blood sugar monitoring, gastric electrical stimulation (e.g., TANTALUS®), and diet modifications.

In some embodiments, one or more compounds as disclosed herein, or a stereoisomer or mixture of stereoisomers thereof can be administered in combination with one or more additional therapeutic agents.

Representative additional therapeutic agents include, but are not limited to, anti-obesity agents, therapeutic agents for diabetes, therapeutic agents for diabetic complications, therapeutic agents for hyperlipidemia, antihypertensive agents, diuretics, chemotherapeutics, immunotherapeutics, anti-inflammatory drugs, antithrombotic agents, anti-oxidants, therapeutic agents for osteoporosis, vitamins, antidementia drugs, erectile dysfunction drugs, therapeutic drugs for urinary frequency or urinary incontinence, therapeutic agents for NAFLD, therapeutic agents for NASH, therapeutic agents for dysuria and anti-emetic agents.

In some embodiments, the one or more additional therapeutic agents include those useful, for example, as anti-obesity agents. Non-limiting examples include monoamine uptake inhibitors (e.g., tramadol, phentermine, sibutramine, mazindol, fluoxetine, tesofensine), serotonin 2C receptor agonists (e.g., lorcaserin), serotonin 6 receptor antagonists, histamine H3 receptor modulator, GABA modulator (e.g., topiramate), including GABA receptor agonists (e.g., gabapentin, pregabalin), neuropeptide Y antagonists (e.g., velneperit), cannabinoid receptor antagonists (e.g., rimonabant, taranabant), ghrelin antagonists, ghrelin receptor antagonists, ghrelin acylation enzyme inhibitors, opioid receptor antagonists (e.g., GSK-1521498), orexin receptor antagonists, melanocortin 4 receptor agonists, 11β-hydroxysteroid dehydrogenase inhibitors (e.g., AZD-4017, BVT-3498, INCB-13739), pancreatic lipase inhibitors (e.g., orlistat, cetilistat), β3 agonists (e.g., N-5984), diacylglycerol acyltransferase 1 (DGAT1) inhibitors, acetylCoA carboxylase (ACC) inhibitors, stearoyl-CoA desaturated enzyme inhibitors, microsomal triglyceride transfer protein inhibitors (e.g., R-256918), sodium-glucose cotransporter 2 (SGLT-2) inhibitors (e.g., JNJ-28431754, dapagliflozin, AVE2268, TS-033, YM543, TA-7284, ASP1941, remogliflozin), NFK inhibitors (e.g., HE-3286), PPAR agonists (e.g., GFT-505, DRF-11605, gemfibrozil and fenofibrate), phosphotyrosine phosphatase inhibitors (e.g., sodium vanadate, trodusquemin), GPR119 agonists (e.g., PSN-821, MBX-2982, APD597), glucokinase activators (e.g., piragliatin, AZD-1656, AZD6370, TTP-355, compounds described in W0006/112549, W0007/028135, W0008/047821, W0008/050821, W0008/136428 and W0008/156757), leptin, leptin derivatives (e.g., metreleptin), leptin resistance improving drugs, CNTF (ciliary neurotrophic factor), BDNF (brain-derived neurotrophic factor), cholecystokinin agonists, amylin preparations (e.g., pramlintide, AC-2307), neuropeptide Y agonists (e.g., PYY3-36, derivatives of PYY3-36, obineptide, TM-30339, TM-30335), oxyntomodulin (OXM) preparations, appetite suppressants (e.g. ephedrine), FGF21 preparations (e.g., animal FGF21 preparations extracted from the pancreas of bovine or swine; human FGF21 preparations genetically synthesized using Escherichia coli or yeast; fragments or derivatives of FGF21), anorexigenic agents (e.g., P-57), human proislet peptide (HIP), farnesoid X receptor (FXR) agonist, phentermine, zonisamide, norepinephrine/dopamine reuptake inhibitor, GDF-15 analog, methionine aminopeptidase 2 (MetAP2) inhibitor, diethylpropion, phendimetrazine, benzphetamine, fibroblast growth factor receptor (FGFR) modulator, and AMP-activated protein kinase (AMPK) activator.

In some embodiments, one or more compounds as disclosed herein, or a stereoisomer or mixture of stereoisomers thereof can be administered in combination with one or more additional therapeutic agents, wherein the additional therapeutic agent is a GLP-1 agonist or exhibits GLP-1 agonist activity.

In some embodiments, the additional therapeutic agent is TTP273, LY2944876 (pegapamodutide), HDM1002, K-757, K-833, retatrutide, IBI362 (mazdutide), cotadutide, AMG133, CT-868, HRS9531, HS-20094, dapiglutide, efinopegdutide, efocipegtrutide, pemvidutide, survodutide, AP026, AZD9550, BGM0504, CT-388, DDO1, DR10624, G3215, GMA106, HEC88473, HZO10, LY3493269, MWN101, NN9487, NN9541, RAY1225, SCO-094, SHR-1816, TB001, VK2735, ZP2929, ecnoglutide, GX-G6, GZR18, HRS-7535, YH14617, avexitide, froniglutide, pegsebrenatide, vurolenatide, JY09, NB1001, Byetalog, GW002, HL08, KN056, SAL0112, SHR2042, VCT220, ZT002, ZYOG1, or utreglutide.

In some embodiments, the additional therapeutic agent is endogenous GLP-1, endogenous glucagon, oxyntomodulin, exendin-4, exenatide, lixisenatide, albiglutide, beinaglutide, dulaglutide, efpeglenatide, langlenatide, liraglutide, semaglutide, taspoglutide, tirzepatide, pegapamodutide, lithium chloride, PF-06882961 (danuglipron), LY3502970 (orforglipron), ECC-5004, GSBR-1290, AZD0186, PF-07081532 (lotiglipron), VCT220, TERN-601, RGT-075, CT-996, MDR-001, SAL0112, XW014, AVE-0010, S4P, or Boc5),

In some embodiments, one or more compounds as disclosed herein, or a stereoisomer or mixture of stereoisomers thereof can be administered in combination with one or more additional therapeutic agents, wherein the additional therapeutic agent is selected from a compound disclosed in WO2021/155841, WO/2018/109607, WO/2018/056453, WO/2019/239319, or WO/2019/239371.

In some embodiments, the one or more additional therapeutic agents include those useful, for example, as anti-diabetic agents. Non-limiting examples include insulin and insulin preparations (e.g., animal insulin preparations extracted from the pancreas of bovine or swine; human insulin preparations genetically synthesized using Escherichia coli or yeast; zinc insulin; protamine zinc insulin; fragment or derivative of insulin (e.g., INS-1), oral insulin preparation, synthetic human insulin), insulin sensitizers (e.g., pioglitazone or a salt thereof), biguanides (e.g., metformin, buformin or a salt thereof (e.g., hydrochloride, fumarate, succinate)), glucagon analogs (e.g., any of glucagon analogs described, e.g., in WO 2010/011439), agents which antagonize the actions of or reduce secretion of glucagon, sulfonylurea agents (e.g., chlorpropamide, tolazamide, gliclazide, glimepiride, tolbutamide, glibenclamide, gliclazide, acetohexamide, glyclopyramide, glybuzole, glyburide), thiazolidinedione agents (e.g. rosiglitazone or pioglitazone), α-glucosidase inhibitors (e.g., voglibose, acarbose, miglitol, emiglitate), insulin secretagogues, such as prandial glucose regulators (sometimes called “short-acting secretagogues”), e.g., meglitinides (e.g. repaglinide and nateglinide), cholinesterase inhibitors (e.g., donepezil, galantamine, rivastigmine, tacrine), NMDA receptor antagonists, dual GLP-1/GIP receptor agonists (e.g., LBT-2000, ZPD1-70), GLP-1R agonists (e.g., exenatide, liraglutide, albiglutide, dulaglutide, abiglutide, taspoglutide, lixisenatide, semaglutide, AVE-0010, S4P and Boc5), and dipeptidyl peptidase IV (DPP-4) inhibitors (e.g., vildagliptin, dutogliptin, gemigliptin, alogliptin, saxagliptin, sitagliptin, linagliptin, berberine, adogliptin, BI1356, GRC8200, MP-513, PF-00734200, PHX1149, SK-0403, ALS2-0426, TA-6666, TS-021, KRP-104, trelagliptin).

In some embodiments, the one or more additional therapeutic agents include those useful, for example, for treating NAFL and NASH. Non-limiting examples include FXR agonists, PF-05221304, a synthetic fatty acid-bile conjugate, an anti-lysyl oxidase homologue 2 (LOXL2) monoclonal antibody, a caspase inhibitor, a MAPK5 inhibitor, a galectin 3 inhibitor, a fibroblast growth factor 21 (FGF21), a niacin analogue, a leukotriene D4 (LTD4) receptor antagonist, an acetyl-CoA carboxylase (ACC) inhibitor, a ketohexokinase (KHK) inhibitor, an apoptosis signal-regulating kinase 1 (ASK1) inhibitor, an ileal bile acid transporter (IBAT) inhibitor, glycyrrhizin, Schisandra extract, ascorbic acid, glutathione, silymarin, lipoic acid, and d-alpha-tocopherol, ascorbic acid, glutathione, vitamin B-complex, glitazones/thiazolidinediones (e.g., troglitazone, rosiglitazone, pioglitazone), metformin, cysteamine, sulfonylureas, alpha-glucosidase inhibitors, meglitinides, vitamin E, tetrahydrolipstatin, milk thistle protein, anti-virals, and anti-oxidants.

In some embodiments, the one or more additional therapeutic agents include those useful, for example, for treating diabetic complications. Non-limiting examples include aldose reductase inhibitors (e.g., tolrestat, epalrestat, zopolrestat, fidarestat, CT-112, ranirestat, lidorestat), neurotrophic factor and increasing agents thereof (e.g., NGF, NT-3, BDNF, neurotrophic production/secretion promoting agents described in WO01/14372 (e.g., 4-(4-chlorophenyl)-2-(2-methyl-1-imidazolyl)-5-[3-(2-methylphenoxyl)propyl]oxazole), compounds described in WO2004/039365), PKC inhibitors (e.g., ruboxistaurin mesylate), AGE inhibitors (e.g., ALT946, N-phenacylthiazolium bromide (ALT766), EXO-226, pyridorin, pyridoxamine), serotonin and noradrenalin reuptake inhibitors (e.g., duloxetine), sodium channel inhibitors (e.g., lacosamide), active oxygen scavengers (e.g., thioctic acid), cerebral vasodilators (e.g., tiapuride, mexiletine), somatostatin receptor agonists (e.g., BIM23190), and apoptosis signal regulating kinase-1 (ASK-1) inhibitors.

In some embodiments, the one or more additional therapeutic agents include those useful, for example, for treating hyperlipidemia. Non-limiting examples include HMG-COA reductase inhibitors (e.g., pravastatin, simvastatin, lovastatin, atorvastatin, fluvastatin, rosuvastatin, pitavastatin or a salt thereof (e.g., sodium salt, calcium salt)), squalene synthase inhibitors (e.g., compounds described in WO97/10224, e.g., N-[[(3R,5S)-1-(3-acetoxy-2,2-dimethylpropyl)-7-chloro-5-(2,3-dimethoxyphenyl)-2-oxo-1,2,3,5-tetrahydro-4, 1-benzoxazepin-3-yl]acetyl]piperidin-4-acetic acid), fibrate compounds (e.g., bezafibrate, clofibrate, simfibrate, clinofibrate), anion exchange resin (e.g., colestyramine), nicotinic acid drugs (e.g., nicomol, niceritrol, niaspan), phytosterols (e.g., soysterol, gamma oryzanol (γ-oryzanol)), cholesterol absorption inhibitors (e.g., zechia), CETP inhibitors (e.g., dalcetrapib, anacetrapib) and ω-3 fatty acid preparations (e.g., ω-3-fatty acid ethyl esters 90).

In some embodiments, the one or more additional therapeutic agents include those useful, for example, as anti-hypertensive agents. Non-limiting examples include angiotensin converting enzyme inhibitors (e.g., captopril, enalapril, delapril), angiotensin II antagonists (e.g., candesartan cilexetil, candesartan, losartan, losartan potassium, eprosartan, valsartan, telmisartan, irbesartan, tasosartan, olmesartan, olmesartan medoxomil, azilsartan, azilsartan medoxomil), calcium antagonists (e.g., manidipine, nifedipine, amlodipine, efonidipine, nicardipine, cilnidipine) and β-blockers (e.g., metoprolol, atenolol, propranolol, carvedilol, pindolol).

In some embodiments, the one or more additional therapeutic agents include those useful, for example, as diuretics. Non-limiting examples include xanthine derivatives (e.g., theobromine sodium salicylate, theobromine calcium salicylate), thiazide preparations (e.g., ethiazide, cyclopenthiazide, trichloromethiazide, hydrochlorothiazide, hydroflumethiazide, benzylhydrochlorothiazide, penfluthiazide, polythiazide, methyclothiazide), antialdosterone preparations (e.g., spironolactone, triamterene), carbonic anhydrase inhibitors (e.g., acetazolamide) and chlorobenzenesulfonamide agents (e.g., chlortalidone, mefruside, indapamide).

In some embodiments, the one or more additional therapeutic agents include those useful, for example, as immunotherapeutic agents. Non-limiting examples include microbial or bacterial compounds (e.g., muramyl dipeptide derivative, picibanil), polysaccharides having immunoenhancing activity (e.g., lentinan, sizofiran, krestin), cytokines obtained by genetic engineering approaches (e.g., interferon, interleukin (IL) such as IL-1, IL-2, IL-12), and colony-stimulating factors (e.g., granulocyte colony-stimulating factor, erythropoietin).

In some embodiments, the one or more additional therapeutic agents include those useful, for example, as anti-thrombotic agents. Non-limiting examples include heparins (e.g., heparin sodium, heparin calcium, enoxaparin sodium, dalteparin sodium) warfarin (e.g., warfarin potassium); anti-thrombin drugs (e.g., aragatroban, dabigatran) FXa inhibitors (e.g., rivaroxaban, apixaban, edoxaban, betrixaban, YM150, compounds described in WO02/06234, WO2004/048363, WO2005/030740, WO2005/058823, and WO2005/113504) thrombolytic agents (e.g., urokinase, tisokinase, alteplase, nateplase, monteplase, pamiteplase), and platelet aggregation inhibitors (e.g., ticlopidine hydrochloride, clopidogrel, prasugrel, E5555, SHC530348, cilostazol, ethyl icosapentate, beraprost sodium, and sarpogrelate hydrochloride).

In some embodiments, the one or more additional therapeutic agents include those useful, for example, for treating osteoporosis. Non-limiting examples include alfacalcidol, calcitriol, elcatonin, calcitonin salmon, estriol, ipriflavone, pamidronate disodium, alendronate sodium hydrate, incadronate disodium, and risedronate disodium. Suitable examples of vitamins include vitamin B1 and vitamin B12. Suitable examples of erectile dysfunction drugs include apomorphine and sildenafil citrate. Suitable examples of therapeutic agents for urinary frequency or urinary incontinence include flavorxate hydrochloride, oxybutynin hydrochloride and propiverine hydrochloride. Suitable examples of therapeutic agents for dysuria include acetylcholine esterase inhibitors (e.g., distigmine). Suitable examples of anti-inflammatory agents include nonsteroidal anti-inflammatory drugs such as aspirin, acetaminophen, indomethacin.

Other exemplary additional therapeutic agents include agents that modulate hepatic glucose balance (e.g., fructose 1,6-bisphosphatase inhibitors, glycogen phosphorylase inhibitors, glycogen synthase kinase inhibitors, glucokinase activators), agents designed to treat the complications of prolonged hyperglycemia, such as aldose reductase inhibitors (e.g. epalrestat and ranirestat), agents used to treat complications related to micro-angiopathies, anti-dyslipidemia agents, such as HMG-CoA reductase inhibitors (statins, e.g. rosuvastatin), cholesterol-lowering agents, bile acid sequestrants (e.g., cholestyramine), cholesterol absorption inhibitors (e.g. plant sterols such as phytosterols), cholesteryl ester transfer protein (CETP) inhibitors, inhibitors of the ileal bile acid transport system (IBAT inhibitors), bile acid binding resins, nicotinic acid (niacin) and analogues thereof, anti-oxidants (e.g., probucol), omega-3 fatty acids, antihypertensive agents, including adrenergic receptor antagonists, such as beta blockers (e.g. atenolol), alpha blockers (e.g. doxazosin), and mixed alpha/beta blockers (e.g. labetalol), adrenergic receptor agonists, including alpha-2 agonists (e.g. clonidine), angiotensin converting enzyme (ACE) inhibitors (e.g. lisinopril), calcium channel blockers, such as dihydropridines (e.g. nifedipine), phenylalkylamines (e.g. verapamil), and benzothiazepines (e.g. diltiazem), angiotensin II receptor antagonists (e.g. candesartan), aldosterone receptor antagonists (e.g. eplerenone), centrally acting adrenergic drugs, such as central alpha agonists (e.g. clonidine), diuretic agents (e.g. furosemide), haemostasis modulators, including antithrombotics (e.g., activators of fibrinolysis), thrombin antagonists, factor VIIa inhibitors, anticoagulants (e.g., vitamin K antagonists such as warfarin), heparin and low molecular weight analogues thereof, factor Xa inhibitors, and direct thrombin inhibitors (e.g. argatroban), antiplatelet agents (e.g., cyclooxygenase inhibitors (e.g. aspirin)), adenosine diphosphate (ADP) receptor inhibitors (e.g. clopidogrel), phosphodiesterase inhibitors (e.g. cilostazol), glycoprotein IIB/IIA inhibitors (e.g. tirofiban), adenosine reuptake inhibitors (e.g. dipyridamole), noradrenergic agents (e.g. phentermine), serotonergic agents (e.g. sibutramine), diacyl glycerolacyltransferase (DGAT) inhibitors, feeding behavior modifying agents, pyruvate dehydrogenase kinase (PDK) modulators, serotonin receptor modulators, monoamine transmission-modulating agents, such as selective serotonin reuptake inhibitors (SSRI) (e.g. fluoxetine), noradrenaline reuptake inhibitors (NARI), noradrenaline-serotonin reuptake inhibitors (SNRI), and monoamine oxidase inhibitors (MAOI) (e.g. toloxatone and amiflamine), compounds described in W0007/013694, WO2007/018314, WO2008/093639 and WO2008/099794, GPR40 agonists (e.g., fasiglifam or a hydrate thereof, compounds described in WO2004/041266, WO2004/106276, WO2005/063729, WO2005/063725, WO2005/087710, WO2005/095338, WO2007/013689 and WO2008/001931), SGLT1 inhibitors, adiponectin or agonist thereof, IKK inhibitors (e.g., AS-2868), somatostatin receptor agonists, ACC2 inhibitors, cachexia-ameliorating agents, such as a cyclooxygenase inhibitors (e.g., indomethacin), progesterone derivatives (e.g., megestrol acetate), glucocorticoids (e.g., dexamethasone), metoclopramide agents, tetrahydrocannabinol agents, agents for improving fat metabolism (e.g., eicosapentaenoic acid), growth hormones, IGF-1, antibodies against a cachexia-inducing factor TNF-α, LIF, IL-6, and oncostatin M, metabolism-modifying proteins or peptides such as glucokinase (GK), glucokinase regulatory protein (GKRP), uncoupling proteins 2 and 3 (UCP2 and UCP3), peroxisome proliferator-activated receptor a (PPARα), MC4r agonists, insulin receptor agonist, PDE 5 inhibitors, glycation inhibitors (e.g., ALT-711), nerve regeneration-promoting drugs (e.g., Y-128, VX853, prosaptide), antidepressants (e.g., desipramine, amitriptyline, imipramine), antiepileptic drugs (e.g., lamotrigine, trileptal, keppra, zonegran, pregabalin, harkoseride, carbamazepine), antiarrhythmic drugs (e.g., mexiletine), acetylcholine receptor ligands (e.g., ABT-594), endothelin receptor antagonists (e.g., ABT-627), narcotic analgesics (e.g., morphine), α2 receptor agonists (e.g., clonidine), local analgesics (e.g., capsaicin), antianxiety drugs (e.g., benzothiazepine), phosphodiesterase inhibitors (e.g., sildenafil), dopamine receptor agonists (e.g., apomorphine), cytotoxic antibodies (e.g., T-cell receptor and IL-2 receptor-specific antibodies), B cell depleting therapies (e.g., anti-CD20 antibody (e.g., rituxan), i-BLyS antibody), drugs affecting T cell migration (e.g., anti-integrin alpha 4/beta 1 antibody (e.g., tysabri), drugs that act on immunophilins (e.g., cyclosporine, tacrolimus, sirolimus, rapamicin), interferons (e.g., IFN-β), immunomodulators (e.g., glatiramer), TNF-binding proteins (e.g., circulating receptors), immunosupressants (e.g., mycophenolate), and metaglidasen, AMG-131, balaglitazone, MBX-2044, rivoglitazone, aleglitazar, chiglitazar, lobeglitazone, PLX-204, PN-2034, GFT-505, THR-0921, exenatide, exendin-4, memantine, midazolam, ketoconazole, ethyl icosapentate, clonidine, azosemide, isosorbide, ethacrynic acid, piretanide, bumetanide, etoposide, piroxicam, NO donating agents (e.g., organonitrates), and NO promoting agents (e.g., phosphodiesterase inhibitors).

In some embodiments, the one or more additional therapeutic agents include those useful, for example, as anti-emetic agents. As used herein, an “anti-emetic” agent refers to any agent that counteracts (e.g., reduces or removes) nausea or emesis (vomiting). It is to be understood that when referring to a therapeutically effective amount of an anti-emetic agent, the amount administered is an amount needed to counteract (e.g., reduce or remove) nausea or emesis (vomiting). While not wishing to be bound by theory, it is believed that administering one or more anti-emetic agents in combination with the formula (I) compounds described herein may allow higher dosages of the formula (I) compounds to be administered, e.g., because the patient may be able to have a normal food intake and thereby respond faster to the treatment.

Non-limiting examples of anti-emetic agents include 5HT3-receptor antagonists (serotonin receptor antagonists), neuroleptics/anti-psychotics, antihistamines, anticholinergic agents, steroids (e.g., corticosteroids), NK1-receptor antagonists (e.g., Neurokinin 1 substance P receptor antagonists), antidopaminergic agents/dopamine receptor antagonists, benzodiazepines, cannabinoids.

For example, the antiemetic agent can be selected from the group consisting of; neuroleptics, antihistamines, anti-cholinergic agents, steroids, 5HT-3-receptor antagonists, NK1-receptor antagonists, anti-dopaminergic agents/dopamine receptor antagonists, benzodiazepines and non-psychoactive cannabinoids.

In some embodiments, the anti-emetic agent is a 5HT3-receptor antagonist (serotonin receptor antagonist). Non-limiting examples of 5HT3-receptor antagonists (serotonin receptor antagonists) include: granisetron (Kytril), dolasetron, ondansetron (Zofran), tropisetron, ramosetron, palonosetron, alosetron, azasetron, bemesetron, zatisetron, batanopirde, MDL-73147EF; Metoclopramide, N-3389 (endo-3,9-dimethyl-3,9-diazabicyclo[3,3,1]non-7-yl-1H-indazole-3-carboxamide dihydrochloride), Y-25130 hydrochloride, MDL 72222, Tropanyl-3,5-dimethylbenzoate, 3-(4-Allylpiperazin-1-yl)-2-quinoxalinecarbonitrile maleate, zacopride hydrochloride, and mirtazepine. Other non-limiting examples of 5HT3-receptor antagonists (serotonin receptor antagonists) include: cilansetron, clozapine, cyproheptadine, dazopride, hydroxyzine, lerisetron, metoclopramide, mianserin, olanzapine, palonosetron (+netupitant), quetiapine, qamosetron, ramosteron, ricasetron, risperidone, ziprasidone, and zatosetron.

In certain embodiments, the 5HT-3-receptor antagonist is granisetron, dolasetron, ondansetron hydrochloride, tropisetron, ramosetron, palonosetron, alosetron, bemesetron, zatisetron, batanopirde, MDL-73147EF, metoclopramide, N-3389, Y—25130 hydrochloride, MDL 72222, tropanyl-3,5-dimethylbenzoate 3-(4-AIIyI-piperazin-1-yl)-2-quinoxalinecarbonitrile maleate, zacopride hydrochloride and mirtazepine.

In certain embodiments, the 5HT-3-receptor antagonist is granisetron, dolasetron, ondansetron hydrochloride, tropisetron, ramosetron, palonosetron, alosetron, bemesetron, and zatisetron.

In certain embodiments, the 5HT-3-receptor antagonist is granisetron, dolasetron and ondansetron.

In certain embodiments, the 5HT-3-receptor antagonist is granisetron.

In certain embodiments, the 5HT-3-receptor antagonist is ondansetron.

In some embodiments, the anti-emetic agent is an antihistamine. Non-limiting examples of antihistamines include: piperazine derivatives (e.g., cyclizine, meclizine, and cinnarizine); promethazine; dimenhydrinate (Dramamine, Gravol); diphenhydramine; hydroxyzine; buclizine; and meclizine hydrochloride (Bonine, Antivert), doxylamine, and mirtazapine.

In some embodiments, the anti-emetic agent is an anticholinergic agent (inhibitors of the acetylcholine receptors). Non-limiting examples of anticholinergic agents include: atropine, scopolamine, glycopyrron, hyoscine, artane (trihexy-5 trihexyphenidyl hydrochloride), cogentin (benztropine mesylate), akineton (biperiden hydrochloride), disipal (norflex orphenadrine citrate), diphenhydramine, hydroxyzine, hyoscyamine, and kemadrin (procyclidine hydrochloride).

In some embodiments, the anti-emetic agent is a steroid (e.g., a corticosteroid). Non-limiting examples of steroids include: betamethasone, dexamethasone, methylprednisolone, Prednisone®, and trimethobenzamide (Tigan).

In some embodiments, the anti-emetic agent is an NK1-receptor antagonists (e.g., Neurokinin 1 substance P receptor antagonists). Non-limiting examples of NK1-receptor antagonists include: aprepitant, casopitant, ezlopitant, fosaprepitant, maropitant, netupitant, rolapitant, and vestipitant.

Other non-limiting examples of NK1-receptor antagonists include: MPC-4505, GW597599, MPC-4505, GR205171, L-759274, SR 140333, CP-96,345, BIIF 1149, NKP 608C, NKP 608A, CGP 60829, SR 140333 (Nolpitantium besilate/chloride), LY 303870 (Lanepitant), MDL-105172A, MDL-103896, MEN-11149, MEN-11467, DNK 333A, YM-49244, YM-44778, ZM-274773, MEN-10930, S-19752, Neuronorm, YM-35375, DA-5018, MK-869, L-754030, CJ-11974, L-758298, DNK-33A, 6b-1, CJ-11974 j. Benserazide and carbidopa k. TAK-637 [(aR,9R)-7-[3,5-bis(trifluoromethyl)benzyl]-8,9,10,11-tetrahydro-9-methyl-5-(4-methylphenyl)-7H-[1 ,4]diazocino[2,1-g][1,7]naphthyridine-6,13-dione], PD 154075, ([(2-benzofuran)-CH2OCO]—(R)-alpha-MeTrp-(S)—NHCH(CH₃) Ph), FK888, and (D-Pro4, D-Trp7,9,10, Phe11) SP4-11.

In some embodiments, the anti-emetic agent is an anti-dopaminergic agents/dopamine receptor antagonist (e.g., dopamine receptor antagonist, e.g., D2 or D3 antagonists). Non-limiting examples include phenothiazines (e.g., promethazine, chlorpromazine, prochlorperazine, perphenazine, hydroxyzine, thiethylperazine, metopimazine); benzamides (e.g., metoclopramide, domperidone), butyrophenones (e.g., haloperidol, droperidol); alizapride, bromopride, clebopride, domperidone, itopride, metoclopramide, trimethobenzamide, and amisulpride.

In some embodiments, the anti-emetic agent is a non-psychoactive cannabinoids (e.g., Cannabidiol (CBD), Cannabidiol dimethylheptyl (CBD-DMH), Tetra-hydro-cannabinol (THC), Cannabinoid agonists such as WIN 55-212 (a CB1 and CB2 receptor agonist), Dronabinol (Marinol®), and Nabilone (Cesamet)).

Other exemplary anti-emetic agents include: c-9280 (Merck); benzodiazepines (diazepam, midazolam, lorazepam); neuroleptics/anti-psychotics (e.g., dixyrazine, haloperidol, and Prochlorperazine (Compazine®)); cerium oxalate; propofol; sodium citrate; dextrose; fructose (Nauzene); orthophosphoric acid; fructose; glucose (Emetrol); bismuth subsalicylate (Pepto Bismol); ephedrine; vitamin B6; peppermint, lavender, and lemon essential oils; and ginger.

Still other exemplary anti-emetic agents include those disclosed in US 20120101089A1; U.S. Pat. No. 10,071,088 B2; U.S. Pat. No. 6,673,792 B1; U.S. Pat. No. 6,197,329 B1; U.S. Pat. No. 10,828,297 B2; U.S. Pat. No. 10,322,106 B2; U.S. Pat. No. 10,525,033 B2; WO 2009080351 A1; WO 2019203753 A2; WO 2002020001 A2; U.S. Pat. No. 8,119,697 B2; U.S. Pat. No. 5,039,528; US20090305964A1; and WO 2006/111169, each of which is incorporated by reference in its entirety.

In some embodiments, the additional therapeutic agent or regimen is administered to the patient prior to contacting with or administering the compounds and pharmaceutical compositions (e.g., about one hour prior, or about 6 hours prior, or about 12 hours prior, or about 24 hours prior, or about 48 hours prior, or about 1 week prior, or about 1 month prior).

In some embodiments, the additional therapeutic agent or regimen is administered to the patient at about the same time as contacting with or administering the compounds and pharmaceutical compositions. By way of example, the additional therapeutic agent or regimen and the compounds and pharmaceutical compositions are provided to the patient simultaneously in the same dosage form. As another example, the additional therapeutic agent or regimen and the compounds and pharmaceutical compositions are provided to the patient concurrently in separate dosage forms.

### Patient Selection

In some embodiments, the methods described herein further include the step of identifying a patient (e.g., a subject) in need of such treatment (e.g., by way of blood assay, body mass index, or other conventional method known in the art).

In some embodiments, the methods described herein further include the step of identifying a patient (e.g., patient) that has type 2 diabetes mellitus. In some embodiments, determining if the patient has type 2 diabetes mellitus includes performing an assay to determine the level of hemoglobin A1c (HbA1c), fasting plasma glucose, non-fasting plasma glucose, or any combination thereof. In some embodiments, the level ofHbAlc is about 6.5% to about 24.0%. In some embodiments, the level of HbA1c is greater than or about 6.5%. In some embodiments, the level of HbA1c is greater than or about 8.0%. In some embodiments, the level of HbA1c is greater than or about 10.0%. In some embodiments, the level of HbA1c is greater than or about 12.0%. In some embodiments, the level of HbA1c is greater than or about 14.0%. In some embodiments, the level of HbA1c is greater than or about 16.0%. In some embodiments, the level of HbA1c is greater than or about 18.0%. In some embodiments, the level of HbA1c is greater than or about 20.0%. In some embodiments, the level of HbA1c is greater than or about 22.0%. In some embodiments, the level of HbA1c is greater than or about 24.0%.

In some embodiments, the level of fasting plasma glucose is greater than or about 120 mg/dL to greater than or about 750 mg/dL. In some embodiments, the level of fasting plasma glucose is greater than or about 200 mg/dL to greater than or about 500 mg/dL. In some embodiments, the level of fasting plasma glucose is greater than or about 300 mg/dL to greater than or about 700 mg/dL.

In some embodiments, the level of non-fasting plasma glucose is greater than or about 190 mg/dL to greater than or about 750 mg/dL. In some embodiments, the level of non-fasting plasma glucose is greater than or about 250 mg/dL to greater than or about 450 mg/dL. In some embodiments, the level of non-fasting plasma glucose is greater than or about 400 mg/dL to greater than or about 700 mg/dL.

In some embodiments, determining if the patient has type 2 diabetes mellitus further includes determining the patient's BMI. In some embodiments, the BMI of the patient is greater than or about 22 kg/m2 to greater than or about 100 kg/m2. In some embodiments, the BMI of the patient is greater than or about 30 kg/m2 to greater than or about 90 kg/m2. In some embodiments, the BMI of the patient is greater than or about 40 kg/m2 to greater than or about 80 kg/m2. In some embodiments, the BMI of the patient is greater than or about 50 kg/m2 to greater than or about 70 kg/m2.

In some embodiments, additional factors (e.g. risk factors) used for determining if the patient has type 2 diabetes mellitus further includes age and ethnicity of the patient. In some embodiments, the patient's age is greater than or about 10 years. In some embodiments, the patient's age is greater than or about 15 years. In some embodiments, the patient's age is greater than or about 20 years. In some embodiments, the patient's age is greater than or about 25 years. In some embodiments, the patient's age is greater than or about 30 years. In some embodiments, the patient's age is greater than or about 35 years. In some embodiments, the patient's age is greater than or about 40 years. In some embodiments, the patient's age is greater than or about 42 years. In some embodiments, the patient's age is greater than or about 44 years. In some embodiments, the patient's age is greater than or about 46 years. In some embodiments, the patient's age is greater than or about 48 years. In some embodiments, the patient's age is greater than or about 50 years. In some embodiments, the patient's age is greater than or about 52 years. In some embodiments, the patient's age is greater than or about 54 years. In some embodiments, the patient's age is greater than or about 56 years. In some embodiments, the patient's age is greater than or about 58 years. In some embodiments, the patient's age is greater than or about 60 years. In some embodiments, the patient's age is greater than or about 62 years. In some embodiments, the patient's age is greater than or about 64 years. In some embodiments, the patient's age is greater than or about 66 years. In some embodiments, the patient's age is greater than or about 68 years. In some embodiments, the patient's age is greater than or about 70 years. In some embodiments, the patient's age is greater than or about 72 years. In some embodiments, the patient's age is greater than or about 74 years. In some embodiments, the patient's age is greater than or about 76 years. In some embodiments, the patient's age is greater than or about 78 years. In some embodiments, the patient's age is greater than or about 80 years. In some embodiments, the patient's age is greater than or about 85 years. In some embodiments, the patient's age is greater than or about 90 years. In some embodiments, the patient's age is greater than or about 95 years. In some embodiments, the ethnicity of the patient may be African American, American Indian or Alaska Native, Asian American, Hispanics or Latinos, or Native Hawaiian or Pacific Islander.

### Synthesis of the Compounds

The compounds of this disclosure can be prepared from readily available starting materials using, for example, the following general methods, and procedures. It will be appreciated that where certain process conditions (i.e., reaction temperatures, times, mole ratios of reactants, solvents, pressures, etc.) are given, other process conditions can also be used unless otherwise stated. Optimum reaction conditions may vary with the reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting certain functional groups are well known in the art. For example, numerous protecting groups are described in T. W. Greene and G. M. Wuts (1999) Protecting Groups in Organic Synthesis, 3rd Edition, Wiley, New York, and references cited therein.

Furthermore, the compounds of this disclosure may contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance CA USA), EMKA-Chemie Gmbh & Co. KG (Eching Germany), or Millipore Sigma (Burlington MA USA). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5^{th} Edition, 2001), and Larock's Comprehensive Organic Transformations (VCH Publishers Inc., 1989).

Scheme I illustrates a general method which can be employed for the synthesis of compounds described herein, where each wherein each of A, Ring B, L¹, L², R¹, R³, R⁴, and R⁵, are independently as defined herein, and R⁵⁰ is an alkyl or substituted alkyl.

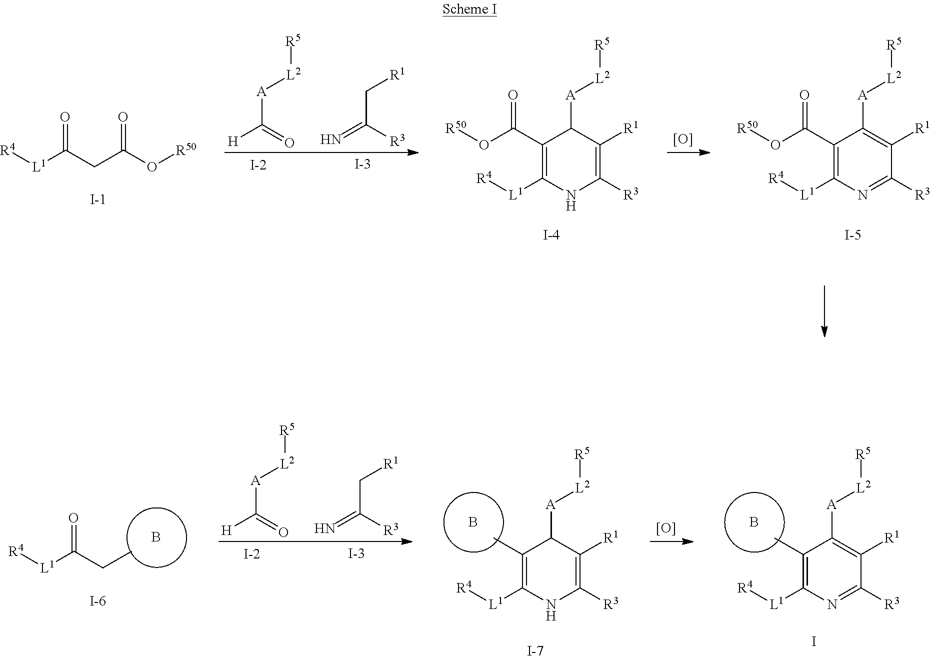

A Hantzsch style pyridine synthesis strategy can be used to synthesize the multi-substituted pyridine-based compound I-4 or compound I-7. As shown in Scheme I, coupling compound I-1 with compounds I-2 and I-3 provides I-4, and coupling compound I-6 with compounds I-2 and I-3 provides I-7. In some embodiments, the coupling reaction is performed under heated conditions in a suitable solvent (e.g., ethanol). Oxidation of I-4, such as by CAN or DDQ, provides compound I-5, which can then be further transformed into a heteroaryl using methods known in the art to provide compounds of Formula I. Oxidation of compound I-7 under similar reaction conditions provides compounds of Formula I.

For any compound shown in Scheme I, it should be understood that various derivatives can be provided by functional group interconversion at any step. In some embodiments, the various substituents of compounds I-1, I-2, I-3, I-4, I-5, I-6, or I-7 (e.g., A, Ring B, L, L², R¹, R³, R⁴, and R⁵) are as defined herein. However, derivatization of compounds I-1, I-2, I-3, I-4, I-5, I-6, or I-7 prior to reacting in any step, and/or further derivatization of the resulting reaction product, provides various compounds of Formula I. Appropriate starting materials and reagents can be purchased or prepared by methods known to one of skill in the art.

Upon reaction completion, compounds of Formula I can be recovered by conventional techniques such as neutralization, extraction, precipitation, chromatography, filtration and the like. In certain embodiments, when control of stereochemistry is desired, proper control of reaction conditions and selection of substituents for the reagents can at least partially dictate or preserve the formation of the various stereoisomers. Compounds I-1, I-2, and I-3 may be commercially obtained or synthesized de novo. For example, compound I-1 and I-6 compound I-1 may be prepared as shown in Scheme II or Scheme III.

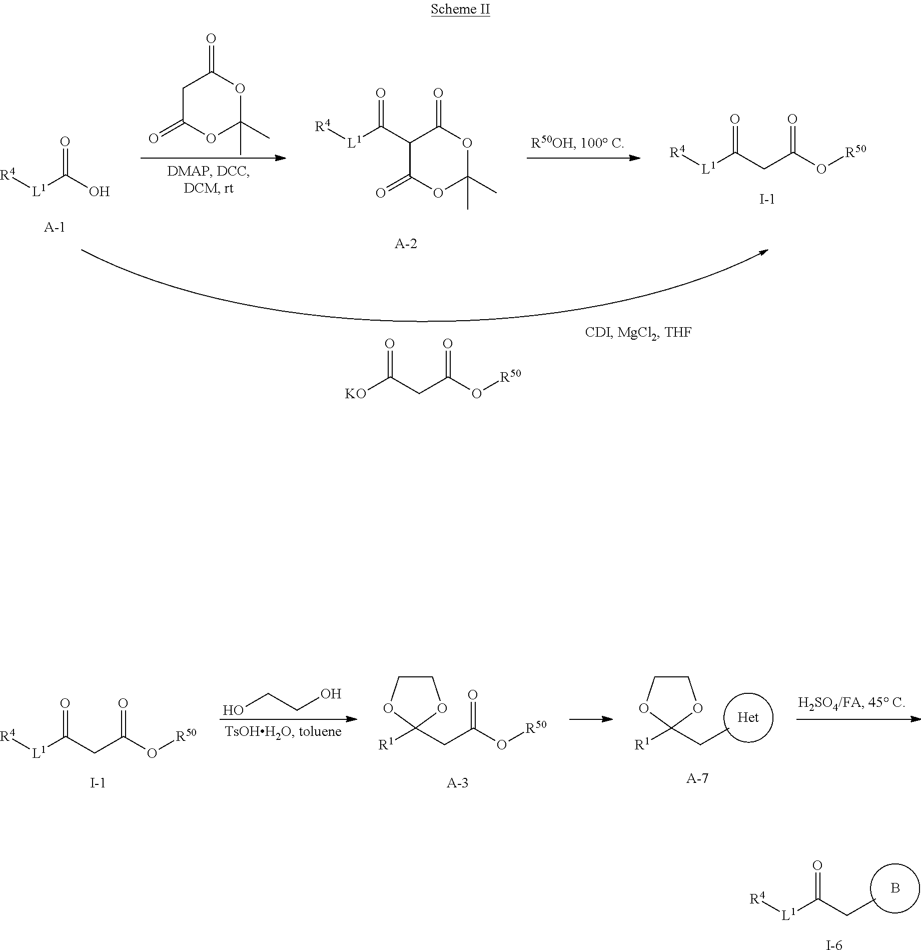

A-1 reacts with Meldrum's acid to give intermediate A-2, which undergoes decarboxylation to give 0-ketoacid compound I-1. Compound I-1 can be also directly synthesized by reacting A-1 with an alkyl potassium malonate in presence of CDI and MgCl₂.

Compound I-1 can could also react with ethane-1,2-diol to give intermediate A-3. Generally, the carboxylate group of A-3 can be transformed to various heteroaryl groups to give intermediate A-7, which undergoes deprotection by mixture of H₂SO₄ and formic acid to give compound I-6. For example, hydrolysis of the ester group of intermediate A-3 gives rise to a carboxylic acid, which can be reacted under conditions suitable to provide intermediate A-7 (e.g., reaction with acetohydrazide with the help of HATU and DIEA, which undergoes cyclization using 4-toluenesulfonyl chloride to give an oxadiazole). Deprotection of intermediate A-7 provides compound I-6.

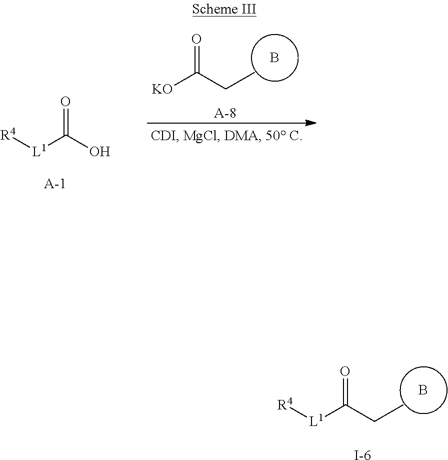

Carboxylic acid A-1 reacts with carboxylic acid potassium salt A-8 in presence of CDI and MgCl₂ gives compound I-6.

The following Scheme IV demonstrates a general scheme for the synthesis of indanyl amine I-7, which can be used to prepare compounds of Formula I where R⁵ is optionally substituted cycloalkyl.

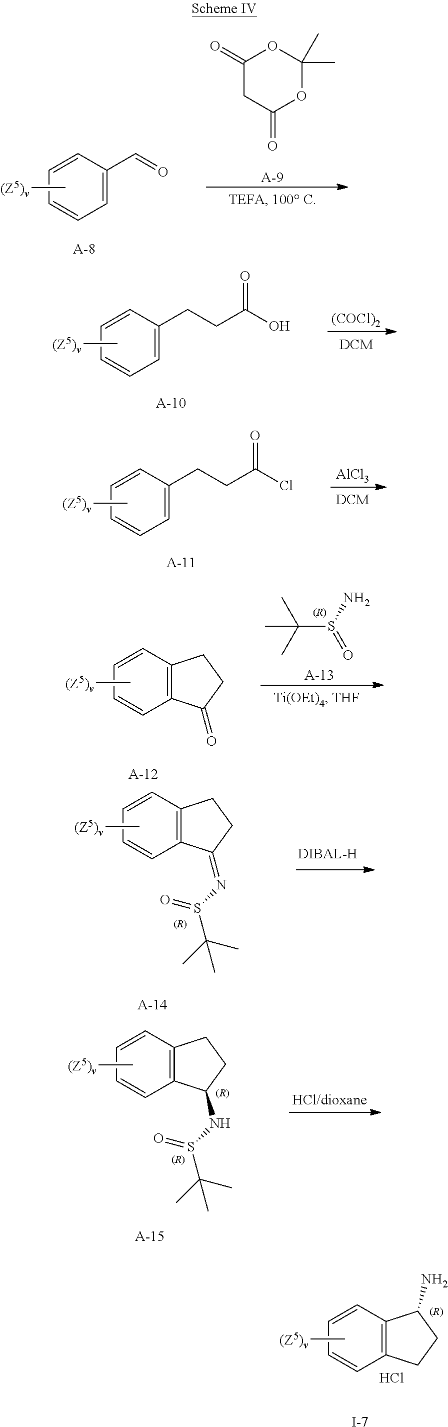

Aldehyde A-8 (where v is 0-5 and each Z⁵ is independently as defined herein) reacts with Meldrum's acid A-9 to give carboxylic acid A-10. It was reacted with oxalyl chloride to give its corresponding acyl chloride A-11 which undergoes an intra-molecular Friedel-Crafts acylation generating ketone A-12. It was then reacted with A-13 to give tert-butylsulfinamide A-14. Reduction of the sulfinamide using DIBAL-H gives A-15. Deprotection under acidic condition give rise to the indanyl amine intermediate I-7.

Upon each reaction completion, each of the intermediates or final compounds can be recovered, and optionally purified, by conventional techniques such as neutralization, extraction, precipitation, chromatography, filtration, and the like. Other modifications to arrive at compounds of this disclosure are within the skill of the art.

### General Synthesis

Typical embodiments of compounds described herein may be synthesized using the general reaction schemes described below. It will be apparent given the description herein that the general schemes may be altered by substitution of the starting materials with other materials having similar structures to result in products that are correspondingly different. Descriptions of syntheses follow to provide numerous examples of how the starting materials may vary to provide corresponding products. Given a desired product for which the substituent groups are defined, the necessary starting materials generally may be determined by inspection. Starting materials are typically obtained from commercial sources or synthesized using published methods. For synthesizing compounds which are embodiments described in the present disclosure, inspection of the structure of the compound to be synthesized will provide the identity of each substituent group. The identity of the final product will generally render apparent the identity of the necessary starting materials by a simple process of inspection, given the examples herein. In general, compounds described herein are typically stable and isolatable at room temperature and pressure.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

General information: All evaporations or concentrations were carried out in vacuo with a rotary evaporator. Analytical samples were dried in vacuo (1-5 mmHg) at rt. Thin layer chromatography (TLC) was performed on silica gel plates, spots were visualized by UV light (214 and 254 nm). Purification by column and flash chromatography was carried out using silica gel (100-200 mesh). Solvent systems were reported as mixtures by volume. NMR spectra were recorded on a Bruker 400 or Varian (400 MHz) spectrometer. ¹H chemical shifts are reported in 6 values in ppm with the deuterated solvent as the internal standard. Data are reported as follows: chemical shift, multiplicity (s=singlet, d=doublet, t=triplet, q=quartet, br=broad, m=multiplet), coupling constant (Hz), integration. LCMS spectra were obtained on SHIMADZU LC20-MS2020 or Agilent 1260 series 6125B mass spectrometer or Agilent 1200 series, 6110 or 6120 mass spectrometer with electrospray ionization and excepted as otherwise indicated.

### Example 1

### N—[(R)-1-indanyl]-5-{5-carbamoyl-2-[2-(p-fluorophenyl)ethyl]-6-isobutyl-3-(3-methyl-1,2,4-oxadiazol-5-yl)-4-pyridyl}-2-thenamide (Compound 101)

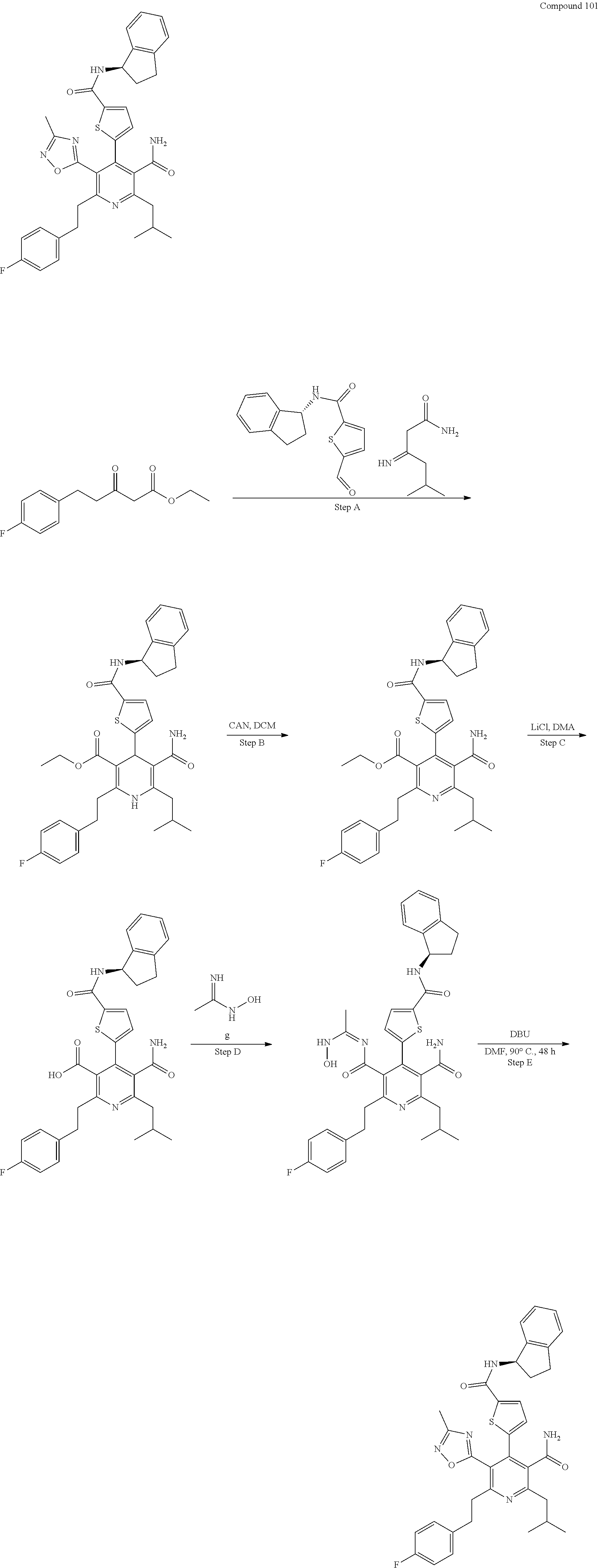

### Step A ethyl 5-carbamoyl-2-[2-(4-fluorophenyl)ethyl]-4-[5-({[(1R)-2,3-dihydro-1H-indenyl]amino}carbonyl)thiophen-2-yl]-6-(2-methylpropyl)-1, 4-dihydropyridine-3-carboxylate

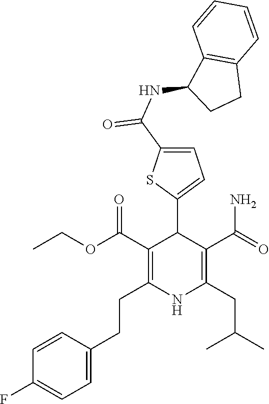 [SMILES: CCOC(=O)C1=C(CCc2ccc(F)cc2)NC(CC(C)C)=C(C(N)=O)C1c1ccc(C(=O)N[C@@H]2CCc3ccccc32)s1]

A solution of ethyl 5-(4-fluorophenyl)-3-oxopentanoate (418.88 mg, 1.758 mmol) in EtOH (10 mL) were added N-[(1R)-2,3-dihydro-1H-indenyl]-5-formylthiophene-2-carboxamide (477.02 mg, 1.758 mmol) and 3-azanylidene-5-methylhexanamide (300 mg, 2.110 mmol), and the reaction mixture was stirred at 100° C. for 18 hrs. The reaction mixture was concentrated to give ethyl 5-carbamoyl-2-[2-(4-fluorophenyl)ethyl]-4-[5-({[(1R)-2,3-dihydro-1H-indenyl]amino} carbonyl)thiophen-2-yl]-6-(2-methylpropyl)-1,4-dihydropyridine-3-carboxylate (1.1 g, crude). LC-MS: m/z 615.8 (M+H)⁺.

### Step B ethyl 5-carbamoyl-2-[2-(4-fluorophenyl)ethyl]-4-[5-({[(1R)-2,3-dihydro-1H-indenyl]amino}carbonyl)thiophen-2-yl]-6-(2-methylpropyl)pyridine-3-carboxylate

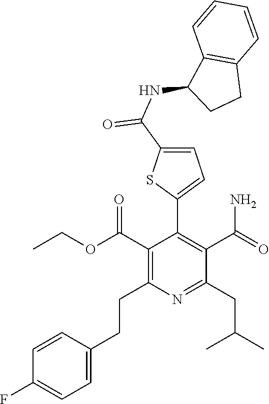 [SMILES: CCOC(=O)c1c(CCc2ccc(F)cc2)nc(CC(C)C)c(C(N)=O)c1-c1ccc(C(=O)N[C@@H]2CCc3ccccc32)s1]

To a solution of ethyl 5-carbamoyl-2-[2-(4-fluorophenyl)ethyl]-4-[5-({[(1R)-2,3-dihydro-1H-indenyl]amino}carbonyl)thiophen-2-yl]-6-(2-methylpropyl)-1,4-dihydropyridine-3-carboxylate (1.1 g, 1.786 mmol) in DCM (15 mL) were added ceric ammonium nitrate (1.47 g, 2.680 mmol), and the reaction mixture was stirred at 50° C. for 2 hrs. The reaction mixture was diluted with EtOAc and water. The organic layer was separated, washed with further water, and concentrated in vacuo. The residue was purified by silica gel column chromatography, eluting with ethyl acetate in petroleum ether. The organic layer was collected, concentrated in vacuo to give ethyl 5-carbamoyl-2-[2-(4-fluorophenyl)ethyl]-4-[5-({[(1R)-2,3-dihydro-1H-indenyl]amino}carbonyl)thiophen-2-yl]-6-(2-methylpropyl)pyridine-3-carboxylate (420 mg, 38.31%). LC-MS: m/z 614.0 (M+H)⁺.

### Step C 5-carbamoyl-2-[2-(4-fluorophenyl)ethyl]-4-[5-({[(1R)-2,3-dihydro-1H-indenyl]amino}carbonyl)thiophen-2-yl]-6-(2-methylpropyl)pyridine-3-carboxylic acid

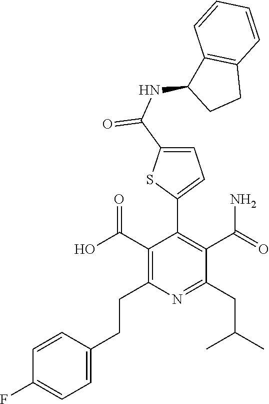 [SMILES: CC(C)Cc1nc(CCc2ccc(F)cc2)c(C(=O)O)c(-c2ccc(C(=O)N[C@@H]3CCc4ccccc43)s2)c1C(N)=O]

To a solution of ethyl 5-carbamoyl-2-[2-(4-fluorophenyl)ethyl]-4-[5-({[(1R)-2,3-dihydro-1H-indenyl]amino}carbonyl)thiophen-2-yl]-6-(2-methylpropyl)pyridine-3-carboxylate (400 mg, 0.652 mmol) in DMA (1 mL) were added LiCl (138.13 mg, 3.259 mmol), The reaction mixture was stirred at 150° C. for 5 hrs under M.W. The reaction mixture was purified by column chromatography, eluting with FA/H₂O/ACN. The organic layer was collected, concentrated in vacuo, and dried to give 5-carbamoyl-2-[2-(4-fluorophenyl)ethyl]-4-[5-({[(1R)-2,3-dihydro-1H-indenyl]amino}carbonyl)thiophen-2-yl]-6-(2-methylpropyl)pyridine-3-carboxylic acid (100 mg, 26.20%). LC-MS: m/z 586.3 (M+H)⁺.

### Step D 6-[2-(4-fluorophenyl)ethyl]-4-[5-({[(1R)-2,3-dihydro-1H-indenyl]amino}carbonyl) thiophen-2-yl]-5-({[(1Z)-1-(hydroxyamino)ethylidene]amino}carbonyl)-2-(2-methylpropyl)pyridine-3-carboxamide

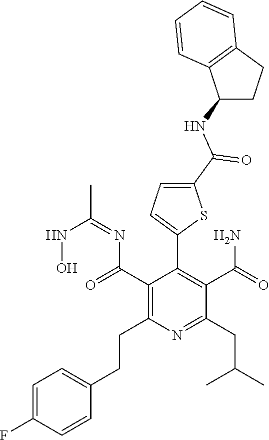 [SMILES: C/C(=N/C(=O)c1c(CCc2ccc(F)cc2)nc(CC(C)C)c(C(N)=O)c1-c1ccc(C(=O)N[C@@H]2CCc3ccccc32)s1)NO]

To a solution of 5-carbamoyl-2-[2-(4-fluorophenyl)ethyl]-4-[5-({[(1R)-2,3-dihydro-1H-indenyl]amino} carbonyl)thiophen-2-yl]-6-(2-methylpropyl)pyridine-3-carboxylic acid (50 mg, 0.085 mmol) in DMF (1 mL) were added N-(1-azanylideneethyl)hydroxylamine (7.59 mg, 0.102 mmol), DIEA (33.10 mg, 0.256 mmol), and PyBOP (53.31 mg, 0.102 mmol), and the reaction mixture was stirred at room temperature for 1 hr. The reaction mixture was diluted with EtOAc (50 mL) and water (20 mL). The organic layer was separated, washed with further water (20 mL*2) and saturated NaCl aq. (20 mL). The organic layer was separated, dried with Na₂SO₄ and then filtered. The organic layer was collected, concentrated in vacuo. The residue was purified by silica gel column chromatography, eluting with PE:EtOAc=1:1. The organic layer was collected, concentrated in vacuo, and dried to give 6-[2-(4-fluorophenyl)ethyl]-4-[5-({[(1R)-2,3-dihydro-1H-indenyl]amino}carbonyl)thiophen-2-yl]-5-({[(1Z)-1-(hydroxyamino)ethylidene]amino} carbonyl)-2-(2-methylpropyl)pyridine-3-carboxamide (50 mg, 91.26%). LC-MS: m/z 642.2 (M+H)⁺.

### Step E N—[(R)-1-indanyl]-5-{5-carbamoyl-2-[2-(p-fluorophenyl)ethyl]-6-isobutyl-3-(3-methyl-1,2,4-oxadiazol-5-yl)-4-pyridyl}-2-thenamide (Compound 101)

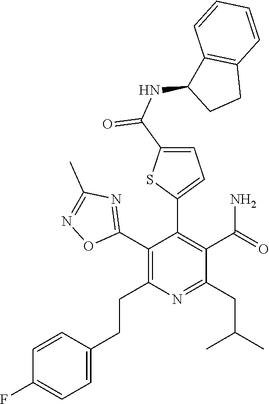 [SMILES: Cc1noc(-c2c(CCc3ccc(F)cc3)nc(CC(C)C)c(C(N)=O)c2-c2ccc(C(=O)N[C@@H]3CCc4ccccc43)s2)n1]

To a solution of 6-[2-(4-fluorophenyl)ethyl]-4-[5-({[(1R)-2,3-dihydro-1H-indenyl]amino}carbonyl)thiophen-2-yl]-5-({[(1Z)-1-(hydroxyamino)ethylidene]amino}carbonyl)-2-(2-methylpropyl)pyridine-3-carboxamide (40 mg, 0.062 mmol) in DMF (2 mL) was added DBU (37.96 mg, 0.249 mmol), and the reaction mixture was stirred at 90° C. for 18 hrs. The reaction mixture was filtered and purified by Prep-HPLC (FA) to give N—[(R)-1-indanyl]-5-{5-carbamoyl-2-[2-(p-fluorophenyl)ethyl]-6-isobutyl-3-(3-methyl-1,2,4-oxadiazol-5-yl)-4-pyridyl}-2-thenamide (15.53 mg, 0.025 mmol, 39.95%).

¹H NMR (400 MHz, DMSO-d₆) δ 8.87 (d, J=8.4 Hz, 1H), 7.91 (s, 1H), 7.68 (d, J=3.6 Hz, 1H), 7.60-7.64 (m, 1H), 7.16-7.30 (m, 4H), 7.08-7.15 (m, 2H), 7.03-7.06 (m, 2H), 6.94 (d, J=3.6 Hz, 1H), 5.43-5.51 (m, 1H), 2.93-3.08 (m, 5H), 2.79-2.89 (m, 1H), 2.73 (d, J=3.6 Hz, 2H), 2.43-2.48 (m, 1H), 2.38 (s, 3H), 2.25-2.34 (m, 1H), 1.88-1.99 (m, 1H), 0.94 (d, J=6.4 Hz, 6H). ¹⁹F NMR (377 MHz, DMSO-d₆) δ −117.22. LC-MS: m/z 624.3 (M+H)⁺.

### N-(3,4-difluorophenyl)methyl-5-{5-carbamoyl-2-[2-(p-fluorophenyl)ethyl]-6-isobutyl-3-(3-methyl-1,2,4-oxadiazol-5-yl)-4-pyridyl}-2-thenamide (Compound 102)

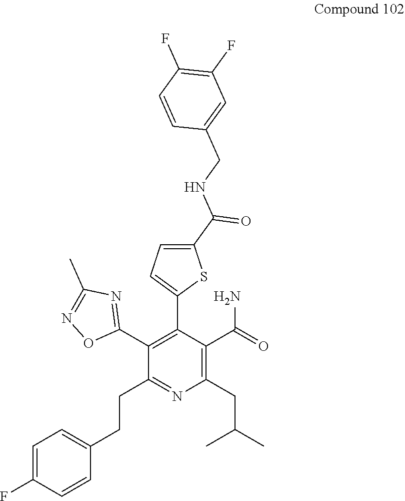 [SMILES: Cc1noc(-c2c(CCc3ccc(F)cc3)nc(CC(C)C)c(C(N)=O)c2-c2ccc(C(=O)NCc3ccc(F)c(F)c3)s2)n1]

Compound 102 was synthesized using a similar procedure described in the Example 1 above by using the appropriate materials.

¹H NMR (400 MHz, DMSO-d₆) δ 9.12 (t, J=6.0 Hz, 1H), 7.89 (s, 1H), 7.64 (d, J=3.6 Hz, 1H), 7.60 (s, 1H), 7.30-7.44 (m, 2H), 7.09-7.18 (m, 3H), 7.05 (t, J=8.8 Hz, 2H), 6.97 (d, J=4.0 Hz, 1H), 4.40 (d, J=5.6 Hz, 2H), 2.94-2.99 (m, 4H), 2.73 (d, J=6.8 Hz, 2H), 2.36 (s, 3H), 2.24-2.35 (m, 1H), 0.93 (d, J=6.8 Hz, 6H). ¹⁹F NMR (377 MHz, DMSO-d₆) δ −117.24, −138.84, −141.32. LC-MS: m/z 634.2 (M+H)⁺.

### N-(3,4-difluorophenyl)methyl-5-{5-carbamoyl-2-[2-(p-fluorophenyl)ethyl]-6-isobutyl-3-(3-isopropyl-1,2,4-oxadiazol-5-yl)-4-pyridyl}-2-thenamide (Compound 103)

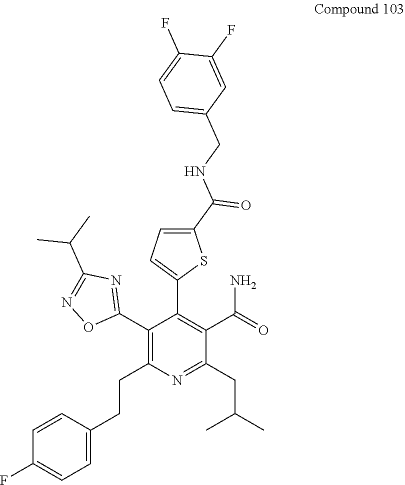 [SMILES: CC(C)Cc1nc(CCc2ccc(F)cc2)c(-c2nc(C(C)C)no2)c(-c2ccc(C(=O)NCc3ccc(F)c(F)c3)s2)c1C(N)=O]

Compound 103 was synthesized using a similar procedure described in the Example 1 above by using the appropriate materials.

¹H NMR (400 MHz, DMSO-d₆) δ 9.12 (t, J=6.0 Hz, 1H), 7.90 (s, 1H), 7.58-7.67 (m, 2H), 7.19-7.45 (m, 2H), 7.00-7.07 (m, 5H), 6.97 (d, J=3.6 Hz, 1H), 4.40 (d, J=6.0 Hz, 2H), 3.01-3.12 (m, 1H), 2.91-2.98 (m, 4H), 2.74 (d, J=7.2 Hz, 2H), 2.23-2.36 (m, 1H), 1.18 (d, J=6.8 Hz, 6H), 0.94 (d, J=6.8 Hz, 6H). ¹⁹F NMR (377 MHz, DMSO-d₆) δ −117.18, −138.88, −141.36. LC-MS: m/z 662.2 (M+H)⁺.

### Example 2

### 4-(5-{1-[(3,4-difluorophenyl)methyl]-1H-1,2,3-triazol-4-yl}-2-thienyl)-6-[2-(p-fluorophenyl)ethyl]-2-isobutyl-5-(3-methyl-1,2,4-oxadiazol-5-yl)nicotinamide (Compound 104)

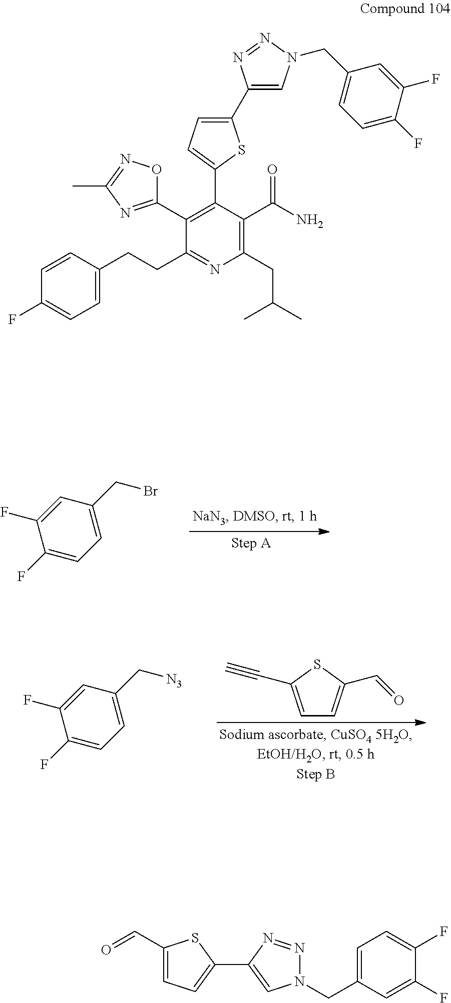

### Step A 4-(azidomethyl)-1,2-difluorobenzene

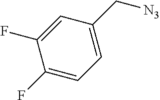 [SMILES: [N-]=[N+]=NCc1ccc(F)c(F)c1]

To a mixture of 4-(bromomethyl)-1,2-difluorobenzene (500 mg, 3.873 mmol) in DMSO (10 mL) was added NaN₃ (669.25 mg, 5.809 mmol). The mixture was stirred at room temperature for 1 hr. The reaction mixture was quenched with aq. NaHCO₃ (25 mL) and extracted with EtOAc (25 mL×3). The organic layer was combined and washed with brine (25 mL×2), dried over Na₂SO₄, filtered and concentrated in vacuum to give 4-(azidomethyl)-1,2-difluorobenzene (370 mg, crude).

### Step B 5-(1-(3,4-difluorobenzyl)-1H-1,2,3-triazol-4-yl)thiophene-2-carbaldehyde

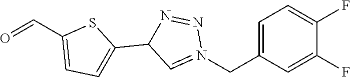

A mixture of 4-(azidomethyl)-1,2-difluorobenzene (370 mg, crude), 5-ethynylthiophene-2-carbaldehyde (307.26 mg, 2.26 mmol) in EtOH (4.0 mL) and H₂O (4.0 mL) was added cupric sulfate pentakis(oxidane) (28.17 mg, 0.113 mmol) and Sodium ascorbate (44.70 mg, 0.226 mmol) at room temperature. The mixture was stirred at room temperature for 1 hr. The reaction mixture was quenched with H₂O (20 mL) and extracted with EtOAc (20 mL×3). The organic layer was combined and washed with brine (20 mL×2), dried over Na₂SO₄, filtered and concentrated in vacuum. The residue was purified by column chromatography (PE/EtOAc=3/1) to give 5-(1-(3,4-difluorobenzyl)-1H-1,2,3-triazol-4-yl)thiophene-2-carbaldehyde (148 mg, 22.19%). LC-MS: m/z 306.1 (M+H)⁺.

### 4-(5-{1-[(3,4-difluorophenyl)methyl]-1H-1,2,3-triazol-4-yl}-2-thienyl)-6-[2-(p-fluorophenyl)ethyl]-2-isobutyl-5-(3-methyl-1,2,4-oxadiazol-5-yl)nicotinamide (Compound 104)

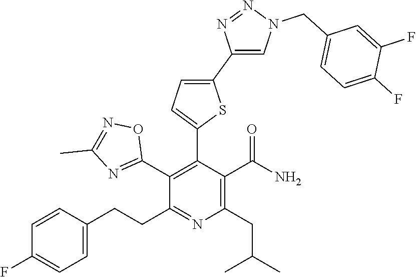 [SMILES: Cc1noc(-c2c(CCc3ccc(F)cc3)nc(CC(C)C)c(C(N)=O)c2-c2ccc(-c3cn(Cc4ccc(F)c(F)c4)nn3)s2)n1]

Compound 104 was then synthesized using a similar procedure described in the Example 1 above by using the appropriate materials.

¹H NMR (400 MHz, DMSO-d₆) δ 8.57 (s, 1H), 7.91 (s, 1H), 7.61 (s, 1H), 7.43-7.53 (m, 2H), 7.30 (d, J=3.6 Hz, 1H), 7.03-7.23 (m, 5H), 6.95 (d, J=4.0 Hz, 1H), 5.64 (s, 2H), 2.91-2.97 (m, 4H), 2.73 (d, J=7.2 Hz, 2H), 2.26-2.36 (m, 4H), 0.94 (d, J=6.8 Hz, 6H). ¹⁹F NMR (377 MHz, DMSO-d₆) δ −117.24, 137.87, 139.14. LC-MS: m/z 658.0 (M+H)⁺.

### Example 3

### N-(3,4-difluorophenyl)methyl-5-{5-carbamoyl-2-[2-(p-fluorophenyl)ethyl]-6-isobutyl-3-[3-(trifluoromethyl)-1,2,4-oxadiazol-5-yl]-4-pyridyl}-2-thenamide (Compound 105)

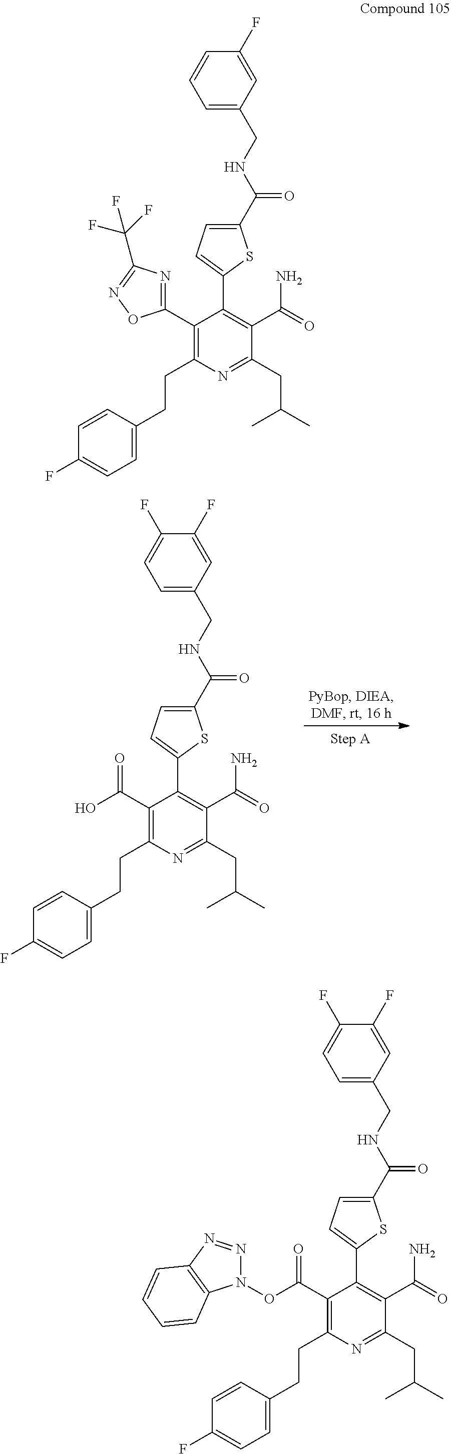

### Step A 1H-benzo[d][1,2,3]triazol-1-yl 5-carbamoyl-4-(5-((3,4-difluorobenzyl)carbamoyl)thiophen-2-yl)-2-(4-fluorophenethyl)-6-isobutylnicotinate

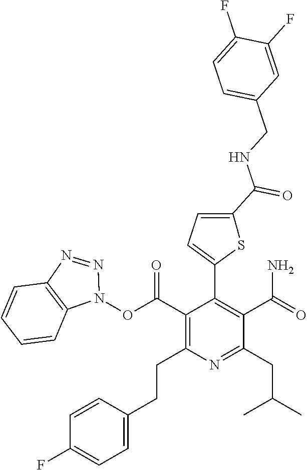 [SMILES: CC(C)Cc1nc(CCc2ccc(F)cc2)c(C(=O)On2nnc3ccccc32)c(-c2ccc(C(=O)NCc3ccc(F)c(F)c3)s2)c1C(N)=O]

To a solution of 5-carbamoyl-4-(5-((3,4-difluorobenzyl)carbamoyl)thiophen-2-yl)-2-(4-fluorophenethyl)-6-isobutylnicotinic acid (50 mg, 0.084 mmol) in DMF (1.5 mL) were added DIEA (33 mg, 0.252 mmol) and PyBOP (66 mg, 0.126 mmol). The reaction mixture was stirred at room temperature for 16 hrs. The reaction mixture was diluted with EtOAc (15 mL*3) and water (10 mL). The organic layer was separated, washed with further saturated NaCl solution (10 mL), and concentrated in vacuo. The residue was purified by silica gel column chromatography eluting with 10% methanol in dichloroform and concentrated to give 1H-benzo[d][1,2,3]triazol-1-yl 5-carbamoyl-4-(5-((3,4-difluorobenzyl)carbamoyl)thiophen-2-yl)-2-(4-fluorophenethyl)-6-isobutylnicotinate (45 mg, 75.2%). LC-MS: m/z 713.2 (M+H)⁺.

### N-(3,4-difluorophenyl)methyl-5-{5-carbamoyl-2-[2-(p-fluorophenyl)ethyl]-6-isobutyl-3-[3-(trifluoromethyl)-1,2,4-oxadiazol-5-yl]-4-pyridyl}-2-thenamide (Compound 105)

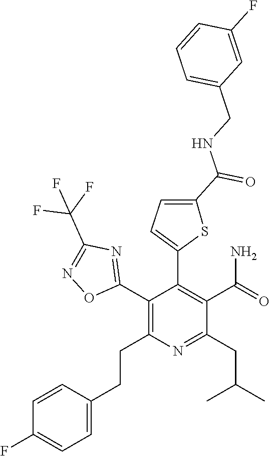 [SMILES: CC(C)Cc1nc(CCc2ccc(F)cc2)c(-c2nc(C(F)(F)F)no2)c(-c2ccc(C(=O)NCc3cccc(F)c3)s2)c1C(N)=O]

Compound 105 was then synthesized using a similar procedure described in the Example 1 above by using the appropriate materials.

¹H NMR (400 MHz, DMSO-d₆) δ 9.15 (t, J=6.4 Hz, 1H), 7.93 (s, 1H), 7.66 (d, J=3.6 Hz, 2H), 7.28-7.44 (m, 2H), 7.11-7.17 (m, 3H), 6.98-7.06 (m, 3H), 4.40 (d, J=6.0 Hz, 2H), 2.96-3.14 (m, 4H), 2.76 (d, J=9.2 Hz, 2H), 2.25-2.39 (m, 1H), 0.95 (d, J=6.8 Hz, 6H). ¹⁹F NMR (377 MHz, DMSO-d₆) δ −65.33, −117.16, 138.90, 141.33. LC-MS: m/z 688.1 (M+H)⁺.

### Example 4

### N—[(R)-1-indanyl]-5-{5-carbamoyl-2-[2-(p-fluorophenyl)ethyl]-6-isobutyl-3-(5-methyl-1,3,4-oxadiazol-2-yl)-4-pyridyl}-2-thenamide (Compound 106)

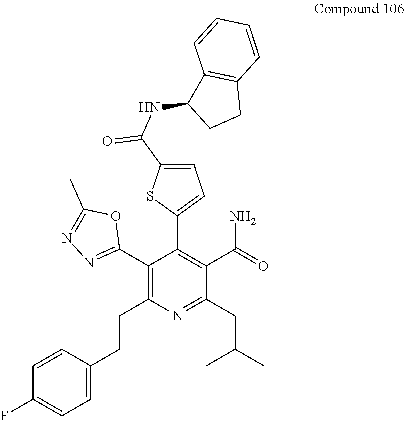

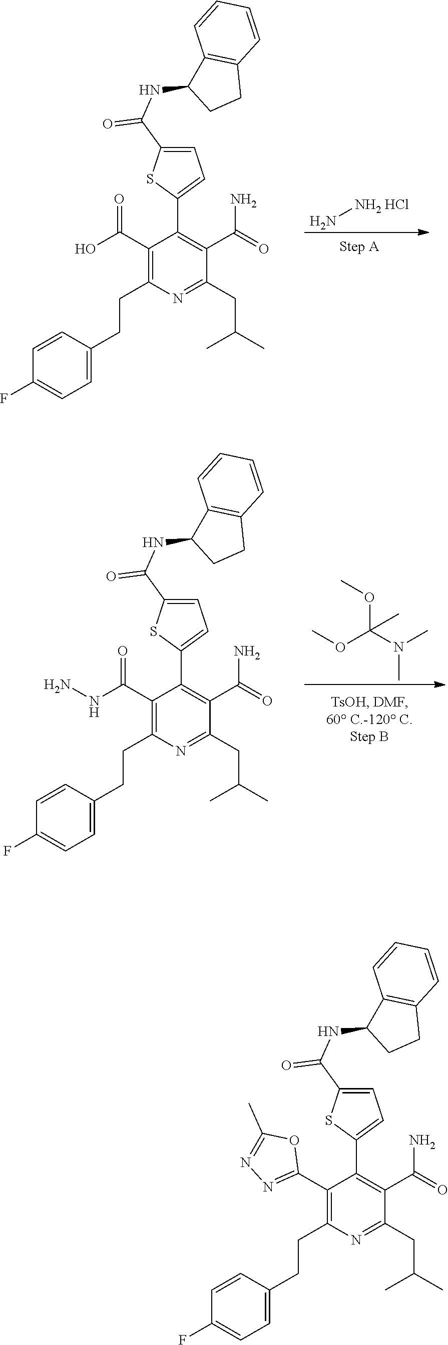

### Step A 5-(diazanylcarbonyl)-6-[2-(4-fluorophenyl)ethyl]-4-[5-({[(1R)-2,3-dihydro-1H-indenyl]amino} carbonyl)thiophen-2-yl]-2-(2-methylpropyl)pyridine-3-carboxamide

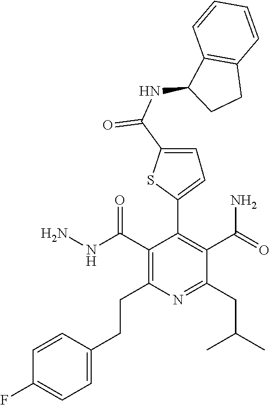 [SMILES: CC(C)Cc1nc(CCc2ccc(F)cc2)c(C(=O)NN)c(-c2ccc(C(=O)N[C@@H]3CCc4ccccc43)s2)c1C(N)=O]

To a solution of 5-carbamoyl-2-[2-(4-fluorophenyl)ethyl]-4-[5-({[(1R)-2,3-dihydro-1H-indenyl]amino}carbonyl)thiophen-2-yl]-6-(2-methylpropyl)pyridine-3-carboxylic acid (200 mg, 0.341 mmol) in DMF (2 mL) were added hydrazine hydrochloride (0.011 mL, 0.341 mmol), DIEA (132.41 mg, 1.024 mmol), and PyBOP (213.25 mg, 0.410 mmol), and the reaction mixture was stirred at room temperature for 1 hr. The reaction mixture was diluted with EtOAC (50 mL) and water (20 mL). The organic layer was separated, washed with further water (20 mL*2) and saturated NaCl aq. (20 mL). The organic layer was separated, dried with Na₂SO₄ and then filtered. The organic layer was collected, concentrated in vacuo. The residue was purified by silica gel column chromatography, eluting with PE:EtOAc=1:1. The organic layer was collected, concentrated in vacuo, and dried to give 5-(diazanylcarbonyl)-6-[2-(4-fluorophenyl)ethyl]-4-[5-({[(1R)-2,3-dihydro-1H-indenyl]amino}carbonyl)thiophen-2-yl]-2-(2-methylpropyl)pyridine-3-carboxamide (250 mg, crude). LC-MS: m/z 599.8 (M+H)⁺.

### Step B N—[(R)-1-indanyl]-5-{5-carbamoyl-2-[2-(p-fluorophenyl)ethyl]-6-isobutyl-3-(5-methyl-1,3,4-oxadiazol-2-yl)-4-pyridyl}-2-thenamide (Compound 106)

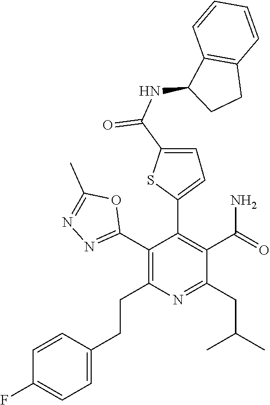 [SMILES: Cc1nnc(-c2c(CCc3ccc(F)cc3)nc(CC(C)C)c(C(N)=O)c2-c2ccc(C(=O)N[C@@H]3CCc4ccccc43)s2)o1]

To a solution of 5-(diazanylcarbonyl)-6-[2-(4-fluorophenyl)ethyl]-4-[5-({[(1R)-2,3-dihydro-1H-indenyl]amino} carbonyl)thiophen-2-yl]-2-(2-methylpropyl)pyridine-3-carboxamide (200 mg, 0.333 mmol) in DMF (2 mL) were added 1-(dimethylamino)-1,1-dimethoxyethane (66.62 mg, 0.500 mmol), and the reaction mixture was stirred at 60° C. for 3 hrs. The reaction mixture was concentrated, and the residue was solved in Toluene (4 mL). TsOH (6.37 mg, 0.033 mmol) was added into the reaction mixture and the reaction mixture was stirred at 120° C. for another 2 hrs. The reaction mixture was concentrated and the residue was purified by Prep-HPLC (FA) to give N—[(R)-1-indanyl]-5-{5-carbamoyl-2-[2-(p-fluorophenyl)ethyl]-6-isobutyl-3-(5-methyl-1,3,4-oxadiazol-2-yl)-4-pyridyl}-2-thenamide (27.97 mg, 0.045 mmol, 13.45%).

¹H NMR (400 MHz, DMSO-d₆) δ 8.84 (d, J=8.4 Hz, 1H), 7.88 (br s, 1H), 7.67 (d, J=4.0 Hz, 1H), 7.58 (br s, 1H), 7.16-7.30 (m, 4H), 7.02-7.14 (m, 4H), 6.94 (d, J=4.0 Hz, 1H), 5.42-5.51 (m, 1H), 2.93-3.03 (m, 5H), 2.79-2.89 (m, 1H), 2.73 (d, J=7.2 Hz, 2H), 2.42-2.46 (m, 1H), 2.40 (s, 3H), 2.25-2.35 (m, 1H), 1.88-1.99 (m, 1H), 0.94 (d, J=6.4 Hz, 6H). ¹⁹F NMR (377 MHz, DMSO-d₆) δ −117.28. LC-MS: m/z 624.4 (M+H)⁺.

### N-(3,4-difluorophenyl)methyl-5-{5-carbamoyl-2-[2-(p-fluorophenyl)ethyl]-6-isobutyl-3-(5-methyl-1,3,4-oxadiazol-2-yl)-4-pyridyl}-2-thenamide (Compound 107)

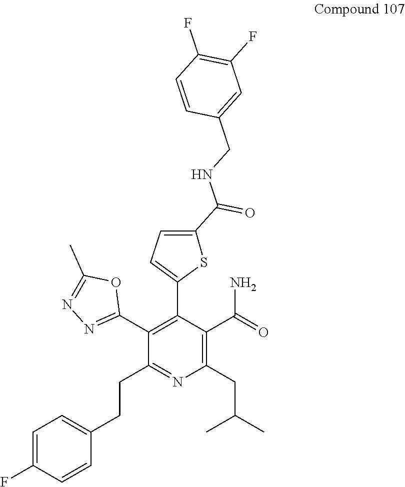 [SMILES: Cc1nnc(-c2c(CCc3ccc(F)cc3)nc(CC(C)C)c(C(N)=O)c2-c2ccc(C(=O)NCc3ccc(F)c(F)c3)s2)o1]

Compound 107 was synthesized using a similar procedure described in the Example 4 above by using the appropriate materials.

¹H NMR (400 MHz, DMSO-d₆) δ 9.12 (t, J=6.0 Hz, 1H), 7.89 (s, 1H), 7.64 (d, J=3.6 Hz, 1H), 7.59 (s, 1H), 7.30-7.45 (m, 2H), 7.01-7.18 (m, 5H), 6.97 (d, J=4.0 Hz, 1H), 4.40 (d, J=6.0 Hz, 2H), 2.94-3.01 (m, 4H), 2.73 (d, J=6.8 Hz, 2H), 2.38 (s, 3H), 2.25-2.35 (m, 1H), 0.93 (d, J=6.8 Hz, 6H). ¹⁹F NMR (377 MHz, DMSO-d₆) δ −117.26, −138.84, −141.33. LC-MS: m/z 634.2 (M+H)⁺.

### 4-(5-((3,4-difluorobenzyl)carbamoyl)thiophen-2-yl)-2-isobutyl-5-(5-methyl-1,3,4-oxadiazol-2-yl)-6-(4-methylpentyl)nicotinamide (Compound 108)

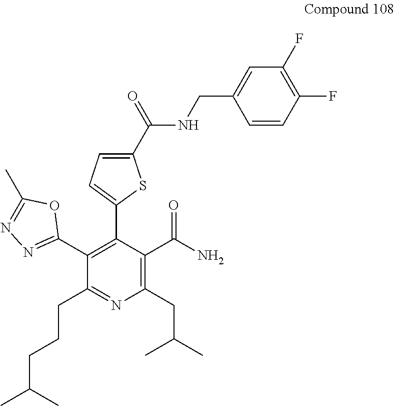 [SMILES: Cc1nnc(-c2c(CCCC(C)C)nc(CC(C)C)c(C(N)=O)c2-c2ccc(C(=O)NCc3ccc(F)c(F)c3)s2)o1]

Compound 108 was synthesized using a similar procedure described in the Example 4 above by using the appropriate materials.

¹H NMR (400 MHz, DMSO-d₆) δ 9.13 (t, J=5.99 Hz, 1H), 7.91 (s, 1H), 7.64 (d, J=3.91 Hz, 1H), 7.60 (s, 1H), 7.30-7.44 (m, 2H), 7.15 (s, 1H), 6.99 (d, J=3.79 Hz, 1H), 4.40 (d, J=5.87 Hz, 2H), 2.72 (d, J=7.09 Hz, 2H), 2.56-2.63 (m, 2H), 2.43 (s, 3H), 2.29-2.34 (m, 1H), 1.62 (q, J=7.67 Hz, 2H), 1.43-1.52 (m, 1H), 1.09-1.16 (m, 2H), 0.94 (d, J=6.60 Hz, 6H), 0.81 (d, J=6.60 Hz, 6H). LC-MS: m/z 596.2 (M+H)⁺.

### 6-(2-cyclohexylethyl)-4-(5-((3,4-difluorobenzyl)carbamoyl)thiophen-2-yl)-2-isobutyl-5-(5-methyl-1,3,4-oxadiazol-2-yl)nicotinamide (Compound 109)

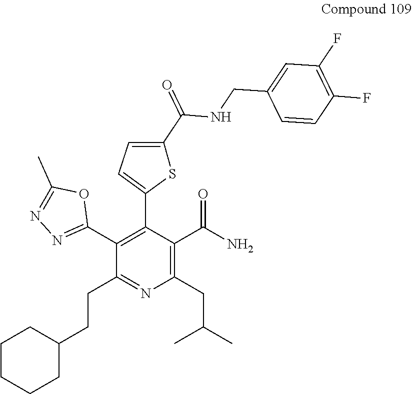 [SMILES: Cc1nnc(-c2c(CCC3CCCCC3)nc(CC(C)C)c(C(N)=O)c2-c2ccc(C(=O)NCc3ccc(F)c(F)c3)s2)o1]

Compound 109 was synthesized using a similar procedure described in the Example 4 above by using the appropriate materials.

¹H NMR (400 MHz, CDCl₃) δ 7.33 (d, J=3.76 Hz, 1H), 7.07-7.17 (m, 2H), 6.97-7.05 (m, 2H), 6.79 (t, J=6.02 Hz, 1H), 5.78 (d, J=17.57 Hz, 2H), 4.48 (d, J=6.02 Hz, 2H), 2.82 (d, J=7.28 Hz, 2H), 2.66-2.74 (m, 2H), 2.47 (s, 3H), 2.36 (d, J=6.78 Hz, 1H), 1.64 (s, 7H), 1.52 (d, J=9.03 Hz, 2H), 1.08-1.21 (m, 4H), 0.98 (d, J=6.53 Hz, 6H), 0.81-0.84 (m, 2H). LC-MS: m/z 622.3 (M+H)⁺.

### 4-(5-((3,4-difluorobenzyl)carbamoyl)thiophen-2-yl)-6-(4-fluorobenzyl)-2-isobutyl-5-(5-methyl-1,3,4-oxadiazol-2-yl)nicotinamide (Compound 110)

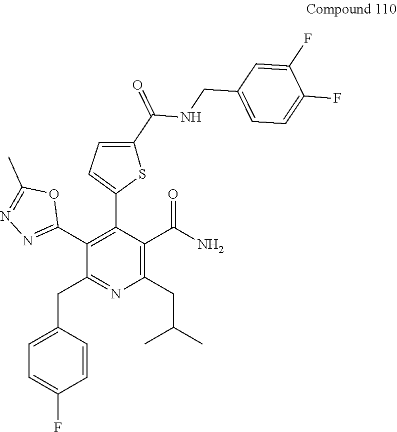 [SMILES: Cc1nnc(-c2c(Cc3ccc(F)cc3)nc(CC(C)C)c(C(N)=O)c2-c2ccc(C(=O)NCc3ccc(F)c(F)c3)s2)o1]

Compound 110 was synthesized using a similar procedure described in the Example 4 above by using the appropriate materials.

¹H NMR (400 MHz, CDCl₃) δ 7.34 (d, J=3.91 Hz, 1H), 7.08-7.19 (m, 2H), 6.97-7.07 (m, 4H), 6.85-6.93 (m, 2H), 6.37 (t, J=5.87 Hz, 1H), 5.51-5.70 (m, 2H), 4.51 (d, J=5.87 Hz, 2H), 4.20 (s, 2H), 2.86 (d, J=7.21 Hz, 2H), 2.35-2.44 (m, 1H), 2.32 (s, 3H), 0.99 (d, J=6.60 Hz, 6H). LC-MS: m/z 620.2 (M+H)⁺.

### 4-(5-((3,4-difluorobenzyl)carbamoyl)thiophen-2-yl)-2,6-diisobutyl-5-(5-methyl-1,3,4-oxadiazol-2-yl)nicotinamide (Compound 111)

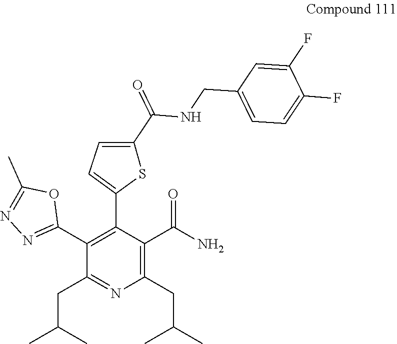 [SMILES: Cc1nnc(-c2c(CC(C)C)nc(CC(C)C)c(C(N)=O)c2-c2ccc(C(=O)NCc3ccc(F)c(F)c3)s2)o1]

Compound 111 was synthesized using a similar procedure described in the Example 4 above by using the appropriate materials.

¹H NMR (400 MHz, CD₃OD) (57.58 (d, J=4.52 Hz, 1H) 7.19-7.28 (m, 2H) 7.12-7.18 (m, 1H) 7.05-7.10 (m, 1H) 4.49 (s, 2H) 2.83-2.89 (m, 2H) 2.62-2.68 (m, 2H) 2.49 (s, 3H) 2.37-2.45 (m, 1H) 2.05-2.18 (m, 1H) 1.02 (d, J=6.60 Hz, 6H) 0.87-0.93 (m, 6H). LC-MS: m/z 568.2 (M+H)⁺.

### 4-[5-[(3,4-difluorophenyl)methylcarbamoyl]-2-thienyl]-6-ethyl-2-isobutyl-5-(5-methyl-1,3,4-oxadiazol-2-yl)pyridine-3-carboxamide (Compound 112)

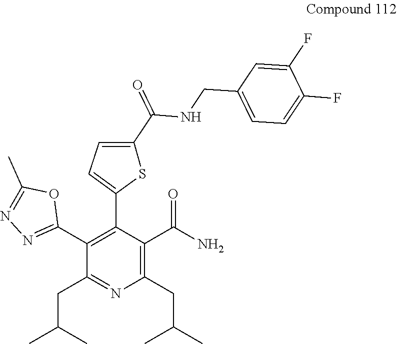 [SMILES: Cc1nnc(-c2c(CC(C)C)nc(CC(C)C)c(C(N)=O)c2-c2ccc(C(=O)NCc3ccc(F)c(F)c3)s2)o1]

Compound 112 was synthesized using a similar procedure described in the Example 4 above by using the appropriate materials.

¹H NMR (400 MHz, DMSO-d₆) (59.13-9.19 (m, 1H), 7.88-7.95 (m, 1H), 7.64 (d, J=3.79 Hz, 1H), 7.62 (s, 1H), 7.39 (d, J=10.76 Hz, 2H), 7.11-7.17 (m, 1H), 6.96-7.00 (m, 1H), 4.36-4.43 (m, 2H), 2.71 (s, 2H), 2.60-2.67 (m, 2H), 2.43 (s, 3H), 2.23-2.34 (m, 1H), 1.12-1.20 (m, 3H), 0.94 (d, J=6.60 Hz, 6H). LC-MS: m/z 540.2 (M+H)⁺.

### Example 5

### N-(m-methoxyphenyl)methyl-5-{5-carbamoyl-2-[2-(p-fluorophenyl)ethyl]-6-isobutyl-3-(5-methyl-1,3,4-oxadiazol-2-yl)-4-pyridyl}-2-thenamide (Compound 113)

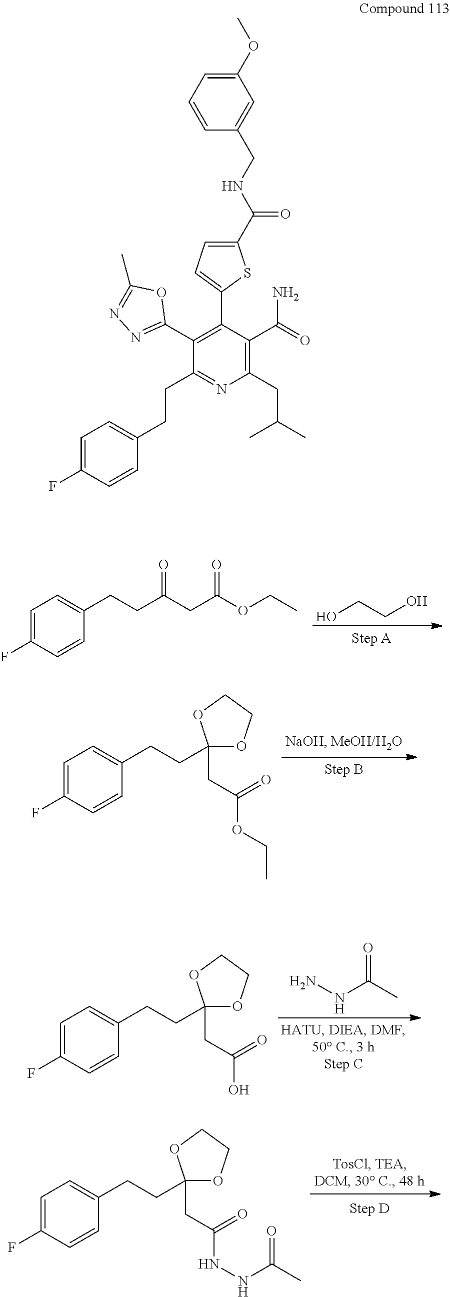

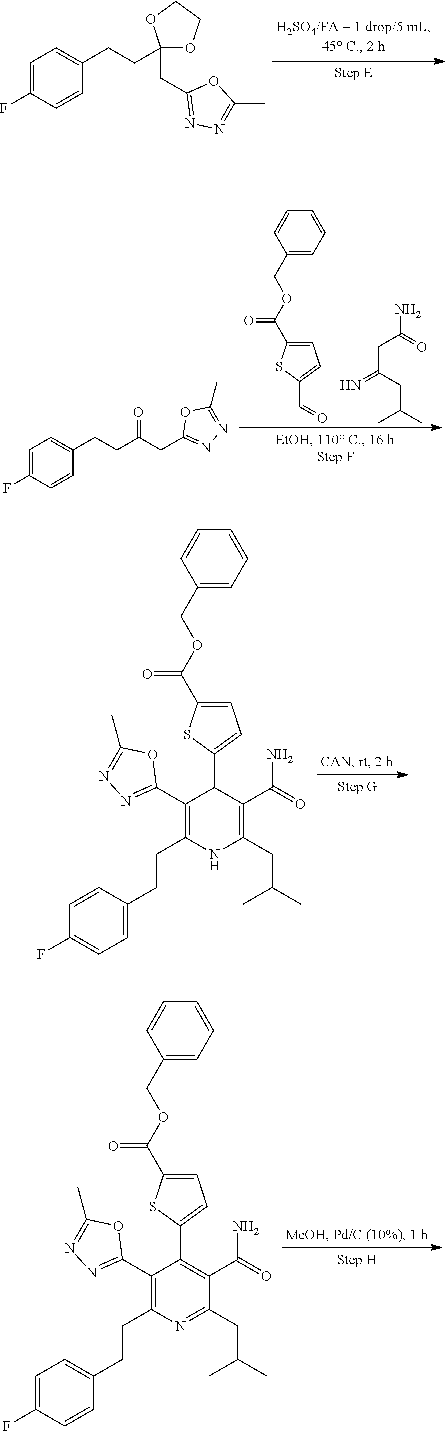

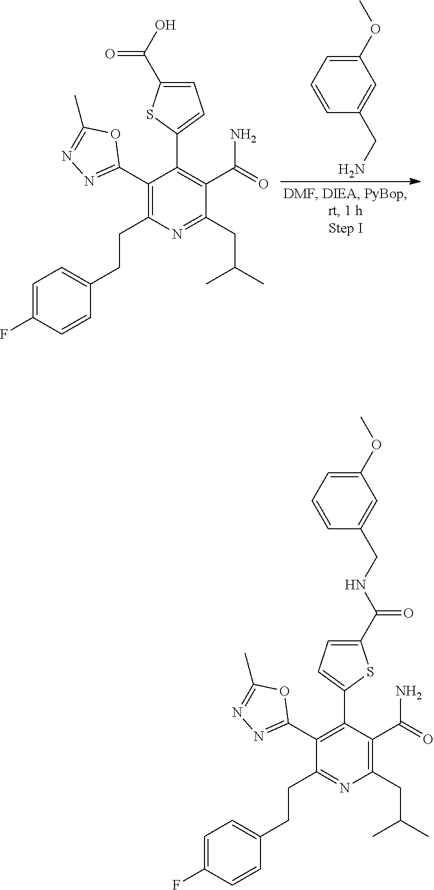

### Step A ethyl {2-[2-(4-fluorophenyl)ethyl]-1,3-dioxolan-2-yl}acetate

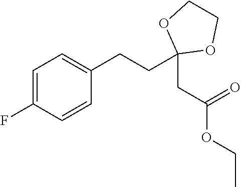 [SMILES: CCOC(=O)CC1(CCc2ccc(F)cc2)OCCO1]

To a solution of ethyl 5-(4-fluorophenyl)-3-oxopentanoate (14 g, 58.759 mmol) in toluene (2 mL) were added ethylene glycol (32.769 mL, 587.593 mmol) and TsOH·H₂O (0.08 g, 0.420 mmol), and the reaction mixture was stirred at 140° C. for 24 hrs. The reaction was diluted with EtOAc (300 ml) and water (300 ml). The organic layer was separated, washed with brine (300 ml) and then concentrated in vacuo. The residue was purified by silica gel column chromatography eluting with ethyl acetate in petroleum ether (PE:EtOAc=10:1). The organic layer was concentrated in vacuo to give ethyl {2-[2-(4-fluorophenyl)ethyl]-1,3-dioxolan-2-yl}acetate (3.7 g, 22.30%). LC-MS: m/z 305.1 (M+Na)⁺.

### Step B {2-[2-(4-fluorophenyl)ethyl]-1,3-dioxolan-2-yl}acetic acid

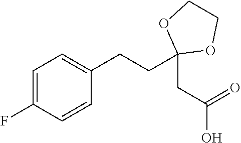 [SMILES: O=C(O)CC1(CCc2ccc(F)cc2)OCCO1]

To a solution of ethyl {2-[2-(4-fluorophenyl)ethyl]-1,3-dioxolan-2-yl}acetate (3.7 g, 13.106 mmol) in MeOH (40 mL) and H₂O (15 mL) was added NaOH (2.10 g, 52.424 mmol). The reaction mixture was stirred at room temperature for 3 hrs. 1 N HCl aq. (60 mL) was added into the reaction mixture and the mixture was diluted with EtOAc (100 mL) and water (50 mL). The organic layer was separated, washed with water (50 mL×2) and brine (50 mL), dried over Na₂SO₄ and then filtered. The organic layer was collected, concentrated in vacuo to give {2-[2-(4-fluorophenyl)ethyl]-1,3-dioxolan-2-yl}acetic acid (3 g, 90.03%). LC-MS: m/z 277.1 (M+H)⁺.

### Step C N′-acetyl-2-(2-(4-fluorophenethyl)-1,3-dioxolan-2-yl)acetohydrazide

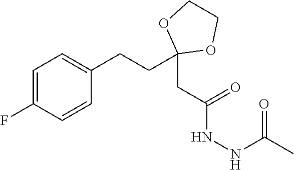 [SMILES: CC(=O)NNC(=O)CC1(CCc2ccc(F)cc2)OCCO1]

To a solution of {2-[2-(4-fluorophenyl)ethyl]-1,3-dioxolan-2-yl}acetic acid (3 g, 11.799 mmol) in DMF (30 mL) were added acetohydrazide (1.75 g, 23.598 mmol), DIEtOAc (4.57 g, 35.397 mmol) and HATU (5.38 g, 14.159 mmol), the reaction mixture was stirred at 50° C. for 3 hrs. The reaction mixture was cooled down to room temperature, diluted with EtOAc (50 mL) and water (20 mL). The organic layer was separated, washed with water (20 mL×2) and brine (20 mL). The organic layer was separated, dried over Na₂SO₄ and then filtered. The organic layer was collected, concentrated in vacuo. The residue was purified by silica gel column chromatography eluting with PE:EtOAc=1:1. The organic layer was collected, concentrated in vacuo, and dried to give N′-acetyl-2-(2-(4-fluorophenethyl)-1,3-dioxolan-2-yl)acetohydrazide (2.8 g, 76.47%). LC-MS: m/z 310.9 (M+H)⁺.

### Step D 2-({2-[2-(4-fluorophenyl)ethyl]-1,3-dioxolan-2-yl}methyl)-5-methyl-1,3,4-oxadiazole

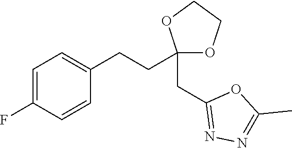 [SMILES: Cc1nnc(CC2(CCc3ccc(F)cc3)OCCO2)o1]

To a solution of N′-acetyl-2-(2-(4-fluorophenethyl)-1,3-dioxolan-2-yl)acetohydrazide (2.5 g, 8.056 mmol) in DCM (30 mL) were added TEA (1.456 mL, 10.473 mmol) and 4-toluene sulfonyl chloride (1.84 g, 9.667 mmol) and the reaction mixture was stirred at 30° C. for 48 hrs. The mixture was diluted with DCM (100 mL) and water (50 mL). The organic layer was separated, washed with further water (50 mL) and concentrated in vacuo. The residue was purified by silica gel column chromatography eluting with DCM:MeOH=20:1. The organic layer was collected, concentrated in vacuo to give 2-({2-[2-(4-fluorophenyl)ethyl]-1,3-dioxolan-2-yl}methyl)-5-methyl-1,3,4-oxadiazole (1.8 g, 76.44%). LC-MS: m/z 293.1 (M+H)⁺.

### Step E 4-(4-fluorophenyl)-1-(5-methyl-1,3,4-oxadiazol-2-yl)butan-2-one

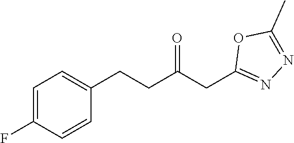 [SMILES: Cc1nnc(CC(=O)CCc2ccc(F)cc2)o1]

To a solution of 2-({2-[2-(4-fluorophenyl)ethyl]-1,3-dioxolan-2-yl}methyl)-5-methyl-1,3,4-oxadiazole (1.8 g, 6.158 mmol) in HCOOH (5 mL) were added H₂SO₄ (0.02 mL), and the reaction mixture was stirred at 45° C. for 2 hrs. The reaction mixture was cooled down to room temperature, diluted with EtOAc (100 mL) and water (50 mL). The organic layer was separated, washed with further water (50 mL×2). Then the organic layer was dried over Na₂SO₄, filtered and concentrated in vacuo. The residue was purified by silica gel column chromatography eluting with ethyl acetate in petroleum ether (PE:EtOAc=3:1). The organic layer was collected, concentrated in vacuo to give 4-(4-fluorophenyl)-1-(5-methyl-1,3,4-oxadiazol-2-yl)butan-2-one (1.2 g, 78.50%). LC-MS: m/z 249.1 (M+H)⁺.

### Step F benzyl 5-{3-carbamoyl-6-[2-(4-fluorophenyl)ethyl]-5-(5-methyl-1,3,4-oxadiazol-2-yl)-2-(2-methylpropyl)-1,4-dihydropyridin-4-yl}thiophene-2-carboxylate

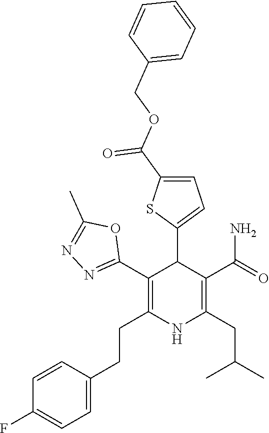 [SMILES: Cc1nnc(C2=C(CCc3ccc(F)cc3)NC(CC(C)C)=C(C(N)=O)C2c2ccc(C(=O)OCc3ccccc3)s2)o1]

A solution of benzyl 5-formylthiophene-2-carboxylate (238.09 mg, 0.967 mmol) in EtOH (3 mL) were added 4-(4-fluorophenyl)-1-(5-methyl-1,3,4-oxadiazol-2-yl)butan-2-one (240 mg, 0.967 mmol) and 3-azanylidene-5-methylhexanamide (151.22 mg, 1.063 mmol), and the reaction mixture was stirred at 110° C. for 18 hrs. The reaction mixture was cooled down to room temperature, concentrated and used directly without further work up (600 mg, crude). LC-MS: m/z 601.2 (M+H)⁺.

### Step G benzyl 5-{3-carbamoyl-6-[2-(4-fluorophenyl)ethyl]-5-(5-methyl-1,3,4-oxadiazol-2-yl)-2-(2-methylpropyl)pyridin-4-yl}thiophene-2-carboxylate

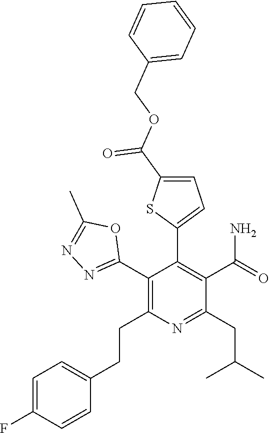 [SMILES: Cc1nnc(-c2c(CCc3ccc(F)cc3)nc(CC(C)C)c(C(N)=O)c2-c2ccc(C(=O)OCc3ccccc3)s2)o1]

A solution of benzyl 5-{3-carbamoyl-6-[2-(4-fluorophenyl)ethyl]-5-(5-methyl-1,3,4-oxadiazol-2-yl)-2-(2-methylpropyl)-1,4-dihydropyridin-4-yl}thiophene-2-carboxylate (600 mg, 0.999 mmol) in EtOH (3 mL) was added CAN (821.37 mg, 1.498 mmol), and the reaction mixture was stirred at room temperature for 2 hrs. The mixture was diluted with EtOAc (50 mL) and water (20 mL). The organic layer was separated, washed with water (20 mL×2) and brine (20 mL). The organic layer was separated, dried over Na₂SO₄ and then filtered. The organic layer was collected, concentrated in vacuo. The residue was purified by using silica gel column chromatography eluting with PE:EtOAc=1:1 to give benzyl 5-{3-carbamoyl-6-[2-(4-fluorophenyl)ethyl]-5-(5-methyl-1,3,4-oxadiazol-2-yl)-2-(2-methylpropyl)pyridin-4-yl}thiophene-2-carboxylate (500 mg, 83.52%). LC-MS: m/z 599.2 (M+H)⁺.

### Step H 5-{5-carbamoyl-2-[2-(4-fluorophenyl)ethyl]-3-(5-methyl-1,3,4-oxadiazol-2-yl)-6-(2-methylpropyl)pyridin-4-yl}thiophene-2-carboxylic acid

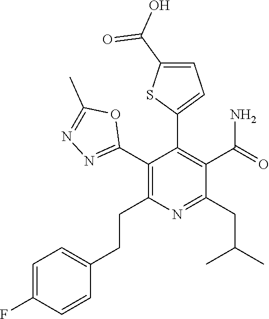 [SMILES: Cc1nnc(-c2c(CCc3ccc(F)cc3)nc(CC(C)C)c(C(N)=O)c2-c2ccc(C(=O)O)s2)o1]

To a solution of benzyl 5-{5-carbamoyl-2-[2-(4-fluorophenyl)ethyl]-3-(5-methyl-1,3,4-oxadiazol-2-yl)-6-(2-methylpropyl)pyridin-4-yl}thiophene-2-carboxylate (500 mg, 0.835 mmol) in MeOH (5 mL) was added Pd/C (200 mg, 1.879 mmol, 10% Pd, wetted with ca. 55% Water), and the reaction mixture was stirred at 25° C. for 1 hr under H₂ (15 Psi). The reaction mixture was filtered and concentrated in vacuo to give 5-{5-carbamoyl-2-[2-(4-fluorophenyl)ethyl]-3-(5-methyl-1,3,4-oxadiazol-2-yl)-6-(2-methylpropyl)pyridin-4-yl}thiophene-2-carboxylic acid (390 mg, 91.82%). LC-MS: m/z 509.1 (M+H)⁺.

### Step I N-(m-methoxyphenyl)methyl-5-{5-carbamoyl-2-[2-(p-fluorophenyl)ethyl]-6-isobutyl-3-(5-methyl-1,3,4-oxadiazol-2-yl)-4-pyridyl}-2-thenamide (Compound 113)

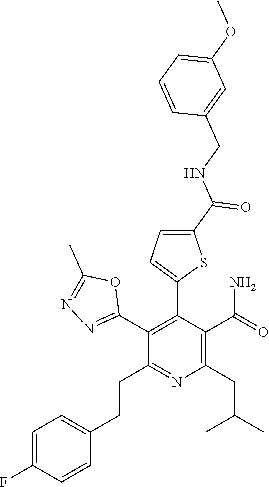 [SMILES: COc1cccc(CNC(=O)c2ccc(-c3c(C(N)=O)c(CC(C)C)nc(CCc4ccc(F)cc4)c3-c3nnc(C)o3)s2)c1]

To a solution of 5-{5-carbamoyl-2-[2-(4-fluorophenyl)ethyl]-3-(5-methyl-1,3,4-oxadiazol-2-yl)-6-(2-methylpropyl)pyridin-4-yl}thiophene-2-carboxylic acid (15 mg, 0.029 mmol) in DMF (1 mL) were added (3-methoxyphenyl)methanamine (6.07 mg, 0.044 mmol), DIEA (11.44 mg, 0.088 mmol) and PyBOP (23.02 mg, 0.044 mmol), the reaction mixture was stirred at room temperature for 1 hr. The mixture was diluted with EtOAc (10 mL), washed with water (30 mL*2). The organic layers were dried over Na₂SO₄, filtered and concentrated under vacuum to dryness. The residue was purified by Prep-HPLC to give N-(m-methoxyphenyl)methyl-5-{5-carbamoyl-2-[2-(p-fluorophenyl)ethyl]-6-isobutyl-3-(5-methyl-1,3,4-oxadiazol-2-yl)-4-pyridyl}-2-thenamide (2.03 mg, 10.96%).

¹H NMR (400 MHz, DMSO-d₆) δ 9.07 (t, J=6.0 Hz, 1H), 7.88 (s, 1H), 7.65 (d, J=3.6 Hz, 1H), 7.58 (s, 1H), 7.25 (t, J=8.0 Hz, 1H), 7.02-7.14 (m, 4H), 6.97 (d, J=4.0 Hz, 1H), 6.78-6.90 (m, 3H), 4.40 (d, J=5.6 Hz, 2H), 3.74 (s, 3H), 2.93-3.04 (m, 4H), 2.73 (d, J=6.8 Hz, 2H), 2.38 (s, 3H), 2.26-2.35 (m, 1H), 0.94 (d, J=6.8 Hz, 6H). ¹⁹F NMR (377 MHz, DMSO-d₆) δ −117.27. LC-MS: m/z 628.1 (M+H)⁺.

### N-(p-methoxyphenyl)methyl-5-{5-carbamoyl-2-[2-(p-fluorophenyl)ethyl]-6-isobutyl-3-(5-methyl-1,3,4-oxadiazol-2-yl)-4-pyridyl}-2-thenamide (Compound 114)

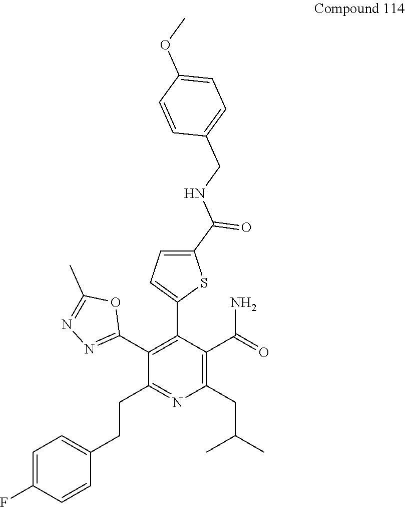 [SMILES: COc1ccc(CNC(=O)c2ccc(-c3c(C(N)=O)c(CC(C)C)nc(CCc4ccc(F)cc4)c3-c3nnc(C)o3)s2)cc1]

Compound 114 was synthesized using a similar procedure described in the Example 5 above by using the appropriate materials.

¹H NMR (400 MHz, DMSO-d₆) δ 9.03 (t, J=5.6 Hz, 1H), 7.89 (s, 1H), 7.63 (d, J=3.6 Hz, 1H), 7.59 (s, 1H), 7.23 (d, J=8.0 Hz, 2H), 7.01-7.15 (m, 4H), 6.96 (d, J=3.6 Hz, 1H), 6.90 (d, J=8.4 Hz, 2H), 4.35 (d, J=6.0 Hz, 2H), 3.73 (s, 3H), 2.91-3.03 (m, 4H), 2.73 (d, J=6.8 Hz, 2H), 2.38 (s, 3H), 2.23-2.35 (m, 1H), 0.94 (d, J=6.4 Hz, 6H). ¹⁹F NMR (377 MHz, DMSO-d₆) δ −117.26. LC-MS: m/z 628.2 (M+H)⁺.

### N-benzyl-5-{5-carbamoyl-2-[2-(p-fluorophenyl)ethyl]-6-isobutyl-3-(5-methyl-1,3,4-oxadiazol-2-yl)-4-pyridyl}-2-thenamide (Compound 115)

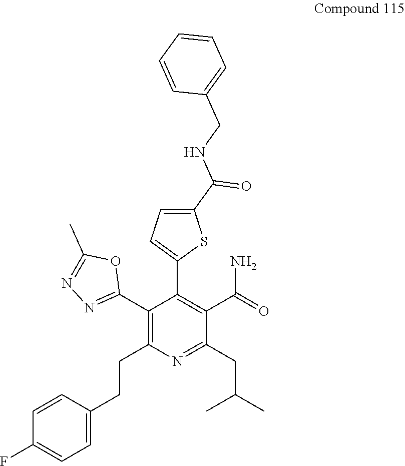 [SMILES: Cc1nnc(-c2c(CCc3ccc(F)cc3)nc(CC(C)C)c(C(N)=O)c2-c2ccc(C(=O)NCc3ccccc3)s2)o1]

Compound 115 was synthesized using a similar procedure described in the Example 5 above by using the appropriate materials.

¹H NMR (400 MHz, DMSO-d₆) δ 9.09 (t, J=5.6 Hz, 1H), 7.88 (s, 1H), 7.65 (d, J=4.0 Hz, 1H), 7.59 (br s, 1H), 7.22-7.37 (m, 5H), 7.01-7.14 (m, 4H), 6.96 (d, J=4.0 Hz, 1H), 4.43 (d, J=6.0 Hz, 2H), 2.92-3.01 (m, 4H), 2.73 (d, J=7.2 Hz, 2H), 2.38 (s, 3H), 2.23-2.35 (m, 1H), 0.94 (d, J=6.4 Hz, 6H). ¹⁹F NMR (377 MHz, DMSO-d₆) δ −117.27. LC-MS: m/z 598.2 (M+H)⁺.

### N-[(3-fluoro-5-methoxyphenyl)methyl]-5-{5-carbamoyl-2-[2-(p-fluorophenyl)ethyl]-6-isobutyl-3-(5-methyl-1,3,4-oxadiazol-2-yl)-4-pyridyl}-2-thenamide (Compound 116)

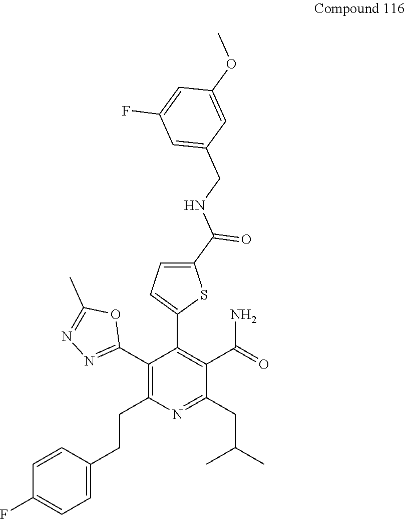 [SMILES: COc1cc(F)cc(CNC(=O)c2ccc(-c3c(C(N)=O)c(CC(C)C)nc(CCc4ccc(F)cc4)c3-c3nnc(C)o3)s2)c1]

Compound 116 was synthesized using a similar procedure described in the Example 5 above by using the appropriate materials.

¹H NMR (400 MHz, DMSO-d₆) δ 9.10 (t, J=6.0 Hz, 1H), 7.89 (br s, 1H), 7.66 (d, J=3.6 Hz, 1H), 7.59 (br s, 1H), 7.01-7.16 (m, 4H), 6.97 (d, J=4.0 Hz, 1H), 6.63-6.76 (m, 3H), 4.39 (d, J=6.0 Hz, 2H), 3.75 (s, 3H), 2.90-3.05 (m, 4H), 2.73 (d, J=7.2 Hz, 2H), 2.37 (s, 3H), 2.25-2.35 (m, 1H), 0.94 (d, J=6.4 Hz, 6H). ¹⁹F NMR (377 MHz, DMSO-d₆) δ −111.85, −117.26. LC-MS: m/z 646.1 (M+H)⁺.

### N-[(4-fluoro-3-methoxyphenyl)methyl]-5-{5-carbamoyl-2-[2-(p-fluorophenyl)ethyl]-6-isobutyl-3-(5-methyl-1,3,4-oxadiazol-2-yl)-4-pyridyl}-2-thenamide (Compound 117)

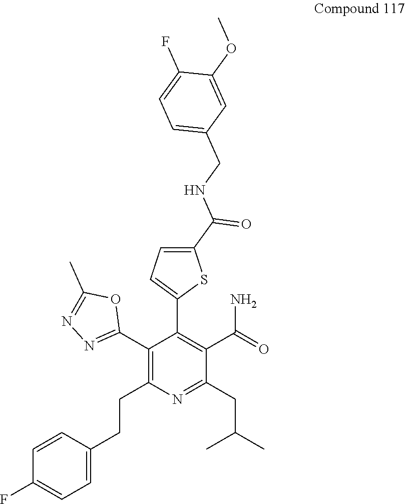 [SMILES: COc1cc(CNC(=O)c2ccc(-c3c(C(N)=O)c(CC(C)C)nc(CCc4ccc(F)cc4)c3-c3nnc(C)o3)s2)ccc1F]

Compound 117 was synthesized using a similar procedure described in the Example 5 above by using the appropriate materials.

¹H NMR (400 MHz, DMSO-d₆) δ 9.07 (t, J=6.0 Hz, 1H), 7.89 (br s, 1H), 7.65 (d, J=4.0 Hz, 1H), 7.59 (br s, 1H), 7.00-7.20 (m, 6H), 6.96 (d, J=3.6 Hz, 1H), 6.81-6.87 (m, 1H), 4.39 (d, J=6.0 Hz, 2H), 3.82 (s, 3H), 2.90-3.02 (m, 4H), 2.73 (d, J=7.2 Hz, 2H), 2.38 (s, 3H), 2.25-2.34 (m, 1H), 0.91 (d, J=6.8 Hz, 6H). ¹⁹F NMR (377 MHz, DMSO-d₆) δ −117.27, −137.96. LC-MS: m/z 646.2 (M+H)⁺.

### (S)-4-(5-((2,3-dihydro-1H-inden-1-yl)carbamoyl)thiophen-2-yl)-6-(4-fluorophenethyl)-2-isobutyl-5-(5-methyl-1,3,4-oxadiazol-2-yl)nicotinamide (Compound 118)

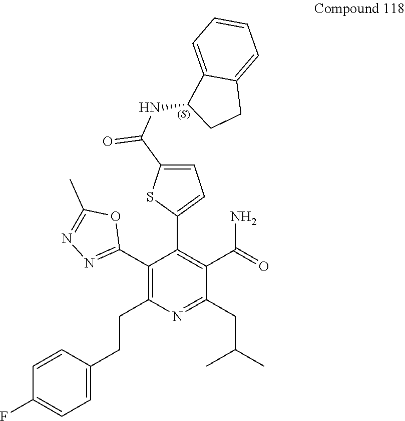

Compound 118 was synthesized using a similar procedure described in the Example 5 above by using the appropriate materials.

¹H NMR (400 MHz, DMSO-d₆) δ 8.86 (d, J=8.4 Hz, 1H), 7.89 (s, 1H), 7.67 (d, J=3.6 Hz, 1H), 7.60 (s, 1H), 7.15-7.33 (m, 4H), 7.00-7.15 (m, 4H), 6.94 (d, J=4.0 Hz, 1H), 5.42-5.52 (m, 1H), 2.93-3.06 (m, 5H), 2.78-2.90 (m, 1H), 2.73 (d, J=7.2 Hz, 2H), 2.37-2.46 (m, 4H), 2.22-2.35 (m, 1H), 1.85-2.01 (m, 1H), 0.94 (d, J=6.8 Hz, 6H). ¹⁹F NMR (377 MHz, DMSO-d₆) δ −117.26. LC-MS: m/z 624.2 (M+H)⁺.

### (R)-4-(5-((4-fluoro-2,3-dihydro-1H-inden-1-yl)carbamoyl)thiophen-2-yl)-6-(4-fluorophenethyl)-2-isobutyl-5-(5-methyl-1,3,4-oxadiazol-2-yl)nicotinamide (Compound 119)

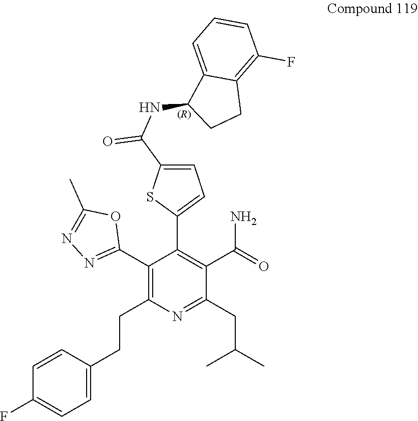

Compound 119 was synthesized using a similar procedure described in the Example 5 above by using the appropriate materials.

¹H NMR (400 MHz, DMSO-d₆) δ 8.90 (d, J=8.4 Hz, 1H), 7.89 (s, 1H), 7.66 (d, J=4.0 Hz, 1H), 7.60 (s, 1H), 7.20-7.32 (m, 1H), 7.01-7.15 (m, 6H), 6.95 (d, J=4.0 Hz, 1H), 5.45-5.55 (m, 1H), 2.92-3.11 (m, 5H), 2.79-2.90 (m, 1H), 2.73 (d, J=6.8 Hz, 2H), 2.45-2.48 (m, 1H), 2.40 (s, 3H), 2.23-2.35 (m, 1H), 1.93-2.07 (m, 1H), 0.94 (d, J=6.4 Hz, 6H). ¹⁹F NMR (377 MHz, DMSO-d₆) δ −117.26, −118.80. LC-MS: m/z 642.3 (M+H)⁺.

### (R)-4-(5-((5-fluoro-2,3-dihydro-1H-inden-1-yl)carbamoyl)thiophen-2-yl)-6-(4-fluorophenethyl)-2-isobutyl-5-(5-methyl-1,3,4-oxadiazol-2-yl)nicotinamide (Compound 120)

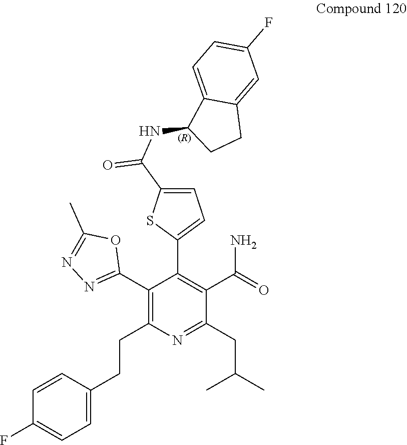

Compound 120 was synthesized using a similar procedure described in the Example 5 above by using the appropriate materials.

¹H NMR (400 MHz, DMSO-d₆) δ 8.84 (d, J=8.0 Hz, 1H), 7.89 (br s, 1H), 7.66 (d, J=4.0 Hz, 1H), 7.59 (br s, 1H), 7.19-7.27 (m, 1H), 6.97-7.15 (m, 6H), 6.94 (d, J=3.6 Hz, 1H), 5.39-5.45 (m, 1H), 2.93-3.05 (m, 5H), 2.80-2.90 (m, 1H), 2.73 (d, J=7.2 Hz, 2H), 2.43-2.48 (m, 1H), 2.40 (s, 3H), 2.22-2.36 (m, 1H), 1.91-2.04 (m, 1H), 0.94 (d, J=6.8 Hz, 6H). ¹⁹F NMR (377 MHz, DMSO-d₆) δ −115.77, −117.27. LC-MS: m/z 642.2 (M+H)⁺.

### (R)-4-(5-((6-fluoro-2,3-dihydro-1H-inden-1-yl)carbamoyl)thiophen-2-yl)-6-(4-fluorophenethyl)-2-isobutyl-5-(5-methyl-1,3,4-oxadiazol-2-yl)nicotinamide (Compound 121)

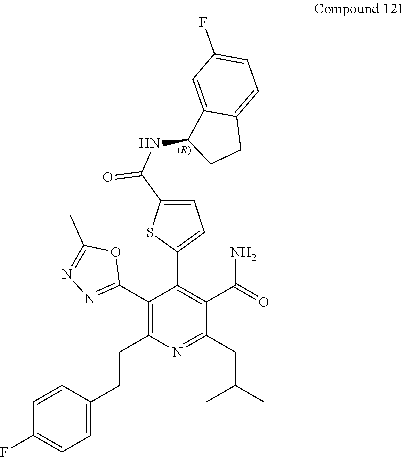

Compound 121 was synthesized using a similar procedure described in the Example 5 above by using the appropriate materials.

¹H NMR (400 MHz, DMSO-d₆) δ 8.88 (d, J=8.4 Hz, 1H), 7.89 (br s, 1H), 7.67 (d, J=4.0 Hz, 1H), 7.59 (br s, 1H), 7.25-7.32 (m, 1H), 6.98-7.15 (m, 6H), 6.95 (d, J=4.0 Hz, 1H), 5.41-5.45 (m, 1H), 2.90-3.03 (m, 5H), 2.77-2.86 (m, 1H), 2.73 (d, J=7.2 Hz, 2H), 2.43-2.48 (m, 1H), 2.40 (s, 3H), 2.23-2.35 (m, 1H), 1.91-2.02 (m, 1H), 0.94 (d, J=6.4 Hz, 6H). ¹⁹F NMR (377 MHz, DMSO-d₆) δ −116.96,−117.27. ¹⁹F NMR (377 MHz, DMSO-d₆) δ −116.96, −117.27. LC-MS: m/z 642.4 (M+H)⁺.

### (R)-6-(4-fluorophenethyl)-2-isobutyl-4-(5-((4-methoxy-2,3-dihydro-1H-inden-1-yl)carbamoyl)thiophen-2-yl)-5-(5-methyl-1,3,4-oxadiazol-2-yl)nicotinamide (Compound 122)

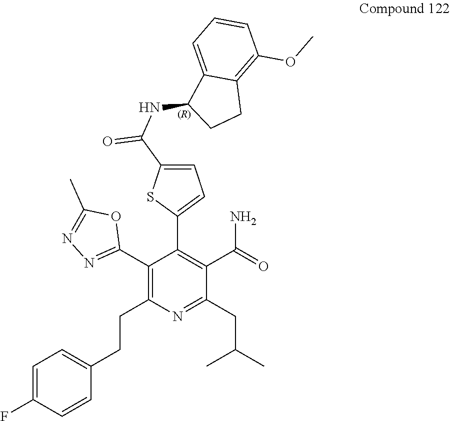

Compound 122 was synthesized using a similar procedure described in the Example 5 above by using the appropriate materials.

¹H NMR (400 MHz, DMSO-d₆) δ 8.85 (d, J=8.4 Hz, 1H), 7.90 (s, 1H), 7.67 (d, J=4.0 Hz, 1H), 7.60 (s, 1H), 7.19 (t, J=8.0 Hz, 1H), 7.01-7.15 (m, 4H), 6.95 (d, J=3.6 Hz, 1H), 6.79-6.90 (m, 2H), 5.40-5.50 (m, 1H), 3.80 (s, 3H), 2.88-3.01 (m, 5H), 2.64-2.78 (m, 3H), 2.37-2.48 (m, 4H), 2.24-2.36 (m, 1H), 1.86-1.99 (m, 1H), 0.94 (d, J=6.8 Hz, 6H). ¹⁹F NMR (377 MHz, DMSO-d₆) δ −117.26. LC-MS: m/z 654.2 (M+H)⁺.

### (R)-4-(5-((6,7-dihydro-5H-cyclopenta[b]pyridin-5-yl)carbamoyl)thiophen-2-yl)-6-(4-fluorophenethyl)-2-isobutyl-5-(5-methyl-1,3,4-oxadiazol-2-yl)nicotinamide (Compound 123)

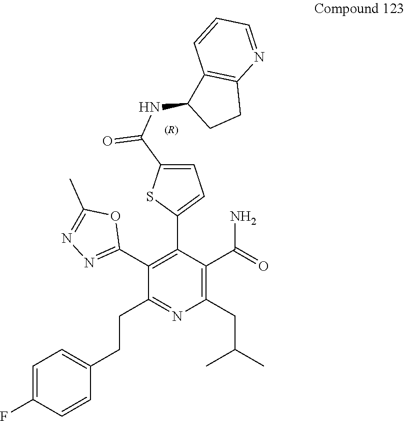

Compound 123 was synthesized using a similar procedure described in the Example 5 above by using the appropriate materials.

¹H NMR (400 MHz, DMSO-d₆) δ 8.92 (d, J=8.0 Hz, 1H), 8.40 (d, J=4.4 Hz, 1H), 7.90 (s, 1H), 7.56-7.69 (m, 3H), 7.00-7.23 (m, 5H), 6.95 (d, J=4.0 Hz, 1H), 5.43-5.57 (m, 1H), 2.86-3.09 (m, 7H), 2.73 (d, J=6.8 Hz, 2H), 2.40 (s, 3H), 2.24-2.35 (m, 1H), 1.91-2.05 (m, 1H), 0.94 (d, J=6.4 Hz, 6H). ¹⁹F NMR (377 MHz, DMSO-d₆) δ −117.26. LC-MS: m/z 625.4 (M+H)⁺.

### (S)-4-(5-((6,7-dihydro-5H-cyclopenta[b]pyridin-5-yl)carbamoyl)thiophen-2-yl)-6-(4-fluorophenethyl)-2-isobutyl-5-(5-methyl-1,3,4-oxadiazol-2-yl)nicotinamide (Compound 124)

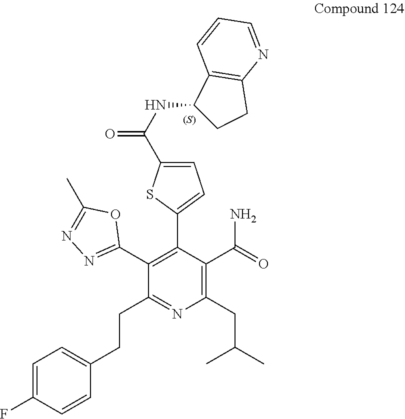

Compound 124 was synthesized using a similar procedure described in the Example 5 above by using the appropriate materials.

¹H NMR (400 MHz, DMSO-d₆) δ 8.91 (d, J=8.0 Hz, 1H), 8.40 (d, J=4.8 Hz, 1H), 7.89 (s, 1H), 7.56-7.67 (m, 3H), 7.19 (dd, J=5.2 Hz, J=7.6 Hz, 1H), 7.02-7.13 (m, 4H), 6.95 (d, J=3.6 Hz, 1H), 5.43-5.55 (m, 1H), 2.91-3.00 (m, 7H), 2.73 (d, J=7.2 Hz, 2H), 2.40 (s, 3H), 2.25-2.35 (m, 1H), 1.94-2.01 (m, 1H), 0.94 (d, J=6.8 Hz, 6H). ¹⁹F NMR (377 MHz, DMSO-d₆) δ −117.26. LC-MS: m/z 625.2 (M+H)⁺.

### Example 6

### N-(3,4-difluorophenyl)methyl-2-{5-carbamoyl-2-[2-(p-fluorophenyl)ethyl]-6-isobutyl-3-(5-methyl-1,3,4-oxadiazol-2-yl)-4-pyridyl}-1,3-thiazole-5-carboxamide (Compound 125)

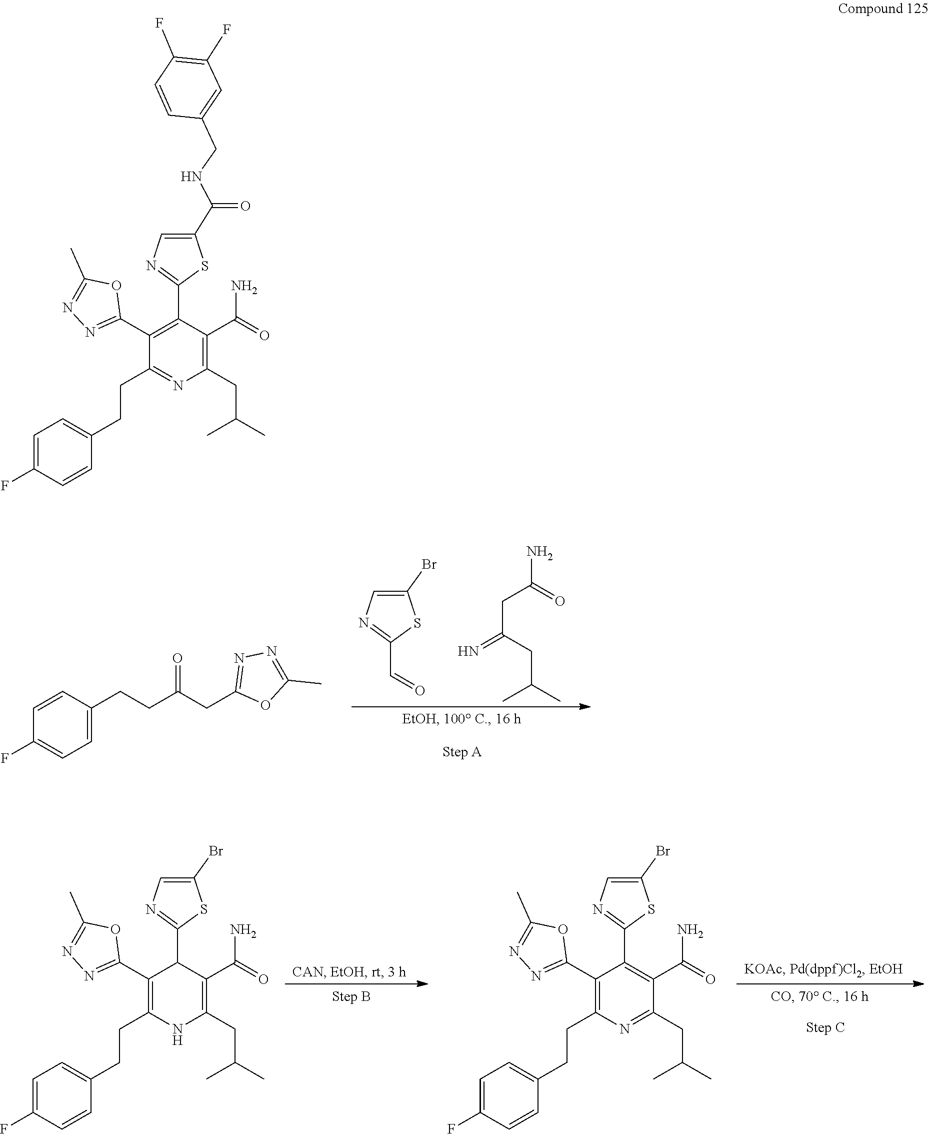

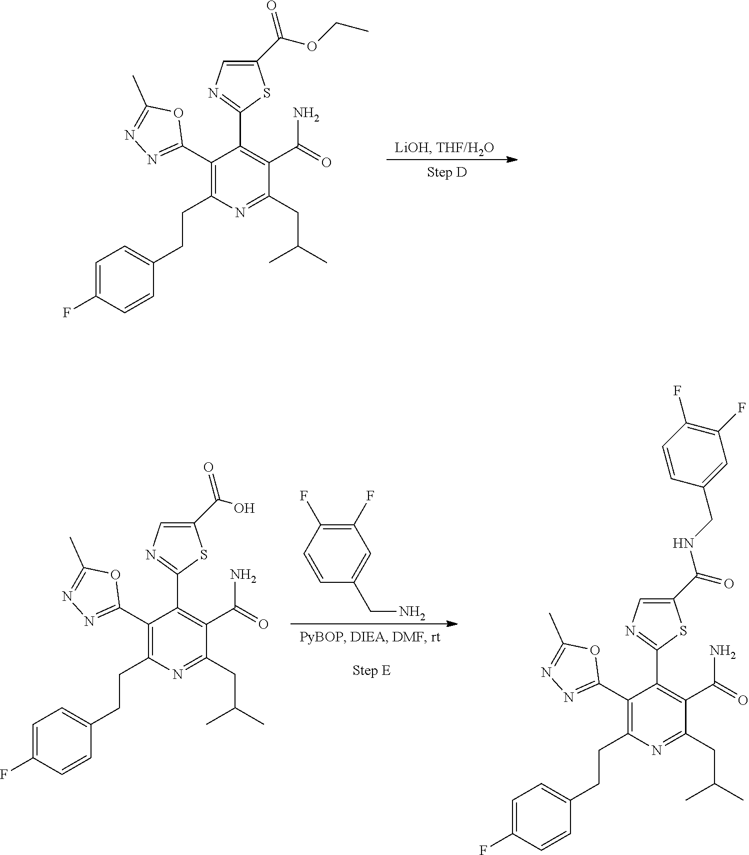 [SMILES: CCOC(=O)c1cnc(-c2c(C(N)=O)c(CC(C)C)nc(CCc3ccc(F)cc3)c2-c2nnc(C)o2)s1 ; Cc1nnc(-c2c(CCc3ccc(F)cc3)nc(CC(C)C)c(C(N)=O)c2-c2ncc(C(=O)O)s2)o1 ; NCc1ccc(F)c(F)c1 ; Cc1nnc(-c2c(CCc3ccc(F)cc3)nc(CC(C)C)c(C(N)=O)c2-c2ncc(C(=O)NCc3ccc(F)c(F)c3)s2)o1]

### Step A The synthesis of 4-(5-bromothiazol-2-yl)-6-(4-fluorophenethyl)-2-isobutyl-5-(5-methyl-1,3,4-oxadiazol-2-yl)-1,4-dihydropyridine-3-carboxamide

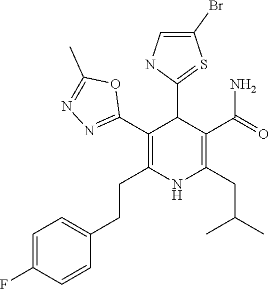

To a solution of 4-(4-fluorophenyl)-1-(5-methyl-1,3,4-oxadiazol-2-yl)butan-2-one (50 mg, 0.202 mmol) in EtOH (2 mL) was added 5-bromothiazole-2-carbaldehyde (39 mg, 0.202 mmol) and 3-imino-5-methylhexanamide (29 mg, 0.202 mmol) at room temperature, and the reaction mixture was stirred at 100° C. for 16 hours. The reaction mixture was concentrated in vacuo to afford the 4-(5-bromothiazol-2-yl)-6-(4-fluorophenethyl)-2-isobutyl-5-(5-methyl-1,3,4-oxadiazol-2-yl)-1,4-dihydropyridine-3-carboxamide (180 mg, crude). LC-MS: m/z 545.7 (M+H)⁺.

### Step B The synthesis of 4-(5-bromothiazol-2-yl)-6-(4-fluorophenethyl)-2-isobutyl-5-(5-methyl-1,3,4-oxadiazol-2-yl)nicotinamide

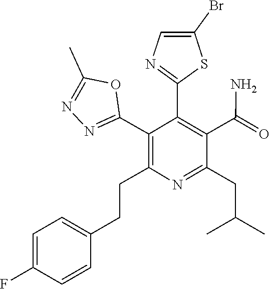 [SMILES: Cc1nnc(-c2c(CCc3ccc(F)cc3)nc(CC(C)C)c(C(N)=O)c2-c2ncc(Br)s2)o1]

To a solution of 4-(5-bromothiazol-2-yl)-6-(4-fluorophenethyl)-2-isobutyl-5-(5-methyl-1,3,4-oxadiazol-2-yl)-1,4-dihydropyridine-3-carboxamide (180 mg, crude) in EtOH (3 mL) were added diammonium cerium (IV) nitrate (221 mg, 0.404 mmol) and the reaction mixture was stirred at room temperature for 3 hours. The reaction mixture was concentrated in vacuo. The residue was purified using silica gel column chromatography eluting with 30% ethyl acetate in petroleum ether to afford the 4-(5-bromothiazol-2-yl)-6-(4-fluorophenethyl)-2-isobutyl-5-(5-methyl-1,3,4-oxadiazol-2-yl)nicotinamide (50 mg, 45.9% yield). LC-MS: m/z 544.0 (M+H)⁺.

### Step C ethyl 2-(3-carbamoyl-6-(4-fluorophenethyl)-2-isobutyl-5-(5-methyl-1,3,4-oxadiazol-2-yl)pyridin-4-yl)thiazole-5-carboxylate

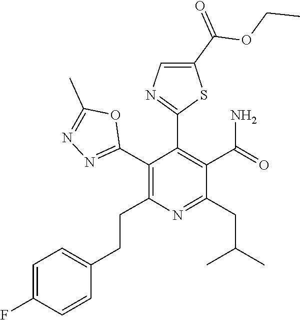 [SMILES: CCOC(=O)c1cnc(-c2c(C(N)=O)c(CC(C)C)nc(CCc3ccc(F)cc3)c2-c2nnc(C)o2)s1]

A mixture of 4-(5-bromothiazol-2-yl)-6-(4-fluorophenethyl)-2-isobutyl-5-(5-methyl-1,3,4-oxadiazol-2-yl)nicotinamide (50 mg, 0.0921 mmol), KOAc (27 mg, 0.276 mmol) and Pd(dppf)Cl₂ (7 mg, 0.00921 mmol) in EtOH (3 mL) was stirred at 70° C. for 16 hrs under CO. The reaction mixture was cooled down to room temperature, concentrated in vacuo and purified by silica gel column chromatography eluting with 25% EtOAc in petroleum ether to give ethyl 2-(3-carbamoyl-6-(4-fluorophenethyl)-2-isobutyl-5-(5-methyl-1,3,4-oxadiazol-2-yl)pyridin-4-yl)thiazole-5-carboxylate (20 mg, 40.4%). MS: m/z 538.2 (M+H)⁺.

### Step D 2-(3-carbamoyl-6-(4-fluorophenethyl)-2-isobutyl-5-(5-methyl-1,3,4-oxadiazol-2-yl)pyridin-4-yl)thiazole-5-carboxylic acid

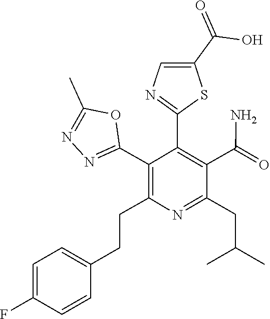 [SMILES: Cc1nnc(-c2c(CCc3ccc(F)cc3)nc(CC(C)C)c(C(N)=O)c2-c2ncc(C(=O)O)s2)o1]

To a solution of ethyl 2-(3-carbamoyl-6-(4-fluorophenethyl)-2-isobutyl-5-(5-methyl-1,3,4-oxadiazol-2-yl)pyridin-4-yl)thiazole-5-carboxylate (20 mg, 0.0372 mmol) in THF (1 mL) and water (0.1 mL) was added LiOH H₂O (8 mg, 0.186 mmol), the mixture was stirred at room temperature for 4 hrs. The reaction mixture was concentrated in vacuo and purified by Prep-HPLC (0.03% TFA) to give 2-(3-carbamoyl-6-(4-fluorophenethyl)-2-isobutyl-5-(5-methyl-1,3,4-oxadiazol-2-yl)pyridin-4-yl)thiazole-5-carboxylic acid (14 mg, 74.1%). MS: m/z 510.2 (M+H)⁺.

### N-(3,4-difluorophenyl)methyl-2-{5-carbamoyl-2-[2-(p-fluorophenyl)ethyl]-6-isobutyl-3-(5-methyl-1,3,4-oxadiazol-2-yl)-4-pyridyl}-1,3-thiazole-5-carboxamide (Compound 125)

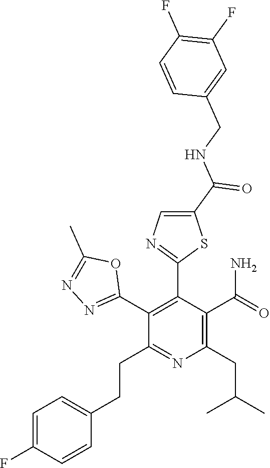 [SMILES: Cc1nnc(-c2c(CCc3ccc(F)cc3)nc(CC(C)C)c(C(N)=O)c2-c2ncc(C(=O)NCc3ccc(F)c(F)c3)s2)o1]

Compound 125 was then synthesized using a similar procedure described in the Example 5 above by using the appropriate materials.

¹H NMR (400 MHz, DMSO-d₆) δ 9.33 (t, J=5.6 Hz, 1H), 8.30 (s, 1H), 8.12 (br s, 1H), 7.78 (br s, 1H), 7.34-7.45 (m, 2H), 7.01-7.20 (m, 5H), 4.44 (d, J=6.0 Hz, 2H), 2.93-3.12 (m, 4H), 2.77 (d, J=7.2 Hz, 2H), 2.39 (s, 3H), 2.26-2.33 (m, 1H), 0.93 (d, J=6.4 Hz, 6H). ¹⁹F NMR (377 MHz, DMSO-d₆) δ −117.26, −138.76, −141.17. MS: m/z 635.2 (M+H)⁺.

### Example 7

### 4-(p-{[(3,4-difluorophenyl)methyl]carbamoyl}phenyl)-6-[2-(p-fluorophenyl)ethyl]-2-isobutyl-5-(5-methyl-1,3,4-oxadiazol-2-yl)nicotinamide (Compound 126)

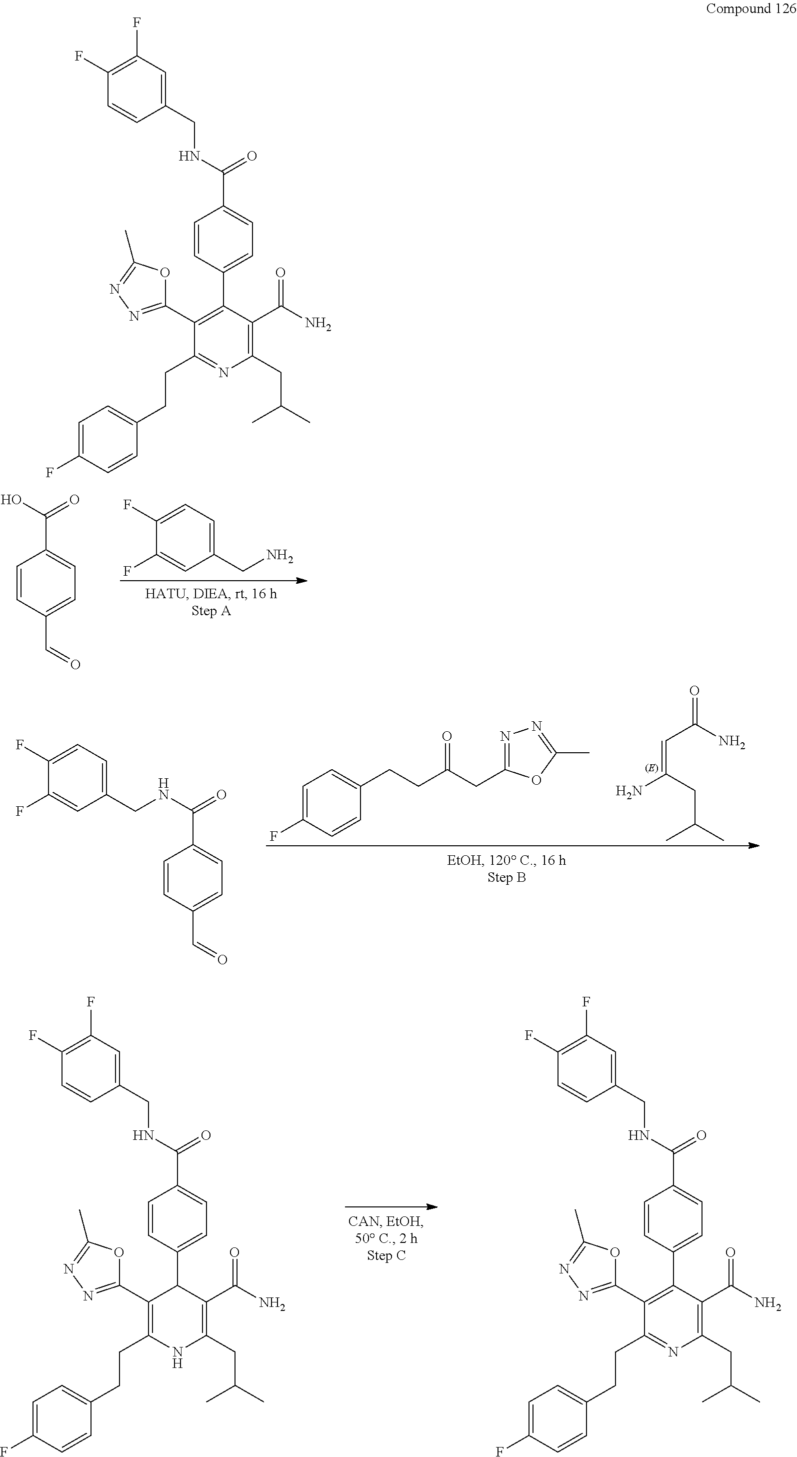

### Step A N-(3,4-difluorobenzyl)-4-formylbenzamide

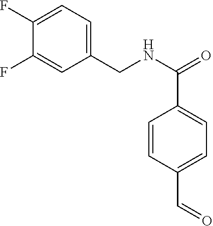 [SMILES: O=Cc1ccc(C(=O)NCc2ccc(F)c(F)c2)cc1]

To a solution of 4-formylbenzoic acid (500 mg, 3.33 mmol) in DMF (1 mL) were added (3,4-difluorophenyl)methanamine (476 mg, 3.33 mmol), HATU (1.52 g, 3.99 mmol) and DIEA (1.29 g, 9.99 mmol) and the reaction mixture was stirred at room temperature for 16 hrs. The reaction mixture was diluted with water (20 mL), extracted with EtOAc (2 mL×5). The organic layers were combined, dried over Na₂SO₄, filtered and concentrated under vacuum to dryness. The crude product was purified by silica gel column chromatography by using 0-10% EtOAc/hexane to give N-(3,4-difluorobenzyl)-4-formylbenzamide (650 mg, 70.91%). ¹H NMR (400 MHz, CDCl₃) δ 10.08 (s, 1H), 7.95 (m, 4H), 6.99-7.26 (m, 3H), 6.57-6.74 (m, 1H), 4.61 (d, J=5.6 Hz, 2H).

### Step B 4-(4-((3,4-difluorobenzyl)carbamoyl)phenyl)-6-(4-fluorophenethyl)-2-isobutyl-5-(5-methyl-1,3,4-oxadiazol-2-yl)-1,4-dihydropyridine-3-carboxamide

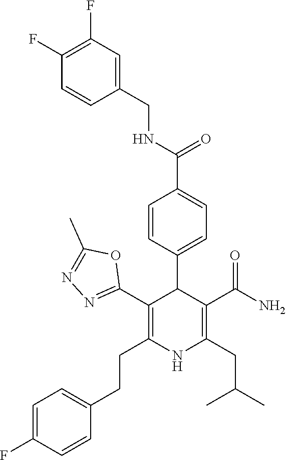 [SMILES: Cc1nnc(C2=C(CCc3ccc(F)cc3)NC(CC(C)C)=C(C(N)=O)C2c2ccc(C(=O)NCc3ccc(F)c(F)c3)cc2)o1]

To a solution of N-(3,4-difluorobenzyl)-4-formylbenzamide (50 mg, 0.18 mmol) in EtOH (1 mL) were added 4-(4-fluorophenyl)-1-(5-methyl-1,3,4-oxadiazol-2-yl)butan-2-one (40 mg, 0.16 mmol) and (E)-3-amino-5-methylhex-2-enamide (23 mg, 0.16 mmol), the reaction mixture was stirred at 110° C. for 16 hrs. The reaction mixture was cooled down to room temperature, diluted with water (10 mL), extracted with EtOAc (2 mL×3). The organic layers were combined, dried over Na₂SO₄, filtered and concentrated under vacuum to dryness. The crude product was purified by Prep-TLC using EtOAc/hexane (1/5) to give 4-(4-((3,4-difluorobenzyl)carbamoyl)phenyl)-6-(4-fluorophenethyl)-2-isobutyl-5-(5-methyl-1,3,4-oxadiazol-2-yl)-1,4-dihydropyridine-3-carboxamide (50 mg, crude). LC-MS: m/z 630.2 (M+H)⁺.

### Step C 4-(p-{[(3,4-difluorophenyl)methyl]carbamoyl}phenyl)-6-[2-(p-fluorophenyl)ethyl]-2-isobutyl-5-(5-methyl-1,3,4-oxadiazol-2-yl)nicotinamide (Compound 126)

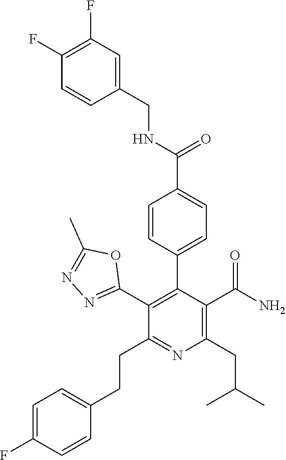 [SMILES: Cc1nnc(-c2c(CCc3ccc(F)cc3)nc(CC(C)C)c(C(N)=O)c2-c2ccc(C(=O)NCc3ccc(F)c(F)c3)cc2)o1]

To a solution of 4-(4-((3,4-difluorobenzyl)carbamoyl)phenyl)-6-(4-fluorophenethyl)-2-isobutyl-5-(5-methyl-1,3,4-oxadiazol-2-yl)-1,4-dihydropyridine-3-carboxamide (50 mg, crude) in EtOH (1 mL) was added diammonium cerium(IV) nitrate (170 mg, 0.31 mmol), and the reaction mixture was stirred at 50° C. for 2 hrs. The reaction mixture was cooled down to room temperature, concentrated in vacuo and purified by Prep-HPLC (0.03% NH₃H₂O) to give 4-(p-{[(3,4-difluorophenyl)methyl]carbamoyl}phenyl)-6-[2-(p-fluorophenyl)ethyl]-2-isobutyl-5-(5-methyl-1,3,4-oxadiazol-2-yl)nicotinamide (19.84 mg, 39.81%).

¹H NMR (400 MHz, DMSO-d₆) δ 9.10 (t, J=4.8 Hz, 1H), 7.74-7.79 (m, 3H), 7.30-7.50 (m, 3H), 6.97-7.24 (m, 7H), 4.43 (d, J=5.2 Hz, 2H), 2.91-3.02 (m, 4H), 2.74 (d, J=6.8 Hz, 2H), 2.24-2.30 (m, 4H), 0.95 (d, J=6.8 Hz, 6H). ¹⁹F NMR (377 MHz, DMSO-d₆) δ −117.30, −138.96, −141.56. LC-MS: m/z 628.1 (M+H)⁺.

### 4-(1-benzothiophen-2-yl)-6-[2-(p-fluorophenyl)ethyl]-2-isobutyl-5-(5-methyl-1,3,4-oxadiazol-2-yl)nicotinamide (Compound 127)

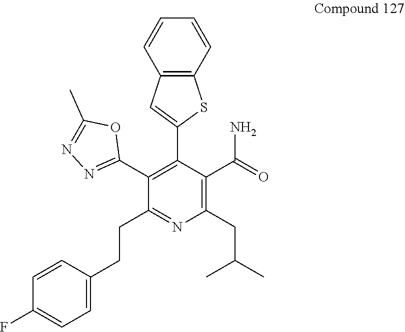 [SMILES: Cc1nnc(-c2c(CCc3ccc(F)cc3)nc(CC(C)C)c(C(N)=O)c2-c2cc3ccccc3s2)o1]

Compound 127 was synthesized using a similar procedure described in the Example 7 above by using the appropriate materials.

¹H NMR (400 MHz, DMSO-d₆) δ 7.91-7.93 (m, 2H), 7.84-7.86 (m, 1H), 7.56 (s, 1H), 7.36-7.39 (m, 3H), 7.10-7.14 (m, 2H), 7.03-7.08 (m, 2H), 2.95-3.03 (m, 4H), 2.75 (d, J=7.2 Hz, 2H), 3.32-3.35 (m, 1H), 2.29 (s, 3H), 0.95 (d, J=6.8 Hz, 6H). ¹⁹F NMR (377 MHz, DMSO-d₆) δ −117.27. LC-MS: m/z 514.9 (M+H)⁺.

### N-(3,4-difluorophenyl)methyl-5-{5-carbamoyl-2-[2-(p-fluorophenyl)ethyl]-6-isobutyl-3-(5-methyl-1,3,4-oxadiazol-2-yl)-4-pyridyl}-2-furamide (Compound 128)

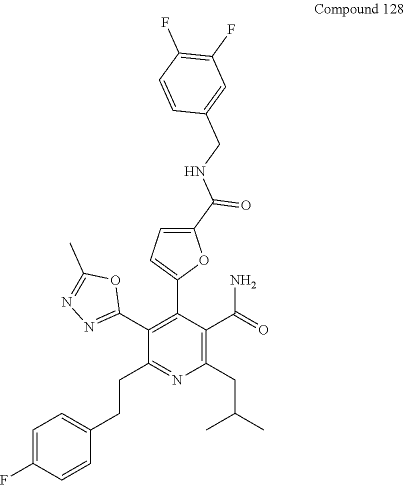 [SMILES: Cc1nnc(-c2c(CCc3ccc(F)cc3)nc(CC(C)C)c(C(N)=O)c2-c2ccc(C(=O)NCc3ccc(F)c(F)c3)o2)o1]

Compound 128 was synthesized using a similar procedure described in the Example 7 above by using the appropriate materials.

¹H NMR (400 MHz, DMSO-d₆) δ 8.65 (t, J=6.4 Hz, 1H), 8.05 (br s, 1H), 7.78 (br s, 1H), 7.27-7.44 (m, 2H), 7.19 (d, J=3.6 Hz, 1H), 7.01-7.15 (m, 5H), 6.82 (d, J=3.6 Hz, 1H), 4.35 (d, J=6.0 Hz, 2H), 2.92-3.05 (m, 4H), 2.73 (d, J=7.2 Hz, 2H), 2.39 (s, 3H), 2.25-2.33 (m, 1H), 0.91 (d, J=6.4 Hz, 6H). ¹⁹F NMR (377 MHz, DMSO-d₆) δ −117.30, −138.89, −141.39. LC-MS: m/z 618.1 (M+H)⁺.

### Example 8

### 4-(7-bromo-1-benzothiophen-2-yl)-6-[2-(p-fluorophenyl)ethyl]-2-isobutyl-5-(5-methyl-1,3,4-oxadiazol-2-yl)nicotinamide (Compound 129)

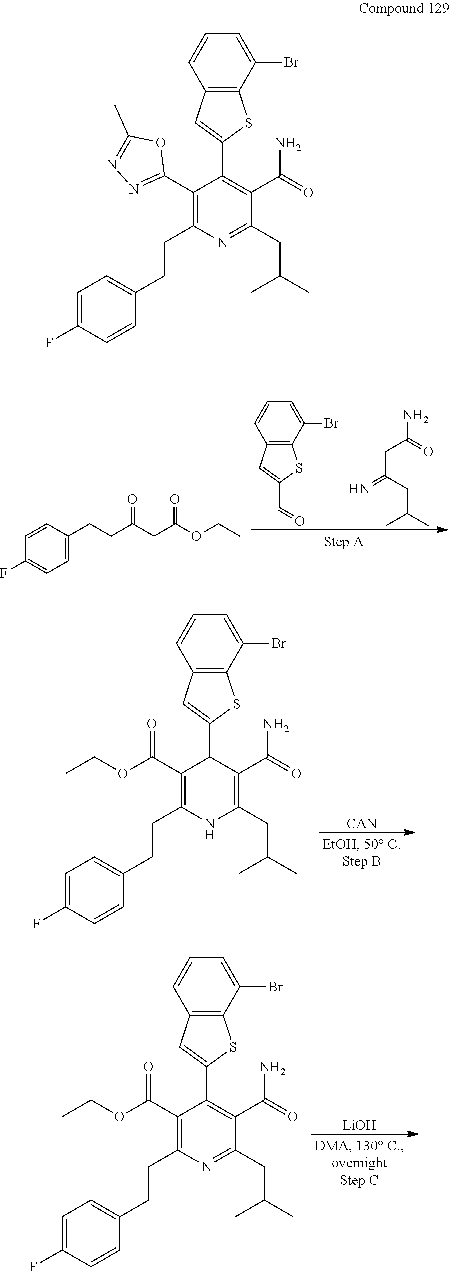

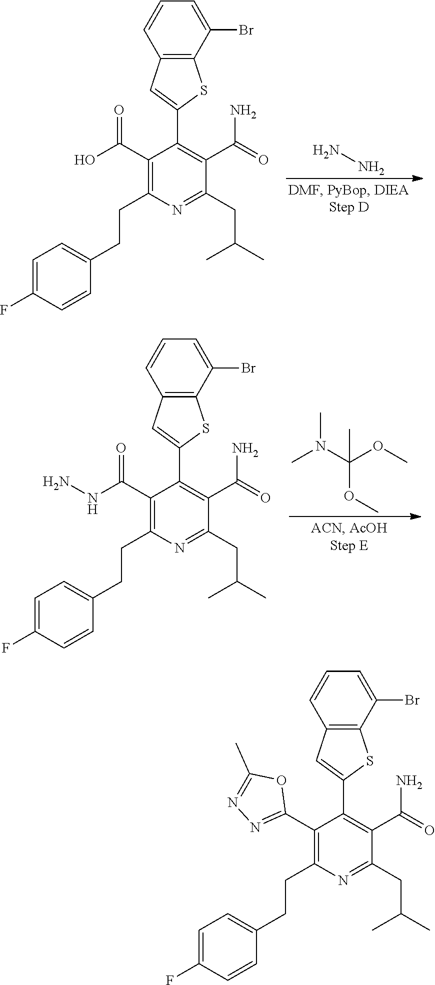

### Step A ethyl 4-(7-bromobenzo[b]thiophen-2-yl)-5-carbamoyl-2-(4-fluorophenethyl)-6-isobutyl-1,4-dihydropyridine-3-carboxylate

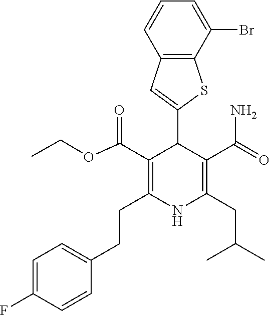 [SMILES: CCOC(=O)C1=C(CCc2ccc(F)cc2)NC(CC(C)C)=C(C(N)=O)C1c1cc2cccc(Br)c2s1]

A solution of ethyl 5-(4-fluorophenyl)-3-oxopentanoate (500 mg, 2.099 mmol), 7-bromobenzo[b]thiophene-2-carbaldehyde (505.96 mg, 2.099 mmol), 3-imino-5-methylhexanamide (298.41 mg, 2.099 mmol) in EtOH (10.0 mL) was stirred at 120° C. overnight. The reaction mixture was cooled down to room temperature, concentrated in vacuum to give ethyl 4-(7-bromobenzo[b]thiophen-2-yl)-5-carbamoyl-2-(4-fluorophenethyl)-6-isobutyl-1,4-dihydropyridine-3-carboxylate (1.23 g, crude). LC-MS: m/z 584.9 (M+H)⁺.

### Step B ethyl 4-(7-bromobenzo[b]thiophen-2-yl)-5-carbamoyl-2-(4-fluorophenethyl)-6-isobutylnicotinate

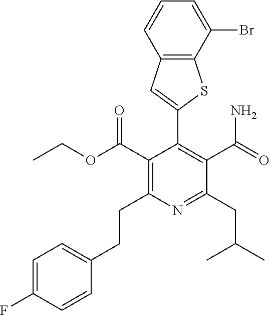 [SMILES: CCOC(=O)c1c(CCc2ccc(F)cc2)nc(CC(C)C)c(C(N)=O)c1-c1cc2cccc(Br)c2s1]

To a mixture of ethyl 4-(7-bromobenzo[b]thiophen-2-yl)-5-carbamoyl-2-(4-fluorophenethyl)-6-isobutyl-1,4-dihydropyridine-3-carboxylate (1.23 g, crude) in EtOH (10.0 mL) was added Diammonium cerium(IV)nitrate (1.73 g, 3.151 mmol) at room temperature. The mixture was stirred at 50° C. for 1 hr. A The reaction mixture was cooled down to room temperature, quenched with H₂O (200 mL) and extracted with EtOAc (100 mL×3). The organic layer was combined and washed with brine (100 mL×2), dried over Na₂SO₄, filtered and concentrated in vacuum. The reaction mixture was purified by column chromatography (PE/EA=3/1) to give ethyl 4-(7-bromobenzo[b]thiophen-2-yl)-5-carbamoyl-2-(4-fluorophenethyl)-6-isobutylnicotinate (800 mg, 65.26%). LC-MS: m/z 582.9 (M+H)⁺.

### Step C 4-(7-bromobenzo[b]thiophen-2-yl)-5-carbamoyl-2-(4-fluorophenethyl)-6-isobutylnicotinic acid

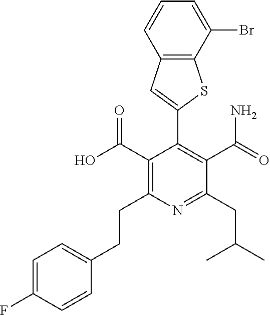 [SMILES: CC(C)Cc1nc(CCc2ccc(F)cc2)c(C(=O)O)c(-c2cc3cccc(Br)c3s2)c1C(N)=O]

To a solution of ethyl 4-(7-bromobenzo[b]thiophen-2-yl)-5-carbamoyl-2-(4-fluorophenethyl)-6-isobutylnicotinate (540 mg, 0.925 mmol) in DMA (10.0 mL) was added LiCl (392.28 mg, 9.25 mmol) at room temperature. The mixture was stirred at 130° C. overnight. The reaction mixture was cooled down to room temperature, concentrated and purified by reversed phase column (HCOOH, 0.1%) to give 4-(7-bromobenzo[b]thiophen-2-yl)-5-carbamoyl-2-(4-fluorophenethyl)-6-isobutylnicotinic acid (140 mg, 27.24%). LC-MS: m/z 554.8 (M+H)⁺.

### Step D The synthesis of 4-(7-bromobenzo[b]thiophen-2-yl)-6-(4-fluorophenethyl)-5-(hydrazinecarbonyl)-2-isobutylnicotinamide

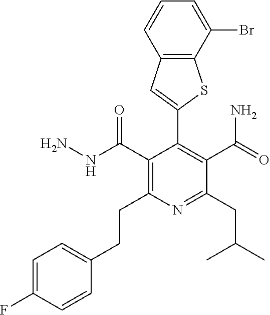 [SMILES: CC(C)Cc1nc(CCc2ccc(F)cc2)c(C(=O)NN)c(-c2cc3cccc(Br)c3s2)c1C(N)=O]

To a mixture of 4-(7-bromobenzo[b]thiophen-2-yl)-5-carbamoyl-2-(4-fluorophenethyl)-6-isobutylnicotinic acid (140 mg, 0.252 mmol), Hydrazine monohydrochloride (25.90 mg, 0.378 mmol) in DMF (5.0 mL) was added PyBOP (393.19 mg, 0.756 mmol) and DIEA (0.3 mL) at room temperature. The mixture was stirred at room temperature for 1 hour. After the reaction was completed, the reaction was quenched with H₂O (20 mL) and extracted with ethyl acetate (20 mL×3). The organic layer was combined and washed with brine (20 mL×2), dried over sodium sulfate, filtered and concentrated in vacuum. The reaction was purified by column chromatography (DCM/MeOH=20/1) to give 4-(7-bromobenzo[b]thiophen-2-yl)-6-(4-fluorophenethyl)-5-(hydrazinecarbonyl)-2-isobutylnicotinamide (90 mg, yield: 62.70%). LC-MS: m/z 569.1 (M+H)⁺.

### Step E 4-(7-bromo-1-benzothiophen-2-yl)-6-[2-(p-fluorophenyl)ethyl]-2-isobutyl-5-(5-methyl-1,3,4-oxadiazol-2-yl)nicotinamide (Compound 129)

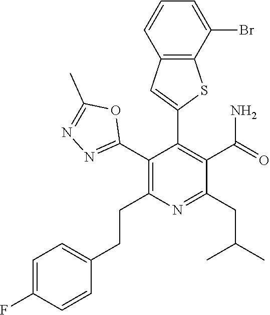 [SMILES: Cc1nnc(-c2c(CCc3ccc(F)cc3)nc(CC(C)C)c(C(N)=O)c2-c2cc3cccc(Br)c3s2)o1]

To a solution of 4-(7-bromobenzo[b]thiophen-2-yl)-6-(4-fluorophenethyl)-5-(hydrazinecarbonyl)-2-isobutylnicotinamide (80 mg, 0.140 mmol) in MeCN (8.0 mL) was added 1,1-dimethoxy-N,N-dimethylethan-1-amine (28.07 mg, 0.211 mmol) at room temperature. The solution was stirred at 80° C. for 1 h. HoAc (0.5 mL) was added. The solution was stirred at 100° C. overnight. After the reaction was completed, the reaction was concentrated in vacuum. The reaction was purified by Prep-HPLC to give Compound 129 (44.84 mg, yield: 53.78%). ¹H NMR (400 MHz, DMSO-d₆) δ 7.97 (s, 1H), 7.90 (d, J=24.8 Hz, 1H), 7.64 (d, J=21.2 Hz, 1H), 7.58 (s, 1H), 7.50 (s, 1H), 7.36 (t, J=7.6 Hz, 1H), 7.01-7.16 (m, 4H), 2.93-3.06 (m, 4H), 2.76 (d, J=7.6 Hz, 2H), 2.27-2.35 (m, 4H), 0.95 (d, J=6.8 Hz, 6H). ¹⁹F NMR (377 MHz, DMSO-d₆) δ −117.26. LC-MS: m/z 593.0 (M+H)⁺.

### Example 9

### 4-(7-{[(3,4-difluorophenyl)methyl]amino}-1-oxa-6-aza-2-indenyl)-6-[2-(p-fluorophenyl)ethyl]-2-isobutyl-5-(5-methyl-1,3,4-oxadiazol-2-yl)nicotinamide (Compound 130)

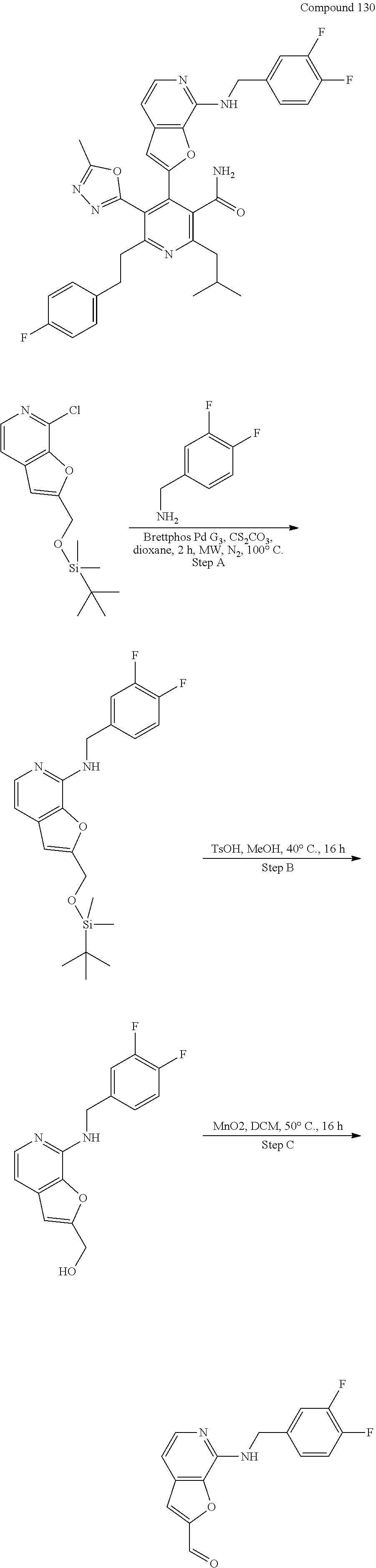

### Step A 2-(((tert-butyldimethylsilyl)oxy)methyl)-N-(3,4-difluorobenzyl)furo[2,3-c]pyridin-7-amine

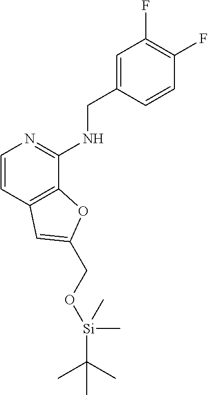 [SMILES: CC(C)(C)[Si](C)(C)OCc1cc2ccnc(NCc3ccc(F)c(F)c3)c2o1]

To a solution of 2-(((tert-butyldimethylsilyl)oxy)methyl)-7-chlorofuro[2,3-c]pyridine (250 mg, 1.09 mmol) and (3,4-difluorophenyl)methanamine (310 mg, 2.17 mmol) in dioxane (10 mL) were added Brettphos Pd G₃ (99 mg, 0.11 mmol) and Cs₂CO₃ (1.06 g, 3.27 mmol), and the mixture was stirred at 100° C. for 2 hrs under N₂ in microwave. The reaction mixture was cooled down to room temperature, concentrated and the mixture was purified by column chromatography on silica gel (EtOAc: PE=0 to 20%) to give 2-(((tert-butyldimethylsilyl)oxy)methyl)-N-(3,4-difluorobenzyl)furo[2,3-c]pyridin-7-amine (175 mg, 40.0%). LC-MS: m/z 405.1 (M+H)⁺.

### Step B (7-((3,4-difluorobenzyl)amino)furo[2,3-c]pyridin-2-yl)methanol

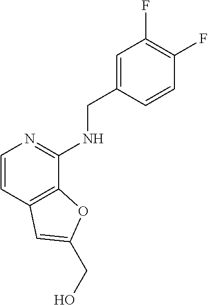 [SMILES: OCc1cc2ccnc(NCc3ccc(F)c(F)c3)c2o1]

To a solution of 2-(((tert-butyldimethylsilyl)oxy)methyl)-N-(3,4-difluorobenzyl)furo[2,3-c]pyridin-7-amine (175 mg, 0.43 mmol) in MeOH (15 mL) was added TsOH (164 mg, 0.87 mmol), the mixture was stirred at 40° C. for 16 hrs. The reaction mixture was cooled down to room temperature, concentrated and the mixture was purified by reserve flash (0.1 FA/H₂O: ACN=0 to 10%) to give (7-((3,4-difluorobenzyl)amino)furo[2,3-c]pyridin-2-yl)methanol (100 mg, 79.4%). LC-MS: m/z 290.9 (M+H)⁺.

### Step C 4-(7-{[(3,4-difluorophenyl)methyl]amino}-1-oxa-6-aza-2-indenyl)-6-[2-(p-fluorophenyl)ethyl]-2-isobutyl-5-(5-methyl-1,3,4-oxadiazol-2-yl)nicotinamide

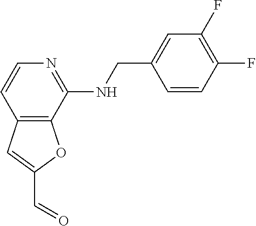 [SMILES: O=Cc1cc2ccnc(NCc3ccc(F)c(F)c3)c2o1]

To a solution of (7-((3,4-difluorobenzyl)amino)furo[2,3-c]pyridin-2-yl)methanol (100 mg, 0.10 mmol) in DCM (10 mL) was added MnO₂ (260 mg, 1.00 mmol), the mixture was stirred at 50° C. for 16 hours. The reaction mixture was cooled down to room temperature, filtered and the filtrate was concentrated to give 7-((3,4-difluorobenzyl)amino)furo[2,3-c]pyridine-2-carbaldehyde (50 mg, crude, 50.5%). LC-MS: m/z 289.1 (M+H)⁺.

### 4-(7-{[(3,4-difluorophenyl)methyl]amino}-1-oxa-6-aza-2-indenyl)-6-[2-(p-fluorophenyl)ethyl]-2-isobutyl-5-(5-methyl-1,3,4-oxadiazol-2-yl)nicotinamide (Compound 130)

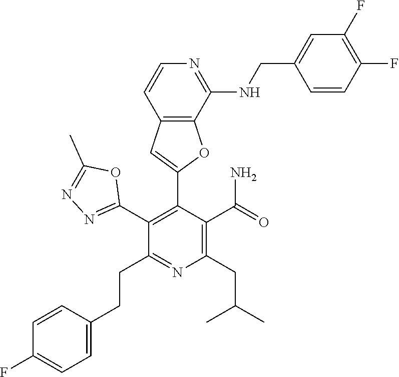 [SMILES: Cc1nnc(-c2c(CCc3ccc(F)cc3)nc(CC(C)C)c(C(N)=O)c2-c2cc3ccnc(NCc4ccc(F)c(F)c4)c3o2)o1]

Compound 130 was then synthesized using a similar procedure described in the Example 7 above by using the appropriate materials.

¹H NMR (400 MHz, DMSO-d₆) δ 8.11 (s, 1H), 7.82 (s, 1H), 7.73 (d, J=5.2 Hz, 1H), 7.29-7.39 (m, 2H), 7.13-7.17 (m, 3H), 7.04-7.08 (m, 3H), 6.87-6.93 (m, 2H), 4.57 (d, J=5.6 Hz, 2H), 3.08-3.11 (m, 2H), 2.98-3.02 (m, 2H), 2.77 (d, J=6.8 Hz, 2H), 2.46-2.48 (m, 1H), 2.30 (s, 3H), 0.93 (d, J=6.8 Hz, 6H). ¹⁹F NMR (377 MHz, DMSO-d₆) δ −117.25, −139.27, −142.07. LC-MS: m/z 641.3 (M+H)⁺.

### Example 10

### 4-(7-{[(3,4-difluorophenyl)methyl]amino}-1-thia-6-aza-2-indenyl)-6-[2-(p-fluorophenyl)ethyl]-2-isobutyl-5-(5-methyl-1,3,4-oxadiazol-2-yl)nicotinamide (Compound 131)

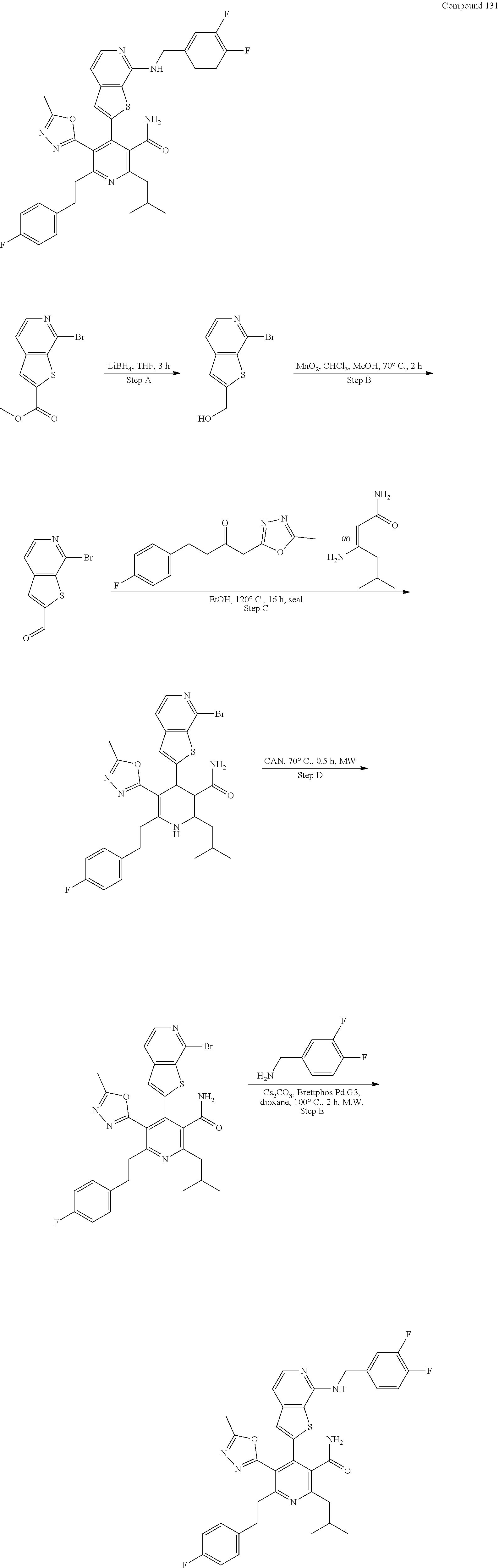

### Step A (7-bromothieno[2,3-c]pyridin-2-yl)methanol

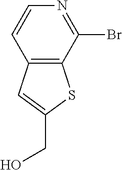 [SMILES: OCc1cc2ccnc(Br)c2s1]

To a mixture of methyl 7-bromothieno[2, 3-c]pyridine-2-carboxylate (900 mg, 3.31 mmol) in THF (10 mL) was added LiBH₄ (288.14 mg, 13.23 mmol) at 0° C. and the reaction mixture was stirred at room temperature for 1 hr. The reaction mixture was diluted with EtOAc and brine. The organic layer was separated, washed with brine, dried over Na₂SO₄, filtered and concentrated in vacuo to give (7-bromothieno[2,3-c]pyridin-2-yl)methanol (440 mg, 54.50%). LC-MS: m/z 243.9 (M+H)⁺.

### Step B 7-bromothieno[2,3-c]pyridine-2-carbaldehyde

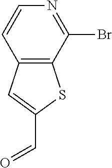 [SMILES: O=Cc1cc2ccnc(Br)c2s1]

To a mixture of (7-bromothieno[2,3-c]pyridin-2-yl)methanol (400 mg, 1.64 mmol) in CHCl₃ (10 mL) and MeOH (1 mL), was added MnO₂ (2.85 g, 32.77 mmol) and the reaction mixture was stirred at 70° C. for 6 hrs. The reaction mixture was filtered and concentrated in vacuo. The residue was purified by silica gel column chromatography eluting with PE:EtOAc=2:1 to give 7-bromothieno[2,3-c]pyridine-2-carbaldehyde (340 mg, 85.71%). LC-MS: m/z 241.9 (M+H)⁺.

### Step C 4-(7-bromothieno[2,3-c]pyridin-2-yl)-6-[2-(4-fluorophenyl)ethyl]-5-(5-methyl-1,3,4-oxadiazol-2-yl)-2-(2-methylpropyl)-1,4-dihydropyridine-3-carboxamide

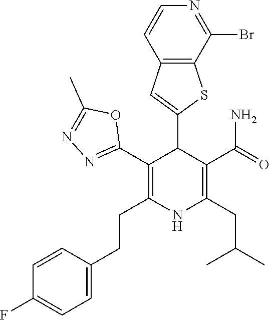 [SMILES: Cc1nnc(C2=C(CCc3ccc(F)cc3)NC(CC(C)C)=C(C(N)=O)C2c2cc3ccnc(Br)c3s2)o1]

To a mixture of 7-bromothieno[2,3-c]pyridine-2-carbaldehyde (320 mg, 1.32 mmol) in EtOH (15 mL) were added 4-(4-fluorophenyl)-1-(5-methyl-1,3,4-oxadiazol-2-yl) butan-2-one (329 mg, 1.32 mmol) and (2E)-3-amino-5-methylhex-2-enamide (188 mg, 1.32 mmol). The reaction mixture was stirred at 120° C. in a seal tube with N₂ protection for overnight. The residue was purified by silica gel column chromatography eluting with DCM:MeOH=20:1 to give 4-(7-bromothieno[2,3-c]pyridin-2-yl)-6-[2-(4-fluorophenyl)ethyl]-5-(5-methyl-1,3,4-oxadiazol-2-yl)-2-(2-methylpropyl)-1,4-dihydropyridine-3-carboxamide (580 mg, 73.56%). LC-MS: m/z 596.1 (M+H)⁺.

### Step D 4-(7-bromothieno[2,3-c]pyridin-2-yl)-6-[2-(4-fluorophenyl)ethyl]-5-(5-methyl-1,3,4-oxadiazol-2-yl)-2-(2-methylpropyl)pyridine-3-carboxamide

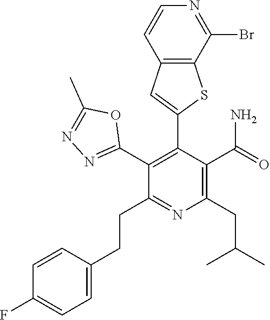 [SMILES: Cc1nnc(-c2c(CCc3ccc(F)cc3)nc(CC(C)C)c(C(N)=O)c2-c2cc3ccnc(Br)c3s2)o1]

To a mixture of 4-(7-bromothieno[2,3-c]pyridin-2-yl)-6-[2-(4-fluorophenyl) ethyl]-5-(5-methyl-1,3,4-oxadiazol-2-yl)-2-(2-methylpropyl)-1,4-dihydropyridine-3-carboxamide (80 mg, 0.134 mmol) in DCM (4 mL) were added ceric ammonium nitrate (221 mg, 0.402 mmol) and the reaction mixture was stirred at 70° C. for 30 min under microwave. The reaction mixture was cooled down to room temperature, filtered and concentrated in vacuo to give 4-(7-bromothieno[2,3-c]pyridin-2-yl)-6-[2-(4-fluorophenyl)ethyl]-5-(5-methyl-1,3,4-oxadiazol-2-yl)-2-(2-methylpropyl)pyridine-3-carboxamide (50 mg, 62.71%). LC-MS: m/z 594.1 (M+H)⁺.

### Step E 4-(7-{[(3,4-difluorophenyl)methyl]amino}-1-thia-6-aza-2-indenyl)-6-[2-(p-fluorophenyl)ethyl]-2-isobutyl-5-(5-methyl-1,3,4-oxadiazol-2-yl)nicotinamide (Compound 131)

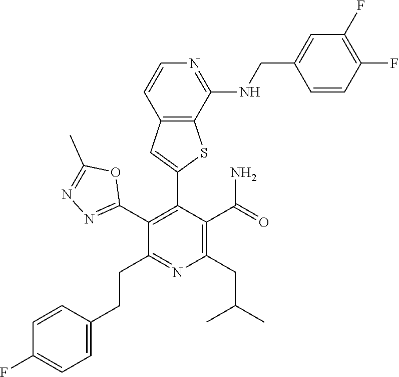 [SMILES: Cc1nnc(-c2c(CCc3ccc(F)cc3)nc(CC(C)C)c(C(N)=O)c2-c2cc3ccnc(NCc4ccc(F)c(F)c4)c3s2)o1]

To a mixture of 4-(7-bromothieno[2,3-c]pyridin-2-yl)-6-[2-(4-fluorophenyl)ethyl]-5-(5-methyl-1,3,4-oxadiazol-2-yl)-2-(2-methylpropyl) pyridine-3-carboxamide (50 mg, 0.084 mmol) in dioxane (4 mL) were added (3,4-difluorophenyl)methanamine (24 mg, 0.17 mmol), Cs₂CO₃ (55 mg, 0.17 mmol), and BrettPhos Pd G3 (8 mg, 0.008 mmol). The reaction mixture was stirred at 100° C. for 2 hrs under microwave with N₂ protection. The reaction mixture was cooled down to room temperature, filtered and concentrated in vacuo. The residue was purified by Pre-HPLC with 0.1% FA to give 4-(7-{[(3,4-difluorophenyl)methyl]amino}-1-oxa-6-aza-2-indenyl)-6-[2-(p-fluorophenyl)ethyl]-2-isobutyl-5-(5-methyl-1,3,4-oxadiazol-2-yl)nicotinamide (4.1 mg, 7.42%).

¹H NMR (400 MHz, CD₃OD) δ 7.87 (d, J=6.0 Hz, 1H), 7.30 (s, 1H), 7.13-7.25 (m, 3H), 7.02-7.06 (m, 3H), 6.92-6.96 (m, 2H), 4.69 (s, 2H), 3.15-3.19 (m, 2H), 3.02-3.07 (m, 2H), 2.90 (d, J=7.2 Hz, 2H), 2.38-2.45 (m, 1H), 2.29 (s, 3H), 1.04 (d, J=6.8 Hz, 6H). ¹⁹F NMR (377 MHz, CD₃OD) δ −119.31, −141.11, −143.93. LC-MS: m/z 651.3 (M+H)⁺.

### 4-(4-((3,4-difluorobenzyl)amino)thieno[3,2-d]pyrimidin-6-yl)-6-(4-fluorophenethyl)-2-isobutyl-5-(5-methyl-1,3,4-oxadiazol-2-yl)nicotinamide (Compound 132)

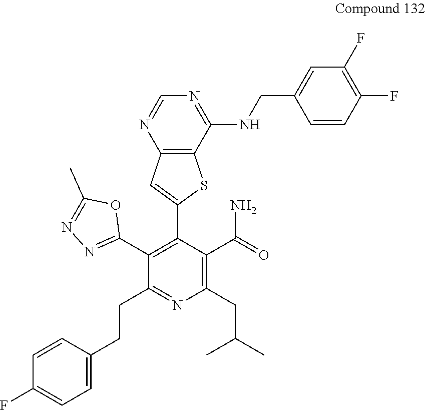 [SMILES: Cc1nnc(-c2c(CCc3ccc(F)cc3)nc(CC(C)C)c(C(N)=O)c2-c2cc3ncnc(NCc4ccc(F)c(F)c4)c3s2)o1]

Compound 132 was synthesized using a similar procedure described in the Example 10 above by using the appropriate materials.

¹H NMR (400 MHz, CD₃OD) δ 8.44 (s, 1H), 7.30 (s, 1H), 7.19-7.28 (m, 3H), 7.03-7.07 (m, 2H), 6.92-6.97 (m, 2H), 4.76 (s, 2H), 3.14-3.18 (m, 2H), 3.04-3.08 (m, 2H), 2.90 (d, J=7.2 Hz, 2H), 2.38-2.45 (m, 1H), 2.33 (s, 3H), 1.04 (d, J=6.8 Hz, 6H). ¹⁹F NMR (377 MHz, CD₃OD) δ −119.30, −140.66, −140.71, −143.12, −143.18. LC-MS: m/z 658.3 (M+H)⁺.

### 4-(4-((3,4-difluorobenzyl)amino)thieno[3,2-c]pyridin-2-yl)-6-(4-fluorophenethyl)-2-isobutyl-5-(5-methyl-1,3,4-oxadiazol-2-yl)nicotinamide (Compound 133)

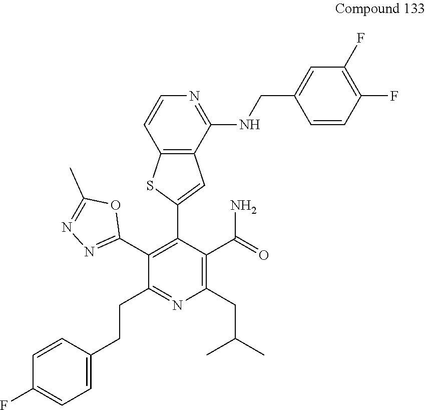 [SMILES: Cc1nnc(-c2c(CCc3ccc(F)cc3)nc(CC(C)C)c(C(N)=O)c2-c2cc3c(NCc4ccc(F)c(F)c4)nccc3s2)o1]

Compound 133 was synthesized using a similar procedure described in the Example 10 above by using the appropriate materials.

¹H NMR (400 MHz, DMSO-d₆) δ 7.87 (s, 1H), 7.79-7.83 (m, 2H), 7.59 (s, 1H), 7.56 (s, 1H), 7.32-7.37 (m, 2H), 7.03-7.14 (m, 6H), 4.62 (d, J=5.6 Hz, 2H), 2.99-3.00 (m, 4H), 2.75 (d, J=7.6 Hz, 2H), 2.32-2.33 (m, 1H), 2.31 (s, 3H), 0.93 (d, J=6.8 Hz, 6H). ¹⁹F NMR (377 MHz, DMSO-d₆) δ −117.26, −139.21, −139.27, −141.98, −142.04. LC-MS: m/z 657.0 (M+H)⁺.

### 6-(4-fluorophenethyl)-2-isobutyl-4-(7-(3-(methoxymethyl)azetidin-1-yl)thieno[2,3-c]pyridin-2-yl)-5-(5-methyl-1,3,4-oxadiazol-2-yl)nicotinamide (Compound 134)

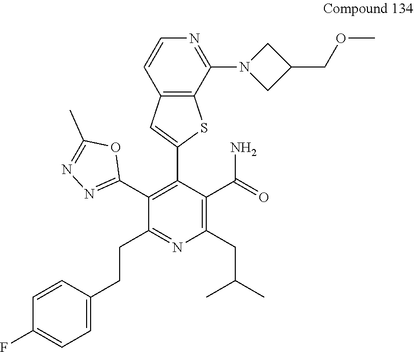 [SMILES: COCC1CN(c2nccc3cc(-c4c(C(N)=O)c(CC(C)C)nc(CCc5ccc(F)cc5)c4-c4nnc(C)o4)sc23)C1]

Compound 134 was synthesized using a similar procedure described in the Example 10 above by using the appropriate materials.

¹H NMR (400 MHz, DMSO-d6): δ 7.89-7.96 (m, 2H), 7.60 (br s, 1H), 7.31 (s, 1H), 7.01-7.17 (m, 5H), 4.17 (t, J=8.0 Hz, 2H), 3.87 (dd, J=6.0 Hz, J=7.6 Hz, 2H), 3.53 (d, J=6.0 Hz, 2H), 3.30 (s, 2H), 3.28 (s, 3H), 2.95-3.02 (m, 4H), 2.75 (d, J=7.2 Hz, 2H), 2.33 (s, 3H), 0.94 (d, J=6.8 Hz, 6H). ¹⁹F NMR (377 MHz, DMSO-d₆) δ −117.26. LC-MS: m/z 615.2 (M+H)⁺.

### Example 11

### 6-(4-fluorophenethyl)-2-isobutyl-4-(7-(3-methoxypiperidin-1-yl)thieno[2,3-c]pyridin-2-yl)-5-(5-methyl-1,3,4-oxadiazol-2-yl)nicotinamide (Compound 135)

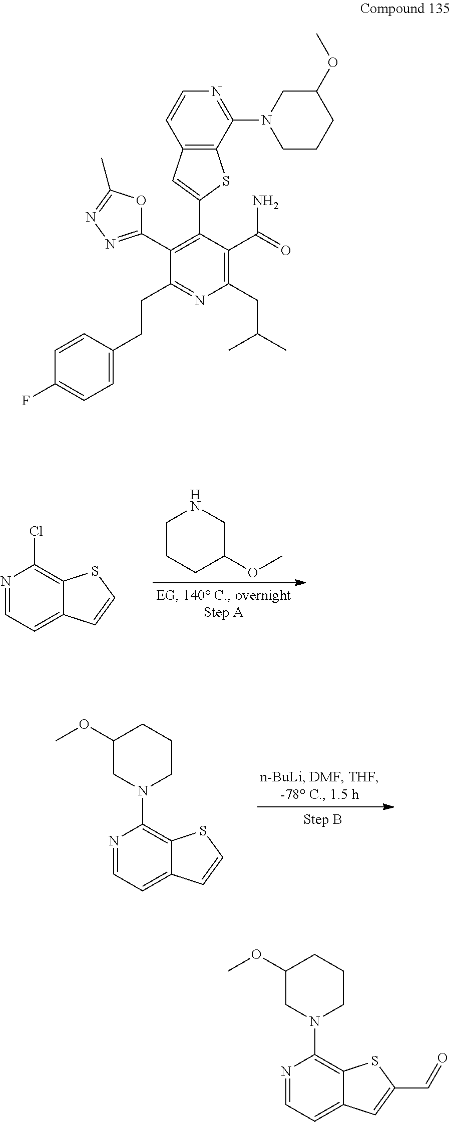

### Step A 7-(3-methoxypiperidin-1-yl)thieno[2,3-c]pyridine

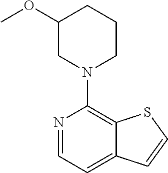 [SMILES: COC1CCCN(c2nccc3ccsc23)C1]

To a solution of 7-chlorothieno[2,3-c]pyridine (950 mg, 5.60 mmol) in ethylene glycol (10 mL) were added 3-methoxyhexahydropyridine (1935.17 mg, 16.80 mmol), the reaction was stirred at 140° C. for overnight. After the reaction was completed, the mixture was concentrated in vacuum. The residue was purified by column chromatography on silica gel (PE/EtOAc=10:1) to give 7-(3-methoxypiperidin-1-yl)thieno[2,3-c]pyridine (1.3 g mg, yield: 93.47%). LC-MS: m/z 249.1 (M+H)⁺.

### Step B 7-(3-methoxypiperidin-1-yl)thieno[2,3-c]pyridine-2-carbaldehyde

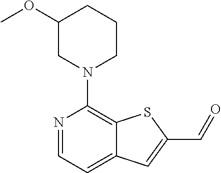 [SMILES: COC1CCCN(c2nccc3cc(C=O)sc23)C1]

To a solution of 7-(3-methoxyhexahydropyridin-1-yl)thieno[2,3-c]pyridine (500 mg, 2.013 mmol) in tetrahydrofuran (10 mL) was added dropwise n-BuLi (1.13 mL, 2.819 mmol, 2.5 M in n-hexane) at −78° C. and stirred at −78° C. for 30 min, then N,N-dimethylmethanamide (0.211 mL, 2.617 mmol) was added. The reaction mixture was gradually warmed to room temperature, stirred for further 1 h. The reaction mixture was diluted with EA and water. The organic layer was separated, washed with further water. Then the organic layer was dried with Na₂SO₄, filtered and concentrated in vacuo. The residue was purified using silica gel column chromatography eluting with ethyl acetate in petroleum ether (PE:EtOAc=9:1). The organic layer was collected, concentrated in vacuo to afford the title compound 7-(3-methoxypiperidin-1-yl)thieno[2,3-c]pyridine-2-carbaldehyde (395 mg, yield: 70.99%). LC-MS: m/z 277.0 (M+H)⁺.

### 6-(4-fluorophenethyl)-2-isobutyl-4-(7-(3-methoxypiperidin-1-yl)thieno[2,3-c]pyridin-2-yl)-5-(5-methyl-1,3,4-oxadiazol-2-yl)nicotinamide (Compound 135)

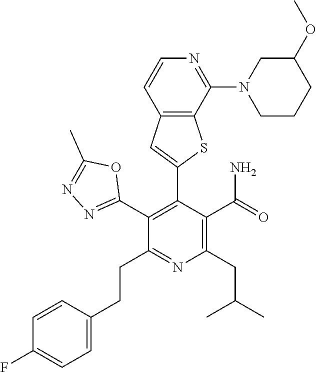 [SMILES: COC1CCCN(c2nccc3cc(-c4c(C(N)=O)c(CC(C)C)nc(CCc5ccc(F)cc5)c4-c4nnc(C)o4)sc23)C1]

Compound 135 was then synthesized using a similar procedure described in the Example 7 above by using 7-(3-methoxypiperidin-1-yl)thieno[2,3-c]pyridine-2-carbaldehyde and the appropriate materials.

¹H NMR (400 MHz, DMSO-d₆) δ 8.04 (d, J=5.6 Hz, 1H), 7.92 (br s, 1H), 7.60 (br s, 1H), 7.35 (s, 1H), 7.25 (d, J=5.6 Hz, 1H), 7.02-7.15 (m, 4H), 3.96-4.05 (m, 1H), 3.70-3.79 (m, 1H), 3.26 (s, 3H), 2.94-3.22 (m, 7H), 2.75 (d, J=7.2 Hz, 2H), 2.27-2.35 (m, 4H), 1.96-2.04 (m, 1H), 1.75-1.85 (m, 1H), 1.41-1.56 (m, 2H), 0.94 (d, J=6.8 Hz, 6H). ¹⁹F NMR (377 MHz, DMSO-d₆) δ −117.27. LC-MS: m/z 629.1 (M+H)⁺.

### Example 12

### 4-(7-((3,4-difluorobenzyl)amino)benzo[b]thiophen-2-yl)-6-(4-fluorophenethyl)-2-isobutyl-5-(5-methyl-1,3,4-oxadiazol-2-yl)nicotinamide (Compound 136)

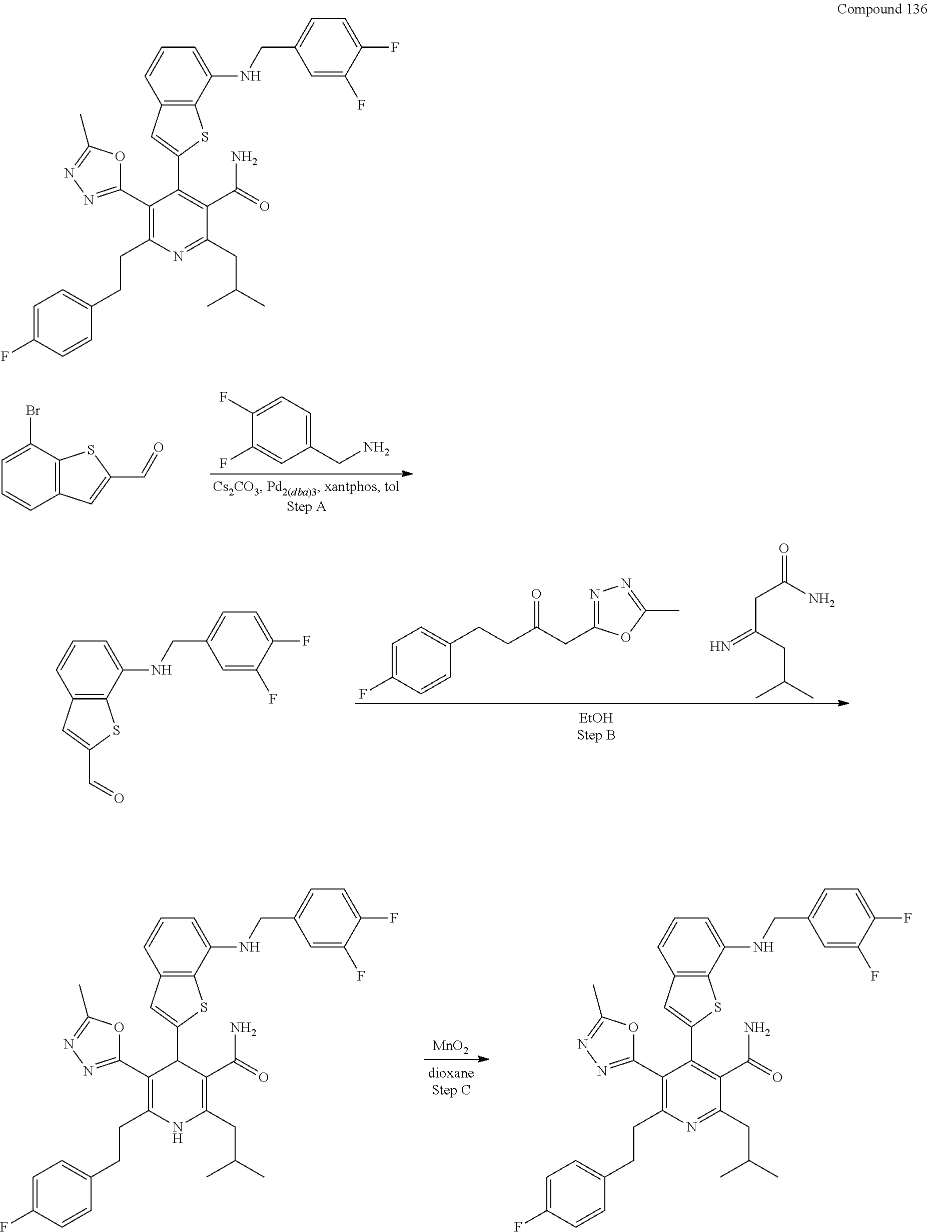

### Step A 7-((3,4-difluorobenzyl)amino)benzo[b]thiophene-2-carbaldehyde

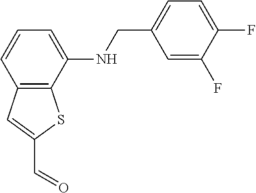 [SMILES: O=Cc1cc2cccc(NCc3ccc(F)c(F)c3)c2s1]

To a mixture of 7-bromobenzo[b]thiophene-2-carbaldehyde (70 mg, 0.290 mmol), (3,4-difluorophenyl)methanamine (49.87 mg, 0.348 mmol), Cs₂CO₃ (283.95 mg, 0.871 mmol) in toluene (3 mL) was added Xantphos (16.75 mg, 0.029 mmol) and Pd₂(dba)₃ (26.57 mg, 0.029 mmol) at rt. The mixture was stirred at 130° C. for 1 h under microwave. After the reaction was completed, the reaction was quenched with H₂O (25 mL) and extracted with ethyl acetate (20 mL×3). The organic layer was combined and washed with brine (20 mL×2), dried over sodium sulfate, filtered and concentrated in vacuum. The mixture was purified by column chromatography (PE/EA=10/1) to give 7-((3,4-difluorobenzyl)amino)benzo[b]thiophene-2-carbaldehyde (40 mg, 0.132 mmol, 45.42%). LC-MS: m/z 304.1 (M+H)⁺.

### Step B 4-(7-((3,4-difluorobenzyl)amino)benzo[b]thiophen-2-yl)-6-(4-fluorophenethyl)-2-isobutyl-5-(5-methyl-1,3,4-oxadiazol-2-yl)-1,4-dihydropyridine-3-carboxamide

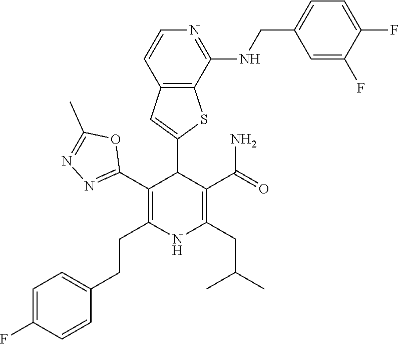 [SMILES: Cc1nnc(C2=C(CCc3ccc(F)cc3)NC(CC(C)C)=C(C(N)=O)C2c2cc3ccnc(NCc4ccc(F)c(F)c4)c3s2)o1]

A mixture of 7-((3,4-difluorobenzyl)amino)benzo[b]thiophene-2-carbaldehyde (40 mg, 0.132 mmol), 4-(4-fluorophenyl)-1-(5-methyl-1,3,4-oxadiazol-2-yl)butan-2-one (36.01 mg, 0.145 mmol), 3-imino-5-methylhexanamide (20.63 mg, 0.145 mmol) in EtOH (2.0 mL) was stirred at 110° C. overnight. After the reaction was completed, the reaction concentrated in vacuum. The reaction was purified by Prep-TLC (DCM/MeOH=20/1) to give 4-(7-((3,4-difluorobenzyl)amino)benzo[b]thiophen-2-yl)-6-(4-fluorophenethyl)-2-isobutyl-5-(5-methyl-1,3,4-oxadiazol-2-yl)-1,4-dihydropyridine-3-carboxamide (40 mg, yield: 46.12%). LC-MS: m/z 658.1 (M+H)⁺.

### Step C 4-(7-((3,4-difluorobenzyl)amino)benzo[b]thiophen-2-yl)-6-(4-fluorophenethyl)-2-isobutyl-5-(5-methyl-1,3,4-oxadiazol-2-yl)nicotinamide (Compound 136)

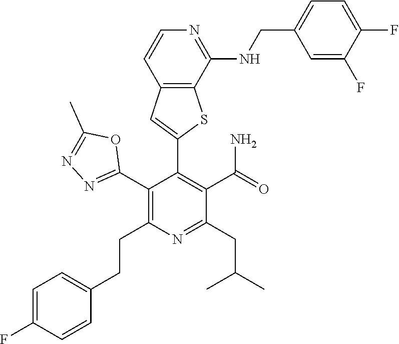 [SMILES: Cc1nnc(-c2c(CCc3ccc(F)cc3)nc(CC(C)C)c(C(N)=O)c2-c2cc3ccnc(NCc4ccc(F)c(F)c4)c3s2)o1]

To a mixture of 4-(7-((3,4-difluorobenzyl)amino)benzo[b]thiophen-2-yl)-6-(4-fluorophenethyl)-2-isobutyl-5-(5-methyl-1,3,4-oxadiazol-2-yl)-1,4-dihydropyridine-3-carboxamide (25 mg, 0.038 mmol) in dioxane (5 mL) was added MnO₂ (33.04 mg, 0.380 mmol) at room temperature, the mixture was stirred at 100° C. overnight. The reaction was filtered and concentrated in vacuum. The reaction was purified by Prep-HPLC to give 4-(7-((3,4-difluorobenzyl)amino)benzo[b]thiophen-2-yl)-6-(4-fluorophenethyl)-2-isobutyl-5-(5-methyl-1,3,4-oxadiazol-2-yl)nicotinamide (5.89 mg, yield: 23.6%).

¹H NMR (400 MHz, DMSO-d₆) δ 7.91 (d, J=1.2 Hz, 1H), 7.57 (d, J=1.2 Hz, 1H), 7.31-7.40 (m, 2H), 7.28 (s, 1H), 7.17-7.22 (m, 1H), 7.03-7.16 (m, 6H), 6.31-6.39 (m, 2H), 4.39-4.44 (m, 2H), 2.95-3.05 (m, 4H), 2.76 (d, J=7.2 Hz, 2H), 2.30-2.38 (m, 4H), 0.95 (d, J=6.8 Hz, 6H). ¹⁹F NMR (377 MHz, DMSO-d₆) δ −141.74, −139.01, −117.26. LC-MS: m/z 656.1 (M+H)⁺.

### Example 13

### (R)-4-(7-((2,3-dihydro-1H-inden-1-yl)amino)benzo[b]thiophen-2-yl)-6-(4-fluorophenethyl)-2-isobutyl-5-(5-methyl-1,3,4-oxadiazol-2-yl)nicotinamide (Compound 137)

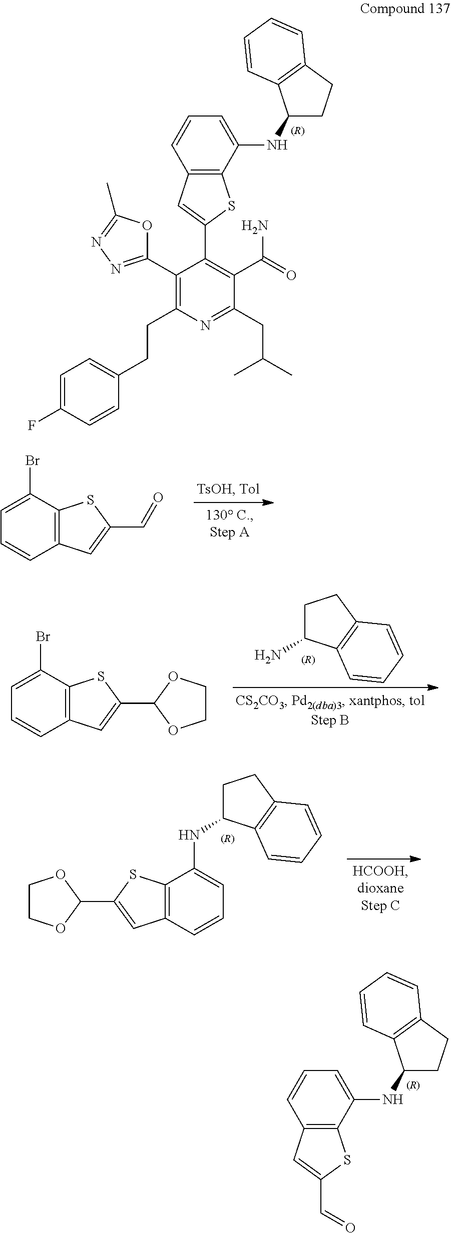

### Step A 2-(7-bromobenzo[b]thiophen-2-yl)-1,3-dioxolane

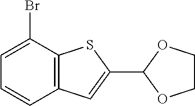 [SMILES: Brc1cccc2cc(C3OCCO3)sc12]

To a mixture of 7-bromobenzo[b]thiophene-2-carbaldehyde (650 mg, 2.696 mmol), ethylene glycol (0.451 mL, 8.088 mmol) in toluene (25 mL) was added TsOH (51.28 mg, 0.270 mmol) at rt. The mixture was stirred at 130° C. overnight. The reaction mixture was cooled down to room temperature, filtered and concentrated in vacuum. The reaction mixture was purified by column chromatography (PE/EA=1/10) to give 2-(7-bromobenzo[b]thiophen-2-yl)-1,3-dioxolane (360 mg, 46.83%). LC-MS: m/z 285.0 (M+H)⁺.

### Step B (R)—N-(2,3-dihydro-1H-inden-1-yl)-2-(1,3-dioxolan-2-yl)benzo[b]thiophen-7-amine

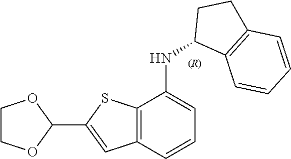

To a mixture of 2-(7-bromobenzo[b]thiophen-2-yl)-1,3-dioxolane (110 mg, 0.386 mmol), (R)-2,3-dihydro-1H-inden-1-amine (78.53 mg, 0.463 mmol) and Cs₂CO₃ (377.28 mg, 1.157 mmol) in toluene (3.0 mL) was added Xantphos (22.30 mg, 0.039 mmol) and Pd₂(dba)₃ (35.30 mg, 0.039 mmol) at room temperature. The mixture was stirred at 130° C. overnight. The reaction mixture was cooled down to room temperature, quenched with H₂O (20 mL) and extracted with ethyl acetate (20 mL×3). The organic layer was combined and washed with brine (20 mL×2), dried over sodium sulfate, filtered and concentrated in vacuum. The reaction mixture was purified by column chromatography (PE/EA=10/1) to give (R)—N-(2,3-dihydro-1H-inden-1-yl)-2-(1,3-dioxolan-2-yl)benzo[b]thiophen-7-amine (90 mg, 69.14%). LC-MS: m/z 338.1 (M+H)⁺.

### Step C (R)-7-((2,3-dihydro-1H-inden-1-yl)amino)benzo[b]thiophene-2-carbaldehyde

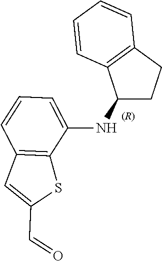

A mixture of (R)—N-(2,3-dihydro-1H-inden-1-yl)-2-(1,3-dioxolan-2-yl)benzo[b]thiophen-7-amine (90 mg, 0.267 mmol), HCOOH (2 mL) in dioxane (1 mL) was stirred at room temperature for 2 hrs. The reaction mixture was quenched with H₂O (25 mL) and extracted with ethyl acetate (20 mL×3). The organic layer was combined and washed with brine (20 mL×2), dried over sodium sulfate, filtered and concentrated in vacuum. The reaction was purified by column chromatography (PE/EA=20/1) to give (R)-7-((2,3-dihydro-1H-inden-1-yl)amino)benzo[b]thiophene-2-carbaldehyde (30 mg, 38.34%). LC-MS: m/z 294.2 (M+H)⁺.

### (R)-4-(7-((2,3-dihydro-1H-inden-1-yl)amino)benzo[b]thiophen-2-yl)-6-(4-fluorophenethyl)-2-isobutyl-5-(5-methyl-1,3,4-oxadiazol-2-yl)nicotinamide (Compound 137)

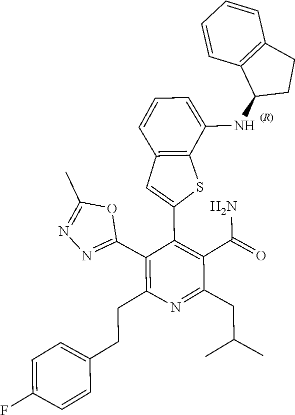

Compound 137 was then synthesized using a similar procedure described in the Example 12 above by using (R)-7-((2,3-dihydro-1H-inden-1-yl)amino)benzo[b]thiophene-2-carbaldehyde and the appropriate materials.

¹H NMR (400 MHz, DMSO-d₆) δ 57.89 (s, 1H), 7.56 (s, 1H), 7.01-7.30 (m, 12H), 6.78 (d, J=7.6 Hz, 1H), 5.77 (d, J=8.8 Hz, 1H), 5.18 (dd, J=8.0 Hz, J=15.6 Hz, 1H), 3.30 (s, 2H), 2.94-3.00 (m, 4H), 2.80-2.91 (m, 1H), 2.75 (d, J=7.2 Hz, 2H), 2.31 (s, 3H), 1.92-2.06 (m, 1H), 0.94 (d, J=6.8 Hz, 6H). ¹⁹F NMR (377 MHz, DMSO-d₆) δ −117.28. LC-MS: m/z 646.1 (M+H)⁺.

### Example 14

### 4-(7-cyano-1-benzothiophen-2-yl)-6-[2-(p-fluorophenyl)ethyl]-2-isobutyl-5-(5-methyl-1,3,4-oxadiazol-2-yl)nicotinamide (Compound 138)

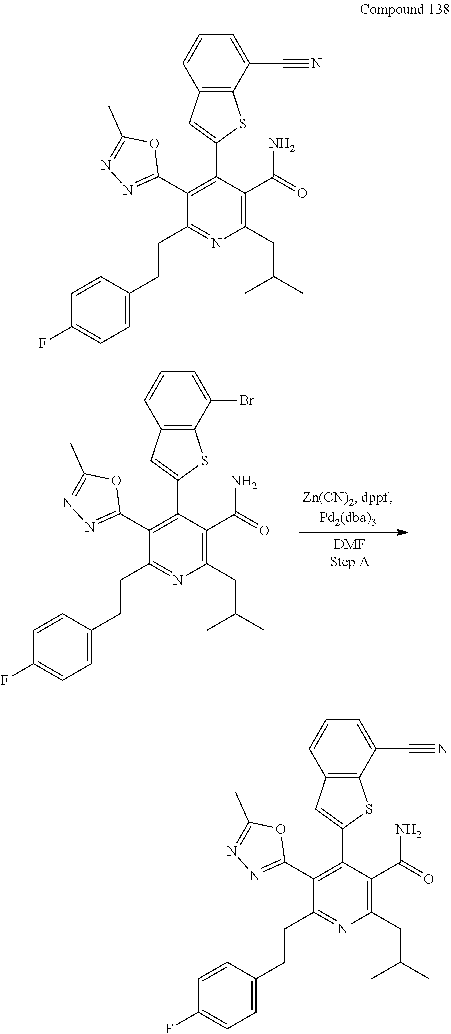

### Step A 4-(7-cyano-1-benzothiophen-2-yl)-6-[2-(p-fluorophenyl)ethyl]-2-isobutyl-5-(5-methyl-1,3,4-oxadiazol-2-yl)nicotinamide (Compound 138)

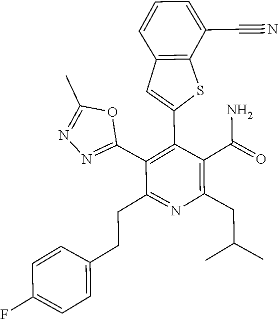 [SMILES: Cc1nnc(-c2c(CCc3ccc(F)cc3)nc(CC(C)C)c(C(N)=O)c2-c2cc3cccc(C#N)c3s2)o1]

To a mixture of 4-(7-bromobenzo[b]thiophen-2-yl)-6-(4-fluorophenethyl)-2-isobutyl-5-(5-methyl-1,3,4-oxadiazol-2-yl)nicotinamide (60 mg, 0.101 mmol), Zn(CN)₂ (118.7 mg, 1.011 mmol) in DMF (4 mL) were added bis(cyclopentyldiphenylphosphane) iron(0) (5.7 mg, 0.010 mmol) and Pd₂(dba)₃ (9.3 mg, 0.010 mmol) at room temperature, the mixture was stirred at 125° C. overnight. A The reaction mixture was cooled down to room temperature, quenched with H₂O (25 mL) and extracted with EtOAc (20 mL×3). The organic layer was combined and washed with brine (20 mL×2), dried over Na₂SO₄, filtered and concentrated in vacuum. The residue was purified by Prep-HPLC to give 4-(7-cyano-1-benzothiophen-2-yl)-6-[2-(p-fluorophenyl)ethyl]-2-isobutyl-5-(5-methyl-1,3,4-oxadiazol-2-yl)nicotinamide (31.30 mg, 57.38%).

¹H NMR (400 MHz, DMSO-d₆) δ 8.25 (d, J=8.0 Hz, 1H), 7.93-8.02 (m, 2H), 7.56-7.67 (m, 2H), 7.50 (s, 1H), 7.11-7.16 (m, 2H), 7.06 (t, J=8.8 Hz, 2H), 2.94-3.09 (m, 4H), 2.76 (d, J=7.2 Hz, 2H), 2.25-2.38 (m, 4H), 0.95 (d, J=6.4 Hz, 6H). ¹⁹F NMR (377 MHz, DMSO-d₆) δ −117.24. LC-MS: m/z 540.2 (M+H)⁺.

### Example 15

### 4-(4-aminobenzo[b]thiophen-2-yl)-6-(4-fluorophenethyl)-2-isobutyl-5-(5-methyl-1,3,4-oxadiazol-2-yl)nicotinamide (Compound 139)

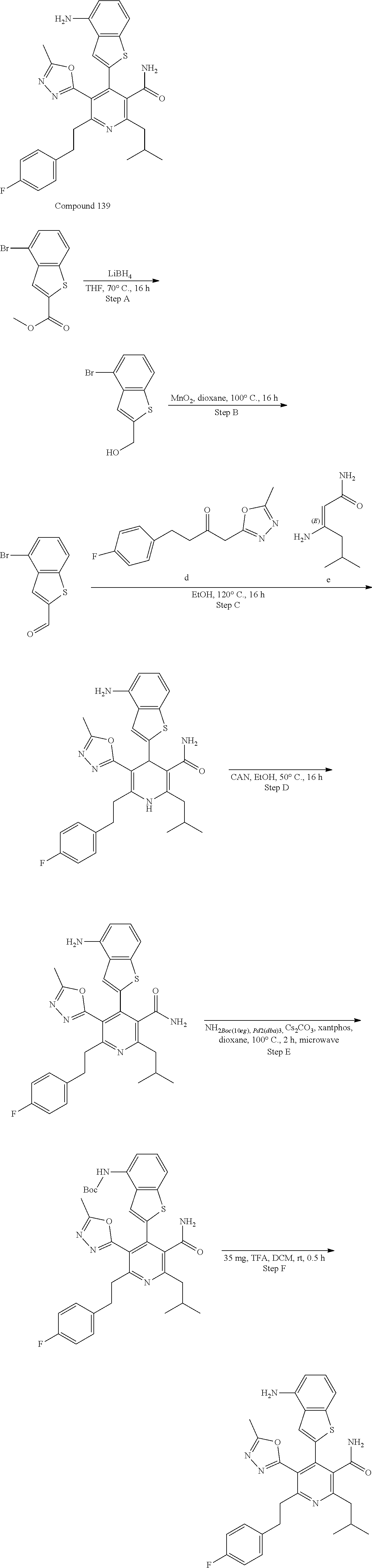

### Step A (4-bromobenzo[b]thiophen-2-yl)methanol

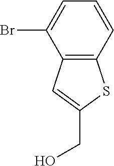 [SMILES: OCc1cc2c(Br)cccc2s1]

To a mixture of methyl 4-bromobenzo[b]thiophene-2-carboxylate (800 mg, 36.195 mmol) in THF (10 mL) was added LiBH₄ (2.17 g, 54.292 mmol) at 70° C. The mixture was stirred at 70° C. for 16 h. After the reaction was completed, the reaction was quenched with H₂O (20 mL) and extracted with ethyl acetate (10 mL×3). The organic layer was combined and washed with brine (30 mL×2), dried over sodium sulfate, filtered and concentrated in vacuum. The reaction was purified by column chromatography (PE/EA=10/1) to give (4-bromobenzo[b]thiophen-2-yl)methanol (320 mg, 44.61%). ¹H NMR (400 MHz, DMSO-d₆) δ 7.96 (d, J=8.0 Hz, 1H), 7.57-7.59 (m, 1H), 7.22-7.30 (m, 2H), 5.75 (br s, 1H), 4.78 (s, 2H).

### Step B 4-bromobenzo[b]thiophene-2-carbaldehyde

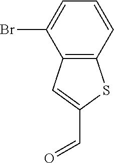 [SMILES: O=Cc1cc2c(Br)cccc2s1]

To a mixture of (4-bromobenzo[b]thiophen-2-yl)methanol (200 mg, 0.82 mmol) and MnO₂ (286.08 mg, 3.291 mmol) in dioxane (2 mL). The reaction mixture stirred at 100° C. for 16 h. After the reaction was completed, the reaction was filtered and concentrated in vacuum. The reaction was purified by column chromatography (PE/EA=10/1) to give 4-bromobenzo[b]thiophene-2-carbaldehyde (130 mg, 65.54%). ¹H NMR (400 MHz, DMSO-d₆) δ 10.20 (s, 1H), 8.44 (s, 1H), 8.16 (d, J=8.0 Hz, 1H), 7.77 (d, J=8.0 Hz, 1H), 7.51 (t, J=8.0 Hz, 1H).

### Step C 4-(4-bromobenzo[b]thiophen-2-yl)-6-(4-fluorophenethyl)-2-isobutyl-5-(5-methyl-1,3,4-oxadiazol-2-yl)-1,4-dihydropyridine-3-carboxamide

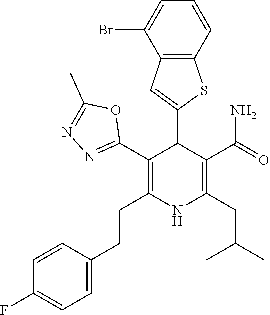 [SMILES: Cc1nnc(C2=C(CCc3ccc(F)cc3)NC(CC(C)C)=C(C(N)=O)C2c2cc3c(Br)cccc3s2)o1]

A mixture of (4-bromobenzo[b]thiophen-2-yl)methanol (130 mg, 0.539 mmol), 4-(4-fluorophenyl)-1-(5-methyl-1,3,4-oxadiazol-2-yl)butan-2-one (134 mg, 0.539 mmol) and (E)-3-amino-5-methylhex-2-enamide (77 mg, 0.539 mmol) in EtOH (2 mL) was stirred at 110° C. for 16 h. After the reaction was completed, the reaction was quenched with H₂O (10 mL) and extracted with ethyl acetate (5 mL×3). The organic layer was combined and washed with brine (15 mL×2), dried over sodium sulfate, filtered and concentrated in vacuum. The reaction was purified by Prep-TLC (PE/EA=3/1) to give 4-(4-bromobenzo[b]thiophen-2-yl)-6-(4-fluorophenethyl)-2-isobutyl-5-(5-methyl-1,3,4-oxadiazol-2-yl)-1,4-dihydropyridine-3-carboxamide (120 mg, 37.37%). LC-MS: m/z 595.0 (M+H)⁺.

### Step D 4-(4-bromobenzo[b]thiophen-2-yl)-6-(4-fluorophenethyl)-2-isobutyl-5-(5-methyl-1,3,4-oxadiazol-2-yl)nicotinamide

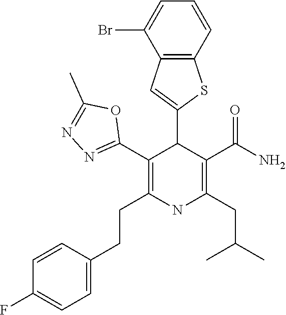 [SMILES: Cc1nnc(C2=C(CCc3ccc(F)cc3)NC(CC(C)C)=C(C(N)=O)C2c2cc3c(Br)cccc3s2)o1]

A mixture of 4-(4-bromobenzo[b]thiophen-2-yl)-6-(4-fluorophenethyl)-2-isobutyl-5-(5-methyl-1,3,4-oxadiazol-2-yl)-1,4-dihydropyridine-3-carboxamide (120 mg, 0.498 mmol), CAN (1 g, 1.991 mmol) in EtOH (2 mL) was stirred at 50° C. for 16 h. After the reaction was completed, the reaction was quenched with H₂O (10 mL) and extracted with ethyl acetate (5 mL×3). The organic layer was combined and washed with brine (15 mL×2), dried over sodium sulfate, filtered and concentrated in vacuum. The reaction was purified by Prep-TLC (PE/EA=3/1) to give 4-(4-bromobenzo[b]thiophen-2-yl)-6-(4-fluorophenethyl)-2-isobutyl-5-(5-methyl-1,3,4-oxadiazol-2-yl)nicotinamide (80 mg, 26.99%). LC-MS: m/z 593.0 (M+H)⁺.

### Step E tert-butyl (2-(3-carbamoyl-6-(4-fluorophenethyl)-2-isobutyl-5-(5-methyl-1,3,4-oxadiazol-2-yl)pyridin-4-yl)benzo[b]thiophen-4-yl)carbamate

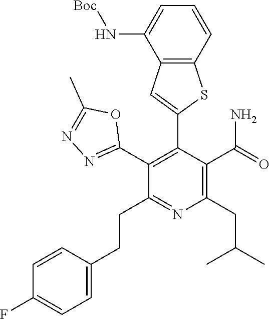 [SMILES: Cc1nnc(-c2c(CCc3ccc(F)cc3)nc(CC(C)C)c(C(N)=O)c2-c2cc3c(NC(=O)OC(C)(C)C)cccc3s2)o1]

A mixture of 4-(4-bromobenzo[b]thiophen-2-yl)-6-(4-fluorophenethyl)-2-isobutyl-5-(5-methyl-1,3,4-oxadiazol-2-yl)nicotinamide (65 mg, 0.110 mmol) and Pd₂(dba)₃ (10.03 mg, 0.011 mmol), Cs₂CO₃ (107.05 mg, 0.329 mmol), Xantphos (12.67 mg, 0.022 mmol), 2-methylpropan-2-yl aminomethanoate (128.30 mg, 1.095 mmol) in dioxane (5 mL) was stirred at 100° C. for 2 h in microwave. After the reaction was completed, the reaction was quenched with H₂O (10 mL) and extracted with ethyl acetate (5 mL×3). The organic layer was combined and washed with brine (15 mL×2), dried over sodium sulfate, filtered and concentrated in vacuum. The reaction was purified by Prep-TLC (PE/EA=3/1) to give tert-butyl (2-(3-carbamoyl-6-(4-fluorophenethyl)-2-isobutyl-5-(5-methyl-1,3,4-oxadiazol-2-yl)pyridin-4-yl)benzo[b]thiophen-4-yl)carbamate (35 mg, 50.75%). LC-MS: m/z 630.0 (M+H)⁺.

### Step F 4-(4-aminobenzo[b]thiophen-2-yl)-6-(4-fluorophenethyl)-2-isobutyl-5-(5-methyl-1,3,4-oxadiazol-2-yl)nicotinamide (Compound 139)

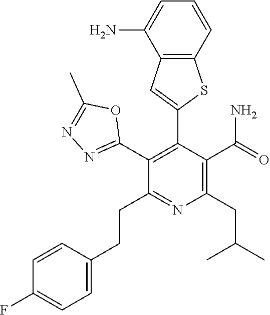 [SMILES: Cc1nnc(-c2c(CCc3ccc(F)cc3)nc(CC(C)C)c(C(N)=O)c2-c2cc3c(N)cccc3s2)o1]

A mixture of tert-butyl (2-(3-carbamoyl-6-(4-fluorophenethyl)-2-isobutyl-5-(5-methyl-1,3,4-oxadiazol-2-yl)pyridin-4-yl)benzo[b]thiophen-4-yl)carbamate (35 mg, 0.056 mmol) in DCM (3 mL) and TFA (0.5 mL) was stirred at room temperature for 0.5 h. After the reaction was completed, the reaction was filtered and concentrated in vacuum. The reaction was purified by Prep-HPLC (TFA) to give 4-(4-aminobenzo[b]thiophen-2-yl)-6-(4-fluorophenethyl)-2-isobutyl-5-(5-methyl-1,3,4-oxadiazol-2-yl)nicotinamide (2.29 mg, 7.64%).

¹H NMR (400 MHz, DMSO-d₆) δ 7.83 (br s, 1H), 7.51 (br s, 1H), 7.43 (s, 1H), 6.98-7.15 (m, 6H), 6.48-6.53 (m, 1H), 2.97 (s, 4H), 2.75 (d, J=7.2 Hz, 2H), 2.27-2.36 (m, 4H), 0.94 (d, J=6.4 Hz, 6H). ¹⁹F NMR (377 MHz, DMSO-d₆) δ −117.28. LC-MS: m/z 530.2 (M+H)⁺.

### Example 16

### 4-(5-{[(R)-1-indanylamino]methyl}-2-thienyl)-6-[2-(p-fluorophenyl)ethyl]-2-isobutyl-5-(5-methyl-1,3,4-oxadiazol-2-yl)nicotinamide (Compound 140)

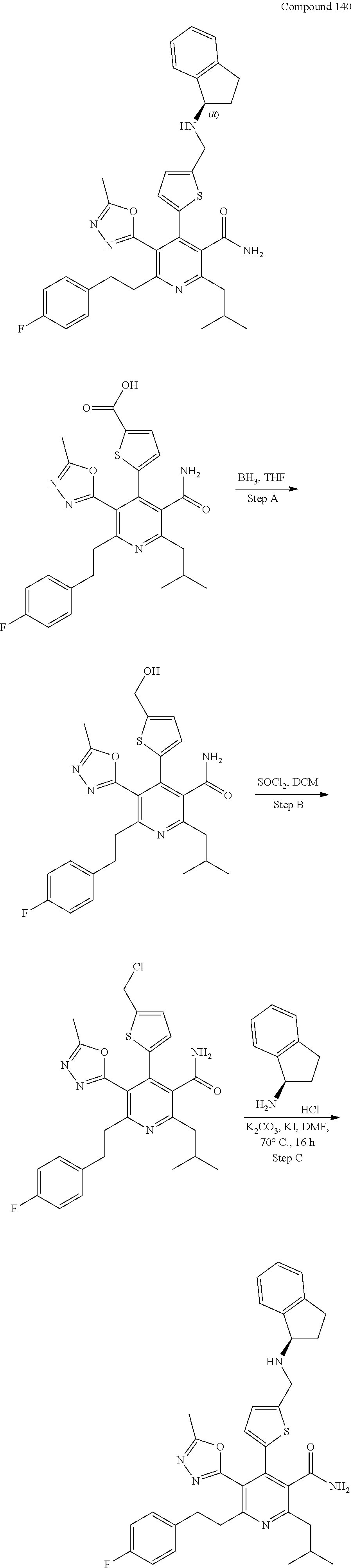

### Step A 6-[2-(4-fluorophenyl)ethyl]-4-[5-(hydroxymethyl)thiophen-2-yl]-5-(5-methyl-1,3,4-oxadiazol-2-yl)-2-(2-methylpropyl)pyridine-3-carboxamide

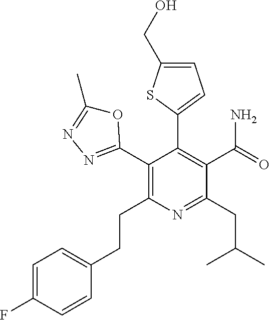 [SMILES: Cc1nnc(-c2c(CCc3ccc(F)cc3)nc(CC(C)C)c(C(N)=O)c2-c2ccc(CO)s2)o1]

To a solution of 5-{5-carbamoyl-2-[2-(4-fluorophenyl)ethyl]-3-(5-methyl-1,3,4-oxadiazol-2-yl)-6-(2-methylpropyl)pyridin-4-yl}thiophene-2-carboxylic acid (390 mg, 0.767 mmol) in THF (3 mL) was added 1 M BH₃ in THF (7.669 mL), and the reaction mixture was stirred at room temperature for 2 hrs. The mixture was quenched with MeOH (10 mL) and diluted with EtOAc (50 mL) and water (20 mL). The organic layer was separated, washed with water (20 mL×2), the organic layer was separated, dried over Na₂SO₄ and then filtered. The organic layer was collected and concentrated in vacuo. The residue was purified by silica gel column chromatography eluting with methanol in dichloroform (DCM:MeOH=30:1). The organic layer was collected, concentrated in vacuo to give 6-[2-(4-fluorophenyl)ethyl]-4-[5-(hydroxymethyl)thiophen-2-yl]-5-(5-methyl-1,3,4-oxadiazol-2-yl)-2-(2-methylpropyl)pyridine-3-carboxamide (330 mg, 87.01%). LC-MS: m/z 495.2 (M+H)⁺.

### Step B 4-[5-(chloromethyl)thiophen-2-yl]-6-[2-(4-fluorophenyl)ethyl]-5-(5-methyl-1,3,4-oxadiazol-2-yl)-2-(2-methylpropyl)pyridine-3-carboxamide

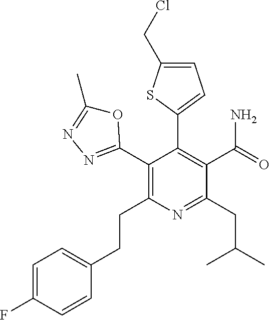 [SMILES: Cc1nnc(-c2c(CCc3ccc(F)cc3)nc(CC(C)C)c(C(N)=O)c2-c2ccc(CCl)s2)o1]

To a solution of 6-[2-(4-fluorophenyl)ethyl]-4-[5-(hydroxymethyl)thiophen-2-yl]-5-(5-methyl-1,3,4-oxadiazol-2-yl)-2-(2-methylpropyl)pyridine-3-carboxamide (330 mg, 0.667 mmol) in DCM (5 mL) was added SOCl₂ (158.74 mg, 1.334 mmol) at 0° C. and the reaction mixture was stirred at room temperature for 2 hrs. The mixture was diluted with EtOAc (50 mL) and water (30 mL). The organic layer was separated, washed with water (30 mL×2), the organic layer was separated, dried over Na₂SO₄ and then filtered. The organic layer was collected and concentrated in vacuo. The residue was purified by silica gel column chromatography eluting with methanol in dichloroform (DCM:MeOH=30:1). The organic layer was collected, concentrated in vacuo to give 4-[5-(chloromethyl)thiophen-2-yl]-6-[2-(4-fluorophenyl)ethyl]-5-(5-methyl-1,3,4-oxadiazol-2-yl)-2-(2-methylpropyl)pyridine-3-carboxamide (160 mg, 46.74%). LC-MS: m/z 513.2 (M+H)⁺.

### Step C 4-(5-{[(R)-1-indanylamino]methyl}-2-thienyl)-6-[2-(p-fluorophenyl)ethyl]-2-isobutyl-5-(5-methyl-1,3,4-oxadiazol-2-yl)nicotinamide (Compound 140)

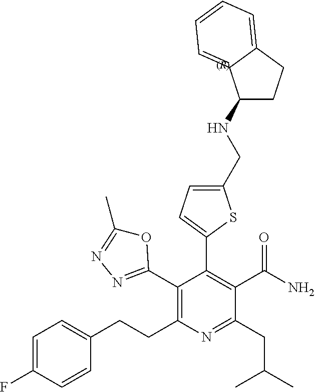

To a solution of 4-[5-(chloromethyl)thiophen-2-yl]-6-[2-(4-fluorophenyl)ethyl]-5-(5-methyl-1,3,4-oxadiazol-2-yl)-2-(2-methylpropyl)pyridine-3-carboxamide (30 mg, 0.058 mmol) in DMF (1 mL) were added (1R)-2,3-dihydro-1H-inden-1-amine HCl (7.79 mg, 0.058 mmol), K₂CO₃ (24.24 mg, 0.175 mmol) and KI (0.97 mg, 0.006 mmol), and the reaction mixture was stirred at 70° C. for 18 hrs. The reaction mixture was cooled down to room temperature, filtered and purified by Prep-HPLC (FA) to give 4-(5-{[(R)-1-indanylamino]methyl}-2-thienyl)-6-[2-(p-fluorophenyl)ethyl]-2-isobutyl-5-(5-methyl-1,3,4-oxadiazol-2-yl)nicotinamide. ¹H NMR (400 MHz, DMSO-d₆) δ 7.93 (s, 1H), 7.52-7.59 (m, 1H), 7.32-7.38 (m, 1H), 7.18-7.29 (m, 3H), 7.00-7.13 (m, 4H), 6.90-6.98 (m, 2H), 4.11-4.18 (m, 1H), 3.99 (s, 2H), 2.89-3.01 (m, 5H), 2.68-2.80 (m, 3H), 2.22-2.34 (m, 5H), 1.75-1.86 (m, 1H), 0.93 (d, J=6.4 Hz, 6H). ¹⁹F NMR (377 MHz, DMSO-d₆) δ −117.23. LC-MS: m/z 610.2 (M+H)⁺.

### Example 17

### 4-{5-[(3,4-difluorobenzylamino)methyl]-2-thienyl}-6-[2-(p-fluorophenyl)ethyl]-2-isobutyl-5-(5-methyl-1,3,4-oxadiazol-2-yl)nicotinamide (Compound 141)

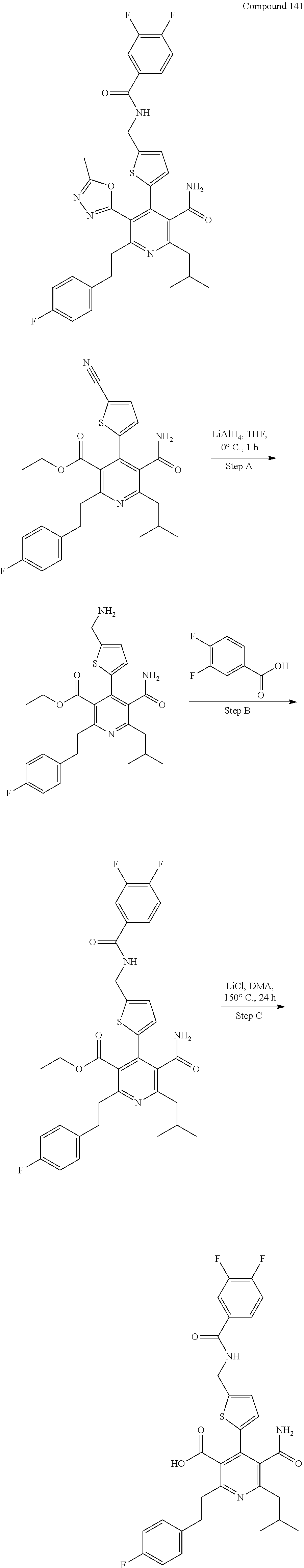 [SMILES: Cc1nnc(-c2c(CCc3ccc(F)cc3)nc(CC(C)C)c(C(N)=O)c2-c2ccc(CNC(=O)c3ccc(F)c(F)c3)s2)o1 ; CCOC(=O)c1c(CCc2ccc(F)cc2)nc(CC(C)C)c(C(N)=O)c1-c1ccc(C#N)s1 ; CCOC(=O)c1c(CCc2ccc(F)cc2)nc(CC(C)C)c(C(N)=O)c1-c1ccc(CN)s1 ; O=C(O)c1ccc(F)c(F)c1 ; CCOC(=O)c1c(CCc2ccc(F)cc2)nc(CC(C)C)c(C(N)=O)c1-c1ccc(CNC(=O)c2ccc(F)c(F)c2)s1 ; CC(C)Cc1nc(CCc2ccc(F)cc2)c(C(=O)O)c(-c2ccc(CNC(=O)c3ccc(F)c(F)c3)s2)c1C(N)=O]

### Step A ethyl 4-(5-(aminomethyl)thiophen-2-yl)-5-carbamoyl-2-(4-fluorophenethyl)-6-isobutylnicotinate

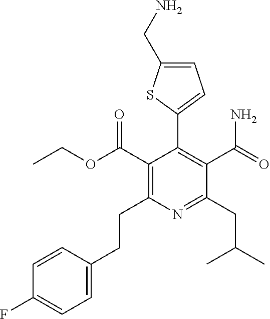 [SMILES: CCOC(=O)c1c(CCc2ccc(F)cc2)nc(CC(C)C)c(C(N)=O)c1-c1ccc(CN)s1]

To a solution of ethyl 5-carbamoyl-4-(5-cyanothiophen-2-yl)-2-(4-fluorophenethyl)-6-isobutylnicotinate (361 mg, 0.753 mmol, synthesized using a similar procedure described in the Example 1 above by using the appropriate materials) in THF (4 mL) was added LiAlH₄ (28.57 mg, 0.753 mmol), the reaction mixture was stirred at 0° C. for 20 min. The reaction mixture was quenched with H₂O (10 mL) and extracted with EtOAc (25 mL×3). The organic layer was combined and washed with brine (20 mL×2), dried over Na₂SO₄, filtered and concentrated in vacuum. The residue was purified by column chromatography on silica gel (DCM/MeOH=20/1) to give ethyl 4-(5-(aminomethyl)thiophen-2-yl)-5-carbamoyl-2-(4-fluorophenethyl)-6-isobutylnicotinate (190 mg, 52.34%). MS: m/z 484.2 (M+H)⁺.

### Step B ethyl 5-carbamoyl-4-(5-((3,4-difluorobenzamido)methyl)thiophen-2-yl)-2-(4-fluorophenethyl)-6-isobutylnicotinate

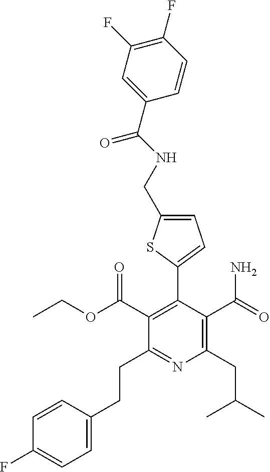 [SMILES: CCOC(=O)c1c(CCc2ccc(F)cc2)nc(CC(C)C)c(C(N)=O)c1-c1ccc(CNC(=O)c2ccc(F)c(F)c2)s1]

To a solution of ethyl 4-(5-(aminomethyl)thiophen-2-yl)-5-carbamoyl-2-(4-fluorophenethyl)-6-isobutylnicotinate (190 mg, 0.393 mmol) in DMF (2 mL) were added 3,4-difluorobenzoic acid (62.12 mg, 0.393 mmol), PyBOP (408.91 mg, 0.786 mmol) and DIEA (152.05 mg, 1.179 mmol), the reaction mixture was stirred at room temperature for 2 hrs. The mixture was diluted with water (30 ml) and extracted with EtOAc (20 mL×3). The organic layer was combined and washed with brine (30 mL×2), dried over Na₂SO₄, filtered and concentrated in vacuum. The mixture was purified by Prep-HPLC to give ethyl 5-carbamoyl-4-(5-((3,4-difluorobenzamido)methyl)thiophen-2-yl)-2-(4-fluorophenethyl)-6-isobutylnicotinate (43 mg, 17.49%). ¹H NMR (400 MHz, DMSO-d₆) δ 9.35 (t, J=6.0 Hz, 1H), 7.87-7.94 (m, 1H), 7.74-7.82 (m, 2H), 7.47-7.61 (m, 2H), 7.18-7.23 (m, 2H), 7.05-7.10 (m, 2H), 6.99 (d, J=3.6 Hz, 1H), 6.93 (d, J=3.6 Hz, 1H), 4.61 (d, J=6.0 Hz, 2H), 3.99 (q, J=7.2 Hz, 2H), 2.93-2.99 (m, 4H), 2.63 (d, J=6.8 Hz, 2H), 2.18-2.28 (m, 1H), 0.86-0.93 (m, 9H). ¹⁹F NMR (377 MHz, DMSO-d₆) δ −117.37, −134.32, −137.80. MS: m/z 624.3 (M+H)⁺.

### Step C 5-carbamoyl-4-(5-((3,4-difluorobenzamido)methyl)thiophen-2-yl)-2-(4-fluorophenethyl)-6-isobutylnicotinic acid

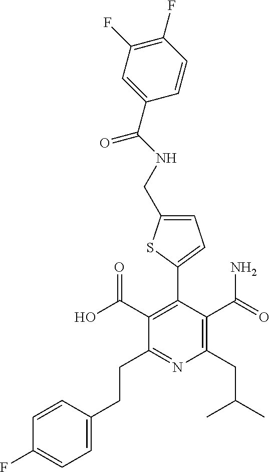 [SMILES: CC(C)Cc1nc(CCc2ccc(F)cc2)c(C(=O)O)c(-c2ccc(CNC(=O)c3ccc(F)c(F)c3)s2)c1C(N)=O]

To a solution of ethyl 5-carbamoyl-4-(5-((3,4-difluorobenzamido)methyl)thiophen-2-yl)-2-(4-fluorophenethyl)-6-isobutylnicotinate (43 mg, 0.07 mmol) in DMA (1 mL) was added LiCl (11.70 mg, 0.27 mmol), the reaction mixture was stirred at stirred at 150° C. for 24 hrs. The reaction mixture was cooled down to room temperature, filtered and the filtrate was purified by Prep-HPLC (0.1% FA/H₂O/CH₃CN) to give 5-carbamoyl-4-(5-((3,4-difluorobenzamido)methyl)thiophen-2-yl)-2-(4-fluorophenethyl)-6-isobutylnicotinic acid (28 mg, 68.29%). LC-MS: m/z 596.1 (M+H)⁺.

### 4-{5-[(3,4-difluorobenzylamino)methyl]-2-thienyl}-6-[2-(p-fluorophenyl)ethyl]-2-isobutyl-5-(5-methyl-1,3,4-oxadiazol-2-yl)nicotinamide (Compound 141)

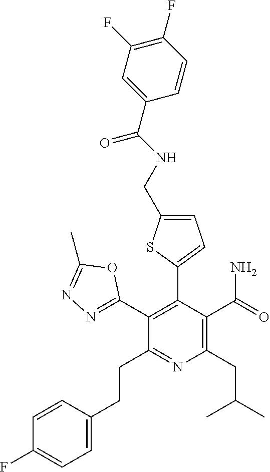 [SMILES: Cc1nnc(-c2c(CCc3ccc(F)cc3)nc(CC(C)C)c(C(N)=O)c2-c2ccc(CNC(=O)c3ccc(F)c(F)c3)s2)o1]

Compound 141 was then synthesized using a similar procedure described in the Example 4 above by using 5-carbamoyl-4-(5-((3,4-difluorobenzamido)methyl)thiophen-2-yl)-2-(4-fluorophenethyl)-6-isobutylnicotinic acid and appropriate materials.

¹H NMR (400 MHz, DMSO-d₆) δ 9.26 (t, J=7.6 Hz, 1H), 7.82-7.93 (m, 2H), 7.71-7.77 (m, 1H), 7.54-7.64 (m, 2H), 6.98-7.12 (m, 4H), 6.87-6.93 (m, 2H), 4.53 (d, J=5.2 Hz, 2H), 2.91-2.99 (m, 4H), 2.64-2.73 (m, 2H), 2.25-2.33 (m, 4H), 0.92 (d, J=6.4 Hz, 6H). ¹⁹F NMR (377 MHz, DMSO-d₆) δ −117.33, −134.31, −137.79. LC-MS: m/z 641.3 (M+H)⁺.

### Example 18

### 4-{1-[(3,4-difluorophenyl)methoxy]-5-indanyl}-6-[2-(p-fluorophenyl)ethyl]-2-isobutyl-5-(5-methyl-1,3,4-oxadiazol-2-yl)nicotinamide (Compound 142)

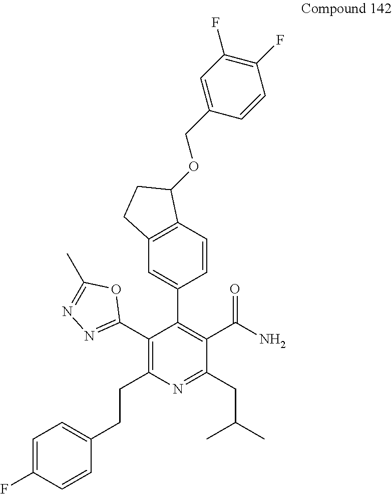 [SMILES: Cc1nnc(-c2c(CCc3ccc(F)cc3)nc(CC(C)C)c(C(N)=O)c2-c2ccc3c(c2)CCC3OCc2ccc(F)c(F)c2)o1]

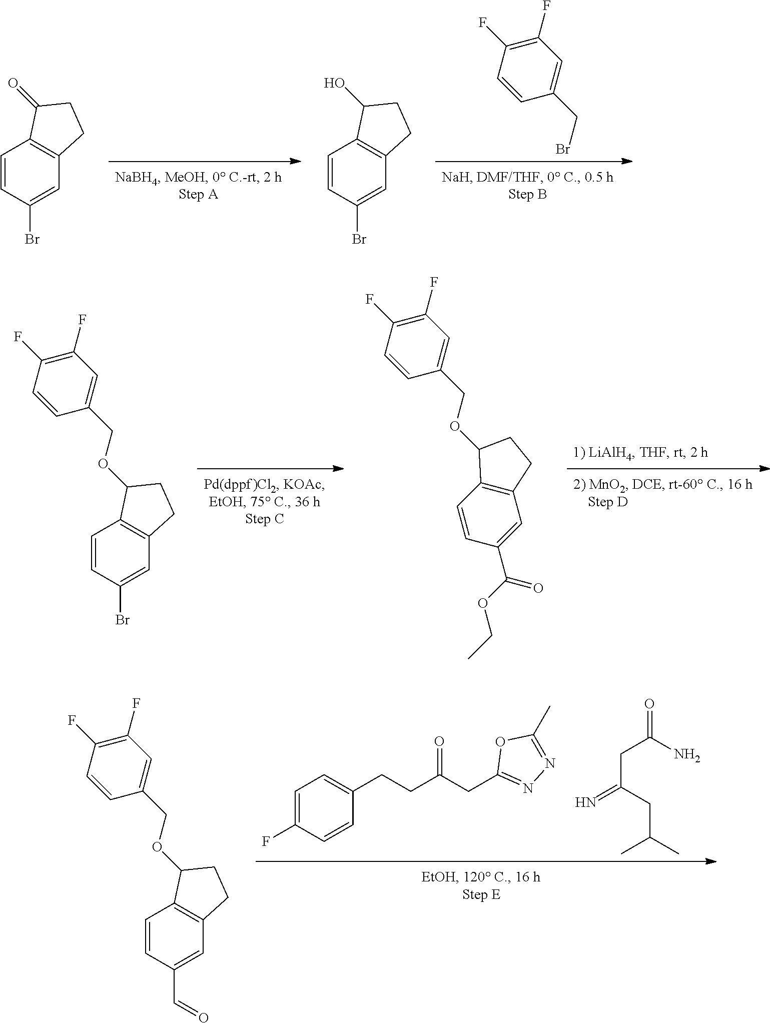

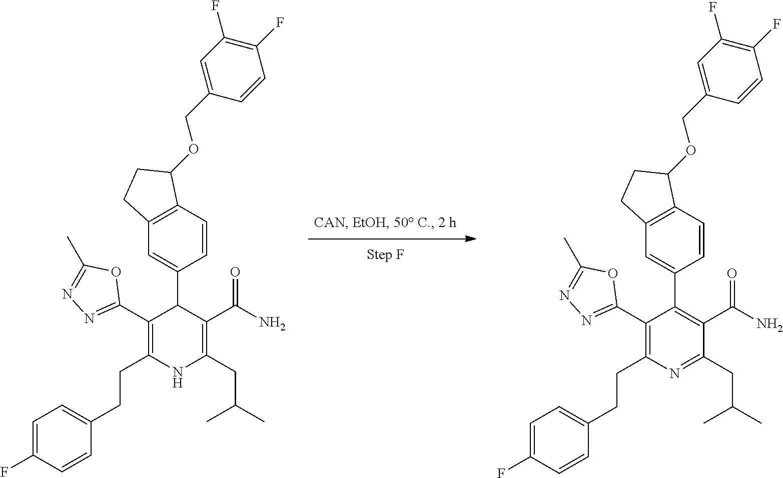 [SMILES: Cc1nnc(C2=C(CCc3ccc(F)cc3)NC(CC(C)C)=C(C(N)=O)C2c2ccc3c(c2)CCC3OCc2ccc(F)c(F)c2)o1 ; Cc1nnc(-c2c(CCc3ccc(F)cc3)nc(CC(C)C)c(C(N)=O)c2-c2ccc3c(c2)CCC3OCc2ccc(F)c(F)c2)o1]

### Step A 5-bromo-2,3-dihydro-1H-inden-1-ol

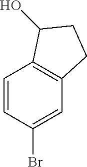 [SMILES: OC1CCc2cc(Br)ccc21]

To a solution of 5-bromo-2,3-dihydro-1H-inden-1-one (5 g, 23.69 mmol) in MeOH (500 mL) was added NaBH₄ (2.24 g, 59.22 mmol) at 0° C. The reaction mixture stirred at 0° C. for 2 hrs. The reaction mixture was quenched with Sat. NH₄Cl (200 mL) and extracted with EtOAc (200 mL*2). The organic layers were combined, dried over Na₂SO₄, filtered and concentrated under vacuum to dryness. The crude product was purified by silica gel column chromatography by using 0-10% EtOAc/hexane to give 5-bromo-2,3-dihydro-1H-inden-1-ol (4.50 g, 89.15%).

¹H NMR (400 MHz, CDCl₃) δ 7.34-7.43 (m, 2H), 7.26-7.31 (m, 1H), 5.15-5.24 (m, 1H), 2.98-3.10 (m, 1H), 2.75-2.87 (m, 1H), 2.44-2.55 (m, 1H), 1.88-2.02 (m, 1H), 1.67 (br s, 1H).

### Step B 5-bromo-1-((3,4-difluorobenzyl)oxy)-2,3-dihydro-1H-indene

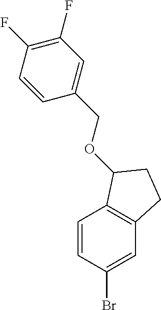 [SMILES: Fc1ccc(COC2CCc3cc(Br)ccc32)cc1F]

To a solution of 5-bromo-2,3-dihydro-1H-inden-1-ol (2.00 g, 9.39 mmol) in DMF (10 mL) and THF (10 mL), was added NaH (750 mg, 18.77 mmol, 60% dispersion in mineral oil), 4-(bromomethyl)-1,2-difluorobenzene (2.33 g, 11.26 mmol). The mixture was stirred at 0° C. for 1 hr. The mixture was diluted with EtOAc (20 mL), washed with water (40 mL). The organic layers were dried over Na₂SO₄, filtered and concentrated under vacuum to dryness. The crude product was purified by silica gel column chromatography by using 0-10% EtOAc/hexane to give 5-bromo-1-((3,4-difluorobenzyl)oxy)-2,3-dihydro-1H-indene (1.80 g, 60.0%).

¹H NMR (400 MHz, CDCl₃) δ 7.39 (s, 1H), 7.33 (t, J=8.0 Hz, 1H), 7.00-7.25 (m, 4H), 4.87-4.98 (m, 1H), 4.75-4.58 (m, 2H), 3.00-3.13 (m, 1H), 2.74-2.87 (m, 1H), 2.27-2.42 (m, 1H), 2.05-2.19 (m, 1H).

### Step C ethyl 1-((tert-butoxycarbonyl)(3,4-difluorobenzyl)amino)-2,3-dihydro-1H-indene-5-carboxylate

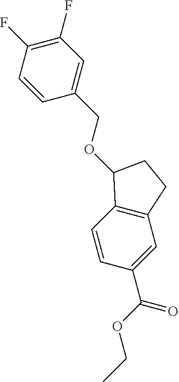 [SMILES: CCOC(=O)c1ccc2c(c1)CCC2OCc1ccc(F)c(F)c1]

A mixture of 5-bromo-1-((3,4-difluorobenzyl)oxy)-2,3-dihydro-1H-indene (1.80 g, 5.30 mmol), Pd(dppf)Cl₂ (390 mg, 0.53 mmol) and KOAc (1.54 g, 15.92 mmol) in EtOH (20 mL) was stirred at 75° C. for 16 hrs under CO. The reaction mixture was concentrated and purified by silica gel column chromatography by using 0-10% EtOAc/hexane to give ethyl 1-((3,4-difluorobenzyl)oxy)-2,3-dihydro-1H-indene-5-carboxylate (1.50 g, 89.15%).

¹H NMR (400 MHz, CDCl₃): δ 7.92-7.94 (m, 2H), 7.44 (d, J=7.6 Hz, 1H), 7.00-7.32 (m, 4H), 5.03 (t, J=5.6 Hz, 1H), 4.53-4.62 (m, 2H), 4.37 (q, J=6.8 Hz, 2H), 3.07-3.19 (m, 1H), 2.81-2.93 (m, 1H), 2.37-2.48 (m, 1H), 2.11-2.21 (m, 1H), 1.39 (t, J=6.8 Hz, 3H).

### Step D 1-((3,4-difluorobenzyl)oxy)-2,3-dihydro-1H-indene-5-carbaldehyde

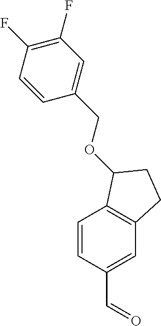 [SMILES: O=Cc1ccc2c(c1)CCC2OCc1ccc(F)c(F)c1]

To a solution of ethyl 1-((3,4-difluorobenzyl)oxy)-2,3-dihydro-1H-indene-5-carboxylate (1.5 g, crude) in THF (30 mL) were added LiAlH₄ (510 mg, 13.54 mmol), and the reaction mixture was stirred room temperature for 2 hrs. The reaction mixture was quenched with Sat. NH₄Cl (50 mL) and extracted with EtOAc (20 mL×3). The organic layers were combined, dried over Na₂SO₄, filtered and concentrated under vacuum to dryness. The reaction mixture was concentrated in vacuo to give (1-((3,4-difluorobenzyl)oxy)-2,3-dihydro-1H-inden-5-yl)methanol (1.10 g, crude). Then to the solution of (1-((3,4-difluorobenzyl)oxy)-2,3-dihydro-1H-inden-5-yl)methanol (300 mg, crude) in DCE (3 mL) were added MnO₂ (356 mg, 4.13 mmol), and the reaction mixture was stirred 60° C. for 16 hrs. The reaction mixture was cooled down to room temperature and diluted with DCM (20 mL), washed with water (40 mL). The organic residue was dried over Na₂SO₄, filtered and concentrated under vacuum to dryness. The crude product was purified by silica gel column chromatography by using 0-10% EtOAc/hexane to give 1-((3,4-difluorobenzyl)oxy)-2,3-dihydro-1H-indene-5-carbaldehyde (220 mg, 60.0%).

¹H NMR (400 MHz, CDCl₃) δ 10.00 (s, 1H), 7.70-7.82 (m, 2H), 7.55 (d, J=7.6 Hz, 1H), 7.04-7.26 (m, 3H), 5.05 (t, J=6.0 Hz, 1H), 4.52-4.67 (m, 2H), 3.07-3.22 (m, 1H), 2.83-2.95 (m, 1H), 2.39-2.52 (m, 1H), 2.09-2.23 (m, 1H).

### Step E 4-(1-((3,4-difluorobenzyl)oxy)-2,3-dihydro-1H-inden-5-yl)-6-(4-fluorophenethyl)-2-isobutyl-5-(5-methyl-1,3,4-oxadiazol-2-yl)-1,4-dihydropyridine-3-carboxamide

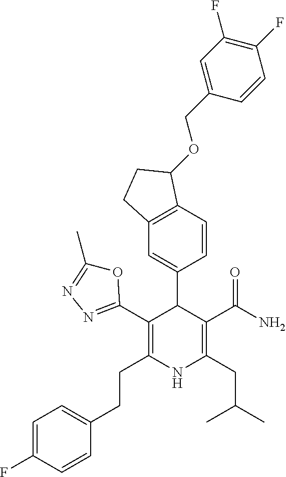 [SMILES: Cc1nnc(C2=C(CCc3ccc(F)cc3)NC(CC(C)C)=C(C(N)=O)C2c2ccc3c(c2)CCC3OCc2ccc(F)c(F)c2)o1]

A mixture of 1-((3,4-difluorobenzyl)oxy)-2,3-dihydro-1H-indene-5-carbaldehyde (70 mg, 0.24 mmol), 4-(4-fluorophenyl)-1-(5-methyl-1,3,4-oxadiazol-2-yl)butan-2-one (48 mg, 0.19 mmol) and 3-imino-5-methylhexanamide(27 mg, 0.19 mmol) in EtOH (2 mL) was stirred 120° C. for 16 hrs. The reaction mixture was cooled down to room temperature, concentrated in vacuo to give 4-(1-((3,4-difluorobenzyl)oxy)-2,3-dihydro-1H-inden-5-yl)-6-(4-fluorophenethyl)-2-isobutyl-5-(5-methyl-1,3,4-oxadiazol-2-yl)-1,4-dihydropyridine-3-carboxamide (200 mg, crude). LC-MS: m/z 643.3 (M+H)⁺.

### Step F 4-{1-[(3,4-difluorophenyl)methoxy]-5-indanyl}-6-[2-(p-fluorophenyl)ethyl]-2-isobutyl-5-(5-methyl-1,3,4-oxadiazol-2-yl)nicotinamide (Compound 142)

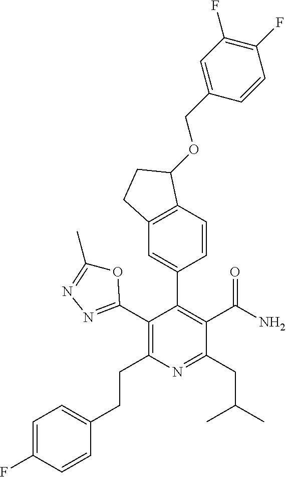 [SMILES: Cc1nnc(-c2c(CCc3ccc(F)cc3)nc(CC(C)C)c(C(N)=O)c2-c2ccc3c(c2)CCC3OCc2ccc(F)c(F)c2)o1]

To a solution of 4-(1-((3,4-difluorobenzyl)oxy)-2,3-dihydro-1H-inden-5-yl)-6-(4-fluorophenethyl)-2-isobutyl-5-(5-methyl-1,3,4-oxadiazol-2-yl)-1,4-dihydropyridine-3-carboxamide (130 mg, crude) in EtOH (2 mL) was added diammonium cerium(IV) nitrate. The reaction mixture was stirred at 50° C. for 2 hrs. A The reaction mixture was cooled down to room temperature, diluted with EtOAc (50 mL), extracted with water (30 mL). The organic residue was dried over Na₂SO₄, filtered and concentrated under vacuum to dryness, filtered and concentrated under vacuum to dryness. The crude product was purified by Prep-HPLC (NH₃H₂O) to give 4-{1-[(3,4-difluorophenyl)methoxy]-5-indanyl}-6-[2-(p-fluorophenyl)ethyl]-2-isobutyl-5-(5-methyl-1,3,4-oxadiazol-2-yl)nicotinamide (6.98 mg, 3.0%).

¹H NMR (400 MHz, DMSO-d₆) δ 7.73 (br s, 1H), 7.17-7.47 (m, 5H), 6.89-6.12 (m, 6H), 4.95-4.98 (m, 1H), 4.47-4.61 (m, 2H), 2.81-3.00 (m, 5H), 2.65-2.77 (m, 3H), 2.17-2.38 (m, 5H), 1.92-2.05 (m, 1H), 0.94 (d, J=6.8 Hz, 6H). ¹⁹F NMR (377 MHz, DMSO-d₆) δ −117.34, −138.93, −140.72. LC-MS: m/z 641.3 (M+H)⁺.

### Example 19

### 4-(1-{[(3,4-difluorophenyl)methyl]amino}-5-indanyl)-6-[2-(p-fluorophenyl)ethyl]-2-isobutyl-5-(5-methyl-1,3,4-oxadiazol-2-yl)nicotinamide (Compound 143)

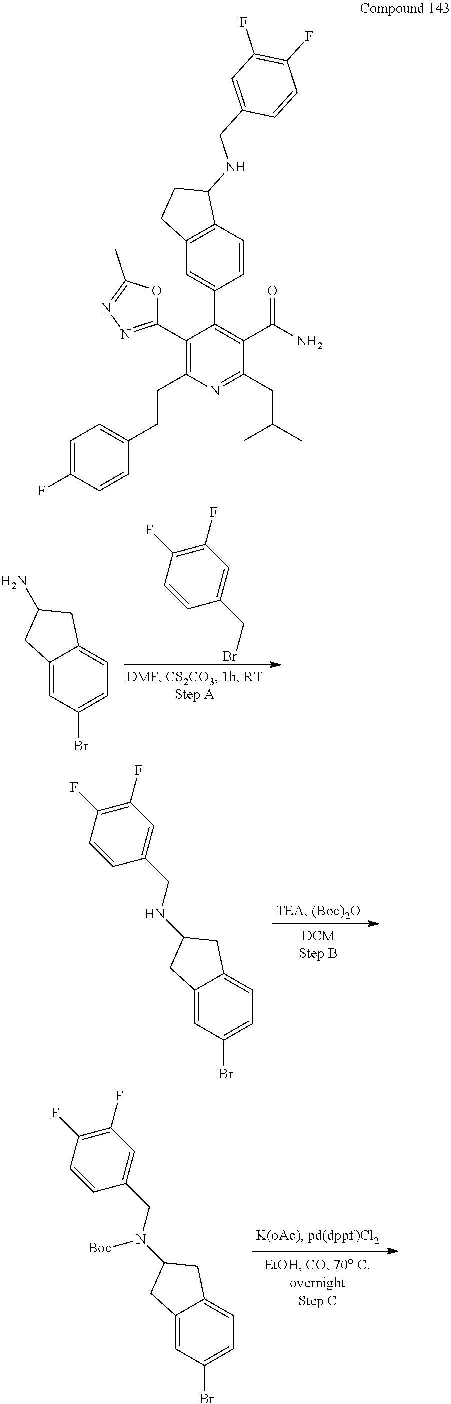

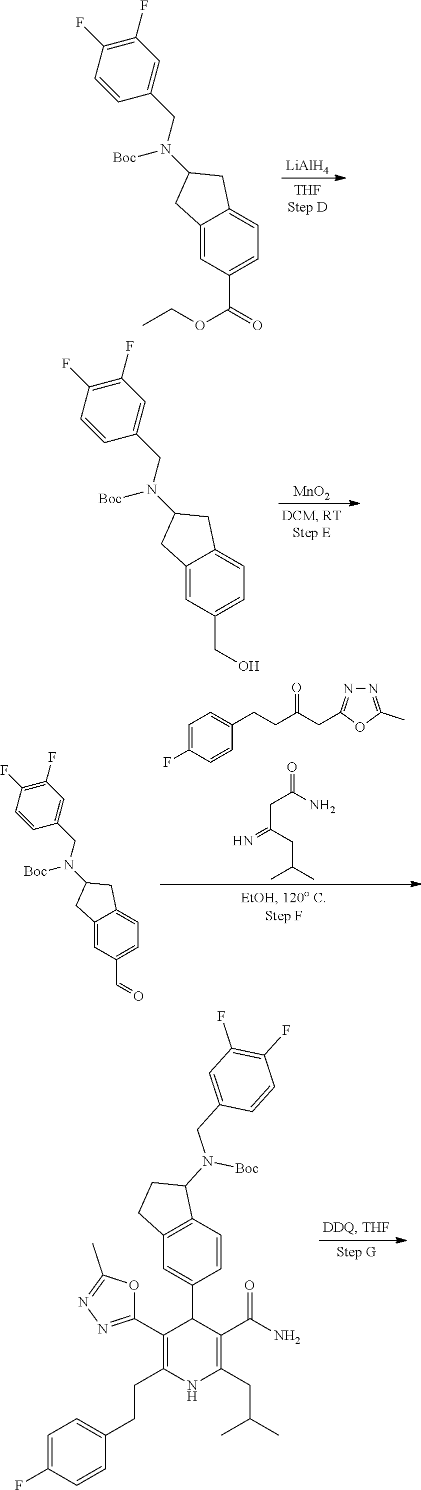

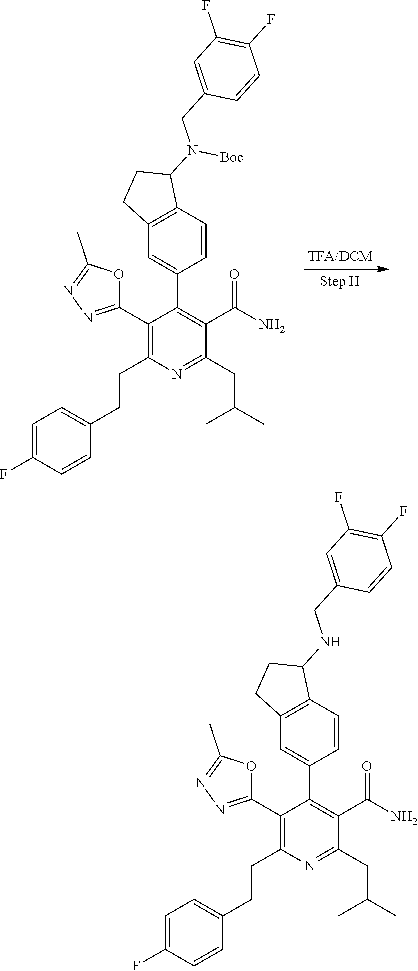 [SMILES: Cc1nnc(-c2c(CCc3ccc(F)cc3)nc(CC(C)C)c(C(N)=O)c2-c2ccc3c(c2)CCC3N(Cc2ccc(F)c(F)c2)C(=O)OC(C)(C)C)o1 ; Cc1nnc(-c2c(CCc3ccc(F)cc3)nc(CC(C)C)c(C(N)=O)c2-c2ccc3c(c2)CCC3NCc2ccc(F)c(F)c2)o1]

### Step A 5-bromo-N-(3,4-difluorobenzyl)-2,3-dihydro-1H-inden-1-amine

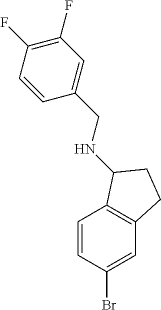 [SMILES: Fc1ccc(CNC2CCc3cc(Br)ccc32)cc1F]

To a mixture of 5-bromo-2,3-dihydro-1H-inden-1-amine (1 g, 4.715 mmol) in DMF (10 mL) was added Cs₂CO₃ (4.61 g, 14.145 mmol) and 4-(bromomethyl)-1,2-difluorobenzene (0.98 g, 4.715 mmol) at room temperature. The mixture was stirred at room temperature for 1 hr. After the reaction was completed, the reaction mixture was quenched with H₂O (25 mL) and extracted with ethyl acetate (20 mL×3). The organic layer was combined and washed with brine (20 mL×2), dried over sodium sulfate, filtered and concentrated in vacuum. The reaction mixture was purified by column chromatography (PE/EtOAc=10/1) to give 5-bromo-N-(3,4-difluorobenzyl)-2,3-dihydro-1H-inden-1-amine (1.2 g, crude). LC-MS: m/z 338.0 (M+H)⁺.

### Step B tert-butyl (5-bromo-2,3-dihydro-1H-inden-1-yl)(3,4-difluorobenzyl)carbamate

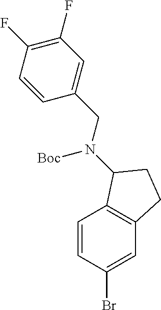 [SMILES: CC(C)(C)OC(=O)N(Cc1ccc(F)c(F)c1)C1CCc2cc(Br)ccc21]

To a mixture of 5-bromo-N-(3,4-difluorobenzyl)-2,3-dihydro-1H-inden-1-amine (1.2 g, crude) in DCM (12.0 mL) was added (Boc)₂O (1.55 g, 7.096 mmol) and TEA (0.5 mL) at room temperature. The mixture was stirred at room temperature for 3 hrs. The reaction mixture was quenched with H₂O (200 mL) and extracted with ethyl acetate (100 mL×3). The organic layer was combined and washed with brine (100 mL×2), dried over Na₂SO₄, filtered and concentrated in vacuum. The reaction mixture was purified by column chromatography (PE/EtOAc=20/1) to give tert-butyl (5-bromo-2,3-dihydro-1H-inden-1-yl)(3,4-difluorobenzyl)carbamate (1.4 g, 90.02%). LC-MS: m/z 381.8 (M+H-tBu)⁺.

### Step C ethyl 1-((tert-butoxycarbonyl)(3,4-difluorobenzyl)amino)-2,3-dihydro-1H-indene-5-carboxylate

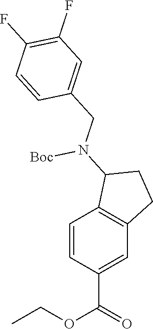 [SMILES: CCOC(=O)c1ccc2c(c1)CCC2N(Cc1ccc(F)c(F)c1)C(=O)OC(C)(C)C]

To a mixture of tert-butyl (5-bromo-2,3-dihydro-1H-inden-1-yl)(3,4-difluorobenzyl)carbamate (800 mg, 1.825 mmol) in EtOH (15.0 mL) was added potassium acetate (358.21 mg, 3.65 mmol) and bis[5-(diphenylphosphanyl)cyclopenta-1,3-dienyl]-λ2-iron(II) dichloromethane palladium chloride (149.04 mg, 0.183 mmol) at room temperature. The mixture was stirred at 70° C. overnight under N₂. The reaction mixture was cooled down to room temperature, quenched with H₂O (50 mL) and extracted with EtOAc (50 mL×3). The organic layer was combined and washed with brine (50 mL×2), dried over Na₂SO₄, filtered and concentrated in vacuum. The reaction mixture was purified by column chromatography (PE/EtOAc=10/1) to give ethyl 1-((tert-butoxycarbonyl)(3,4-difluorobenzyl)amino)-2,3-dihydro-1H-indene-5-carboxylate (700 mg, 88.89%). LC-MS: m/z 376.1 (M+H-tBu)⁺.

### Step D tert-butyl (3,4-difluorobenzyl)(5-(hydroxymethyl)-2,3-dihydro-1H-inden-1-yl)carbamate

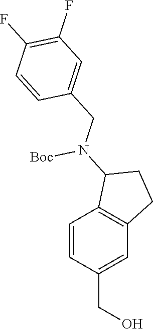 [SMILES: CC(C)(C)OC(=O)N(Cc1ccc(F)c(F)c1)C1CCc2cc(CO)ccc21]

To a solution of ethyl 1-((tert-butoxycarbonyl)(3,4-difluorobenzyl)amino)-2,3-dihydro-1H-indene-5-carboxylate (700 mg, 1.622 mmol) in THF (10.0 mL) was added LiAlH₄ (123.13 mg, 3.245 mmol) at 0° C. The mixture was stirred at room temperature for 1 hour. To the reaction mixture was added EtOAc (20 mL) and quenched with H₂O (1 mL), the reaction mixture was further stirred at room temperature for 10 min, filtered and concentrated in vacuum. The reaction mixture was purified by column chromatography (PE/EtOAc=3/1) to give tert-butyl (3,4-difluorobenzyl)(5-(hydroxymethyl)-2,3-dihydro-1H-inden-1-yl)carbamate (600 mg, 94.97%). LC-MS: m/z 334.1 (M+H-tBu)⁺.

### Step E tert-butyl (3,4-difluorobenzyl)(5-formyl-2,3-dihydro-1H-inden-1-yl)carbamate

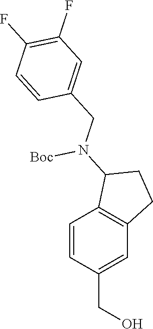 [SMILES: CC(C)(C)OC(=O)N(Cc1ccc(F)c(F)c1)C1CCc2cc(CO)ccc21]

To a solution of tert-butyl (3,4-difluorobenzyl)(5-(hydroxymethyl)-2,3-dihydro-1H-inden-1-yl)carbamate (150 mg, 0.385 mmol) in dioxane (5.0 mL) was added MnO₂ (200.92 mg, 2.311 mmol) at room temperature. The solution was stirred at 70° C. overnight. The reaction mixture was cooled down to room temperature, filtered and concentrated in vacuum. The reaction mixture was purified by column chromatography (PE/EtOAc=10/1) to give tert-butyl (3,4-difluorobenzyl)(5-formyl-2,3-dihydro-1H-inden-1-yl)carbamate (100 mg, 67.01%). LC-MS: m/z 332.0 (M+H-tBu)⁺.

### Step F tert-butyl (5-(3-carbamoyl-6-(4-fluorophenethyl)-2-isobutyl-5-(5-methyl-1,3,4-oxadiazol-2-yl)-1,4-dihydropyridin-4-yl)-2,3-dihydro-1H-inden-1-yl)(3,4-difluorobenzyl)carbamate

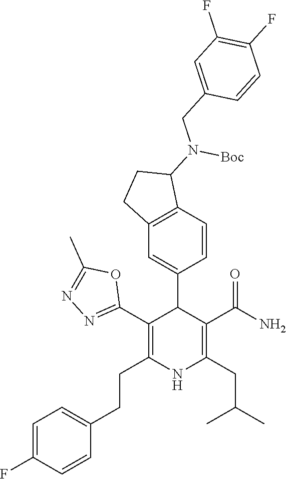 [SMILES: Cc1nnc(C2=C(CCc3ccc(F)cc3)NC(CC(C)C)=C(C(N)=O)C2c2ccc3c(c2)CCC3N(Cc2ccc(F)c(F)c2)C(=O)OC(C)(C)C)o1]

A mixture of tert-butyl (3,4-difluorobenzyl)(5-formyl-2,3-dihydro-1H-inden-1-yl)carbamate (50 mg, 0.129 mmol), 4-(4-fluorophenyl)-1-(5-methyl-1,3,4-oxadiazol-2-yl)butan-2-one (18.35 mg, 0.129 mmol), 3-imino-5-methylhexanamide (32.04 mg, 0.129 mmol) in EtOH (2.0 mL) was stirred at 120° C. overnight. The reaction mixture was cooled down to room temperature, concentrated in vacuum and purified by Prep-TLC (PE/EtOAc=1/1) to give tert-butyl (5-(3-carbamoyl-6-(4-fluorophenethyl)-2-isobutyl-5-(5-methyl-1,3,4-oxadiazol-2-yl)-1,4-dihydropyridin-4-yl)-2,3-dihydro-1H-inden-1-yl)(3,4-difluorobenzyl)carbamate (60 mg, 62.67%). LC-MS: m/z 742.3 (M+H)⁺.

### Step G tert-butyl (5-(3-carbamoyl-6-(4-fluorophenethyl)-2-isobutyl-5-(5-methyl-1,3,4-oxadiazol-2-yl)pyridin-4-yl)-2,3-dihydro-1H-inden-1-yl)(3,4-difluorobenzyl)carbamate

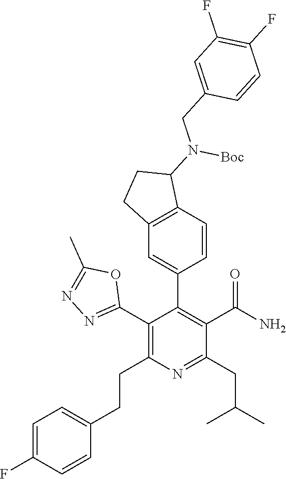 [SMILES: Cc1nnc(-c2c(CCc3ccc(F)cc3)nc(CC(C)C)c(C(N)=O)c2-c2ccc3c(c2)CCC3N(Cc2ccc(F)c(F)c2)C(=O)OC(C)(C)C)o1]

To a mixture of tert-butyl (5-(3-carbamoyl-6-(4-fluorophenethyl)-2-isobutyl-5-(5-methyl-1,3,4-oxadiazol-2-yl)-1,4-dihydropyridin-4-yl)-2,3-dihydro-1H-inden-1-yl)(3,4-difluorobenzyl)carbamate (50 mg, 0.067 mmol) in THF (2.0 mL) was added DDQ (22.95 mg, 0.101 mmol) at room temperature. The mixture was stirred at room temperature for 1 hr. The reaction mixture was quenched with aq. NaHCO₃ solution (30 mL) and extracted with EtOAc (20 mL×3). The organic layer was combined and washed with brine (50 mL×2), dried over Na₂SO₄, filtered and concentrated in vacuum to give tert-butyl (5-(3-carbamoyl-6-(4-fluorophenethyl)-2-isobutyl-5-(5-methyl-1,3,4-oxadiazol-2-yl)pyridin-4-yl)-2,3-dihydro-1H-inden-1-yl)(3,4-difluorobenzyl)carbamate (60 mg, crude). LC-MS: m/z 740.2 (M+H)⁺.

### Step H 4-(1-{[(3,4-difluorophenyl)methyl]amino}-5-indanyl)-6-[2-(p-fluorophenyl)ethyl]-2-isobutyl-5-(5-methyl-1,3,4-oxadiazol-2-yl)nicotinamide (Compound 143)

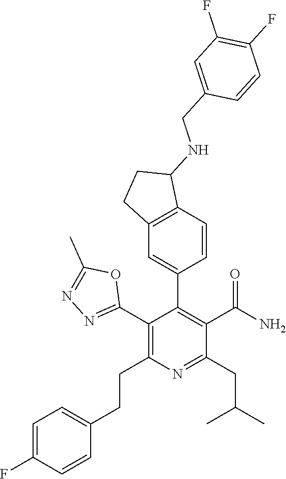 [SMILES: Cc1nnc(-c2c(CCc3ccc(F)cc3)nc(CC(C)C)c(C(N)=O)c2-c2ccc3c(c2)CCC3NCc2ccc(F)c(F)c2)o1]

To a solution of tert-butyl (5-(3-carbamoyl-6-(4-fluorophenethyl)-2-isobutyl-5-(5-methyl-1,3,4-oxadiazol-2-yl)pyridin-4-yl)-2,3-dihydro-1H-inden-1-yl)(3,4-difluorobenzyl)carbamate (50 mg, crude) in DCM (5.0 mL) was added TFA (1.0 mL) at room temperature. The solution was stirred at room temperature for 1 hr. The reaction mixture was quenched with aq. NaHCO₃ solution (30 mL) and extracted with EtOAc (20 mL×3). The organic layer was combined and washed with brine (50 mL×2), dried over Na₂SO₄, filtered and concentrated in vacuum. The reaction mixture was purified by Prep-HPLC to give 4-(1-{[(3,4-difluorophenyl)methyl]amino}-5-indanyl)-6-[2-(p-fluorophenyl)ethyl]-2-isobutyl-5-(5-methyl-1,3,4-oxadiazol-2-yl)nicotinamide (13.02 mg, 30.23%).

¹H NMR (400 MHz, DMSO-d₆) δ 7.73 (br s, 1H), 7.31-7.50 (m, 3H), 7.27 (d, J=7.6 Hz, 1H), 7.19-7.25 (m, 1H), 7.01-7.13 (m, 5H), 6.98 (br s, 1H), 6.92 (d, J=8.0 Hz, 1H), 4.08 (t, J=6.8 Hz, 1H), 3.74 (s, 2H), 2.96 (s, 3H), 2.77-2.87 (m, 1H), 2.72 (d, J=6.8 Hz, 2H), 2.58-2.69 (m, 2H), 2.26-2.35 (m, 2H), 2.24 (s, 3H), 1.67-1.79 (m, 1H), 0.94 (d, J=6.8 Hz, 6H). ¹⁹F NMR (377 MHz, DMSO-d₆) δ −117.35, −139.39, −142.10. LC-MS: m/z 640.2 (M+H)⁺.

### Example 20

### 4-(1-(3,4-difluorophenethyl)-1H-indol-5-yl)-6-(4-fluorophenethyl)-2-isobutyl-5-(5-methyl-1,3,4-oxadiazol-2-yl)nicotinamide (Compound 144)

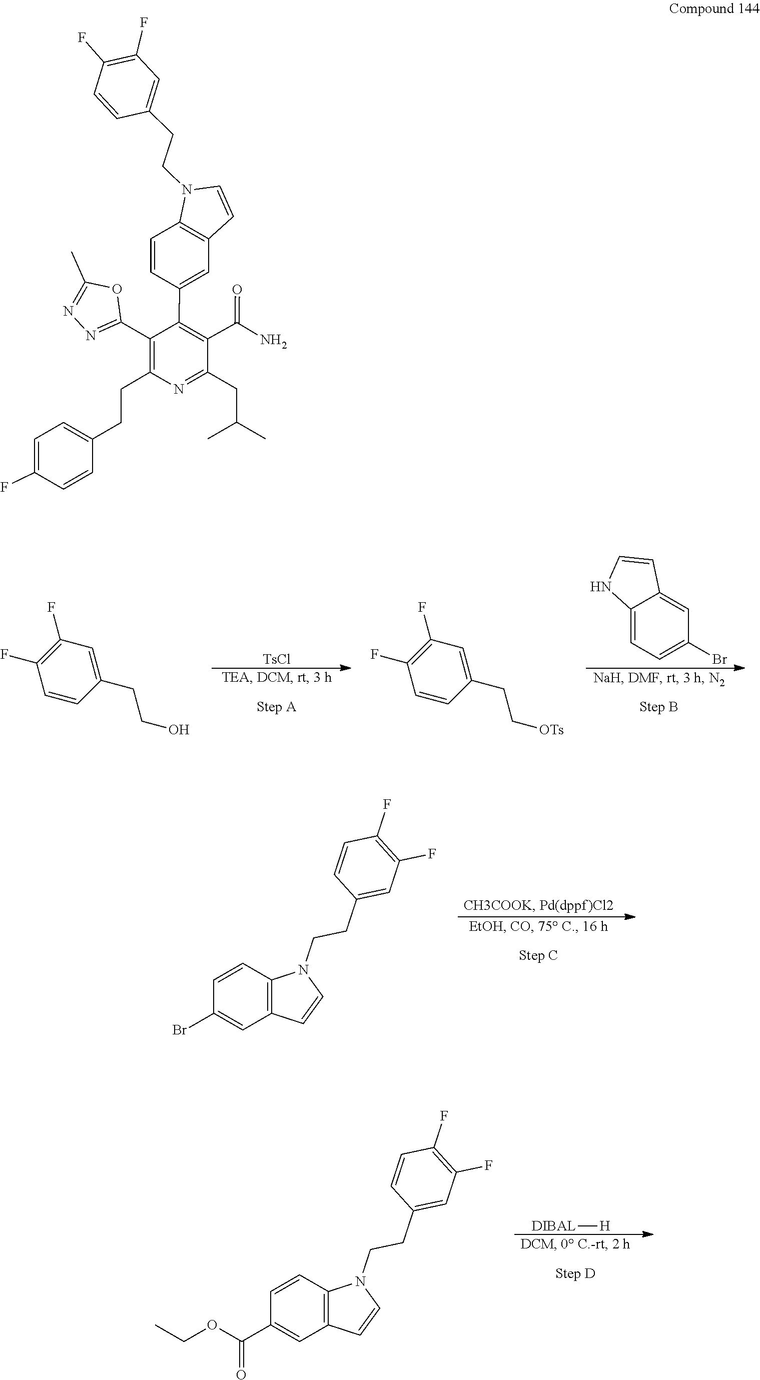

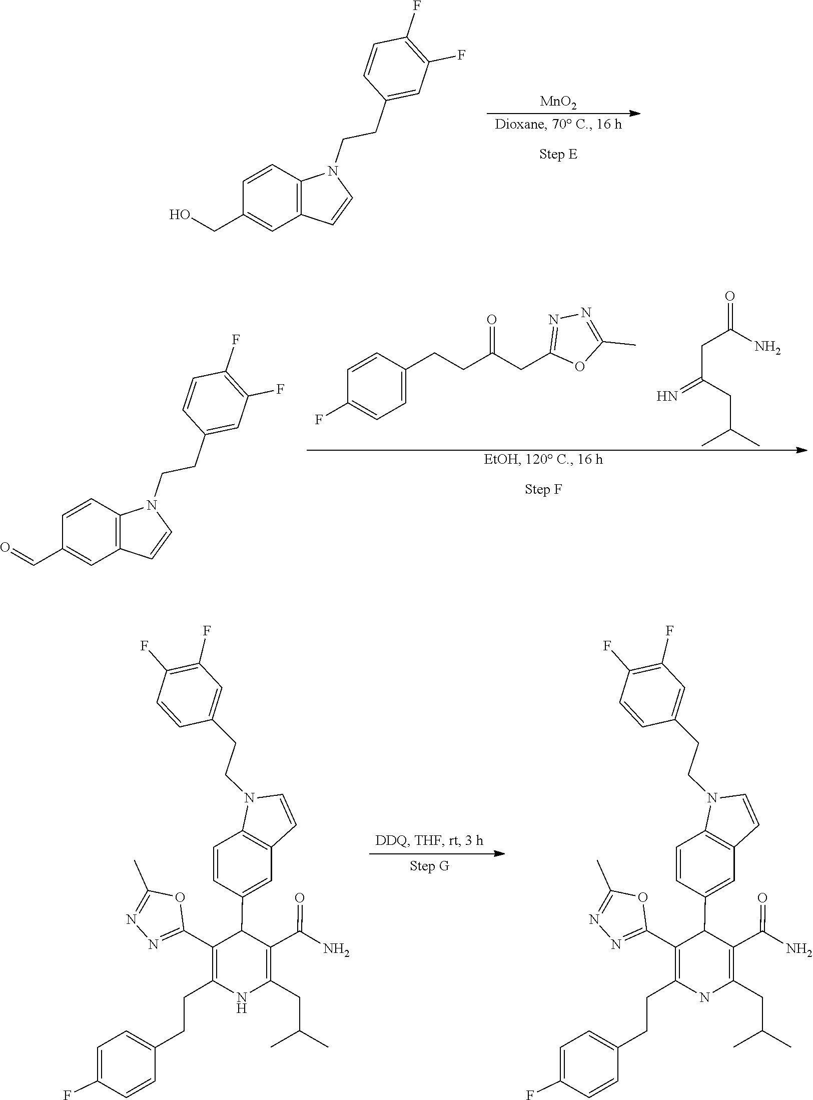

### Step A 3,4-difluorophenethyl 4-methylbenzenesulfonate

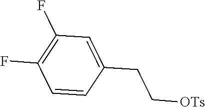 [SMILES: Cc1ccc(S(=O)(=O)OCCc2ccc(F)c(F)c2)cc1]

To a solution of 2-(3,4-difluorophenyl)ethan-1-ol (720 mg, 4.56 mmol) in DCM (7 mL) was added TsCl (1.13 g, 5.93 mmol) and TEA (1 ml). The solution was stirred at room temperature for 3 hour. The mixture was quenched with H₂O (50 mL), extracted with EtOAc (50 mL*2). The combined organic layers were washed with brine (50 mL), dried over anhydrous Na₂SO₄, filtered and concentrated. The residue was purified by column (PE/EA=5/1) to give 3,4-difluorophenethyl 4-methylbenzenesulfonate (1.18 g, 82.7%).

### Step B 5-bromo-1-(3,4-difluorophenethyl)-1H-indole

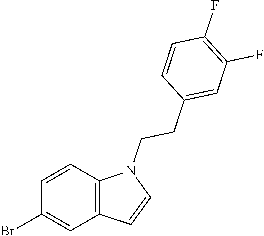 [SMILES: Fc1ccc(CCn2ccc3cc(Br)ccc32)cc1F]

To a solution of 5-bromo-1H-indole (618 mg, 3.17 mmol) and 3,4-difluorophenethyl 4-methylbenzenesulfonate (1.18 g, 3.80 mmol) in DMF (15 mL) was added NaH (190 mg, 4.76 mmol) at 0° C. under N₂. The solution was stirred at room temperature for 3 hour. The mixture was quenched with H₂O (80 mL), extracted with EtOAc (80 mL*2). The combined organic layers were washed with brine (100 mL), dried over anhydrous Na₂SO₄, filtered and concentrated. The residue was purified by column (PE/EA=8/1) to give 5-bromo-1-(3,4-difluorophenethyl)-1H-indole (460 mg, yield: 44.1%). LC-MS: m/z 336.0 (M+H)⁺.

### Step C ethyl 1-(3,4-difluorophenethyl)-1H-indole-5-carboxylate

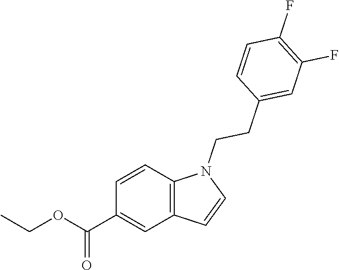 [SMILES: CCOC(=O)c1ccc2c(ccn2CCc2ccc(F)c(F)c2)c1]

A mixture of 5-bromo-1-(3,4-difluorophenethyl)-1H-indole (460 mg, 1.37 mmol), CH₃COOK (540 mg, 5.49 mmol) and Pd(dppf)Cl₂ (102 mg, 0.14 mmol) in EtOH (5 mL) was stirred at 75° C. for 16 hours under CO. The mixture was filtered and concentrated under reduced pressure. The residue was purified by silica gel column chromatography (PE/EA=3/1) to give ethyl 1-(3,4-difluorophenethyl)-1H-indole-5-carboxylate (167 mg, yield: 37.0%). LC-MS: m/z 330.1 (M+H)⁺.

### Step D (1-(3,4-difluorophenethyl)-1H-indol-5-yl)methanol

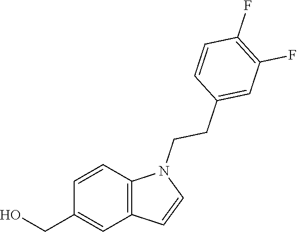 [SMILES: OCc1ccc2c(ccn2CCc2ccc(F)c(F)c2)c1]

To a solution of ethyl 1-(3,4-difluorophenethyl)-1H-indole-5-carboxylate (117 mg, 0.36 mmol) in DCM (4 mL) was added DIBAL-H (0.5 ml, 0.53 mmol) at 0° C. under N₂. The solution was stirred at room temperature for 2 hour. The mixture was quenched with H₂O (30 mL), extracted with EtOAc (30 mL*2). The combined organic layers were washed with brine (50 mL), dried over anhydrous Na₂SO₄, filtered and concentrated. The residue was purified by column (PE/EA=3/1) to give (1-(3,4-difluorophenethyl)-1H-indol-5-yl)methanol (62 mg, yield: 60.8%). LC-MS: m/z 288.0 (M+H)⁺.

### Step E 1-(3,4-difluorophenethyl)-1H-indole-5-carbaldehyde

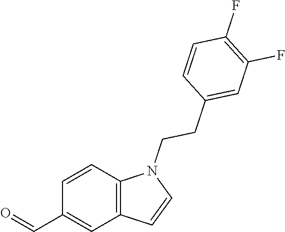 [SMILES: O=Cc1ccc2c(ccn2CCc2ccc(F)c(F)c2)c1]

To a solution of (1-(3,4-difluorophenethyl)-1H-indol-5-yl)methanol (62 mg, 0.22 mmol) in dioxane (2 mL) was added MnO₂ (150 mg, 1.73 mmol) and stirred at 70° C. for 16 hour. The mixture was filtered and concentrated. The residue was purified by column (PE/EA=3/1) to give 1-(3,4-difluorophenethyl)-1H-indole-5-carbaldehyde (56 mg, yield: 91.8%). LC-MS: m/z 286.1 (M+H)⁺.

### Step F 4-(1-(3,4-difluorophenethyl)-1H-indol-5-yl)-6-(4-fluorophenethyl)-2-isobutyl-5-(5-methyl-1,3,4-oxadiazol-2-yl)-1,4-dihydropyridine-3-carboxamide

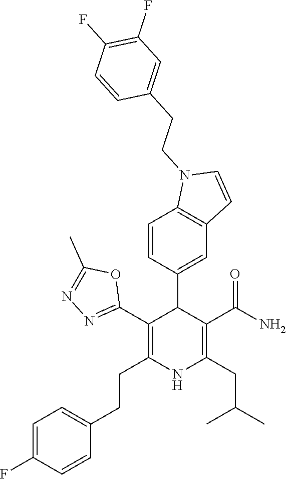 [SMILES: Cc1nnc(C2=C(CCc3ccc(F)cc3)NC(CC(C)C)=C(C(N)=O)C2c2ccc3c(ccn3CCc3ccc(F)c(F)c3)c2)o1]

A mixture of 1-(3,4-difluorophenethyl)-1H-indole-5-carbaldehyde (56 mg, 0.20 mmol), 4-(4-fluorophenyl)-1-(5-methyl-1,3,4-oxadiazol-2-yl)butan-2-one (202 mg, 0.20 mmol) and 3-imino-5-methylhexanamide (33 mg, 0.24 mmol) in EtOH (10 mL) was stirred at 120° C. for 16 hours in seal tube. The mixture was concentrated under reduced pressure. The residue was purified by prep-TLC to give 4-(1-(3,4-difluorophenethyl)-1H-indol-5-yl)-6-(4-fluorophenethyl)-2-isobutyl-5-(5-methyl-1,3,4-oxadiazol-2-yl)-1,4-dihydropyridine-3-carboxamide (38 mg, yield: 30.4%). LC-MS: m/z 640.2 (M+H)⁺.

### Step G 4-(1-(3,4-difluorophenethyl)-1H-indol-5-yl)-6-(4-fluorophenethyl)-2-isobutyl-5-(5-methyl-1,3,4-oxadiazol-2-yl)nicotinamide (Compound 144)

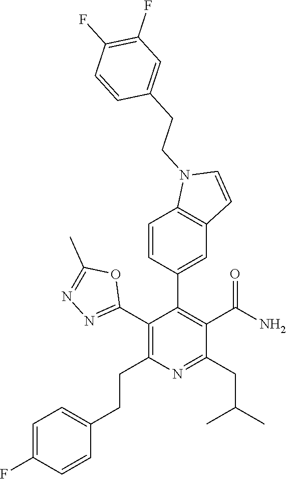 [SMILES: Cc1nnc(-c2c(CCc3ccc(F)cc3)nc(CC(C)C)c(C(N)=O)c2-c2ccc3c(ccn3CCc3ccc(F)c(F)c3)c2)o1]

To a mixture of 4-(1-(3,4-difluorophenethyl)-1H-indol-5-yl)-6-(4-fluorophenethyl)-2-isobutyl-5-(5-methyl-1,3,4-oxadiazol-2-yl)-1,4-dihydropyridine-3-carboxamide (38 mg, 0.06 mmol) in THF (1 mL) was added DDQ (27 mg, 0.12 mmol). The solution was stirred at room temperature for 3 hrs. The solution was filtered and the filtrate was purified by Prep-HPLC (0.1% NH₃H₂O/H₂O/CH₃CN) to give 4-(1-(3,4-difluorophenethyl)-1H-indol-5-yl)-6-(4-fluorophenethyl)-2-isobutyl-5-(5-methyl-1,3,4-oxadiazol-2-yl)nicotinamide (7.71 mg, 20.3%).

¹H NMR (400 MHz, DMSO-d₆) δ 7.64 (br s, 1H), 7.43 (d, J=8.4 Hz, 1H), 7.25-7.37 (m, 5H), 6.97-7.14 (m, 5H), 6.87 (dd, J=1.2, 8.4 Hz, 1H), 6.34 (d, J=2.8 Hz, 1H), 4.37 (t, J=7.2 Hz, 2H), 3.05 (t, J=7.2 Hz, 2H), 2.91-2.30 (m, 4H), 2.73 (d, J=7.2 Hz, 2H), 2.26-2.38 (m, 1H), 2.16 (s, 3H), 0.95 (d, J=6.4 Hz, 6H). 19F NMR (377 MHz, DMSO-d₆) δ −117.37, −139.23, −142.15. ¹⁹F NMR (377 MHz, DMSO-d6): δ −117.51. LC-MS: m/z 638.1 (M+H)⁺.

### Example 21

### (3,4-difluorophenyl)methyl 2-{5-carbamoyl-2-[2-(p-fluorophenyl)ethyl]-6-isobutyl-3-(5-methyl-1,3,4-oxadiazol-2-yl)-4-pyridyl}ethanecarbamate (Compound 145)

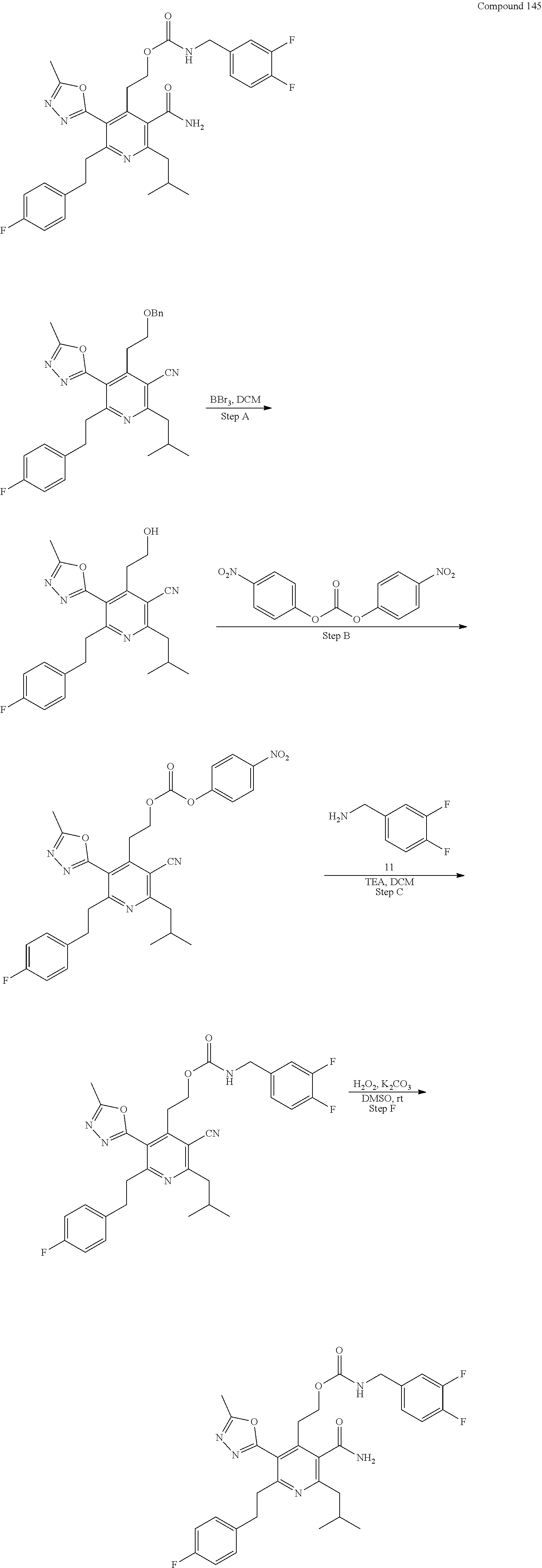

### Step A 6-(4-fluorophenethyl)-4-(2-hydroxyethyl)-2-isobutyl-5-(5-methyl-1,3,4-oxadia-zol-2-yl)nicotinonitrile

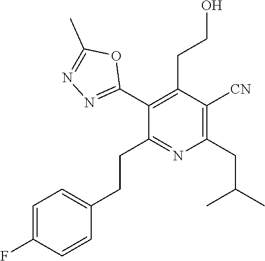 [SMILES: Cc1nnc(-c2c(CCc3ccc(F)cc3)nc(CC(C)C)c(C#N)c2CCO)o1]

To a solution of 4-(2-benzyloxyethyl)-6-[2-(4-fluorophenyl)ethyl]-2-isobutyl-5-(5-methyl-1,3,4-oxadiazol-2-yl)pyridine-3-carbonitrile (130 mg, 260.74 μmol, synthesized by using a similar procedure described in Example 4) in DCM (5 mL) was added BBr₃ (1 M, 2.61 mL) by dropwise at 0° C. for 3 min. The mixture was stirred at 22° C. for 16 hrs. The reaction mixture was quenched by addition water (20 mL) at 0° C., and then extracted with DCM (20 mL×2). The combined organic layers were washed with brine (10 mL), dried over Na₂SO₄, filtered and concentrated under reduced pressure to give a residue. The residue was purified by prep-TLC (SiO₂, DCM:MeOH=10:1) to give 6-(4-fluorophenethyl)-4-(2-hydroxyethyl)-2-isobutyl-5-(5-methyl-1,3,4-oxadia-zol-2-yl)nicotinonitrile (58 mg, 54.5%). ¹H NMR (400 MHz, CDCl₃) δ 7.12-7.20 (m, 2H), 6.98-7.06 (m, 2H), 4.32 (t, J=5.62 Hz, 2H), 3.66 (dd, J=8.68, 6.85 Hz, 2H), 3.15-3.21 (m, 2H), 3.05-3.12 (m, 2H), 2.89 (d, J=7.21 Hz, 2H), 2.21-2.29 (m, 1H), 2.17 (s, 3H), 0.97 (d, J=6.60 Hz, 6H). LC-MS: m/z 409.4 (M+H)⁺.

### Step B 2-(3-cyano-6-(4-fluorophenethyl)-2-isobutyl-5-(5-methyl-1,3,4-oxadiazol-2-yl)pyridin-4-yl)ethyl (4-nitrophenyl) carbonate

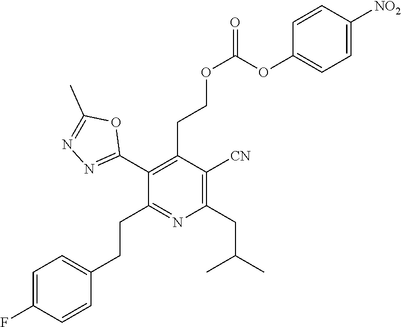 [SMILES: Cc1nnc(-c2c(CCc3ccc(F)cc3)nc(CC(C)C)c(C#N)c2CCOC(=O)Oc2ccc([N+](=O)[O-])cc2)o1]

To a solution of bis(4-nitrophenyl) carbonate (47.52 mg, 156.19 μmol) in DCM (5 mL) was added TEA (43.10 mg, 425.98 μmol) and 6-[2-(4-fluorophenyl)ethyl]-4-(2-hydroxyethyl)-2-isobutyl-5-(5-methyl-1,3,4-oxadiazol-2-yl)pyridine-3-carbonitrile (58 mg, 141.99 μmol). The mixture was stirred at 22° C. for 16 hrs. The reaction mixture was concentrated under reduced pressure to remove solvent to give 2-(3-cyano-6-(4-fluorophenethyl)-2-isobutyl-5-(5-methyl-1,3,4-oxadiazol-2-yl)pyridin-4-yl)ethyl (4-nitrophenyl) carbonate (200 mg, crude). LC-MS: m/z 574.4 (M+H)⁺.

### Step C 2-(3-cyano-6-(4-fluorophenethyl)-2-isobutyl-5-(5-methyl-1,3,4-oxadiazol-2-yl)pyridin-4-yl)ethyl (3,4-difluorobenzyl)carbamate

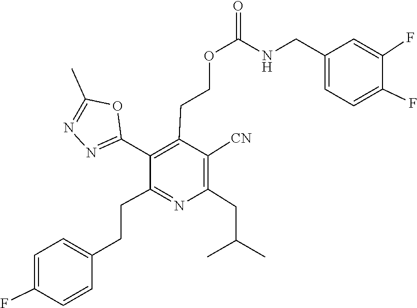 [SMILES: Cc1nnc(-c2c(CCc3ccc(F)cc3)nc(CC(C)C)c(C#N)c2CCOC(=O)NCc2ccc(F)c(F)c2)o1]

A mixture of (3,4-difluorophenyl)methanamine (15 mg, 104.61 μmol), 2-[5-cyano-2-[2-(4-fluorophenyl)ethyl]-6-isobutyl-3-(5-methyl-1,3,4-oxadiazol-2-yl)-4-pyridyl]ethyl (4-nitrophenyl) carbonate (50 mg, 87.17 μmol), TEA (26.6 mg, 261.52 μmol) in DCM (4 mL) was degassed and purged with N₂ for 3 times. Then the mixture was stirred at 22° C. for 5 hrs under N₂ atmosphere. The reaction mixture was concentrated under reduced pressure to remove solvent. The residue was purified by prep-TLC (SiO₂, PE/EA=3/1) to give 2-(3-cyano-6-(4-fluorophenethyl)-2-isobutyl-5-(5-methyl-1,3,4-oxadiazol-2-yl)pyridin-4-yl)ethyl (3,4-difluorobenzyl)carbamate (28 mg, 55.61%).

¹H NMR (400 MHz, CDCl₃) δ 8.17 (d, J=9.20 Hz, 1H), 7.03-7.18 (m, 2H), 6.97-6.98 (m, 2H), 6.85-6.95 (m, 3H), 4.16-4.32 (m, 4H), 2.91-3.13 (m, 8H), 2.60 (s, 3H), 2.21-2.29 (m, 1H), 1.01 (d, J=6.60 Hz, 6H). LC-MS: m/z 578.6 (M+H)⁺.

### Step D (3,4-difluorophenyl)methyl 2-{5-carbamoyl-2-[2-(p-fluorophenyl)ethyl]-6-isobutyl-3-(5-methyl-1,3,4-oxadiazol-2-yl)-4-pyridyl}ethanecarbamate (Compound 145)

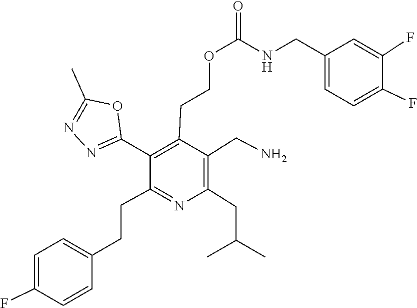 [SMILES: Cc1nnc(-c2c(CCc3ccc(F)cc3)nc(CC(C)C)c(CN)c2CCOC(=O)NCc2ccc(F)c(F)c2)o1]

To a solution of 2-(3-cyano-6-(4-fluorophenethyl)-2-isobutyl-5-(5-methyl-1,3,4-oxadiazol-2-yl)pyridin-4-yl)ethyl (3,4-difluorobenzyl)carbamate (28 mg, 48.48 μmol) in DMSO (2 mL) was added K₂CO₃ (3.5 mg, 24.24 μmol). Then H₂O₂ (0.01 g, 97.03 μmol, 33% purity) was added dropwise at 0° C. The resulting mixture was stirred at 22° C. for 16 hrs. The residue was diluted with water (30 mL) and extracted with EA (30 mL×2). The combined organic layers were washed with brine (30 mL), dried over Na₂SO₄, filtered and concentrated under reduced pressure to give a residue. The crude product was purified by prep-HPLC (0.1% FA condition) to give (3,4-difluorophenyl)methyl 2-{5-carbamoyl-2-[2-(p-fluorophenyl)ethyl]-6-isobutyl-3-(5-methyl-1,3,4-oxadiazol-2-yl)-4-pyridyl}ethanecarbamate (1.11 mg, 3.84%).

¹H NMR (400 MHz, CDCl₃) δ 6.95-7.08 (m, 5H), 6.90 (t, J=8.68 Hz, 3H), 6.33 (s, 1H), 5.93 (s, 1H), 4.25 (s, 2H), 4.14 (d, J=5.87 Hz, 2H), 2.90-2.98 (m, 6H), 2.78 (d, J=7.34 Hz, 2H), 2.59 (s, 3H), 2.35 (d, J=6.60 Hz, 1H), 0.96 (d, J=6.85 Hz, 6H). LC-MS: m/z 596.2 (M+H)⁺.

### Example 22

### 4-[5-[(3,4-difluorophenyl)methylcarbamoyl]-2-thienyl]-6-[(4-fluorophenyl)methoxy]-2-isobutyl-5-(5-methyl-1,3,4-oxadiazol-2-yl)pyridine-3-carboxamide (Compound 146)

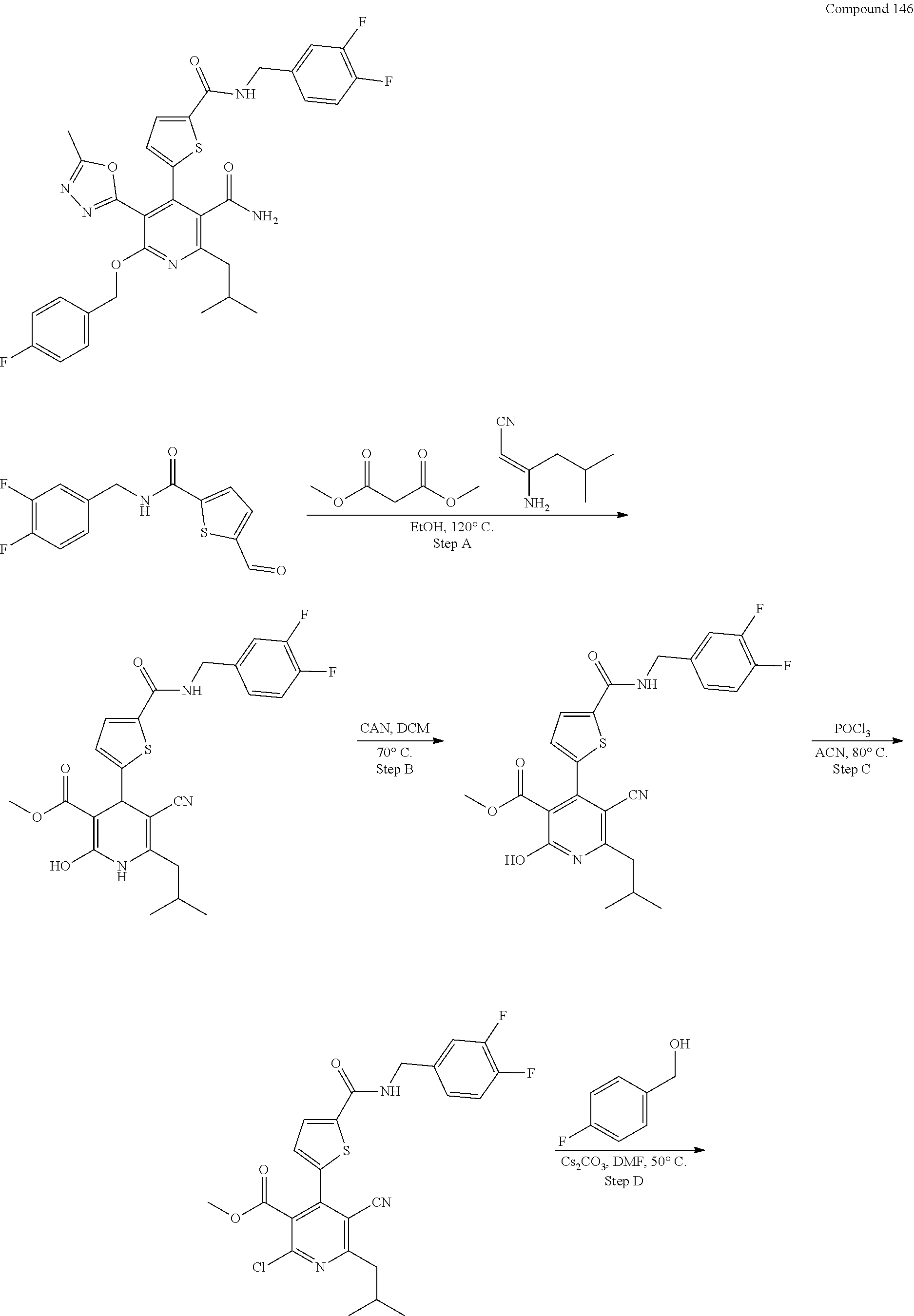

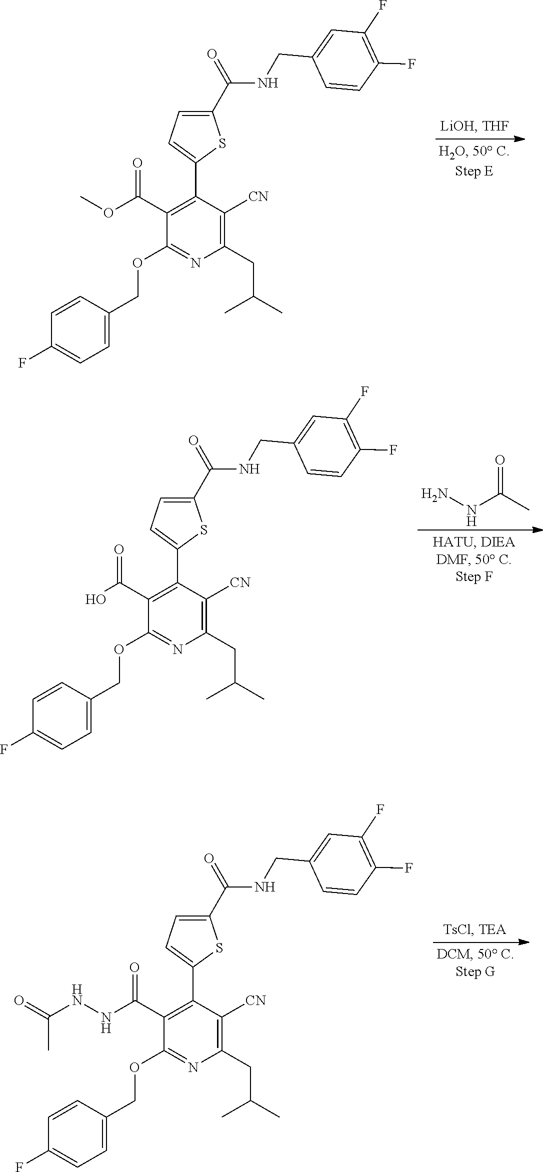

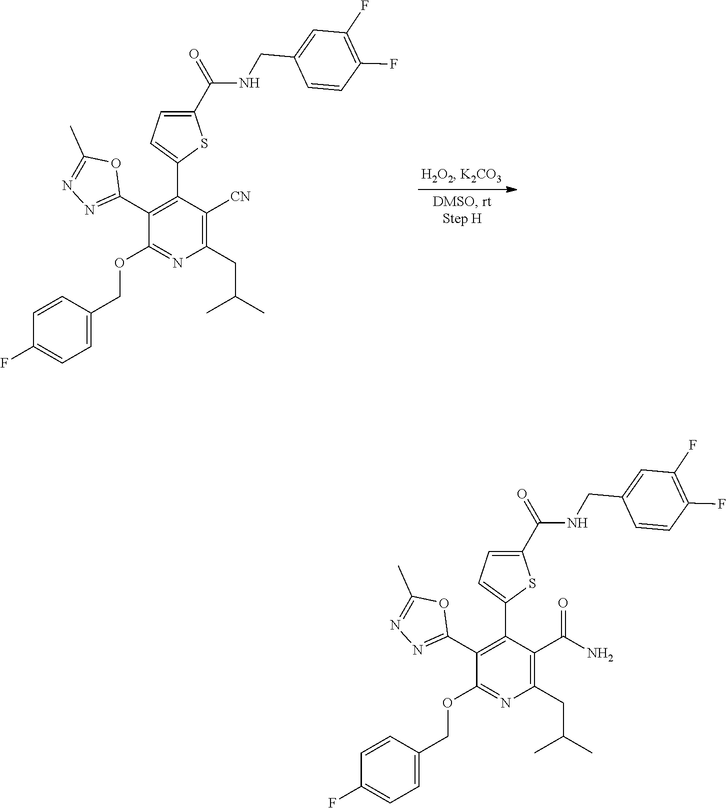 [SMILES: Cc1nnc(-c2c(OCc3ccc(F)cc3)nc(CC(C)C)c(C#N)c2-c2ccc(C(=O)NCc3ccc(F)c(F)c3)s2)o1 ; Cc1nnc(-c2c(OCc3ccc(F)cc3)nc(CC(C)C)c(C(N)=O)c2-c2ccc(C(=O)NCc3ccc(F)c(F)c3)s2)o1]

### Step A methyl 5-cyano-4-(5-((3,4-difluorobenzyl)carbamoyl)thiophen-2-yl)-2-hydroxy-6-isobutyl-1,4-dihydropyridine-3-carboxylate

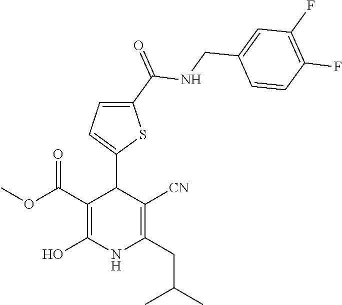 [SMILES: COC(=O)C1=C(O)NC(CC(C)C)=C(C#N)C1c1ccc(C(=O)NCc2ccc(F)c(F)c2)s1]

To a solution of N-[(3,4-difluorophenyl)methyl]-5-formyl-thiophene-2-carboxamide (1 g, 3.56 mmol) in EtOH (30 mL) was added dimethyl propanedioate (469.69 mg, 3.56 mmol) and (Z)-3-amino-5-methyl-hex-2-enenitrile (441.50 mg, 3.56 mmol). The mixture was stirred at 120° C. for 8 hrs. The reaction mixture was concentrated under reduced pressure to give crude methyl 5-cyano-4-(5-((3,4-difluorobenzyl)carbamoyl)thiophen-2-yl)-2-hydroxy-6-isobutyl-1,4-dihydropyridine-3-carboxylate (1.5 g, crude), which was used into the next step without further purification. LC-MS: m/z 486.3 (M−H)⁻.

### Step B methyl 5-cyano-4-[5-[(3,4-difluorophenyl)methylcarbamoyl]-2-thienyl]-2-hydroxy-6-isobutyl-pyridine-3-carboxylate

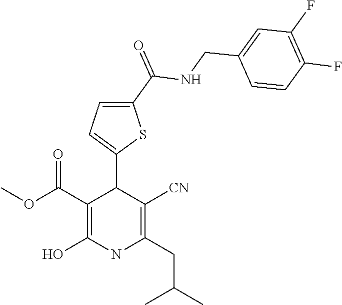 [SMILES: COC(=O)C1=C(O)NC(CC(C)C)=C(C#N)C1c1ccc(C(=O)NCc2ccc(F)c(F)c2)s1]

To a solution of methyl 5-cyano-4-[5-[(3,4-difluorophenyl)methylcarbamoyl]-2-thienyl]-2-hydroxy-6-isobutyl-1,4-dihydropyridine-3-carboxylate (1.5 g, crude) in DCM (30 mL) was added CAN (5.06 g, 9.23 mmol). The mixture was stirred at 70° C. for 2 hrs. The reaction mixture was concentrated under reduced pressure to give a residue. The residue was purified by column chromatography (SiO2, 0-5% of DCM in MeOH) to give methyl 5-cyano-4-[5-[(3,4-difluorophenyl)methylcarbamoyl]-2-thienyl]-2-hydroxy-6-isobutyl-pyridine-3-carboxylate (300 mg). LC-MS: m/z 486.2 (M+H)⁺.

### Step C methyl 2-chloro-5-cyano-4-[5-[(3,4-difluorophenyl)methylcarbamoyl]-2-thienyl]-6-isobutyl-pyridine-3-carboxylate

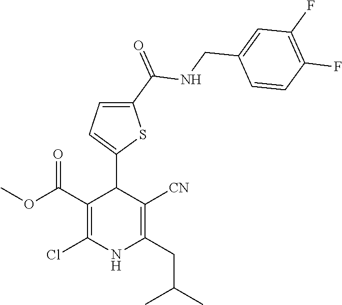 [SMILES: COC(=O)C1=C(Cl)NC(CC(C)C)=C(C#N)C1c1ccc(C(=O)NCc2ccc(F)c(F)c2)s1]

To a solution of methyl 5-cyano-4-(5-((3,4-difluorobenzyl)carbamoyl)thiophen-2-yl)-2-hydroxy-6-isobutylnicotinate (150 mg, 308.96 μmol) in MeCN (1 mL) was added POCl₃ (158.00 mg, 1.03 mmol). The mixture was stirred at 80° C. for 2 hrs. The reaction mixture was concentrated under reduced pressure to give a residue. The residue was purified by column chromatography (SiO₂, 0.22% of Ethyl acetate in Petroleum ether) to give methyl 2-chloro-5-cyano-4-[5-[(3,4-difluorophenyl)methylcarbamoyl]-2-thienyl]-6-isobutyl-pyridine-3-carboxylate (100 mg, 48.17%, 75% purity). LC-MS: m/z 504.2 (M+H)⁺.

### Step D methyl 5-cyano-4-[5-[(3,4-difluorophenyl)methylcarbamoyl]-2-thienyl]-2-[(4-fluorophenyl)methoxy]-6-isobutyl-pyridine-3-carboxylate

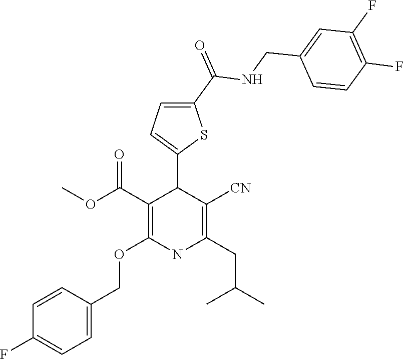 [SMILES: COC(=O)C1=C(OCc2ccc(F)cc2)NC(CC(C)C)=C(C#N)C1c1ccc(C(=O)NCc2ccc(F)c(F)c2)s1]

To a solution of (4-fluorophenyl)methanol (30.03 mg, 238.12 μmol) in DMF (2 mL) was added Cs₂CO₃ (129.31 mg, 396.87 μmol) and methyl 2-chloro-5-cyano-4-[5-[(3,4-difluorophenyl)methylcarbamoyl]-2-thienyl]-6-isobutyl-pyridine-3-carboxylate (100 mg, 198.43 μmol). The mixture was stirred at 50° C. for 8 hrs. The reaction mixture was concentrated under reduced pressure to give a residue. The residue was purified by column chromatography (SiO₂, 0-36% of Ethyl acetate in Petroleum ether) to give methyl 5-cyano-4-[5-[(3,4-difluorophenyl)methylcarbamoyl]-2-thienyl]-2-[(4-fluorophenyl)methoxy]-6-isobutyl-pyridine-3-carboxylate (60 mg, 50.94%).

¹H NMR (400 MHz, CDCl₃) δ 7.51 (d, J=3.91 Hz, 1H), 7.35-7.43 (m, 2H), 7.24-7.27 (m, 1H), 7.12-7.22 (m, 2H), 7.02-7.12 (m, 3H), 6.50-6.61 (m, 1H), 5.48 (s, 2H), 4.57 (d, J=5.99 Hz, 2H), 3.75 (s, 2H), 2.88 (d, J=7.21 Hz, 2H), 2.25 (q, J=6.81, 13.61 Hz, 1H), 0.99 (d, J=6.72 Hz, 6H). LC-MS: m/z 594.5 (M+H)⁺.

### Step E 5-cyano-4-[5-[(3,4-difluorophenyl)methylcarbamoyl]-2-thienyl]-2-[(4-fluorophenyl)methoxy]-6-isobutyl-pyridine-3-carboxylic acid

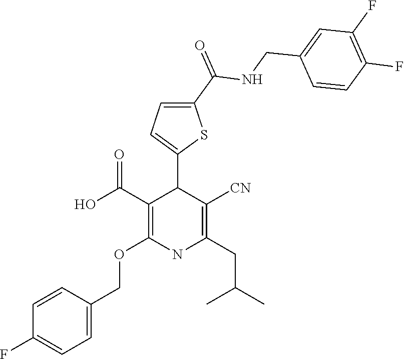 [SMILES: CC(C)CC1=C(C#N)C(c2ccc(C(=O)NCc3ccc(F)c(F)c3)s2)C(C(=O)O)=C(OCc2ccc(F)cc2)N1]

To a solution of methyl 5-cyano-4-[5-[(3,4-difluorophenyl)methylcarbamoyl]-2-thienyl]-2-[(4-fluorophenyl)methoxy]-6-isobutyl-pyridine-3-carboxylate (60 mg, 101.08 μmol) in THF (2 mL) and H₂O (1 mL) was added LiOH·H₂O (8.48 mg, 202.15 μmol). The mixture was stirred at 50° C. for 8 hrs. The reaction mixture was adjusted pH=7 with 1M HCl, and then diluted with H₂O (3 mL) and extracted with EtOAc (3 mL×2). The combined organic layers were washed with brine (3 mL×2), dried over Na₂SO₄, filtered and concentrated under reduced pressure to give a residue. The residue was purified by column chromatography (SiO₂, 0-9% of MeOH in DCM) to give 5-cyano-4-[5-[(3,4-difluorophenyl)methylcarbamoyl]-2-thienyl]-2-[(4-fluorophenyl)methoxy]-6-isobutyl-pyridine-3-carboxylic acid (60 mg, crude). LC-MS: m/z 580.1 (M+H)⁺.

### Step F 5-[3-(acetamidocarbamoyl)-5-cyano-2-[(4-fluorophenyl)methoxy]-6-isobutyl-4-pyridyl]-N-[(3,4-difluorophenyl)methyl]thiophene-2-carboxamide

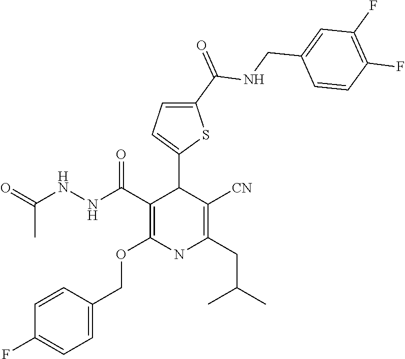 [SMILES: CC(=O)NNC(=O)C1=C(OCc2ccc(F)cc2)NC(CC(C)C)=C(C#N)C1c1ccc(C(=O)NCc2ccc(F)c(F)c2)s1]

To a solution of 5-cyano-4-[5-[(3,4-difluorophenyl)methylcarbamoyl]-2-thienyl]-2-[(4-fluorophenyl)methoxy]-6-isobutyl-pyridine-3-carboxylic acid (60 mg, 103.52 μmol) in DMF (2 mL) was added HATU (47.23 mg, 124.23 μmol) and DIEA (40.14 mg, 310.57 μmol). The mixture was stirred at 25° C. for 30 min. Then acetohydrazide (11.50 mg, 155.28 μmol) was added to the mixture and the mixture was stirred at 50° C. for 1.5 hrs. The reaction mixture was diluted with H₂O (10 mL) and extracted with EtOAc (10 mL×2). The combined organic layers were washed with brine (10 mL×2), dried over Na₂SO₄, filtered and concentrated under reduced pressure to give crude product 5-[3-(acetamidocarbamoyl)-5-cyano-2-[(4-fluorophenyl)methoxy]-6-isobutyl-4-pyridyl]-N-[(3,4-difluorophenyl)methyl]thiophene-2-carboxamide (70 mg, 82.76% yield, 77.8% purity). LC-MS: m/z 636.1 (M+H)⁺.

### Step G 5-(3-cyano-6-((4-fluorobenzyl)oxy)-2-isobutyl-5-(5-methyl-1,3,4-oxadiazol-2-yl)pyridin-4-yl)-N-(3,4-difluorobenzyl)thiophene-2-carboxamide

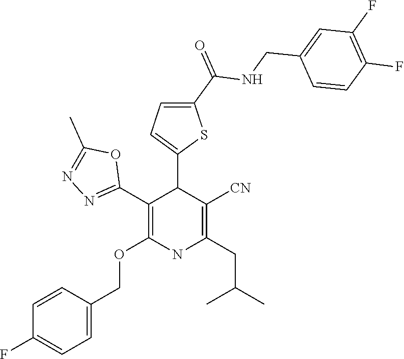 [SMILES: Cc1nnc(C2=C(OCc3ccc(F)cc3)NC(CC(C)C)=C(C#N)C2c2ccc(C(=O)NCc3ccc(F)c(F)c3)s2)o1]

To a solution of 5-[3-(acetamidocarbamoyl)-5-cyano-2-[(4-fluorophenyl)methoxy]-6-isobutyl-4-pyridyl]-N-[(3,4-difluorophenyl)methyl]thiophene-2-carboxamide (70 mg, 110.12 μmol) in DCM (3 mL) was added TEA (33.43 mg, 330.37 μmol) and 4-methylbenzenesulfonyl chloride (25.19 mg, 132.15 μmol). The mixture was stirred at 50° C. for 2 hrs. The reaction mixture was concentrated under reduced pressure to give a residue. The residue was purified by column chromatography (SiO₂, 0-100% of Ethyl acetate in Petroleum ether) to give 5-(3-cyano-6-((4-fluorobenzyl)oxy)-2-isobutyl-5-(5-methyl-1,3,4-oxadiazol-2-yl)pyridin-4-yl)-N-(3,4-difluorobenzyl)thiophene-2-carboxamide (60 mg, 74.10% yield, 84% purity). LC-MS: m/z 618.4 (M+H)⁺

### Step H 4-[5-[(3,4-difluorophenyl)methylcarbamoyl]-2-thienyl]-6-[(4-fluorophenyl)methoxy]-2-isobutyl-5-(5-methyl-1,3,4-oxadiazol-2-yl)pyridine-3-carboxamide (Compound 146)

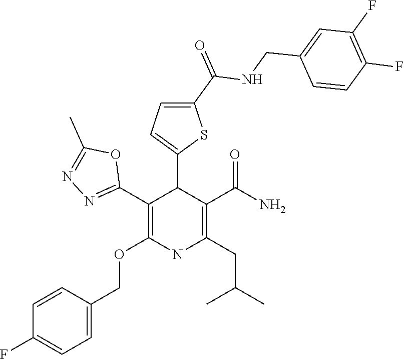 [SMILES: Cc1nnc(C2=C(OCc3ccc(F)cc3)NC(CC(C)C)=C(C(N)=O)C2c2ccc(C(=O)NCc3ccc(F)c(F)c3)s2)o1]

A mixture of 5-[5-cyano-2-[(4-fluorophenyl)methoxy]-6-isobutyl-3-(5-methyl-1,3,4-oxadiazol-2-yl)-4-pyridyl]-N-[(3,4-difluorophenyl)methyl]thiophene-2-carboxamide (60 mg, 97.14 μmol), H₂O₂ (50 mg, 30% purity) and K₂CO₃ (6.71 mg, 48.57 μmol) in DMSO (2 mL) was degassed and purged with N₂ for 3 times, and then the mixture was stirred at 25° C. for 16 hr under N₂ atmosphere. LCMS showed the reaction was not complete. H₂O₂ (210 mg, 30% purity) was added. The mixture was stirred at 25° C. for 16 hrs. The residue was diluted with water (30 mL) and extracted with EA (30 mL×2). The combined organic layers were washed with brine (30 mL), dried over Na₂SO₄, filtered and concentrated under reduced pressure to give a residue. The residue was purified by prep-HPLC (Column: Kromasil 100-5-C18; Eluent: 45% to 85% water (0.1% FA)-ACN) to give 4-[5-[(3,4-difluorophenyl)methylcarbamoyl]-2-thienyl]-6-[(4-fluorophenyl)methoxy]-2-isobutyl-5-(5-methyl-1,3,4-oxadiazol-2-yl)pyridine-3-carboxamide (2.32 mg, 3.64% yield, 96.864% purity).

¹H NMR (400 MHz, CDCl₃) δ 7.31-7.36 (m, 3H), 7.09-7.18 (m, 2H), 7.00-7.08 (m, 4H), 6.63 (br t, J=5.81 Hz, 1H), 5.49-5.68 (m, 2H), 5.44 (s, 2H), 4.49 (d, J=5.99 Hz, 2H), 2.75 (d, J=7.21 Hz, 2H), 2.50 (s, 3H), 2.32 (td, J=6.72, 13.45 Hz, 1H), 0.96 (d, J=6.60 Hz, 6H). LC-MS: m/z 636.1 (M+H)⁺.

### N-(3,4-difluorophenyl)methyl-5-{5-carbamoyl-2-[2-(p-fluorophenyl)ethyl]-6-isobutyl-3-(5-isopropyl-1,3,4-oxadiazol-2-yl)-4-pyridyl}-2-thenamide (Compound 147)

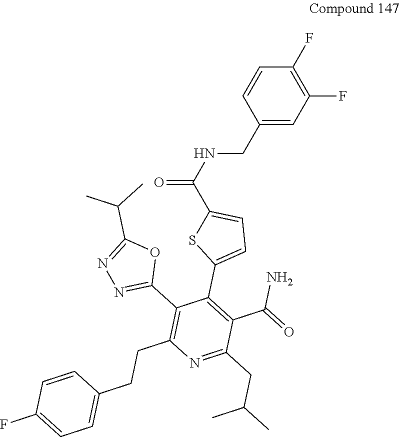 [SMILES: CC(C)Cc1nc(CCc2ccc(F)cc2)c(-c2nnc(C(C)C)o2)c(-c2ccc(C(=O)NCc3ccc(F)c(F)c3)s2)c1C(N)=O]

Compound 147 was synthesized using a similar procedure described in the Example 8 above by using the appropriate materials.

¹H NMR (400 MHz, DMSO-d₆) δ 9.11-9.17 (m, 1H), 7.88 (br s, 1H), 7.64-7.68 (m, 1H), 7.59 (br s, 1H), 7.35-7.44 (m, 1H), 7.24-7.32 (m, 1H), 7.03-7.15 (m, 5H), 6.97 (d, J=3.6 Hz, 1H), 4.39 (d, J=6 Hz, 2H), 2.92-3.11 (m, 5H), 2.73 (d, J=7.2 Hz, 2H), 2.25-2.35 (m, 1H), 1.05 (d, J=6.8 Hz, 6H), 0.93 (d, J=6.8 Hz, 6H). ¹⁹F NMR (377 MHz, DMSO-d₆) δ −117.27, −138.94, −141.41. LC-MS: m/z 662.2 (M+H)⁺.

### Example 23

### N-(3,4-difluorophenyl)methyl-5-{5-carbamoyl-2-[2-(p-fluorophenyl)ethyl]-6-isobutyl-3-[5-(trifluoromethyl)-1,3,4-oxadiazol-2-yl]-4-pyridyl}-2-thenamide (Compound 148)

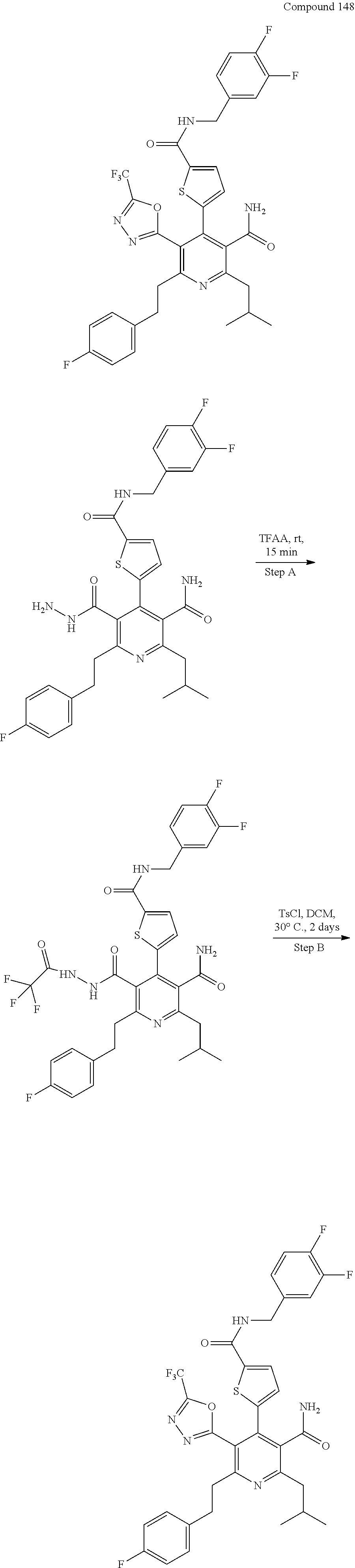 [SMILES: CC(C)Cc1nc(CCc2ccc(F)cc2)c(-c2nnc(C(F)(F)F)o2)c(-c2ccc(C(=O)NCc3ccc(F)c(F)c3)s2)c1C(N)=O ; CC(C)Cc1nc(CCc2ccc(F)cc2)c(C(=O)NN)c(-c2ccc(C(=O)NCc3ccc(F)c(F)c3)s2)c1C(N)=O ; CC(C)Cc1nc(CCc2ccc(F)cc2)c(C(=O)NNC(=O)C(F)(F)F)c(-c2ccc(C(=O)NCc3ccc(F)c(F)c3)s2)c1C(N)=O ; CC(C)Cc1nc(CCc2ccc(F)cc2)c(-c2nnc(C(F)(F)F)o2)c(-c2ccc(C(=O)NCc3ccc(F)c(F)c3)s2)c1C(N)=O]

### Step A 4-(5-((3,4-difluorobenzyl)carbamoyl)thiophen-2-yl)-6-(4-fluorophenethyl)-2-isobutyl-5-(2-(2,2,2-trifluoroacetyl)hydrazine-1-carbonyl)nicotinamide

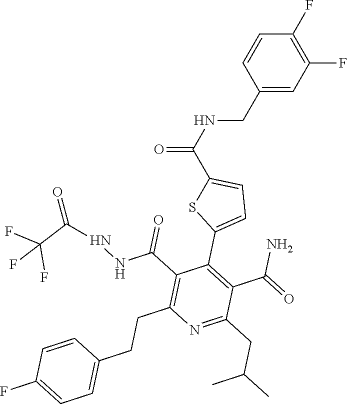 [SMILES: CC(C)Cc1nc(CCc2ccc(F)cc2)c(C(=O)NNC(=O)C(F)(F)F)c(-c2ccc(C(=O)NCc3ccc(F)c(F)c3)s2)c1C(N)=O]

A mixture of 4-(5-((3,4-difluorobenzyl)carbamoyl)thiophen-2-yl)-6-(4-fluorophenethyl)-5-(hydrazinecarbonyl)-2-isobutylnicotinamide (70 mg, 0.115 mmol) in TFAA (1 mL) was stirred at room temperature for 15 min. The reaction mixture was adjusted to pH=8 with aq. NaHCO₃ solution, extracted with EtOAc (15 mL×3). The organic layers were combined, dried over Na₂SO₄, filtered and concentrated under vacuum to dryness. The residue was purified by Prep-TLC (EtOAc/PE=9/1) to give 4-(5-((3,4-difluorobenzyl)carbamoyl)thiophen-2-yl)-6-(4-fluorophenethyl)-2-isobutyl-5-(2-(2,2,2-trifluoroacetyl)hydrazine-1-carbonyl)nicotinamide (20 mg, 24.68%). LC-MS: m/z 706.1 (M+H)⁺.

### Step B N-(3,4-difluorophenyl)methyl-5-{5-carbamoyl-2-[2-(p-fluorophenyl)ethyl]-6-isobutyl-3-[5-(trifluoromethyl)-1,3,4-oxadiazol-2-yl]-4-pyridyl}-2-thenamide (Compound 148)

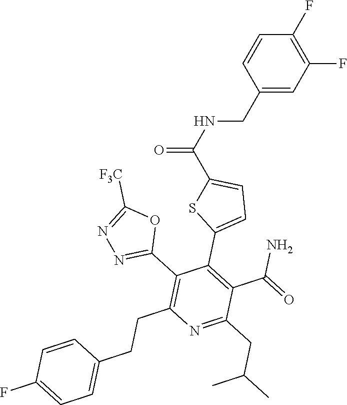 [SMILES: CC(C)Cc1nc(CCc2ccc(F)cc2)c(-c2nnc(C(F)(F)F)o2)c(-c2ccc(C(=O)NCc3ccc(F)c(F)c3)s2)c1C(N)=O]

To a solution of 4-(5-((3,4-difluorobenzyl)carbamoyl)thiophen-2-yl)-6-(4-fluorophenethyl)-2-isobutyl-5-(2-(2,2,2-trifluoroacetyl)hydrazine-1-carbonyl)nicotinamide (20 mg, 0.028 mmol) in DCM (3 mL) were added TEA (0.004 mL, 0.028 mmol), Tosyl chloride (6.41 mg, 0.034 mmol) and the reaction mixture was stirred at 30° C. for over the weekend. The reaction mixture was diluted with water (30 mL), extracted with EtOAc (15 mL*3). The organic layers were combined, dried over Na₂SO₄, filtered and concentrated under vacuum to dryness. The residue was purified by Prep-HPLC to N-(3,4-difluorophenyl)methyl-5-{5-carbamoyl-2-[2-(p-fluorophenyl)ethyl]-6-isobutyl-3-[5-(trifluoromethyl)-1,3,4-oxadiazol-2-yl]-4-pyridyl}-2-thenamide (2.03 mg, 10.42%). ¹H NMR (400 MHz, DMSO-d₆) δ 9.16 (t, J=5.6 Hz, 1H), 7.92 (s, 1H), 7.62-7.70 (m, 2H), 7.35-7.44 (m, 1H), 7.25-7.33 (m, 1H), 7.10-7.18 (m, 3H), 6.95-7.06 (m, 3H), 4.41 (d, J=6.0 Hz, 2H), 3.16-3.23 (m, 2H), 3.00-3.08 (m, 2H), 2.77 (d, J=6.8 Hz, 2H), 2.28-2.38 (m, 1H), 0.96 (d, J=6.8 Hz, 6H). ¹⁹F NMR (377 MHz, DMSO-d₆) δ −64.84, —117.22, −139.01, −141.38. LC-MS: m/z 688.1 (M+H)⁺.

### Example 24

### N-(3,4-difluorophenyl)methyl-5-{5-carbamoyl-2-[2-(p-fluorophenyl)ethyl]-6-isobutyl-3-(5-methyl-1,3,4-thiadiazol-2-yl)-4-pyridyl}-2-thenamide (Compound 149)

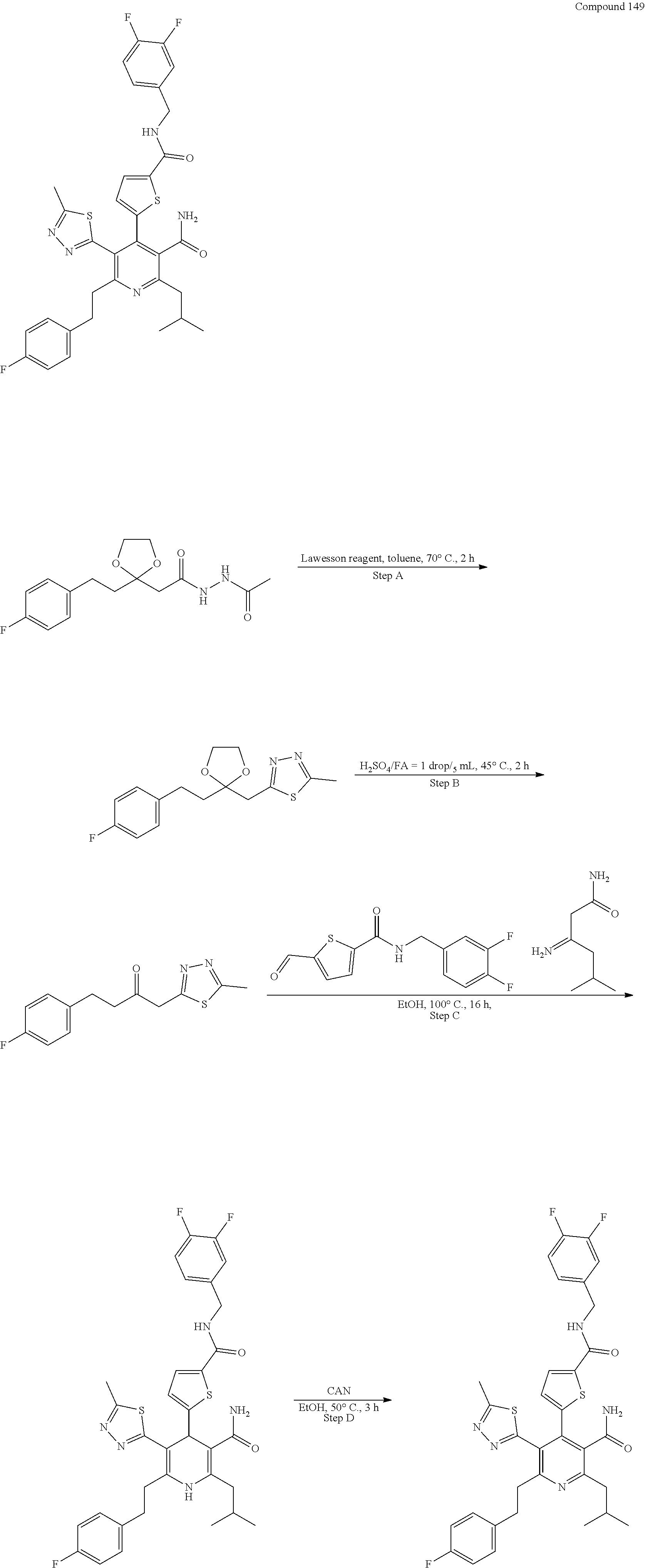

### Step A 2-((2-(4-fluorophenethyl)-1,3-dioxolan-2-yl)methyl)-5-methyl-1,3,4-thiadiazole

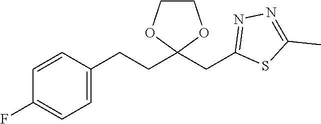 [SMILES: Cc1nnc(CC2(CCc3ccc(F)cc3)OCCO2)s1]

To a solution of Lawesson reagent (208.53 mg, 0.516 mmol) in toluene (8 mL) were added N′-acetyl-2-(2-(4-fluorophenethyl)-1,3-dioxolan-2-yl)acetohydrazide (200 mg, 0.644 mmol), and the reaction mixture was stirred at 70° C. for 2 hr under N₂. After the reaction was completed, the mixture was concentrated under vacuum to dryness. The residue was purified by column chromatography on silica gel (DCM/MeOH=20/1) to give 2-((2-(4-fluorophenethyl)-1,3-dioxolan-2-yl)methyl)-5-methyl-1,3,4-thiadiazole (129 mg, 64.91%). LC-MS: m/z 308.9 (M+H)⁺.

### Step B 4-(4-fluorophenyl)-1-(5-methyl-1,3,4-thiadiazol-2-yl)butan-2-one

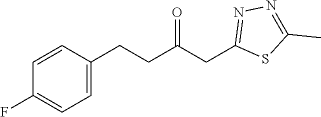 [SMILES: Cc1nnc(CC(=O)CCc2ccc(F)cc2)s1]

A mixture of 2-((2-(4-fluorophenethyl)-1,3-dioxolan-2-yl)methyl)-5-methyl-1,3,4-thiadiazole (129 mg, 0.42 mmol) in con. H₂SO₄/FA (1 drop/5 mL) was stirred at 45° C. for 2 hours. The mixture was quenched with H₂O (30 mL), extracted with EtOAc (30 mL*2). The combined organic layers were washed with brine (50 mL), dried over anhydrous Na₂SO₄, filtered and concentrated. The residue was purified by column (PE/EA=1/1) to give 4-(4-fluorophenyl)-1-(5-methyl-1,3,4-thiadiazol-2-yl)butan-2-one (101 mg, 91.8%). LC-MS: m/z 264.8 (M+H)⁺.

### Step C 4-(5-((3,4-difluorobenzyl)carbamoyl)thiophen-2-yl)-6-(4-fluorophenethyl)-2-isobutyl-5-(5-methyl-1,3,4-thiadiazol-2-yl)-1,4-dihydropyridine-3-carboxamide

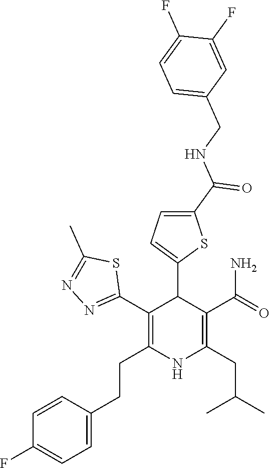 [SMILES: Cc1nnc(C2=C(CCc3ccc(F)cc3)NC(CC(C)C)=C(C(N)=O)C2c2ccc(C(=O)NCc3ccc(F)c(F)c3)s2)s1]

A mixture of 4-(4-fluorophenyl)-1-(5-methyl-1,3,4-thiadiazol-2-yl)butan-2-one (50 mg, 0.19 mmol), N-(3,4-difluorobenzyl)-5-formylthiophene-2-carboxamide (53 mg, 0.19 mmol) and 3-imino-5-methylhexanamide (27 mg, 0.19 mmol) in EtOH (2 mL) was stirred at 100° C. for 16 hours in seal tube. The mixture was concentrated under reduced pressure to give crude 4-(5-((3,4-difluorobenzyl)carbamoyl)thiophen-2-yl)-6-(4-fluorophenethyl)-2-isobutyl-5-(5-methyl-1,3,4-thiadiazol-2-yl)-1,4-dihydropyridine-3-carboxamide (80 mg, crude). LC-MS: m/z 652.2 (M+H)⁺.

### Step D N-(3,4-difluorophenyl)methyl-5-{5-carbamoyl-2-[2-(p-fluorophenyl)ethyl]-6-isobutyl-3-(5-methyl-1,3,4-thiadiazol-2-yl)-4-pyridyl}-2-thenamide (Compound 149)

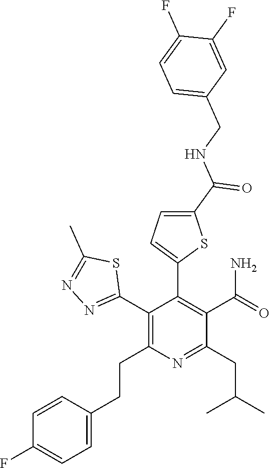 [SMILES: Cc1nnc(-c2c(CCc3ccc(F)cc3)nc(CC(C)C)c(C(N)=O)c2-c2ccc(C(=O)NCc3ccc(F)c(F)c3)s2)s1]

A mixture of 4-(5-((3,4-difluorobenzyl)carbamoyl)thiophen-2-yl)-6-(4-fluorophenethyl)-2-isobutyl-5-(5-methyl-1,3,4-thiadiazol-2-yl)-1,4-dihydropyridine-3-carboxamide (80 mg, crude), diammonium cerium(IV) nitrate (101 mg, 0.18 mmol) in EtOH (2 mL) was stirred at 50° C. for 3 hours. The solution was filtered and the filtrate was purified by Prep-HPLC (0.1% NH₃H₂O/H₂O/CH₃CN) to give N-(3,4-difluorophenyl)methyl-5-{5-carbamoyl-2-[2-(p-fluorophenyl)ethyl]-6-isobutyl-3-(5-methyl-1,3,4-thiadiazol-2-yl)-4-pyridyl}-2-thenamide (30.44 mg, 37.5%).

¹H NMR (400 MHz, DMSO-d₆) δ 9.08 (t, J=5.6 Hz, 1H), 7.83 (br s, 1H), 7.60 (d, J=4.0 Hz, 1H), 7.52 (br s, 1H), 7.30-7.43 (m, 2H), 7.01-7.18 (m, 5H), 6.93 (d, J=3.6 Hz, 1H), 4.38 (d, J=6.0 Hz, 2H), 2.83-2.99 (m, 4H), 2.72 (d, J=7.2 Hz, 2H), 2.68 (s, 3H), 2.24-2.35 (m, 1H), 0.94 (d, J=6.8 Hz, 6H). ¹⁹F NMR (377 MHz, DMSO-d₆) δ −117.31, −138.83, −141.31. LC-MS: m/z 650.2 (M+H)⁺.

### Example 25

### N-(3,4-difluorophenyl)methyl-5-{5-carbamoyl-2-[2-(p-fluorophenyl)ethyl]-6-isobutyl-3-(5-methyl-1,3-oxazol-2-yl)-4-pyridyl}-2-thenamide (Compound 150)

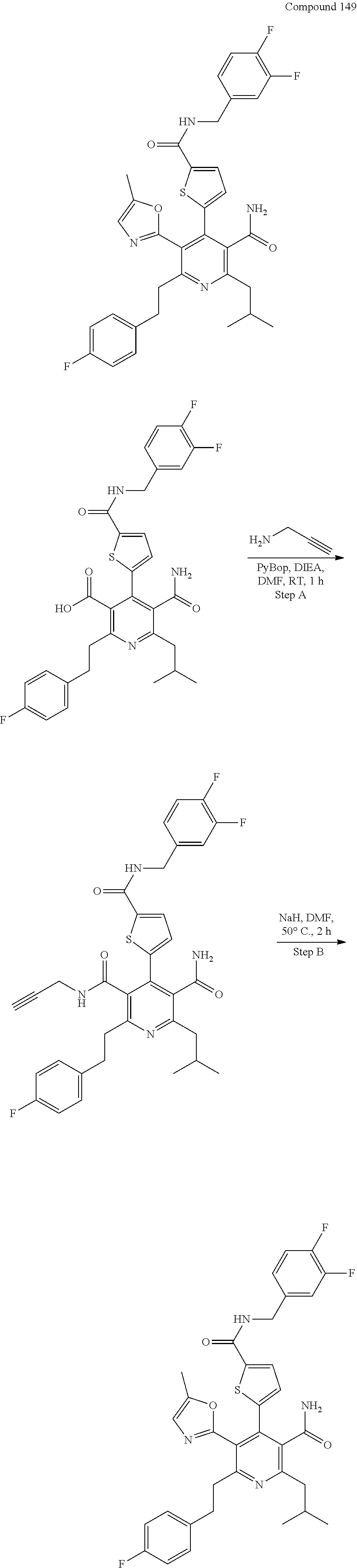

### Step A 4-(5-((3,4-difluorobenzyl)carbamoyl)thiophen-2-yl)-2-(4-fluorophenethyl)-6-isobutyl-N-(prop-2-yn-1-yl)pyridine-3,5-dicarboxamide

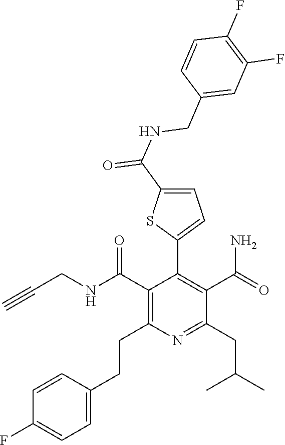 [SMILES: C#CCNC(=O)c1c(CCc2ccc(F)cc2)nc(CC(C)C)c(C(N)=O)c1-c1ccc(C(=O)NCc2ccc(F)c(F)c2)s1]

To a solution of 5-carbamoyl-4-(5-((3,4-difluorobenzyl)carbamoyl)thiophen-2-yl)-2-(4-fluorophenethyl)-6-isobutylnicotinic acid (100 mg, 0.168 mmol) in DMF (2 mL) were added aminoacetonitrile (14.12 mg, 0.252 mmol), DIEA (65.10 mg, 0.504 mmol), and PyBOP (131.06 mg, 0.252 mmol), and the reaction mixture was stirred at room temperature for 1 hr. After the reaction was completed, the mixture was washed with water (30 mL), extracted with EtOAc (15 mL×3). The organic layers were combined, dried over Na₂SO₄, filtered and concentrated under vacuum to dryness. The residue was purified by column chromatography on silica gel (EtOAc=100%) to give 4-(5-((3,4-difluorobenzyl)carbamoyl)thiophen-2-yl)-2-(4-fluorophenethyl)-6-isobutyl-N-(prop-2-yn-1-yl)pyridine-3,5-dicarboxamide (103 mg, 96.81%). LC-MS: m/z 633.3 (M+H)⁺.

### Step B N-(3,4-difluorophenyl)methyl-5-{5-carbamoyl-2-[2-(p-fluorophenyl)ethyl]-6-isobutyl-3-(5-methyl-1,3-oxazol-2-yl)-4-pyridyl}-2-thenamide (Compound 150)

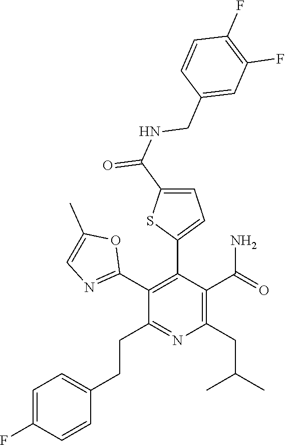 [SMILES: Cc1cnc(-c2c(CCc3ccc(F)cc3)nc(CC(C)C)c(C(N)=O)c2-c2ccc(C(=O)NCc3ccc(F)c(F)c3)s2)o1]

To a solution of 4-(5-((3,4-difluorobenzyl)carbamoyl)thiophen-2-yl)-2-(4-fluorophenethyl)-6-isobutyl-N-(prop-2-yn-1-yl)pyridine-3,5-dicarboxamide (45 mg, 0.074 mmol) in DMF (1 mL) was added NaH (60%, 8.52 mg, 0.355 mmol), and the reaction mixture was stirred at 50° C. for 2 hrs. After the reaction was completed, the mixture was washed with water (30 mL), extracted with EtOAc (10 mL*3). The organic layers were combined, dried over Na₂SO₄, filtered and concentrated under vacuum to dryness. The residue was purified by Prep-HPLC to give N-(3,4-difluorophenyl)methyl-5-{5-carbamoyl-2-[2-(p-fluorophenyl)ethyl]-6-isobutyl-3-(5-methyl-1,3-oxazol-2-yl)-4-pyridyl}-2-thenamide (1.58 mg, yield: 3.51%).

¹H NMR (400 MHz, DMSO-d₆) δ 9.09 (t, J=6.0 Hz, 1H), 7.86 (s, 1H), 7.63 (d, J=3.6 Hz, 1H), 7.54 (s, 1H), 7.29-7.45 (m, 2H), 7.01-7.11 (m, 5H), 6.97 (d, J=4.0 Hz, 1H), 6.94 (s, 1H), 4.40 (d, J=5.6 Hz, 2H), 2.86-3.00 (m, 4H), 2.71 (d, J=6.8 Hz, 2H), 2.22-2.32 (m, 1H), 2.17 (s, 3H), 0.93 (d, J=6.4 Hz, 6H). ¹⁹F NMR (377 MHz, DMSO-d₆) δ −117.37, −138.87, −141.36. LC-MS: m/z 633.1 (M+H)⁺.

### Example 26

### N-(3,4-difluorophenyl)methyl-5-{5-carbamoyl-2-[2-(p-fluorophenyl)ethyl]-6-isobutyl-3-(3-methyl-5-isoxazolyl)-4-pyridyl}-2-thenamide (Compound 151) and N-(3,4-difluorophenyl)methyl-5-{5-carbamoyl-2-[2-(p-fluorophenyl)ethyl]-6-isobutyl-3-(2-methyl-1,3-oxazol-5-yl)-4-pyridyl}-2-thenamide (Compound 152)

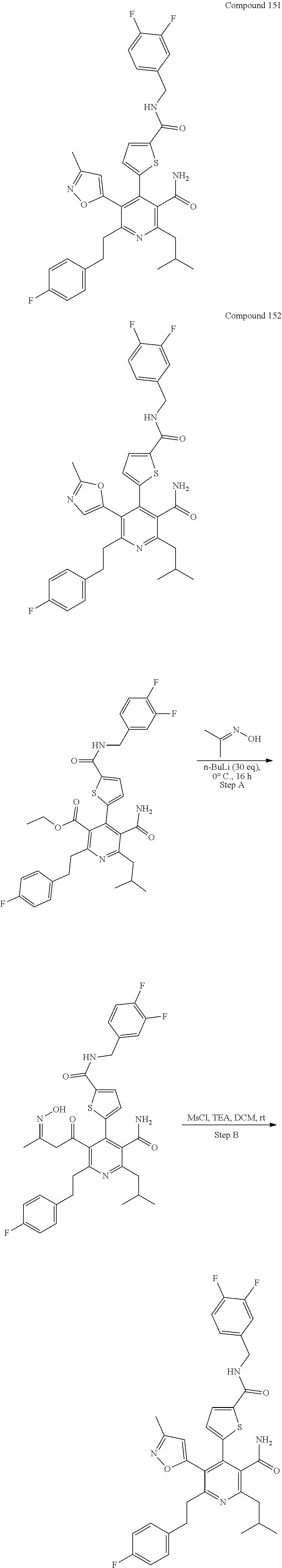

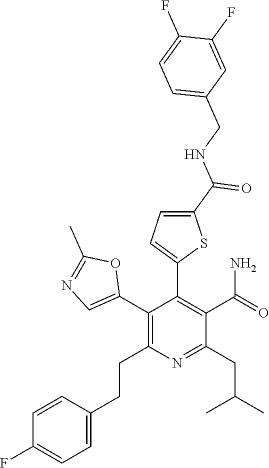 [SMILES: Cc1ncc(-c2c(CCc3ccc(F)cc3)nc(CC(C)C)c(C(N)=O)c2-c2ccc(C(=O)NCc3ccc(F)c(F)c3)s2)o1]

### Step A 4-(5-((3,4-difluorobenzyl)carbamoyl)thiophen-2-yl)-2-(4-fluorophenethyl)-6-isobutyl-N-(prop-2-yn-1-yl)pyridine-3,5-dicarboxamide

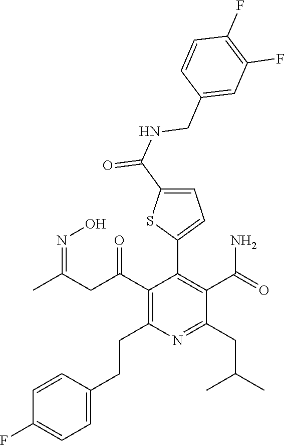 [SMILES: C/C(CC(=O)c1c(CCc2ccc(F)cc2)nc(CC(C)C)c(C(N)=O)c1-c1ccc(C(=O)NCc2ccc(F)c(F)c2)s1)=N/O]

To a solution of propan-2-one oxime (899 mg, 12.3 mmol) in THF (20 mL) was added n-BuLi (2.5 M, 7.4 mL) dropwise at 0° C., under Ar. After 1 hr, ethyl 5-carbamoyl-4-(5-((3,4-difluorobenzyl)carbamoyl)thiophen-2-yl)-2-(4-fluorophenethyl)-6-isobutylnicotinate (384 mg, 0.616 mmol) in THF (5 mL) was added into the mixture. The reaction mixture was stirred at room temperature for 16 hrs. The reaction mixture was diluted with EtOAc (20 mL) and quenched with water (5 mL). Then, the mixture was extracted with EtOAc (10 mL*3). The organic layer was separated, washed with brine (10 mL), and concentrated in vacuo. The residue was purified by silica gel column chromatography, eluting with 15% EtOAc in petroleum ether to give (Z)-4-(5-((3,4-difluorobenzyl)carbamoyl)thiophen-2-yl)-6-(4-fluorophenethyl)-5-(3-(hydroxyimino)butanoyl)-2-isobutylnicotinamide (150 mg, 37.5%). LC-MS: m/z 651.2 (M+H)⁺.

### Step B N-(3,4-difluorophenyl)methyl-5-{5-carbamoyl-2-[2-(p-fluorophenyl)ethyl]-6-isobutyl-3-(3-methyl-5-isoxazolyl)-4-pyridyl}-2-thenamide (Compound 151) and N-(3,4-difluorophenyl)methyl-5-{5-carbamoyl-2-[2-(p-fluorophenyl)ethyl]-6-isobutyl-3-(2-methyl-1,3-oxazol-5-yl)-4-pyridyl}-2-thenamide (Compound 152)

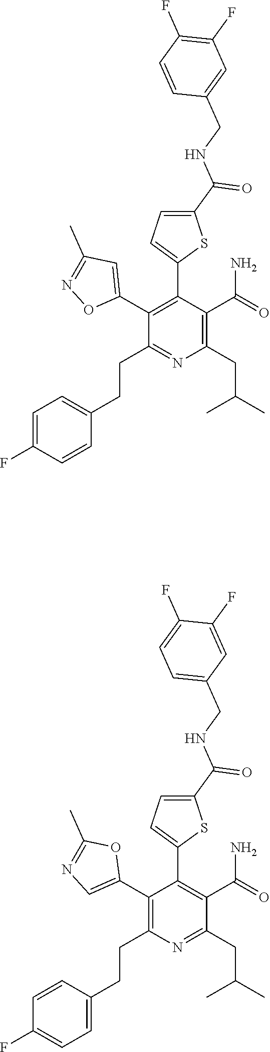 [SMILES: Cc1cc(-c2c(CCc3ccc(F)cc3)nc(CC(C)C)c(C(N)=O)c2-c2ccc(C(=O)NCc3ccc(F)c(F)c3)s2)on1 ; Cc1ncc(-c2c(CCc3ccc(F)cc3)nc(CC(C)C)c(C(N)=O)c2-c2ccc(C(=O)NCc3ccc(F)c(F)c3)s2)o1]

To a solution of (Z)-4-(5-((3,4-difluorobenzyl)carbamoyl)thiophen-2-yl)-6-(4-fluorophenethyl)-5-(3-(hydroxyimino)butanoyl)-2-isobutylnicotinamide (150 mg, 0.231 mmol) in DCM (3 mL) was added TEA (70 mg, 0.692 mmol) and MsCl (32 mg, 0.277 mmol) at 0° C., and the reaction mixture was stirred at room temperature for 2 hours. The reaction was diluted with EtOAc (15 mL) and quenched with water (5 mL). Then, the mixture was extracted with EtOAc (15 mL*3). The organic layer was separated, washed with further saturated NaCl solution (10 mL), and concentrated in vacuo. The residue was purified by prep-HPLC (0.03% FA) to give N-(3,4-difluorophenyl)methyl-5-{5-carbamoyl-2-[2-(p-fluorophenyl)ethyl]-6-isobutyl-3-(3-methyl-5-isoxazolyl)-4-pyridyl}-2-thenamide (2.99 mg, 2.05%) and N-(3,4-difluorophenyl)methyl-5-{5-carbamoyl-2-[2-(p-fluorophenyl)ethyl]-6-isobutyl-3-(2-methyl-1,3-oxazol-5-yl)-4-pyridyl}-2-thenamide (3.57 mg, 2.45%).

Compound 151: ¹H NMR (400 MHz, DMSO-d₆) δ 9.05-9.16 (m, 1H), 7.84 (br s, 1H), 7.59-7.67 (m, 1H), 7.52 (br s, 1H), 7.29-7.42 (m, 2H), 7.04-7.16 (m, 5H), 6.92-7.00 (m, 1H), 6.25 (s, 1H), 4.32-4.36 (m, 2H), 2.78-2.95 (m, 4H), 2.65-2.73 (m, 2H), 2.25-2.33 (m, 1H), 2.18 (s, 3H), 0.93 (d, J=6.0 Hz, 6H). ¹⁹F NMR (377 MHz, DMSO-d₆) δ −117.29, −138.85, −141.35. LC-MS: m/z 633.2 (M+H)⁺.

Compound 152: ¹H NMR (400 MHz, DMSO-d₆) δ 9.22 (t, J=5.6 Hz, 1H), 7.84 (br s, 1H), 7.81 (d, J=3.6 Hz, 1H), 7.55 (br s, 1H), 7.34-7.44 (m, 2H), 7.04-7.24 (m, 7H), 4.44 (d, J=5.6 Hz, 2H), 2.92-3.03 (m, 4H), 2.64-2.72 (m, 2H), 2.23-2.29 (m, 1H), 2.19 (s, 3H), 0.86-0.96 (m, 6H). ¹⁹F NMR (377 MHz, DMSO-d₆) δ −117.27, −138.831, −141.33. LC-MS: m/z 633.2 (M+H)⁺.

### Example 27

### N-(3,4-difluorophenyl)methyl-5-{5-carbamoyl-2-[2-(p-fluorophenyl)ethyl]-6-isobutyl-3-(4-methyl-1,3-oxazol-5-yl)-4-pyridyl}-2-thenamide (Compound 153)

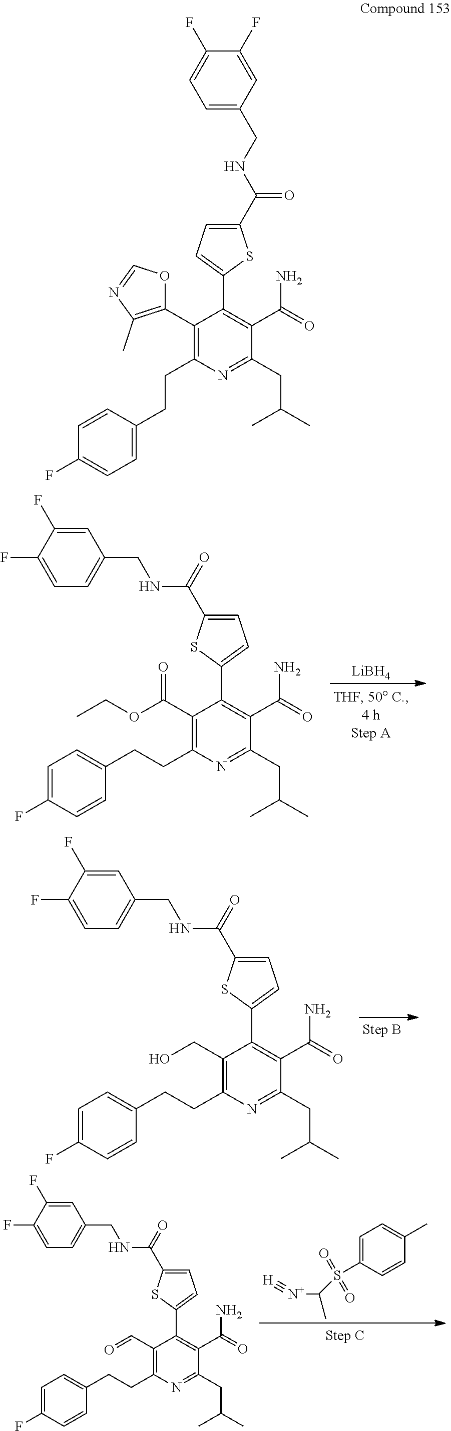

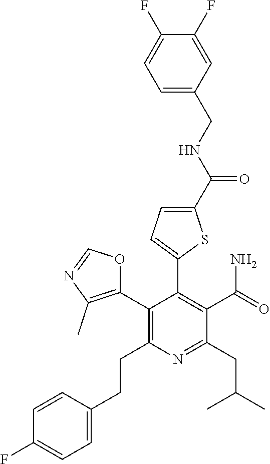 [SMILES: Cc1ncoc1-c1c(CCc2ccc(F)cc2)nc(CC(C)C)c(C(N)=O)c1-c1ccc(C(=O)NCc2ccc(F)c(F)c2)s1]

### Step A 4-(5-((3,4-difluorobenzyl)carbamoyl)thiophen-2-yl)-6-(4-fluorophenethyl)-5-(hydroxymethyl)-2-isobutylnicotinamide

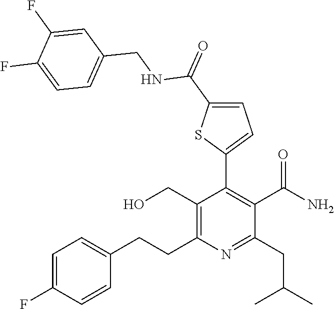 [SMILES: CC(C)Cc1nc(CCc2ccc(F)cc2)c(CO)c(-c2ccc(C(=O)NCc3ccc(F)c(F)c3)s2)c1C(N)=O]

A mixture of ethyl 5-carbamoyl-4-(5-((3,4-difluorobenzyl)carbamoyl)thiophen-2-yl)-2-(4-fluorophenethyl)-6-isobutylnicotinate (150 mg, 0.24 mmol) in THF (6 mL) under N₂ was cooled down to 0° C., then LiBH₄ (53 mg, 2.4 mmol) was added, the reaction mixture was stirred at 50° C. for 4 hrs. The reaction mixture was cooled down to room temperature, concentrated in vacuo and purified by Prep-TLC (DCM:MeOH=10:1) to give 4-(5-((3,4-difluorobenzyl)carbamoyl)thiophen-2-yl)-6-(4-fluorophenethyl)-5-(hydroxymethyl)-2-isobutylnicotinamide (50 mg, 35.7%). LC-MS: m/z 582.2 (M+H)⁺.

### Step B 4-(5-((3,4-difluorobenzyl)carbamoyl)thiophen-2-yl)-6-(4-fluorophenethyl)-5-formyl-2-isobutylnicotinamide

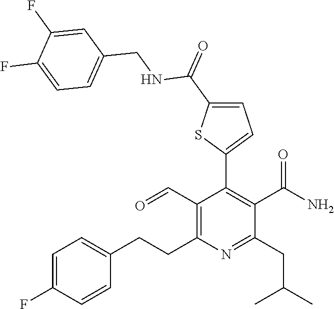 [SMILES: CC(C)Cc1nc(CCc2ccc(F)cc2)c(C=O)c(-c2ccc(C(=O)NCc3ccc(F)c(F)c3)s2)c1C(N)=O]

To a mixture of 4-(5-((3,4-difluorobenzyl)carbamoyl)thiophen-2-yl)-6-(4-fluorophenethyl)-5-(hydroxymethyl)-2-isobutylnicotinamide (50 mg, 0.086 mmol) in DMSO (2 mL) was added IBX (72 mg, 0.26 mmol), the reaction mixture was stirred at 50° C. for 3 hrs. The reaction mixture was cooled down to room temperature, concentrated in vacuo and purified by Prep-TLC (DCM:MeOH=10:1) to give 4-(5-((3,4-difluorobenzyl)carbamoyl)thiophen-2-yl)-6-(4-fluorophenethyl)-5-formyl-2-isobutylnicotinamide (25 mg, 50.17%). LC-MS: m/z 580.1 (M+H)⁺.

### Step C N-(3,4-difluorophenyl)methyl-5-{5-carbamoyl-2-[2-(p-fluorophenyl)ethyl]-6-isobutyl-3-(4-methyl-1,3-oxazol-5-yl)-4-pyridyl}-2-thenamide (Compound 153)

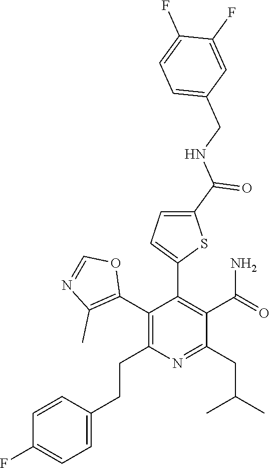 [SMILES: Cc1ncoc1-c1c(CCc2ccc(F)cc2)nc(CC(C)C)c(C(N)=O)c1-c1ccc(C(=O)NCc2ccc(F)c(F)c2)s1]

To a mixture of 4-(5-((3,4-difluorobenzyl)carbamoyl)thiophen-2-yl)-6-(4-fluorophenethyl)-5-formyl-2-isobutylnicotinamide (25 mg, 0.043 mmol) in MeOH (0.5 ml) was added 1-(toluene-4-sulfonyl)-ethyl isocyanide (11 mg, 0.052 mmol) and the reaction mixture was stirred at 70° C. for 2 hrs. The reaction mixture was cooled down to room temperature, concentrated in vacuo and purified by prep-HPLC (FA) to give N-(3,4-difluorophenyl)methyl-5-{5-carbamoyl-2-[2-(p-fluorophenyl)ethyl]-6-isobutyl-3-(4-methyl-1,3-oxazol-5-yl)-4-pyridyl}-2-thenamide (7.07 mg, 25.9%).

¹H NMR (400 MHz, DMSO-d₆) δ 9.09 (t, J=5.6 Hz, 1H), 8.33 (s, 1H), 7.83 (s, 1H), 7.62 (d, J=3.6 Hz, 1H), 7.50 (s, 1H), 7.32-7.42 (m, 2H), 7.11-7.17 (m, 1H), 7.03-7.10 (m, 4H), 6.98 (d, J=4.0 Hz, 1H), 4.39 (d, J=6.0 Hz, 2H), 2.90-2.92 (m, 2H), 2.79-2.83 (m, 2H), 2.69-2.72 (m, 2H), 2.28-2.33 (m, 1H), 1.66 (s, 3H), 0.94 (d, J=4.8 Hz, 6H). ¹⁹F NMR (377 MHz, DMSO-d₆) δ −117.37, −138.84, −141.34. LC-MS: m/z 633.2 (M+H)⁺.

### Example 28

### N-(3,4-difluorophenyl)methyl-5-{5-carbamoyl-2-[2-(p-fluorophenyl)ethyl]-6-isobutyl-3-(5-methyl-1,2,4-oxadiazol-3-yl)-4-pyridyl}-2-thenamide (Compound 154)

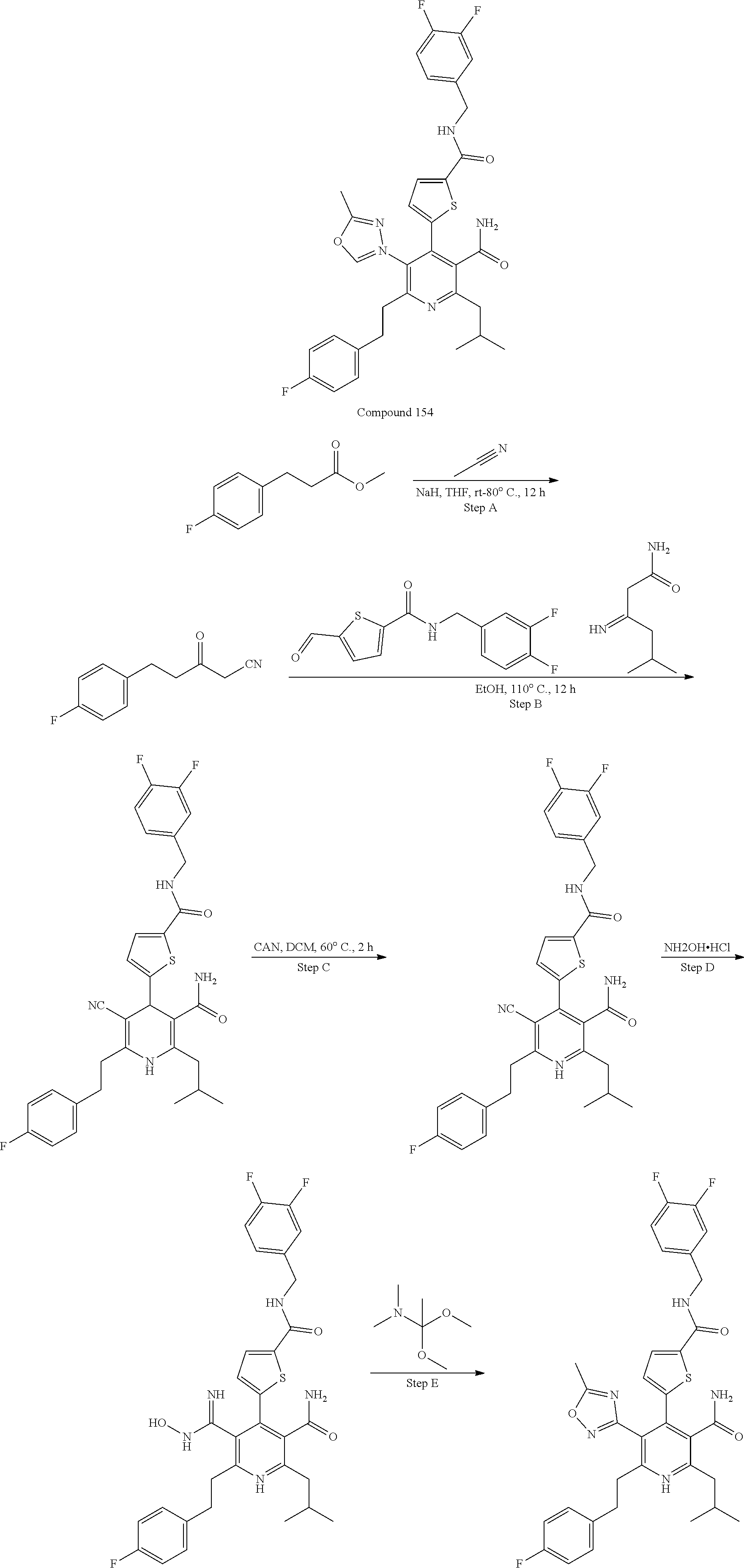

### Step A 5-(4-fluorophenyl)-3-oxopentanenitrile

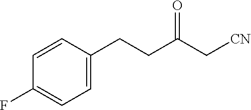 [SMILES: N#CCC(=O)CCc1ccc(F)cc1]

To a solution of acetonitrile (0.538 mL, 10.193 mmol) in THF (20 mL) was added NaH (418.88 mg, 10.19 mmol, 60% dispersion in mineral oil) at 0° C. and stirred 30 min, then methyl 3-(4-fluorophenyl)propanoate (1.0 g, 5.10 mmol) was added at 0° C., the mixture was heated to 80° C. and stirred for 12 hrs. The reaction mixture was cooled down to room temperature, quenched with H₂O (50 mL) and extracted with EtOAc (25 mL×3). The organic layer was combined and washed with brine (25 mL×2), dried over Na₂SO₄, filtered and concentrated in vacuum. The residue was purified by column chromatography (PE/EtOAc=15/1) to give 5-(4-fluorophenyl)-3-oxopentanenitrile (253 mg, yield: 25.96%).

### Step B 5-cyano-4-(5-((3,4-difluorobenzyl)carbamoyl)thiophen-2-yl)-6-(4-fluorophenethyl)-2-isobutyl-1,4-dihydropyridine-3-carboxamide

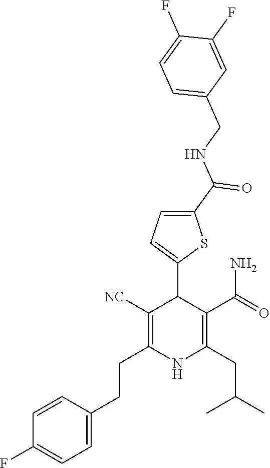 [SMILES: CC(C)CC1=C(C(N)=O)C(c2ccc(C(=O)NCc3ccc(F)c(F)c3)s2)C(C#N)=C(CCc2ccc(F)cc2)N1]

To a solution of 5-(4-fluorophenyl)-3-oxopentanenitrile (210 mg, 1.098 mmol) in EtOH (3 mL) were added 3-azanylidene-5-methylhexanamide (156.17 mg, 1.098 mmol) and N-[(3,4-difluorophenyl)methyl]-5-formylthiophene-2-carboxamide (308.92 mg, 1.098 mmol), the reaction mixture was stirred at 110° C. for overnight. The reaction mixture was cooled down to room temperature, concentrated in vacuo to give 5-cyano-4-(5-((3,4-difluorobenzyl)carbamoyl)thiophen-2-yl)-6-(4-fluorophenethyl)-2-isobutyl-1,4-dihydropyridine-3-carboxamide (650 mg, crude). MS: m/z 579.2 (M+H)⁺.

### Step C 5-cyano-4-(5-((3,4-difluorobenzyl)carbamoyl)thiophen-2-yl)-6-(4-fluorophenethyl)-2-isobutylnicotinamide

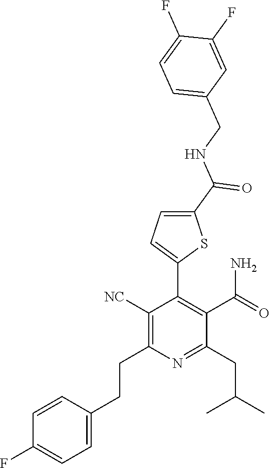 [SMILES: CC(C)Cc1nc(CCc2ccc(F)cc2)c(C#N)c(-c2ccc(C(=O)NCc3ccc(F)c(F)c3)s2)c1C(N)=O]

To a solution of ethyl 5-carbamoyl-4-(5-cyanothiophen-2-yl)-2-[2-(4-fluorophenyl)ethyl]-6-(2-methylpropyl)-1,4-dihydropyridine-3-carboxylate (650 mg, 1.12 mmol) in DCM (10 mL) was added diammonium cerium(IV) nitrate (2.433 g, 4.50 mmol), the reaction mixture was stirred at 60° C. for 2 hrs. The reaction mixture was cooled down to room temperature, quenched with H₂O (50 mL) and extracted with EtOAc (25 mL×3). The organic layer was combined and washed with brine (25 mL×2), dried over Na₂SO₄, filtered and concentrated in vacuum. The residue was purified by column chromatography (DCM/MeOH=15/1) to give 5-cyano-4-(5-((3,4-difluorobenzyl)carbamoyl)thiophen-2-yl)-6-(4-fluorophenethyl)-2-isobutylnicotinamide (230 mg, 35.51%). LC-MS: m/z 577.2 (M+H)⁺.

### Step D 4-(5-((3,4-difluorobenzyl)carbamoyl)thiophen-2-yl)-6-(4-fluorophenethyl)-5-(N-hydroxycarbamimidoyl)-2-isobutylnicotinamide

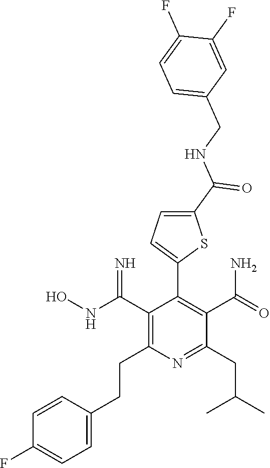 [SMILES: CC(C)Cc1nc(CCc2ccc(F)cc2)c(C(=N)NO)c(-c2ccc(C(=O)NCc3ccc(F)c(F)c3)s2)c1C(N)=O]

To a mixture of 5-cyano-4-(5-((3,4-difluorobenzyl)carbamoyl)thiophen-2-yl)-6-(4-fluorophenethyl)-2-isobutylnicotinamide (150 mg, 0.260 mmol) in EtOH (3 mL) were added NH₂OH in water (1.5 mL) and TEA (0.5 mL) at room temperature. The mixture was stirred at 120° C. for 6 hrs. The reaction mixture was cooled down to room temperature, quenched with H₂O (25 mL) and extracted with EtOAc (20 mL×3). The organic layer was combined and washed with brine (20 mL×2), dried over Na₂SO₄, filtered and concentrated in vacuum. The residual was purified by column chromatography (DCM/MeOH=20/1) to give 4-(5-((3,4-difluorobenzyl)carbamoyl)thiophen-2-yl)-6-(4-fluorophenethyl)-5-(N-hydroxycarbamimidoyl)-2-isobutylnicotinamide (70 mg, 44.14%). LC-MS: m/z 610.1 (M+H)⁺.

### Step E N-(3,4-difluorophenyl)methyl-5-{5-carbamoyl-2-[2-(p-fluorophenyl)ethyl]-6-isobutyl-3-(5-methyl-1,2,4-oxadiazol-3-yl)-4-pyridyl}-2-thenamide (Compound 154)

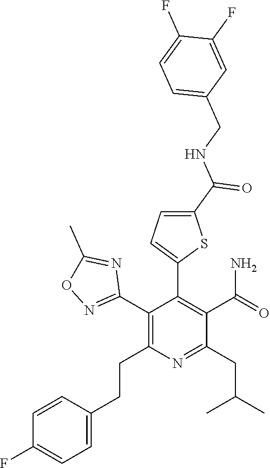 [SMILES: Cc1nc(-c2c(CCc3ccc(F)cc3)nc(CC(C)C)c(C(N)=O)c2-c2ccc(C(=O)NCc3ccc(F)c(F)c3)s2)no1]

To a mixture of 4-(5-((3,4-difluorobenzyl)carbamoyl)thiophen-2-yl)-6-(4-fluorophenethyl)-5-(N-hydroxycarbamimidoyl)-2-isobutylnicotinamide (36 mg, 0.059 mmol) in ACN (3.0 mL) was added 1,1-dimethoxy-N,N-dimethylethan-1-amine (11.80 mg, 0.089 mmol) at room temperature. The mixture was stirred at 80° C. for 3 hrs. The reaction mixture was cooled down to room temperature, concentrated in vacuum. The residue was purified by Prep-HPLC to give N-(3,4-difluorophenyl)methyl-5-{5-carbamoyl-2-[2-(p-fluorophenyl)ethyl]-6-isobutyl-3-(5-methyl-1,2,4-oxadiazol-3-yl)-4-pyridyl}-2-thenamide (9.05 mg, 24.18%).

¹H NMR (400 MHz, DMSO-d₆) δ 9.09 (t, J=6.0 Hz, 1H), 7.86 (br s, 1H), 7.61 (d, J=2.4 Hz, 1H), 7.53 (br s, 1H), 7.28-7.45 (m, 2H), 7.11-7.19 (m, 3H), 7.06 (t, J=8.0 Hz, 2H), 6.95 (d, J=2.4 Hz, 1H), 4.39 (d, J=6.0 Hz, 2H), 2.83-2.98 (m, 4H), 2.71 (d, J=6.8 Hz, 2H), 2.59 (s, 3H), 2.20-2.36 (m, 1H), 0.92 (d, J=6.8 Hz, 6H). ¹⁹F NMR (377 MHz, DMSO-d₆) δ −117.38, −138.84, −141.33. LC-MS: m/z 634.1 (M+H)⁺.

### Example 29

### 4-(5-((3,4-difluorobenzyl)carbamoyl)thiophen-2-yl)-6-(4-fluorophenethyl)-2-isopropoxy-5-(5-methyl-1,3,4-oxadiazol-2-yl)nicotinamide (Compound 167)

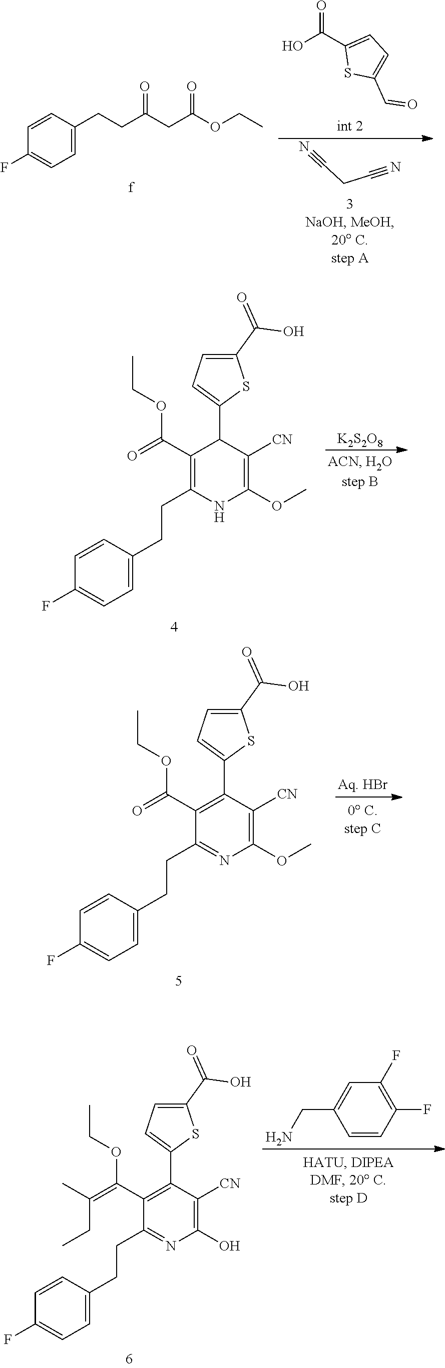

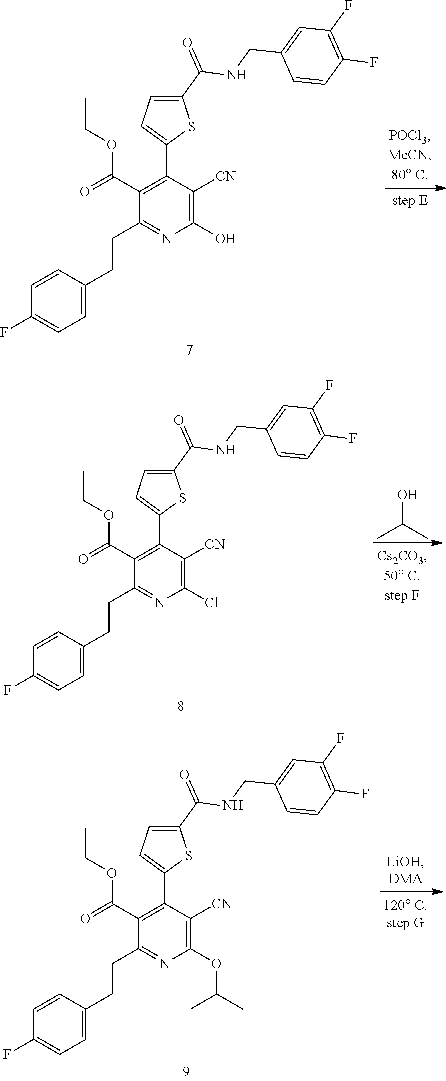

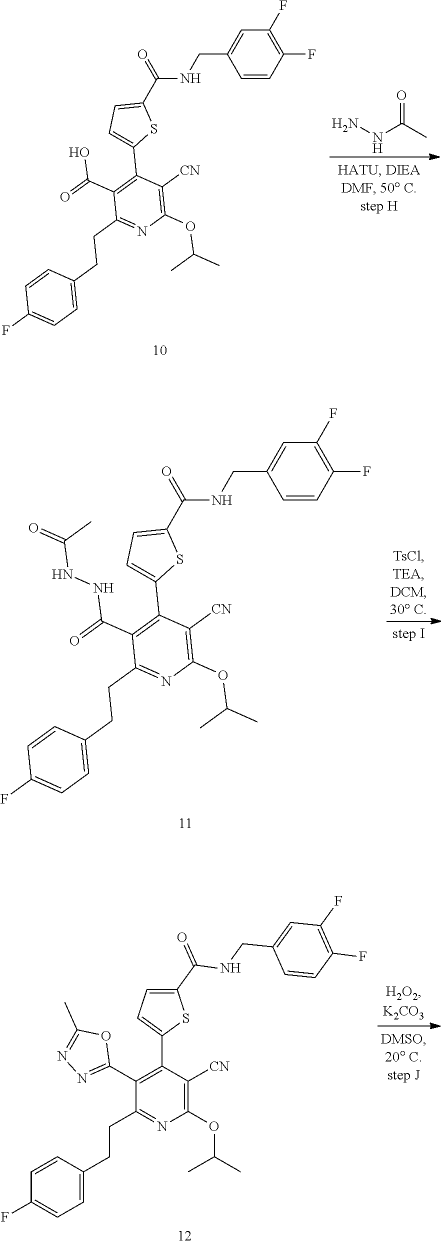

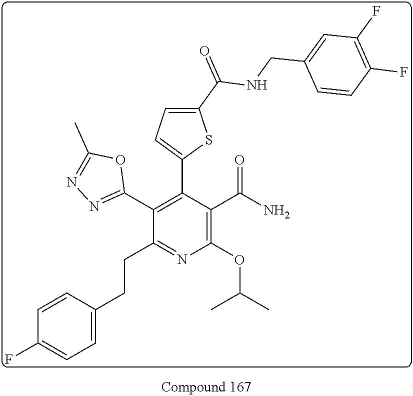 [SMILES: Cc1nnc(-c2c(CCc3ccc(F)cc3)nc(OC(C)C)c(C(N)=O)c2-c2ccc(C(=O)NCc3ccc(F)c(F)c3)s2)o1]

### Step A 5-(3-cyano-5-(ethoxycarbonyl)-6-(4-fluorophenethyl)-2-methoxy-1,4-dihydropyridin-4-yl)thiophene-2-carboxylic acid

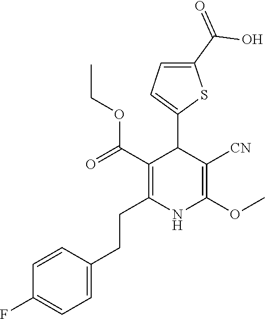 [SMILES: CCOC(=O)C1=C(CCc2ccc(F)cc2)NC(OC)=C(C#N)C1c1ccc(C(=O)O)s1]

To a solution of ethyl 5-(4-fluorophenyl)-3-oxopentanoate (3 g, 12.6 mmol) in MeOH (300 mL) was added NaOH (1.51 g, 37.8 mmol), 5-formylthiophene-2-carboxylic acid (1.97 g, 12.6 mmol) and propanedinitrile (832 mg, 12.6 mmol). The mixture was stirred at 20° C. for 3 hrs. The reaction mixture was concentrated under reduced pressure to remove solvent. The crude product 5-[5-cyano-3-ethoxycarbonyl-2-[2-(4-fluorophenyl)ethyl]-6-methoxy-1,4-dihydropyridin-4-yl]thiophene-2-carboxylic acid (5.75 g, 12.6 mmol, 100.00% yield) was obtained. LC-MS:m/z 455.4 (M−H)⁺

### Step B 5-[5-cyano-3-ethoxycarbonyl-2-[2-(4-fluorophenyl)ethyl]-6-methoxy-4-pyridyl]thiophene-2-carboxylic acid

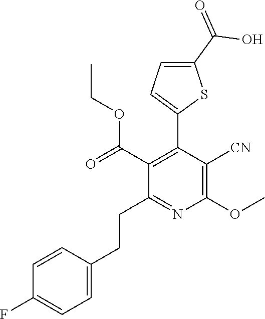 [SMILES: CCOC(=O)c1c(CCc2ccc(F)cc2)nc(OC)c(C#N)c1-c1ccc(C(=O)O)s1]

To a solution of 5-[5-cyano-3-ethoxycarbonyl-2-[2-(4-fluorophenyl)ethyl]-6-methoxy-1,4-dihydropyridin-4-yl]thiophene-2-carboxylic acid (5.75 g, 12.6 mmol) in MeCN (30 mL)/H₂O (10 mL) was added dipotassium sulfonatooxy sulfate (3.41 g, 12.6 mmol). The mixture was stirred at 85° C. for 2 hrs. The reaction mixture was concentrated under reduced pressure to remove solvent. The residue was purified by flash silica gel chromatography (ISCO®; 20 g SepaFlash® Silica Flash Column, Eluent of 0˜100% Ethyl acetate/Petroleum ethergradient @ 40 mL/min). 5-[5-cyano-3-ethoxycarbonyl-2-[2-(4-fluorophenyl)ethyl]-6-methoxy-4-pyridyl]thiophene-2-carboxylic acid (4 g, 7.04 mmol, 55.90% yield, 80% purity) was obtained. LC-MS:m/z 455.4 455.4 (M+H)⁺

¹H NMR (400 MHz, CDCl₃) δ ppm 7.14 (d, J=7.46 Hz, 3H), 6.92-7.00 (m, 3H), 3.88-4.14 (m, 6H), 3.04 (s, 3H), 0.86-1.05 (m, 3H).

### Step C 5-[5-cyano-3-ethoxycarbonyl-2-[2-(4-fluorophenyl)ethyl]-6-hydroxy-4-pyridyl]thiophene-2-carboxylic acid

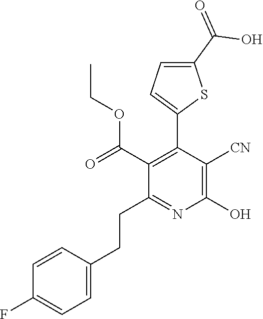 [SMILES: CCOC(=O)c1c(CCc2ccc(F)cc2)nc(O)c(C#N)c1-c1ccc(C(=O)O)s1]

To a solution of 5-[5-cyano-3-ethoxycarbonyl-2-[2-(4-fluorophenyl)ethyl]-6-methoxy-4-pyridyl]thiophene-2-carboxylic acid (3.8 g, 8.36 mmol) in HOAc (5 mL) was added HBr (786.7 mg, 4.18 mmol, 528 μL, 43% purity). The mixture was stirred at 0° C. for 2 hrs. The reaction mixture was adjusted pH to 7 and then diluted with H₂O (20 mL) and extracted with EtOAc (25 mL×3). The combined organic layers were washed with brine (30 mL), dried over Na₂SO₄, filtered and concentrated under reduced pressure to give a residue. The residue was purified by flash silica gel chromatography (ISCO®; 40 g SepaFlash® Silica Flash Column, Eluent of 0˜80% Ethyl acetate/Petroleum ethergradient @ 40 mL/min). 5-[5-cyano-3-ethoxycarbonyl-2-[2-(4-fluorophenyl)ethyl]-6-hydroxy-4-pyridyl]thiophene-2-carboxylic acid (3 g, 6.81 mmol, 81.46% yield) was obtained. LC-MS:m/z 455.4 441.4 (M+H)⁺

### Step D ethyl 5-cyano-4-(5-((3,4-difluorobenzyl)carbamoyl)thiophen-2-yl)-2-(4-fluorophenethyl)-6-hydroxynicotinate

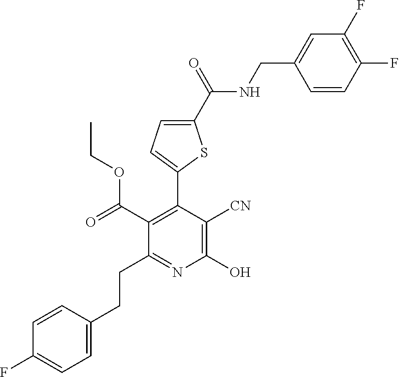 [SMILES: CCOC(=O)c1c(CCc2ccc(F)cc2)nc(O)c(C#N)c1-c1ccc(C(=O)NCc2ccc(F)c(F)c2)s1]

To a solution of 5-[5-cyano-3-ethoxycarbonyl-2-[2-(4-fluorophenyl)ethyl]-6-hydroxy-4-pyridyl]thiophene-2-carboxylic acid (1.7 g, 3.86 mmol) in DMF (20 mL) was added HATU (1.76 g, 4.63 mmol), (3,4-difluorophenyl)methanamine (552 mg, 3.86 mmol) and DIPEA (1.5 g, 11.6 mmol). The mixture was stirred at 20° C. for 3 hrs. The reaction mixture was diluted with H₂O (10 mL) and extracted with EtOAc (10 mL×3). The combined organic layers were washed with brine (25 mL×3), dried over Na₂SO₄, filtered and concentrated under reduced pressure to give a residue. The residue was purified by flash silica gel chromatography (ISCO®; 4 g SepaFlash® Silica Flash Column, Eluent of 0˜45% Ethyl acetate/Petroleum ethergradient @ 20 mL/min). ethyl 5-cyano-4-(5-((3,4-difluorobenzyl)carbamoyl)thiophen-2-yl)-2-(4-fluorophenethyl)-6-hydroxynicotinate (1.4 g, crude) was obtained.

### Step E ethyl 6-chloro-5-cyano-4-[5-[(3,4-difluorophenyl)methylcarbamoyl]-2-thienyl]-2-[2-(4-fluorophenyl)ethyl]pyridine-3-carboxylate

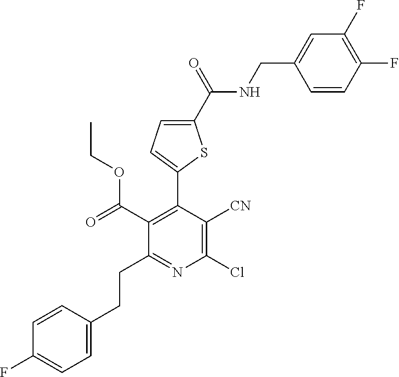 [SMILES: CCOC(=O)c1c(CCc2ccc(F)cc2)nc(Cl)c(C#N)c1-c1ccc(C(=O)NCc2ccc(F)c(F)c2)s1]

To a solution of ethyl 5-cyano-4-[5-[(3,4-difluorophenyl)methylcarbamoyl]-2-thienyl]-2-[2-(4-fluorophenyl)ethyl]-6-hydroxy-pyridine-3-carboxylate (1.7 g, 3.01 mmol) in MeCN (4 mL) was added POCl₃ (6.58 g, 42.9 mmol, 4 mL). The mixture was stirred at 80° C. for 2 hrs. The reaction mixture was concentrated under reduced pressure to remove solvent. The residue was purified by flash silica gel chromatography (ISCO®; 4 g SepaFlash® Silica Flash Column, Eluent of 0˜40% Ethyl acetate/Petroleum ethergradient @ 20 mL/min). ethyl 6-chloro-5-cyano-4-[5-[(3,4-difluorophenyl)methylcarbamoyl]-2-thienyl]-2-[2-(4-fluorophenyl)ethyl]pyridine-3-carboxylate (800 mg, 1.23 mmol, 41% yield, 90% purity) was obtained. LC-MS:m/z 584.5 (M+H)⁺

### Step F ethyl 5-cyano-4-[5-[(3,4-difluorophenyl)methylcarbamoyl]-2-thienyl]-2-[2-(4-fluorophenyl)ethyl]-6-isopropoxy-pyridine-3-carboxylate

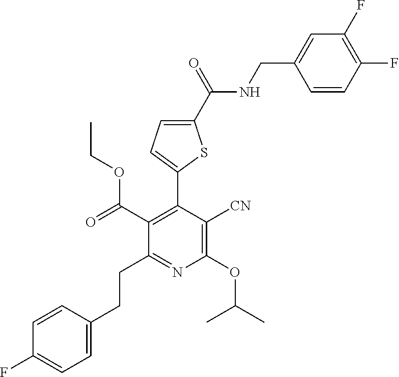 [SMILES: CCOC(=O)c1c(CCc2ccc(F)cc2)nc(OC(C)C)c(C#N)c1-c1ccc(C(=O)NCc2ccc(F)c(F)c2)s1]

To a solution of ethyl 6-chloro-5-cyano-4-[5-[(3,4-difluorophenyl)methylcarbamoyl]-2-thienyl]-2-[2-(4-fluorophenyl)ethyl]pyridine-3-carboxylate (300 mg, 514 μmol) in propan-2-ol (5 mL) was added Cs₂CO₃ (502 mg, 1.54 mmol). The mixture was stirred at 50° C. for 16 hrs. The reaction mixture was filtered and concentrated under reduced pressure to remove solvent. The crude product ethyl 5-cyano-4-[5-[(3,4-difluorophenyl)methylcarbamoyl]-2-thienyl]-2-[2-(4-fluorophenyl)ethyl]-6-isopropoxy-pyridine-3-carboxylate (312 mg) was obtained. LC-MS: m/z 608.6 (M+H)⁺.

### Step G 5-cyano-4-[5-[(3,4-difluorophenyl)methylcarbamoyl]-2-thienyl]-2-[2-(4-fluorophenyl)ethyl]-6-isopropoxy-pyridine-3-carboxylic acidoxamide

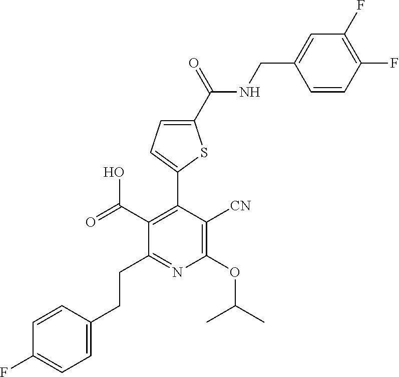 [SMILES: CC(C)Oc1nc(CCc2ccc(F)cc2)c(C(=O)O)c(-c2ccc(C(=O)NCc3ccc(F)c(F)c3)s2)c1C#N]

To a solution of ethyl 5-cyano-4-[5-[(3,4-difluorophenyl)methylcarbamoyl]-2-thienyl]-2-[2-(4-fluorophenyl)ethyl]-6-isopropoxy-pyridine-3-carboxylate (312 mg, 514 μmol) in DMA (2 mL) was added LiCl (109 mg, 2.57 mmol). The mixture was stirred at 120° C. for 16 hrs. The reaction mixture was diluted with H₂O (10 mL) and extracted with EtOAc (15 mL×3). The combined organic layers were washed with brine (50 mL×3), dried over Na₂SO₄, filtered and concentrated under reduced pressure to give a residue. The residue was purified by flash silica gel chromatography (ISCO®; 12 g SepaFlash® Silica Flash Column, Eluent of 0˜100% Ethyl acetate/Petroleum ethergradient @ 40 mL/min). 5-cyano-4-[5-[(3,4-difluorophenyl)methylcarbamoyl]-2-thienyl]-2-[2-(4-fluorophenyl)ethyl]-6-isopropoxy-pyridine-3-carboxylic acid (200 mg, 207 mol, 40.3% yield, 60% purity) was obtained. LC-MS: m/z 580.5 (M+H)⁺

### Step H 5-(3-(2-acetylhydrazine-1-carbonyl)-5-cyano-2-(4-fluorophenethyl)-6-isopropoxypyridin-4-yl)-N-(3,4-difluorobenzyl)thiophene-2-carboxamide

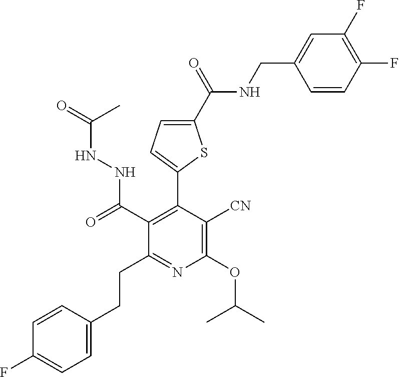 [SMILES: CC(=O)NNC(=O)c1c(CCc2ccc(F)cc2)nc(OC(C)C)c(C#N)c1-c1ccc(C(=O)NCc2ccc(F)c(F)c2)s1]

To a solution of 5-cyano-4-[5-[(3,4-difluorophenyl)methylcarbamoyl]-2-thienyl]-2-[2-(4-fluorophenyl)ethyl]-6-isopropoxy-pyridine-3-carboxylic acid (190 mg, 328 μmol) in DMF (2 mL) was added acetohydrazide (36.4 mg, 492 μmol), DIPEA (127 mg, 983 μmol) and HATU (150 mg, 393 μmol). The mixture was stirred at 20° C. for 16 hrs. The reaction mixture was concentrated under reduced pressure to remove solvent. The residue was purified by flash silica gel chromatography (ISCO®; 12 g SepaFlash® Silica Flash Column, Eluent of 0˜80% Ethyl acetate/Petroleum ethergradient @ 40 mL/min). Compound 5-(3-(2-acetylhydrazine-1-carbonyl)-5-cyano-2-(4-fluorophenethyl)-6-isopropoxypyridin-4-yl)-N-(3,4-difluorobenzyl)thiophene-2-carboxamide (208 mg, 328 μmol) was obtained. LC-MS: m/z 636.6 (M+H)⁺

### Step I 5-(3-cyano-6-(4-fluorophenethyl)-2-isopropoxy-5-(5-methyl-1,3,4-oxadiazol-2-yl)pyridin-4-yl)-N-(3,4-difluorobenzyl)thiophene-2-carboxamide

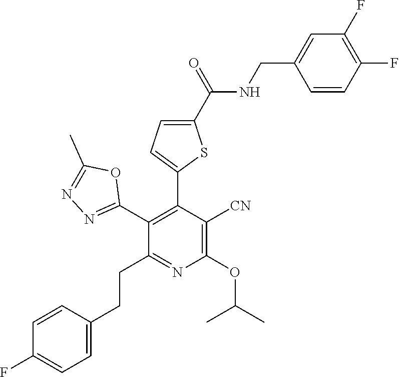 [SMILES: Cc1nnc(-c2c(CCc3ccc(F)cc3)nc(OC(C)C)c(C#N)c2-c2ccc(C(=O)NCc3ccc(F)c(F)c3)s2)o1]

To a solution of 5-[3-(acetamidocarbamoyl)-5-cyano-2-[2-(4-fluorophenyl)ethyl]-6-isopropoxy-4-pyridyl]-N-[(3,4-difluorophenyl)methyl]thiophene-2-carboxamide (208 mg, 328 μmol) in DCM (5 mL) was added 4-methylbenzenesulfonyl chloride (75 mg, 393 μmol) and TEA (99.5 mg, 983 μmol). The mixture was stirred at 50° C. for 3 hrs. The reaction mixture was concentrated under reduced pressure to remove solvent. The residue was purified by flash silica gel chromatography (ISCO®; 12 g SepaFlash® Silica Flash Column, Eluent of 0˜60% Ethyl acetate/Petroleum ethergradient @ 40 mL/min). Compound 5-(3-cyano-6-(4-fluorophenethyl)-2-isopropoxy-5-(5-methyl-1,3,4-oxadiazol-2-yl)pyridin-4-yl)-N-(3,4-difluorobenzyl)thiophene-2-carboxamide (81 mg, 131.14 μmol, 40.02% yield) was obtained. LC-MS: m/z 618.6 (M+H)⁺

### Step J 4-(5-((3,4-difluorobenzyl)carbamoyl)thiophen-2-yl)-6-(4-fluorophenethyl)-2-isopropoxy-5-(5-methyl-1,3,4-oxadiazol-2-yl)nicotinamide

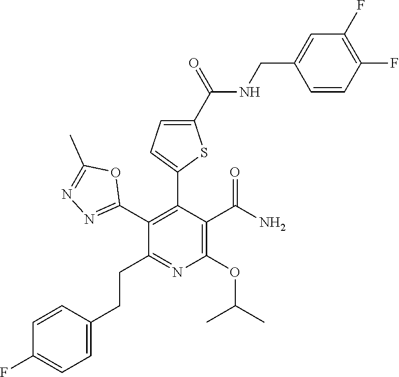 [SMILES: Cc1nnc(-c2c(CCc3ccc(F)cc3)nc(OC(C)C)c(C(N)=O)c2-c2ccc(C(=O)NCc3ccc(F)c(F)c3)s2)o1]

To a solution of 5-[5-cyano-2-[2-(4-fluorophenyl)ethyl]-6-isopropoxy-3-(5-methyl-1,3,4-oxadiazol-2-yl)-4-pyridyl]-N-[(3,4-difluorophenyl)methyl]thiophene-2-carboxamide (81 mg, 131 μmol) in DMSO (3 mL) was added H₂O₂ (78.7 mg, 1.97 mmol, 66.7 μL, 85% purity) and K₂CO₃ (36.2 mg, 262 μmol). The mixture was stirred at 20° C. for 16 hrs. The residue was diluted with H₂O (15 mL) and extracted with EtOAC (15 mL×3). The combined organic layer was washed with Na₂SO₃ (30 mL), brine (30 mL×3) dried over Na₂SO₄, filtered and concentrated under reduced pressure to give a residue. The residue was purified by prep-HPLC (Column: Kromasil 100-5-C18; Eluent: 45% to 80% water (0.1% FA)-ACN). 4-(5-((3,4-difluorobenzyl)carbamoyl)thiophen-2-yl)-6-(4-fluorophenethyl)-2-isopropoxy-5-(5-methyl-1,3,4-oxadiazol-2-yl)nicotinamide (11 mg, 17.30 μmol, 13.20% yield) was obtained.

¹H NMR (400 MHz, CDCl₃) δ 7.31-7.36 (m, 3H), 7.09-7.18 (m, 2H), 7.00-7.08 (m, 4H), 6.63 (br t, J=5.81 Hz, 1H), 5.49-5.68 (m, 2H), 5.44 (s, 2H), 4.49 (d, J=5.99 Hz, 2H), 2.75 (d, J=7.21 Hz, 2H), 2.50 (s, 3H), 2.32 (td, J=6.72, 13.45 Hz, 1H), 0.96 (d, J=6.60 Hz, 6H). LC-MS: m/z 658.3 (M+H)⁺.

### Example 30

### 4-(5-((3,4-difluorobenzyl)carbamoyl)thiophen-2-yl)-6-(4-fluorophenethyl)-2-(isopropylamino)-5-(5-methyl-1,3,4-oxadiazol-2-yl)nicotinamide (Compound 168)

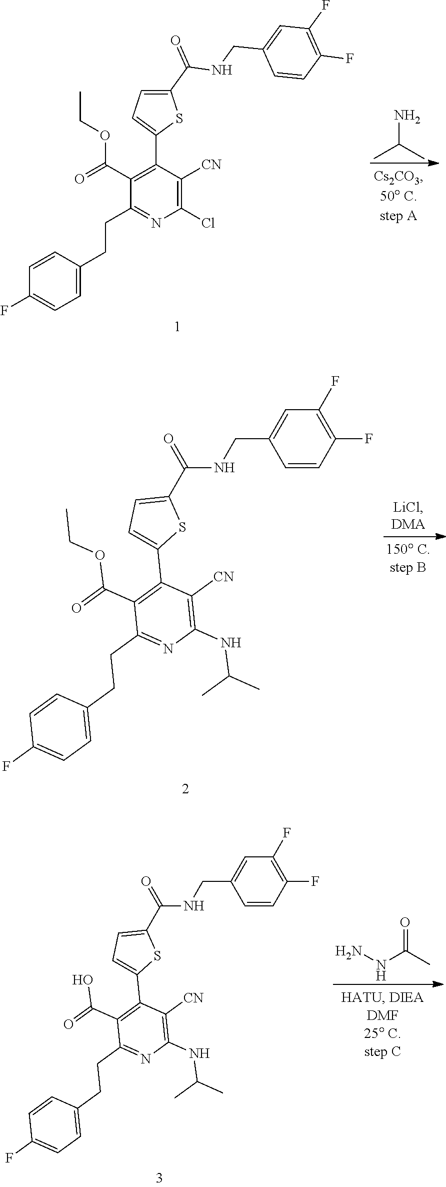

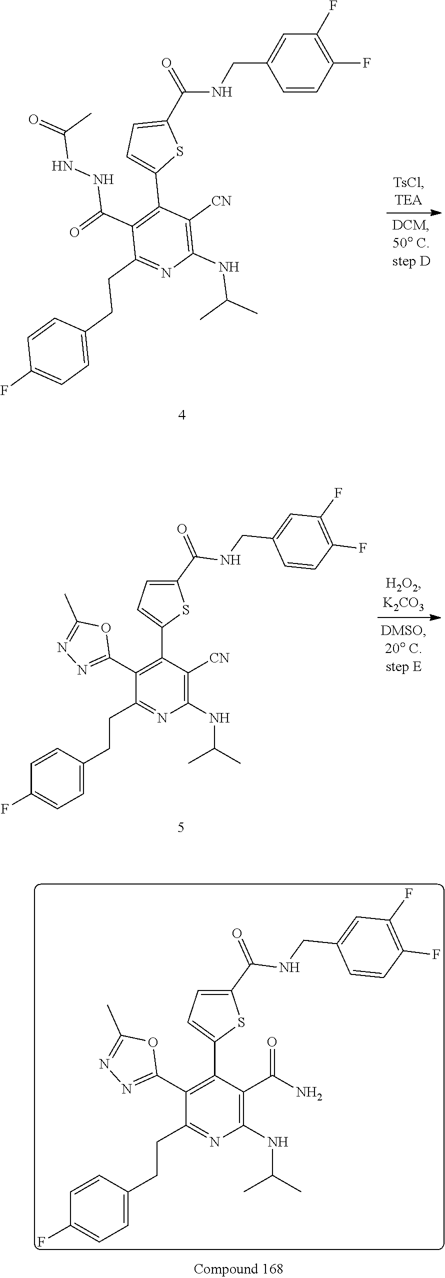 [SMILES: CC(=O)NNC(=O)c1c(CCc2ccc(F)cc2)nc(NC(C)C)c(C#N)c1-c1ccc(C(=O)NCc2ccc(F)c(F)c2)s1 ; Cc1nnc(-c2c(CCc3ccc(F)cc3)nc(NC(C)C)c(C#N)c2-c2ccc(C(=O)NCc3ccc(F)c(F)c3)s2)o1 ; Cc1nnc(-c2c(CCc3ccc(F)cc3)nc(NC(C)C)c(C(N)=O)c2-c2ccc(C(=O)NCc3ccc(F)c(F)c3)s2)o1]

### Step A ethyl 5-cyano-4-[5-[(3,4-difluorophenyl)methylcarbamoyl]-2-thienyl]-2-[2-(4-fluorophenyl)ethyl]-6-(isopropylamino)pyridine-3-carboxylate

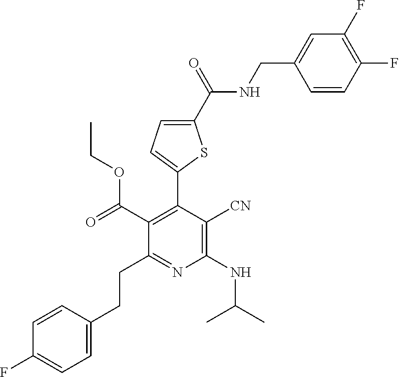 [SMILES: CCOC(=O)c1c(CCc2ccc(F)cc2)nc(NC(C)C)c(C#N)c1-c1ccc(C(=O)NCc2ccc(F)c(F)c2)s1]

To a solution of ethyl 6-chloro-5-cyano-4-[5-[(3,4-difluorophenyl)methylcarbamoyl]-2-thienyl]-2-[2-(4-fluorophenyl)ethyl]pyridine-3-carboxylate (200 mg, 342 μmol) in propan-2-amine (5 mL) was added Cs₂CO₃ (335 mg, 1.03 mmol). The mixture was stirred at 50° C. for 16 hrs. The reaction mixture was concentrated under reduced pressure to remove solvent. The residue was purified by flash silica gel chromatography (ISCO®; 12 g SepaFlash® Silica Flash Column, Eluent of 0˜35% Ethyl acetate/Petroleum ethergradient @ 40 mL/min). ethyl 5-cyano-4-[5-[(3,4-difluorophenyl)methylcarbamoyl]-2-thienyl]-2-[2-(4-fluorophenyl)ethyl]-6-(isopropylamino)pyridine-3-carboxylate (191 mg, 315 μmol, 91.9% yield) was obtained. LC-MS: m/z 607.6 (M+H)⁺

¹H NMR (400 MHz, CDCl₃) δ ppm 7.48 (d, J=3.91 Hz, 1H), 7.08-7.19 (m, 6H), 6.91-7.00 (m, 3H), 6.29-6.36 (m, 1H), 5.23 (d, J=7.34 Hz, 1H), 4.58 (d, J=5.99 Hz, 2H), 4.39 (dq, J=13.30, 6.69 Hz, 1H), 4.05 (q, J=7.09 Hz, 2H), 3.74-3.77 (m, 2H), 1.85 (t, J=3.36 Hz, 2H), 1.26-1.29 (m, 1H), 1.03 (t, J=7.15 Hz, 3H).

### Step B 5-cyano-4-[5-[(3,4-difluorophenyl)methylcarbamoyl]-2-thienyl]-2-[2-(4-fluorophenyl)ethyl]-6-(isopropylamino)pyridine-3-carboxylic acid

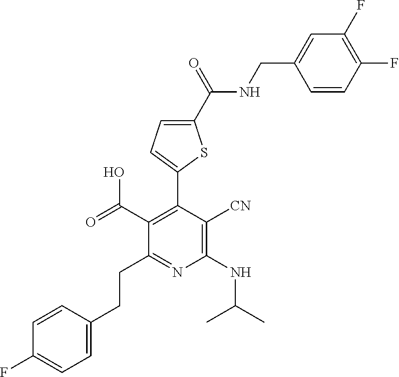 [SMILES: CC(C)Nc1nc(CCc2ccc(F)cc2)c(C(=O)O)c(-c2ccc(C(=O)NCc3ccc(F)c(F)c3)s2)c1C#N]

To a solution of ethyl 5-cyano-4-[5-[(3,4-difluorophenyl)methylcarbamoyl]-2-thienyl]-2-[2-(4-fluorophenyl)ethyl]-6-(isopropylamino)pyridine-3-carboxylate (171 mg, 282 μmol) in DMA (3 mL) was added LiCl (59.8 mg, 1.41 mmol, 28.9 μL). The mixture was stirred at 150° C. for 16 hrs. The reaction mixture was diluted with H₂O (10 mL) and extracted with EtOAC (15 mL×3). The combined organic layers were washed with brine (50 mL×3), dried over Na₂SO₄, filtered and concentrated under reduced pressure to give a residue. The residue was purified by flash silica gel chromatography (ISCO®; 12 g SepaFlash® Silica Flash Column, Eluent of 0˜100% Ethyl acetate/Petroleum ethergradient @ 40 mL/min). 5-cyano-4-[5-[(3,4-difluorophenyl)methylcarbamoyl]-2-thienyl]-2-[2-(4-fluorophenyl)ethyl]-6-(isopropylamino)pyridine-3-carboxylic acid (150 mg, 207 μmol, 73.6% yield, 80% purity) was obtained. LC-MS: m/z 579.5 (M+23H)⁺

### Step C 5-[3-(acetamidocarbamoyl)-5-cyano-2-[2-(4-fluorophenyl)ethyl]-6-(isopropylamino)-4-pyridyl]-N-[(3,4-difluorophenyl)methyl]thiophene-2-carboxamide

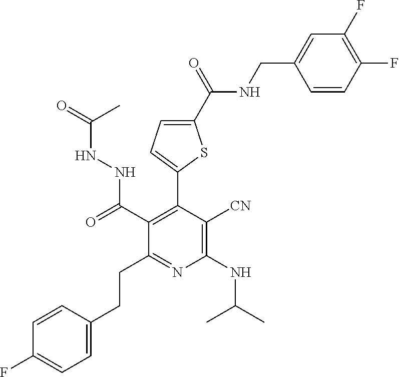 [SMILES: CC(=O)NNC(=O)c1c(CCc2ccc(F)cc2)nc(NC(C)C)c(C#N)c1-c1ccc(C(=O)NCc2ccc(F)c(F)c2)s1]

To a solution of 5-cyano-4-[5-[(3,4-difluorophenyl)methylcarbamoyl]-2-thienyl]-2-[2-(4-fluorophenyl)ethyl]-6-(isopropylamino)pyridine-3-carboxylic acid (150 mg, 259 μmol) in DMF (2 mL) was added acetohydrazide (28.8 mg, 389 μmol), DIPEA (100 mg, 778 μmol) and HATU (118 mg, 311 μmol). The mixture was stirred at 20° C. for 16 hrs. The reaction mixture was diluted with H₂O (10 mL) and extracted with EtOAC (15 mL×3). The combined organic layers were washed with brine (50 mL×3), dried over Na₂SO₄, filtered and concentrated under reduced pressure to give crude product 5-[3-(acetamidocarbamoyl)-5-cyano-2-[2-(4-fluorophenyl)ethyl]-6-(isopropylamino)-4-pyridyl]-N-[(3,4-difluorophenyl)methyl]thiophene-2-carboxamide (164 mg). LC-MS: m/z 635.6 (M+H)⁺

### Step D N 5-[5-cyano-2-[2-(4-fluorophenyl)ethyl]-6-(isopropylamino)-3-(5-methyl-1,3,4-oxadiazol-2-yl)-4-pyridyl]-N-[(3,4-difluorophenyl)methyl]thiophene-2-carboxamide

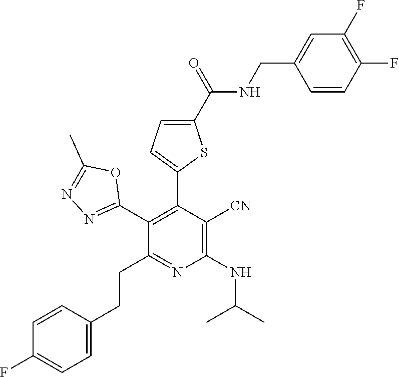 [SMILES: Cc1nnc(-c2c(CCc3ccc(F)cc3)nc(NC(C)C)c(C#N)c2-c2ccc(C(=O)NCc3ccc(F)c(F)c3)s2)o1]

To a solution of 5-[3-(acetamidocarbamoyl)-5-cyano-2-[2-(4-fluorophenyl)ethyl]-6-(isopropylamino)-4-pyridyl]-N-[(3,4-difluorophenyl)methyl]thiophene-2-carboxamide (164 mg, 259 μmol) in DCM (5 mL) was added 4-methylbenzenesulfonyl chloride (59.3 mg, 311 μmol) and TEA (78.7 mg, 777 μmol). The mixture was stirred at 50° C. for 3 hrs. The reaction mixture was concentrated under reduced pressure to remove solvent. The residue was purified by flash silica gel chromatography (ISCO®; 12 g SepaFlash® Silica Flash Column, Eluent of 0˜60% Ethyl acetate/Petroleum ethergradient @ 40 mL/min). 5-[5-cyano-2-[2-(4-fluorophenyl)ethyl]-6-(isopropylamino)-3-(5-methyl-1,3,4-oxadiazol-2-yl)-4-pyridyl]-N-[(3,4-difluorophenyl)methyl]thiophene-2-carboxamide (137 mg, 222 μmol, 85.7% yield) was obtained. LC-MS: m/z 617.6 (M+H)⁺

### Step E 4-(5-((3,4-difluorobenzyl)carbamoyl)thiophen-2-yl)-6-(4-fluorophenethyl)-2-(isopropylamino)-5-(5-methyl-1,3,4-oxadiazol-2-yl)nicotinamide

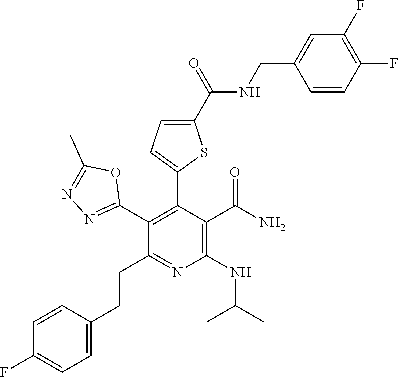 [SMILES: Cc1nnc(-c2c(CCc3ccc(F)cc3)nc(NC(C)C)c(C(N)=O)c2-c2ccc(C(=O)NCc3ccc(F)c(F)c3)s2)o1]

To a solution of 5-[5-cyano-2-[2-(4-fluorophenyl)ethyl]-6-(isopropylamino)-3-(5-methyl-1,3,4-oxadiazol-2-yl)-4-pyridyl]-N-[(3,4-difluorophenyl)methyl]thiophene-2-carboxamide (137 mg, 222 μmol) in DMSO (3 mL) was added H₂O₂ (133 mg, 3.33 mmol, 833 μL, 85% purity) and K₂CO₃ (61.4 mg, 444 μmol). The mixture was stirred at 20° C. for 16 hrs. The residue was diluted with H₂O (15 mL) and extracted with EtOAC (15 mL×3). The combined organic layers were washed with Na₂SO₃ (30 mL), brine (30 mL×3), dried over Na₂SO₄, filtered and concentrated under reduced pressure to give a residue. The residue was purified by prep-HPLC (Column: Kromasil 100-5-C18; Eluent: 45% to 80% water (0.1% FA)-ACN). 4-(5-((3,4-difluorobenzyl)carbamoyl)thiophen-2-yl)-6-(4-fluorophenethyl)-2-(isopropylamino)-5-(5-methyl-1,3,4-oxadiazol-2-yl)nicotinamide (58 mg, 91.4 μmol, 41.1% yield, 100% purity) was obtained.

¹H NMR (400 MHz, CDCl₃) δ ppm 7.33 (d, J=3.67 Hz, 1H), 7.01-7.17 (m, 6H), 6.87-6.94 (m, 3H), 6.79 (d, J=7.21 Hz, 1H), 6.60-6.65 (m, 1H), 5.61 (s, 1H), 5.33 (s, 1H), 4.50 (d, J=5.75 Hz, 2H), 4.39 (dq, J=12.84, 6.52 Hz, 1H), 2.97-3.03 (m, 2H), 2.85-2.91 (m, 2H), 2.34 (s, 3H), 1.28 (d, J=6.48 Hz, 6H). LC-MS: m/z 635.3 (M+H)⁺.

### Example 31

### 4-(5-((3,4-difluorobenzyl)carbamoyl)thiophen-2-yl)-6-(4-fluorophenethyl)-2-isobutyl-5-(2-methyloxazol-4-yl)nicotinamide (Compound 169)

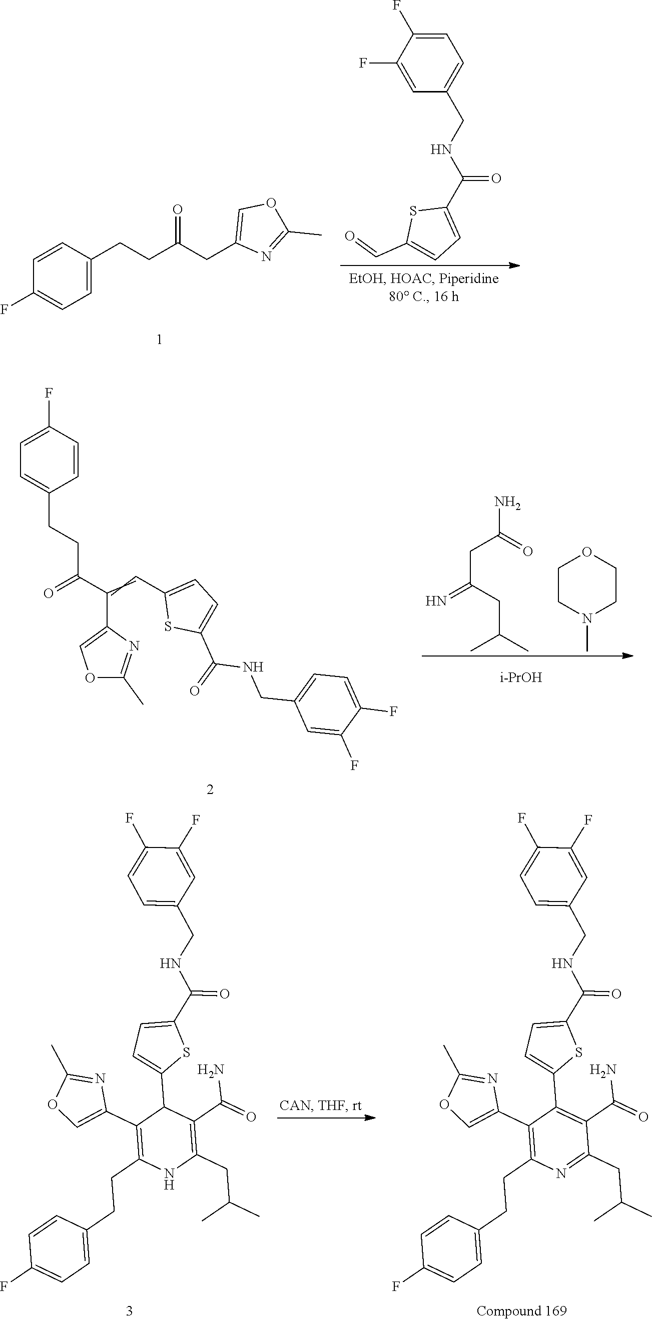

### Step A N-(3,4-difluorobenzyl)-5-(5-(4-fluorophenyl)-2-(2-methyloxazol-4-yl)-3-oxopent-1-en-1-yl)thiophene-2-carboxamide

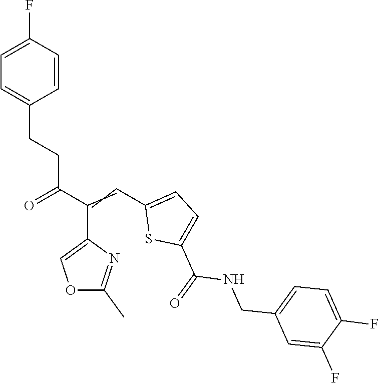 [SMILES: Cc1nc(C(=Cc2ccc(C(=O)NCc3ccc(F)c(F)c3)s2)C(=O)CCc2ccc(F)cc2)co1]

A mixture of 4-(4-fluorophenyl)-1-(2-methyloxazol-4-yl)butan-2-one (70 mg, 0.28 mmol), N-(3,4-difluorobenzyl)-5-formylthiophene-2-carboxamide (78.68 mg, 0.28 mmol), piperidine (1.19 mg, 0.014 mmol), HOAc (0.85 mg, 0.014 mmol) in EtOH (2 mL) was stirred at 80° C. for overnight. After the reaction was completed, the mixture was filtered and concentrated to give N-(3,4-difluorobenzyl)-5-(5-(4-fluorophenyl)-2-(2-methyloxazol-4-yl)-3-oxopent-1-en-1-yl)thiophene-2-carboxamide (205 mg, crude). LC-MS: m/z 511.3 [M+H]⁺.

### 4-(5-((3,4-difluorobenzyl)carbamoyl)thiophen-2-yl)-6-(4-fluorophenethyl)-2-isobutyl-5-(2-methyloxazol-4-yl)nicotinamide

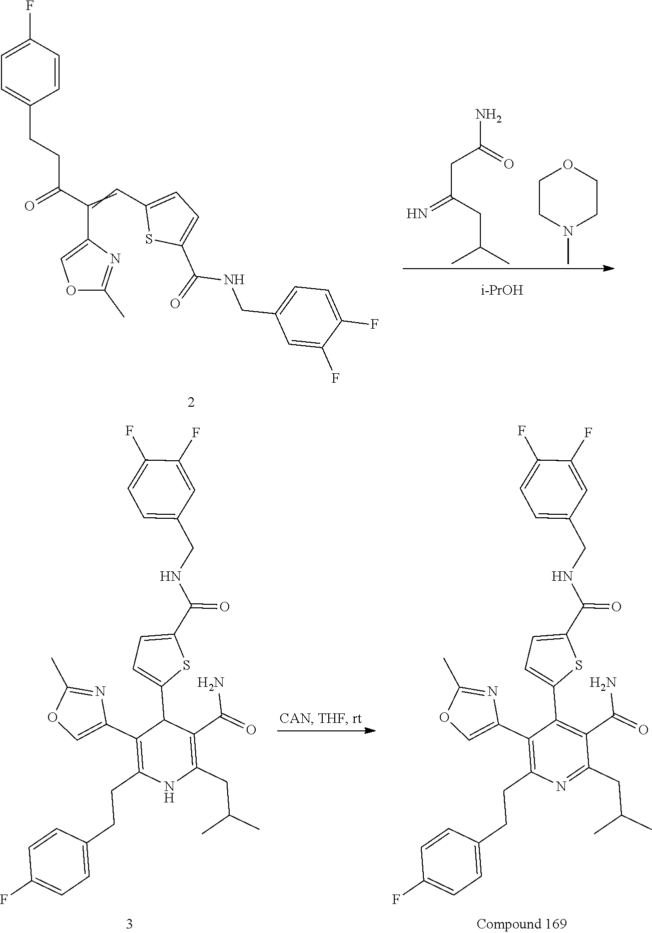

A mixture of N-(3,4-difluorobenzyl)-5-(5-(4-fluorophenyl)-2-(2-methyloxazol-4-yl)-3-oxopent-1-en-1-yl)thiophene-2-carboxamide (100 mg, crude), 3-imino-5-methylhexanamide (38.98 mg, 0.27 mmol), 4-methylmorpholine (19.8 mg, 0.20 mmol) in i-PrOH (3 mL) was stirred at 100° C. for overnight. After the reaction was completed, the mixture was diluted with EA (60 mL), washed with water (50 mL). The organic layers were dried over Na₂SO₄, filtered and concentrated to give 4-(5-((3,4-difluorobenzyl)carbamoyl)thiophen-2-yl)-6-(4-fluorophenethyl)-2-isobutyl-5-(2-methyloxazol-4-yl)-1,4-dihydropyridine-3-carboxamide (50 mg, crude). To a solution of 4-(5-((3,4-difluorobenzyl)carbamoyl)thiophen-2-yl)-6-(4-fluorophenethyl)-2-isobutyl-5-(2-methyloxazol-4-yl)-1,4-dihydropyridine-3-carboxamide (50 mg, crude) in THF (2 mL) was added CAN (86.47 mg, 0.16 mmol), and the reaction was stirred at room temperature for 12 hr. After the reaction was completed, the mixture was diluted with EA (50 mL), washed with water (40 mL). The organic layers were dried over Na₂SO₄, filtered and concentrated under vacuum to dryness. The residue was purified by column chromatography on C18-25 g (FA+0.1%) to give 4-(5-((3,4-difluorobenzyl)carbamoyl)thiophen-2-yl)-6-(4-fluorophenethyl)-2-isobutyl-5-(2-methyloxazol-4-yl)nicotinamide (6.31 mg, 12.67%).

¹H NMR (400 MHz, DMSO-d6): δ 9.06 (t, J=6.0 Hz, 1H), 7.77 (br s, 1H), 7.59-7.64 (m, 2H), 7.31-7.44 (m, 3H), 7.02-7.19 (m, 5H), 6.95 (d, J=3.6 Hz, 1H), 4.39 (d, J=6.0 Hz, 2H), 2.92 (s, 4H), 2.66 (d, J=7.2 Hz, 2H), 2.40 (s, 3H), 2.22-2.32 (m, 1H), 0.92 (d, J=6.4 Hz, 6H). 19F NMR (377 MHz, DMSO-d6): −117.50, −138.88, −141.39. LC-MS: m/z 633.3 [M+H]⁺.

### Example 32

### 4-(3-((3,4-difluorobenzyl)carbamoyl)pyrrolidin-1-yl)-6-(4-fluorophenethyl)-2-isobutyl-5-(5-methyl-1,3,4-oxadiazol-2-yl)nicotinamide (Compound 170)

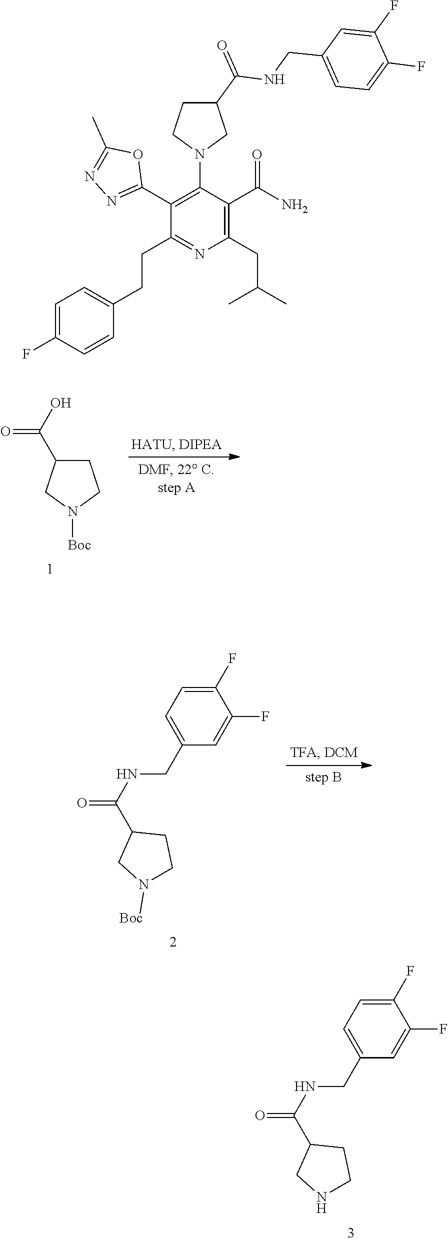 [SMILES: Cc1nnc(-c2c(CCc3ccc(F)cc3)nc(CC(C)C)c(C(N)=O)c2N2CCC(C(=O)NCc3ccc(F)c(F)c3)C2)o1 ; CC(C)(C)OC(=O)N1CCC(C(=O)O)C1 ; CC(C)(C)OC(=O)N1CCC(C(=O)NCc2ccc(F)c(F)c2)C1 ; O=C(NCc1ccc(F)c(F)c1)C1CCNC1]

### Step A tert-butyl 3-((3,4-difluorobenzyl)carbamoyl)pyrrolidine-1-carboxylate

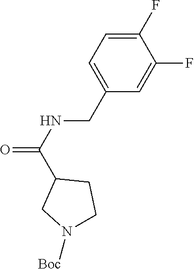 [SMILES: CC(C)(C)OC(=O)N1CCC(C(=O)NCc2ccc(F)c(F)c2)C1]

To a solution of 1-tert-butoxycarbonylpyrrolidine-3-carboxylic acid (2 g, 9.29 mmol) in DMF (40 mL) was added HATU (3.5 g, 9.29 mmol) and DIEA (1.20 g, 9.29 mmol). The mixture was stirred at 0° C. for 30 min. Then (3,4-difluorophenyl)methanamine (1.33 g, 9.29 mmol) was added in one portion. The mixture was stirred at 22° C. for 16 hrs. The reaction mixture was diluted with H₂O (100 mL) and extracted with EA (50 mL). The organic layer was washed with brine (100 mL), dried over Na₂SO₄, filtered and concentrated under reduced pressure to give a residue. The crude product was purified by column chromatography on silica gel (eluted with 0˜50% EA in PE). tert-butyl 3-[(3,4-difluorophenyl)methylcarbamoyl]pyrrolidine-1-carboxylate (2.2 g, 5.82 mmol) was obtained.

¹H NMR (400 MHz, CDCl₃) δ ppm 7.05-7.17 (m, 2H), 7.00 (s, 1H), 5.94 (s, 1H), 4.41 (d, J=5.75 Hz, 2H), 3.47-3.69 (m, 3H), 3.28-3.40 (m, 1H), 2.88 (d, J=5.87 Hz, 1H), 2.06-2.26 (m, 2H), 1.46 (s, 9H). LC-MS: m/z 363.4 (M+23)⁺.

### Step B N-(3,4-difluorobenzyl)pyrrolidine-3-carboxamide

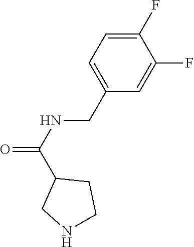 [SMILES: O=C(NCc1ccc(F)c(F)c1)C1CCNC1]

A mixture of tert-butyl 3-[(3,4-difluorophenyl)methylcarbamoyl]pyrrolidine-1-carboxylate (1 g, 2.94 mmol) in TFA (6.14 g, 53.85 mmol, 4.00 mL) and DCM (18 mL) was degassed and purged with N₂ for 3 times, and then the mixture was stirred at 22° C. for 5 hrs under N₂ atmosphere. The reaction mixture was concentrated under reduced pressure to remove solvent. The residue was diluted with EtOAc and washed with aqueous NaHCO₃ (50 mL×2) and brine (50 mL), dried over Na₂SO₄, filtered and concentrated under reduced pressure to give crude N-(3,4-difluorobenzyl)pyrrolidine-3-carboxamide (553 mg).

¹H NMR (400 MHz, DMSO-d₆) δ ppm 8.75 (t, J=5.62 Hz, 1H), 7.27-7.52 (m, 2H), 7.11 (ddd, J=6.08, 4.07, 2.20 Hz, 1H), 4.28 (d, J=5.87 Hz, 2H), 3.08-3.40 (m, 7H), 2.12-2.23 (m, 1H), 1.88-1.98 (m, 1H).

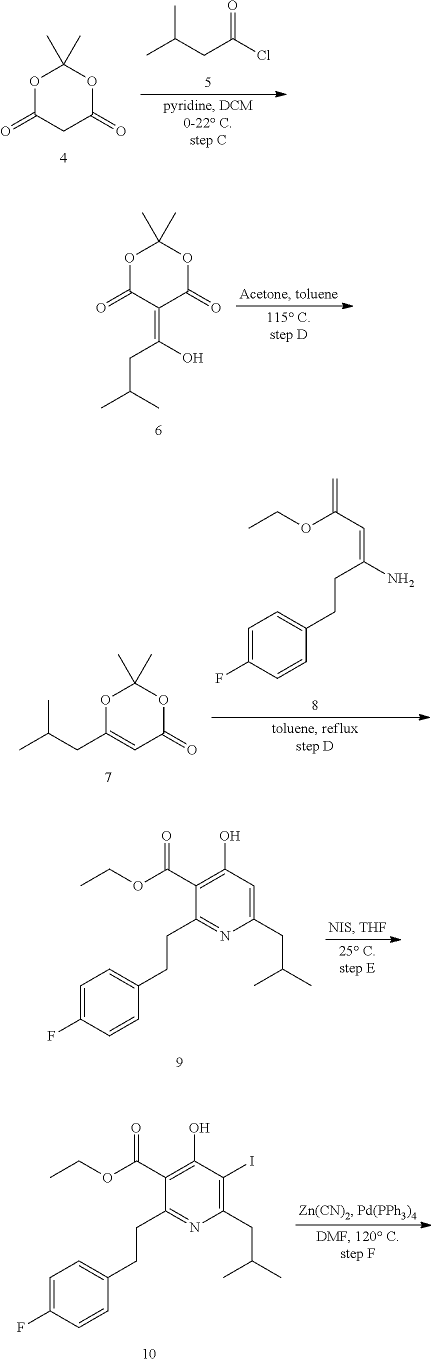

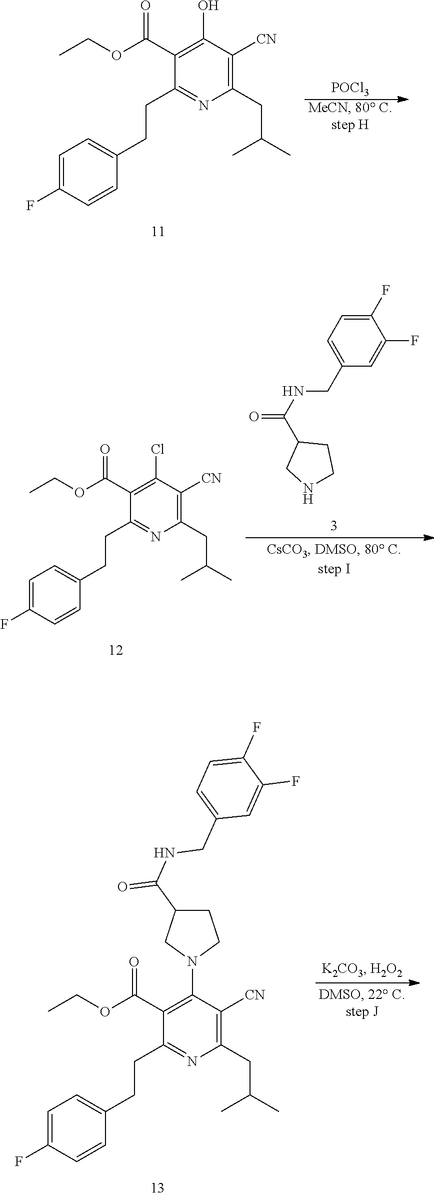 [SMILES: CCOC(=O)c1c(CCc2ccc(F)cc2)nc(CC(C)C)c(C#N)c1O ; "POCl3" ; "MeCN, 80° C.step H" ; CCOC(=O)c1c(CCc2ccc(F)cc2)nc(CC(C)C)c(C#N)c1Cl ; O=C(NCc1ccc(F)c(F)c1)C1CCNC1 ; CCOC(=O)c1c(CCc2ccc(F)cc2)nc(CC(C)C)c(C#N)c1N1CCC(C(=O)NCc2ccc(F)c(F)c2)C1]

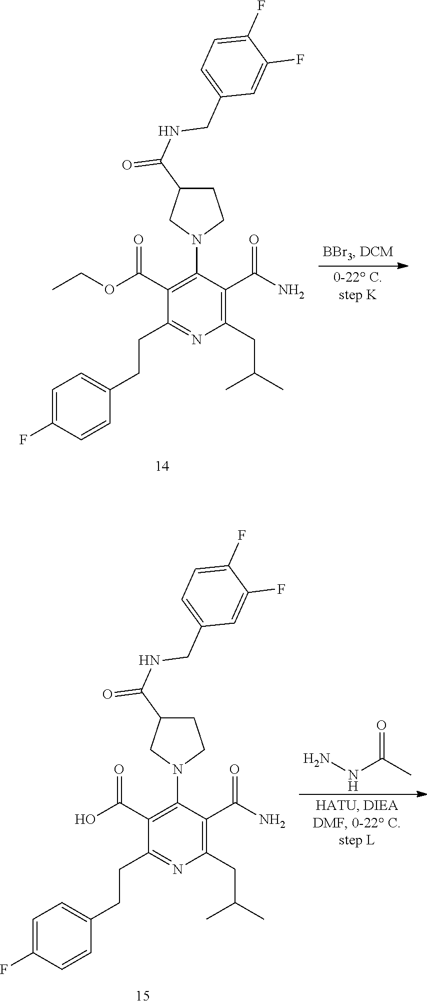

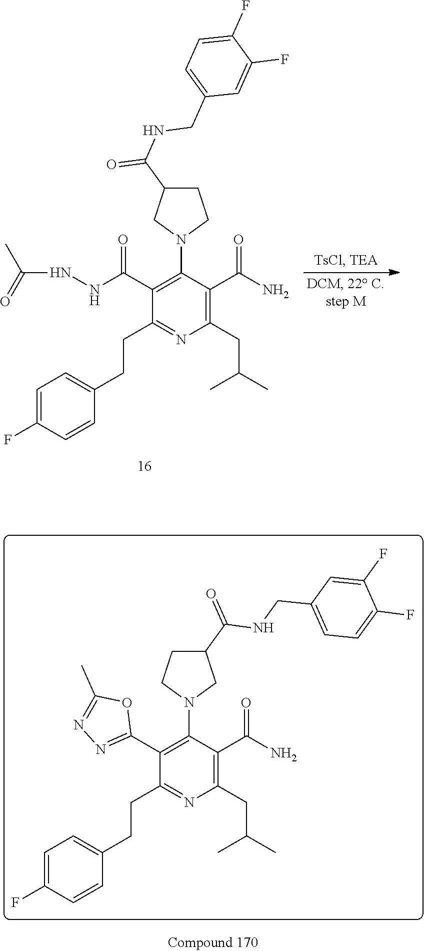 [SMILES: CC(=O)NNC(=O)c1c(CCc2ccc(F)cc2)nc(CC(C)C)c(C(N)=O)c1N1CCC(C(=O)NCc2ccc(F)c(F)c2)C1 ; Cc1nnc(-c2c(CCc3ccc(F)cc3)nc(CC(C)C)c(C(N)=O)c2N2CCC(C(=O)NCc3ccc(F)c(F)c3)C2)o1]

### Step C 5-(1-hydroxy-3-methylbutylidene)-2,2-dimethyl-1,3-dioxane-4,6-dione

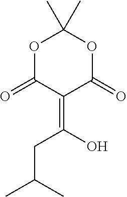 [SMILES: CC(C)CC(O)=C1C(=O)OC(C)(C)OC1=O]

To a solution of 2,2-dimethyl-1,3-dioxane-4,6-dione (10 g, 69.38 mmol) in DCM (40 mL) was added dropwise pyridine (16.46 g, 208.15 mmol, 17 mL) at 0° C. over 5 min. After addition, the mixture was stirred at this temperature for 1 hr, and then 3-methylbutanoyl chloride (10 g, 83.26 mmol) was added dropwise at 0° C. The resulting mixture was stirred at 22° C. for 16 hrs. The reaction mixture was partitioned between EA (200 mL) and water (100 mL). The organic phase was separated, washed with brine (100 mL), dried over Na₂SO₄, filtered and concentrated under reduced pressure to give a residue. The residue was purified by column chromatography on silica gel (eluted with 0˜30% EA in PE). 5-(1-hydroxy-3-methyl-butylidene)-2,2-dimethyl-1,3-dioxane-4,6-dione (14.7 g, 57.97 mmol) was obtained.

¹H NMR (400 MHz, CDCl₃) δ ppm 15.30 (s, 1H), 2.98 (d, J=7.09 Hz, 1H), 2.95-3.02 (m, 1H), 2.19-2.26 (m, 1H), 1.73 (s, 6H), 1.02 (d, J=6.60 Hz, 6H).

### Step D 6-isobutyl-2,2-dimethyl-4H-1,3-dioxin-4-one

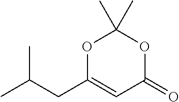

A mixture of 5-(1-hydroxy-3-methyl-butylidene)-2,2-dimethyl-1,3-dioxane-4,6-dione (14 g, 61.78 mmol) in toluene (140 mL) and acetone (14 mL) was degassed and purged with N₂ for 3 times, and then the mixture was stirred at 115° C. for 16 hrs under N₂ atmosphere. The reaction mixture was concentrated under reduced pressure to give 6-isobutyl-2,2-dimethyl-1,3-dioxin-4-one (9.7 g, 47.39 mmol).

¹H NMR (400 MHz, CDCl₃) δ ppm 5.13-5.30 (m, 1H), 2.09 (d, J=7.09 Hz, 2H), 1.91-2.01 (m, 1H), 1.69 (s, 6H), 0.94-0.98 (m, 6H).

### Step E ethyl 2-(4-fluorophenethyl)-4-hydroxy-6-isobutylnicotinate

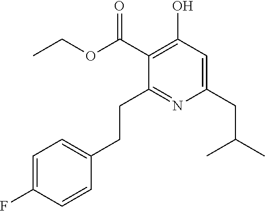 [SMILES: CCOC(=O)c1c(O)cc(CC(C)C)nc1CCc1ccc(F)cc1]

A mixture of 6-isobutyl-2,2-dimethyl-1,3-dioxin-4-one (3.11 g, 16.86 mmol), ethyl (E)-3-amino-5-(4-fluorophenyl)pent-2-enoate (2 g, 8.43 mmol) in toluene (60 mL) was degassed and purged with N₂ for 3 times, and then the mixture was stirred at 120° C. for 16 hrs under N₂ atmosphere. The reaction mixture was concentrated under reduced pressure to remove solvent. The crude product was purified by column chromatography on silica gel (eluted with 0˜50% EA in PE). ethyl 2-[2-(4-fluorophenyl)ethyl]-4-hydroxy-6-isobutyl-pyridine-3-carboxylate (578 mg, 1.51 mmol) was obtained. LC-MS: m/z 346.5 (M+H)⁺.

### Step F ethyl 2-(4-fluorophenethyl)-4-hydroxy-5-iodo-6-isobutylnicotinate

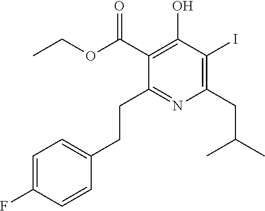 [SMILES: CCOC(=O)c1c(CCc2ccc(F)cc2)nc(CC(C)C)c(I)c1O]

A mixture of ethyl 2-[2-(4-fluorophenyl)ethyl]-4-hydroxy-6-isobutyl-pyridine-3-carboxylate (570 mg, 1.65 mmol), NIS (371 mg, 1.65 mmol) in THF (10 mL) was degassed and purged with N₂ for 3 times, and then the mixture was stirred at 25° C. for 4 hrs under N₂ atmosphere. The reaction mixture was concentrated under reduced pressure to remove solvent. The crude product was triturated with EA (3 mL) at 25° C. for 30 min. Ethyl 2-[2-(4-fluorophenyl)ethyl]-4-hydroxy-5-iodo-6-isobutyl-pyridine-3-carboxylate (670 mg, 995.11 μmol) was obtained. LC-MS: m/z 472.4 (M+H)⁺

### Step G ethyl 5-cyano-2-(4-fluorophenethyl)-4-hydroxy-6-isobutylnicotinate

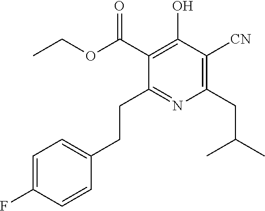 [SMILES: CCOC(=O)c1c(CCc2ccc(F)cc2)nc(CC(C)C)c(C#N)c1O]

A mixture of ethyl 2-[2-(4-fluorophenyl)ethyl]-4-hydroxy-5-iodo-6-isobutyl-pyridine-3-carboxylate (270 mg, 572.88 μmol), Zn(CN)₂ (0.260 g, 2.21 mmol), Pd(PPh₃)₄ (33 mg, 28.64 μmol) in DMF (5 mL) was degassed and purged with N₂ for 3 times, and then the mixture was stirred at 120° C. for 16 hrs under N₂ atmosphere. The reaction mixture was diluted with H₂O (100 mL) and extracted with EA (100 mL×2). The combined organic layers were washed with brine (100 mL), dried over Na₂SO₄, filtered and concentrated under reduced pressure to give a residue. The crude product was purified by column chromatography on silica gel (eluted with 0˜50% EA in PE). ethyl 5-cyano-2-[2-(4-fluorophenyl)ethyl]-4-hydroxy-6-isobutyl-pyridine-3-carboxylate (65 mg, 166.70 μmol) was obtained. LC-MS: m/z 371.5 (M+H)⁺.

### Step H ethyl 4-chloro-5-cyano-2-(4-fluorophenethyl)-6-isobutylnicotinate

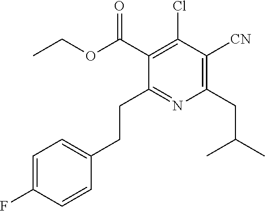 [SMILES: CCOC(=O)c1c(CCc2ccc(F)cc2)nc(CC(C)C)c(C#N)c1Cl]

To a solution of ethyl 5-cyano-2-[2-(4-fluorophenyl)ethyl]-4-hydroxy-6-isobutyl-pyridine-3-carboxylate (200 mg, 539.93 μmol) in MeCN (4 mL) was added POCl₃ (82.79 mg, 539.93 μmo). The mixture was stirred at 80° C. for 2 hrs. The reaction mixture was quenched by addition aqueous NaHCO₃ (50 mL) at 0° C., and then diluted with water (20 mL) and extracted with EA (50 mL). The combined organic layer was washed with brine (40 mL), dried over Na₂SO₄, filtered and concentrated under reduced pressure to give a residue. The residue was purified by column chromatography on silica gel (eluted with 0˜50% EA in PE). ethyl 4-chloro-5-cyano-2-[2-(4-fluorophenyl)ethyl]-6-isobutyl-pyridine-3-carboxylate (163 mg, 398.21 μmol) was obtained. LC-MS: m/z 389.5 (M+H)⁺.

### Step I ethyl 5-cyano-4-(3-((3,4-difluorobenzyl)carbamoyl)pyrrolidin-1-yl)-2-(4-fluorophenethyl)-6-isobutylnicotinate

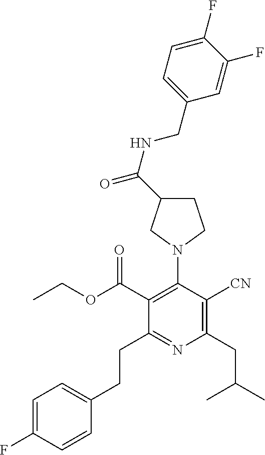 [SMILES: CCOC(=O)c1c(CCc2ccc(F)cc2)nc(CC(C)C)c(C#N)c1N1CCC(C(=O)NCc2ccc(F)c(F)c2)C1]

A mixture of ethyl 4-chloro-5-cyano-2-[2-(4-fluorophenyl)ethyl]-6-isobutyl-pyridine-3-carboxylate (150 mg, 385.74 μmol), N-[(3,4-difluorophenyl)methyl]pyrrolidine-3-carboxamide (111.21 mg, 462.89 μmol), Cs₂CO₃ (377.04 mg, 1.16 mmol) in DMSO (4 mL) was degassed and purged with N₂ for 3 times, and then the mixture was stirred at 80° C. for 16 hrs under N₂ atmosphere. The reaction mixture was concentrated under reduced pressure to remove solvent. The crude product was purified by column chromatography on silica gel (eluted with 0˜80% EA in PE) Compound ethyl 5-cyano-4-[3-[(3,4-difluorophenyl)methylcarbamoyl]pyrrolidin-1-yl]-2-[2-(4-fluorophenyl)ethyl]-6-isobutyl-pyridine-3-carboxylate (198 mg, 300.68 μmol) was obtained. LC-MS: m/z 593.6 (M+H)⁺.

### Step J ethyl 5-carbamoyl-4-(3-((3,4-difluorobenzyl)carbamoyl)pyrrolidin-1-yl)-2-(4-fluorophenethyl)-6-isobutylnicotinate

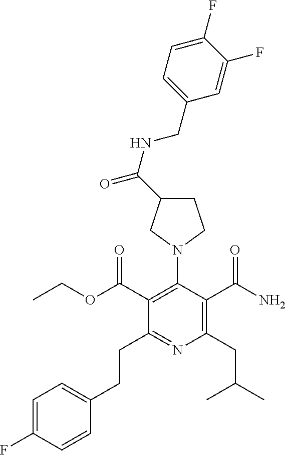 [SMILES: CCOC(=O)c1c(CCc2ccc(F)cc2)nc(CC(C)C)c(C(N)=O)c1N1CCC(C(=O)NCc2ccc(F)c(F)c2)C1]

A mixture of ethyl 5-cyano-4-[3-[(3,4-difluorophenyl)methylcarbamoyl]pyrrolidin-1-yl]-2-[2-(4-fluorophenyl)ethyl]-6-isobutyl-pyridine-3-carboxylate (140 mg, 236.23 μmol), K₂CO₃ (65.30 mg, 472.45 μmol) in DMSO (5 mL) and H₂O₂ (0.480 g, 4.66 mmol, 406.78 μL, 33% purity) was degassed and purged with N₂ for 3 times, and then the mixture was stirred at 22° C. for 16 hrs under N₂ atmosphere. The reaction mixture was quenched by addition aqueous Na₂S₂O₃ (40 mL) at 0° C., and then diluted with H₂O (100 mL) and extracted with EA (100 mL×2). The combined organic layers were washed with brine (100 mL), dried over Na₂SO₄, filtered and concentrated under reduced pressure to give a residue. The crude product was purified by column chromatography on silica gel (eluted with 0˜15% EA in PE). ethyl 5-carbamoyl-4-[3-[(3,4-difluorophenyl)methylcarbamoyl]pyrrolidin-1-yl]-2-[2-(4-fluorophenyl)ethyl]-6-isobutyl-pyridine-3-carboxylate (170 mg, 222.71 μmol) was obtained.

¹H NMR (400 MHz, CDCl₃) δ ppm 6.83-7.12 (m, 8H), 4.16-4.33 (m, 4H), 3.47-3.66 (m, 3H), 3.31 (br s, 1H), 2.81-3.02 (m, 6H), 2.60 (br d, J=7.09 Hz, 1H), 2.04-2.23 (m, 3H), 1.24-1.29 (m, 3H), 0.84 (dd, J=8.68, 6.72 Hz, 6H). LC-MS: m/z 611.7 (M+H)⁺.

### Step K 5-carbamoyl-4-(3-((3,4-difluorobenzyl)carbamoyl)pyrrolidin-1-yl)-2-(4-fluorophenethyl)-6-isobutylnicotinic acid

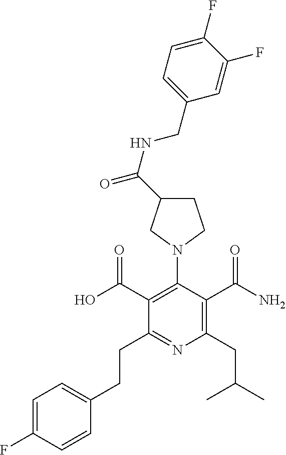 [SMILES: CC(C)Cc1nc(CCc2ccc(F)cc2)c(C(=O)O)c(N2CCC(C(=O)NCc3ccc(F)c(F)c3)C2)c1C(N)=O]

A mixture of ethyl 5-carbamoyl-4-[3-[(3,4-difluorophenyl)methylcarbamoyl]pyrrolidin-1-yl]-2-[2-(4-fluorophenyl)ethyl]-6-isobutyl-pyridine-3-carboxylate (100 mg, 163.76 μmol) in DCM (2 mL) was added BBr₃ (5.05 g, 20.14 mmol, 0.2 mL) by dropwise at 0° C., and then the mixture was stirred at 22° C. for 16 hrs under N₂ atmosphere. The reaction mixture was quenched by addition H₂O (50 mL) at 0° C., and then diluted with aq. NaHCO₃ (50 mL) and extracted with EA (50 mL×2). The combined organic layers were washed with H₂O (20 mL), dried over Na₂SO₄, filtered and concentrated under reduced pressure to give a residue. The residue was purified by prep-TLC (SiO₂, DCM:MeOH=10:1). 5-carbamoyl-4-[3-[(3,4-difluorophenyl)methylcarbamoyl]pyrrolidin-1-yl]-2-[2-(4-fluorophenyl)ethyl]-6-isobutyl-pyridine-3-carboxylic acid (69 mg, 94.75 μmol) was obtained. LC-MS: m/z 583.6 (M+H)⁺.

### Step L 1-(3-(2-acetylhydrazine-1-carbonyl)-5-cyano-2-(4-fluorophenethyl)-6-isobutylpyridin-4-yl)-N-(3,4-difluorobenzyl)pyrrolidine-3-carboxamide

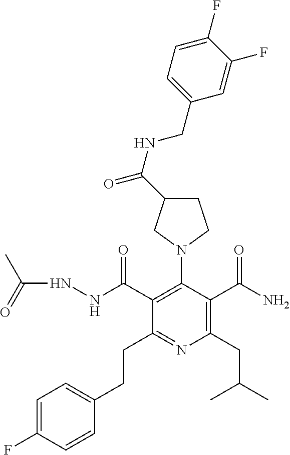 [SMILES: CC(=O)NNC(=O)c1c(CCc2ccc(F)cc2)nc(CC(C)C)c(C(N)=O)c1N1CCC(C(=O)NCc2ccc(F)c(F)c2)C1]

To a solution of 5-carbamoyl-4-[3-[(3,4-difluorophenyl)methylcarbamoyl]pyrrolidin-1-yl]-2-[2-(4-fluorophenyl)ethyl]-6-isobutyl-pyridine-3-carboxylic acid (50 mg, 85.82 μmol) in DMF (2 mL) was added HATU (35 mg, 92.05 μmol) and DIEA (74.20 mg, 574.13 mol, 0.1 mL). The mixture was stirred at 0° C. for 30 min. Then acetohydrazide (13 mg, 175.48 μmol) was added in one portion. The mixture was stirred at 0-22° C. for 12 hrs. The reaction mixture was partitioned between EA (50 mL) and water (50 mL). The organic phase was separated, washed with brine (30 mL), dried over Na₂SO₄, filtered and concentrated under reduced pressure to give crude 5-(acetamidocarbamoyl)-4-[3-[(3,4-difluorophenyl)methylcarbamoyl]pyrrolidin-1-yl]-6-[2-(4-fluorophenyl)ethyl]-2-isobutyl-pyridine-3-carboxamide (31 mg, 46.11 μmol). LC-MS: m/z 639.7 (M+H)⁺.

### Step M 4-(3-((3,4-difluorobenzyl)carbamoyl)pyrrolidin-1-yl)-6-(4-fluorophenethyl)-2-isobutyl-5-(5-methyl-1,3,4-oxadiazol-2-yl)nicotinamide

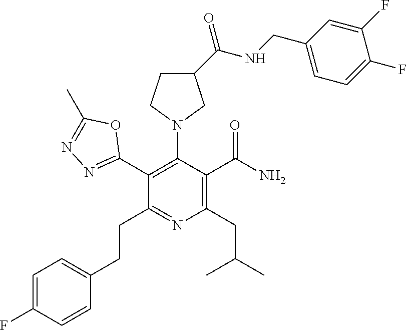 [SMILES: Cc1nnc(-c2c(CCc3ccc(F)cc3)nc(CC(C)C)c(C(N)=O)c2N2CCC(C(=O)NCc3ccc(F)c(F)c3)C2)o1]

To a solution of 5-(acetamidocarbamoyl)-4-[3-[(3,4-difluorophenyl)methylcarbamoyl]pyrrolidin-1-yl]-6-[2-(4-fluorophenyl)ethyl]-2-isobutyl-pyridine-3-carboxamide (30 mg, 46.97 μmol) in DCM (4 mL) was added 4-methylbenzenesulfonyl chloride (11 mg, 57.70 μmol) and TEA (36.35 mg, 359.23 μmol, 0.05 mL). The mixture was stirred at 22° C. for 16 hrs. The reaction mixture was adjusted pH to 5, and then diluted with water (30 mL) and extracted with EA (30 mL×2). The combined organic layers were washed with brine (30 mL), dried over Na₂SO₄, filtered and concentrated under reduced pressure to give a residue. The crude product was purified by prep-HPLC (Column: Kromasil 100-5-C18; Eluent: 25% to 65% water (0.1% FA)-ACN). 4-(3-((3,4-difluorobenzyl)carbamoyl)pyrrolidin-1-yl)-6-(4-fluorophenethyl)-2-isobutyl-5-(5-methyl-1,3,4-oxadiazol-2-yl)nicotinamide (11.48 mg, 18.24 μmol, 38.83% yield, 98.62% purity) was obtained.

¹H NMR (400 MHz, CDCl₃) δ ppm 7.03-7.16 (m, 2H), 6.84-7.00 (m, 5H), 6.53-6.70 (m, 2H), 6.10 (br s, 1H), 4.32 (qd, J=14.79, 5.75 Hz, 2H), 3.35-3.44 (m, 1H), 3.08-3.29 (m, 3H), 2.64-2.97 (m, 6H), 2.55 (s, 3H), 1.94-2.11 (m, 2H), 0.94 (t, J=6.48 Hz, 6H). LC-MS: m/z 621.3 (M+H)⁺.

### Example 33

### 4-(5-((3,4-difluorobenzyl)carbamoyl)thiophen-2-yl)-2-(4-fluorophenethyl)-6-isobutyl-6′-methyl-[3, 3′-bipyridine]-5-carboxamide (Compound 171)

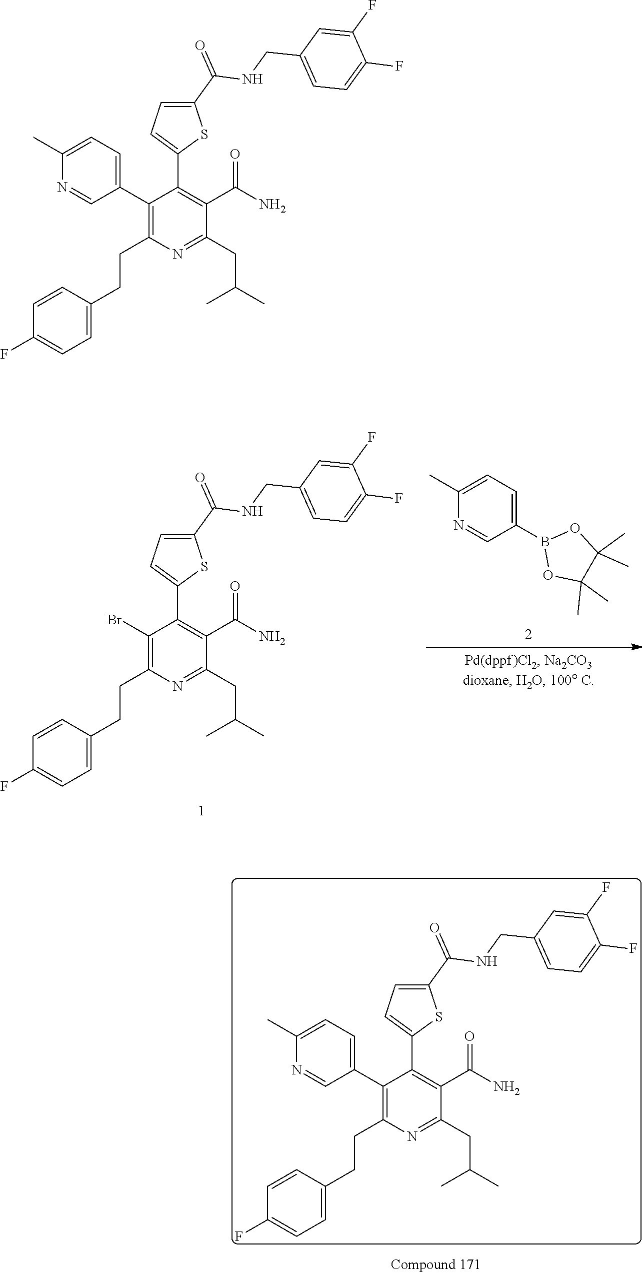

### Step A 4-(5-((3,4-difluorobenzyl)carbamoyl)thiophen-2-yl)-2-(4-fluorophenethyl)-6-isobutyl-6′-methyl-[3,3′-bipyridine]-5-carboxamide

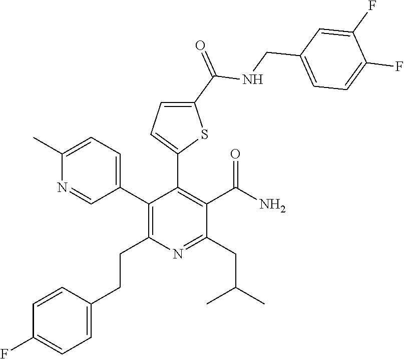 [SMILES: Cc1ccc(-c2c(CCc3ccc(F)cc3)nc(CC(C)C)c(C(N)=O)c2-c2ccc(C(=O)NCc3ccc(F)c(F)c3)s2)cn1]

A mixture of 5-bromo-4-(5-((3,4-difluorobenzyl)carbamoyl)thiophen-2-yl)-6-(4-fluorophenethyl)-2-isobutylnicotinamide (50 mg, 79.30 μmol), 2-methyl-5-(4,4,5,5-tetramethyl-1,3,2-dioxaborolan-2-yl)pyridine (17.37 mg, 79.30 μmol), Na₂CO₃ (16.81 mg, 158.60 μmol), Pd(dppf)Cl₂ (5.80 mg, 7.93 μmol) in dioxane (4 mL) and H₂O (1 mL) was degassed and purged with N₂ for 3 times, and then the mixture was stirred at 100° C. for 16 hrs under N₂ atmosphere. The reaction mixture was filtered and concentrated under reduced pressure to give a residue. The residue was purified by prep-HPLC (column: Phenomenex C18 80*40 mm*3 μm; mobile phase: [water (0.5% FA)-ACN]; gradient: 35%-75% B over 7 min) to give 4-(5-((3,4-difluorobenzyl)carbamoyl)thiophen-2-yl)-2-(4-fluorophenethyl)-6-isobutyl-6′-methyl-[3,3′-bipyridine]-5-carboxamide (14 mg, 21.67 μmol, 27.32% yield, 99.46% purity).

¹H NMR (400 MHz, CDCl₃) δ 7.97 (s, 1H), 7.24 (d, J=3.79 Hz, 1H), 7.06-7.14 (m, 2H), 7.03 (m, 3H), 6.82-6.94 (m, 5H), 6.45 (t, J=5.87 Hz, 1H), 5.80 (s, 1H), 5.67 (s, 1H), 4.43-4.48 (m, 2H), 2.91-2.98 (m, 2H), 2.77-2.89 (m, 4H), 2.51 (s, 3H), 2.34-2.44 (m, 1H), 1.00 (t, J=5.93 Hz, 6H). LC-MS: m/z 643.4 (M+H)⁺.

### 4-(5-((3,4-difluorobenzyl)carbamoyl)thiophen-2-yl)-6-(4-fluorophenethyl)-2-isobutyl-5-(1-methyl-1H-pyrazol-3-yl)nicotinamide (Compound 204)

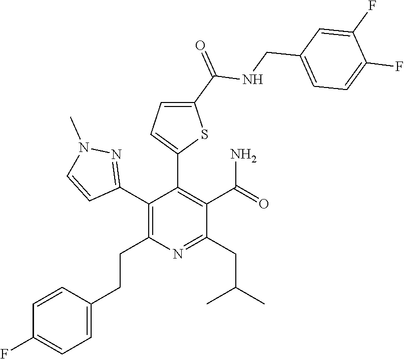 [SMILES: CC(C)Cc1nc(CCc2ccc(F)cc2)c(-c2ccn(C)n2)c(-c2ccc(C(=O)NCc3ccc(F)c(F)c3)s2)c1C(N)=O]

Compound 204 was synthesized using a similar procedure described in the Example 33 above by using the appropriate materials.

¹H NMR (400 MHz, CDCl₃) δ 7.31-7.35 (m, 1H), 7.28 (d, J=2.08 Hz, 1H), 7.08-7.18 (m, 2H), 6.98-7.07 (m, 4H), 6.86-6.94 (m, 2H), 6.55-6.62 (m, 1H), 5.72-5.92 (m, 3H), 4.48-4.53 (m, 2H), 3.91 (s, 3H), 3.03-3.11 (m, 2H), 2.91-2.98 (m, 2H), 2.85-2.90 (m, 2H), 2.23-2.35 (m, 1H), 0.97 (s, 3H), 0.95 (s, 3H). LC-MS: m/z 632.3 (M+H)⁺.

### Example 34

### 4-(5-((3,4-difluorobenzyl)carbamoyl)thiophen-2-yl)-6-((1-(4-fluorophenyl)cyclopropyl)methyl)-2-isobutyl-5-(5-methyl-1,3,4-oxadiazol-2-yl)nicotinamide (Compound 172)

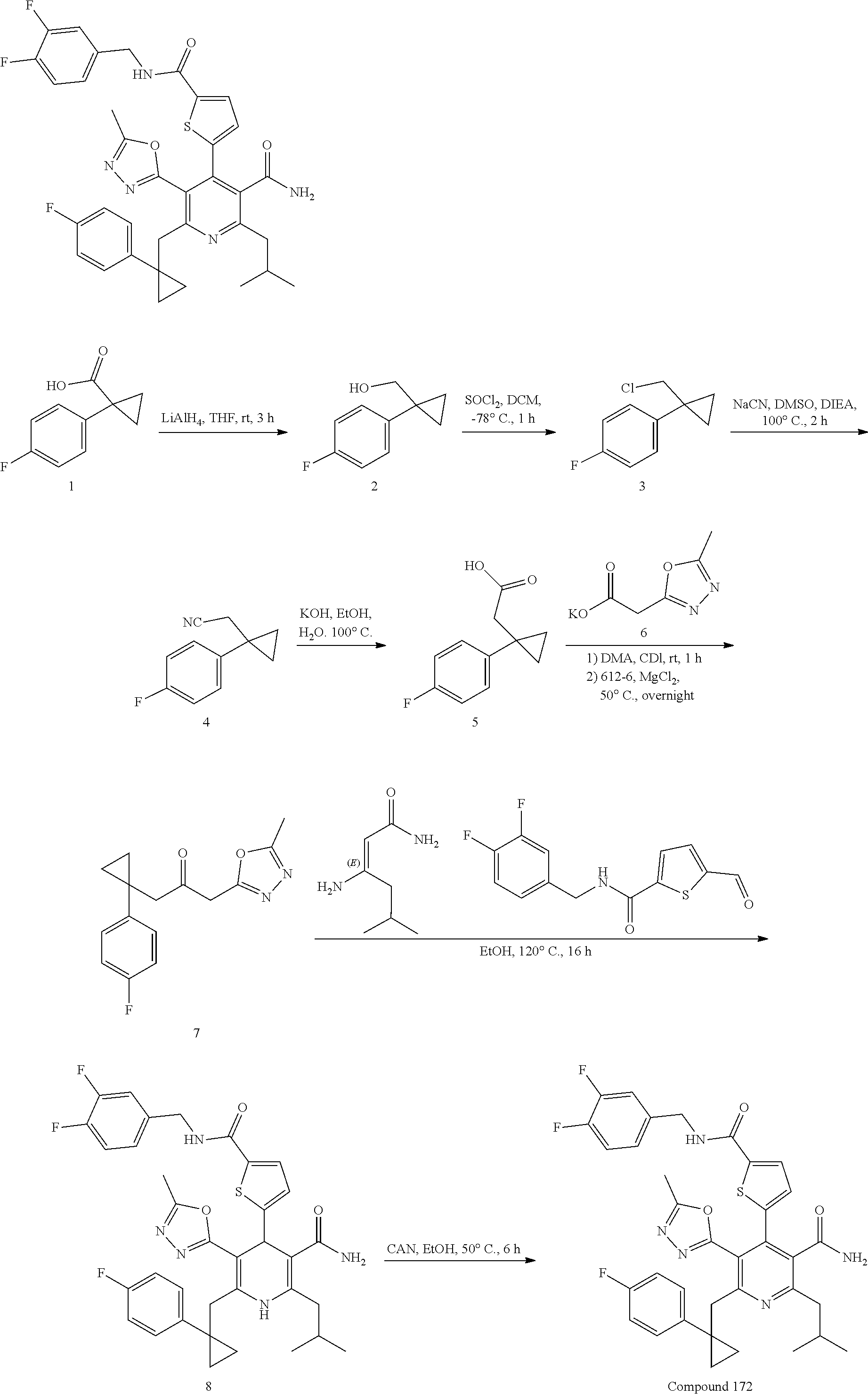

### Step A (1-(4-fluorophenyl)cyclopropyl)methanol

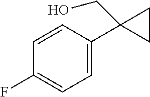 [SMILES: OCC1(c2ccc(F)cc2)CC1]

To a solution of 1-(4-fluorophenyl)cyclopropane-1-carboxylic acid (9.5 g, 52.725 mmol) in THF (100 mL) was added LiAlH₄ (3.00 g, 79.088 mmol) at 0° C., and the reaction was stirred at room temperature for 3 hr. After the reaction was completed, the mixture was quenched with water (100 mL), diluted with EA (200 mL), washed with water (600 mL). The organic layers were dried over Na₂SO₄, filtered and concentrated under vacuum to dryness. The residue was purified by column chromatography on silica gel (PE/EA=5/1) to give (1-(4-fluorophenyl)cyclopropyl)methanol (8.4 g, 50.542 mmol, 95.86%).

¹H NMR (400 MHz, DMSO-d6): δ 7.31-7.34 (m, 2H), 7.05-7.10 (m, 2H), 4.68 (t, J=5.6 Hz, 1H), 3.49 (d, J=5.6 Hz, 2H), 0.80-0.83 (m, 2H), 0.68-0.71 (m, 2H).

### Step B 1-(1-(chloromethyl)cyclopropyl)-4-fluorobenzene

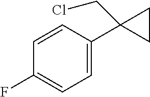 [SMILES: Fc1ccc(C2(CCl)CC2)cc1]

To a solution of (1-(4-fluorophenyl)cyclopropyl)methanol (7 g, 42.118 mmol) in DCM (100 mL) was slowed added SOCl2 (6.118 mL, 84.236 mmol) at −78° C., and the reaction was stirred at−78° C. for 1 h. After the reaction was completed, the mixture was diluted with DCM (50 mL), adjusted pH to 8 with NaHCO₃ saturated aqueous solution, washed with water (500 mL). The organic layers were dried over Na2SO4, filtered and concentrated to give 1-(1-(chloromethyl)cyclopropyl)-4-fluorobenzene (7.5 g, 32.496 mmol, 77.15%).

1H NMR (400 MHz, DMSO-d6): δ 7.29-7.33 (m, 2H), 7.11 (t, J=8.8 Hz, 2H), 3.88 (s, 2H), 0.86 (s, 4H).

### Step C 2-(1-(4-fluorophenyl)cyclopropyl)acetonitrile

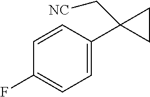 [SMILES: N#CCC1(c2ccc(F)cc2)CC1]

To a solution of 1-(1-(chloromethyl)cyclopropyl)-4-fluorobenzene (5.5 g, 29.788 mmol) in DMSO (1 mL) were added sodium cyanide (19.91 mg, 0.406 mmol), DIEA (15.566 mL, 89.363 mmol), and the reaction was stirred at 100° C. for 2 h. After the reaction was completed, the mixture was diluted with EA (70 mL), washed with Na₂CO₃ saturated solution (500 mL) and water (500 mL). The organic layers were dried over Na₂SO₄, filtered and concentrated under vacuum to dryness. The residue was purified by column chromatography on silica gel (PE/EA=20/1) to give 2-(1-(4-fluorophenyl)cyclopropyl)acetonitrile (2.6 g, 14.839 mmol, 49.82%).

1H NMR (400 MHz, DMSO-d6): δ 7.37-7.41 (m, 2H), 7.13-7.21 (m, 2H), 2.89 (s, 2H), 0.89-0.96 (m, 4H).

### Step D 2-(1-(4-fluorophenyl)cyclopropyl)acetic acid

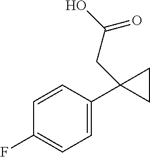 [SMILES: O=C(O)CC1(c2ccc(F)cc2)CC1]

A mixture of 2-(1-(4-fluorophenyl)cyclopropyl)acetonitrile (1 g, 5.707 mmol) and KOH (4.80 g, 85.612 mmol) in EtOH (15 mL) and H2O (5 mL) was stirred at 100° C. for overnight. After the reaction was completed, the mixture was diluted with EA (30 mL), adjusted pH to 6 with 1 N aq. HCl, washed with water (100 mL). The organic layers were dried over Na2SO4, filtered and concentrated under vacuum to dryness. The residue was purified by column chromatography on C18 (FA 0.10%/ACN=50-55%) to give 2-(1-(4-fluorophenyl)cyclopropyl)acetic acid (1 g, 5.149 mmol, 90.22%). LC-MS: m/z 192.9 (M−H)⁻.

### Step E 1-(1-(4-fluorophenyl)cyclopropyl)-3-(5-methyl-1,3,4-oxadiazol-2-yl)propan-2-one

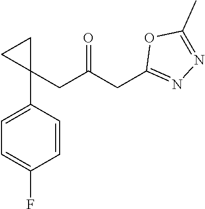 [SMILES: Cc1nnc(CC(=O)CC2(c3ccc(F)cc3)CC2)o1]

To a solution of 2-(1-(4-fluorophenyl)cyclopropyl)acetic acid (900 mg, 4.634 mmol) in DMA (10 mL) was added CDI (1127.14 mg, 6.951 mmol), and the reaction was stirred at room temperature for 1 hr. Then, potassium 2-(5-methyl-1,3,4-oxadiazol-2-yl)acetate (1252.61 mg, 6.951 mmol) and MgCl2 (441.22 mg, 4.634 mmol) were added, the reaction was stirred at 50° C. for overnight. After the reaction was completed, the mixture was diluted with EA (30 mL), washed with water (100 mL). The organic layers were dried over Na2SO4, filtered and concentrated under vacuum to dryness. The residue was purified by column chromatography on silica gel (PE/EA=1/1) to give 1-(1-(4-fluorophenyl)cyclopropyl)-3-(5-methyl-1,3,4-oxadiazol-2-yl)propan-2-one (800 mg, 2.917 mmol, 62.94%). LC-MS: m/z 275.1 (M+H)⁺.

### Step F 4-(5-((3,4-difluorobenzyl)carbamoyl)thiophen-2-yl)-6-((1-(4-fluorophenyl)cyclopropyl)methyl)-2-isobutyl-5-(5-methyl-1,3,4-oxadiazol-2-yl)-1,4-dihydropyridine-3-carboxamide

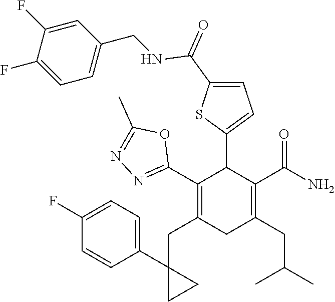 [SMILES: Cc1nnc(C2=C(CC3(c4ccc(F)cc4)CC3)CC(CC(C)C)=C(C(N)=O)C2c2ccc(C(=O)NCc3ccc(F)c(F)c3)s2)o1]

A 1-(1-(4-fluorophenyl)cyclopropyl)-3-(5-methyl-1,3,4-oxadiazol-2-yl)propan-2-one (50 mg, 0.182 mmol), (E)-3-amino-5-methylhex-2-enamide (25.92 mg, 0.182 mmol) and N-(3,4-difluorobenzyl)-5-formylthiophene-2-carboxamide (51.27 mg, 0.182 mmol) in EtOH (2 mL) in 10 mL of sealed tube. The reaction was stirred at 120° C. for 16 h. After the reaction was completed, the mixture was concentrated to give 4-(5-((3,4-difluorobenzyl)carbamoyl)thiophen-2-yl)-6-((1-(4-fluorophenyl)cyclopropyl)methyl)-2-isobutyl-5-(5-methyl-1,3,4-oxadiazol-2-yl)-1,4-dihydropyridine-3-carboxamide (120 mg, crude). LC-MS: m/z 662.1 (M+H)⁺.

### Step G 4-(5-((3,4-difluorobenzyl)carbamoyl)thiophen-2-yl)-6-((1-(4-fluorophenyl)cyclopropyl)methyl)-2-isobutyl-5-(5-methyl-1,3,4-oxadiazol-2-yl)nicotinamide

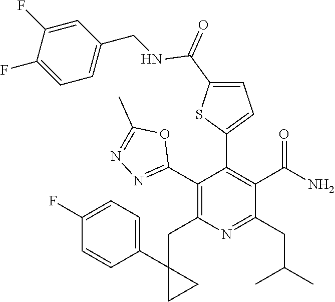 [SMILES: Cc1nnc(-c2c(CC3(c4ccc(F)cc4)CC3)nc(CC(C)C)c(C(N)=O)c2-c2ccc(C(=O)NCc3ccc(F)c(F)c3)s2)o1]

To a solution of 4-(5-((3,4-difluorobenzyl)carbamoyl)thiophen-2-yl)-6-((1-(4-fluorophenyl)cyclopropyl)methyl)-2-isobutyl-5-(5-methyl-1,3,4-oxadiazol-2-yl)-1,4-dihydropyridine-3-carboxamide (120 mg, 0.181 mmol) in EtOH (3 mL) was added ceric ammonium nitrate (99.41 mg, 0.181 mmol), and the reaction was stirred at 50° C. for 6 hr. After the reaction was completed, the mixture was diluted with EA (20 mL), washed with water (100 mL). The organic layers were dried over Na₂SO₄, filtered and concentrated under vacuum to dryness. The residue was purified by Prep-HPLC to give 4-(5-((3,4-difluorobenzyl)carbamoyl)thiophen-2-yl)-6-((1-(4-fluorophenyl)cyclopropyl)methyl)-2-isobutyl-5-(5-methyl-1,3,4-oxadiazol-2-yl)nicotinamide (19.94 mg, 0.030 mmol, 16.67%).

1H NMR (400 MHz, DMSO-d6): δ 9.10 (t, J=5.6 Hz, 1H), 7.83 (s, 1H), 7.61 (d, J=4.0 Hz, 1H), 7.54 (s, 1H), 7.28-7.44 (m, 2H), 7.09-7.17 (m, 1H), 6.96 (d, J=7.6 Hz, 4H), 6.89 (d, J=4.0 Hz, 1H), 4.39 (d, J=6.0 Hz, 2H), 3.12 (s, 2H), 2.68 (d, J=7.2 Hz, 2H), 2.19-2.32 (m, 4H), 0.83-0.97 (m, 8H), 0.64-0.76 (m, 2H). ¹⁹F NMR (377 MHz, DMSO-d6): −116.77, −138.89, −141.38. LC-MS: m/z 660.2 (M+H)⁺.

### 4-(5-((3,4-difluorobenzyl)carbamoyl)thiophen-2-yl)-6-(2-(4-fluorophenyl)-2-methylpropyl)-2-isobutyl-5-(5-methyl-1,3,4-oxadiazol-2-yl)nicotinamide (Compound 176)

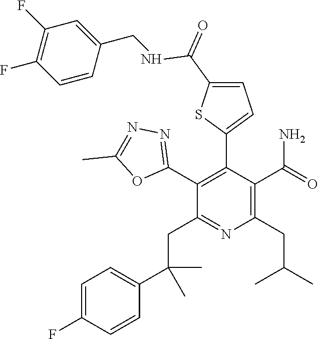 [SMILES: Cc1nnc(-c2c(CC(C)(C)c3ccc(F)cc3)nc(CC(C)C)c(C(N)=O)c2-c2ccc(C(=O)NCc3ccc(F)c(F)c3)s2)o1]

Compound 176 was synthesized using a similar procedure described in the Example 34 above by using the appropriate materials.

¹H NMR (400 MHz, Methanol-d4): δ7.51 (d, J=3.6 Hz, 1H), 7.17-7.23 (m, 2H), 7.09-7.12 (m, 1H), 7.01-7.05 (m, 2H), 6.92 (d, J=4.0 Hz, 1H), 6.85 (t, J=8.8 Hz, 2H), 4.45 (s, 2H), 3.30 (s, 2H), 2.76 (d, J=7.2 Hz, 2H), 2.31 (p, J=6.8 Hz, 1H), 2.26 (s, 3H), 1.36 (s, 6H), 0.97 (d, J=6.4 Hz, 6H). ¹⁹F NMR (377 MHz, Methanol-d4): −119.91. −140.57, −142.99. LC-MS: m/z 662.2 (M+H)⁺.

### 4-(5-((3,4-difluorobenzyl)carbamoyl)thiophen-2-yl)-6-(2-(4-fluorophenyl)cyclopropyl)-2-isobutyl-5-(5-methyl-1,3,4-oxadiazol-2-yl)nicotinamide (Compound 178)

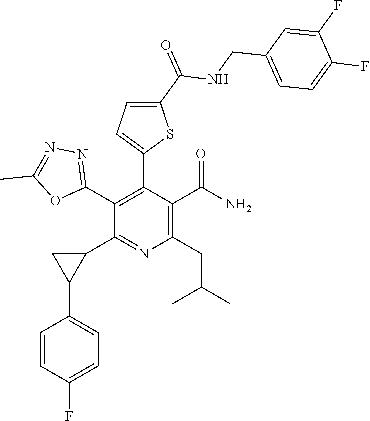 [SMILES: Cc1nnc(-c2c(C3CC3c3ccc(F)cc3)nc(CC(C)C)c(C(N)=O)c2-c2ccc(C(=O)NCc3ccc(F)c(F)c3)s2)o1]

Compound 178 was synthesized using a similar procedure described in the Example 34 above by using the appropriate materials.

¹H NMR (400 MHz, MeOD-d₄) δ 8.51 (br s, 1H), 7.58 (d, J=4.00 Hz, 1H), 7.21-7.28 (m, 2H), 7.11-7.17 (m, 3H), 7.08 (d, J=4.00 Hz, 1H), 6.97-7.03 (m, 2H), 4.49 (s, 2H), 2.84 (d, J=7.20 Hz, 2H), 2.53-2.59 (m, 1H), 2.38-2.45 (m, 1H), 2.36 (s, 3H), 2.12-2.22 (m, 1H), 1.91-2.01 (m, 1H), 1.43-1.55 (m, 1H), 1.04 (d, J=6.60, 1.60 Hz, 3H), 1.02 (d, J=6.60, 1.60 Hz, 3H). LC-MS: m/z 646.4 (M+H)⁺.

### 4-(5-((3,4-difluorobenzyl)carbamoyl)thiophen-2-yl)-6-((2,3-dihydrobenzo[b][1,4]dioxin-2-yl)methyl)-2-isobutyl-5-(5-methyl-1,3,4-oxadiazol-2-yl)nicotinamide (Compound 180)

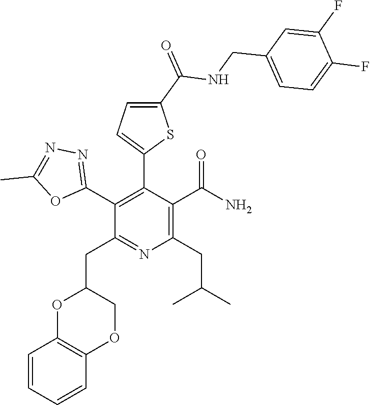 [SMILES: Cc1nnc(-c2c(CC3COc4ccccc4O3)nc(CC(C)C)c(C(N)=O)c2-c2ccc(C(=O)NCc3ccc(F)c(F)c3)s2)o1]

Compound 180 was synthesized using a similar procedure described in the Example 34 above by using the appropriate materials.

¹H NMR (400 MHz, CDCl₃) δ ppm 7.34 (d, J=3.79 Hz, 1H), 7.04-7.16 (m, 3H), 6.95-7.01 (m, 2H), 6.76-6.85 (m, 3H), 6.64-6.73 (m, 1H), 6.14 (br s, 1H), 6.03 (s, 1H), 4.66-4.75 (m, 1H), 4.43 (d, J=5.87 Hz, 2H), 4.32 (dd, J=11.37, 1.96 Hz, 1H), 3.94 (dd, J=11.37, 6.60 Hz, 1H), 3.23 (dd, J=14.61, 7.27 Hz, 1H), 3.07 (dd, J=14.55, 5.75 Hz, 1H), 2.82 (d, J=6.85 Hz, 2H), 2.28-2.42 (m, 4H), 0.94-1.00 (m, 1H), 0.98 (t, J=6.05 Hz, 6H). LC-MS: m/z 660.4 (M+H)⁺.

### 4-(5-((3,4-difluorobenzyl)carbamoyl)thiophen-2-yl)-6-(4-fluorobenzyl)-2-isobutyl-5-(5-methyl-1,3,4-oxadiazol-2-yl)nicotinamide (Compound 181)

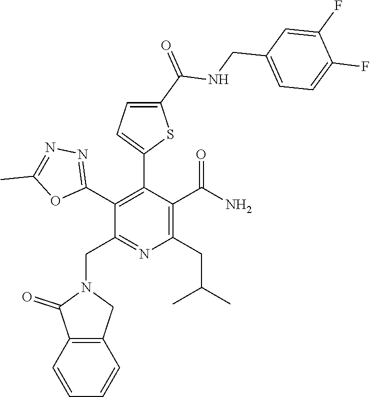 [SMILES: Cc1nnc(-c2c(CN3Cc4ccccc4C3=O)nc(CC(C)C)c(C(N)=O)c2-c2ccc(C(=O)NCc3ccc(F)c(F)c3)s2)o1]

Compound 181 was synthesized using a similar procedure described in the Example 34 above by using the appropriate materials.

¹H NMR (400 MHz, DMSO-d₆) δ 9.18 (br t, J=5.99 Hz, 1H), 7.96 (br s, 1H), 7.56-7.70 (m, 5H), 7.45-7.52 (m, 1H), 7.29-7.41 (m, 2H), 7.13 (br s, 1H), 6.99 (d, J=3.79 Hz, 1H), 4.85 (s, 2H), 4.47 (s, 2H), 4.38 (br d, J=5.75 Hz, 2H), 2.61 (br d, J=7.09 Hz, 2H), 2.33-2.38 (m, 3H), 1.98-2.06 (m, 1H), 0.74 (d, J=6.60 Hz, 6H)¹H NMR (400 MHz, CHLOROFORM-d) δ 7.34 (d, J=3.91 Hz, 1H), 7.08-7.19 (m, 2H), 6.97-7.07 (m, 4H), 6.85-6.93 (m, 2H), 6.37 (br t, J=5.87 Hz, 1H), 5.51-5.70 (m, 2H), 4.51 (d, J=5.87 Hz, 2H), 4.20 (s, 2H), 2.86 (d, J=7.21 Hz, 2H), 2.35-2.44 (m, 1H), 2.32 (s, 3H), 0.99 (d, J=6.60 Hz, 6H). LC-MS: m/z 657.1 (M+H)⁺.

### 4-(5-((3,4-difluorobenzyl)carbamoyl)thiophen-2-yl)-2-isobutyl-5-(5-methyl-1,3,4-oxadiazol-2-yl)-6-(2-(3-methyl-1H-pyrazol-1-yl)ethyl)nicotinamide (Compound 183)

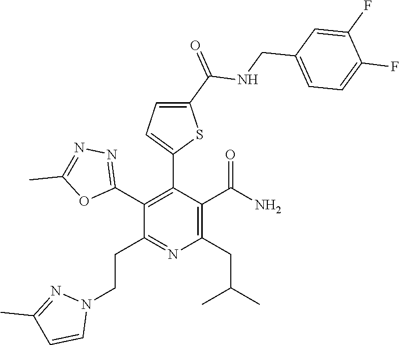 [SMILES: Cc1ccn(CCc2nc(CC(C)C)c(C(N)=O)c(-c3ccc(C(=O)NCc4ccc(F)c(F)c4)s3)c2-c2nnc(C)o2)n1]

Compound 183 was synthesized using a similar procedure described in the Example 34 above by using the appropriate materials.

¹H NMR (400 MHz, MeOD-d₄) δ 7.55 (d, J=3.91 Hz, 1H), 7.16-7.31 (m, 3H), 7.09-7.15 (m, 1H), 7.02 (d, J=3.79 Hz, 1H), 5.92-5.98 (m, 1H), 4.48-4.56 (m, 2H), 4.45-4.48 (m, 2H), 3.31-3.36 (m, 2H), 2.83 (dd, J=2.32, 7.21 Hz, 2H), 2.41 (d, J=12.59 Hz, 4H), 2.16 (d, J=4.65 Hz, 3H), 1.00 (d, J=6.72 Hz, 6H), 0.94-1.03 (m, 1H). LC-MS: m/z 620.4 (M+H)⁺.

### 4-(5-((3,4-difluorobenzyl)carbamoyl)thiophen-2-yl)-2-isobutyl-5-(5-methyl-1,3,4-oxadiazol-2-yl)-6-(2-(1-methyl-1H-pyrazol-4-yl)ethyl)nicotinamide (Compound 184)

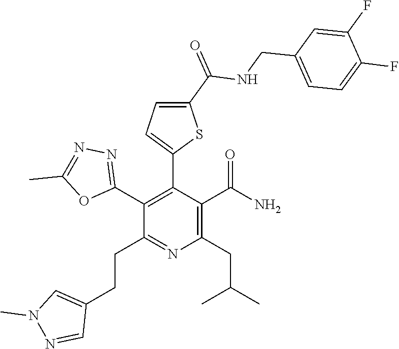 [SMILES: Cc1nnc(-c2c(CCc3cnn(C)c3)nc(CC(C)C)c(C(N)=O)c2-c2ccc(C(=O)NCc3ccc(F)c(F)c3)s2)o1]

Compound 184 was synthesized using a similar procedure described in the Example 34 above by using the appropriate materials.

¹H NMR (400 MHz, CDCl₃) δ ppm 7.35 (d, J=3.79 Hz, 1H), 7.08-7.18 (m, 3H), 7.05 (s, 2H), 6.99 (d, J=3.79 Hz, 1H), 6.57-6.67 (m, 1H), 5.75 (d, J=11.13 Hz, 2H), 4.50 (d, J=5.99 Hz, 2H), 3.81 (s, 3H), 2.96-3.02 (m, 2H), 2.83-2.91 (m, 4H), 2.37-2.44 (m, 4H), 0.99 (d, J=6.60 Hz, 6H). LC-MS: m/z 620.4 (M+H)⁺.

### 4-(5-((3,4-difluorobenzyl)carbamoyl)thiophen-2-yl)-6-(2-(4-fluorophenyl)-2-methoxyethyl)-2-isobutyl-5-(5-methyl-1,3,4-oxadiazol-2-yl)nicotinamide (Compound 186)

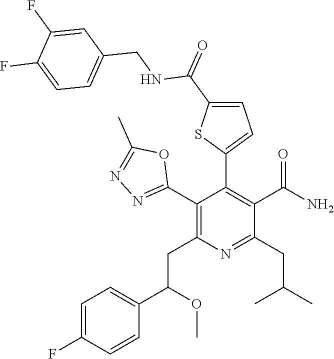 [SMILES: COC(Cc1nc(CC(C)C)c(C(N)=O)c(-c2ccc(C(=O)NCc3ccc(F)c(F)c3)s2)c1-c1nnc(C)o1)c1ccc(F)cc1]

Compound 186 was synthesized using a similar procedure described in the Example 34 above by using the appropriate materials.

¹H NMR (400 MHz, DMSO-d6): δ 9.13 (t, J=6.0 Hz, 1H), 7.89 (br s, 1H), 7.64 (d, J=3.6 Hz, 1H), 7.58 (br s, 1H), 7.31-7.43 (m, 2H), 7.10-7.20 (m, 5H), 6.95 (d, J=3.6 Hz, 1H), 4.68 (t, J=6.4 Hz, 1H), 4.40 (d, J=6.0 Hz, 2H), 3.20-3.25 (m, 1H), 3.02-3.07 (m, 1H), 3.00 (s, 3H), 2.69-2.75 (m, 2H), 2.37 (s, 3H), 2.25-2.32 (m, 1H), 0.93 (dd, J=6.4 Hz, J=2.8 Hz, 6H). ¹⁹F NMR (377 MHz, DMSO-d6): −114.82, −138.45, 141.34. LC-MS: m/z 664.1 (M+H)⁺.

### (R)-6-ethyl-4-(7-((5-fluoro-2,3-dihydro-1H-inden-1-yl)amino)thieno[2,3-c]pyridin-2-yl)-2-isobutyl-5-(5-methyl-1,3,4-oxadiazol-2-yl)nicotinamide (Compound 188)

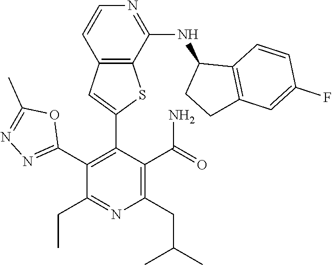 [SMILES: CCc1nc(CC(C)C)c(C(N)=O)c(-c2cc3ccnc(N[C@@H]4CCc5cc(F)ccc54)c3s2)c1-c1nnc(C)o1]

Compound 188 was synthesized using a similar procedure described in the Example 34 above by using the appropriate materials.

¹H NMR (400 MHz, DMSO-d₆): δ 7.91-7.94 (m, 2H), 7.59 (br s, 1H), 7.26 (s, 1H), 7.17-7.24 (m, 2H), 7.06-7.11 (m, 1H), 7.04 (d, J=5.6 Hz, 1H), 6.90-6.97 (m, 1H), 5.81 (q, J=8.0 Hz, 1H), 2.97-3.03 (m, 1H), 2.83-2.88 (m, 1H), 2.75 (d, J=7.2 Hz, 2H), 2.64-2.70 (m, 3H), 2.38 (s, 3H), 2.28-2.33 (m, 1H), 2.00-2.10 (m, 1H), 1.19 (t, J=7.2 Hz, 3H), 0.96 (d, J=6.8 Hz, 6H). ¹⁹F NMR (377 MHz, DMSO-d₆):−116.34. LC-MS: m/z 571.3 (M+H)⁺.

### 4-(5-((3,4-difluorobenzyl)carbamoyl)thiophen-2-yl)-6-(3,3-difluoropropyl)-2-isobutyl-5-(5-methyl-1,3,4-oxadiazol-2-yl)nicotinamide (Compound 189)

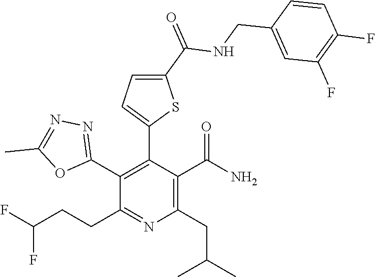 [SMILES: Cc1nnc(-c2c(CCC(F)F)nc(CC(C)C)c(C(N)=O)c2-c2ccc(C(=O)NCc3ccc(F)c(F)c3)s2)o1]

Compound 189 was synthesized using a similar procedure described in the Example 34 above by using the appropriate materials.

¹H NMR (400 MHz, DMSO-d6): δ 9.14 (t, J=6.0 Hz, 1H), 7.90 (s, 1H), 7.66 (d, J=4.0 Hz, 1H), 7.62 (s, 1H), 7.30-7.45 (m, 2H), 7.11-7.18 (m, 1H), 6.99 (d, J=3.6 Hz, 1H), 5.99-6.34 (m, 1H), 4.41 (d, J=6.0 Hz, 1H), 2.78-2.86 (m, 2H), 2.74 (d, J=7.2 Hz, 1H), 2.43 (s, 3H), 2.22-2.33 (m, 3H), 0.95 (d, J=6.8 Hz, 6H). ¹⁹F NMR (377 MHz, DMSO-d6): −115.94, −138.87, −141.36. LC-MS: m/z 590.2 (M+H)⁺.

### 4-(5-((3,4-difluorobenzyl)carbamoyl)thiophen-2-yl)-6-(3-(difluoromethyl)bicyclo[1.1.1]pentan-1-yl)-2-isobutyl-5-(5-methyl-1,3,4-oxadiazol-2-yl)nicotinamide (Compound 190)

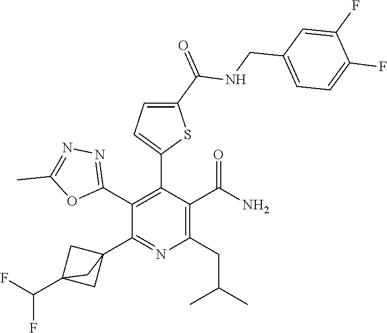 [SMILES: Cc1nnc(-c2c(C34CC(C(F)F)(C3)C4)nc(CC(C)C)c(C(N)=O)c2-c2ccc(C(=O)NCc3ccc(F)c(F)c3)s2)o1]

Compound 190 was synthesized using a similar procedure described in the Example 34 above by using the appropriate materials.

¹H NMR (400 MHz, CDCl₃) δ 7.29-7.33 (m, 1H), 7.07-7.17 (m, 2H), 6.94-7.05 (m, 2H), 6.68-6.79 (m, 1H), 5.49-5.90 (m, 3H), 4.44-4.52 (m, 2H), 2.77-2.86 (m, 2H), 2.50-2.56 (m, 3H), 2.28-2.42 (m, 1H), 1.96-2.04 (m, 6H), 0.99 (s, 3H), 0.97 (s, 3H). LC-MS: m/z 628.4 (M+H)⁺.

### 4-(5-(((R)-6,7-dihydro-5H-cyclopenta[b]pyridin-5-yl)carbamoyl)thiophen-2-yl)-6-(2-(4-fluorophenyl)propyl)-2-isobutyl-5-(5-methyl-1,3,4-oxadiazol-2-yl)nicotinamide (Compound 191)

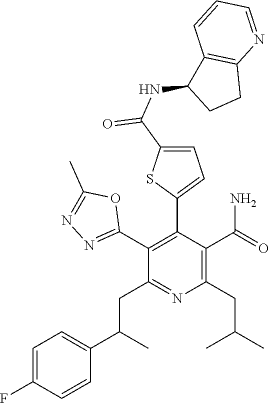 [SMILES: Cc1nnc(-c2c(CC(C)c3ccc(F)cc3)nc(CC(C)C)c(C(N)=O)c2-c2ccc(C(=O)N[C@@H]3CCc4ncccc43)s2)o1]

Compound 191 was synthesized using a similar procedure described in the Example 34 above by using the appropriate materials.

¹H NMR (400 MHz, DMSO-d₆): δ 8.91 (d, J=8.0 Hz, 1H), 8.40 (d, J=4.4 Hz, 1H), 7.87 (s, 1H), 7.51-7.70 (m, 3H), 7.19 (dd, J=7.6 Hz, J=5.2 Hz, 1H), 6.97-7.13 (m, 4H), 6.92 (d, J=3.6 Hz, 1H), 5.48 (dd, J=16.0 Hz, J=8.0 Hz, 1H), 3.23-3.29 (m, 1H), 2.87-3.08 (m, 4H), 2.70 (d, J=7.2 Hz, 2H), 2.45-2.48 (m, 1H), 2.40 (s, 3H), 2.22-2.32 (m, 1H), 1.93-2.03 (m, 1H), 1.19 (d, J=6.8 Hz, 3H), 0.91 (t, J=6.0 Hz, 6H). ¹⁹F NMR (377 MHz, DMSO-d₆): −117.15. LC-MS: m/z 639.3 (M+H)⁺.

### 4-(5-((3,4-difluorobenzyl)carbamoyl)thiophen-2-yl)-6-((2,2-difluorocyclopropyl)methyl)-2-isobutyl-5-(5-methyl-1,3,4-oxadiazol-2-yl)nicotinamide (Compound 192)

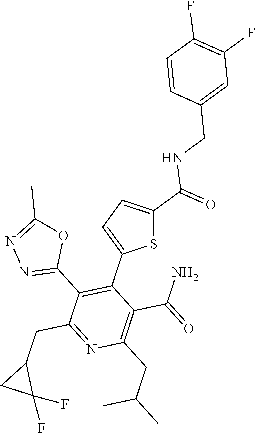

Compound 192 was synthesized using a similar procedure described in the Example 34 above by using the appropriate materials.

¹H NMR (400 MHz, Methanol-d₄): δ7.56 (d, J=4.0 Hz, 1H), 7.17-7.24 (m, 2H), 7.10-7.14 (m, 1H), 7.05 (d, J=4.0 Hz, 1H), 4.46 (s, 2H), 2.92-2.96 (m, 2H), 2.85 (d, J=7.2 Hz, 2H), 2.43 (S, 3H), 2.39-2.43 (m, 1H), 2.01-2.10 (m, 1H), 1.40-1.48 (m, 1H), 1.11-1.18 (m, 1H), 1.01 (dd, J1=1.6 Hz, J2=5.2 Hz, 6H). ¹⁹F NMR (377 MHz, Methanol-d₄): −130.22, −130.63, −140.55, −140.60, −142.97, −143.02, −144.97, −145.38. LC-MS: m/z 602.2 (M+H)⁺.

### 4-(5-((3,4-difluorobenzyl)carbamoyl)thiophen-2-yl)-6-((3,3-difluorocyclobutyl)methyl)-2-isobutyl-5-(5-methyl-1,3,4-oxadiazol-2-yl)nicotinamide (Compound 193)

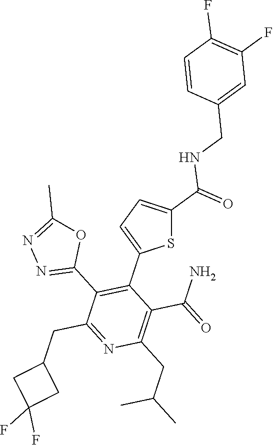

Compound 193 was synthesized using a similar procedure described in the Example 34 above by using the appropriate materials.

¹H NMR (400 MHz, Methanol-d₄): δ7.55 (d, J=4.0 Hz, 1H), 7.17-7.24 (m, 2H), 7.10-7.14 (m, 1H), 7.04 (d, J=4.0 Hz, 1H), 4.46 (s, 2H), 2.94 (d, J=6.8 Hz, 2H), 2.83 (d, J=7.2 Hz, 2H), 2.60-2.66 (m, 2H), 2.58-2.59 (m, 1H), 2.46 (s, 3H), 2.33-2.41 (m, 1H), 2.27-2.31 (m, 2H), 1.00 (d, J=6.4 Hz, 6H). ¹⁹F NMR (377 MHz, Methanol-d₄): −84.27, −84.78, −96.36, −96.88, −140.54, −140.59, −142.96, −143.01. LC-MS: m/z 616.2 (M+H)⁺.

### 4-(5-((3,4-difluorobenzyl)carbamoyl)thiophen-2-yl)-2-isobutyl-5-(5-methyl-1,3,4-oxadiazol-2-yl)-6-(3-(trifluoromethyl)bicyclo[1.1.1]pentan-1-yl)nicotinamide (Compound 194)

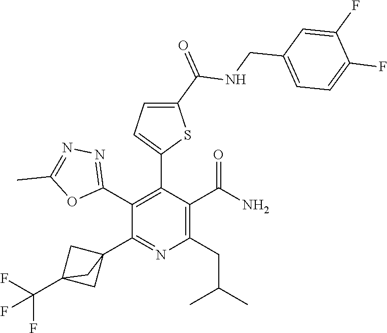 [SMILES: Cc1nnc(-c2c(C34CC(C(F)(F)F)(C3)C4)nc(CC(C)C)c(C(N)=O)c2-c2ccc(C(=O)NCc3ccc(F)c(F)c3)s2)o1]

Compound 194 was synthesized using a similar procedure described in the Example 34 above by using the appropriate materials.

¹H NMR (400 MHz, CDCl₃) δ 7.33 (d, J=3.60 Hz, 1H), 7.08-7.19 (m, 2H), 6.98-7.05 (m, 1H), 7.02 (d, J=3.60 Hz, 1H), 6.54 (t, J=6.00 Hz, 1H), 5.74 (s, 1H), 5.56 (s, 1H), 4.50 (d, J=6.00 Hz, 2H), 2.82 (d, J=7.20 Hz, 2H), 2.53 (s, 3H), 2.29-2.41 (m, 1H), 2.11 (s, 6H), 0.99 (s, 3H), 0.97 (s, 3H). LC-MS: m/z 646.3 (M+H)⁺.

### (R)-4-(5-((6,7-dihydro-5H-cyclopenta[b]pyridin-5-yl)carbamoyl)thiophen-2-yl)-2-isobutyl-5-(5-methyl-1,3,4-oxadiazol-2-yl)-6-(2-(5-methylpyridin-2-yl)ethyl)nicotinamide (Compound 195)

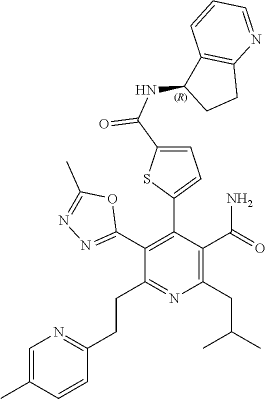

Compound 195 was synthesized using a similar procedure described in the Example 34 above by using the appropriate materials.

¹H NMR (400 MHz, Methanol-d₄): δ 8.37 (d, J=5.2 Hz, 1H), 8.21 (s, 1H), 7.73 (d, J=7.6 Hz, 1H), 7.56 (d, J=3.6 Hz, 1H), 7.51 (d, J=8.0 Hz, 1H), 7.24-7.27 (m, 1H), 7.06 (d, J=8.0 Hz, 1H), 7.01 (d, J=3.6 Hz, 1H), 5.60 (t, J=8.0 Hz, 1H), 3.19 (s, 4H), 3.11-3.14 (m, 1H), 2.99-3.05 (m, 1H), 2.81 (d, J=7.2 Hz, 2H), 2.62-2.66 (m, 1H), 2.45 (s, 3H), 3.31-3.33 (m, 1H), 2.29 (s, 3H), 2.05-2.10 (m, 1H), 0.97 (d, J=6.8 Hz, 6H). LC-MS: m/z 622.2 (M+H)⁺.

### 4-(5-((3,4-difluorobenzyl)carbamoyl)thiophen-2-yl)-6-((3-(difluoromethyl)bicyclo[1.1.1]pentan-1-yl)methyl)-2-isobutyl-5-(5-methyl-1,3,4-oxadiazol-2-yl)nicotinamide (Compound 203)

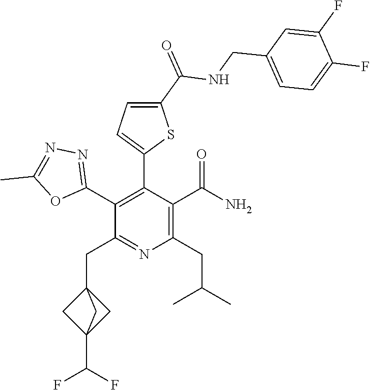 [SMILES: Cc1nnc(-c2c(CC34CC(C(F)F)(C3)C4)nc(CC(C)C)c(C(N)=O)c2-c2ccc(C(=O)NCc3ccc(F)c(F)c3)s2)o1]

Compound 203 was synthesized using a similar procedure described in the Example 34 above by using the appropriate materials.

¹H NMR (400 MHz, CDCl₃) δ 7.32-7.38 (m, 1H), 7.11 (q, J=9.25 Hz, 2H), 6.99-7.05 (m, 2H), 6.71 (t, J=5.56 Hz, 1H), 5.83 (s, 1H), 5.76-5.80 (m, 1H), 5.47 (s, 1H), 4.48 (d, J=5.75 Hz, 2H), 3.04 (s, 2H), 2.82 (d, J=7.21 Hz, 2H), 2.45 (s, 3H), 2.37 (dt, J=13.51, 6.69 Hz, 1H), 1.67 (s, 6H), 0.99 (s, 3H), 0.97 (s, 3H). LC-MS: m/z 642.4 (M+H)⁺.

### 4-[5-[(3,4-difluorophenyl)methylcarbamoyl]-2-thienyl]-6-[2-(4-fluorophenyl)ethyl]-2-(isopropylamino)-5-(5-methyl-1,3,4-oxadiazol-2-yl)pyridine-3-carboxamide (Compound 182)

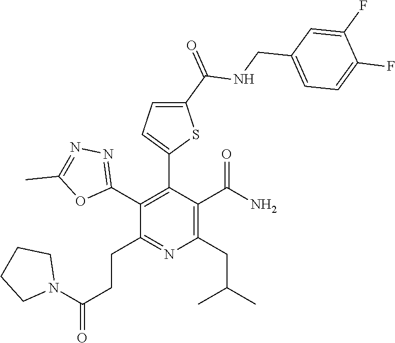 [SMILES: Cc1nnc(-c2c(CCC(=O)N3CCCC3)nc(CC(C)C)c(C(N)=O)c2-c2ccc(C(=O)NCc3ccc(F)c(F)c3)s2)o1]

Compound 182 was synthesized using a similar procedure described in the Example 34 above by using the appropriate materials.

¹H NMR (400 MHz, CDCl₃) δ ppm 7.37 (d, J=3.91 Hz, 1H), 7.03-7.21 (m, 3H), 6.94-7.01 (m, 1H), 6.70 (t, J=5.75 Hz, 1H), 6.22 (s, 1H), 5.79 (s, 1H), 4.51 (d, J=5.87 Hz, 2H), 3.52 (t, J=6.91 Hz, 2H), 3.40 (t, J=6.97 Hz, 2H), 3.05-3.13 (m, 2H), 2.84 (t, J=6.54 Hz, 2H), 2.79 (d, J=7.21 Hz, 2H), 2.46 (s, 3H), 2.30 (dt, J=13.63, 6.88 Hz, 1H), 2.00 (q, J=6.88 Hz, 2H), 1.85-1.90 (m, 2H), 0.95 (d, J=6.60 Hz, 6H). LC-MS: m/z 637.4 (M+H)⁺.

### Benzyl 4-[2-[5-carbamoyl-4-[5-[(3,4-difluorophenyl)methylcarbamoyl]-2-thienyl]-6-isobutyl-3-(5-methyl-1,3,4-oxadiazol-2-yl)-2-pyridyl]ethyl]piperidine-1-carboxylate (Compound 205)

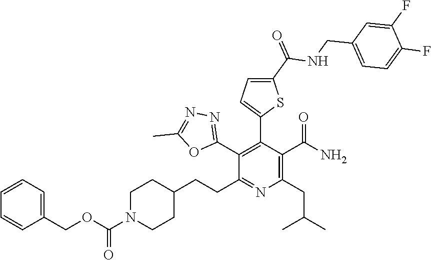 [SMILES: Cc1nnc(-c2c(CCC3CCN(C(=O)OCc4ccccc4)CC3)nc(CC(C)C)c(C(N)=O)c2-c2ccc(C(=O)NCc3ccc(F)c(F)c3)s2)o1]

Compound 205 was synthesized using a similar procedure described in the Example 34 above by using the appropriate materials.

¹H NMR (400 MHz, CDCl₃) δ ppm 7.33-7.36 (m, 6H), 6.99-7.19 (m, 5H), 6.50 (s, 1H), 5.74 (m, 1H), 5.58 (m, 1H), 5.11 (s, 2H), 4.51 (d, J=5.75 Hz, 2H), 4.14 (s, 2H), 2.82 (d, J=7.21 Hz, 2H), 2.69-2.79 (m, 4H), 2.46 (s, 3H), 2.35 (dt, J=13.51, 6.69 Hz, 1H), 1.18-1.46 (m, 4H), 1.08 (d, J=11.25 Hz, 3H), 0.98 (d, J=6.72 Hz, 6H). LC-MS: m/z 757.4 (M+H)⁺.

### Example 35

### (R)-4-(7-((6,7-dihydro-5H-cyclopenta[b]pyridin-5-yl)amino)thieno[2,3-c]pyridin-2-yl)-6-((4-fluorophenoxy)methyl)-2-isobutyl-5-(5-methyl-1,3,4-oxadiazol-2-yl)nicotinamide (Compound 173)

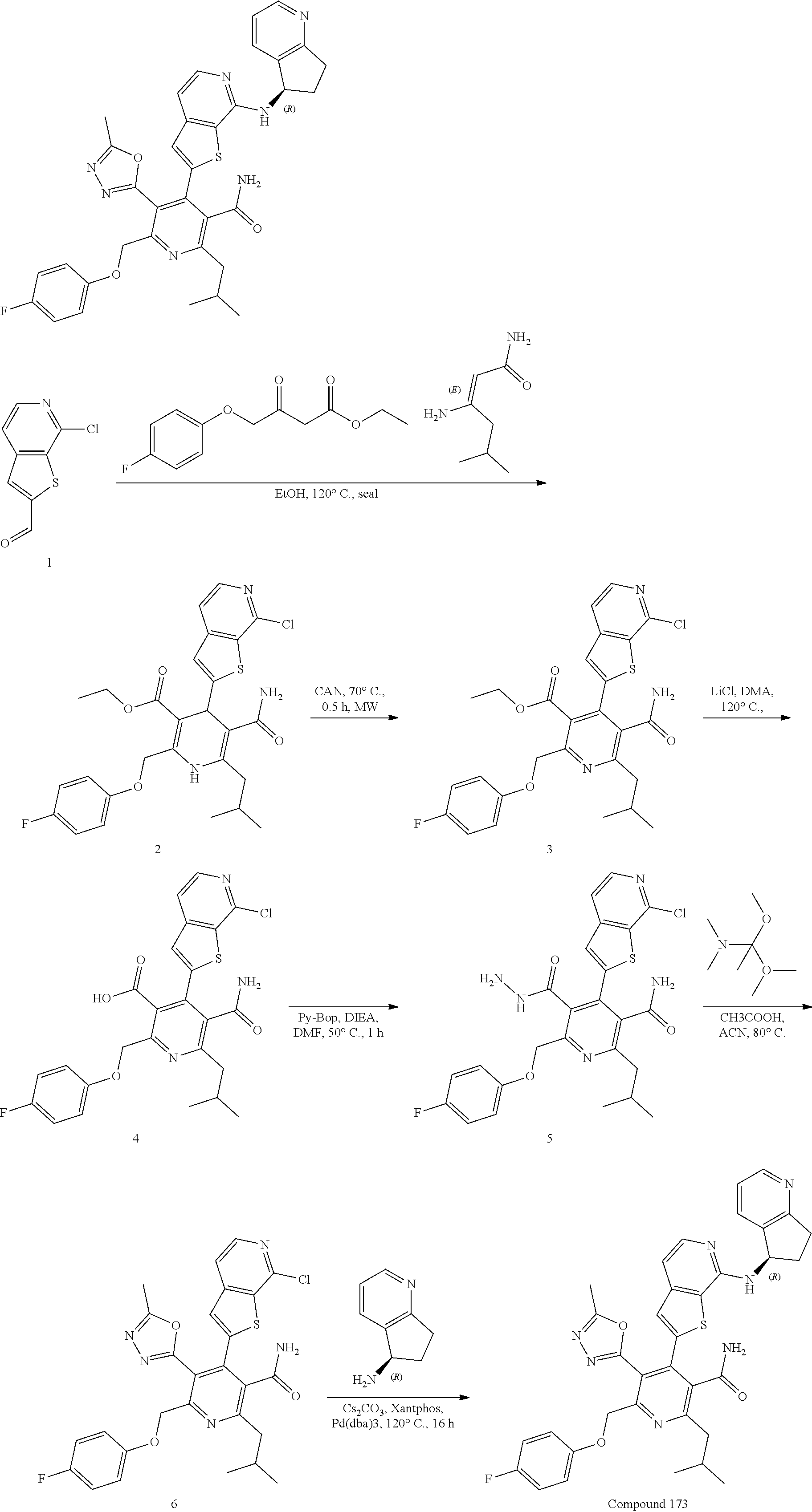

### Step A ethyl 5-carbamoyl-4-(7-chlorothieno [2,3-c]pyridin-2-yl)-2-((4-fluorophenoxy) methyl)-6-isobutyl-1,4-dihydropyridine-3-carboxylate

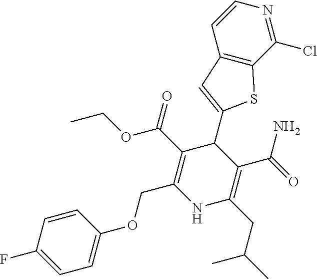 [SMILES: CCOC(=O)C1=C(COc2ccc(F)cc2)NC(CC(C)C)=C(C(N)=O)C1c1cc2ccnc(Cl)c2s1]

To a solution of 7-chlorothieno[2,3-c]pyridine-2-carbaldehyde (880 mg, 4.45 mmol) in EtOH (15 mL) were added ethyl 4-[(4-fluorophenyl)oxy]-3-oxobutanoate (1070 mg, 4.45 mmol), (2E)-3-amino-5-methylhex-2-enamide (633 mg, 4.45 mmol) and the reaction was stirred at 120° C. for overnight with N2 protection. The reaction was concentrated in vacuum to afford the compound ethyl 5-carbamoyl-4-(7-chlorothieno [2,3-c]pyridin-2-yl)-2-{[(4-fluorophenyl)oxy]methyl}-6-(2-methylpropyl)-1,4-dihydropyridine-3-carboxylate (1.5 g crude). LC-MS: m/z 544.1 (M+H)⁺.

### Step B ethyl 5-carbamoyl-4-(7-chlorothieno [2,3-c]pyridin-2-yl)-2-((4-fluorophenoxy)methyl)-6-isobutylnicotinate

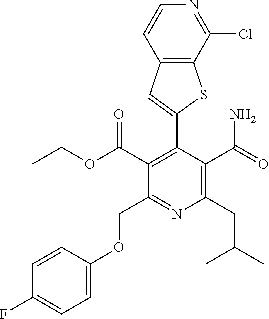 [SMILES: CCOC(=O)c1c(COc2ccc(F)cc2)nc(CC(C)C)c(C(N)=O)c1-c1cc2ccnc(Cl)c2s1]

To a solution of ethyl 5-carbamoyl-4-(7-chlorothieno[2,3-c]pyridin-2-yl)-2-{[(4-fluorophenyl)oxy]methyl}-6-(2-methylpropyl)-1,4-dihydropyridine-3-carboxylate (1.5 g, 2.76 mmol) in DCM (15 mL) were added ceric ammonium nitrate (4.53 g, 8.27 mmol) and the reaction was stirred at 70° C. for 3 hours. The reaction was filtered and concentrated in vacuum. The residue was purified using silica gel column chromatography eluting with EtOAc:PE=1:1 and concentrated in vacuum to afford the compound ethyl 5-carbamoyl-4-(7-chlorothieno[2,3-c]pyridin-2-yl)-2-{[(4-fluorophenyl)oxy]methyl}-6-(2-methylpropyl)pyridine-3-carboxylate (1.1 g, yield: 73.61%). LC-MS: m/z 542.22 (M+H)⁺.

### Step C 5-carbamoyl-4-(7-chlorothieno[2,3-c]pyridin-2-yl)-2-((4-fluorophenoxy)methyl)-6-isobutylnicotinic acid

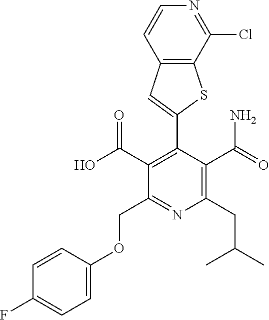 [SMILES: CC(C)Cc1nc(COc2ccc(F)cc2)c(C(=O)O)c(-c2cc3ccnc(Cl)c3s2)c1C(N)=O]

To a solution of ethyl 5-carbamoyl-4-(7-chlorothieno[2,3-c]pyridin-2-yl)-2-{[(4-fluorophenyl)oxy]methyl}-6-(2-methylpropyl)pyridine-3-carboxylate (1.06 g, 1.96 mmol) in DMA (15 mL) were added LiCl (1.24 g, 29.34 mmol) and the reaction was stirred at 150° C. for 6 hours. The reaction was diluted with EtOAc and brine. The organic layer was separated, washed with further brine, and concentrated in vacuum. The residue was purified using reversed phase column with 0.1% FA to afford the compound 5-carbamoyl-4-(7-chlorothieno[2,3-c]pyridin-2-yl)-2-{[(4-fluorophenyl)oxy]methyl}-6-(2-methylpropyl)pyridine-3-carboxylic acid (570 mg, yield: 56.71%). LC-MS: m/z 514.1 (M+H)⁺.

### Step D 4-(7-chlorothieno[2,3-c]pyridin-2-yl)-6-((4-fluorophenoxy)methyl)-5-(hydrazinecarbonyl)-2-isobutylnicotinamide

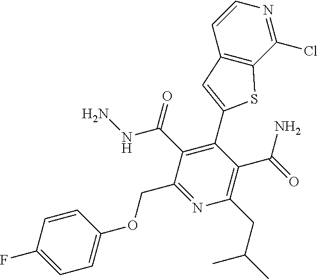 [SMILES: CC(C)Cc1nc(COc2ccc(F)cc2)c(C(=O)NN)c(-c2cc3ccnc(Cl)c3s2)c1C(N)=O]

To a solution of 5-carbamoyl-4-(7-chlorothieno[2,3-c]pyridin-2-yl)-2-{[(4-fluorophenyl)oxy]methyl}-6-(2-methylpropyl)pyridine-3-carboxylic acid (570 mg, 1.11 mmol) in DMF (5 mL) were added Hydrazine monohydrochloride (152 mg, 2.22 mmol), Benzotriazole-1-yl-oxytripyrrolidinophosphonium hexafluorophosphate (866 mg, 1.66 mmol), and DIEA (0.6 mL, 4.44 mmol), and the reaction was stirred at 50° C. for 2 hours. The reaction was diluted with EtOAc and brine. The organic layer was separated, washed with further brine, and concentrated in vacuum. The residue was filtered and concentrated in vacuum to afford the compound 4-(7-chlorothieno [2,3-c]pyridin-2-yl)-5-(diazanyl carbonyl)-6-{[(4-fluorophenyl) oxy] methyl}-2-(2-methylpropyl) pyridine-3-carboxamide (300 mg, yield: 51.23%). LC-MS: m/z 528.2 (M+H)⁺.

### Step E 4-(7-chlorothieno[2,3-c]pyridin-2-yl)-6-((4-fluorophenoxy)methyl)-2-isobutyl-5-(5-methyl-1,3,4-oxadiazol-2-yl)nicotinamide

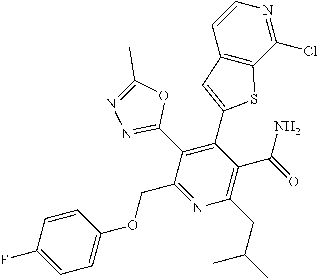 [SMILES: Cc1nnc(-c2c(COc3ccc(F)cc3)nc(CC(C)C)c(C(N)=O)c2-c2cc3ccnc(Cl)c3s2)o1]

To a solution of 4-(7-chlorothieno[2,3-c]pyridin-2-yl)-5-(diazanylcarbonyl)-6-{[(4-fluorophenyl)oxy]methyl}-2-(2-methylpropyl)pyridine-3-carboxamide (290 mg, 0.55 mmol) in ACN (2 mL) were added 1-(dimethylamino)-1,1-dimethoxyethane (81 mg, 0.60 mmol), after 1 hours, was added Acetic acid (2 mL) and the reaction was stirred at 80° C. for 3 hours. The reaction was diluted with EtOAc and brine. The organic layer was separated, washed with further brine, and concentrated in vacuum. The residue was purified using TLC with PE:EtOAc=1:2 and concentrated in vacuum to afford the compound 4-(7-chlorothieno[2,3-c]pyridin-2-yl)-6-{[(4-fluorophenyl)oxy]methyl}-5-(5-methyl-1,3,4-oxadiazol-2-yl)-2-(2-methylpropyl)pyridine-3-carboxamide (90 mg, yield: 29.68%). LC-MS: m/z 552.1 (M+H)⁺.

### Step F (R)-4-(7-((6,7-dihydro-5H-cyclopenta[b]pyridin-5-yl)amino)thieno[2,3-c]pyridin-2-yl)-6-((4-fluorophenoxy)methyl)-2-isobutyl-5-(5-methyl-1,3,4-oxadiazol-2-yl)nicotinamide

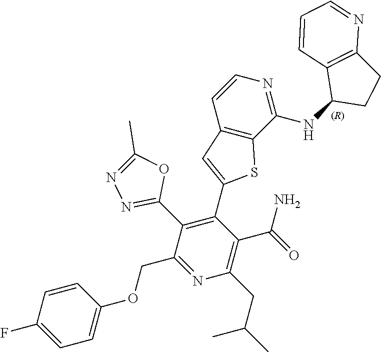

To a solution of 4-(7-chlorothieno[2,3-c]pyridin-2-yl)-6-{[(4-fluorophenyl)oxy]methyl}-5-(5-methyl-1,3,4-oxadiazol-2-yl)-2-(2-methylpropyl)pyridine-3-carboxamide (90 mg, 0.16 mmol) in Toluene (4 mL) were added (5R)-6,7-dihydro-5H-cyclopenta[1,2-b]pyridin-5-amine (66 mg, 0.49 mmol), Cs₂CO₃ (159 mg, 0.49 mmol), Xantphos (19 mg, 0.033 mmol), and Pd(dba)₃ (13 mg, 0.02 mmol), and the reaction was stirred at 120° C. for overnight in a seal tube with N₂ protection. The residue was purified by Pre-HPLC with 0.1% FA to afford the compound 6-{[(4-fluorophenyl)oxy]methyl}-4-(7-{[(5R)-6,7-dihydro-5H-cyclopenta[1,2-b]pyridin-5-yl]amino}thieno[2,3-c]pyridin-2-yl)-5-(5-methyl-1,3,4-oxadiazol-2-yl)-2-(2-methylpropyl)pyridine-3-carboxamide (1.07 mg, yield: 1.01%).

¹H NMR (400 MHz, Methanol-d₄): δ 8.33 (d, J=4.8 Hz, 1H), 7.94 (d, J=5.6 Hz, 1H), 7.69 (d, J=7.6 Hz, 1H), 7.32 (s, 1H), 7.18-7.21 (m, 1H), 7.06 (d, J=5.6 Hz, 1H), 6.93 (t, J=8.8 Hz, 2H), 6.74-6.77 (m, 2H), 5.87 (t, J=8.0 Hz, 1H), 5.32 (s, 2H), 3.10-3.13 (m, 1H), 2.99-3.05 (m, 1H), 2.88 (d, J=7.2 Hz, 2H), 2.68-2.73 (m, 1H), 2.37-2.42 (m, 1H), 2.31 (s, 3H), 2.06-2.11 (m, 1H), 1.00 (d, J=6.4 Hz, 6H). ¹⁹F NMR (Methanol-d₄): δ −125.21. LC-MS: m/z 650.2 (M+H)⁺.

### (R)-4-(7-((2,3-dihydro-1H-inden-1-yl)amino)thieno[2,3-c]pyridin-2-yl)-6-((4-fluorophenoxy)methyl)-2-isobutyl-5-(5-methyl-1,3,4-oxadiazol-2-yl)nicotinamide (Compound 175)

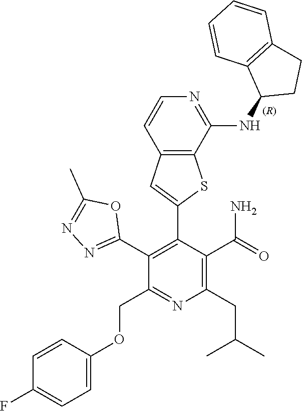

Compound 175 was synthesized using a similar procedure described in the Example 35 above by using the appropriate materials.

¹H NMR (Methanol-d4): δ 7.91 (d, J=5.6 Hz, 1H), 7.32 (s, 1H), 7.25-7.13 (m, 4H), 7.04 (d, J=5.6 Hz, 1H), 6.95-6.91 (m, 2H), 6.77-6.74 (m, 2H), 5.80 (t, J=7.6 Hz, 1H), 5.32 (s, 2H), 3.02-2.99 (m, 1H), 2.93-2.87 (m, 3H), 2.64-2.61 (m, 1H), 2.41-2.38 (m, 1H), 2.31 (s, 3H), 1.98-1.95 (m, 1H), 1.01 (d, J=6.8 Hz, 6H). ¹⁹F NMR (Methanol-d4): −125.21. LC-MS: m/z 649.4 (M+H)⁺.

### Example 36

### 4-(5-((3,4-difluorobenzyl)carbamoyl)thiophen-2-yl)-2-isobutyl-5-(5-methyl-1,3,4-oxadiazol-2-yl)-6-(2-(piperidin-1-yl)ethyl)nicotinamide (Compound 179)

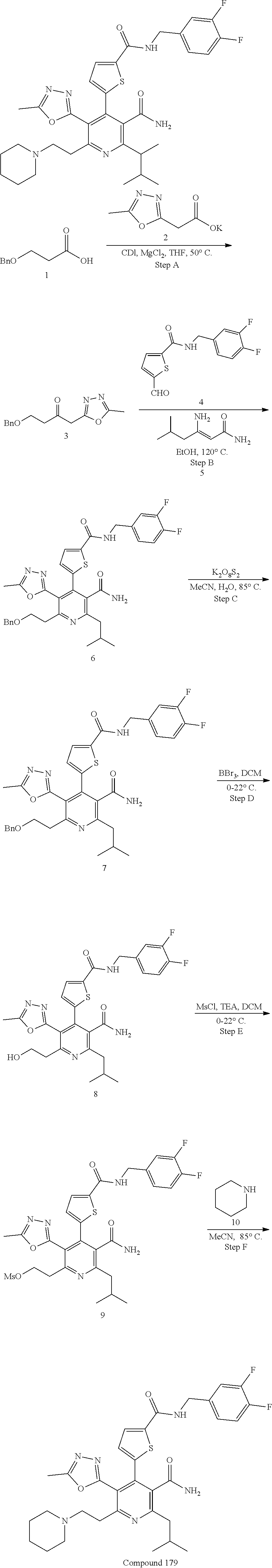

### Step A 4-benzyloxy-1-(5-methyl-1,3,4-oxadiazol-2-yl)butan-2-one

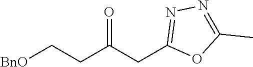 [SMILES: Cc1nnc(CC(=O)CCOCc2ccccc2)o1]

To a solution of 3-(benzyloxy)propanoic acid (899.99 mg, 4.99 mmol) in THF (10 mL) was added CDI (971.80 mg, 5.99 mmol). The mixture was stirred at 25° C. for 30 min and marked as Part A. To a mixture of potassium 2-(5-methyl-1,3,4-oxadiazol-2-yl)acetate (1.8 g, 9.99 mmol) in THF (10 mL) was added MgCl₂ (475.51 mg, 4.99 mmol) at 25° C. The mixture was stirred at 25° C. for 30 min and marked as Part B. Part A solution was added drop-wise to Part B over a period of 2 min at 25° C. and the mixture was stirred at 50° C. for 16 hrs. LC-MS showed Reactant 1 was consumed completely and one main peak with desired m/z or desired mass was detected. The reaction mixture was concentrated under reduced pressure to remove THF. The residue was diluted with water (20 mL) and extracted with EA (15 mL×2). The combined organic layer was washed with brine (50 mL×2), dried over Na₂SO₄, filtered and concentrated under reduced pressure to give a residue. The residue was purified by column chromatography (SiO₂, Petroleum ether/Ethyl acetate=0/1 to 1/1). 4-benzyloxy-1-(5-methyl-1,3,4-oxadiazol-2-yl)butan-2-one (870 mg, 3.34 mmol, 66.92% yield) was obtained. LC-MS: m/z 261.0 (M+H)⁺

### Step B 6-(2-(benzyloxy)ethyl)-4-(5-((3,4-difluorobenzyl)carbamoyl)thiophen-2-yl)-2-isobutyl-5-(5-methyl-1,3,4-oxadiazol-2-yl)-1,4-dihydropyridine-3-carboxamide

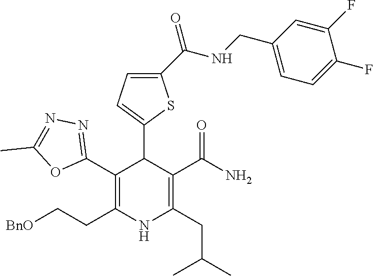 [SMILES: Cc1nnc(C2=C(CCOCc3ccccc3)NC(CC(C)C)=C(C(N)=O)C2c2ccc(C(=O)NCc3ccc(F)c(F)c3)s2)o1]

A mixture of 4-benzyloxy-1-(5-methyl-1,3,4-oxadiazol-2-yl)butan-2-one (600 mg, 2.31 mmol), (Z)-3-amino-5-methyl-hex-2-enamide (340 mg, 2.39 mmol), NH₄OAc (360 mg, 4.67 mmol), N-[(3,4-difluorophenyl)methyl]-5-formyl-thiophene-2-carboxamide (660 mg, 2.35 mmol) in EtOH (3 mL) was degassed and purged with N₂ for 3 times, and then the mixture was stirred at 120° C. for 16 hrs under N₂ atmosphere. The reaction mixture was concentrated under reduced pressure to remove solvent. The crude product was purified by column chromatography on silica gel (eluted with 0˜80% EA in PE). 6-(2-benzyloxyethyl)-4-[5-[(3,4-difluorophenyl)methylcarbamoyl]-2-thienyl]-2-isobutyl-5-(5-methyl-1,3,4-oxadiazol-2-yl)-1,4-dihydropyridine-3-carboxamide (550 mg, 679.29 μmol) was obtained. LC-MS: m/z 649.6 (M+H)⁺

### Step C 6-(2-(benzyloxy)ethyl)-4-(5-((3,4-difluorobenzyl)carbamoyl)thiophen-2-yl)-2-isobutyl-5-(5-methyl-1,3,4-oxadiazol-2-yl)nicotinamide

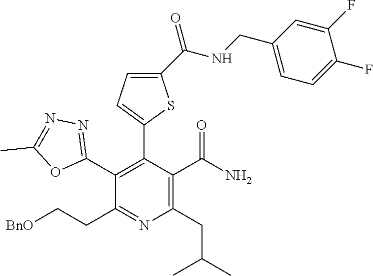 [SMILES: Cc1nnc(-c2c(CCOCc3ccccc3)nc(CC(C)C)c(C(N)=O)c2-c2ccc(C(=O)NCc3ccc(F)c(F)c3)s2)o1]

To a solution of 6-(2-benzyloxyethyl)-4-[5-[(3,4-difluorophenyl)methylcarbamoyl]-2-thienyl]-2-isobutyl-5-(5-methyl-1,3,4-oxadiazol-2-yl)-1,4-dihydropyridine-3-carboxamide (500 mg, 771.92 μmol) in MeCN (9 mL) and H₂O (3 mL) was added dipotassium sulfonatooxy sulfate (210.00 mg, 776.85 μmol). The mixture was stirred at 85° C. for 2 hrs. The reaction mixture was concentrated under reduced pressure to remove solvent. The crude product was purified by column chromatography on silica gel (eluted with 0˜80% EA in PE). 6-(2-benzyloxyethyl)-4-[5-[(3,4-difluorophenyl)methylcarbamoyl]-2-thienyl]-2-isobutyl-5-(5-methyl-1,3,4-oxadiazol-2-yl)pyridine-3-carboxamide (257 mg, 378.11 μmol) was obtained. LC-MS: m/z 646.7 (M+H)⁺.

### Step D 4-(5-((3,4-difluorobenzyl)carbamoyl)thiophen-2-yl)-6-(2-hydroxyethyl)-2-isobutyl-5-(5-methyl-1,3,4-oxadiazol-2-yl)nicotinamide

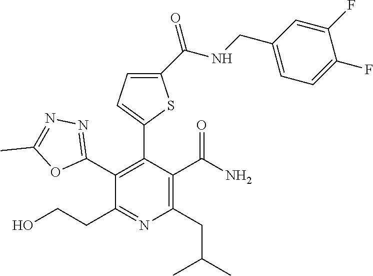 [SMILES: Cc1nnc(-c2c(CCO)nc(CC(C)C)c(C(N)=O)c2-c2ccc(C(=O)NCc3ccc(F)c(F)c3)s2)o1]

To a solution of 6-(2-benzyloxyethyl)-4-[5-[(3,4-difluorophenyl)methylcarbamoyl]-2-thienyl]-2-isobutyl-5-(5-methyl-1,3,4-oxadiazol-2-yl)pyridine-3-carboxamide (100 mg, 154.87 μmol) in DCM (5 mL) was added dropwise BBr₃ (2 M, 0.2 mL) at 0° C. After addition, the mixture was stirred at 22° C. for 16 hrs. The reaction mixture was quenched by aqueous NaHCO₃ (30 mL) at 0° C., and then diluted with H₂O (30 mL) and extracted with DCM (50 mL×2). The combined organic layer was washed with brine (50 mL), dried over Na₂SO₄, filtered and concentrated under reduced pressure to give a residue. The residue was purified by column chromatography on silica gel (eluted with 0˜80% EA in PE). Compound 4-[5-[(3,4-difluorophenyl)methylcarbamoyl]-2-thienyl]-6-(2-hydroxyethyl)-2-isobutyl-5-(5-methyl-1,3,4-oxadiazol-2-yl)pyridine-3-carboxamide (56 mg, 95.75 μmol, 61.83% yield, 95% purity) was obtained. LC-MS: m/z 556.5 (M+H)⁺

### Step E 2-(5-carbamoyl-4-(5-((3,4-difluorobenzyl)carbamoyl)thiophen-2-yl)-6-isobutyl-3-(5-methyl-1,3,4-oxadiazol-2-yl)pyridin-2-yl)ethyl methanesulfonate

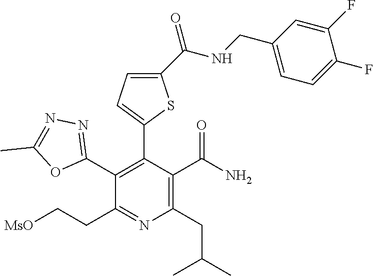 [SMILES: Cc1nnc(-c2c(CCOS(C)(=O)=O)nc(CC(C)C)c(C(N)=O)c2-c2ccc(C(=O)NCc3ccc(F)c(F)c3)s2)o1]

To a solution of 4-[5-[(3,4-difluorophenyl)methylcarbamoyl]-2-thienyl]-6-(2-hydroxyethyl)-2-isobutyl-5-(5-methyl-1,3,4-oxadiazol-2-yl)pyridine-3-carboxamide (56 mg, 100.79 μmol) and TEA (36.35 mg, 359.23 μmol) in DCM (4 mL) was added MsCl (0.14 g, 1.22 mmol) at 0° C. The mixture was stirred at 22° C. for 2 hrs. The reaction mixture was quenched by H₂O (50 mL) at 0° C., and then extracted with EA (50 mL). The combined organic layer was washed with brine (30 mL), dried over Na₂SO₄, filtered and concentrated under reduced pressure to give a residue. Compound 2-[5-carbamoyl-4-[5-[(3,4-difluorophenyl)methylcarbamoyl]-2-thienyl]-6-isobutyl-3-(5-methyl-1,3,4-oxadiazol-2-yl)-2-pyridyl]ethyl methanesulfonate (80 mg, crude) was obtained, which was used directly in the next step.

### Step F 4-(5-((3,4-difluorobenzyl)carbamoyl)thiophen-2-yl)-2-isobutyl-5-(5-methyl-1,3,4-oxadiazol-2-yl)-6-(2-(piperidin-1-yl)ethyl)nicotinamide

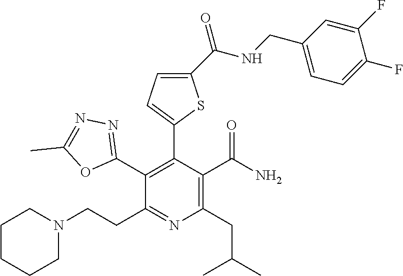 [SMILES: Cc1nnc(-c2c(CCN3CCCCC3)nc(CC(C)C)c(C(N)=O)c2-c2ccc(C(=O)NCc3ccc(F)c(F)c3)s2)o1]

A mixture of 2-[5-carbamoyl-4-[5-[(3,4-difluorophenyl)methylcarbamoyl]-2-thienyl]-6-isobutyl-3-(5-methyl-1,3,4-oxadiazol-2-yl)-2-pyridyl]ethyl methanesulfonate (64 mg, 101.00 μmol), piperidine (25.80 mg, 302.99 μmol) in MeCN (5 mL) was degassed and purged with N₂ for 3 times, and then the mixture was stirred at 85° C. for 16 hrs under N₂ atmosphere. The reaction mixture was concentrated under reduced pressure to remove solvent. The residue was purified by prep-TLC (SiO₂, DCM:MeOH=10:1). Compound 4-[5-[(3,4-difluorophenyl)methylcarbamoyl]-2-thienyl]-2-isobutyl-5-(5-methyl-1,3,4-oxadiazol-2-yl)-6-[2-(1-piperidyl)ethyl]pyridine-3-carboxamide (3.28 mg, 5.21 μmol, 5.15% yield, 98.82% purity) was obtained.

¹H NMR (400 MHz, CDCl₃) δ ppm 7.36 (d, J=3.67 Hz, 1H), 6.92-7.19 (m, 5H), 6.06-6.24 (m, 1H), 5.94 (s, 1H), 4.47 (d, J=5.87 Hz, 2H), 2.94-3.03 (m, 2H), 2.74-2.93 (m, 4H), 2.46-2.61 (m, 3H), 2.43 (s, 3H), 2.32-2.38 (m, 1H), 1.60 (m, 5H), 1.41-1.47 (m, 2H), 0.98 (d, J=6.60 Hz, 6H). LC-MS: m/z 623.2 (M+H)⁺.

### Example 37

### 4-(5-(((R)-2,3-dihydro-1H-inden-1-yl)carbamoyl)thiophen-2-yl)-6-(2-(4-fluorophenyl)-2-hydroxyethyl)-2-isobutyl-5-(5-methyl-1,3,4-oxadiazol-2-yl)nicotinamide (Compound 185) and 4-(5-(((R)-2,3-dihydro-1H-inden-1-yl)carbamoyl)thiophen-2-yl)-6-(2-fluoro-2-(4-fluorophenyl)ethyl)-2-isobutyl-5-(5-methyl-1,3,4-oxadiazol-2-yl)nicotinamide (Compound 187)

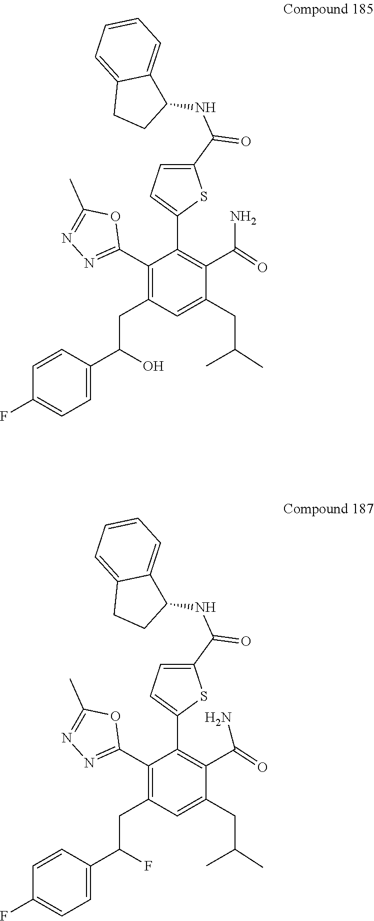 [SMILES: Cc1nnc(-c2c(CC(O)c3ccc(F)cc3)cc(CC(C)C)c(C(N)=O)c2-c2ccc(C(=O)N[C@@H]3CCc4ccccc43)s2)o1 ; Cc1nnc(-c2c(CC(F)c3ccc(F)cc3)cc(CC(C)C)c(C(N)=O)c2-c2ccc(C(=O)N[C@@H]3CCc4ccccc43)s2)o1]

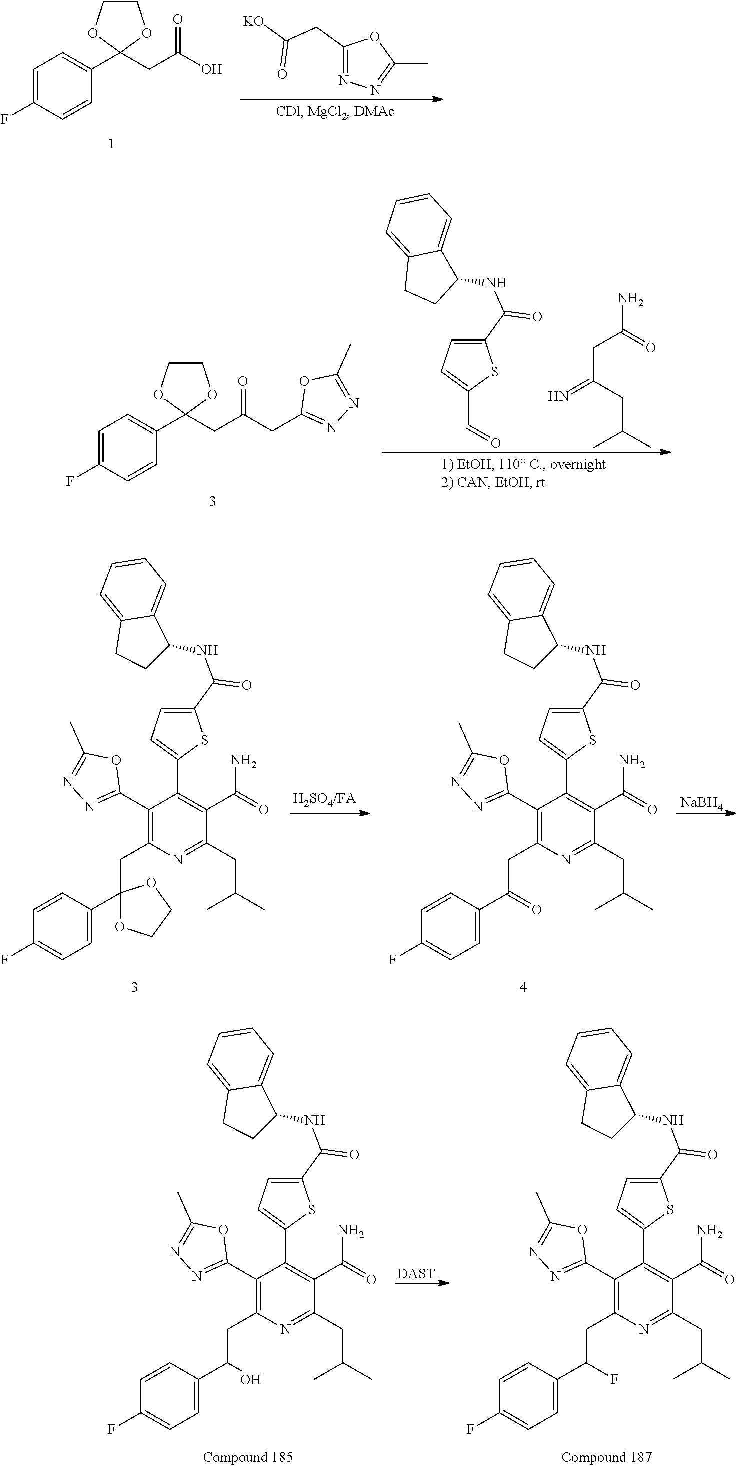

### Step A 1-[2-(4-fluorophenyl)-1,3-dioxolan-2-yl]-3-(5-methyl-1,3,4-oxadiazol-2-yl)propan-2-one

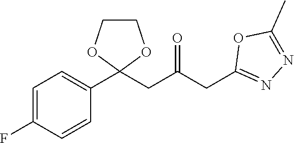 [SMILES: Cc1nnc(CC(=O)CC2(c3ccc(F)cc3)OCCO2)o1]

To a solution of [2-(4-fluorophenyl)-1,3-dioxolan-2-yl]acetic acid (1.6 g, 7.073 mmol) in DMA (25 mL) were added CDI (1.72 g, 10.610 mmol) and the reaction was stirred at room temperature for 1 hr. Then {[2-(5-methyl-1,3,4-oxadiazol-2-yl)acetyl]oxy}potassium (1.27 g, 7.073 mmol) and MgCl₂ (0.78 g, 8.488 mmol) was added and the reaction was stirred at 50° C. for 16 h. The reaction was diluted with EA (100 mL) and water (80 mL). The organic layer was separated, washed with further saturated NaCl solution, and concentrated in vacuo. The residue was purified using silica gel column chromatography eluting with ethyl acetate in petroleum ether (PE:EA=3:1). The organic layer was concentrated in vacuo to afford the title compound 1-[2-(4-fluorophenyl)-1,3-dioxolan-2-yl]-3-(5-methyl-1,3,4-oxadiazol-2-yl)propan-2-one (1.8 g, 5.877 mmol, 83.08%). LC-MS: m/z 307.1 (M+H)⁺.

### Step B (R)-4-(5-((2,3-dihydro-1H-inden-1-yl)carbamoyl)thiophen-2-yl)-6-((2-(4-fluorophenyl)-1,3-dioxolan-2-yl)methyl)-2-isobutyl-5-(5-methyl-1,3,4-oxadiazol-2-yl)nicotinamide

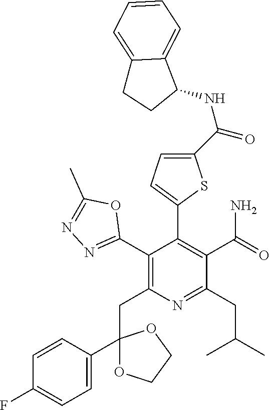 [SMILES: Cc1nnc(-c2c(CC3(c4ccc(F)cc4)OCCO3)nc(CC(C)C)c(C(N)=O)c2-c2ccc(C(=O)N[C@@H]3CCc4ccccc43)s2)o1]

To a solution of 1-[2-(4-fluorophenyl)-1,3-dioxolan-2-yl]-3-(5-methyl-1,3,4-oxadiazol-2-yl)propan-2-one (600 mg, 1.959 mmol) in EtOH (10 mL) were added N-(2,3-dihydro-1H-indenyl)-5-formylthiophene-2-carboxamide (531.52 mg, 1.959 mmol) and 3-azanylidene-5-methylhexanamide (306.42 mg, 2.155 mmol). The reaction was stirred at 110° C. for 18 hr. Then cerium(IV) ammonium nitrate (2.24 g, 4.095 mmol) was added at room temperature and the reaction was stirred at room temperature for 1 hr. The reaction was diluted with EA (100 mL) and water (80 mL). The organic layer was separated, washed with further saturated NaCl solution, and concentrated in vacuo. The residue was purified using silica gel column chromatography eluting with ethyl acetate in petroleum ether (PE:EA=1:1). The organic layer was concentrated in vacuo to afford the title compound (R)-4-(5-((2,3-dihydro-1H-inden-1-yl)carbamoyl)thiophen-2-yl)-6-((2-(4-fluorophenyl)-1,3-dioxolan-2-yl)methyl)-2-isobutyl-5-(5-methyl-1,3,4-oxadiazol-2-yl)nicotinamide (0.9 g, 1.320 mmol, 64.48%). LC-MS: m/z 682.2 (M+H)⁺.

### Step C (R)-4-(5-((2,3-dihydro-1H-inden-1-yl)carbamoyl)thiophen-2-yl)-6-(2-(4-fluorophenyl)-2-oxoethyl)-2-isobutyl-5-(5-methyl-1,3,4-oxadiazol-2-yl)nicotinamide

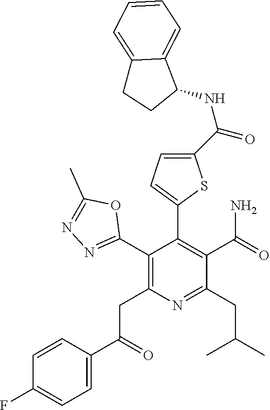 [SMILES: Cc1nnc(-c2c(CC(=O)c3ccc(F)cc3)nc(CC(C)C)c(C(N)=O)c2-c2ccc(C(=O)N[C@@H]3CCc4ccccc43)s2)o1]

To a solution of (R)-4-(5-((2,3-dihydro-1H-inden-1-yl)carbamoyl)thiophen-2-yl)-6-((2-(4-fluorophenyl)-1,3-dioxolan-2-yl)methyl)-2-isobutyl-5-(5-methyl-1,3,4-oxadiazol-2-yl)nicotinamide (450 mg, 0.660 mmol) in FA (5 mL) were added conc. H₂SO₄ (0.177 mL, 3.300 mmol), and the reaction was stirred at 45° C. for 4 hr. The reaction was diluted with EA (100 mL) and water (80 mL). The organic layer was separated, washed with further saturated NaCl solution, and concentrated in vacuo. The residue was purified using silica gel column chromatography eluting with ethyl acetate in petroleum ether. The organic layer was concentrated in vacuo to afford the title compound (R)-4-(5-((2,3-dihydro-1H-inden-1-yl)carbamoyl)thiophen-2-yl)-6-(2-(4-fluorophenyl)-2-oxoethyl)-2-isobutyl-5-(5-methyl-1,3,4-oxadiazol-2-yl)nicotinamide (100 mg, 0.157 mmol, 23.76%). LC-MS: m/z 638.0 (M+H)⁺.

### Step D 4-(5-(((R)-2,3-dihydro-1H-inden-1-yl)carbamoyl)thiophen-2-yl)-6-(2-(4-fluorophenyl)-2-hydroxyethyl)-2-isobutyl-5-(5-methyl-1,3,4-oxadiazol-2-yl)nicotinamide

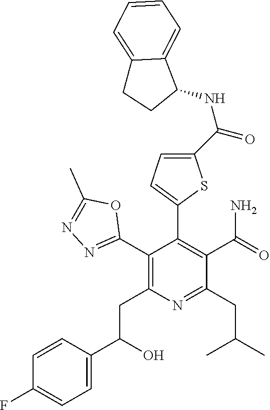 [SMILES: Cc1nnc(-c2c(CC(O)c3ccc(F)cc3)nc(CC(C)C)c(C(N)=O)c2-c2ccc(C(=O)N[C@@H]3CCc4ccccc43)s2)o1]

To a solution of (R)-4-(5-((2,3-dihydro-1H-inden-1-yl)carbamoyl)thiophen-2-yl)-6-(2-(4-fluorophenyl)-2-oxoethyl)-2-isobutyl-5-(5-methyl-1,3,4-oxadiazol-2-yl)nicotinamide (90 mg, 0.141 mmol) in MeOH (1 mL) and THF (1 mL) were added NaBH₄ (5.34 mg, 0.141 mmol) at 0° C., and the reaction was stirred at 0° C. for 1 hr. The reaction was diluted with EA (50 mL) and water (50 mL). The organic layer was separated, washed with further saturated NaCl solution, and concentrated in vacuo. The residue was purified Pre-HPLC to afford (60 mg, 0.094 mmol, 66.45%).

¹H NMR (400 MHz, DMSO-d₆): δ 8.85 (d, J=8.4 Hz, 1H), 7.87 (br s, 1H), 7.66 (d, J=3.6 Hz, 1H), 7.58 (br s, 1H), 7.12-7.28 (m, 6H), 7.05-7.09 (m, 2H), 6.92 (d, J=4.0 Hz, 1H), 5.47 (q, J=8.0 Hz, 1H), 5.39 (d, J=4.4 Hz, 1H), 4.93-4.97 (m, 1H), 3.12-3.16 (m, 1H), 3.03-3.09 (m, 1H), 2.95-3.02 (m, 1H), 2.80-2.88 (m, 1H), 2.71 (d, J=6.8 Hz, 2H), 2.40-2.46 (m, 1H), 2.37 (s, 3H), 2.22-2.33 (m, 1H), 1.88-1.98 (m, 1H), 0.93 (d, J=6.8 Hz, 6H). ¹⁹F NMR (377 MHz, DMSO-d6): −115.96. LC-MS: m/z 640.3 (M+H)⁺.

### Step E 4-(5-(((R)-2,3-dihydro-1H-inden-1-yl)carbamoyl)thiophen-2-yl)-6-(2-fluoro-2-(4-fluorophenyl)ethyl)-2-isobutyl-5-(5-methyl-1,3,4-oxadiazol-2-yl)nicotinamide

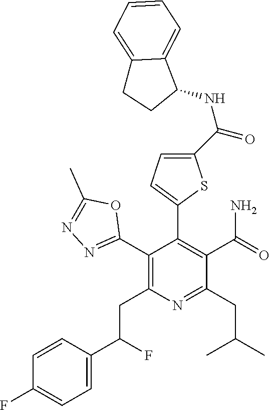 [SMILES: Cc1nnc(-c2c(CC(F)c3ccc(F)cc3)nc(CC(C)C)c(C(N)=O)c2-c2ccc(C(=O)N[C@@H]3CCc4ccccc43)s2)o1]

To a solution of 4-(5-(((R)-2,3-dihydro-1H-inden-1-yl)carbamoyl)thiophen-2-yl)-6-(2-(4-fluorophenyl)-2-hydroxyethyl)-2-isobutyl-5-(5-methyl-1,3,4-oxadiazol-2-yl)nicotinamide (27 mg, 0.042 mmol) in DCM (1 mL) were added DAST (6.80 mg, 0.042 mmol) at 0° C., and the reaction was stirred at room temperature for 1 hr. The reaction was diluted with EA (50 mL), washed with further water, and concentrated in vacuo. The residue was purified Pre-HPLC to afford the title compound (2.1 mg, 0.003 mmol, 7.75%).

¹H NMR (400 MHz, DMSO-d₆): δ 8.87 (d, J=8.4 Hz, 1H), 7.93 (br s, 1H), 7.68 (d, J=2.8 Hz, 1H), 7.63 (br s, 1H), 7.35-7.43 (m, 2H), 7.14-7.31 (m, 6H), 6.96 (d, J=3.2 Hz, 1H), 5.94-6.14 (m, 1H), 5.44-5.50 (m, 1H), 3.38-3.48 (m, 2H), 2.93-3.04 (m, 1H), 2.74-2.90 (m, 1H), 2.71 (d, J=15.6 Hz, 2H), 2.34-2.45 (m, 4H), 2.21-2.27 (m, 1H), 1.86-2.02 (m, 1H), 0.92 (d, J=6.8 Hz, 6H). ¹⁹F NMR (377 MHz, DMSO-d₆): −113.26, −168.94. LC-MS: m/z 642.0 (M+H)⁺.

### Example 38

### 4-(4-((3,4-difluorobenzyl)carbamoyl)phenyl)-6-ethyl-2-isobutyl-5-(5-methyl-1,3,4-oxadiazol-2-yl)nicotinamide (Compound 199)

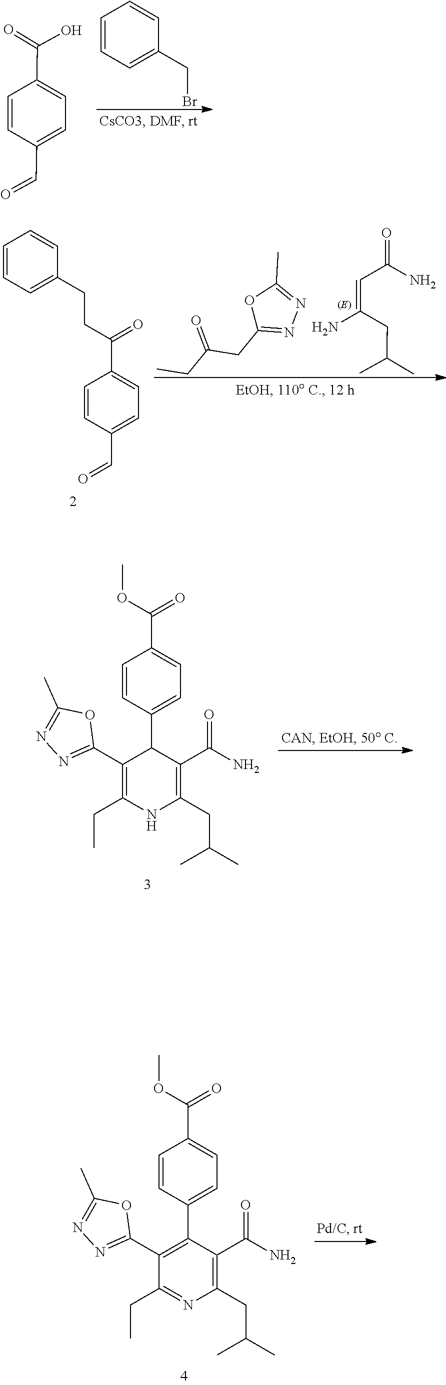

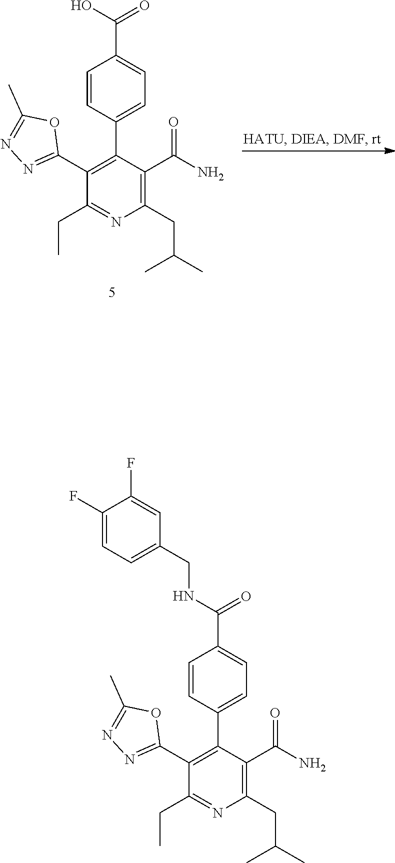

### Step A benzyl 4-formylbenzoate

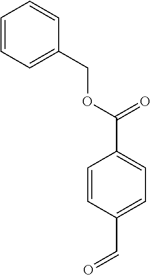 [SMILES: O=Cc1ccc(C(=O)OCc2ccccc2)cc1]

To a mixture of 4-formylbenzoic acid (2 g, 13.322 mmol), (bromomethyl)benzene (3.169 mL, 26.644 mmol) and dicaesium carbonate (13.02 g, 39.965 mmol) in DMF (15 mL) was stirred at room temperature for 3 hours. The reaction was filtered through a Celite pad, and the filtrate was diluted with H₂O (70 mL) and extracted with EA (50 mL×3), the organic layer dried over sodium sulfate, filtered and concentrated in vacuum. The reaction was purified by column chromatography (PE/EA=3/1) to afford benzyl 4-formylbenzoate (2.8 g, 11.654 mmol, 87.50%).

¹H NMR (400 MHz, DMSO-d6): δ 10.12 (s, 1H), 8.19 (d, J=7.2 Hz, 2H), 8.06 (d, J=8.0 Hz, 2H), 7.51 (d, J=7.2 Hz, 2H), 7.37-7.45 (m, 3H), 5.40 (s, 2H)

### Step B benzyl 4-(3-carbamoyl-6-ethyl-2-isobutyl-5-(5-methyl-1,3,4-oxadiazol-2-yl)-1,4-dihydropyridin-4-yl)benzoate

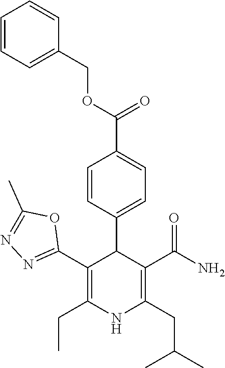 [SMILES: CCC1=C(c2nnc(C)o2)C(c2ccc(C(=O)OCc3ccccc3)cc2)C(C(N)=O)=C(CC(C)C)N1]

To a mixture of benzyl 4-formylbenzoate (467.52 mg, 1.946 mmol), (E)-3-amino-5-methylhex-2-enamide (276.71 mg, 1.946 mmol) and 1-(5-methyl-1,3,4-oxadiazol-2-yl)butan-2-one (300 mg, 1.946 mmol) in EtOH (4 mL) of sealed tube was stirred at 110° C. overnight. The reaction was concentrated to give the crude product and purified by column chromatography (PE/EA=1/3) to afford benzyl 4-(3-carbamoyl-6-ethyl-2-isobutyl-5-(5-methyl-1,3,4-oxadiazol-2-yl)-1,4-dihydropyridin-4-yl)benzoate (850 mg, 1.698 mmol, 87.26%). LC-MS: m/z 501.3 (M+H)⁺.

### Step C benzyl 4-(3-carbamoyl-6-ethyl-2-isobutyl-5-(5-methyl-1,3,4-oxadiazol-2-yl)pyridin-4-yl)benzoate

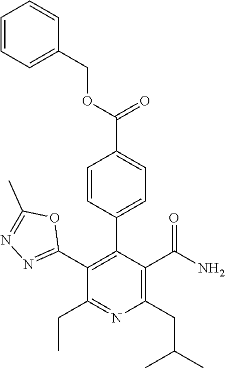 [SMILES: CCc1nc(CC(C)C)c(C(N)=O)c(-c2ccc(C(=O)OCc3ccccc3)cc2)c1-c1nnc(C)o1]

A mixture of benzyl 4-(3-carbamoyl-6-ethyl-2-isobutyl-5-(5-methyl-1,3,4-oxadiazol-2-yl)-1,4-dihydropyridin-4-yl)benzoate (700 mg, 1.398 mmol) and ceric ammonium nitrate (919.81 mg, 1.678 mmol) in EtOH (5 mL) was stirred at 50° C. for 2 hours. The reaction was concentrated to give the crude product and purified by reversed phase column chromatography (0.1% FA/H₂O/CH₃CN) to afford benzyl 4-(3-carbamoyl-6-ethyl-2-isobutyl-5-(5-methyl-1,3,4-oxadiazol-2-yl)pyridin-4-yl)benzoate (400 mg, 0.802 mmol, 57.37%). LC-MS: m/z 499.2 (M+H)⁺.

### Step D 4-(3-carbamoyl-6-ethyl-2-isobutyl-5-(5-methyl-1,3,4-oxadiazol-2-yl)pyridin-4-yl)benzoic acid

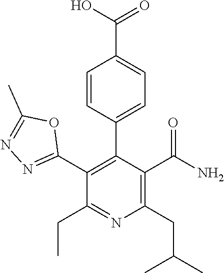 [SMILES: CCc1nc(CC(C)C)c(C(N)=O)c(-c2ccc(C(=O)O)cc2)c1-c1nnc(C)o1]

To a mixture of benzyl 4-(3-carbamoyl-6-ethyl-2-isobutyl-5-(5-methyl-1,3,4-oxadiazol-2-yl)pyridin-4-yl)benzoate (300 mg, 0.602 mmol) and Pd/C (128.07 mg, 10% Pd, wetted with ca. 55% water) in THF (3 mL) was stirred at room temperature for 1 hour. The reaction was filtered through a Celite pad, and the filtrate was concentrated to give the crude product and purified by reversed phase column chromatography (0.1% FA/H₂O/CH₃CN) to afford 4-(3-carbamoyl-6-ethyl-2-isobutyl-5-(5-methyl-1,3,4-oxadiazol-2-yl)pyridin-4-yl)benzoic acid (240 mg, 0.588 mmol, 97.65%). LC-MS: m/z 409.2 (M+H)⁺.

### 4-(4-((3,4-difluorobenzyl)carbamoyl)phenyl)-6-ethyl-2-isobutyl-5-(5-methyl-1,3,4-oxadiazol-2-yl)nicotinamide

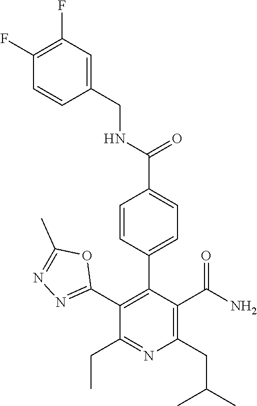 [SMILES: CCc1nc(CC(C)C)c(C(N)=O)c(-c2ccc(C(=O)NCc3ccc(F)c(F)c3)cc2)c1-c1nnc(C)o1]

To a solution of 4-(3-carbamoyl-6-ethyl-2-isobutyl-5-(5-methyl-1,3,4-oxadiazol-2-yl)pyridin-4-yl)benzoic acid (50 mg, 0.122 mmol) and (3,4-difluorophenyl)methanamine (26.28 mg, 0.184 mmol) in DMF (1 mL) were added DIEA (22.87 mg, 0.177 mmol) and HATU (69.82 mg, 0.184 mmol), and the reaction was stirred at room temperature for 1 hour. After the reaction was completed, the mixture was diluted with H₂O (30 mL), extracted with ethyl acetate (30 mL×3). The organic layers were combined, dried over Na₂SO₄, filtered and concentrated to crude product and purified by Prep-HPLC to give the title compound (32.95 mg, yield: 50.44%).

¹H NMR (400 MHz, DMSO-d₆): δ 9.11 (t, J=6.0 Hz, 1H), 7.75-7.82 (m, 3H), 7.46 (br s, 1H), 7.33-7.43 (m, 2H), 7.25 (d, J=8.4 Hz, 2H), 7.13-7.18 (m, 1H), 4.44 (d, J=6.0 Hz, 2H), 2.74 (d, J=7.6 Hz, 2H), 2.66 (q, J=7.6 Hz, 2H), 2.29-2.34 (m, 4H), 1.19 (t, J=7.6 Hz, 3H), 0.96 (d, J=6.4 Hz, 6H). ¹⁹F NMR (377 MHz, DMSO-d₆): δ −138.99, −141.59. LC-MS: m/z 534.2 (M+H)⁺.

The following compounds were synthesized using methods described herein.

### 4-(1-(3,4-difluorophenethyl)-1,2,3,4-tetrahydroquinolin-6-yl)-6-(4-fluorophenethyl)-2-isobutyl-5-(5-methyl-1,3,4-oxadiazol-2-yl)nicotinamide (Compound 174)

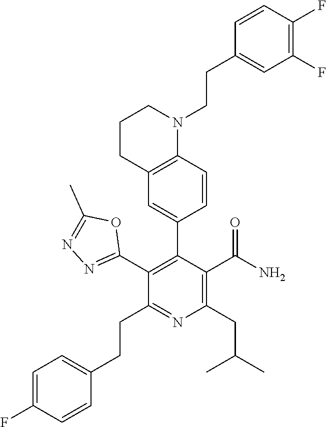 [SMILES: Cc1nnc(-c2c(CCc3ccc(F)cc3)nc(CC(C)C)c(C(N)=O)c2-c2ccc3c(c2)CCCN3CCc2ccc(F)c(F)c2)o1]

Compound 174 was synthesized using a similar procedure described in the Example 12 above by using 1-(3,4-difluorophenethyl)-1,2,3,4-tetrahydroquinoline-6-carbaldehyde and the appropriate materials.

¹H NMR (400 MHz, DMSO-d₆): δ 7.67 (br s, 1H), 7.30-7.41 (m, 3H), 7.02-7.12 (m, 5H), 6.81 (dd, J=8.4 Hz, J=2.4 Hz, 1H), 6.64 (d, J=2.4 Hz, 1H), 6.56 (d, J=8.8 Hz, 1H), 3.60 (t, J=7.2 Hz, 2H), 3.42 (t, J=7.2 Hz, 2H), 2.88-2.96 (m, 4H), 2.76 (t, J=7.2 Hz, 2H), 2.70 (d, J=7.2 Hz, 2H), 2.57-2.61 (m, 1H), 2.26-2.32 (m, 5H), 1.73-1.77 (m, 2H), 0.93 (d, J=6.4 Hz, 6H). ¹⁹F NMR (377 MHz, DMSO-d₆): −117.38, −139.29, 142.58. LC-MS: m/z 653.8 (M+H)⁺.

### 4-(1-((2,3-dihydro-1H-inden-1-yl)methyl)-1H-indol-5-yl)-6-(4-fluorophenethyl)-2-isobutyl-5-(5-methyl-1,3,4-oxadiazol-2-yl)nicotinamide (Compound 177)

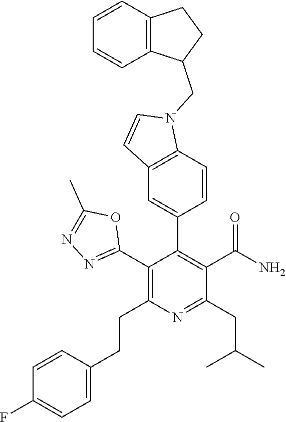 [SMILES: Cc1nnc(-c2c(CCc3ccc(F)cc3)nc(CC(C)C)c(C(N)=O)c2-c2ccc3c(ccn3CC3CCc4ccccc43)c2)o1]

Compound 177 was synthesized using a similar procedure described in the Example 20 above by using the appropriate materials.

¹H NMR (400 MHz, CDCl₃) δ ppm 7.46 (s, 1H), 7.17-7.26 (m, 3H), 7.10 (br s, 1H), 6.94-7.04 (m, 4H), 6.70-6.94 (m, 3H), 6.42 (d, J=2.93 Hz, 1H), 5.36 (br s, 1H), 5.16 (br s, 1H), 4.33 (br s, 1H), 4.16 (br dd, J=13.88, 7.64 Hz, 1H), 3.61-3.83 (m, 1H), 3.15 (br d, J=7.34 Hz, 2H), 2.99-3.07 (m, 2H), 2.90 (br d, J=7.21 Hz, 4H), 2.39 (dq, J=13.48, 6.75, 6.75, 6.75, 6.75 Hz, 1H), 2.01-2.23 (m, 4H), 1.81 (td, J=12.96, 6.36 Hz, 1H), 1.01 (d, J=6.72 Hz, 6H). LC-MS: m/z 628.7 (M+H)⁺.

### (R)-4-(7-((6,7-dihydro-5H-cyclopenta[b]pyridin-5-yl)amino)thieno[2,3-c]pyridin-2-yl)-2-isobutyl-5-(5-methyl-1,3,4-oxadiazol-2-yl)-6-(2-(tetrahydro-2H-pyran-4-yl)ethyl)nicotinamide (Compound 196)

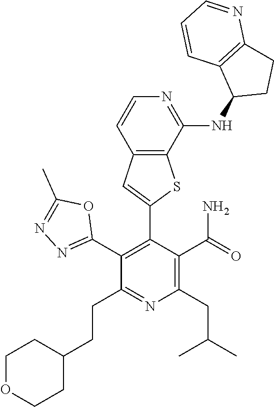 [SMILES: Cc1nnc(-c2c(CCC3CCOCC3)nc(CC(C)C)c(C(N)=O)c2-c2cc3ccnc(N[C@@H]4CCc5ncccc54)c3s2)o1]

Compound 196 was synthesized using a similar procedure described in the Example 10 above by using the appropriate materials.

¹H NMR (400 MHz, DMSO-d₆): δ 8.36 (d, J=4.8 Hz, 1H), 7.94 (d, J=5.2 Hz, 1H), 7.91 (s, 1H), 7.54-7.63 (m, 2H), 7.25-7.32 (m, 2H), 7.10-7.15 (m, 1H), 7.05 (d, J=5.6 Hz, 1H), 5.86 (d, J=7.6 Hz, 1H), 7.05 (dd, J=11.2 Hz, J=3.2 Hz, 2H), 3.20 (t, J=11.2 Hz, 2H), 2.98-3.08 (m, 1H), 2.86-2.97 (m, 1H), 2.74 (d, J=7.2 Hz, 2H), 2.35-2.72 (m, 2H), 2.53-2.58 (m, 1H), 2.38 (s, 3H), 2.26-2.35 (m, 1H), 2.00-2.12 (m, 1H), 1.36-1.60 (m, 5H), 1.01-1.16 (m, 2H), 0.95 (d, J=6.8 Hz, 6H). LC-MS: m/z 638.3 (M+H)⁺.

### 4-(7-((3,4-difluorobenzyl)amino)thieno[2,3-c]pyridin-2-yl)-2-isobutyl-5-(5-methyl-1,3,4-oxadiazol-2-yl)-6-(2-(tetrahydro-2H-pyran-4-yl)ethyl nicotinamide (Compound 197)

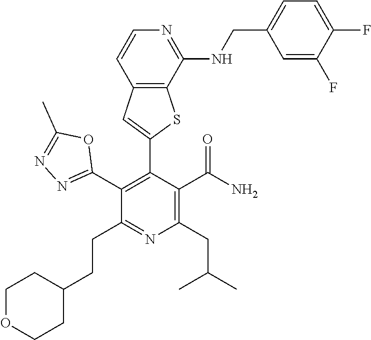 [SMILES: Cc1nnc(-c2c(CCC3CCOCC3)nc(CC(C)C)c(C(N)=O)c2-c2cc3ccnc(NCc4ccc(F)c(F)c4)c3s2)o1]

Compound 197 was synthesized using a similar procedure described in the Example 10 above by using the appropriate materials.

1H NMR (400 MHz, DMSO-d₆): δ 7.89-7.96 (m, 2H), 7.59 (br s, 1H), 7.27 (s, 1H), 7.18 (d, J=8.0 Hz, 1H), 7.17 (t, J=8.0 Hz, 1H), 7.02 (d, J=5.6 Hz, 1H), 6.81 (t, J=8.4 Hz, 2H), 5.87 (q, J=8.0 Hz, 1H), 3.75-3.83 (m, 5H), 3.15-3.25 (m, 2H), 2.89-2.98 (m, 1H), 2.64-2.77 (m, 5H), 2.38 (s, 3H), 2.23-2.36 (m, 2H), 1.94-2.04 (m, 1H), 1.37-1.60 (m, 5H), 1.02-1.16 (m, 2H), 0.95 (d, J=6.8 Hz, 6H). LC-MS: m/z 667.3 (M+H)⁺.

### (R)-2-isobutyl-4-(7-((4-methoxy-2,3-dihydro-1H-inden-1-yl)amino)thieno[2,3-c]pyridin-2-yl)-5-(5-methyloxazol-2-yl)-6-(2-(tetrahydro-2H-pyran-4-yl)ethyl)nicotinamide (Compound 198)

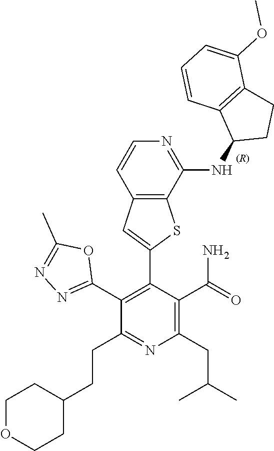

Compound 198 was synthesized using a similar procedure described in the Example 10 above by using the appropriate materials.

1H NMR (400 MHz, DMSO-d₆): δ 7.84-7.94 (m, 2H), 7.50-7.57 (m, 1H), 7.22 (s, 1H), 7.07-7.15 (m, 2H), 7.00 (d, J=5.6 Hz, 1H), 6.84-6.88 (m, 1H), 7.80 (t, J=8.4 Hz, 2H), 5.83-5.91 (m, 1H), 3.74-3.82 (m, 5H), 3.14-3.24 (m, 2H), 2.87-3.00 (m, 1H), 2.64-2.74 (m, 5H), 2.24-2.36 (m, 2H), 2.13 (s, 3H), 1.93-2.05 (m, 1H), 1.34-1.56 (m, 5H), 1.00-1.15 (m, 2H), 0.94 (d, J=6.8 Hz, 6H). LC-MS: m/z 666.4 (M+H)⁺.

### 6-(4-fluorophenethyl)-2-isobutyl-5-(5-methyl-1,3,4-oxadiazol-2-yl)-4-(7-((pyridin-2-ylmethyl)amino)thieno[2,3-c]pyridin-2-yl)nicotinamide (Compound 200)

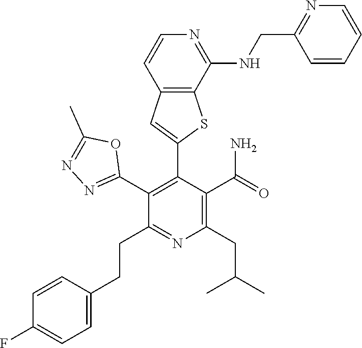 [SMILES: Cc1nnc(-c2c(CCc3ccc(F)cc3)nc(CC(C)C)c(C(N)=O)c2-c2cc3ccnc(NCc4ccccn4)c3s2)o1]

Compound 200 was synthesized using a similar procedure described in the Example 10 above by using the appropriate materials.

1H NMR (400 MHz, DMSO-d₆): δ 8.45-8.49 (m, 2H), 7.93 (s, 1H), 7.83 (d, J=5.2 Hz, 1H), 7.68 (t, J=7.6 Hz, 1H), 7.60 (s, 1H), 7.49 (t, J=5.6 Hz, 1H), 7.28 (s, 1H), 7.21-7.23 (m, 1H), 7.11-7.14 (m, 2H), 7.06 (t, J=8.8 Hz, 2H), 7.00 (d, J=5.2 Hz, 1H), 4.72 (d, J=6.0 Hz, 2H), 2.97-3.02 (m, 4H), 2.76 (d, J=7.2 Hz, 2H), 2.30-2.35 (m, 4H), 0.95 (d, J=6.8 Hz, 6H). ¹⁹F NMR (377 MHz, DMSO-d₆): −117.24. LC-MS: m/z 622.2 (M+H)⁺.

### 6-(4-fluorophenethyl)-2-isobutyl-5-(5-methyl-1,3,4-oxadiazol-2-yl)-4-(7-((pyridin-3-ylmethyl)amino)thieno[2,3-c]pyridin-2-yl)nicotinamide (Compound 201)

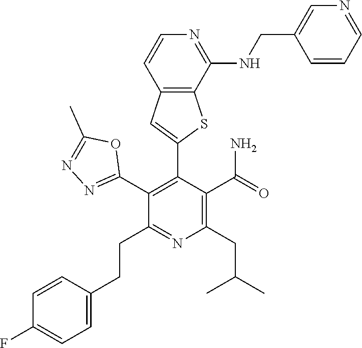 [SMILES: Cc1nnc(-c2c(CCc3ccc(F)cc3)nc(CC(C)C)c(C(N)=O)c2-c2cc3ccnc(NCc4cccnc4)c3s2)o1]

Compound 201 was synthesized using a similar procedure described in the Example 10 above by using the appropriate materials.

¹H NMR (400 MHz, DMSO-d₆): δ 8.54 (br s, 1H), 8.41 (d, J=4.0 Hz, 1H), 7.91 (br s, 1H), 7.87 (d, J=5.6 Hz, 1H), 7.70 (d, J=7.6 Hz, 1H), 7.58 (br s, 1H), 7.49 (t, J=5.6 Hz, 1H), 7.28-7.33 (m, 1H), 7.27 (s, 1H), 7.00-7.14 (m, 5H), 4.65 (d, J=5.6 Hz, 2H), 2.95-3.03 (m, 4H), 2.76 (d, J=7.2 Hz, 2H), 2.28-2.36 (m, 4H), 0.95 (d, J=6.4 Hz, 6H). ¹⁹F NMR (377 MHz, DMSO-d₆): δ −117.24. LC-MS: m/z 622.4 (M+H)⁺.

### 6-(4-fluorophenethyl)-2-isobutyl-5-(5-methyl-1,3,4-oxadiazol-2-yl)-4-(7-((pyridin-4-ylmethyl)amino)thieno[2,3-c]pyridin-2-yl)nicotinamide (Compound 202)

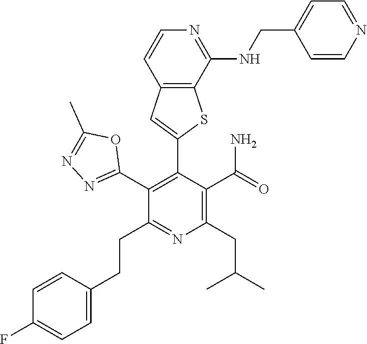 [SMILES: Cc1nnc(-c2c(CCc3ccc(F)cc3)nc(CC(C)C)c(C(N)=O)c2-c2cc3ccnc(NCc4ccncc4)c3s2)o1]

Compound 202 was synthesized using a similar procedure described in the Example 10 above by using the appropriate materials.

¹H NMR (400 MHz, DMSO-d₆): δ 8.47 (d, J=4.8 Hz, 2H), 7.94 (br s, 1H), 7.83 (d, J=5.6 Hz, 1H), 7.54-7.64 (m, 2H), 7.27-7.34 (m, 3H), 7.00-7.16 (m, 5H), 4.67 (d, J=5.6 Hz, 2H), 2.94-3.06 (m, 4H), 2.76 (d, J=7.2 Hz, 2H), 2.25-2.37 (m, 4H), 0.95 (d, J=6.8 Hz, 6H). ¹⁹F NMR (377 MHz, DMSO-d₆): −117.23. LC-MS: m/z 622.4 (M+H)⁺.

The following molecules were synthesized using a similar procedure described in the Examples above using the appropriate starting material.

| Compound | | LC-MS |
|---|---|---|
| No. | Structure | (m/z) |
| 155 | 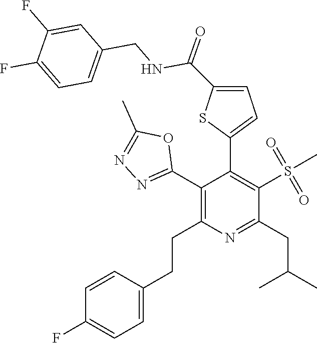 [SMILES: Cc1nnc(-c2c(CCc3ccc(F)cc3)nc(CC(C)C)c(S(C)(=O)=O)c2-c2ccc(C(=O)NCc3ccc(F)c(F)c3)s2)o1] | 669.1 (M + H)⁺ |
| 156 | 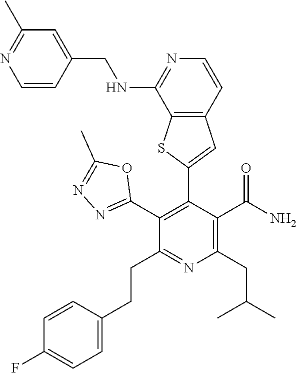 [SMILES: Cc1cc(CNc2nccc3cc(-c4c(C(N)=O)c(CC(C)C)nc(CCc5ccc(F)cc5)c4-c4nnc(C)o4)sc23)ccn1] | 636.2 (M + H)⁺ |
| 157 | 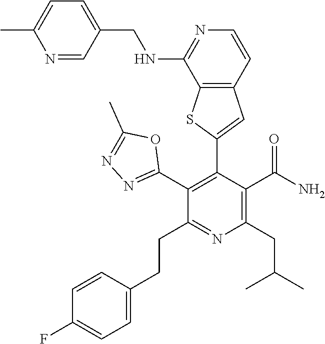 [SMILES: Cc1ccc(CNc2nccc3cc(-c4c(C(N)=O)c(CC(C)C)nc(CCc5ccc(F)cc5)c4-c4nnc(C)o4)sc23)cn1] | 636.2 (M + H)⁺ |
| 158 | 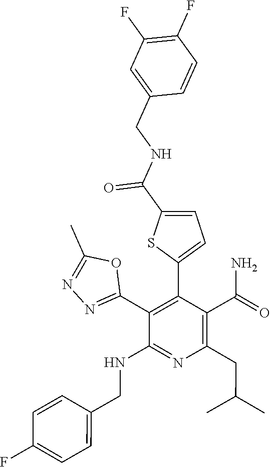 [SMILES: Cc1nnc(-c2c(NCc3ccc(F)cc3)nc(CC(C)C)c(C(N)=O)c2-c2ccc(C(=O)NCc3ccc(F)c(F)c3)s2)o1] | 635.4 (M + H)⁺ |
| 159 | 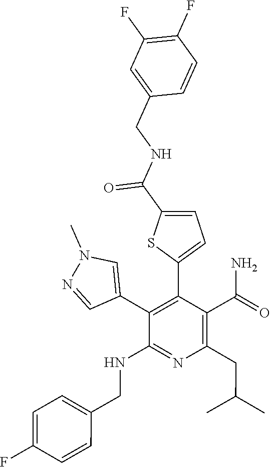 [SMILES: CC(C)Cc1nc(NCc2ccc(F)cc2)c(-c2cnn(C)c2)c(-c2ccc(C(=O)NCc3ccc(F)c(F)c3)s2)c1C(N)=O] | 632.2 (M + H)⁺ |
| 160 | 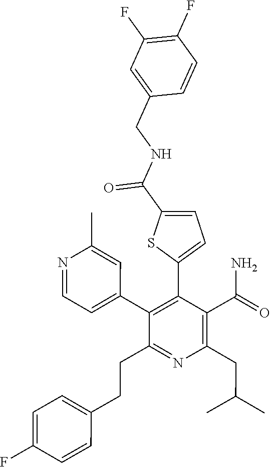 [SMILES: Cc1cc(-c2c(CCc3ccc(F)cc3)nc(CC(C)C)c(C(N)=O)c2-c2ccc(C(=O)NCc3ccc(F)c(F)c3)s2)ccn1] | 643.4 (M + H)⁺ |
| 161 | 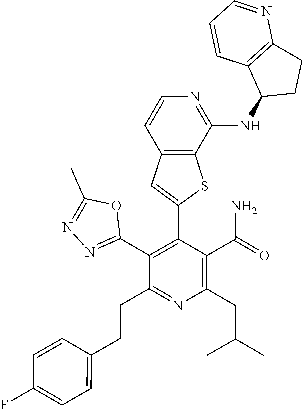 [SMILES: Cc1nnc(-c2c(CCc3ccc(F)cc3)nc(CC(C)C)c(C(N)=O)c2-c2cc3ccnc(N[C@@H]4CCc5ncccc54)c3s2)o1] | 648.3 (M + H)⁺ |
| 162 | 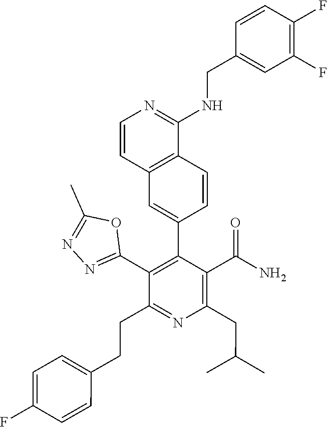 [SMILES: Cc1nnc(-c2c(CCc3ccc(F)cc3)nc(CC(C)C)c(C(N)=O)c2-c2ccc3c(NCc4ccc(F)c(F)c4)nccc3c2)o1] | 651.7 (M + H)⁺ |
| 163 | 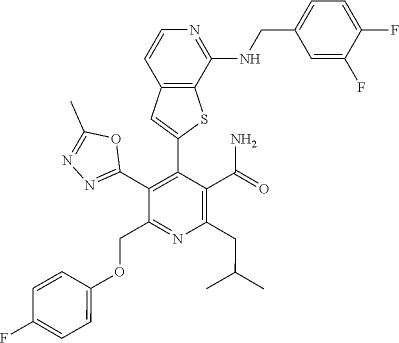 [SMILES: Cc1nnc(-c2c(COc3ccc(F)cc3)nc(CC(C)C)c(C(N)=O)c2-c2cc3ccnc(NCc4ccc(F)c(F)c4)c3s2)o1] | 659.4 (M + H)⁺ |
| 164 | 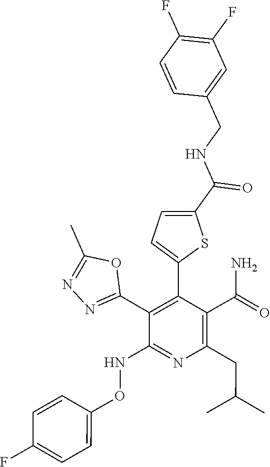 [SMILES: Cc1nnc(-c2c(NOc3ccc(F)cc3)nc(CC(C)C)c(C(N)=O)c2-c2ccc(C(=O)NCc3ccc(F)c(F)c3)s2)o1] | 636.2 (M + H)⁺ |
| 165 | 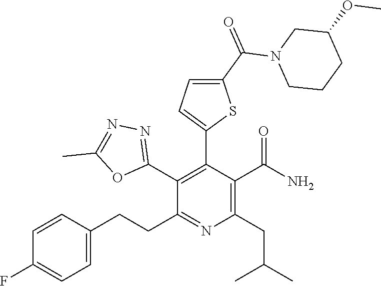 [SMILES: CO[C@@H]1CCCN(C(=O)c2ccc(-c3c(C(N)=O)c(CC(C)C)nc(CCc4ccc(F)cc4)c3-c3nnc(C)o3)s2)C1] | 606.4 (M + H)⁺ |
| 166 | 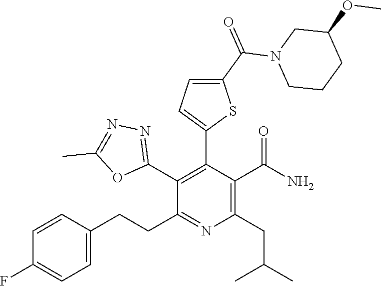 [SMILES: CO[C@H]1CCCN(C(=O)c2ccc(-c3c(C(N)=O)c(CC(C)C)nc(CCc4ccc(F)cc4)c3-c3nnc(C)o3)s2)C1] | 606.4 (M + H)⁺ |

### 6-[2-(p-fluorophenyl)ethyl]-4-(7-{[(5-fluoro-3-pyridyl)methyl]amino}-1-thia-6-aza-2-indenyl)-2-isobutyl-5-(5-methyl-1,3,4-oxadiazol-2-yl)nicotinamide (Compound 206)

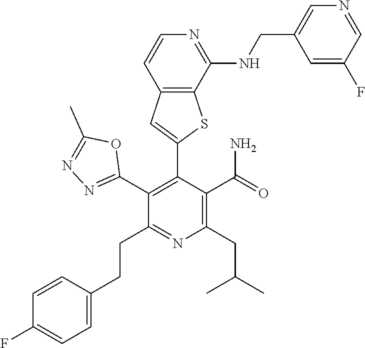 [SMILES: Cc1nnc(-c2c(CCc3ccc(F)cc3)nc(CC(C)C)c(C(N)=O)c2-c2cc3ccnc(NCc4cncc(F)c4)c3s2)o1]

Compound 206 was synthesized using similar procedure as described in Example 10 above by using the appropriate materials.

¹H NMR (400 MHz, DMSO-d6): δ 8.42-8.43 (m, 2H) 7.91 (s, 1H), 7.88 (d, J=5.6 Hz, 1H), 7.58-7.62 (m, 2H), 7.52 (t, J=6.0 Hz, 1H), 7.28 (s, 1H), 7.10-7.14 (m, 2H), 7.03-7.06 (m, 3H), 4.69 (d, J=6.0 Hz, 2H), 3.00-3.05 (m, 4H), 2.76 (d, J=7.2 Hz, 2H), 2.28-2.35 (m, 4H), 0.95 (d, J=6.8 Hz, 6H). ¹⁹F NMR (377 MHz, DMSO-d6): −117.24, −128.06. LC-MS: m/z 640.2 (M+H)⁺.

### 4-{7-[(R)-4-aza-1-indanylamino]-1-thia-6-aza-2-indenyl}-2-isobutyl-5-(5-methyl-1,3-oxazol-2-yl)-6-[2-(tetrahydro-2H-pyran-4-yl)ethyl]nicotinamide (Compound 207)

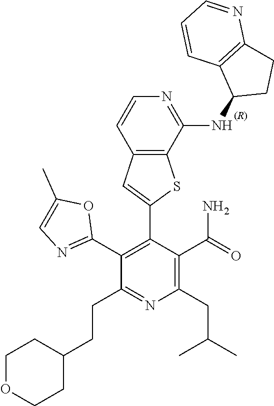

Compound 207 was synthesized using similar procedure as described in Example 25 above by using the appropriate materials.

¹H NMR (400 MHz, DMSO-d6): δ 8.36 (d, J=4.4 Hz, 1H), 7.93 (d, J=5.6 Hz, 1H), 7.87 (s, 1H), 7.50-7.60 (m, 2H), 7.20-7.27 (m, 2H), 7.09-7.16 (m, 1H), 7.03 (d, J=5.6 Hz, 1H), 6.86 (s, 1H), 5.80-5.91 (m, 1H), 3.71-3.83 (m, 2H), 3.12-3.24 (m, 2H), 2.83-2.97 (m, 1H), 2.63-2.75 (m, 5H), 2.22-2.35 (m, 2H), 2.13 (s, 3H), 2.00-2.10 (m, 1H), 1.32-1.57 (m, 5H), 1.00-1.14 (m, 2H), 0.94 (d, J=6.4 Hz, 6H). LC-MS: m/z 637.3 (M+H)⁺.

### Example 39: Synthesis of 4-{7-[(R)-4-methoxy-1-indanylamino]-1-thia-6-aza-2-indenyl}-2-isobutyl-5-(5-methyl-1,3,4-oxadiazol-2-yl)-6-[2-(tetrahydro-2H-pyran-4-yl)ethyl]nicotinamide (Compound 208)

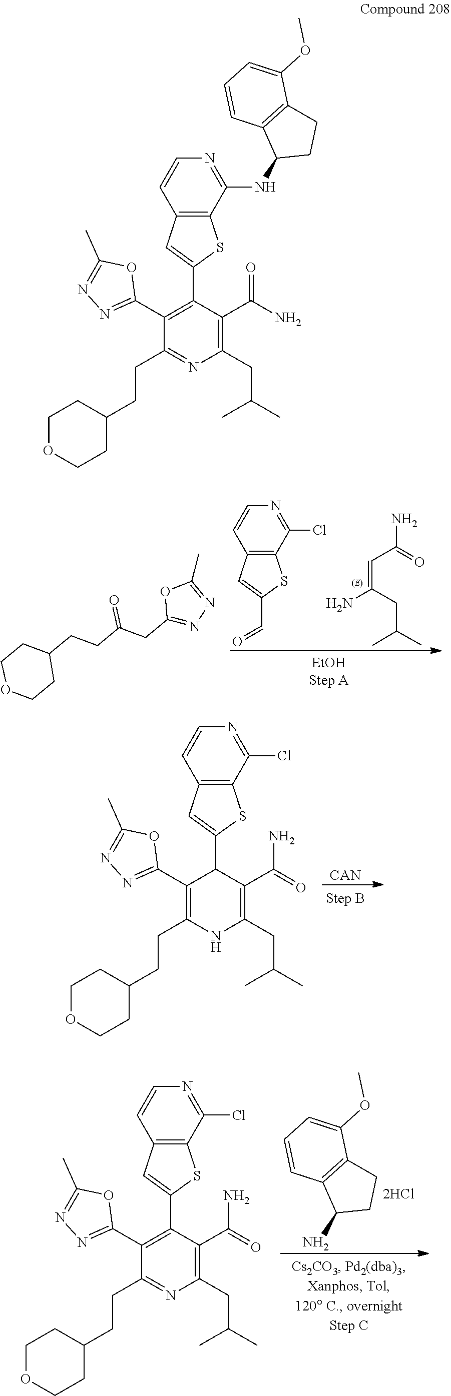

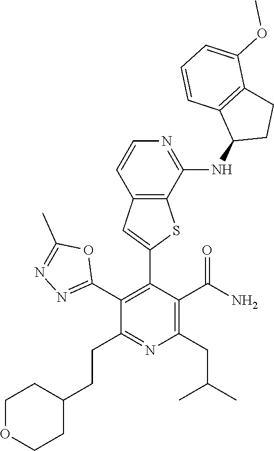

### Step A 4-(7-chlorothieno[2,3-c]pyridin-2-yl)-2-isobutyl-5-(5-methyl-1,3,4-oxadiazol-2-yl)-6-(2-(tetrahydro-2H-pyran-4-yl)ethyl)-1,4-dihydropyridine-3-carboxamide

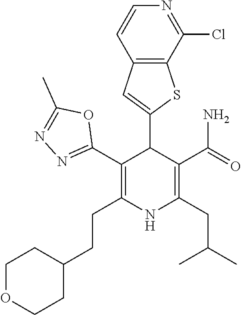 [SMILES: Cc1nnc(C2=C(CCC3CCOCC3)NC(CC(C)C)=C(C(N)=O)C2c2cc3ccnc(Cl)c3s2)o1]

To a solution of 1-(5-methyl-1,3,4-oxadiazol-2-yl)-4-(tetrahydro-2H-pyran-4-yl)butan-2-one (500 mg, 2.10 mmol) in EtOH (5 mL) were added 7-chlorothieno[2,3-c]pyridine-2-carbaldehyde (411.76 mg, 2.10 mmol) and (E)-3-amino-5-methylhex-2-enamide (298.00 mg, 2.10 mmol), and the reaction was stirred at 110° C. for 12 hours. The reaction was concentrated in vacuo to afford the crude 4-(7-chlorothieno[2,3-c]pyridin-2-yl)-2-isobutyl-5-(5-methyl-1,3,4-oxadiazol-2-yl)-6-(2-(tetrahydro-2H-pyran-4-yl)ethyl)-1,4-dihydropyridine-3-carboxamide (800 mg, crude) as yellow oil. LC-MS: m/z 542.3 (M+H)⁺

### Step B 4-(7-chlorothieno[2,3-c]pyridin-2-yl)-2-isobutyl-5-(5-methyl-1,3,4-oxadiazol-2-yl)-6-(2-(tetrahydro-2H-pyran-4-yl)ethyl)nicotinamide

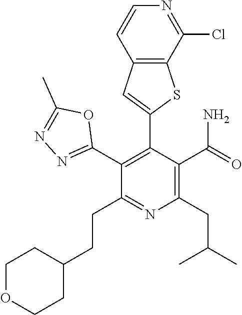 [SMILES: Cc1nnc(-c2c(CCC3CCOCC3)nc(CC(C)C)c(C(N)=O)c2-c2cc3ccnc(Cl)c3s2)o1]

A mixture of 4-(7-chlorothieno[2,3-c]pyridin-2-yl)-2-isobutyl-5-(5-methyl-1,3,4-oxadiazol-2-yl)-6-(2-(tetrahydro-2H-pyran-4-yl)ethyl)-1,4-dihydropyridine-3-carboxamide (800 mg, crude), diammonium cerium(IV) nitrate (1.62 g, 2.96 mmol) in DCM (10 mL) was stirred at rt for 2 hours. The mixture was diluted with EA (40 mL), washed with water (40 mL). The organic layer was dried over Na₂SO₄, filtered and concentrated under reduced pressure. The residue was purified by silica gel column chromatography, eluted with MeOH/DCM (1/20) to give 4-(7-chlorothieno[2,3-c]pyridin-2-yl)-2-isobutyl-5-(5-methyl-1,3,4-oxadiazol-2-yl)-6-(2-(tetrahydro-2H-pyran-4-yl)ethyl)nicotinamide (700 mg, yield: 87.83%) as a yellow solid. LC-MS: m/z 540.2 (M+H)⁺.

### Step C Compound 208

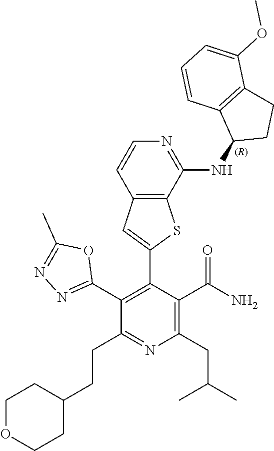

Compound 208 was synthesized using similar procedure as described in Example 10 above by using the appropriate materials.

1H NMR (400 MHz, DMSO-d6): δ 7.89-7.96 (m, 2H), 7.59 (br s, 1H), 7.27 (s, 1H), 7.18 (d, J=8.0 Hz, 1H), 7.17 (t, J=8.0 Hz, 1H), 7.02 (d, J=5.6 Hz, 1H), 6.81 (t, J=8.4 Hz, 2H), 5.87 (q, J=8.0 Hz, 1H), 3.75-3.83 (m, 5H), 3.15-3.25 (m, 2H), 2.89-2.98 (m, 1H), 2.64-2.77 (m, 5H), 2.38 (s, 3H), 2.23-2.36 (m, 2H), 1.94-2.04 (m, 1H), 1.37-1.60 (m, 5H), 1.02-1.16 (m, 2H), 0.95 (d, J=6.8 Hz, 6H). LC-MS: m/z 667.3 (M+H)⁺.

### Example 40: 4-{7-[(R)-6-fluoro-1-indanylamino]-1-thia-6-aza-2-indenyl}-6-ethyl-2-isobutyl-5-(5-methyl-1,3,4-oxadiazol-2-yl)nicotinamide (Compound 209)

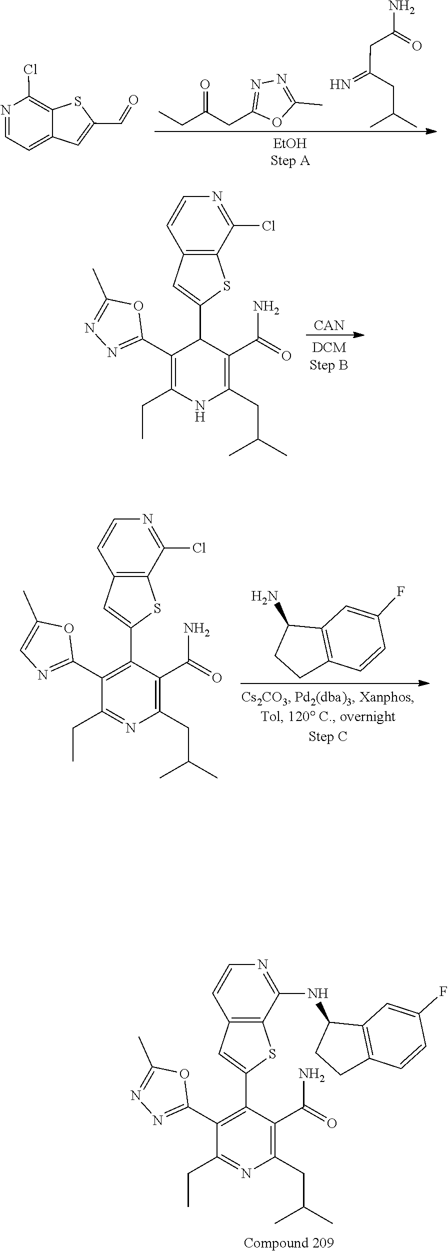

### Step A 4-(7-chlorothieno[2,3-c]pyridin-2-yl)-6-ethyl-2-isobutyl-5-(5-methyl-1,3,4-oxadiazol-2-yl)-1,4-dihydropyridine-3-carboxamide

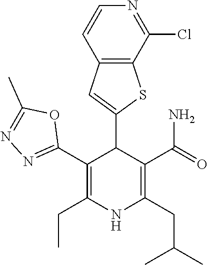 [SMILES: CCC1=C(c2nnc(C)o2)C(c2cc3ccnc(Cl)c3s2)C(C(N)=O)=C(CC(C)C)N1]

To a solution of 7-chlorothieno[2,3-c]pyridine-2-carbaldehyde (500 mg, 2.54 mmol) in EtOH (5 mL) were added 1-(5-methyl-1,3,4-oxadiazol-2-yl)butan-2-one (390.86 mg, 2.54 mmol) and 3-imino-5-methylhexanamide (288.32 mg, 2.03 mmol), and the reaction was stirred at 110° C. for 12 hours. The reaction was concentrated in vacuo to afford the crude 4-(7-chlorothieno[2,3-c]pyridin-2-yl)-6-ethyl-2-isobutyl-5-(5-methyl-1,3,4-oxadiazol-2-yl)-1,4-dihydropyridine-3-carboxamide (1.17 g, crude) as a yellow oil. LC-MS: m/z 458.2 (M+H)⁺.

### Step B The synthesis of 4-(7-chlorothieno[2,3-c]pyridin-2-yl)-6-ethyl-2-isobutyl-5-(5-methyl-1,3,4-oxadiazol-2-yl)nicotinamide

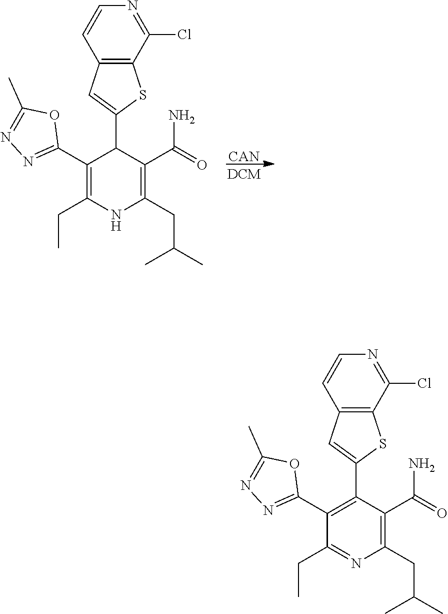

A mixture of 4-(7-chlorothieno[2,3-c]pyridin-2-yl)-6-ethyl-2-isobutyl-5-(5-methyl-1,3,4-oxadiazol-2-yl)-1,4-dihydropyridine-3-carboxamide (1.17 g, crude), diammonium cerium(IV) nitrate (2.81 g, 5.12 mmol) in DCM (12 mL) was stirred at room temperature for 2 hours. The mixture was diluted with EA (40 mL), washed with water (40 mL). The organic layer was dried over Na₂SO₄, filtered and concentrated under reduced pressure. The residue was purified by silica gel column chromatography, eluted with MeOH/DCM (1/20) to give 4-(7-chlorothieno[2,3-c]pyridin-2-yl)-6-ethyl-2-isobutyl-5-(5-methyl-1,3,4-oxadiazol-2-yl)nicotinamide (674 mg, yield: 57.86%) as a yellow solid. LC-MS: m/z 456.2 (M+H)⁺.

### Step C Synthesis of Compound 209

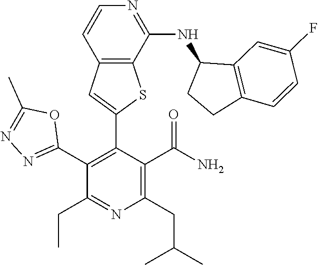 [SMILES: CCc1nc(CC(C)C)c(C(N)=O)c(-c2cc3ccnc(N[C@@H]4CCc5ccc(F)cc54)c3s2)c1-c1nnc(C)o1]

Compound 209 was synthesized using similar procedure as described in Example 10 above by using the appropriate materials.

¹H NMR (400 MHz, DMSO-d6): δ 7.95 (d, J=5.6 Hz, 1H), 7.91 (br s, 1H), 7.59 (br s, 1H), 7.25-7.30 (m, 3H), 6.98-7.07 (m, 2H), 6.90-6.96 (m, 1H), 5.84 (dd, J=6.8 Hz, J=15.2 Hz, 1H), 2.91-3.00 (m, 1H), 2.73-2.85 (m, 3H), 2.67 (q, J=7.2 Hz, 2H), 2.38 (s, 3H), 2.27-2.35 (m, 2H), 2.00-2.11 (m, 1H), 1.19 (t, J=7.6 Hz, 3H), 0.96 (d, J=6.4 Hz, 6H). ¹⁹F NMR (377 MHz, DMSO-d6): −117.35. LC-MS: m/z 571.3 (M+H)⁺.

### Example 41: N—[(R)-1-indanyl]-5-{3-carbamoyl-6-[(p-fluorophenoxy)methyl]-2-isobutyl-5-(5-methyl-1,3,4-oxadiazol-2-yl)-4-pyridyl}-2-thenamide (Compound 210)

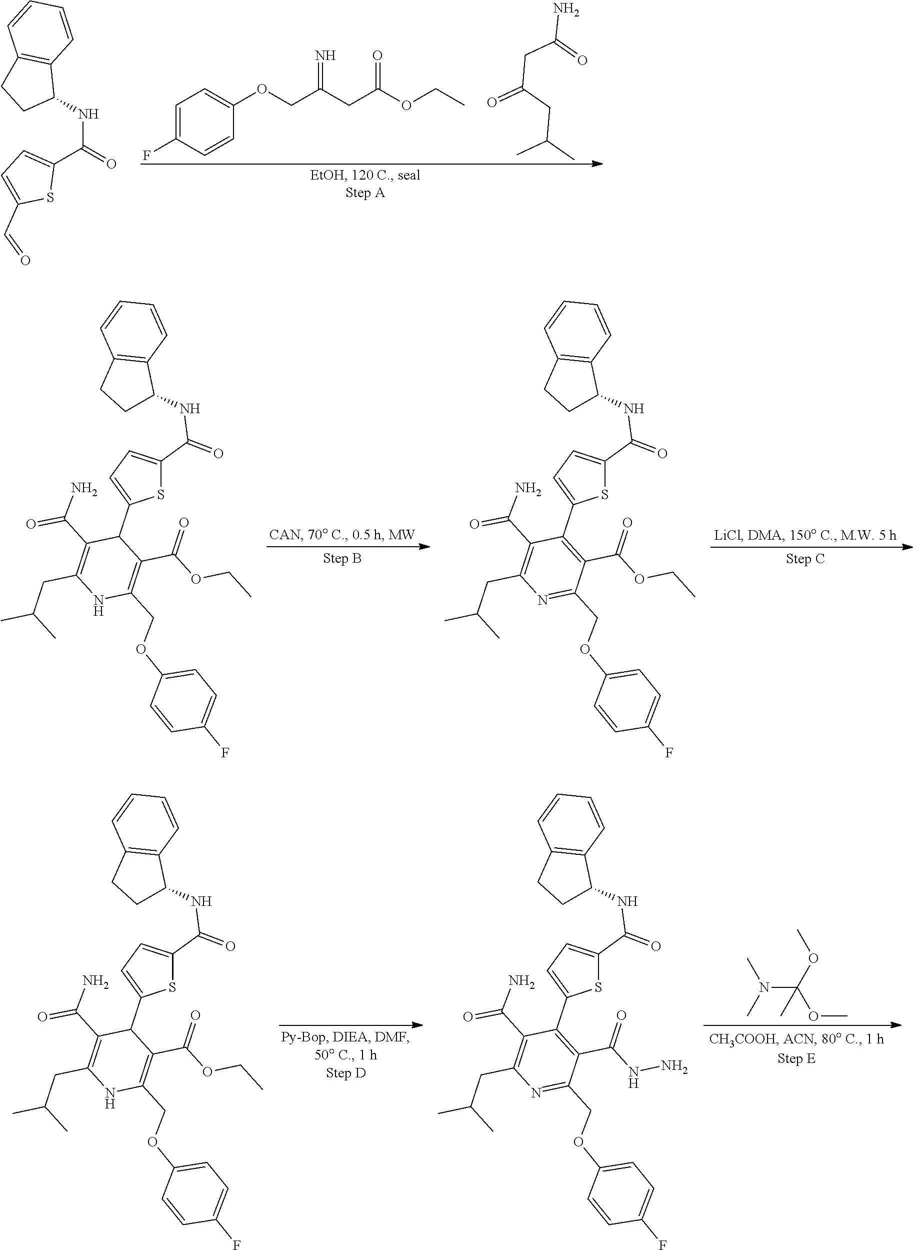

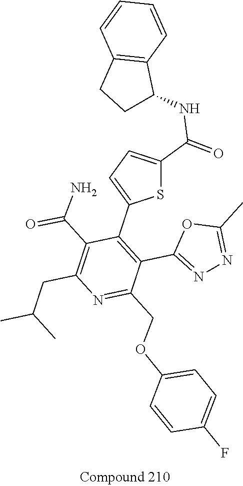 [SMILES: Cc1nnc(-c2c(COc3ccc(F)cc3)nc(CC(C)C)c(C(N)=O)c2-c2ccc(C(=O)N[C@@H]3CCc4ccccc43)s2)o1]

### Step A ethyl 5-carbamoyl-4-(5-(((R)-2,3-dihydro-1H-inden-1-yl)carbamoyl)thiophen-2-yl)-2-((4-fluorophenoxy)methyl)-6-isobutyl-1,4-dihydropyridine-3-carboxylate

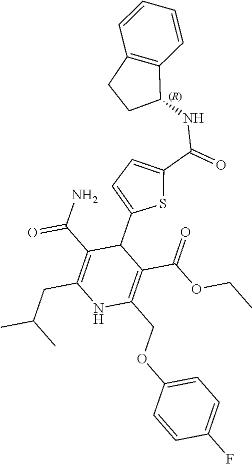

To a solution of N-[(1R)-2,3-dihydro-1H-indenyl]-5-formylthiophene-2-carboxamide (370 mg, 1.36 mmol) in EtOH (12 mL) were added ethyl 3-azanylidene-4-[(4-fluorophenyl)oxy]butanoate (326 mg, 1.36 mmol), 5-methyl-3-oxohexanamide (195 mg, 1.36 mmol) and the reaction was stirred at 120° C. for overnight with N2 protection in a seal tube. The residue was purified using TLC with EtOAc:PE=1.5:1 and concentrated in vacuum to afford the compound ethyl 5-carbamoyl-2-{[(4-fluorophenyl)oxy]methyl}-4-[5-({[(1R)-2,3-dihydro-1H-indenyl]amino}carbonyl)thiophen-2-yl]-6-(2-methylpropyl)-1,4-dihydropyridine-3-carboxylate (230 mg, yield: 27.30%) as a yellow solid. LC-MS: m/z 618.2 (M+H)⁺.

### Step B ethyl (R)-5-carbamoyl-4-(5-((2,3-dihydro-1H-inden-1-yl)carbamoyl)thiophen-2-yl)-2-((4-fluorophenoxy)methyl)-6-isobutylnicotinate

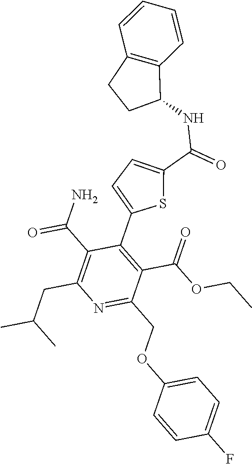 [SMILES: CCOC(=O)c1c(COc2ccc(F)cc2)nc(CC(C)C)c(C(N)=O)c1-c1ccc(C(=O)N[C@@H]2CCc3ccccc32)s1]

To a solution of ethyl 5-carbamoyl-2-{[(4-fluorophenyl)oxy]methyl}-4-[5-({[(1R)-2,3-dihydro-1H-indenyl]amino}carbonyl)thiophen-2-yl]-6-(2-methylpropyl)-1,4-dihydropyridine-3-carboxylate (230 mg, 0.37 mmol) in DCM (12 mL) were added Ceric ammonium nitrate (612 mg, 1.12 mmol) and the reaction was stirred at 70° C. for 30 min in microwave. The reaction was filtered and concentrated in vacuum. The residue was purified using silica gel column chromatography eluting with PE:EA=1:1 and concentrated in vacuum to afford the compound ethyl 5-carbamoyl-2-{[(4-fluorophenyl)oxy]methyl}-4-[5-({[(1R)-2,3-dihydro-1H-indenyl]amino}carbonyl)thiophen-2-yl]-6-(2-methylpropyl)pyridine-3-carboxylate (90 mg, yield: 39.26%) as a yellow solid. LC-MS: m/z 616.1 (M+H)⁺.

### Step C (R)-5-carbamoyl-4-(5-((2,3-dihydro-1H-inden-1-yl)carbamoyl)thiophen-2-yl)-2-((4-fluorophenoxy)methyl)-6-isobutylnicotinic acid

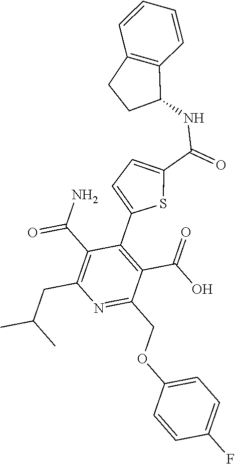 [SMILES: CC(C)Cc1nc(COc2ccc(F)cc2)c(C(=O)O)c(-c2ccc(C(=O)N[C@@H]3CCc4ccccc43)s2)c1C(N)=O]

To a solution of ethyl 5-carbamoyl-2-{[(4-fluorophenyl)oxy]methyl}-4-[5-({[(1R)-2,3-dihydro-1H-indenyl]amino}carbonyl)thiophen-2-yl]-6-(2-methylpropyl)pyridine-3-carboxylate (90 mg, 0.15 mmol) in DMA (5 mL) were added LiCl (93 mg, 2.19 mmol) and the reaction was stirred at 150° C. for 3 hours in microwave with N₂ protection. The reaction was diluted with EtOAc and brine. The organic layer was separated, washed with further brine, and concentrated in vacuum. The residue was purified using reversed phase column with 0.1% FA to afford the compound 5-carbamoyl-2-{[(4-fluorophenyl)oxy]methyl}-4-[5-({[(1R)-2,3-dihydro-1H-indenyl]amino}carbonyl)thiophen-2-yl]-6-(2-methylpropyl)pyridine-3-carboxylic acid (20 mg, yield: 23.28%) as a white solid. LC-MS: m/z 588.2 (M+H)⁺.

### Step D (R)-4-(5-((2,3-dihydro-1H-inden-1-yl)carbamoyl)thiophen-2-yl)-6-((4-fluorophenoxy)methyl)-5-(hydrazinecarbonyl)-2-isobutylnicotinamide

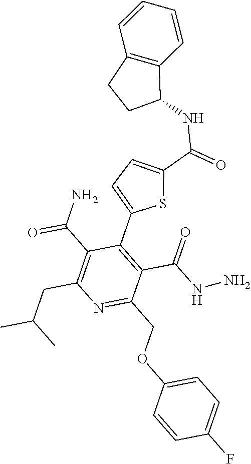 [SMILES: CC(C)Cc1nc(COc2ccc(F)cc2)c(C(=O)NN)c(-c2ccc(C(=O)N[C@@H]3CCc4ccccc43)s2)c1C(N)=O]

To a solution of 5-carbamoyl-2-{[(4-fluorophenyl)oxy]methyl}-4-[5-({[(1R)-2,3-dihydro-1H-indenyl]amino}carbonyl)thiophen-2-yl]-6-(2-methylpropyl)pyridine-3-carboxylic acid (20 mg, 0.034 mmol) in DMF (1 mL) were added Hydrazine monohydrochloride (5 mg, 0.07 mmol), Benzotriazole-1-yl-oxytripyrrolidinophosphonium hexafluorophosphate (27 mg, 0.05 mmol), and DIEA (18 mg, 0.14 mmol), and the reaction was stirred at 50° C. for 1 hour. The reaction was diluted with EtOAc and brine. The organic layer was separated, washed with further brine, and concentrated in vacuum. The residue was purified using reversed phase column with 0.1% FA to afford the compound 5-(diazanylcarbonyl)-6-{[(4-fluorophenyl)oxy]methyl}-4-[5-({[(1R)-2,3-dihydro-1H-indenyl]amino}carbonyl)thiophen-2-yl]-2-(2-methylpropyl)pyridine-3-carboxamide (15 mg, yield: 73.24%) as a white solid. LC-MS: m/z 602.3 (M+H)⁺.

### Step E N—[(R)-1-indanyl]-5-{3-carbamoyl-6-[(p-fluorophenoxy)methyl]-2-isobutyl-5-(5-methyl-1,3,4-oxadiazol-2-yl)-4-pyridyl}-2-thenamide

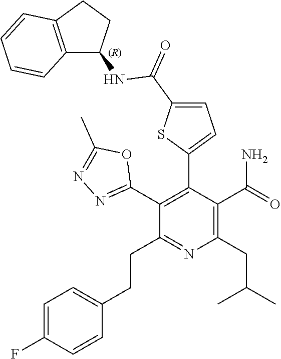

To a solution of 5-(diazanylcarbonyl)-6-{[(4-fluorophenyl)oxy]methyl}-4-[5-({[(1R)-2,3-dihydro-1H-indenyl]amino}carbonyl)thiophen-2-yl]-2-(2-methylpropyl)pyridine-3-carboxamide (15 mg, 0.025 mmol) in ACN (2 mL) were added 1-(dimethylamino)-1,1-dimethoxyethane (4 mg, 0.027 mmol) and the reaction was stirred at 80° C. for 1 hour. After 1 hour was added Acetic acid (2 mL) and the reaction was stirred at 80° C. for 1 hour. The reaction was diluted with EtOAc and brine. The organic layer was separated, washed with further brine, and concentrated in vacuum. The residue was purified by Pre-HPLC with 0.1% FA to afford the compound 6-{[(4-fluorophenyl)oxy]methyl}-4-[5-({[(1R)-2,3-dihydro-1H-indenyl]amino}carbonyl)thiophen-2-yl]-5-(5-methyl-1,3,4-oxadiazol-2-yl)-2-(2-methylpropyl)pyridine-3-carboxamide (1.46 mg, yield: 9.36%) as a white solid.

¹H NMR (400 MHz, Methanol-d4): δ 7.59 (d, J=3.6 Hz, 1H), 7.18-7.26 (m, 4H), 7.05 (d, J=3.6 Hz, 1H), 6.94 (t, J=9.2 Hz, 2H), 6.74-6.78 (m, 2H), 5.56 (t, J=7.2 Hz, 1H), 5.30 (m, 2H), 3.02-3.09 (m, 1H), 2.89-2.93 (m, 1H), 2.86 (d, J=7.2 Hz, 2H), 2.51-2.58 (m, 1H), 2.40 (s, 3H), 1.96-2.03 (m, 1H), 1.29-1.33 (m, 2H), 1.00 (d, J=6.8 Hz, 6H). 19F NMR (Methanol-d4): δ −125.24. LC-MS: m/z 626.2 (M+H)⁺

### Biological Assays

Amylin Receptor cAMP Assay I

AMYRs are heterodimers of the class B calcitonin (CT) G-protein-coupled receptor (CTR) and receptor activity-modifying proteins (RAMPs). All three RAMPs can interact with the CTR and form AMY1, AMY2, AMY3 with RAMP1, RAMP2 and RAMP3, respectively. Like other class B1 GPCRs, the CT receptor family is canonically coupled to Gs-mediated cAMP production, and measurement of cAMP accumulation has been the primary assay used to determine peptide selectivity and potency.

To optimize functional activity directed toward Gas coupling, COS-7 cells were stably transfected with human calcitonin receptor (CTR) and RAMP3, simultaneously. 100× concentration of compound working solutions were prepared (Agilent Technologies Bravo) with 4-fold serial dilution in 384-well Echo LDV plate (Labcyte, Cat #LP-0200). 100 nL/well 100× concentration of compound working solutions were moved to 384-well white low volume plate (Greiner, Cat #784075) using Labcyte ECHO550. 1×105 cells/mL COS-7/CTR or COS-7/AMY3 (HD Biosciences) cell suspensions prepared with assay buffer [DPBS containing 0.5 mM IBMX (Sigma, Cat #15879) and 0.1% BSA (GENVIEW, Cat #FA016-100 g)], 10 μL cell suspensions were added to each well of previous generated assay plate which already contains 100 nL compound at 100× concentration using ThermoFisher Multidrop Combi (1000 cells/well). Seal the plate and incubate at 37° C. with 5% CO₂ for 30 min.

After incubation, the cAMP assay signal was generated using cAMP Hi-range Kit (Cisbio). 5 μL cAMP-d2 working solution was added to each well, followed by 5 μL Anti-cAMP antibody-cryptate working solution which was added to each well using ThermoFisher Multidrop Combi. The samples were then incubated at room temperature for 1 hour protected from light. the fluorescence was read at 665 and 615 nm with Reader PerkinElmer EnVision.

$$\% Activity = 100 \% \times ( mean RLU of test sample - mean RLU of vehicle control ) / \text{} ( mean RLU of MAX control - mean RLU of vehicle control ) )$$

Amylin Receptor cAMP Assay II

AMYRs are heterodimers of the class B calcitonin (CT) G-protein-coupled receptor (CTR) and receptor activity-modifying proteins (RAMPs). All three RAMPs can interact with the CTR and form AMY1, AMY2, AMY3 with RAMP1, RAMP2 and RAMP3, respectively. Like other class B1 GPCRs, the CT receptor family is canonically coupled to Gs-mediated cAMP production, and measurement of cAMP accumulation has been the primary assay used to determine peptide selectivity and potency.

To optimize functional activity directed toward Gas coupling, COS-7 cells were stably transfected with human calcitonin receptor (CTR) and RAMP3, simultaneously. 100× concentration of compound working solutions were prepared with 4-fold serial dilution in 384-well Echo LDV plate (Labcyte, Cat #LP-0200-BC). 200 nL/well 100× concentration of compound working solutions were moved to 384-well white microplate (Perkin Elmer, Cat #6007680) using Labcyte ECHO550. 1×10⁵ cells/mL COS-7/AMY3 cell suspensions prepared with assay buffer [HBSS containing 20 mM HEPES (Gibco, Cat #15630-080), 0.5 mM IBMX (Sigma, Cat #15879) and 0.1% Casein (Sigma, Cat #C4765)], 20 μL cell suspensions were added to each well of previous generated assay plate which already contains 200 nL compound at 100× concentration using ThermoFisher Multidrop Combi (2000 cells/well). Seal the plate and incubate at 37° C. with 5% CO₂ for 30 min.

After incubation, the cAMP assay signal was generated using cAMP dynamic 2 kit (Revvity, Cat #62AM4PEC). 10 μL cAMP-d2 working solution was added to each well, followed by 10 μL Anti-cAMP antibody-cryptate working solution which was added to each well using CERTUS FLEX LIQUID DISPENSER. The samples were then incubated at room temperature for 1 hour protected from light. the fluorescence was read at 665 and 615 nm with Reader PerkinElmer EnVision 2105.

$$\% Activity = 100 - ( mean RLU of test sample - mean RLU of positive control ) / \text{} ( mean RLU of vehicle control - mean RLU of positive control ) \times 100$$

Calcitonin Receptor (CTR) cAMP Assay

The calcitonin receptor (CTR) belongs to the subfamily of GPCRs known as the secretin or ‘B’ family of GPCRs. Like other class B1 GPCRs, the CT receptor family is canonically coupled to Gs-mediated cAMP production, and measurement of cAMP accumulation has been the primary assay used to determine peptide selectivity and potency.

To optimize functional activity directed toward Gas coupling, COS-7 cells were stably transfected with human calcitonin receptor (CTR) to create COS7-human CTR Clone #2 stable cell line. 100× concentration of compound working solutions were prepared with 4-fold serial dilution in 384-well Echo LDV plate (Labcyte, Cat #LP-0200-BC). 200 nL/well 100× concentration of compound working solutions were moved to 384-well white microplate (Perkin Elmer, Cat #6007680) using Labcyte ECHO550. 1×10⁵ cells/mL COS7-human CTR Clone #2 cell suspensions prepared with assay buffer [HBSS containing 20 mM HEPES (Gibco, Cat #15630-080), 0.5 mM IBMX (Sigma, Cat #15879) and 0.1% Casein (Sigma, Cat #C4765)], 20 μL cell suspensions were added to each well of previous generated assay plate which already contains 200 nL compound at 100× concentration using ThermoFisher Multidrop Combi (2000 cells/well). Seal the plate and incubate at 37° C. with 5% CO₂ for 30 min.

After incubation, the cAMP assay signal was generated using cAMP dynamic 2 kit (Revvity, Cat #62AM4PEC). 10 μL cAMP-d2 working solution was added to each well, followed by 10 L Anti-cAMP antibody-cryptate working solution which was added to each well using CERTUS FLEX LIQUID DISPENSER. The samples were then incubated at room temperature for 1 hour protected from light. the fluorescence was read at 665 and 615 nm with Reader PerkinElmer EnVision 2105.

$$\% Activity = 100 - ( mean RLU of test sample - mean RLU of positive control ) / \text{} ( mean RLU of vehicle control - mean RLU of positive control ) \times 100$$

### Computational Assay

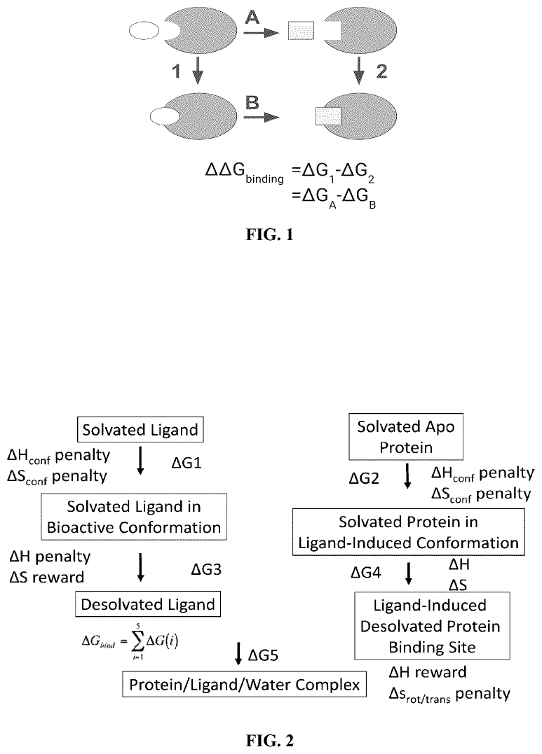

The binding potency of a ligand to a protein receptor can be determined using Free Energy Perturbation (FEP), a physics-based free energy perturbation technology for computationally predicting protein-ligand binding potency (Schrödinger Inc., see, e.g.,^{1,2} as depicted in FIG. 1 and FIG. 2).

The results provided in the Table and Figures were generated using FEP+ with the OPLS4 force field in Life Science Schrödinger Suite Release 2023,^{1,3} using sampling timescale of 15 ns. Relative FEP calculations are run with reference to congeneric ligands with previously measured experimental in vitro potency against the human calcitonin receptor or human amylin receptor. The binding mode of the congeneric ligands are defined on the basis of a resolved ligand protein complex structure, here a cryogenic electron microscopy (cryo-EM) structure, and the whole system can be described in full atomistic detail to include ligand, protein, lipid bilayer and water molecule(s).

The mean unsigned error between the FEP+ with OPLS4 values and the human calcitonin receptor agonist cAMP experimental measurements was calculated to be 0.8 log units (˜1.1 kcal/mol), which is in line with the average performance across drug discovery projects, as referenced in 2. The mean unsigned error between the FEP predicted values and the human amylin receptor agonist cAMP experimental measurements was calculated to be 0.6 log units.

### REFERENCES

1. Wang, L. et al. Accurate and reliable prediction of relative ligand binding potency in prospective drug discovery by way of a modern free-energy calculation protocol and force field. J. Am. Chem. Soc. 137, 2695-2703 (2015).
2. Abel, R., et al. A Critical Review of Validation, Blind Testing, and Real-World Use of Alchemical Protein-Ligand Binding Free Energy Calculations. Curr. Top. Med. Chem. 17, 2577-2585 (2017), 2577-2585 (2017).
3. Lu, C. et al. OPLS4: Improving Force Field Accuracy on Challenging Regimes of Chemical Space. J Chem. Theory Comput. 17, 4291-4300 (2021).

The activity of the tested compounds is provided in Table 3 below. In the Computational Assay, the input was the compound structure as shown in Table 1. In the in vitro assays, the tested compounds were prepared according to the examples described herein.

TABLE 3

| | Amylin Receptor | Calcitonin Receptor |
|---|---|---|
| Compound | cAMP Stimulation | cAMP Stimulation |
| No. | Activity: EC₅₀ (nM) | Activity: EC₅₀ (nM) |
| 101 | 84.7* | |
| 102 | 85.9* | |
| 103 | 314* | |
| 104 | 444* | |
| 105 | 591* | |
| 106 | 4.1* | 2.1 |
| 107 | 6.6* | 1.6 |
| 108 | 70.7* | |
| 109 | 20* | |
| 110 | 74.1* | |
| 111 | 650* | |
| 112 | >11400* | |
| 113 | 6.2* | |
| 114 | 323* | |
| 115 | 85* | |
| 116 | 13.9* | |
| 117 | 40.2* | |
| 118 | 211* | |
| 119 | 2.7* | |
| 120 | 1.7* | |
| 121 | 1.0* | |
| 122 | 0.8* | 0.73 |
| 123 | 37.2* | |
| 124 | 35.8* | |
| 125 | 212* | |
| 126 | 162* | |
| 127 | 495* | |
| 128 | 162* | |
| 129 | 940* | |
| 130 | 49* | |
| 131 | 4.4* | 0.33 |
| 132 | 2.5* | |
| 133 | 6.9* | |
| 134 | 1160* | |
| 135 | >1000* | 280 |
| 136 | 6.7* | |
| 137 | 1.3* | |
| 138 | 2260* | |
| 139 | 9930* | |
| 140 | 177* | |
| 141 | 50* | |
| 142 | 94* | 8.8 |
| 143 | 374* | |
| 144 | 51.4* | 6.9 |
| 145 | 6290* | |
| 146 | 973* | |
| 147 | 2.3* | |
| 148 | 208* | |
| 149 | 19.1* | |
| 150 | 3.5* | |
| 151 | 134* | |
| 152 | 11.5* | |
| 153 | 12* | |
| 154 | 68.7* | |
| 155 | >1000* | |
| 156 | 1200* | |
| 157 | 625* | |
| 158 | 120* | |
| 159 | 91* | |
| 160 | 26* | |
| 161 | 13* | |
| 162 | 74* | |
| 163 | 40* | |
| 164 | 325* | |
| 165 | >1000* | |
| 166 | >1000* | |
| 167 | 336* | |
| 168 | 540* | |
| 169 | 122* | |
| 170 | 3720* | |
| 171 | 59.7* | |
| 172 | 1140* | |
| 173 | 408* | |
| 174 | 107* | 5.7 |
| 175 | 211* | |
| 176 | 1280* | |
| 177 | 394* | |
| 178 | 112* | |
| 179 | >1000* | |
| 180 | 138* | |
| 181 | 9320* | |
| 182 | 23300* | |
| 183 | 1910* | |
| 184 | 1070* | |
| 185 | 507* | |
| 186 | 123* | |
| 187 | 99.3* | |
| 188 | 36.6* | 8.1 |
| 189 | >1000* | |
| 190 | 4060* | 738 |
| 191 | 1080* | |
| 192 | >1000* | |
| 193 | 3550* | |
| 194 | 3400* | |
| 195 | 39900* | |
| 196 | 279* | |
| 197 | 13* | |
| 198 | 229* | |
| 199 | >1000* | |
| 200 | 273* | |
| 201 | 381* | |
| 202 | 242* | |
| 203 | 939* | 65 |
| 204 | 67.3* | |
| 205 | 66** | 6.0 |
| 206 | 41.5 | |
| 207 | 64.3 | |
| 208 | 18.9 | 0.581 |
| 209 | 96.9 | |
| 210 | 546 | |
| 211 | 7.25^{†} | 9.17^{†} |
| 212 | 8.29^{†} | 10.2^{†} |
| 213 | 7.75^{†} | 9.66^{†} |
| 214 | 6.08^{†} | 8^{†} |
| 215 | 6.66^{†} | 8.57^{†} |

*Amylin Receptor cAMP Assay I
**Amylin Receptor cAMP Assay II
^{†}Computational Assay. Data here is pEC₅₀, the log value of EC₅₀.

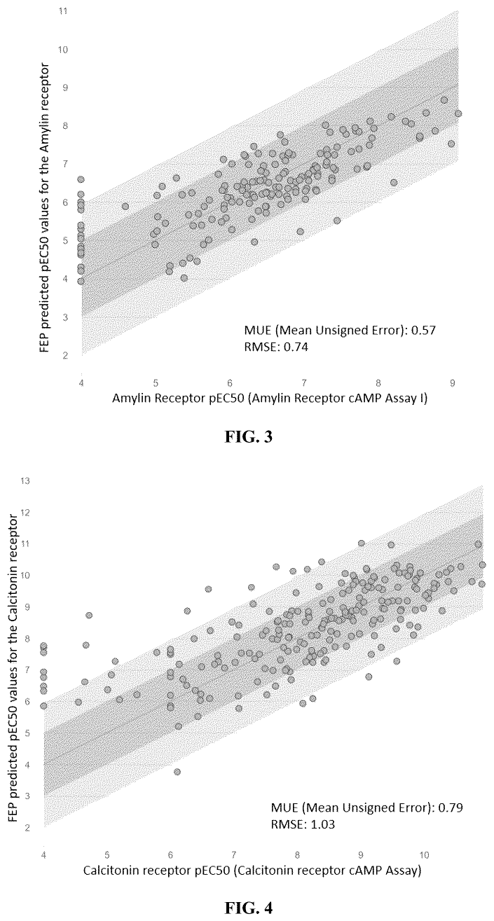

FIG. 3 illustrates the association between the in vitro Amylin Receptor cAMP Assay I and the in silico Computational Assay for representative compounds described herein and other compounds with known activity in the respective assays as described.

FIG. 4 illustrates the association between the in vitro Calcitonin receptor (CTR) cAMP Assay and the in silico Computational Assay for representative compounds described herein and other compounds with known activity in the respective assays as described.

FIGS. 3 and 4 show that the Computational Assay could be an accurate predictor for in vitro Amylin and Calcitonin receptor activity.

It is contemplated that the compounds of this disclosure are dual amylin and calcitonin receptor agonists (DACRAs), based on hCTR activity and the observed correlation with hAMYR3 activity.

1. A compound of Formula I: 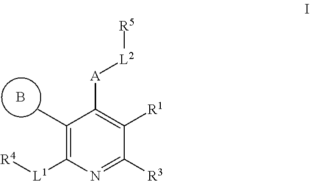
or a pharmaceutically acceptable salt, stereoisomer, mixture of stereoisomers, or solvate thereof, wherein:
A is C₁₋₆ alkylene, C₂₋₆ alkenylene, C₂₋₆ alkynylene, C₃₋₁₀ cycloalkylene, heterocyclylene, arylene, or heteroarylene; wherein the C₁₋₆ alkylene, C₂₋₆ alkenylene, C₂₋₆ alkynylene, C₃₋₁₀ cycloalkylene, heterocyclylene, arylene, or heteroaryl of A is independently optionally substituted with one to five Z^{A};
Ring B is a 5- or 6-membered heteroaryl optionally substituted with one to three R^{B};
each R^{B} is independently selected from halo, hydroxy, —NH₂, cyano, C₁₋₃ alkyl, C₁₋₃ haloalkyl, C₁₋₃ alkoxy, and C₁₋₃ haloalkoxy; wherein each C₁₋₃ alkyl of R^{B} is independently optionally substituted with —NH₂, —NHC₁₋₃ alkyl, —N(C₁₋₃ alkyl)₂, hydroxy, or C₁₋₃ alkoxy;
L¹ is C₁₋₃ alkylene, C₂₋₃ alkenylene, C₂₋₃ alkynylene, C₁₋₃ heteroalkylene, C₃₋₆ cycloalkylene, or 4-6 membered heterocyclylene; wherein the C₁₋₃ alkylene, C₂₋₃ alkenylene, C₂₋₃ alkynylene, C₁₋₃ heteroalkylene, C₃₋₆ cycloalkylene, or 4-6 membered heterocyclylene of L¹ is independently optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
L² is a bond, —O—, —S—, —NR^{2a}—, —C(O)—, —C(O)O—, —OC(O)—, —OC(O)O—, —C(O)NR^{2a}—, —NR^{2a}C(O)—, —OC(O)NR^{2a}—, —NR^{2a}C(O)O—, —NR^{2a}C(O)NR^{2b}—, —S(O)—, —S(O)₂—, —S(O)NR^{2a}—, —S(O)₂NR^{2a}—, —NR^{2a}S(O)—, —NR^{2a}S(O)₂—, —NR^{2a}S(O)NR^{2b}—, —NR^{2a}S(O)₂NR^{2b}—, C₁₋₆ alkylene, C₂₋₆ alkenylene, C₂₋₆ alkynylene, C₁₋₆ heteroalkylene, C₃₋₆ cycloalkylene, 4-6 membered heterocyclylene, or 5 membered heteroarylene; wherein the C₁₋₃ alkylene, C₂₋₃ alkenylene, C₂₋₃ alkynylene, C₁₋₃ heteroalkylene, C₃₋₆ cycloalkylene, 4-6 membered heterocyclylene, or 5 membered heteroarylene of L² is independently optionally substituted with one to five substituents independently selected from halo, oxo, hydroxy, cyano, —NH₂, —NHC₁₋₃ alkyl, —N(C₁₋₃ alkyl)₂, C₁₋₃ alkyl, C₁₋₃ haloalkyl, C₁₋₃ alkoxy, and C₁₋₃ haloalkoxy;
R¹ is cyano, —C(O)NR^{1a}R^{1b}, —C(S)NR^{1a}R^{1b}, —S(O)₂R², —S(O)(NR⁶)R², or —P(O)R⁷R²;
R² is —NR^{1a}R^{1b}, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl of R² is optionally substituted with one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, or C₁₋₃ haloalkoxy;
or R² and R⁶, or R² and R⁷, together with the atom to which they are attached, form a heterocyclyl optionally substituted with one to five Z²;
each R^{1a} and R^{1b} is independently hydrogen, C₁₋₃ alkyl, C₂₋₃ alkenyl, C₂₋₃ alkynyl, C₁₋₃ haloalkyl, C₃₋₆ cycloalkyl, or 4-6 membered heterocyclyl; wherein each C₁₋₃ alkyl, C₂₋₃ alkenyl, C₂₋₃ alkynyl, C₁₋₃ haloalkyl, C₃₋₆ cycloalkyl, or 4-6 membered heterocyclyl of R^{1a} and R^{1b} is independently optionally substituted with one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, or C₁₋₃ haloalkoxy;
or R^{1a} and R^{1b} are taken together with the atoms to which they are attached to form 4-6 membered heterocyclyl independently optionally substituted by one to five substituents independently selected from halo, hydroxy, cyano, C₁₋₃ alkyl, C₂₋₃ alkenyl, C₂₋₃ alkynyl, C₁₋₃ haloalkyl, C₁₋₃ alkoxy, or C₁₋₃ haloalkoxy;
each R^{2a} and R^{2b} is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{2a} and R^{2b} is independently optionally substituted with one to five Z^{2a};
or R^{2a} and R^{2b} are taken together with the atoms to which they are attached to form heterocyclyl independently optionally substituted by one to five Z^{2a};
R³ is hydrogen, —NR^{3b}R^{3c}, C₁₋₆ alkyl, C₂₋₆ alkenyl, C₂₋₆ alkynyl, C₁₋₆ haloalkyl, C₁₋₆ alkoxy, C₁₋₆ haloalkoxy, C₁₋₆ heteroalkyl, C₁₋₆ haloalkyl, C₁₋₆ haloalkoxy, C₃₋₁₀ cycloalkyl, heterocyclyl, aryl, or heteroaryl; wherein the C₁₋₆ alkyl, C₂₋₆ alkenyl, C₂₋₆ alkynyl, C₁₋₆ haloalkyl, C₁₋₆ alkoxy, C₁₋₆ heteroalkyl, C₁₋₆ haloalkyl, C₁₋₆ haloalkoxy, C₃₋₁₀ cycloalkyl, heterocyclyl, aryl, or heteroaryl of R³ is independently optionally substituted with one to five Z³;
R^{3b} and R^{3c} are each independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R^{3b} and R^{3c} is independently optionally substituted with one to five substituents independently selected from halo, hydroxy, cyano, C₁₋₃ alkyl, C₁₋₃ haloalkyl, C₁₋₃ alkoxy, and C₁₋₃ haloalkoxy;
or R^{3b} and R^{3c} are taken together with the nitrogen atom to which they are attached to form a heterocyclyl optionally substituted with one to five Z^{3b};
R⁴ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl of R⁴ is independently optionally substituted with one to five Z⁴;
R⁵ is hydrogen, halo, hydroxy, amino, cyano, C₁₋₆ alkyl, C₁₋₆ alkoxy, C₁₋₆ haloalkyl, C₃₋₁₀ cycloalkyl, heterocyclyl, aryl, or heteroaryl; wherein the C₁₋₆ alkyl, C₁₋₆ alkoxy, C₁₋₆ haloalkyl, C₃₋₁₀ cycloalkyl, heterocyclyl, aryl, or heteroaryl of R⁵ is independently optionally substituted with one to five Z⁵;
R⁶ is hydrogen, C₁₋₃ alkyl, C₁₋₃ haloalkyl, C₃₋₆ cycloalkyl, or 4 to 6-membered heterocyclyl; wherein the C₁₋₃ alkyl, C₁₋₃ haloalkyl, C₃₋₆ cycloalkyl, or 4 to 6-membered heterocyclyl is optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy;
R⁷ is hydroxy, C₁₋₃ alkoxy, C₁₋₃ haloalkoxy, C₁₋₃ alkyl, or C₁₋₃ haloalkyl;
each Z^{A}, Z², Z^{2a}, Z³, Z^{3b}, Z⁴, and Z⁵; is independently halo, cyano, nitro, oxo, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-H, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{A}, Z², Z^{2a}, Z³, Z^{3b}, Z⁴, and Z⁵ are each independently optionally substituted with one to five Z^{1a};
each L is independently —O—, —S—, —NR²⁰—, —C(O)—, —C(O)O—, —OC(O)—, —OC(O)O—, —C(O)NR²⁰—, —NR²⁰C(O)—, —OC(O)NR²⁰—, —NR²⁰C(O)O—, —NR²⁰C(O)NR²¹—, —S(O)—, —S(O)₂—, —S(O)NR²⁰—, —S(O)₂NR²⁰—, —NR²⁰S(O)—, —NR²⁰S(O)₂—, —NR²⁰S(O)NR²¹—, or —NR²⁰S(O)₂NR²¹—;
each R²⁰ and R²¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R²⁰ and R²¹ is independently optionally substituted with one to five Z^{1a}; or an R²⁰ and R²¹ are taken together with the atoms to which they are attached to form heterocyclyl independently optionally substituted by one to five Z^{1a}; and
each Z^{1a} is independently halo, hydroxy, cyano, nitro, oxo, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of Z^{1a} is independently optionally substituted with one to five substituents independently selected from C₁₋₆ alkyl, oxo, halo, hydroxy, and cyano;
provided that when A is C₁₋₆ alkylene; then R⁵ is C₃₋₁₀ cycloalkyl, heterocyclyl, aryl, or heteroaryl; wherein the C₃₋₁₀ cycloalkyl, heterocyclyl, aryl, or heteroaryl is independently optionally substituted.

2. The compound of claim 1, wherein R¹ is —C(O)NR^{1a}R^{1b}, —S(O)₂R², —S(O)(NR⁶)R², or —P(O)R⁷R².

3. The compound of claim 1, wherein R¹ is —C(O)NH₂, —S(O)₂NH₂, —S(O)₂CH₃, 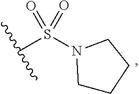 [SMILES: *S(=O)(=O)N1CCCC1]
—S(O)(NH)CH₃, or —P(O)(CH₃)CH₃.

4. The compound of claim 1, wherein the compound is represented by Formula IA: 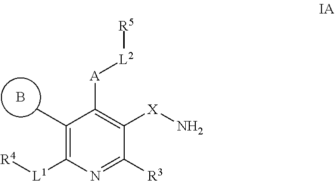
or a pharmaceutically acceptable salt, stereoisomer, mixture of stereoisomers, or solvate thereof, wherein X is —C(O)— or —S(O)₂—.

5. The compound of claim 1, wherein A is C₁₋₆ alkylene, C₃₋₁₀ cycloalkylene, heterocyclylene, arylene, or heteroarylene; wherein the C₁₋₆ alkylene, C₃₋₁₀ cycloalkylene, heterocyclylene, arylene, or heteroarylene of A is independently optionally substituted with one to five Z^{A}.

6. The compound of claim 1, wherein A is C₃₋₁₀ cycloalkylene, heterocyclylene, arylene, or heteroarylene; wherein the C₃₋₁₀ cycloalkylene, heterocyclylene, arylene, or heteroarylene is independently optionally substituted with one to five Z^{A}.

7. The compound of claim 1, wherein A is methylene, ethylene, n-propylene, 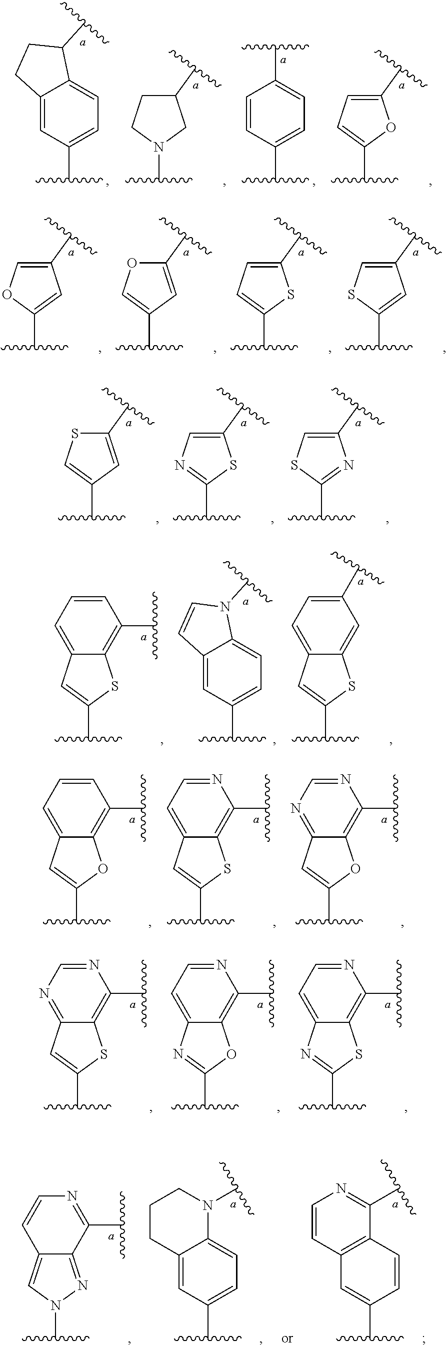
wherein bond a is bonded to L².

8. The compound of claim 1, wherein the compound is represented by Formula IB: 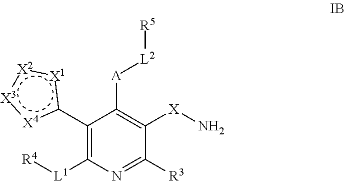
or a pharmaceutically acceptable salt, stereoisomer, mixture of stereoisomers, or solvate thereof, wherein:
X is —C(O)— or —S(O)₂—; and
each of X¹, X², X³, and X⁴ are independently selected from O, S, N, NR^{B}, and CR^{B}; provided that at least one of X¹, X², X³, and X⁴ the ring encompassing X¹, X², X³, and X⁴ is aromatic.

9. The compound of claim 1, wherein A is arylene or heteroarylene; wherein the arylene or heteroarylene is independently optionally substituted with one to five Z^{A}.

10. The compound of claim 1, wherein Ring B is pyridyl, pyrazolyl, isoxazolyl, oxadiazolyl, or thiadiazolyl; wherein the pyridyl, pyrazolyl, isoxazolyl, oxadiazolyl, or thiadiazolyl optionally substituted with one or two R^{B}.

11. The compound of claim 1, wherein Ring B or the moiety 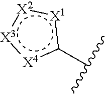
is: 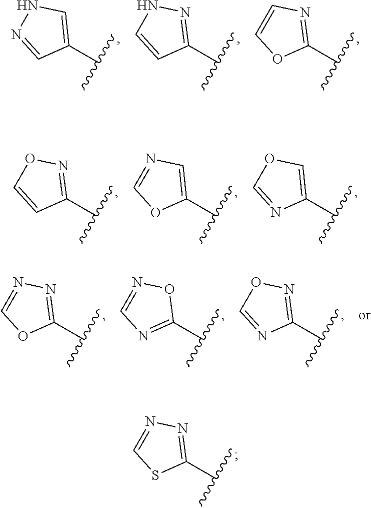
wherein each is optionally substituted with one or two R^{B}.

12. The compound of claim 1, wherein each R^{B} is independently C₁₋₃ alkyl.

13. The compound of claim 1, wherein L¹ is C₃₋₆ cycloalkylene, C₁₋₃ alkylene, or C₁₋₃ heteroalkylene; wherein each C₃₋₆ cycloalkylene, C₁₋₃ alkylene or C₁₋₃ heteroalkylene is independently optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy.

14. The compound of claim 1, wherein R⁴ is C₁₋₆ alkyl, C₃₋₁₀ cycloalkyl, aryl, heterocyclyl, or heteroaryl; wherein the C₁₋₆ alkyl, C₃₋₁₀ cycloalkyl, aryl, heterocyclyl, or heteroaryl is independently optionally substituted with one to five Z⁴.

15. The compound of claim 1, wherein R⁴ is aryl, heterocyclyl, or heteroaryl; wherein the aryl, heterocyclyl, or heteroaryl is optionally substituted with one to five Z⁴.

16. The compound of claim 1, wherein R⁴ is C₁₋₃ alkyl, C₃₋₉ cycloalkyl, 4-9 membered heterocyclyl, or phenyl; wherein the C₁₋₃ alkyl, C₃₋₆ cycloalkyl, or phenyl is independently optionally substituted with one to three halo.

17. The compound of claim 1, wherein R³ is C₁₋₆ alkyl or C₁₋₆ haloalkyl.

18. The compound of claim 1, wherein R³ is hydrogen, —NR^{3b}R^{3c}, C₁₋₆ alkyl, or C₁₋₆ alkoxy.

19. The compound of claim 1, wherein L² is a bond, —NR^{2a}—, —C(O)NR^{2a}—, —NR^{2a}C(O)—, C₁₋₆ alkylene, C₂₋₆ alkenylene, C₂₋₆ alkynylene, C₁₋₆ heteroalkylene, 4-6 membered heterocyclylene, or 5 membered heteroarylene; wherein the C₁₋₆ alkylene, C₂₋₆ alkenylene, C₂₋₆ alkynylene, C₁₋₆ heteroalkylene, 4-6 membered heterocyclylene, or 5 membered heteroarylene is independently optionally substituted with one to five substituents independently selected from halo, oxo, hydroxy, cyano, C₁₋₃ alkyl, C₁₋₃ haloalkyl, C₁₋₃ alkoxy, and C₁₋₃ haloalkoxy.

20. The compound of claim 1, wherein R⁵ is hydrogen, halo, amino, cyano, C₁₋₆ alkyl, C₁₋₆ alkoxy, C₃₋₁₀ cycloalkyl, heterocyclyl, aryl, or heteroaryl; wherein the C₁₋₆ alkyl, C₁₋₆ alkoxy, C₃₋₁₀ cycloalkyl, heterocyclyl, aryl, or heteroaryl is independently optionally substituted with one to five Z⁵.

21. The compound of claim 1, wherein R⁵ is aryl or heteroaryl; wherein the aryl or heteroaryl is optionally substituted with one to five Z⁵.

22. The compound of claim 1, wherein R⁵ is hydrogen, halo, amino, cyano, C₁₋₆ alkyl, C₁₋₆ alkoxy, C₃₋₁₀ cycloalkyl, heterocyclyl, or aryl; wherein the C₁₋₆ alkyl, C₁₋₆ alkoxy, C₃₋₁₀ cycloalkyl, heterocyclyl, or aryl is independently optionally substituted with one to five Z⁵.

23. A compound, or a pharmaceutically acceptable salt, stereoisomer, mixture of stereoisomers, or solvate thereof, as shown in Table 1 or Table 2.

24. A pharmaceutical composition comprising a compound of claim 1, or a pharmaceutically acceptable salt, stereoisomer, mixture of stereoisomers, or prodrug thereof, and a pharmaceutically acceptable excipient.

25. A method for treating a calcitonin receptor and/or an amylin receptor associated disease or disorder in a subject in need thereof, the method comprising administering to a subject in need thereof a therapeutically effective amount of a compound of claim 1, or a pharmaceutically acceptable salt, stereoisomer, mixture of stereoisomers, or solvate thereof.

26. The method of claim 25, wherein the calcitonin receptor and/or amylin receptor associated disease or disorder is a bone disorder, a metabolic disorder, pain, a neurodegenerative disease or disorder, a cardiovascular disease, or other disease or disorder.

27. The method of claim 25, wherein the calcitonin receptor and/or amylin receptor associated disease or disorder is osteoporosis, Paget's disease, hypercalcemia, Sudeck's atrophy, polystatic fibrous displasia, intersemocostoclavicular ossification, osteogenesis imperfecta, osteopenia, periodontal disease or defect, osteolytic bone disease, metastatic bone disorder, bone loss resulting from a malignancy, autoimmune arthritides, a breakage or fracture, or immobility or disuse, osteopathic pain, phantom limb pain, general pain, hyperalgesia, pain associated with diabetic neuropathy, non-alcoholic fatty liver disease (NAFLD), non-alcoholic steatohepatitis (NASH), Alzheimer's disease, insulin dependent diabetes, non-insulin dependent diabetes, impaired glucose tolerance, obesity, syndrome X, a diabetic complication, primary or secondary hyperthyroidism, endocrine disorder, conditions associated with inhibiting gastric secretion, gastrointestinal disorders, renal osteodystrophy, or male infertility.

28. The method of claim 25, further comprising administering an additional therapy or therapeutic agent to the patient.

29. The method of claim 28, wherein the additional therapy or therapeutic agent is selected from the group consisting of an antidiabetic agent, an anti-obesity agent, a weight loss agent, a GLP-1 receptor agonist, an anti-emetic agent, an agent to treat non-alcoholic steatohepatitis (NASH), gastric electrical stimulation, dietary monitoring, physical activity, or a combination thereof.
